(19) United States
(12) Patent Application Publication
(10) Pub. No.: US 2025/0011318 A1
(43) Pub. Date: Jan. 9, 2025

(54) CBL-B INHIBITORS AND METHODS OF USE THEREOF

(71) Applicant: ARCUS BIOSCIENCES, INC., Hayward, CA (US)

(72) Inventors: Srikanth Kumar Gangam, Fremont, CA (US); Clayton Hardman, San Francisco, CA (US); Kenneth Victor Lawson, San Francisco, CA (US); Manmohan Reddy Leleti, Dublin, CA (US); Dongdong Liu, Fremont, CA (US); Artur Karenovich Mailyan, Livermore, CA (US); Masa Podunavac, Hayward, CA (US); Shiwei Qu, Alameda, CA (US); Zhang Wang, Foster City, CA (US); Xianglin Yin, Hayward, CA (US); Kai Yu, Hayward, CA (US); Tzu-Yu Yu, Hayward, CA (US)

(73) Assignee: ARCUS BIOSCIENCES, INC., Hayward, CA (US)

(21) Appl. No.: 18/673,794

(22) Filed: May 24, 2024

Related U.S. Application Data
(60) Provisional application No. 63/469,022, filed on May 25, 2023.
(60) Provisional application No. 63/623,146, filed on Jan. 19, 2024.

(51) Int. Cl. C07D 471/04; A61K 31/444; A61K 31/4545; A61K 31/4995; A61K 31/501; A61K 31/519; A61K 31/5377; A61K 31/5386; A61K 31/553; A61K 45/06; C07D 487/04; C07D 519/00

(52) U.S. Cl. CPC C07D 471/04; A61K 31/444; A61K 31/4545; A61K 31/4995; A61K 31/501; A61K 31/519; A61K 31/5377; A61K 31/5386; A61K 31/553; A61K 45/06; C07D 487/04; C07D 519/00

(57) ABSTRACT

Disclosed herein are compounds having a structure according to Formula I, and compositions containing those compounds. Methods of preparing the compounds, and methods of using the compounds for the treatment of diseases, disorders, or conditions are also described.

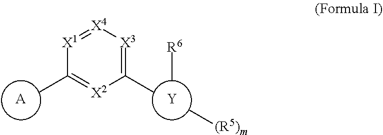

### CROSS-REFERENCE TO RELATED APPLICATIONS

This application claims the benefit of priority to U.S. Provisional Patent Application No. 63/469,022, filed on May 25, 2023, and U.S. Provisional Patent Application No. 63/623,146, filed on Jan. 19, 2024, the entire contents of each of which is incorporated by reference herein.

### BACKGROUND

The following discussion is provided to aid the reader in understanding the disclosure and is not admitted to describe or constitute prior art thereto.

Crucial to the proper regulation of immune responses is a balance between immune activating and inhibitory signals. One cellular mechanism that regulates diverse aspects of the immune system is ubiquitination. Ubiquitination involves covalent conjugation of monoubiquitin or polyubiquitin chains onto amino acid residues of target proteins. Protein ubiquitination can alter the activity and/or stability of a molecule, and in some instances can also alter localization of the molecule into different cellular compartments. The ubiquitination process is catalyzed by sequential actions of ubiquitin-activating (E1), ubiquitin-conjugating (E2) and ubiquitin-ligating (E3) enzymes. The process of protein ubiquitination is counteracted by deubiquitinases (DUBs), a large family of proteases that cleaves ubiquitin chains. Mammalian cells express more than 600 E3 ligases and about 100 DUBs, which display substrate specificities and regulate specific cellular functions.

An increasing number of E3 ligases and DUBs have been identified as important regulators of immune responses. For example, small-molecule inhibitors that are antagonists of the IAP family of E3 ligases, including cIAP1, cIAP2, and X-linked IAP (XIAP), have been developed as small-molecule mimetics of the endogenous IAP inhibitor Smac. Small molecule inhibitors have also been developed against MDM2, an E3 ligase that promotes tumor growth and progression by mediating ubiquitin-dependent degradation of the tumor suppressor p53 and p53-independent functions. Casitas B-lineage lymphoma (Cbl) proteins, a family of E3 ubiquitin ligases, have been previously identified as potential targets; and so has VHL E3 complex, which mediates ubiquitin-dependent degradation of HIF1α and controls metabolic activities and effector function of T cells. Small molecule inhibitors for several DUBs have also been developed, and some of them have been shown to inhibit tumor growth in animal models. The mammalian Cbl family contains three homologs—c-Cbl, Cbl-b, and Cbl-3. Cbl-b and c-Cbl share some structural similarities but may have distinct physiological functions.

While these potential targets provide exciting opportunities for the immunotherapy field, there remains a need in the art for effective inhibitors.

### SUMMARY

In one aspect, this disclosure is directed to a compound having a structure according to Formula I:

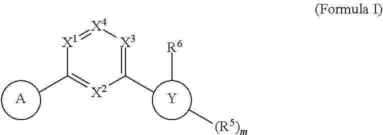

or a pharmaceutically acceptable salt thereof, wherein:
A has a formula selected from the group consisting of:

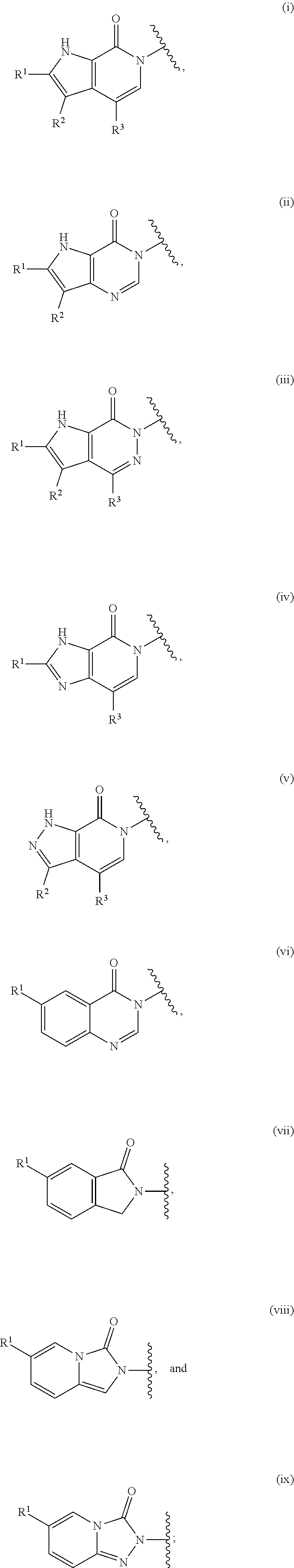

wherein:
R¹ is selected from the group consisting of —H, —C₁-C₆ alkyl, —C₁-C₆ haloalkyl, —C₁-C₆ hydroxyalkyl, -(Q¹)-O—(C₁-C₃ alkyl), —C(O)NH₂, —C(O)—(C₁-C₆-alkyl), -(Q¹)-NR^{1a}R^{1b},
-(Q¹)-(C₃-C₇ cycloalkyl), -(Q¹)-(5- to 6-membered heteroaryl), and -(Q¹)-(4- to 8-membered heterocycloalkyl); wherein said 5- to 6-membered heteroaryl has 1-3 ring heteroatoms independently selected from N, O, and S; said 4- to 8-membered heterocycloalkyl has 1-3 ring heteroatom or heteroatom groups independently selected from N, O, S, and S(O)₂; said C₁-C₃ alkyl is unsubstituted or substituted with —C₁-C₃ alkoxy; and said C₃-C₇ cycloalkyl, 4- to 8-membered heterocycloalkyl, and 5- to 6-membered heteroaryl are unsubstituted or substituted with 1-2 substituents independently selected from halo, —OH, —C₁-C₃ alkyl, —C₁-C₃ alkoxy, and —C(O)(C₁-C₃ alkyl);
Q¹ is absent, unsubstituted —(C₁-C₃ alkylene)-, or —(C₁-C₃ alkylene)-substituted with 1-3 R^{q};
each R^{q} is independently halo, —OH, or —NH₂;
R^{1a} and R^{1b} are independently selected from the group consisting of —H, —C₁-C₆ alkyl, —C₁-C₆ haloalkyl, phenyl, —(C₁-C₃ alkylene)-O—(C₁-C₃ alkyl), —C₃-C₆ cycloalkyl, —(C₁-C₃ alkylene)-(C₃-C₆ cycloalkyl), —S(O)₂(C₁-C₆ alkyl), 5- to 6-membered heteroaryl having 1-3 ring heteroatoms independently selected from N, O, and S, and 4- to 8-membered heterocycloalkyl having 1-3 ring heteroatom or heteroatom groups independently selected from N, O, S, and S(O)₂; wherein said phenyl, —(C₁-C₃ alkylene)-O—(C₁-C₃ alkyl), —C₃-C₆ cycloalkyl, —(C₁-C₃ alkylene)-(C₃-C₆ cycloalkyl), 5- to 6-membered heteroaryl, and 4- to 8-membered heterocycloalkyl are unsubstituted or substituted with 1-3 R^{1c};
each R^{1c}, when present, is independently halo, —OH, —C₁-C₃ alkyl, —C₁-C₃ hydroxyalkyl, or —C₁-C₃ haloalkyl;
R², when present, is —H, halo, —CN, —C₁-C₃ alkyl, —C₁-C₃ haloalkyl, —C₃-C₄ cycloalkyl,
—S(O)₂(C₁-C₃ alkyl), —C(O)—NR^{2a}R^{2b}, or 5- to 6-membered heteroaryl having 1-3 ring heteroatoms independently selected from N, O, and S; or
R¹ and R² taken together with the atoms to which they are attached form —C₃-C₆ cycloalkyl;
R^{2a} and R^{2b} are independently —H, or —C₁-C₃ alkyl;
R³, when present, is —H, —CN, halo, —C₁-C₆ alkyl, —C₁-C₆ haloalkyl, —C₁-C₆ hydroxyalkyl, —C₂-C₃ alkenyl, —C₃-C₄ cycloalkyl, —S(O)₂(C₁-C₆ alkyl), —C(O)OH, or 5- to 6-membered heteroaryl having 1 to 4 ring heteroatoms independently selected from N, S, and O; and the 5- to 6-membered heteroaryl is unsubstituted or substituted with 1-3 substituents independently selected from —C₁-C₃ alkyl;
X¹, X² and X³ are each independently N or CH;
X⁴ is N or CR⁴;
R⁴, when present, is —H, halo, —CN, —OH, —C₁-C₆ alkyl, —C₁-C₆ haloalkyl, —C₁-C₆ alkoxy, —NR^{4a}R^{4b} or —C₃-C₈ cycloalkyl;
R^{4a} and R^{4b} are independently —H, —C₁-C₃ alkyl, or —(C₁-C₃ alkylene)-NR^{4c}R^{4d};
R^{4c} and R^{4d} are independently —H, or —C₁-C₃ alkyl;
Y is phenyl, or 5- to 6-membered heteroaryl having 1-3 ring heteroatoms independently selected from N, O, and S;
m is 0, 1, 2, or 3;
each R⁵ when present, is independently halo, —CN, —C₁-C₆ alkyl, —C₁-C₆ haloalkyl, or —C₁-C₆ alkoxy;
when ring A has a structure of formula (i), (ii), (iii), (iv), (v), or (vi), R⁶ is a 5- to 6-membered heteroaryl having 1 to 4 ring heteroatoms independently selected from N, O, and S, wherein R⁶ is unsubstituted or substituted with 1-3 R^{6a}; or
when ring A has a structure of formula (vii), (viii), or (ix), R⁶ is a 6-membered heteroaryl having 1 to 4 ring heteroatoms independently selected from N, O, and S, wherein R⁶ is unsubstituted or substituted with 1-3 R^{6a}; and
each R^{6a} is independently selected from the group consisting of —CN, —C₁-C₃ alkyl, —C₁-C₃ haloalkyl, and —C₁-C₃ hydroxyalkyl.

In another aspect, this disclosure is directed to methods of inhibiting Cbl-b in a subject comprising administering to the subject an effective amount of a compound described herein.

In another aspect, this disclosure is directed to methods of increasing immune cell activity in a subject comprising administering to the subject an effective amount of a compound described herein.

In yet another aspect, this disclosure provides methods for treating a disease, disorder, or condition mediated at least in part by Cbl-b in a subject, comprising administering to the subject a therapeutically effective amount of a compound described herein. Diseases, disorders, and conditions mediated by Cbl-b include cancer and cancer-related disorders.

Certain aspects of the present disclosure further comprise the administration of one or more additional therapeutic agents as set forth herein below.

### DETAILED DESCRIPTION OF THE DISCLOSURE

Before the present disclosure is further described, it is to be understood that the disclosure is not limited to the particular embodiments set forth herein, and it is also to be understood that the terminology used herein is for the purpose of describing particular embodiments only, and is not intended to be limiting.

### Definitions

Unless otherwise defined, all terms of art, notations and other scientific terms or terminology used herein are intended to have the meanings commonly understood by those of skill in the art to which this disclosure pertains.

The term “about” as used herein has its original meaning of approximately and is to provide literal support for the exact number that it precedes, as well as a number that is near to or approximately the number that the term precedes. In general, the term “about” refers to the usual error range for the respective value readily known to the skilled person in this technical field. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The term “alkyl”, by itself or as part of another substituent, means, unless otherwise stated, a saturated monovalent hydrocarbon radical, having, in some embodiments, one to eight (e.g., C₁-C₈ alkyl), or one to six (e.g., C₁-C₆ alkyl), or one to three (e.g., C₁-C₃ alkyl) carbon atoms, respectively. The term “alkyl” encompasses straight and branched-chain hydrocarbon groups. Examples of alkyl groups include, but are not limited to, methyl (Me), ethyl (Et), n-propyl, isopropyl, n-butyl, t-butyl, isobutyl, sec-butyl, isopentyl, tert-pentyl, n-pentyl, isohexyl, n-hexyl, n-heptyl, 4-isopropylheptane, n-octyl, and the like. In some embodiments, the alkyl groups are C₁-C₄ alkyl groups (e.g., methyl, ethyl, isopropyl, or t-butyl). In some embodiments, the alkyl groups are C₁-C₃ alkyl groups (e.g., methyl, ethyl, n-propyl, or isopropyl).

The term “alkenyl”, as used herein, refers to a straight or branched monovalent hydrocarbon radical having, in some embodiments, two to eight carbon atoms (e.g., C₂-C₈ alkenyl), or two to six carbon atoms (e.g., C₂-C₆ alkenyl), or two to three carbon atoms (e.g., C₂-C₃ alkenyl), and having at least one carbon-carbon double bond. Examples of alkenyl groups include, but are not limited to, ethenyl, propenyl, isobutenyl, butadienyl and the like.

The term “alkylene” refers to a straight or branched, saturated, hydrocarbon radical having, in some embodiments, one to six (e.g., C₁-C₆ alkylene), one to four (e.g., C₁-C₄ alkylene), one to three (e.g., C₁-C₃ alkylene), or one to two (e.g., C₁-C₂ alkylene) carbon atoms, and linking at least two other groups, i.e., a divalent hydrocarbon radical. The two moieties linked to the alkylene can be attached to the same carbon atom (i.e., geminal), or different carbon atoms of the alkylene group. For instance, a straight chain alkylene can be the bivalent radical of —(CH₂)ₙ—, where n is 1, 2, 3, 4, 5 or 6 (i.e., a C₁-C₆ alkylene). Representative alkylene groups include, but are not limited to, methylene, ethylene, propylene, isopropylene, butylene, isobutylene, secbutylene, pentylene, hexylene and the like. In some embodiments, the alkylene groups are C₁-C₂ alkylene groups (e.g., methylene, or ethylene). In some embodiments, the alkylene groups are C₁-C₃ alkylene groups (e.g., methylene, ethylene, or propylene).

As used herein, the term “alkoxy” refers to an alkyl group, as defined herein, that is attached to the remainder of the molecule via an oxygen atom (e.g., —O—C₁-C₁₂ alkyl, —O—C₁-C₅ alkyl, —O—C₁-C₆ alkyl, or —O—C₁-C₃ alkyl). Non-limiting examples of alkoxy groups include methoxy, ethoxy, n-propoxy, iso-propoxy, n-butoxy, sec-butoxy, n-pentoxy, n-hexoxy, and the like. In some embodiments, the alkoxy groups are C₁-C₃ alkoxy groups (e.g., methoxy, ethoxy, n-propoxy, or iso-propoxy).

The term “cycloalkyl” refers to a monocyclic, bicyclic or polycyclic hydrocarbon ring system having, in some embodiments, 3 to 14 carbon atoms (e.g., C₃-C₁₄ cycloalkyl), or 3 to 10 carbon atoms (e.g., C₃-C₁₀ cycloalkyl), or 3 to 8 carbon atoms (e.g., C₃-C₈ cycloalkyl), or 3 to 6 carbon atoms (e.g., C₃-C₆ cycloalkyl) or 3 to 4 carbon atoms (e.g., C₃-C₄ cycloalkyl). Cycloalkyl groups can be saturated or characterized by one or more points of unsaturation (i.e., carbon-carbon double and/or triple bonds), provided that the points of unsaturation do not result in an aromatic system. Examples of monocyclic cycloalkyl groups include, but are not limited to, cyclopropyl, cyclobutyl, cyclopentyl, cyclohexyl, cyclohexenyl, cycloheptyl, cycloheptenyl, cycloheptadienyl, cyclooctyl, cyclooctenyl, cyclooctadienyl and the like. The rings of bicyclic and polycyclic cycloalkyl groups can be fused, bridged, or spirocyclic. Non-limiting examples of bicyclic, spirocyclic and polycyclic cycloalkyl groups include bicyclo[2.2.1]heptane, bicyclo[2.2.2]octane, adamantyl, indanyl, spiro[5.5]undecane, spiro[2.2]pentane, spiro[2.2]pentadiene, spiro[2.3]hexane, spiro[2.5]octane, spiro[2.2]pentadiene, and the like. In some embodiments, the cycloalkyl groups of the present disclosure are monocyclic C₃-C₆ cycloalkyl moieties (e.g., cyclopropyl, cyclobutyl, cyclopentyl, or cyclohexyl). In some embodiments, the cycloalkyl groups of the present disclosure are monocyclic C₃-C₄ cycloalkyl moieties (e.g., cyclopropyl, or cyclobutyl).

The term “heterocycloalkyl” refers to a non-aromatic monocyclic, bicyclic or polycyclic cycloalkyl ring having, in some embodiments, 3 to 14 members (e.g., 3- to 14-membered heterocycle), or 3 to 10 members (e.g., 3- to 10-membered heterocycle), or 3 to 8 members (e.g., 3- to 8-membered heterocycle), or 3 to 6 members (e.g., 3- to 6-membered heterocycle), or 5 to 6 members (e.g., 5- to 6-membered heterocycle), and having from one to five, one to four, one to three, one to two or one heteroatom or heteroatom groups independently selected from nitrogen (N), oxygen (O), sulfur (S), sulfoxide (S(O)), and sulfone (S(O)₂). Heterocycloalkyl groups are saturated or characterized by one or more points of unsaturation (e.g., one or more carbon-carbon double bonds, carbon-carbon triple bonds, carbon-nitrogen double bonds, and/or nitrogen-nitrogen double bonds), provided that the points of unsaturation do not result in an aromatic system. The rings of bicyclic and polycyclic heterocycloalkyl groups can be fused, bridged, or spirocyclic. Non-limiting examples of heterocycloalkyl groups include aziridine, oxirane, thiirane, pyrrolidine, imidazolidine, pyrazolidine, dioxolane, phthalimide, piperidine, 1,4-dioxane, morpholine, thiomorpholine, thiomorpholine-S-oxide, thiomorpholine-S,S-oxide, piperazine, 3,4,5,6-tetrahydropyridazine, tetrahydropyran, pyran, decahydroisoquinoline, 3-pyrroline, thiopyran, tetrahydrofuran, tetrahydrothiophene, tetrahydro-1,1-dioxido-2H-thiopyran, quinuclidine, 1,4-oxazepane, 2-azabicyclo[4.1.0]heptane, 2-oxa-5-azabicyclo[2.2.1]heptane, 2-azabicyclo[2.2.1]heptane, 2,5-diazabicyclo[2.2.1]heptane, 6-oxa-3-azabicyclo[3.1.1]heptane, 3-oxa-6-azabicyclo[3.1.1]heptane, 2,5-diazabicyclo[2.2.1]heptane, 2-thia-6-azaspiro[3.3]heptane 2,2-dioxide, 2,6-diazaspiro[3.3]heptane, 2-azaspiro[3.3]heptane, 1-oxaspiro[3.3]heptane, 5-azaspiro[2.4]heptane, 6-azaspiro[3.4]octane, 6-azaspiro[2.5]octane, 4-oxa-7-azaspiro[2.5]octane, 3-oxa-8-azabicyclo[3.2.1]octane, and the like. A heterocycloalkyl group can be attached to the remainder of the molecule through a ring carbon atom, or a ring heteroatom, when chemically permissible. In some embodiments, the heterocycloalkyl groups of the present disclosure are monocyclic 4- to 8-membered heterocycloalkyl moieties having one or two heteroatom or heteroatom groups independently selected from N, O, S, and S(O)₂ (e.g., azetidine, oxetane, piperidine, piperazine, morpholine, pyrrolidine, imidazolidine, pyrazolidine, tetrahydrofuran, tetrahydropyran, 1,4-oxazepane, 2-azabicyclo[2.2.1]heptane, 2,5-diazabicyclo[2.2.1]heptane, 6-oxa-3-azabicyclo[3.1.1]heptane, 3-oxa-6-azabicyclo[3.1.1]heptane, 2-thia-6-azaspiro[3.3]heptane 2,2-dioxide, and the like).

The term “aryl” refers to an aromatic ring system containing one ring, or two or three rings fused together, and having, in some embodiments, six to fourteen (i.e., C₆-C₁₄ aryl), or six to ten (i.e., C₆-C₁₀ aryl), or six (i.e., C₆ aryl) carbon atoms. Non-limiting examples of aryl groups include phenyl, naphthyl and anthracenyl. In some embodiments, aryl groups are phenyl.

The term “heteroaryl” refers to monocyclic or fused bicyclic aromatic groups (or rings) having, in some embodiments, from 5 to 14 (i.e., 5- to 14-membered heteroaryl), or from 5 to 10 (i.e., 5- to 10-membered heteroaryl), or from 5 to 6 (i.e., 5- to 6-membered heteroaryl) members (i.e., ring vertices), and containing from one to five, one to four, one to three, one to two or one heteroatom independently selected from nitrogen (N), oxygen (O), and sulfur (S). A heteroaryl group can be attached to the remainder of the molecule through a carbon atom or a heteroatom of the heteroaryl group, when chemically permissible. Non-limiting examples of heteroaryl groups include pyridyl, pyridazinyl, pyrazinyl, pyrimidinyl, triazinyl, purinyl, thienopyridinyl, thienopyrimidinyl, pyrazolopyrimidinyl, imidazopyridines, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like. In some embodiments, the heteroaryl groups of the present disclosure are monocyclic 5- to 6-membered heteroaryl moieties having 1-3 heteroatoms independently selected from N, O, and S (e.g., pyridinyl, pyrimidinyl, pyridazinyl, triazolyl, imidazolyl, pyrazolyl, oxazolyl, oxadiazolyl, or thiazolyl).

As used herein, a wavy line, “ [symbol] ”, that intersects a single, double or triple bond in any chemical structure depicted herein, represents that the point of attachment of the single, double, or triple bond to the remainder of the molecule is through either one of the atoms that make up the single, double or triple bond. Additionally, a bond extending from a substituent to the center of a ring (e.g., a phenyl ring) is meant to indicate attachment of that substituent to the ring at any of the available ring vertices, i.e., such that attachment of the substituent to the ring results in a chemically stable arrangement.

The term “halogen,” by itself or as part of another substituent, means, unless otherwise stated, a fluorine, chlorine, bromine, or iodine atom. Additionally, as the term “haloalkyl” refers to an alkyl group as defined herein, that is substituted with one or more halogen(s) (e.g., 1-3 halogen(s)). For example, the term “C₁-C₄haloalkyl” is meant to include trifluoromethyl, difluoromethyl, 2,2,2-trifluoroethyl, 4-chlorobutyl, 3-bromopropyl, and the like.

The term “hydroxyalkyl” refers to an alkyl group, as defined herein, that is substituted with one or more hydroxyl groups (e.g., 1-3 hydroxyl groups). Exemplary hydroxyalkyl groups include methanol, ethanol, 1,2-propanediol, 1,2-hexanediol, glycerol, and the like.

The compounds of the present disclosure can be present in their neutral form, or as a pharmaceutically acceptable salt, isomer, polymorph or solvate thereof, and may be present in a crystalline form, amorphous form or mixtures thereof.

As referred to herein, “pharmaceutically acceptable salt” is meant to include salts of the compounds according to this disclosure that are prepared with suitably nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from suitably nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present disclosure may contain both basic and acidic functionalities that allow the compounds to be converted into either base or acid addition salts.

The neutral forms of the compounds may be regenerated by contacting the salt with a base or acid and isolating the parent compound.

This disclosure also contemplates isomers of the compounds described herein (e.g., stereoisomers, and atropisomers). For example, certain compounds of the present disclosure possess asymmetric carbon atoms (chiral centers); or hindered rotation about a single bond; the racemates, diastereomers, enantiomers, and atropisomers (e.g., Rₐ, Sₐ, P and M isomers) of which are all intended to be encompassed within the scope of the present disclosure. Stereoisomeric forms may be defined, in terms of absolute stereochemistry, as (R) or (S), and/or depicted uses dashes and/or wedges. When a stereochemical depiction (e.g., using dashes, [symbol] , and/or wedges, [symbol] ) is shown in a chemical structure, or a stereochemical assignment (e.g., using (R) and (S) notation) is made in a chemical name, it is meant to indicate that the depicted isomer is present and substantially free of one or more other isomer(s) (e.g., enantiomers and diastereomers, when present). “Substantially free of” other isomer(s) indicates at least an 70/30 ratio of the indicated isomer to the other isomer(s), more preferably 80/20, 90/10, or 95/5 or more. In some embodiments, the indicated isomer will be present in an amount of at least 99%. A chemical bond to an asymmetric carbon that is depicted as a solid line ( [symbol] ) or a wavy line ( [symbol] ) indicates that all possible stereoisomers (e.g., enantiomers, diastereomers, racemic mixtures, etc.) at that carbon atom are included. In such instances, the compound may be present as a racemic mixture, scalemic mixture, or a mixture of diastereomers.

The compounds of the present disclosure may also contain unnatural proportions of atomic isotopes at one or more of the atoms that constitute such compounds. Unnatural proportions of an isotope may be defined as ranging from the amount found in nature to an amount consisting of 100% of the atom in question. For example, the compounds may incorporate radioactive isotopes, such as for example tritium (³H), iodine-125 (¹²⁵I) or carbon-14 (¹⁴C), or non-radioactive isotopes, such as deuterium (²H) or carbon-13 (¹³C). Such isotopic variations can provide additional utilities to those described elsewhere herein. For instance, isotopic variants of the compounds of the disclosure may find additional utility, including but not limited to, as diagnostic and/or imaging reagents, or as cytotoxic/radiotoxic therapeutic agents. Additionally, isotopic variants of the compounds of the disclosure can have altered pharmacokinetic and pharmacodynamic characteristics which can contribute to enhanced safety, tolerability or efficacy during treatment. In some embodiments, the compounds according to this disclosure are characterized by one or more deuterium atoms.

The terms “treat”, “treating”, treatment” and the like refer to a course of action that eliminates, reduces, suppresses, mitigates, ameliorates, or prevents the worsening of, either temporarily or permanently, a disease, disorder or condition to which the term applies, or at least one of the symptoms associated therewith. Treatment includes alleviation of symptoms, diminishment of extent of disease, inhibiting (e.g., arresting the development or further development of the disease, disorder or condition or clinical symptoms association therewith) an active disease, delaying or slowing of disease progression, improving the quality of life, and/or prolonging survival of a subject as compared to expected survival if not receiving treatment or as compared to a published standard of care therapy for a particular disease.

The term “in need of treatment” as used herein refers to a judgment made by a physician or similar professional that a subject requires or will benefit from treatment. This judgment is made based on a variety of factors that are in the realm of the physician's expertise, which may include a positive diagnosis of a disease, disorder or condition.

The terms “prevent”, “preventing”, “prevention”, “prophylaxis” and the like refer to a course of action initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state. Prevention also refers to a course of action initiated in a subject after the subject has been treated for a disease, disorder, condition or a symptom associated therewith in order to prevent relapse of that disease, disorder, condition or symptom.

The term “in need of prevention” as used herein refers to a judgment made by a physician or other caregiver that a subject requires or will benefit from preventative care. This judgment is made based on a variety of factors that are in the realm of a physician's or caregiver's expertise.

“Substantially pure” indicates that a component (e.g., a compound according to this disclosure) makes up greater than about 50% of the total content of the composition, and typically greater than about 60% of the total content. More typically, “substantially pure” refers to compositions in which at least 75%, at least 85%, at least 90% or more of the total composition is the component of interest. In some cases, the component of interest will make up greater than about 90%, or greater than about 95% of the total content of the composition.

Compounds that are selective may be particularly useful in the treatment of certain disorders or may offer a reduced likelihood of undesired side effects.

Compounds provided herein may have advantageous pharmacokinetic profiles including, for example, metabolic liabilities, permeability, bioavailability, low efflux, hepatocyte stability, clearance, inhibition against CYP, and/or inhibition against hERG.

### Compounds of the Disclosure

In one aspect, this disclosure is directed to a compound having a structure according to Formula I:

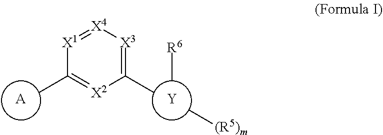

or a pharmaceutically acceptable salt thereof, wherein:
A has a formula selected from the group consisting of:

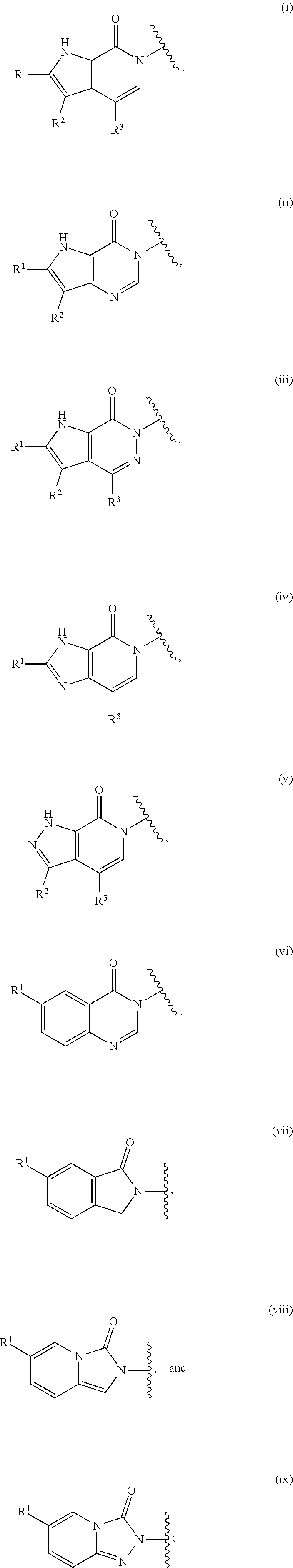

wherein:
R¹ is selected from the group consisting of —H, —C₁-C₆ alkyl, —C₁-C₆ haloalkyl, —C₁-C₆ hydroxyalkyl, -(Q¹)-O—(C₁-C₃ alkyl), —C(O)NH₂, —C(O)—(C₁-C₆-alkyl), -(Q¹)-NR^{1a}R^{1b},
-(Q¹)-(C₃-C₇ cycloalkyl), -(Q¹)-(5- to 6-membered heteroaryl), and -(Q¹)-(4- to 8-membered heterocycloalkyl); wherein said 5- to 6-membered heteroaryl has 1-3 ring heteroatoms independently selected from N, O, and S; said 4- to 8-membered heterocycloalkyl has 1-3 ring heteroatom or heteroatom groups independently selected from N, O, S, and S(O)₂; said C₁-C₃ alkyl is unsubstituted or substituted with —C₁-C₃ alkoxy; and said C₃-C₇ cycloalkyl, 4- to 8-membered heterocycloalkyl, and 5- to 6-membered heteroaryl are unsubstituted or substituted with 1-2 substituents independently selected from halo, —OH, —C₁-C₃ alkyl, —C₁-C₃ alkoxy, and —C(O)(C₁-C₃ alkyl);
Q¹ is absent, unsubstituted —(C₁-C₃ alkylene)-, or —(C₁-C₃ alkylene)- substituted with 1-3 R^{q};
each R^{q} is independently halo, —OH, or —NH₂;
R^{1a} and R^{1b} are independently selected from the group consisting of —H, —C₁-C₆ alkyl, —C₁-C₆ haloalkyl, phenyl, —(C₁-C₃ alkylene)-O—(C₁-C₃ alkyl), —C₃-C₆ cycloalkyl, —(C₁-C₃ alkylene)-(C₃-C₆ cycloalkyl), —S(O)₂(C₁-C₆ alkyl), 5- to 6-membered heteroaryl having 1-3 ring heteroatoms independently selected from N, O, and S, and 4- to 8-membered heterocycloalkyl having 1-3 ring heteroatom or heteroatom groups independently selected from N, O, S, and S(O)₂; wherein said phenyl, —(C₁-C₃ alkylene)-O—(C₁-C₃ alkyl), —C₃-C₆ cycloalkyl, —(C₁-C₃ alkylene)-(C₃-C₆ cycloalkyl), 5- to 6-membered heteroaryl, and 4- to 8-membered heterocycloalkyl are unsubstituted or substituted with 1-3 R^{1c};
each R^{1c}, when present, is independently halo, —OH, —C₁-C₃ alkyl, —C₁-C₃ hydroxyalkyl, or —C₁-C₃ haloalkyl;
R², when present, is —H, halo, —CN, —C₁-C₃ alkyl, —C₁-C₃ haloalkyl, —C₃-C₄ cycloalkyl,
—S(O)₂(C₁-C₃ alkyl), —C(O)—NR^{2a}R^{2b}, or 5- to 6-membered heteroaryl having 1-3 ring heteroatoms independently selected from N, O, and S; or
R¹ and R² taken together with the atoms to which they are attached form —C₃-C₆ cycloalkyl;
R^{2a} and R^{2b} are independently —H, or —C₁-C₃ alkyl;
R³, when present, is —H, —CN, halo, —C₁-C₆ alkyl, —C₁-C₆ haloalkyl, —C₁-C₆ hydroxyalkyl, —C₂-C₃ alkenyl, —C₃-C₄ cycloalkyl, —S(O)₂(C₁-C₆ alkyl), —C(O)OH, or 5- to 6-membered heteroaryl having 1 to 4 ring heteroatoms independently selected from N, S, and O; and the 5- to 6-membered heteroaryl is unsubstituted or substituted with 1-3 substituents independently selected from —C₁-C₃ alkyl;
X¹, X² and X³ are each independently N or CH;
X⁴ is N or CR⁴;
R⁴, when present, is —H, halo, —CN, —OH, —C₁-C₆ alkyl, —C₁-C₆ haloalkyl, —C₁-C₆ alkoxy, —NR^{4a}R^{4b} or —C₃-C₈ cycloalkyl;
R^{4a} and R^{4b} are independently —H, —C₁-C₃ alkyl, or —(C₁-C₃ alkylene)-NR^{4c}R^{4d};
R^{4c} and R^{4d} are independently —H, or —C₁-C₃ alkyl;
Y is phenyl, or 5- to 6-membered heteroaryl having 1-3 ring heteroatoms independently selected from N, O, and S;
m is 0, 1, 2, or 3;
each R⁵ when present, is independently halo, —CN, —C₁-C₆ alkyl, —C₁-C₆ haloalkyl, or —C₁-C₆ alkoxy;
when ring A has a structure of formula (i), (ii), (iii), (iv), (v), or (vi), R⁶ is a 5- to 6-membered heteroaryl having 1 to 4 ring heteroatoms independently selected from N, O, and S, wherein R⁶ is unsubstituted or substituted with 1-3 R^{6a}; or
when ring A has a structure of formula (vii), (viii), or (ix), R⁶ is a 6-membered heteroaryl having 1 to 4 ring heteroatoms independently selected from N, O, and S, wherein R⁶ is unsubstituted or substituted with 1-3 R^{6a}; and
each R^{6a} is independently selected from the group consisting of —CN, —C₁-C₃ alkyl, —C₁-C₃ haloalkyl, and —C₁-C₃ hydroxyalkyl.

In some embodiments, the compound has a structure according to Formula I:

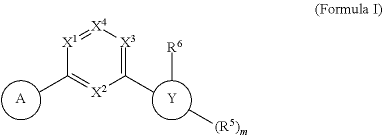

or a pharmaceutically acceptable salt thereof, wherein:
A has a formula selected from the group consisting of:

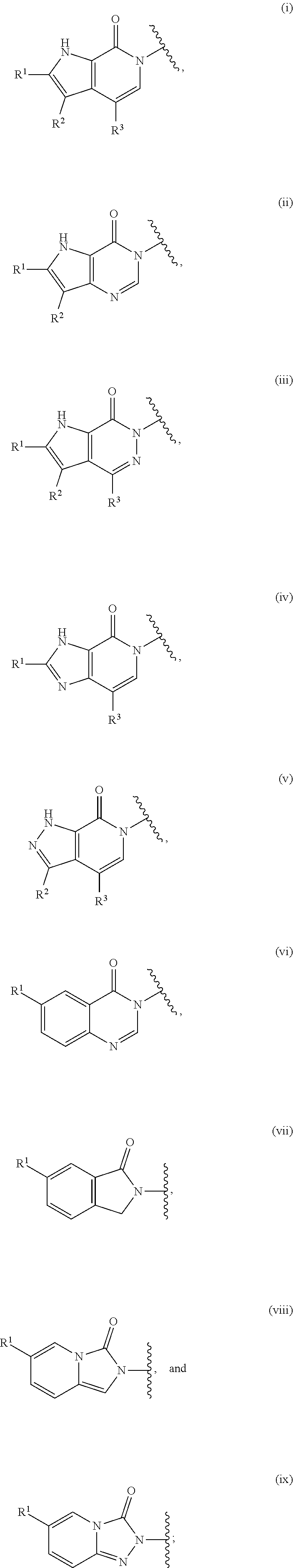

wherein:
R¹ is selected from the group consisting of —H, —C₁-C₆ haloalkyl, —C₁-C₆ hydroxyalkyl, —C(O)NH₂, —C(O)—(C₁-C₆-alkyl), -(Q¹)-NR^{1a}R^{1b}, -(Q¹)-(C₃-C₇ cycloalkyl), -(Q¹)-(5- to 6-membered heteroaryl), and -(Q¹)-(4- to 8-membered heterocycloalkyl); wherein said 5- to 6-membered heteroaryl has 1-3 ring heteroatoms independently selected from N, O, and S; said 4- to 8-membered heterocycloalkyl has 1-3 ring heteroatom or heteroatom groups independently selected from N, O, S, and S(O)₂; and said C₃-C₇ cycloalkyl, 4- to 8-membered heterocycloalkyl, and 5- to 6-membered heteroaryl are unsubstituted or substituted with 1-2 substituents independently selected from halo, —OH, —C₁-C₃ alkyl, and —C₁-C₃ alkoxy;
Q¹ is absent, unsubstituted —(C₁-C₃ alkylene)-, or —(C₁-C₃ alkylene)- substituted with 1-3 R^{q};
each R^{q} is independently halo, —OH, or —NH₂;
R^{1a} and R^{1b} are independently selected from the group consisting of —H, —C₁-C₆ alkyl, —C₁-C₆ haloalkyl, phenyl, —(C₁-C₃ alkylene)-O—(C₁-C₃ alkyl), —C₃-C₆ cycloalkyl, —(C₁-C₃ alkylene)-(C₃-C₆ cycloalkyl), —S(O)₂(C₁-C₆ alkyl), 5- to 6-membered heteroaryl having 1-3 ring heteroatoms independently selected from N, O, and S, and 4- to 8-membered heterocycloalkyl having 1-3 ring heteroatom or heteroatom groups independently selected from N, O, S, and S(O)₂; wherein said phenyl, —(C₁-C₃ alkylene)-O—(C₁-C₃ alkyl), —C₃-C₆ cycloalkyl, —(C₁-C₃ alkylene)-(C₃-C₆ cycloalkyl), 5- to 6-membered heteroaryl, and 4- to 8-membered heterocycloalkyl are unsubstituted or substituted with 1-3 R^{1c};
each R^{1c}, when present, is independently halo, —OH, —C₁-C₃ alkyl, —C₁-C₃ hydroxyalkyl, or —C₁-C₃ haloalkyl;
R², when present, is —H, halo, —CN, —C₁-C₃ alkyl, —C₁-C₃ haloalkyl, —C₃-C₄ cycloalkyl, —S(O)₂(C₁-C₃ alkyl), —C(O)—NR^{2a}R^{2b}, or 5- to 6-membered heteroaryl having 1-3 ring heteroatoms independently selected from N, O, and S;
R^{2a} and R^{2b} are independently —H, or —C₁-C₃ alkyl;
R³, when present, is —H, —CN, halo, —C₁-C₆ alkyl, —C₁-C₆ haloalkyl, —C₁-C₆ hydroxyalkyl, —C₂-C₃ alkenyl, —C₃-C₄ cycloalkyl, —S(O)₂(C₁-C₆ alkyl), —C(O)OH, or 5- to 6-membered heteroaryl having 1 to 4 ring heteroatoms independently selected from N, S, and O; and the 5- to 6-membered heteroaryl is unsubstituted or substituted with 1-3 substituents independently selected from —C₁-C₃ alkyl;
X¹, X² and X³ are each independently N or CH;
X⁴ is N or CR⁴;
R⁴, when present, is —H, halo, —CN, —OH, —C₁-C₆ alkyl, —C₁-C₆ haloalkyl, —C₁-C₆ alkoxy, —NR^{4a}R^{4b} or —C₃-C₈ cycloalkyl;
R^{4a} and R^{4b} are independently —H, —C₁-C₃ alkyl, or —(C₁-C₃ alkylene)-NR^{4c}R^{4d};
R^{4c} and R^{4d} are independently —H, or —C₁-C₃ alkyl;
Y is phenyl, or 5- to 6-membered heteroaryl having 1-3 ring heteroatoms independently selected from N, O, and S;
m is 0, 1, 2, or 3;
each R⁵ when present, is independently halo, —CN, —C₁-C₆ alkyl, —C₁-C₆ haloalkyl, or —C₁-C₆ alkoxy;
when ring A has a structure of formula (i), (ii), (iii), (iv), (v), or (vi), R⁶ is a 5- to 6-membered heteroaryl having 1 to 4 ring heteroatoms independently selected from N, O, and S, wherein R⁶ is unsubstituted or substituted with 1-3 R^{6a}; or
when ring A has a structure of formula (vii), (viii), or (ix), R⁶ is a 6-membered heteroaryl having 1 to 4 ring heteroatoms independently selected from N, O, and S, wherein R⁶ is unsubstituted or substituted with 1-3 R^{6a}; and
each R^{6a} is independently selected from the group consisting of —C₁-C₃ alkyl, —C₁-C₃ haloalkyl, and —C₁-C₃ hydroxyalkyl.

In some embodiments, R¹ is selected from the group consisting of —H, —C₁-C₆ alkyl, —C₁-C₆ haloalkyl, —C₁-C₆ hydroxyalkyl, -(Q¹)-O—(C₁-C₃ alkyl), -(Q¹)-NR^{1a}R^{1b}, -(Q¹)-(C₃-C₇ cycloalkyl), -(Q¹)-(5- to 6-membered heteroaryl), and -(Q¹)-(4- to 8-membered heterocycloalkyl); wherein said 5- to 6-membered heteroaryl has 1-3 ring heteroatoms independently selected from N, O, and S; said 4- to 8-membered heterocycloalkyl has 1-3 ring heteroatom or heteroatom groups independently selected from N, O, S, and S(O)₂; said C₁-C₃ alkyl is unsubstituted or substituted with —C₁-C₃ alkoxy; and said C₃-C₇ cycloalkyl, 4- to 8-membered heterocycloalkyl, and 5- to 6-membered heteroaryl are unsubstituted or substituted with 1-2 substituents independently selected from halo, —OH, —C₁-C₃ alkyl, —C₁-C₃ alkoxy, and —C(O)(C₁-C₃ alkyl); Q¹ is absent, unsubstituted —(C₁-C₃ alkylene)- or —(C₁-C₃ alkylene)- substituted with 1-3 R^{q}; each R^{q} is independently halo, —OH, or —NH₂; R^{1a} and R^{1b} are independently selected from the group consisting of —H, —C₁-C₆ alkyl, —C₁-C₆ haloalkyl, —(C₁-C₃ alkylene)-O—(C₁-C₃ alkyl), —C₃-C₆ cycloalkyl, —(C₁-C₃ alkylene)-(C₃-C₆ cycloalkyl), and 4- to 8-membered heterocycloalkyl having 1-3 ring heteroatoms independently selected from N, O, and S; wherein said —(C₁-C₃ alkylene)-O—(C₁-C₃ alkyl), —C₃-C₆ cycloalkyl, —(C₁-C₃ alkylene)-(C₃-C₆ cycloalkyl), and 4- to 8-membered heterocycloalkyl are unsubstituted or substituted with 1-3 R^{1c}; and each R^{1c}, when present, is independently halo, —OH, —C₁-C₃ alkyl, or —C₁-C₃ hydroxyalkyl.

In some embodiments, R¹ is selected from the group consisting of —H, —C₁-C₆ hydroxyalkyl, —C(O)NH₂, —C(O)—(C₁-C₆-alkyl), -(Q¹)-NR^{1a}R^{1b}, and -(Q¹)-(4- to 8-membered heterocycloalkyl); wherein said 4- to 8-membered heterocycloalkyl has 1-3 ring heteroatom or heteroatom groups independently selected from N, O, S, and S(O)₂; and said 4- to 8-membered heterocycloalkyl is unsubstituted or substituted with 1-2 substituents independently selected from halo, —OH, —C₁-C₃ alkyl, and —C₁-C₃ alkoxy; Q¹ is unsubstituted —(C₁-C₃ alkylene)-; Ria and R^{1b} are independently selected from the group consisting of —H, —C₁-C₆ alkyl, —C₁-C₆ haloalkyl, —C₃-C₆ cycloalkyl, —(C₁-C₃ alkylene)-(C₃-C₆ cycloalkyl), and 4- to 8-membered heterocycloalkyl having 1-3 ring heteroatom or heteroatom groups independently selected from N, O, and S; wherein said —C₃-C₆ cycloalkyl, —(C₁-C₃ alkylene)-(C₃-C₆ cycloalkyl), and 4- to 8-membered heterocycloalkyl are unsubstituted or substituted with 1-3 R^{1c}; and each R^{1c}, when present, is independently —OH, —C₁-C₃ alkyl, or —C₁-C₃ hydroxyalkyl.

In some embodiments, R¹ is —C₁-C₆ hydroxyalkyl, -(Q¹)-NR^{1a}R^{1b} or -(Q¹)-(4- to 8-membered heterocycloalkyl) having 1-3 ring heteroatom or heteroatom groups independently selected from N, O, S, and S(O)₂; and said 4- to 8-membered heterocycloalkyl is unsubstituted or substituted with 1-2 substituents independently selected from halo, —OH, —C₁-C₃ alkyl, and —C₁-C₃ alkoxy.

In some embodiments, R¹ is selected from the group consisting of —H, -(Q¹)-NR^{1a}R^{1b} and -(Q¹)-(4- to 8-membered heterocycloalkyl) having 1-3 ring heteroatom or heteroatom groups independently selected from N, O, S, and S(O)₂; and said 4- to 8-membered heterocycloalkyl is unsubstituted or substituted with 1-2 substituents independently selected from halo, —OH, —C₁-C₃ alkyl, and —C₁-C₃ alkoxy; R^{1a} and R^{1b} are independently —H, or —(C₁-C₃ alkylene)-(C₃-C₆ cycloalkyl), wherein said —(C₁-C₃ alkylene)-(C₃-C₆ cycloalkyl) is unsubstituted or substituted with 1 R^{1c}; and R^{1c}, when present, is —OH.

In some embodiments, R¹ is selected from -(Q¹)-NR^{1a}R^{1b} and -(Q¹)-(4- to 8-membered heterocycloalkyl) having 1-3 ring heteroatoms independently selected from N, O, and S; and R^{1a} and R^{1b} are independently —H, unsubstituted —(C₁-C₃ alkylene)-(C₃-C₆ cycloalkyl), or —(C₁-C₃ alkylene)-(C₃-C₆ cycloalkyl) substituted with 1 R^{1c}.

In some embodiments, R¹ is H. In some embodiments, R¹ is —C₁-C₆ alkyl. In some embodiments, R¹ is —C₁-C₆ haloalkyl. In some embodiments, R¹ is —C₁-C₆ hydroxyalkyl. In some embodiments, R¹ is -(Q¹)-O—(C₁-C₃ alkyl), wherein said C₁-C₃ alkyl is unsubstituted or substituted with —C₁-C₃ alkoxy. In some embodiments, R¹ is —C(O)NH₂. In some embodiments, R¹ is —C(O)—(C₁-C₆-alkyl). In some embodiments, R¹ is -(Q¹)-NR^{1a}R^{1b}. In some embodiments, R¹ is -(Q¹)-(C₃-C₇ cycloalkyl), wherein said C₃-C₇ cycloalkyl is unsubstituted or substituted with 1-2 substituents independently selected from halo, —OH, —C₁-C₃ alkyl, —C₁-C₃ alkoxy, and —C(O)(C₁-C₃ alkyl). In some embodiments, R¹ is -(Q¹)-(C₃-C₇ cycloalkyl), wherein said C₃-C₇ cycloalkyl is unsubstituted or substituted with 1-2 substituents independently selected from halo, —OH, —C₁-C₃ alkyl, and —C₁-C₃ alkoxy. In some embodiments, R¹ is -(Q¹)-(5- to 6-membered heteroaryl) having 1-3 ring heteroatoms independently selected from N, O, and S, wherein said 5- to 6-membered heteroaryl is unsubstituted or substituted with 1-2 substituents independently selected from halo, —OH, —C₁-C₃ alkyl, —C₁-C₃ alkoxy, and —C(O)(C₁-C₃ alkyl). In some embodiments, R¹ is -(Q¹)-(5- to 6-membered heteroaryl) having 1-3 ring heteroatoms independently selected from N, and O, wherein said 5- to 6-membered heteroaryl is unsubstituted or substituted with 1-2 substituents independently selected from halo, —OH, —C₁-C₃ alkyl, —C₁-C₃ alkoxy, and —C(O)(C₁-C₃ alkyl). In some embodiments, R¹ is -(Q¹)-(5- to 6-membered heteroaryl) having 1-3 ring heteroatoms independently selected from N, O, and S, wherein said 5- to 6-membered heteroaryl is unsubstituted or substituted with 1-2 substituents independently selected from halo, —OH, —C₁-C₃ alkyl, and —C₁-C₃ alkoxy. In some embodiments, R¹ is -(Q¹)-(5- to 6-membered heteroaryl) having 1-3 ring heteroatoms independently selected from N, and O, wherein said 5- to 6-membered heteroaryl is unsubstituted or substituted with 1-2 substituents independently selected from halo, —OH, —C₁-C₃ alkyl, and —C₁-C₃ alkoxy. In some embodiments, R¹ is -(Q¹)-(4- to 8-membered heterocycloalkyl) having 1-3 ring heteroatom or heteroatom groups independently selected from N, O, S, and S(O)₂, wherein said 4- to 8-membered heterocycloalkyl is unsubstituted or substituted with 1-2 substituents independently selected from halo, —OH, —C₁-C₃ alkyl, —C₁-C₃ alkoxy, and —C(O)(C₁-C₃ alkyl). In some embodiments, R¹ is -(Q¹)-(4- to 8-membered heterocycloalkyl) having 1-3 ring heteroatom or heteroatom groups independently selected from N, and O, wherein said 4- to 8-membered heterocycloalkyl is unsubstituted or substituted with 1-2 substituents independently selected from halo, —OH, —C₁-C₃ alkyl, —C₁-C₃ alkoxy, and —C(O)(C₁-C₃ alkyl). In some embodiments, R¹ is -(Q¹)-(4- to 8-membered heterocycloalkyl) having 1-3 ring heteroatom or heteroatom groups independently selected from N, O, S, and S(O)₂, wherein said 4- to 8-membered heterocycloalkyl is unsubstituted or substituted with 1-2 substituents independently selected from halo, —OH, —C₁-C₃ alkyl, and —C₁-C₃ alkoxy. In some embodiments, R¹ is -(Q¹)-(4- to 8-membered heterocycloalkyl) having 1-3 ring heteroatom or heteroatom groups independently selected from N, and O, wherein said 4- to 8-membered heterocycloalkyl is unsubstituted or substituted with 1-2 substituents independently selected from halo, —OH, —C₁-C₃ alkyl, and —C₁-C₃ alkoxy.

In some embodiments, Q¹ is absent. In some embodiments, Q¹ is unsubstituted —(C₁-C₃ alkylene)-. In some embodiments, Q¹ is —CH₂—. In some embodiments, Q¹ is —(C₁-C₃ alkylene)- substituted with 1-3 R^{q}.

In some embodiments, R¹ is -(Q¹)-O—(C₁-C₃ alkyl), wherein said C₁-C₃ alkyl is unsubstituted or substituted with —C₁-C₃ alkoxy, and Q¹ is unsubstituted —(C₁-C₃ alkylene)-. In some embodiments, R¹ is -(Q¹)-NR^{1a}R^{1b}, and Q¹ is unsubstituted —(C₁-C₃ alkylene)- or —(C₁-C₃ alkylene)- substituted with 1-3 halo. In some embodiments, R¹ is -(Q¹)-(C₃-C₇ cycloalkyl), wherein said C₃-C₇ cycloalkyl is unsubstituted or substituted with 1 —OH; Q¹ is absent, unsubstituted —(C₁-C₃ alkylene)-, or —(C₁-C₃ alkylene)- substituted with 1-3 R^{q}; and each R^{q} is independently halo or —OH. In some embodiments, R¹ is -(Q¹)-(5- to 6-membered heteroaryl) having 1-3 ring heteroatoms independently selected from N, O, and S, wherein said 5- to 6-membered heteroaryl is unsubstituted or substituted with 1-2 substituents independently selected from —C₁-C₃ alkyl; Q¹ is —(C₁-C₃ alkylene)- substituted with 1-3 R^{q}; and each R^{q} is independently —OH, or —NH₂. In some embodiments, R¹ is -(Q¹)-(4- to 8-membered heterocycloalkyl) having 1-3 ring heteroatom or heteroatom groups independently selected from N, O, S, and S(O)₂, wherein said 4- to 8-membered heterocycloalkyl is unsubstituted or substituted with 1-2 substituents independently selected from halo, —OH, —C₁-C₃ alkyl, —C₁-C₃ alkoxy, and —C(O)(C₁-C₃ alkyl); Q¹ is absent, unsubstituted —(C₁-C₃ alkylene)-, or —(C₁-C₃ alkylene)- substituted with 1 —OH.

In some embodiments, R¹ is -(Q¹)-NR^{1a}R^{1b}; Q¹ is unsubstituted —(C₁-C₃ alkylene)- or —(C₁-C₃ alkylene)- substituted with 1-3 halo; R^{1a} and R^{1b} are independently selected from the group consisting of —H, —C₁-C₆ alkyl, —C₁-C₆ haloalkyl, —(C₁-C₃ alkylene)-O—(C₁-C₃ alkyl), —C₃-C₆ cycloalkyl, —(C₁-C₃ alkylene)-(C₃-C₆ cycloalkyl), and 4- to 8-membered heterocycloalkyl having 1-3 ring heteroatom or heteroatom groups independently selected from N, O, S, and S(O)₂; wherein said —(C₁-C₃ alkylene)-O—(C₁-C₃ alkyl), —C₃-C₆ cycloalkyl, —(C₁-C₃ alkylene)-(C₃-C₆ cycloalkyl), and 4- to 8-membered heterocycloalkyl are unsubstituted or substituted with 1-3 R^{1c}; and each R^{1c}, when present, is independently halo, —OH, —C₁-C₃ alkyl, or —C₁-C₃ hydroxyalkyl.

In some embodiments, R^{1a} and R^{1b} are independently selected from the group consisting of —H, —C₁-C₆ alkyl, —C₁-C₆ haloalkyl, —C₃-C₆ cycloalkyl, —(C₁-C₃ alkylene)-(C₃-C₆ cycloalkyl), and 4- to 8-membered heterocycloalkyl having 1-3 ring heteroatom or heteroatom groups independently selected from N, O, and S; wherein said —C₃-C₆ cycloalkyl, —(C₁-C₃ alkylene)-(C₃-C₆ cycloalkyl), and 4- to 8-membered heterocycloalkyl are unsubstituted or substituted with 1-3 R^{1c}.

In some embodiments, R¹ is —H, —CH₃, —CHF₂, —CH₂OH,

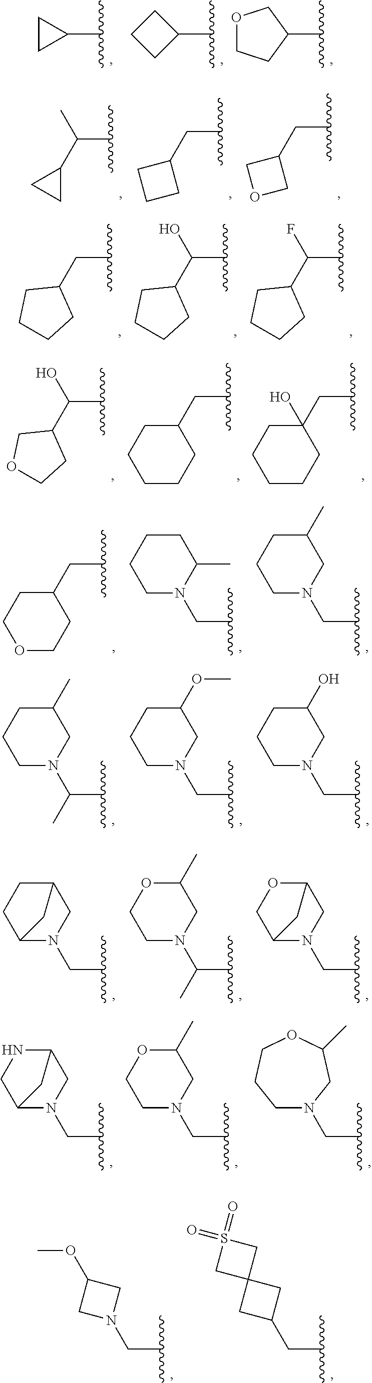

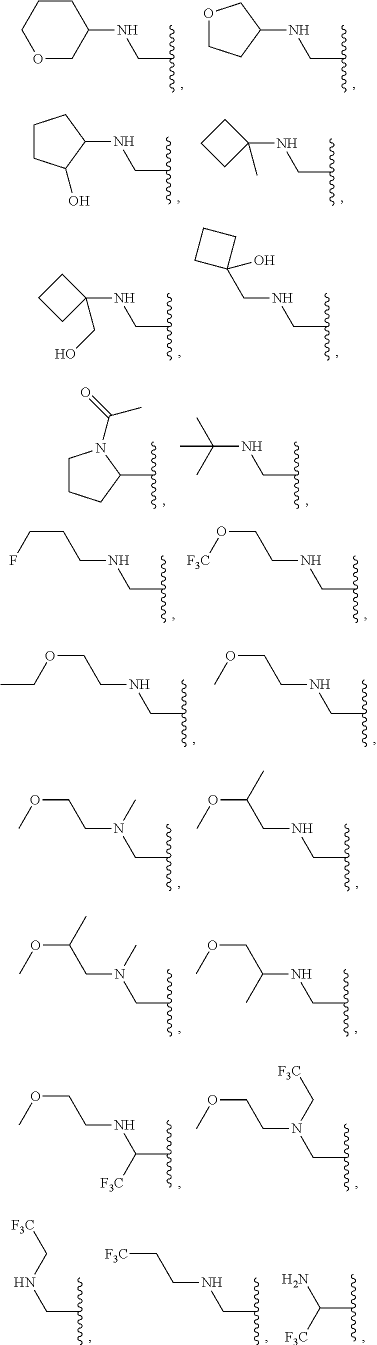

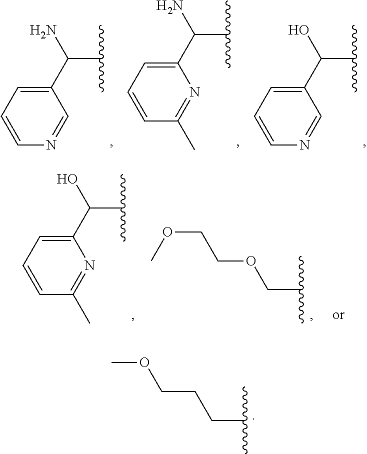

In some embodiments, R¹ is —H, —CH₃, —CHF₂, CH₂OH

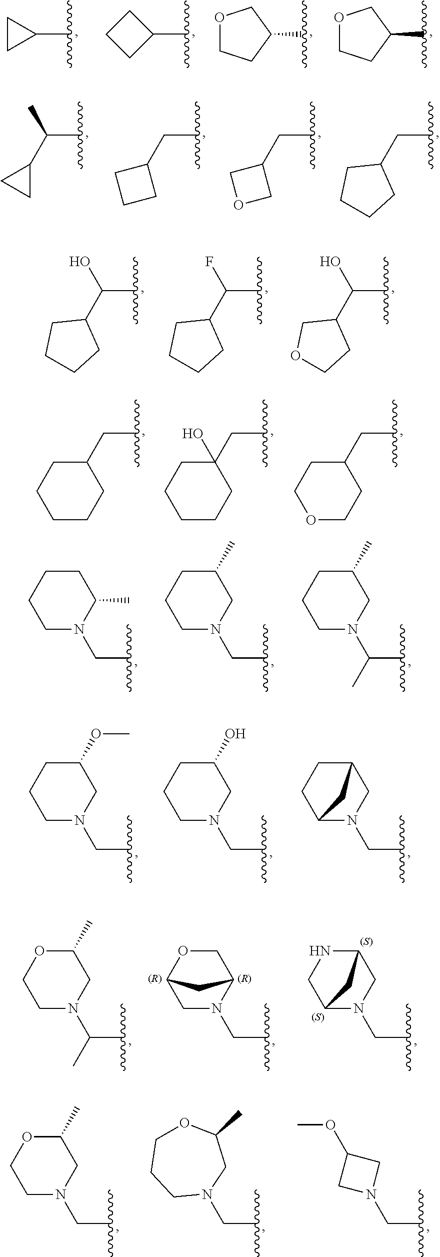

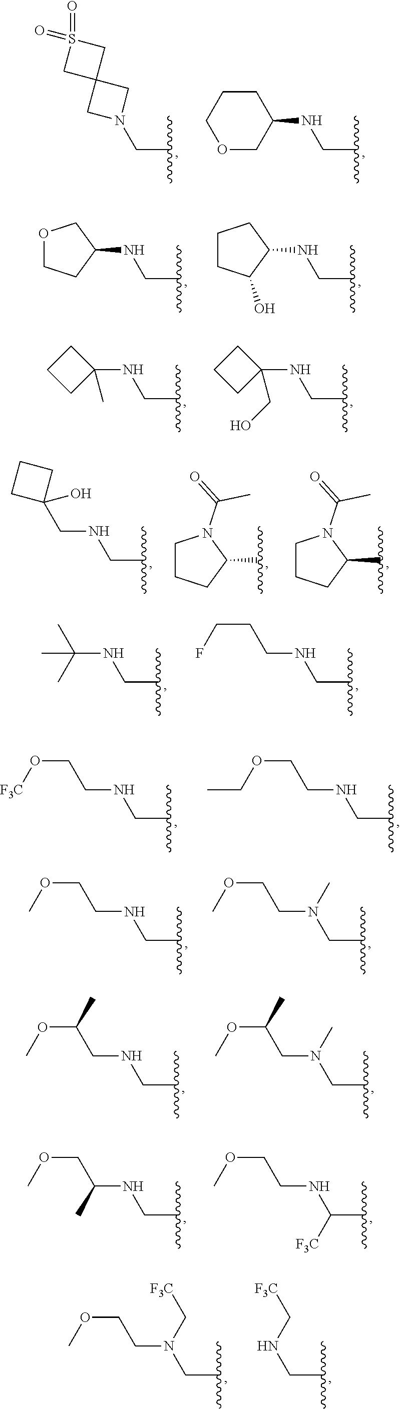

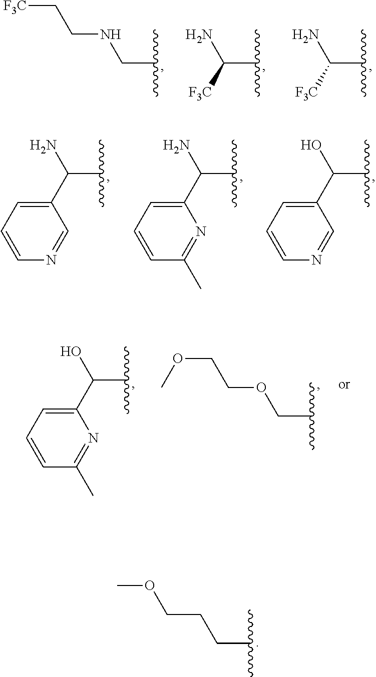

In some embodiments, R¹ is —H,

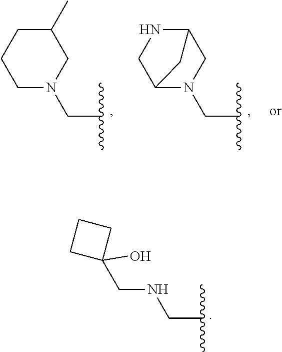

or In some embodiments, R¹ is —H,

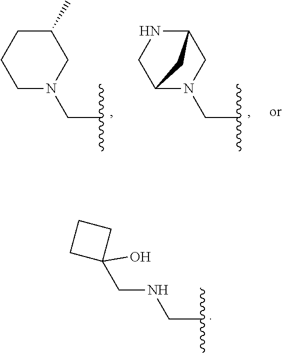

In some embodiments, R¹ is

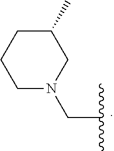 [SMILES: *CN1CCC[C@H](C)C1]

In some embodiments, R², when present is —H. In some embodiments, R², when present, is halo. In some embodiments, R², when present, is —CN. In some embodiments, R², when present, is —C₁-C₃ alkyl. In some embodiments, R², when present, is —C₁-C₃ haloalkyl. In some embodiments, R², when present, is —C₃-C₄ cycloalkyl. In some embodiments, R², when present, is —S(O)₂(C₁-C₃ alkyl). In some embodiments, R², when present, is —C(O)—NR^{2a}R^{2b}, wherein R^{2a} and R^{2b} are independently —H or —C₁-C₃ alkyl. In some embodiments, R², when present, is a 5- to 6-membered heteroaryl having 1-3 ring heteroatoms independently selected from N, O, and S. In some embodiments, R², when present, is a 5- to 6-membered heteroaryl having 1-3 ring heteroatoms independently selected from N and O.

In some embodiments, R², when present is —H or —CN.

In some embodiments, R¹ and R² taken together with the atoms to which they are attached form —C₃-C₆ cycloalkyl. In some embodiments, R¹ and R² taken together with the atoms to which they are attached form —C₅-C₆ cycloalkyl.

In some embodiments, R³, when present, is —CN, —C₁-C₆ alkyl, —C₁-C₆ haloalkyl, —C₃-C₄ cycloalkyl, or —S(O)₂(C₁-C₆ alkyl). In some embodiments, R³, when present, is —CN, —C₁-C₆ haloalkyl, —C₃-C₄ cycloalkyl, or —S(O)₂(C₁-C₆ alkyl). In some embodiments, R³, when present, is —CN, methyl, trifluoromethyl, cyclopropyl, or —S(O)₂CH₃. In some embodiments, R³, when present, is —CN, trifluoromethyl, cyclopropyl, or —S(O)₂CH₃. In some embodiments, R³, when present, is cyclopropyl.

In some embodiments, R³, when present, is —H. In some embodiments, R³, when present, is —CN. In some embodiments, R³, when present, is halo. In some embodiments, R³, when present, is —C₁-C₆ alkyl. In some embodiments, R³, when present, is —C₁-C₆ haloalkyl. In some embodiments, R³, when present, is —C₁-C₆ hydroxyalkyl. In some embodiments, R³, when present, is —C₂-C₃ alkenyl. In some embodiments, R³, when present, is —C₃-C₄ cycloalkyl. In some embodiments, R³, when present, is —S(O)₂(C₁-C₆ alkyl). In some embodiments, R³, when present, is —C(O)OH. In some embodiments, R³, when present, is a 5- to 6-membered heteroaryl having 1 to 4 ring heteroatoms independently selected from N, S, and O; and the 5- to 6-membered heteroaryl is unsubstituted or substituted with 1-3 substituents independently selected from —C₁-C₃ alkyl.

In some embodiments, R³, when present, is —C₁-C₆ haloalkyl or —C₃-C₄ cycloalkyl.

In some embodiments, X¹ is N, X² is CH, and X³ is CH. In some embodiments, X¹, X², and X³ are CH.

In some embodiments, X¹ is N, X² is CH, X³ is CH, and X⁴ is CR⁴. In some embodiments, X¹, X², and X³ are CH, and X⁴ is CR⁴. In some embodiments, X¹, X², X³, and X⁴ are CH.

In some embodiments, the compound has a structure of Formula Ia:

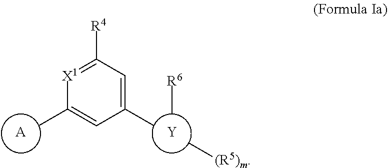

In some embodiments, R⁴ is —H, halo, —CN, —C₁-C₆ alkyl, —C₁-C₆ haloalkyl, —C₁-C₆ alkoxy, —NR^{4a}R^{4b}, or —C₃-C₈ cycloalkyl; and R^{4a} and R^{4b} are independently —H or —C₁-C₃ alkyl.

In some embodiments, R⁴ is H, halo, —OH, —C₁-C₆ haloalkyl, —C₁-C₆ alkoxy, —NR^{4a}R^{4b}, or —C₃-C₈ cycloalkyl.

In some embodiments, R⁴ is —H, halo, —C₁-C₆ haloalkyl, —C₁-C₆ alkoxy, —NR^{4a}R^{4b}, or —C₃-C₈ cycloalkyl; and R^{4a} and R^{4b} are independently —H or —C₁-C₃ alkyl.

In some embodiments, R⁴, when present, is —H. In some embodiments, R⁴, when present, is halo. In some embodiments, R⁴, when present, is —CN. In some embodiments, R⁴, when present, is —OH. In some embodiments, R⁴, when present, is C₁-C₆ alkyl. In some embodiments, R⁴, when present, is C₁-C₆ haloalkyl. In some embodiments, R⁴, when present, is —C₁-C₆ alkoxy. In some embodiments, R⁴, when present, is —NR^{4a}R^{4b}. In some embodiments, R⁴, when present, is —C₃-C₈ cycloalkyl.

In some embodiments, R⁴, when present, is —H, —CN, —F, —Cl, —CH₃, —CF₃, —OH, —OCH₃, —OCH₂CH₃,

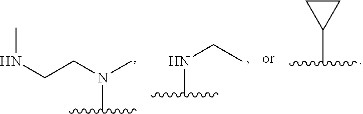

In some embodiments, R⁴, when present, is —H, —Cl, —OH, —CF₃, —OCH₃,

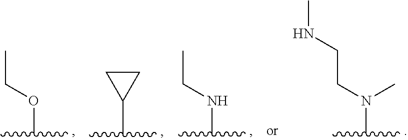

In some embodiments, R⁴, when present, is

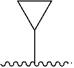 [SMILES: *C1CC1]

In some embodiments, Y is phenyl, or a 5-membered heteroaryl having 1-3 ring heteroatoms independently selected from N, O, and S. In some embodiments, Y is phenyl, or a 5-membered heteroaryl having 1-3 ring N heteroatoms. In some embodiments, Y is phenyl or pyrazole. In some embodiments, Y is phenyl. In some embodiments, Y is pyrazole.

In some embodiments, the compound has a structure of Formula Ib or Formula Ic:

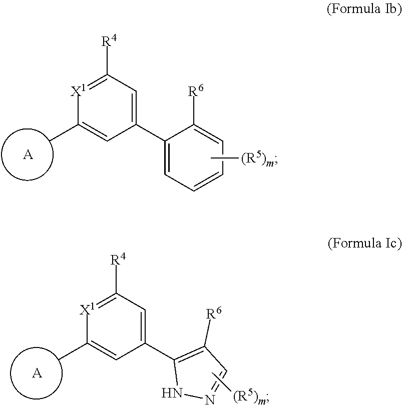

wherein m is 0, 1, or 2. In some embodiments X¹ is N or CH. In some embodiments, X¹ is N. In further embodiments, X¹ is CH.

In some embodiments, the compound of Formula Ib has a structure of Formula Ib-1:

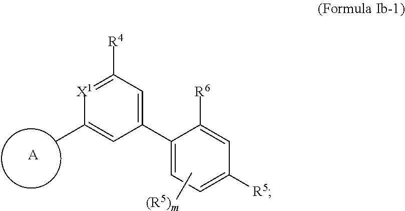

wherein m is 0 or 1. In some embodiments X¹ is N or CH. In some embodiments, X¹ is N. In further embodiments, X¹ is CH.

In some embodiments, the compound of Formula Ic has a structure of Formula Ic-1:

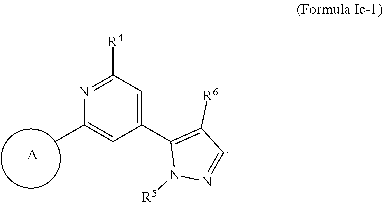

In some embodiments, each R⁵, when present, is independently halo, —CN, —C₁-C₆ alkyl, or —C₁-C₆ alkoxy. In some embodiments, each R⁵, when present, is independently halo, —CN, —C₁-C₃ alkyl, —C₁-C₃ haloalkyl or —C₁-C₃ alkoxy. In some embodiments, each R⁵, when present, is independently —F, —CN, —CH₃, —CF₃, or —OCH₃. In some embodiments, each R⁵, when present, is independently —F, —CN, —CH₃, or —OCH₃.

In some embodiments, R⁵ is halo. In some embodiments, R⁵ is —CN. In some embodiments, R⁵ is —C₁-C₆ alkyl. In some embodiments, R⁵ is —C₁-C₃ alkyl. In some embodiments, R⁵ is —C₁-C₆ haloalkyl. In some embodiments, R⁵ is —C₁-C₃ haloalkyl. In some embodiments, R⁵ is —C₁-C₆ alkoxy. In some embodiments, R⁵ is —C₁-C₃ alkoxy. In some embodiments, each R⁵, when present, is independently —F, —CN, —CH₃, —CF₃, or —OCH₃.

In some embodiments, R⁶ is a 5- to 6-membered heteroaryl having 1 to 4 ring heteroatoms independently selected from N, O, and S, wherein R⁶ is unsubstituted or substituted with 1-3 R^{6a}. In some embodiments, R⁶ is a 5- to 6-membered heteroaryl having 1 to 3 ring heteroatoms independently selected from N, and O, wherein R⁶ is unsubstituted or substituted with 1-3 R^{6a}. In some embodiments, R⁶ is triazole, imidazole, pyrazole, oxazole, pyridine, pyridazine, or pyrimidine, each of which is unsubstituted, or substituted with 1-2 R^{6a}. In some embodiments, R⁶ is triazole, imidazole, pyrazole, pyridine, pyridazine, or pyrimidine, each of which is unsubstituted, or substituted with 1-2 R^{6a}. In some embodiments, R⁶ is triazole, imidazole, or pyrazole, each of which is unsubstituted, or substituted with 1-2 R^{6a}.

In some embodiments, R⁶ is

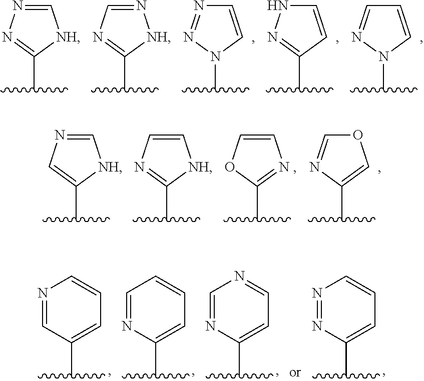

each of which is unsubstituted, or substituted with 1-2 R^{6a}. In some embodiments, R⁶ is

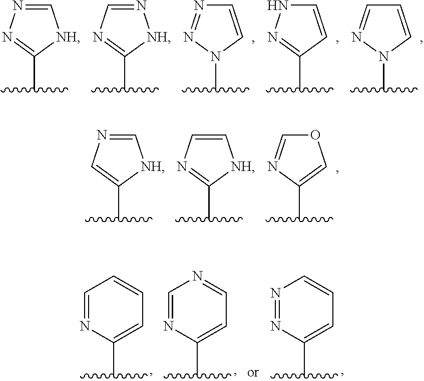

each of which is unsubstituted, or substituted with 1-2 R^{6a}. In some embodiments, R⁶ is

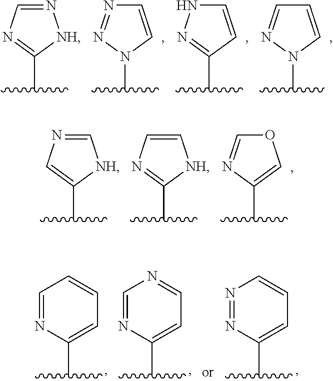

each of which is unsubstituted, or substituted with 1-2 R^{6a}. In some embodiments, R⁶ is

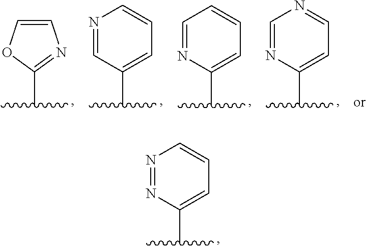

each of which is unsubstituted, or substituted with 1-2 R^{6a}. In some embodiments, R⁶ is

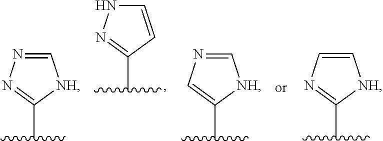

each of which is unsubstituted, or substituted with 1-2 R^{6a}.

In some embodiments, R⁶ is

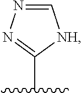 [SMILES: *c1nnc[nH]1]

which is unsubstituted, or substituted with 1-2 R^{6a}. In some embodiments, R⁶ is

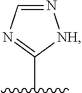 [SMILES: *c1ncn[nH]1]

which is unsubstituted, or substituted with 1-2 R^{6a}. In some embodiments, R⁶ is

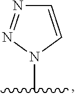 [SMILES: *n1ccnn1]

which is unsubstituted, or substituted with 1-2 R^{6a}. In some embodiments, R⁶ is

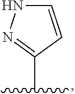 [SMILES: *c1cc[nH]n1]

which is unsubstituted, or substituted with 1-2 R^{6a}. In some embodiments, R⁶ is

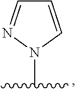 [SMILES: *n1cccn1]

which is unsubstituted, or substituted with 1-2 R^{6a}. In some embodiments, R⁶ is

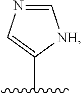 [SMILES: *c1cnc[nH]1]

which is unsubstituted, or substituted with 1-2 R^{6a}. In some embodiments, R⁶ is

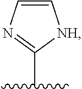 [SMILES: *c1ncc[nH]1]

which is unsubstituted, or substituted with 1-2 R^{6a}. In some embodiments, R⁶ is

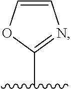 [SMILES: *c1ncco1]

which is unsubstituted, or substituted with 1-2 R^{6a}. In some embodiments, R⁶ is

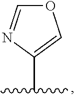 [SMILES: *c1cocn1]

which is unsubstituted, or substituted with 1-2 R^{6a}. In some embodiments, R⁶ is

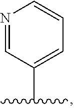 [SMILES: *c1cccnc1]

which is unsubstituted, or substituted with 1-2 R^{6a}. In some embodiments, R⁶ is

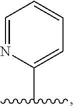 [SMILES: *c1ccccn1]

which is unsubstituted, or substituted with 1-2 R^{6a}. In some embodiments, R⁶ is

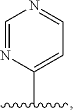 [SMILES: *c1ccncn1]

which is unsubstituted, or substituted with 1-2 R^{6a}. In some embodiments, R⁶ is

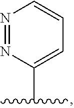 [SMILES: *c1cccnn1]

which is unsubstituted, or substituted with 1-2 R^{6a}.

In some embodiments, R^{6a} is —CN, —C₁-C₃ alkyl, or —C₁-C₃ haloalkyl. In some embodiments, R^{6a} is —C₁-C₃ alkyl, or —C₁-C₃ haloalkyl. In some embodiments, R^{6a} is —CH₃, —CH₂CH₃, —CF₂H, —CF₃, or —CH₂CH₂F. In some embodiments, R^{6a} is —CH₃, —CH₂CH₃, —CF₂H, or —CF₃.

In some embodiments, A is

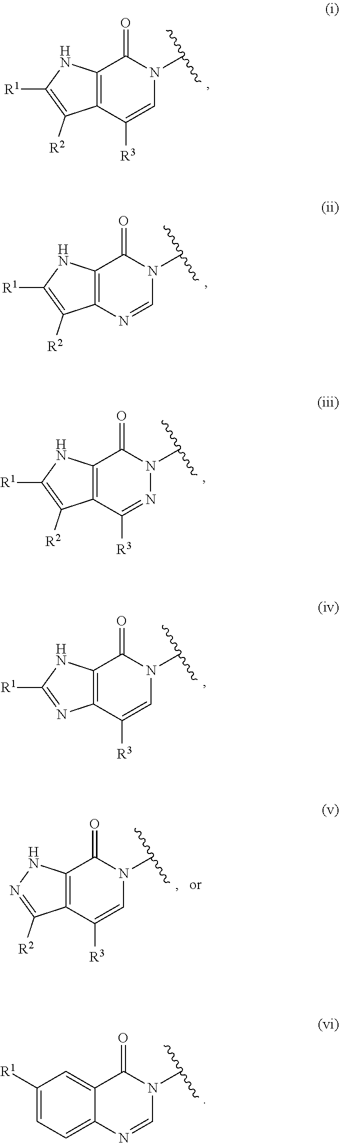

In some embodiments, A is

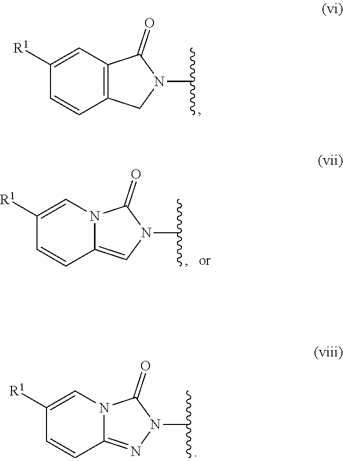

In some embodiments, A is:

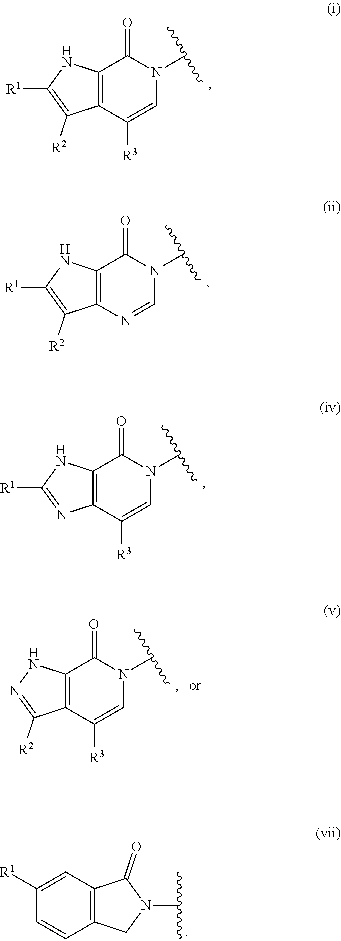

In some embodiments, A is:

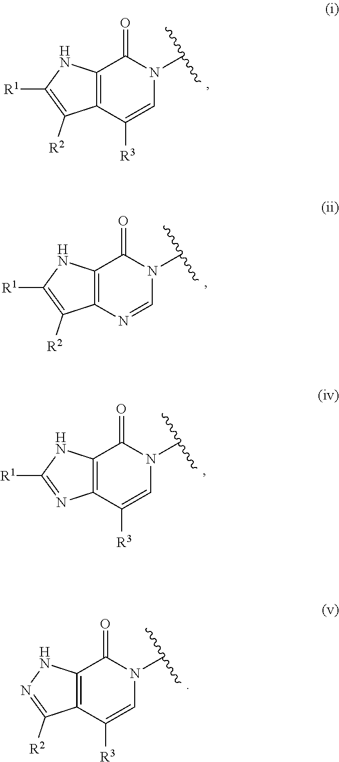

In some embodiments, A is:

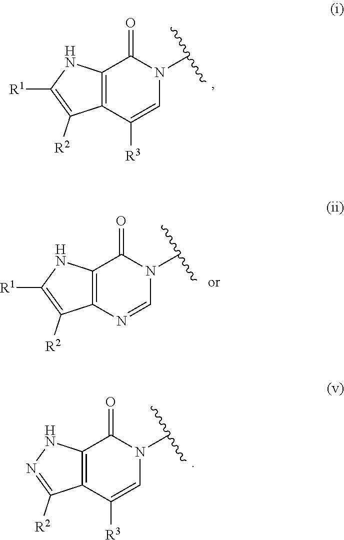

In some embodiments, A is

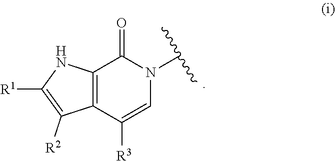

In some embodiments, A is

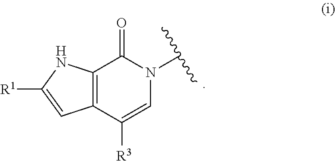

In some embodiments, A is

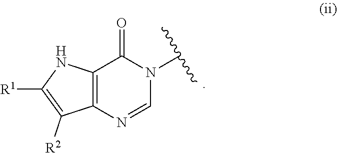

In some embodiments, A is

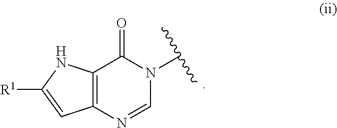

In some embodiments, A is

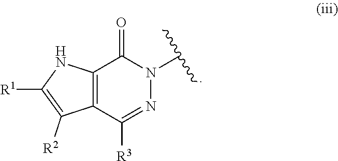

In some embodiments, A is

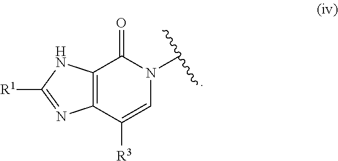

In some embodiments, A is

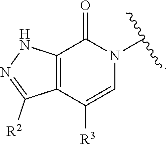

In some embodiments, A is

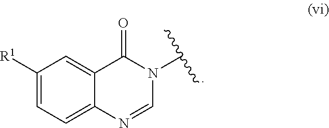

In some embodiments, A is

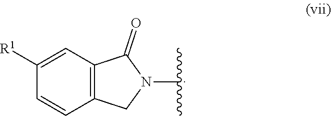

In some embodiments, A is

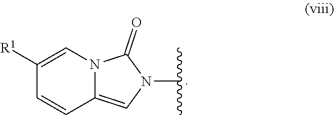

In some embodiments, A is

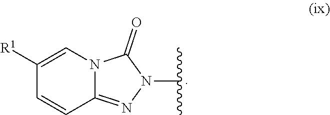

In some embodiments, this disclosure is directed to a compound of Formula Ia:

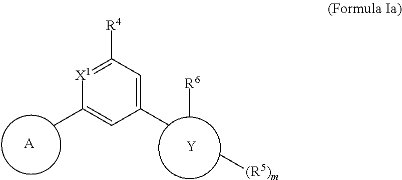

or a pharmaceutically acceptable salt thereof, wherein:
A has a formula selected from the group consisting of:

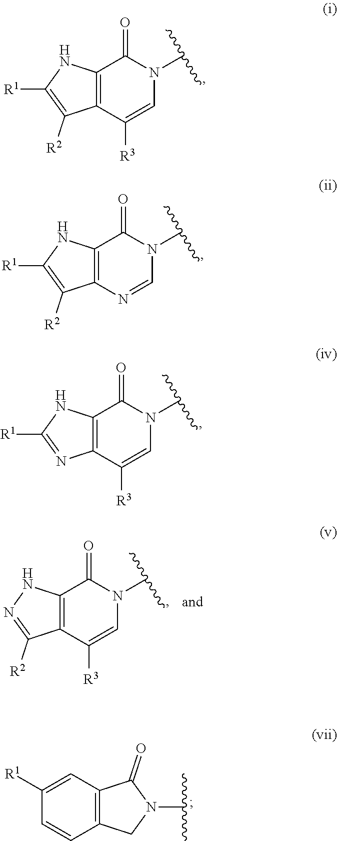

wherein:
R¹ is selected from the group consisting of —H, —C₁-C₆ hydroxyalkyl, —C(O)NH₂, —C(O)—(C₁-C₆-alkyl), -(Q¹)-NR^{1a}R^{1b}, and -(Q¹)-(4- to 8-membered heterocycloalkyl); wherein said 4- to 8-membered heterocycloalkyl has 1-3 ring heteroatom or heteroatom groups independently selected from N, O, S, and S(O)₂; and said 4- to 8-membered heterocycloalkyl is unsubstituted or substituted with 1-2 substituents independently selected from halo, —OH, —C₁-C₃ alkyl, and —C₁-C₃ alkoxy;
Q¹ is unsubstituted —(C₁-C₃ alkylene)-;
R^{1a} and R^{1b} are independently selected from the group consisting of —H, —C₁-C₆ alkyl, —C₁-C₆ haloalkyl, —C₃-C₆ cycloalkyl, —(C₁-C₃ alkylene)-(C₃-C₆ cycloalkyl), and 4- to 8-membered heterocycloalkyl having 1-3 ring heteroatom or heteroatom groups independently selected from N, O, and S; wherein said —C₃-C₆ cycloalkyl, —(C₁-C₃ alkylene)-(C₃-C₆ cycloalkyl), and 4- to 8-membered heterocycloalkyl are unsubstituted or substituted with 1-3 R^{1c}; and
each R^{1c}, when present, is independently —OH, —C₁-C₃ alkyl, or —C₁-C₃ hydroxyalkyl;
R² is H or —CN;
R³, when present, is —CN, —C₁-C₆ alkyl, —C₁-C₆ haloalkyl, —C₃-C₄ cycloalkyl, or —S(O)₂(C₁-C₆ alkyl);
X¹ is CH or N;
R⁴, when present, is —H, halo, —CN, —OH, —C₁-C₆ alkyl, —C₁-C₆ haloalkyl, —C₁-C₆ alkoxy, —NR^{4a}R^{4b} or —C₃-C₈ cycloalkyl;
R^{4a} and R^{4b} are independently —H, —C₁-C₃ alkyl, or —(C₁-C₃ alkylene)-NR^{4c}R^{4d};
R^{4c} and R^{4d} are independently —H, or —C₁-C₃ alkyl;
Y is phenyl, or a 5-membered heteroaryl having 1-3 ring nitrogen atoms;
m is 0, 1, 2, or 3;
each R⁵ when present, is independently halo, —CN, —C₁-C₆ alkyl, —C₁-C₆ haloalkyl, or —C₁-C₆ alkoxy;
when ring A has a structure of formula (i), (ii), (iv), or (v), R⁶ is a 5- to 6-membered heteroaryl having 1 to 4 heteroatoms independently selected from N, O, and S, wherein R⁶ is unsubstituted or substituted with 1-3 R^{6a}; or
when ring A has a structure of formula (vii), R⁶ is a 6-membered heteroaryl having 1 to 4 heteroatoms independently selected from N, O, and S, wherein R⁶ is unsubstituted or substituted with 1-3 R^{6a}; and
each R^{6a} is independently selected from the group consisting of —C₁-C₃ alkyl, —C₁-C₃ haloalkyl, and —C₁-C₃ hydroxyalkyl.

In some embodiments, this disclosure is directed to a compound of Formula Ia:

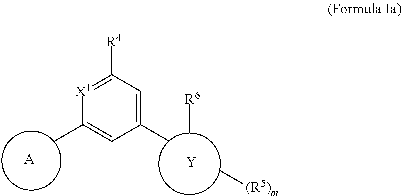

or a pharmaceutically acceptable salt thereof, wherein:
A has a formula selected from the group consisting of:

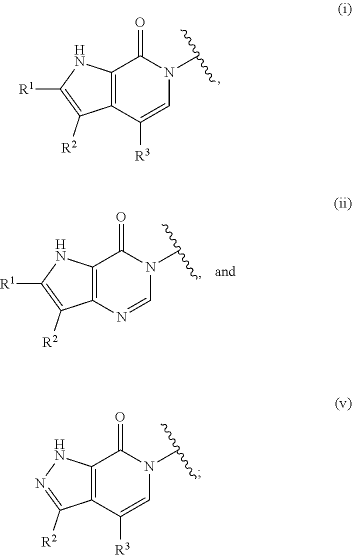

wherein:
R¹ is selected from the group consisting of —H, -(Q¹)-NR^{1a}R^{1b}, and -(Q¹)-(4- to 8-membered heterocycloalkyl) having 1-3 ring heteroatom or heteroatom groups independently selected from N, O, S, and S(O)₂; and said 4- to 8-membered heterocycloalkyl is unsubstituted or substituted with 1-2 substituents independently selected from halo, —OH, —C₁-C₃ alkyl, and —C₁-C₃ alkoxy;
Q¹ is unsubstituted —(C₁-C₃ alkylene)-;
R^{1a} and R^{1b} are independently —H, or —(C₁-C₃ alkylene)-(C₃-C₆ cycloalkyl), wherein said —(C₁-C₃ alkylene)-(C₃-C₆ cycloalkyl) is unsubstituted or substituted with 1 R^{1c}; and R^{1c}, when present, is —OH;
R² is H or —CN;
R³, when present, is —C₁-C₆ haloalkyl or —C₃-C₄ cycloalkyl;
X¹ is CH or N;
R⁴ is H, halo, —OH, —C₁-C₆ haloalkyl, —C₁-C₆ alkoxy, —NR^{4a}R^{4b}, or —C₃-C₈ cycloalkyl;
R^{4a} and R^{4b} are independently —H, —C₁-C₃ alkyl, or —(C₁-C₃ alkylene)-NR^{4c}R^{4d};
R^{4c} and R^{4d} are independently —H, or —C₁-C₃ alkyl;
Y is phenyl, or a 5-membered heteroaryl having 1-3 ring nitrogen atoms;
m is 0, 1, 2, or 3;
each R⁵, when present, is independently halo, —CN, —C₁-C₆ alkyl, or —C₁-C₆ alkoxy;
R⁶ is 5-membered heteroaryl having 1 to 3 ring nitrogen atoms; wherein R⁶ is unsubstituted or substituted with 1-3 R^{6a}; and
each R^{6a} is independently —C₁-C₃ alkyl, or —C₁-C₃ haloalkyl.

In one or more embodiments, the compound, or pharmaceutically acceptable salt or solvate thereof, according to this disclosure is selected from the compounds provided in Table 1 or Table 2. In one or more embodiments, the compound according to this disclosure is selected from the compounds provided in Table 1 or Table 2.

TABLE 1

| Example | Structure |
|---|---|
| 1 | 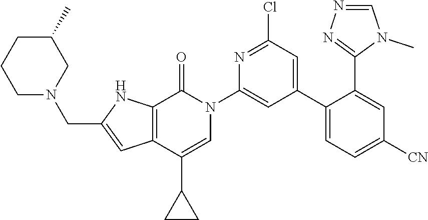 [SMILES: C[C@H]1CCCN(Cc2cc3c(C4CC4)cn(-c4cc(-c5ccc(C#N)cc5-c5nncn5C)cc(Cl)n4)c(=O)c3[nH]2)C1] |
| 2 | 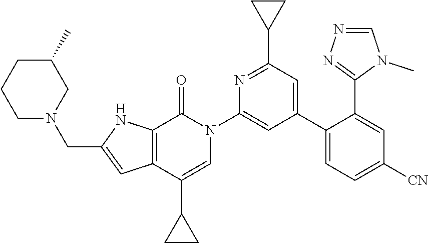 [SMILES: C[C@H]1CCCN(Cc2cc3c(C4CC4)cn(-c4cc(-c5ccc(C#N)cc5-c5nncn5C)cc(C5CC5)n4)c(=O)c3[nH]2)C1] |
| 3 | 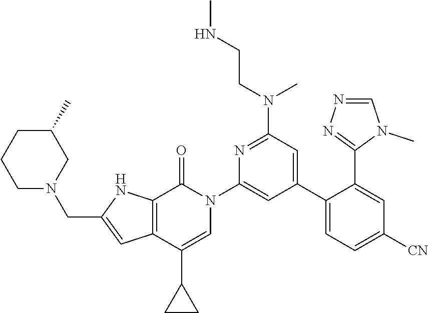 [SMILES: CNCCN(C)c1cc(-c2ccc(C#N)cc2-c2nncn2C)cc(-n2cc(C3CC3)c3cc(CN4CCC[C@H](C)C4)[nH]c3c2=O)n1] |
| 4 | 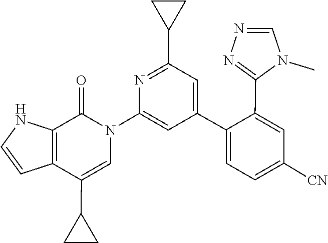 [SMILES: Cn1cnnc1-c1cc(C#N)ccc1-c1cc(C2CC2)nc(-n2cc(C3CC3)c3cc[nH]c3c2=O)c1] |
| 5 | 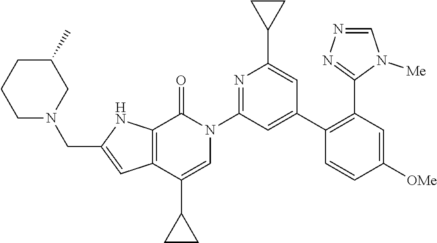 [SMILES: COc1ccc(-c2cc(C3CC3)nc(-n3cc(C4CC4)c4cc(CN5CCC[C@H](C)C5)[nH]c4c3=O)c2)c(-c2nncn2C)c1] |
| 6 | 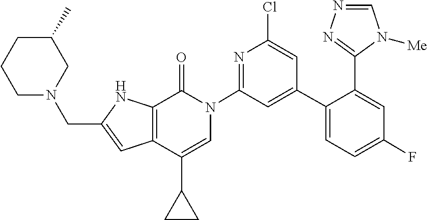 [SMILES: C[C@H]1CCCN(Cc2cc3c(C4CC4)cn(-c4cc(-c5ccc(F)cc5-c5nncn5C)cc(Cl)n4)c(=O)c3[nH]2)C1] |
| 7 | 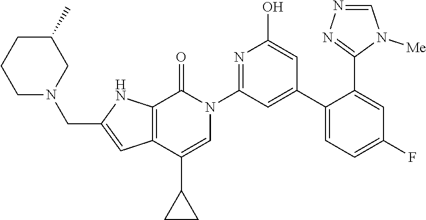 [SMILES: C[C@H]1CCCN(Cc2cc3c(C4CC4)cn(-c4cc(-c5ccc(F)cc5-c5nncn5C)cc(O)n4)c(=O)c3[nH]2)C1] |
| 8 | 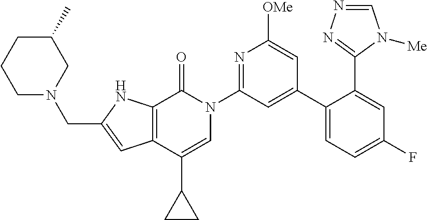 [SMILES: COc1cc(-c2ccc(F)cc2-c2nncn2C)cc(-n2cc(C3CC3)c3cc(CN4CCC[C@H](C)C4)[nH]c3c2=O)n1] |
| 9 | 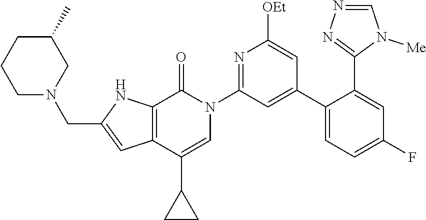 [SMILES: CCOc1cc(-c2ccc(F)cc2-c2nncn2C)cc(-n2cc(C3CC3)c3cc(CN4CCC[C@H](C)C4)[nH]c3c2=O)n1] |
| 10 | 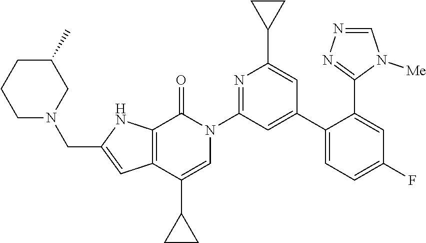 [SMILES: C[C@H]1CCCN(Cc2cc3c(C4CC4)cn(-c4cc(-c5ccc(F)cc5-c5nncn5C)cc(C5CC5)n4)c(=O)c3[nH]2)C1] |
| 11 | 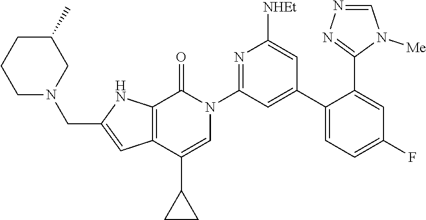 |
| 12 | 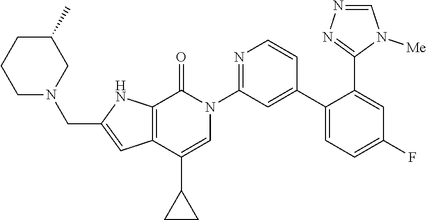 [SMILES: C[C@H]1CCCN(Cc2cc3c(C4CC4)cn(-c4cc(-c5ccc(F)cc5-c5nncn5C)ccn4)c(=O)c3[nH]2)C1] |
| 13 | 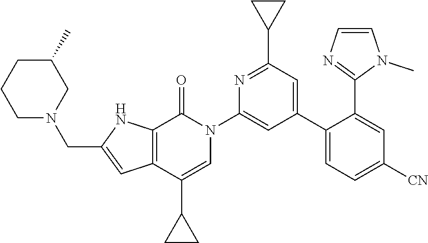 [SMILES: C[C@H]1CCCN(Cc2cc3c(C4CC4)cn(-c4cc(-c5ccc(C#N)cc5-c5nccn5C)cc(C5CC5)n4)c(=O)c3[nH]2)C1] |
| 14 | 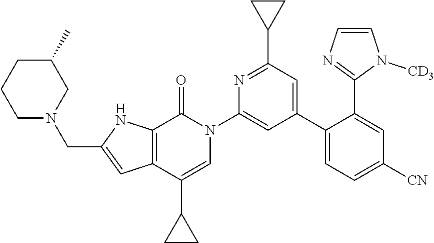 [SMILES: [2H]C([2H])([2H])n1ccnc1-c1cc(C#N)ccc1-c1cc(C2CC2)nc(-n2cc(C3CC3)c3cc(CN4CCC[C@H](C)C4)[nH]c3c2=O)c1] |
| 15 | 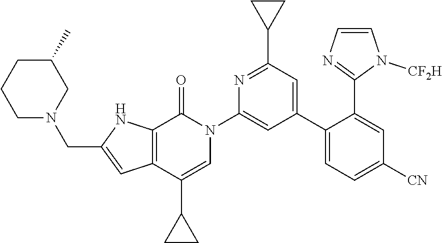 [SMILES: C[C@H]1CCCN(Cc2cc3c(C4CC4)cn(-c4cc(-c5ccc(C#N)cc5-c5nccn5C(F)F)cc(C5CC5)n4)c(=O)c3[nH]2)C1] |
| 16 | 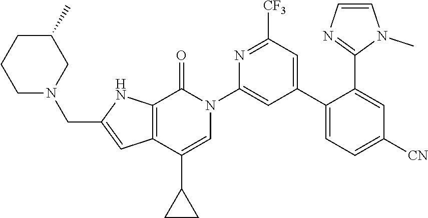 [SMILES: C[C@H]1CCCN(Cc2cc3c(C4CC4)cn(-c4cc(-c5ccc(C#N)cc5-c5nccn5C)cc(C(F)(F)F)n4)c(=O)c3[nH]2)C1] |
| 17 | 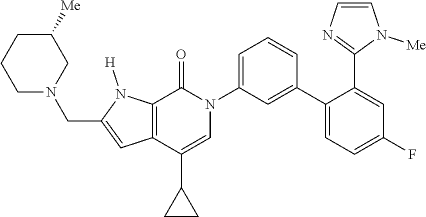 [SMILES: C[C@H]1CCCN(Cc2cc3c(C4CC4)cn(-c4cccc(-c5ccc(F)cc5-c5nccn5C)c4)c(=O)c3[nH]2)C1] |
| 18 | 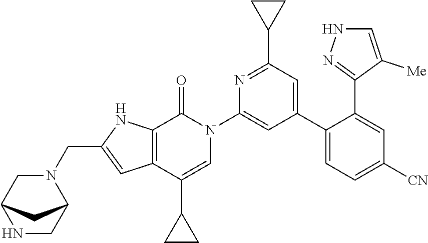 [SMILES: Cc1c[nH]nc1-c1cc(C#N)ccc1-c1cc(C2CC2)nc(-n2cc(C3CC3)c3cc(CN4C[C@@H]5C[C@H]4CN5)[nH]c3c2=O)c1] |
| 19 | 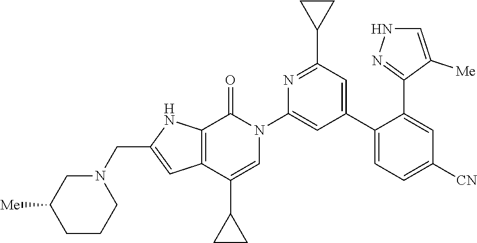 [SMILES: Cc1c[nH]nc1-c1cc(C#N)ccc1-c1cc(C2CC2)nc(-n2cc(C3CC3)c3cc(CN4CCC[C@H](C)C4)[nH]c3c2=O)c1] |
| 20 | 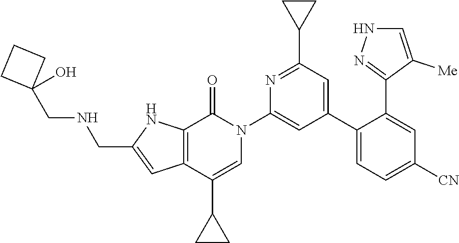 [SMILES: Cc1c[nH]nc1-c1cc(C#N)ccc1-c1cc(C2CC2)nc(-n2cc(C3CC3)c3cc(CNCC4(O)CCC4)[nH]c3c2=O)c1] |
| 21 | 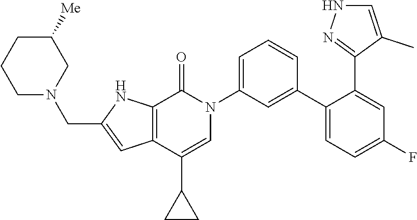 [SMILES: Cc1c[nH]nc1-c1cc(F)ccc1-c1cccc(-n2cc(C3CC3)c3cc(CN4CCC[C@H](C)C4)[nH]c3c2=O)c1] |
| 22 | 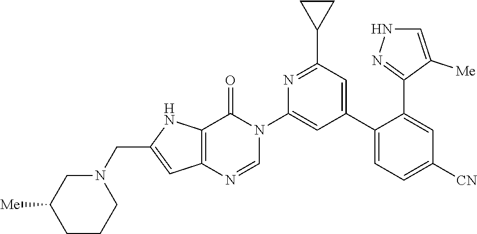 [SMILES: Cc1c[nH]nc1-c1cc(C#N)ccc1-c1cc(C2CC2)nc(-n2cnc3cc(CN4CCC[C@H](C)C4)[nH]c3c2=O)c1] |
| 23 | 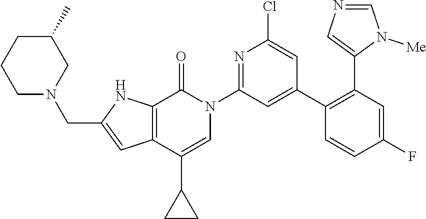 [SMILES: C[C@H]1CCCN(Cc2cc3c(C4CC4)cn(-c4cc(-c5ccc(F)cc5-c5cncn5C)cc(Cl)n4)c(=O)c3[nH]2)C1] |
| 24 | 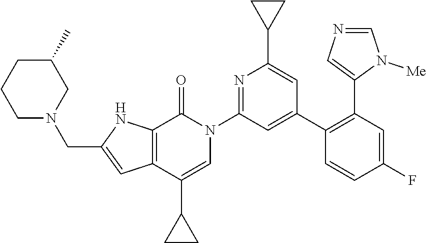 [SMILES: C[C@H]1CCCN(Cc2cc3c(C4CC4)cn(-c4cc(-c5ccc(F)cc5-c5cncn5C)cc(C5CC5)n4)c(=O)c3[nH]2)C1] |
| 25 | 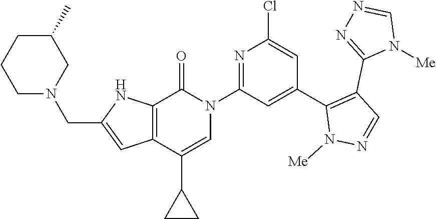 [SMILES: C[C@H]1CCCN(Cc2cc3c(C4CC4)cn(-c4cc(-c5c(-c6nncn6C)cnn5C)cc(Cl)n4)c(=O)c3[nH]2)C1] |
| 26 | 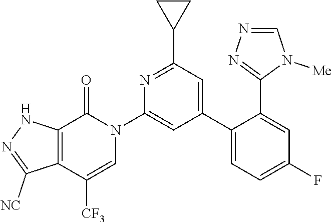 [SMILES: Cn1cnnc1-c1cc(F)ccc1-c1cc(C2CC2)nc(-n2cc(C(F)(F)F)c3c(C#N)n[nH]c3c2=O)c1] |
| 27 | 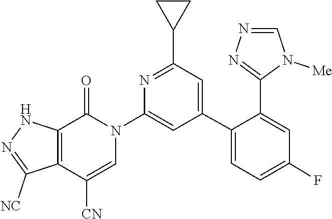 [SMILES: Cn1cnnc1-c1cc(F)ccc1-c1cc(C2CC2)nc(-n2cc(C#N)c3c(C#N)n[nH]c3c2=O)c1] |

TABLE 2

| Example | Structure |
|---|---|
| 28 | 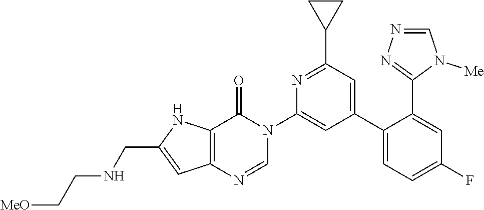 [SMILES: COCCNCc1cc2ncn(-c3cc(-c4ccc(F)cc4-c4nncn4C)cc(C4CC4)n3)c(=O)c2[nH]1] |
| 29 | 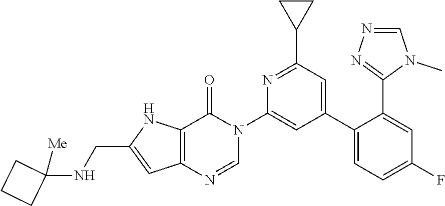 [SMILES: Cn1cnnc1-c1cc(F)ccc1-c1cc(C2CC2)nc(-n2cnc3cc(CNC4(C)CCC4)[nH]c3c2=O)c1] |
| 30 | 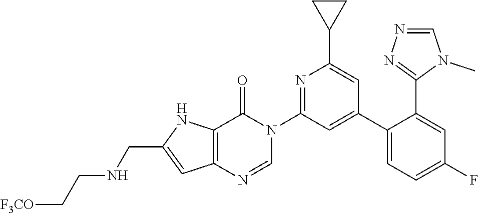 [SMILES: Cn1cnnc1-c1cc(F)ccc1-c1cc(C2CC2)nc(-n2cnc3cc(CNCCOC(F)(F)F)[nH]c3c2=O)c1] |
| 31 | 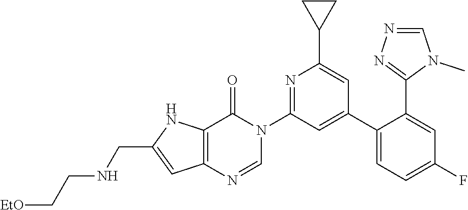 [SMILES: CCOCCNCc1cc2ncn(-c3cc(-c4ccc(F)cc4-c4nncn4C)cc(C4CC4)n3)c(=O)c2[nH]1] |
| 32 | 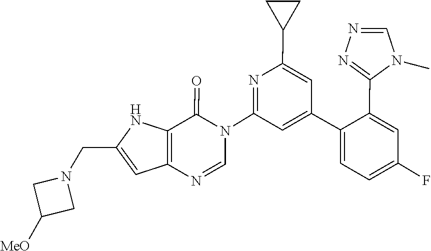 [SMILES: COC1CN(Cc2cc3ncn(-c4cc(-c5ccc(F)cc5-c5nncn5C)cc(C5CC5)n4)c(=O)c3[nH]2)C1] |
| 33 | 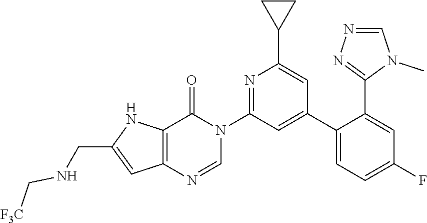 [SMILES: Cn1cnnc1-c1cc(F)ccc1-c1cc(C2CC2)nc(-n2cnc3cc(CNCC(F)(F)F)[nH]c3c2=O)c1] |
| 34 | 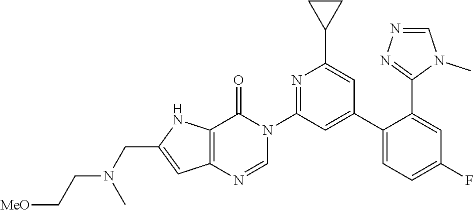 [SMILES: COCCN(C)Cc1cc2ncn(-c3cc(-c4ccc(F)cc4-c4nncn4C)cc(C4CC4)n3)c(=O)c2[nH]1] |
| 35 | 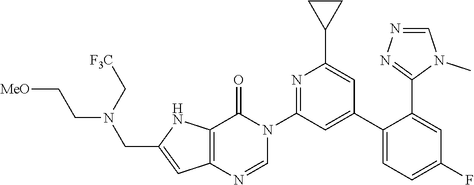 [SMILES: COCCN(Cc1cc2ncn(-c3cc(-c4ccc(F)cc4-c4nncn4C)cc(C4CC4)n3)c(=O)c2[nH]1)CC(F)(F)F] |
| 36 | 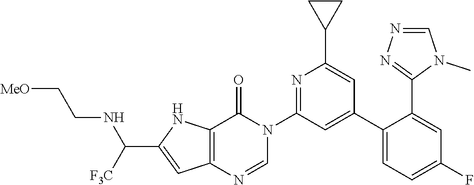 [SMILES: COCCNC(c1cc2ncn(-c3cc(-c4ccc(F)cc4-c4nncn4C)cc(C4CC4)n3)c(=O)c2[nH]1)C(F)(F)F] |
| 37 | 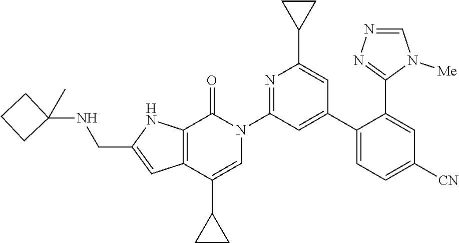 [SMILES: Cn1cnnc1-c1cc(C#N)ccc1-c1cc(C2CC2)nc(-n2cc(C3CC3)c3cc(CNC4(C)CCC4)[nH]c3c2=O)c1] |
| 38 | 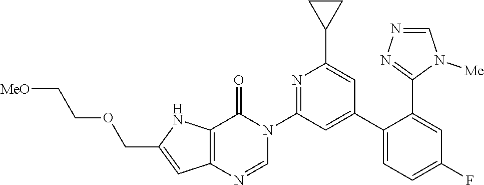 [SMILES: COCCOCc1cc2ncn(-c3cc(-c4ccc(F)cc4-c4nncn4C)cc(C4CC4)n3)c(=O)c2[nH]1] |
| 39 | 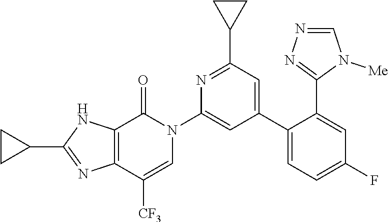 [SMILES: Cn1cnnc1-c1cc(F)ccc1-c1cc(C2CC2)nc(-n2cc(C(F)(F)F)c3nc(C4CC4)[nH]c3c2=O)c1] |
| 40 | 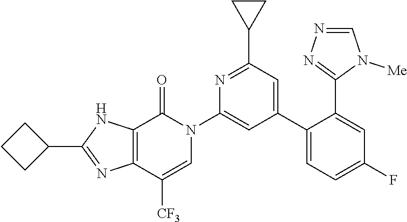 [SMILES: Cn1cnnc1-c1cc(F)ccc1-c1cc(C2CC2)nc(-n2cc(C(F)(F)F)c3nc(C4CCC4)[nH]c3c2=O)c1] |
| 41 | 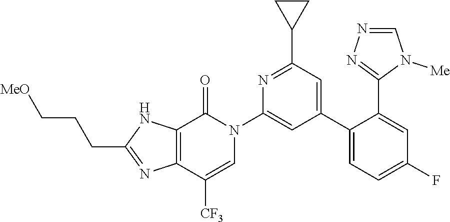 [SMILES: COCCCc1nc2c(C(F)(F)F)cn(-c3cc(-c4ccc(F)cc4-c4nncn4C)cc(C4CC4)n3)c(=O)c2[nH]1] |
| 42 | 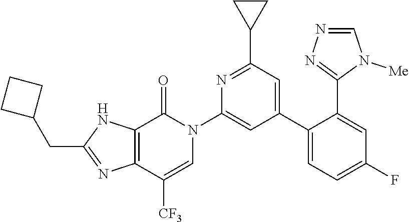 [SMILES: Cn1cnnc1-c1cc(F)ccc1-c1cc(C2CC2)nc(-n2cc(C(F)(F)F)c3nc(CC4CCC4)[nH]c3c2=O)c1] |
| 43 | 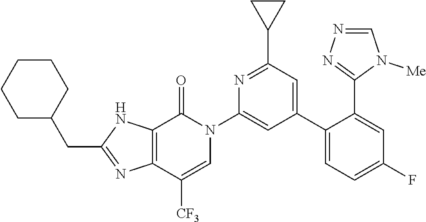 [SMILES: Cn1cnnc1-c1cc(F)ccc1-c1cc(C2CC2)nc(-n2cc(C(F)(F)F)c3nc(CC4CCCCC4)[nH]c3c2=O)c1] |
| 44 | 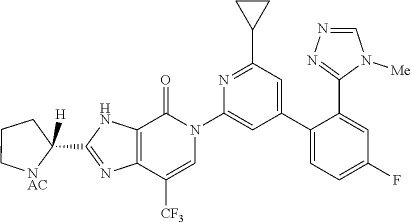 |
| 45 | 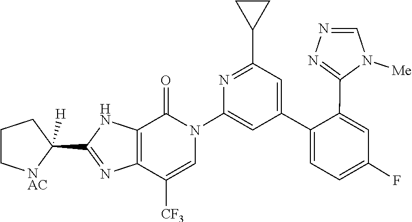 |
| 46 | 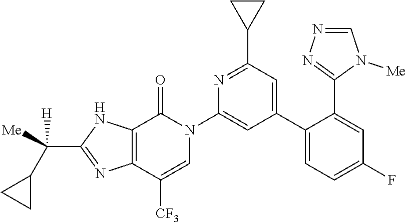 [SMILES: C[C@@H](c1nc2c(C(F)(F)F)cn(-c3cc(-c4ccc(F)cc4-c4nncn4C)cc(C4CC4)n3)c(=O)c2[nH]1)C1CC1] |
| 47 | 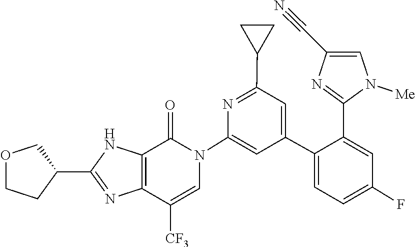 [SMILES: Cn1cc(C#N)nc1-c1cc(F)ccc1-c1cc(C2CC2)nc(-n2cc(C(F)(F)F)c3nc([C@@H]4CCOC4)[nH]c3c2=O)c1] |
| 48 | 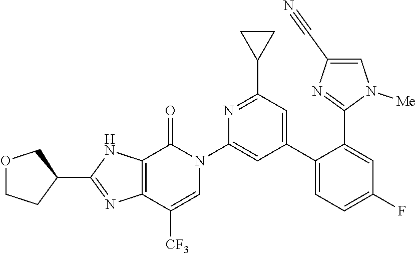 [SMILES: Cn1cc(C#N)nc1-c1cc(F)ccc1-c1cc(C2CC2)nc(-n2cc(C(F)(F)F)c3nc([C@H]4CCOC4)[nH]c3c2=O)c1] |
| 49 | 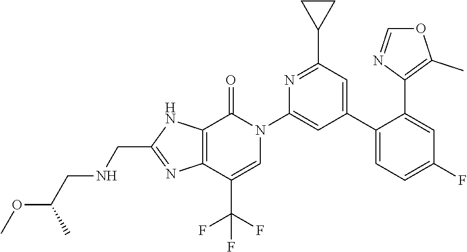 |
| 50 | 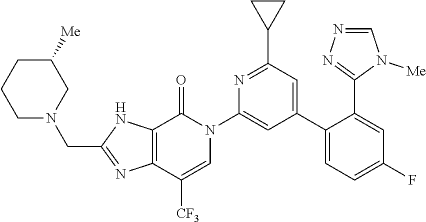 [SMILES: C[C@H]1CCCN(Cc2nc3c(C(F)(F)F)cn(-c4cc(-c5ccc(F)cc5-c5nncn5C)cc(C5CC5)n4)c(=O)c3[nH]2)C1] |
| 51 | 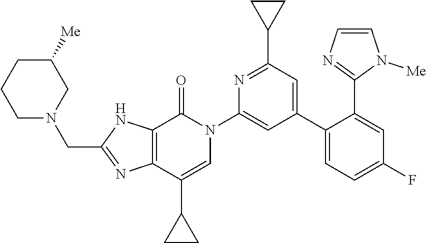 [SMILES: C[C@H]1CCCN(Cc2nc3c(C4CC4)cn(-c4cc(-c5ccc(F)cc5-c5nccn5C)cc(C5CC5)n4)c(=O)c3[nH]2)C1] |
| 52 | 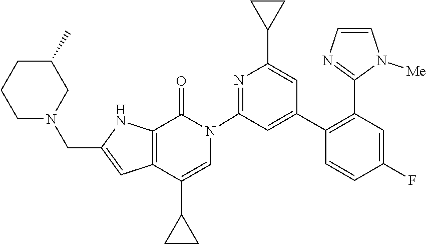 [SMILES: C[C@H]1CCCN(Cc2cc3c(C4CC4)cn(-c4cc(-c5ccc(F)cc5-c5nccn5C)cc(C5CC5)n4)c(=O)c3[nH]2)C1] |
| 53 | 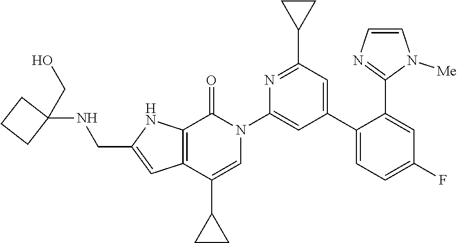 [SMILES: Cn1ccnc1-c1cc(F)ccc1-c1cc(C2CC2)nc(-n2cc(C3CC3)c3cc(CNC4(CO)CCC4)[nH]c3c2=O)c1] |
| 54 | 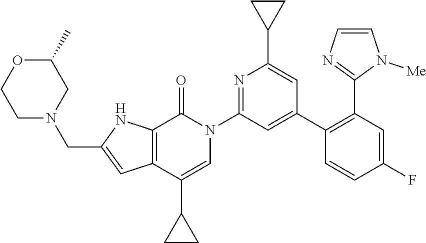 [SMILES: C[C@@H]1CN(Cc2cc3c(C4CC4)cn(-c4cc(-c5ccc(F)cc5-c5nccn5C)cc(C5CC5)n4)c(=O)c3[nH]2)CCO1] |
| 55 | 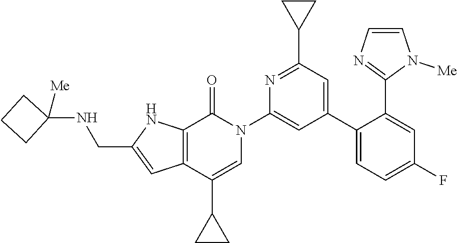 [SMILES: Cn1ccnc1-c1cc(F)ccc1-c1cc(C2CC2)nc(-n2cc(C3CC3)c3cc(CNC4(C)CCC4)[nH]c3c2=O)c1] |
| 56 | 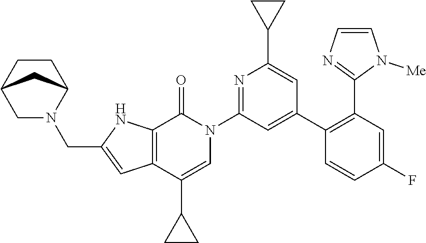 [SMILES: Cn1ccnc1-c1cc(F)ccc1-c1cc(C2CC2)nc(-n2cc(C3CC3)c3cc(CN4C[C@@H]5CC[C@H]4C5)[nH]c3c2=O)c1] |
| 57 | 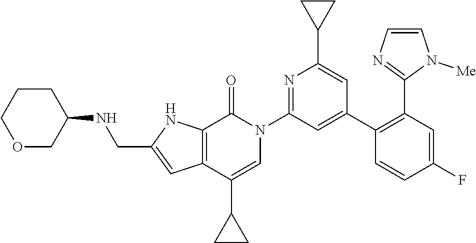 [SMILES: Cn1ccnc1-c1cc(F)ccc1-c1cc(C2CC2)nc(-n2cc(C3CC3)c3cc(CN[C@@H]4CCCOC4)[nH]c3c2=O)c1] |
| 58 | 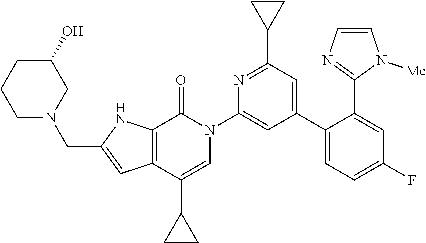 [SMILES: Cn1ccnc1-c1cc(F)ccc1-c1cc(C2CC2)nc(-n2cc(C3CC3)c3cc(CN4CCC[C@H](O)C4)[nH]c3c2=O)c1] |
| 59 | 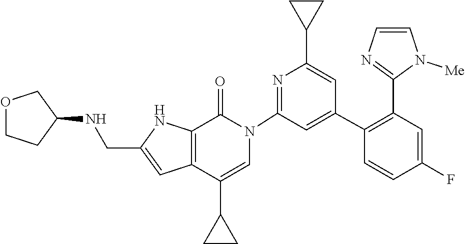 [SMILES: Cn1ccnc1-c1cc(F)ccc1-c1cc(C2CC2)nc(-n2cc(C3CC3)c3cc(CN[C@H]4CCOC4)[nH]c3c2=O)c1] |
| 60 | 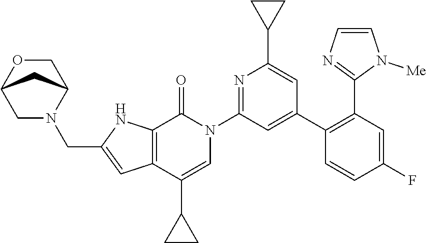 [SMILES: Cn1ccnc1-c1cc(F)ccc1-c1cc(C2CC2)nc(-n2cc(C3CC3)c3cc(CN4C[C@H]5C[C@@H]4CO5)[nH]c3c2=O)c1] |
| 61 | 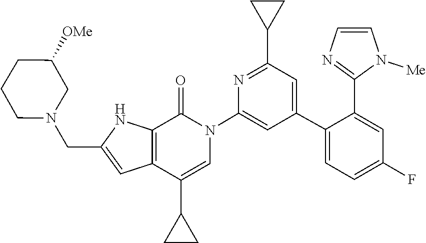 [SMILES: CO[C@H]1CCCN(Cc2cc3c(C4CC4)cn(-c4cc(-c5ccc(F)cc5-c5nccn5C)cc(C5CC5)n4)c(=O)c3[nH]2)C1] |
| 62 | 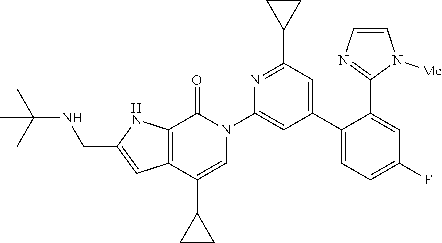 [SMILES: Cn1ccnc1-c1cc(F)ccc1-c1cc(C2CC2)nc(-n2cc(C3CC3)c3cc(CNC(C)(C)C)[nH]c3c2=O)c1] |
| 63 | 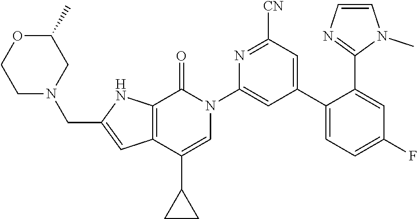 [SMILES: C[C@@H]1CN(Cc2cc3c(C4CC4)cn(-c4cc(-c5ccc(F)cc5-c5nccn5C)cc(C#N)n4)c(=O)c3[nH]2)CCO1] |
| 64 | 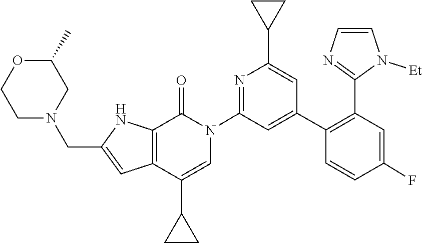 [SMILES: CCn1ccnc1-c1cc(F)ccc1-c1cc(C2CC2)nc(-n2cc(C3CC3)c3cc(CN4CCO[C@H](C)C4)[nH]c3c2=O)c1] |
| 65 | 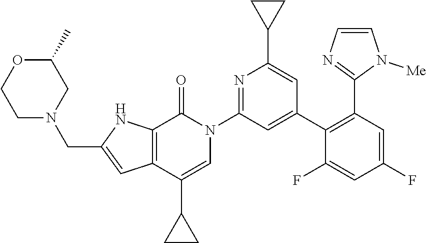 [SMILES: C[C@@H]1CN(Cc2cc3c(C4CC4)cn(-c4cc(-c5c(F)cc(F)cc5-c5nccn5C)cc(C5CC5)n4)c(=O)c3[nH]2)CCO1] |
| 66 | 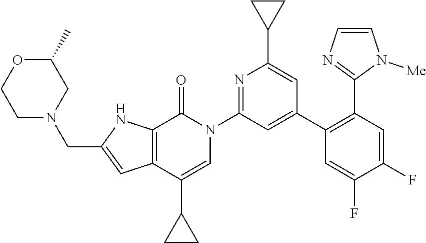 [SMILES: C[C@@H]1CN(Cc2cc3c(C4CC4)cn(-c4cc(-c5cc(F)c(F)cc5-c5nccn5C)cc(C5CC5)n4)c(=O)c3[nH]2)CCO1] |
| 67 | 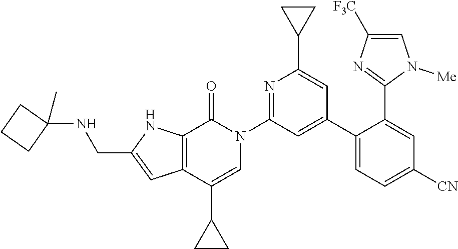 [SMILES: Cn1cc(C(F)(F)F)nc1-c1cc(C#N)ccc1-c1cc(C2CC2)nc(-n2cc(C3CC3)c3cc(CNC4(C)CCC4)[nH]c3c2=O)c1] |
| 68 | 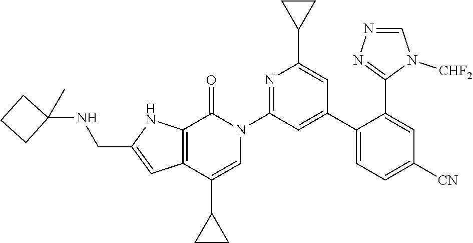 [SMILES: CC1(NCc2cc3c(C4CC4)cn(-c4cc(-c5ccc(C#N)cc5-c5nncn5C(F)F)cc(C5CC5)n4)c(=O)c3[nH]2)CCC1] |
| 69 | 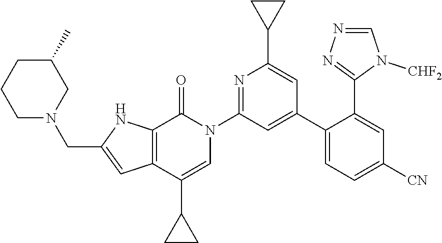 [SMILES: C[C@H]1CCCN(Cc2cc3c(C4CC4)cn(-c4cc(-c5ccc(C#N)cc5-c5nncn5C(F)F)cc(C5CC5)n4)c(=O)c3[nH]2)C1] |
| 70 | 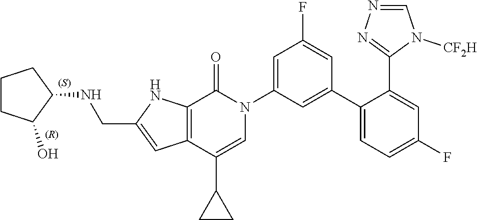 |
| 71 | 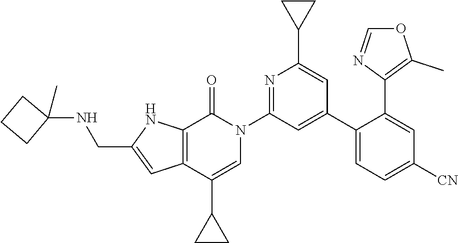 [SMILES: Cc1ocnc1-c1cc(C#N)ccc1-c1cc(C2CC2)nc(-n2cc(C3CC3)c3cc(CNC4(C)CCC4)[nH]c3c2=O)c1] |
| 72 | 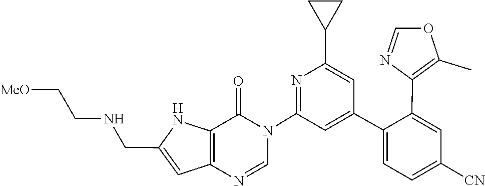 [SMILES: COCCNCc1cc2ncn(-c3cc(-c4ccc(C#N)cc4-c4ncoc4C)cc(C4CC4)n3)c(=O)c2[nH]1] |
| 73 | 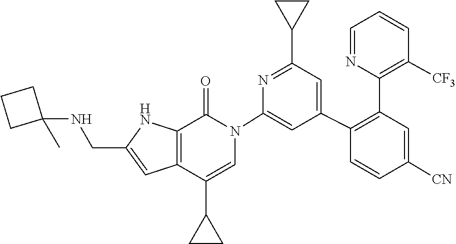 [SMILES: CC1(NCc2cc3c(C4CC4)cn(-c4cc(-c5ccc(C#N)cc5-c5ncccc5C(F)(F)F)cc(C5CC5)n4)c(=O)c3[nH]2)CCC1] |
| 74 | 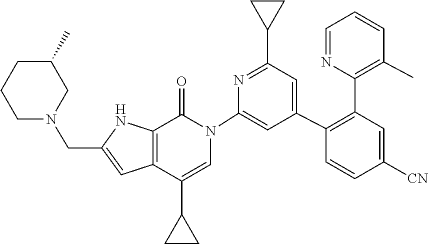 [SMILES: Cc1cccnc1-c1cc(C#N)ccc1-c1cc(C2CC2)nc(-n2cc(C3CC3)c3cc(CN4CCC[C@H](C)C4)[nH]c3c2=O)c1] |
| 75 | 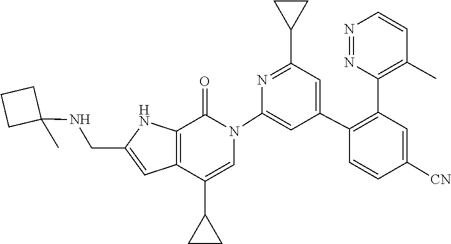 [SMILES: Cc1ccnnc1-c1cc(C#N)ccc1-c1cc(C2CC2)nc(-n2cc(C3CC3)c3cc(CNC4(C)CCC4)[nH]c3c2=O)c1] |
| 76 | 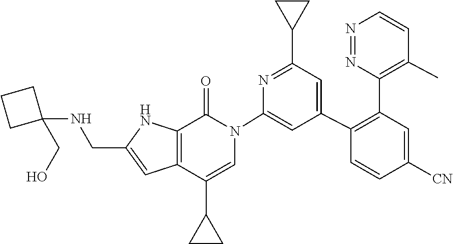 [SMILES: Cc1ccnnc1-c1cc(C#N)ccc1-c1cc(C2CC2)nc(-n2cc(C3CC3)c3cc(CNC4(CO)CCC4)[nH]c3c2=O)c1] |
| 77 | 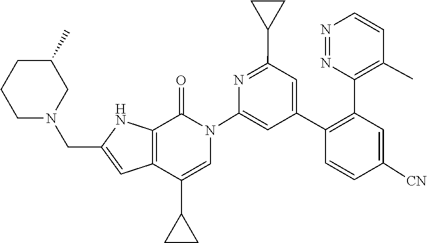 [SMILES: Cc1ccnnc1-c1cc(C#N)ccc1-c1cc(C2CC2)nc(-n2cc(C3CC3)c3cc(CN4CCC[C@H](C)C4)[nH]c3c2=O)c1] |
| 78 | 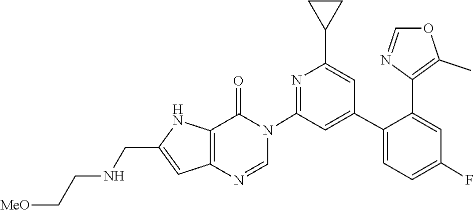 [SMILES: COCCNCc1cc2ncn(-c3cc(-c4ccc(F)cc4-c4ncoc4C)cc(C4CC4)n3)c(=O)c2[nH]1] |
| 79 | 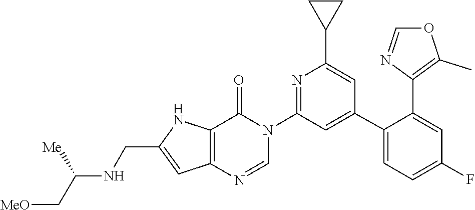 [SMILES: COC[C@H](C)NCc1cc2ncn(-c3cc(-c4ccc(F)cc4-c4ncoc4C)cc(C4CC4)n3)c(=O)c2[nH]1] |
| 80 | 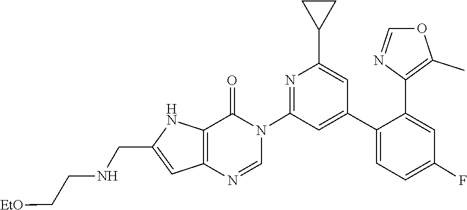 [SMILES: CCOCCNCc1cc2ncn(-c3cc(-c4ccc(F)cc4-c4ncoc4C)cc(C4CC4)n3)c(=O)c2[nH]1] |
| 81 | 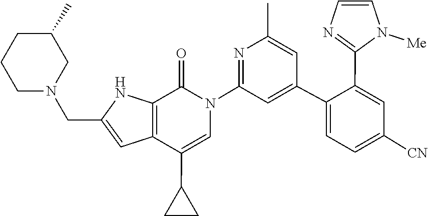 [SMILES: Cc1cc(-c2ccc(C#N)cc2-c2nccn2C)cc(-n2cc(C3CC3)c3cc(CN4CCC[C@H](C)C4)[nH]c3c2=O)n1] |
| 82 | 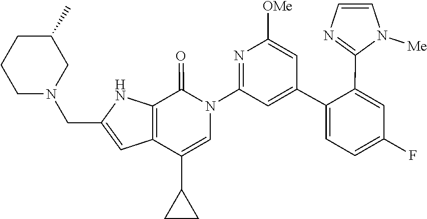 [SMILES: COc1cc(-c2ccc(F)cc2-c2nccn2C)cc(-n2cc(C3CC3)c3cc(CN4CCC[C@H](C)C4)[nH]c3c2=O)n1] |
| 83 | 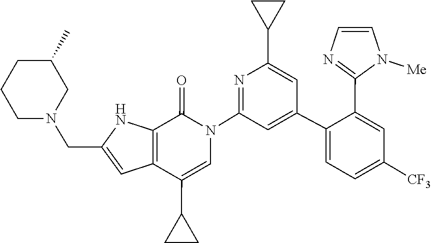 [SMILES: C[C@H]1CCCN(Cc2cc3c(C4CC4)cn(-c4cc(-c5ccc(C(F)(F)F)cc5-c5nccn5C)cc(C5CC5)n4)c(=O)c3[nH]2)C1] |
| 84 | 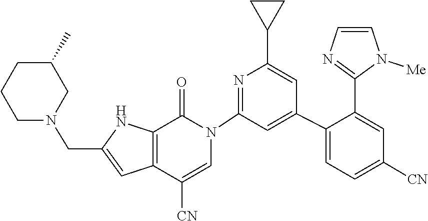 [SMILES: C[C@H]1CCCN(Cc2cc3c(C#N)cn(-c4cc(-c5ccc(C#N)cc5-c5nccn5C)cc(C5CC5)n4)c(=O)c3[nH]2)C1] |
| 85 | 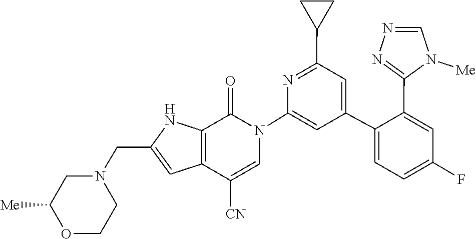 [SMILES: C[C@@H]1CN(Cc2cc3c(C#N)cn(-c4cc(-c5ccc(F)cc5-c5nncn5C)cc(C5CC5)n4)c(=O)c3[nH]2)CCO1] |
| 86 | 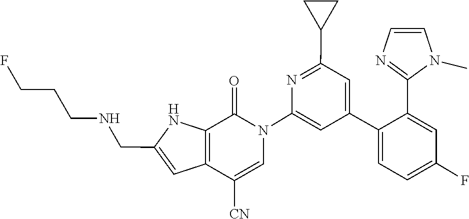 [SMILES: Cn1ccnc1-c1cc(F)ccc1-c1cc(C2CC2)nc(-n2cc(C#N)c3cc(CNCCCF)[nH]c3c2=O)c1] |
| 87 | 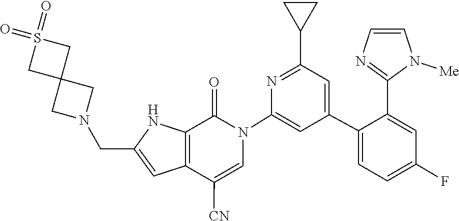 [SMILES: Cn1ccnc1-c1cc(F)ccc1-c1cc(C2CC2)nc(-n2cc(C#N)c3cc(CN4CC5(C4)CS(=O)(=O)C5)[nH]c3c2=O)c1] |
| 88 | 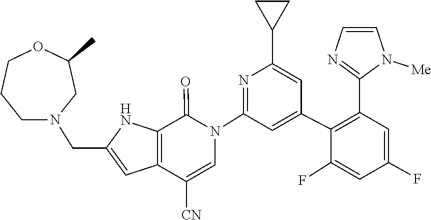 [SMILES: C[C@H]1CN(Cc2cc3c(C#N)cn(-c4cc(-c5c(F)cc(F)cc5-c5nccn5C)cc(C5CC5)n4)c(=O)c3[nH]2)CCCO1] |
| 89 | 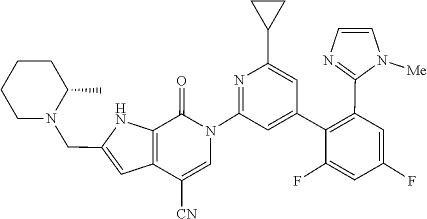 [SMILES: C[C@H]1CCCCN1Cc1cc2c(C#N)cn(-c3cc(-c4c(F)cc(F)cc4-c4nccn4C)cc(C4CC4)n3)c(=O)c2[nH]1] |
| 90 | 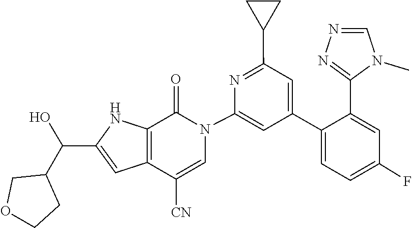 [SMILES: Cn1cnnc1-c1cc(F)ccc1-c1cc(C2CC2)nc(-n2cc(C#N)c3cc(C(O)C4CCOC4)[nH]c3c2=O)c1] |
| 91 | 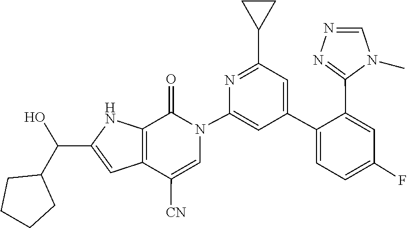 [SMILES: Cn1cnnc1-c1cc(F)ccc1-c1cc(C2CC2)nc(-n2cc(C#N)c3cc(C(O)C4CCCC4)[nH]c3c2=O)c1] |
| 92 | 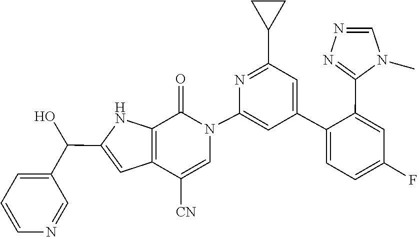 [SMILES: Cn1cnnc1-c1cc(F)ccc1-c1cc(C2CC2)nc(-n2cc(C#N)c3cc(C(O)c4cccnc4)[nH]c3c2=O)c1] |
| 93 | 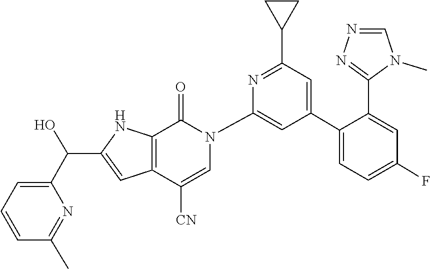 [SMILES: Cc1cccc(C(O)c2cc3c(C#N)cn(-c4cc(-c5ccc(F)cc5-c5nncn5C)cc(C5CC5)n4)c(=O)c3[nH]2)n1] |
| 94 | 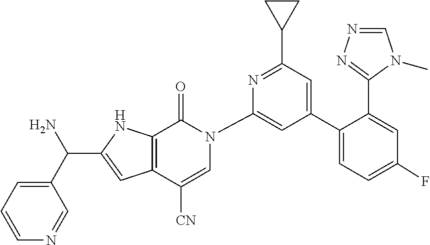 [SMILES: Cn1cnnc1-c1cc(F)ccc1-c1cc(C2CC2)nc(-n2cc(C#N)c3cc(C(N)c4cccnc4)[nH]c3c2=O)c1] |
| 95 | 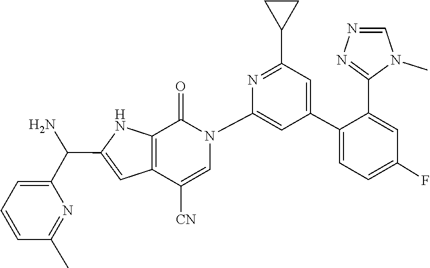 [SMILES: Cc1cccc(C(N)c2cc3c(C#N)cn(-c4cc(-c5ccc(F)cc5-c5nncn5C)cc(C5CC5)n4)c(=O)c3[nH]2)n1] |
| 96 | 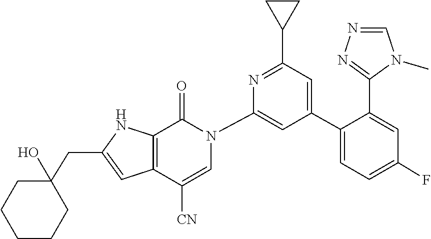 [SMILES: Cn1cnnc1-c1cc(F)ccc1-c1cc(C2CC2)nc(-n2cc(C#N)c3cc(CC4(O)CCCCC4)[nH]c3c2=O)c1] |
| 97 | 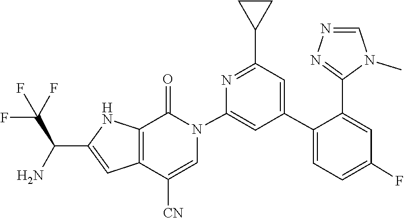 [SMILES: Cn1cnnc1-c1cc(F)ccc1-c1cc(C2CC2)nc(-n2cc(C#N)c3cc([C@@H](N)C(F)(F)F)[nH]c3c2=O)c1] |
| 98 | 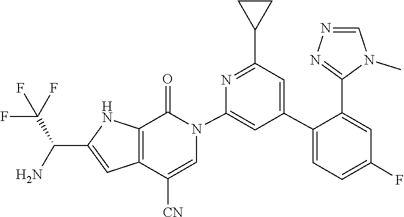 [SMILES: Cn1cnnc1-c1cc(F)ccc1-c1cc(C2CC2)nc(-n2cc(C#N)c3cc([C@H](N)C(F)(F)F)[nH]c3c2=O)c1] |
| 99 | 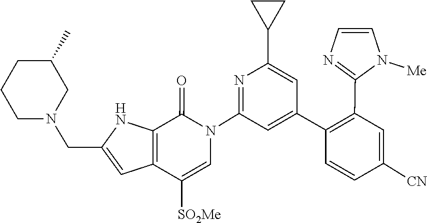 [SMILES: C[C@H]1CCCN(Cc2cc3c(S(C)(=O)=O)cn(-c4cc(-c5ccc(C#N)cc5-c5nccn5C)cc(C5CC5)n4)c(=O)c3[nH]2)C1] |
| 100 | 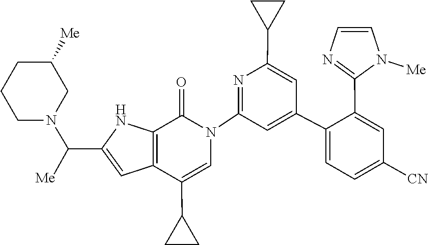 [SMILES: CC(c1cc2c(C3CC3)cn(-c3cc(-c4ccc(C#N)cc4-c4nccn4C)cc(C4CC4)n3)c(=O)c2[nH]1)N1CCC[C@H](C)C1] |
| 101 | 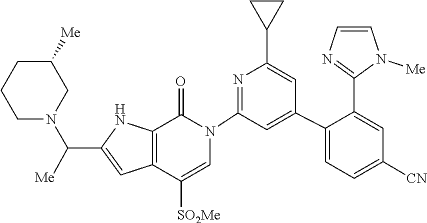 [SMILES: CC(c1cc2c(S(C)(=O)=O)cn(-c3cc(-c4ccc(C#N)cc4-c4nccn4C)cc(C4CC4)n3)c(=O)c2[nH]1)N1CCC[C@H](C)C1] |
| 102 | 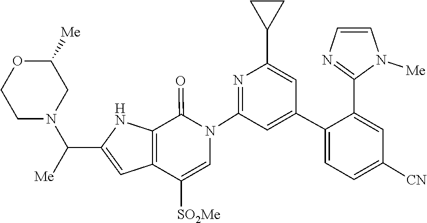 [SMILES: CC(c1cc2c(S(C)(=O)=O)cn(-c3cc(-c4ccc(C#N)cc4-c4nccn4C)cc(C4CC4)n3)c(=O)c2[nH]1)N1CCO[C@H](C)C1] |
| 103 | 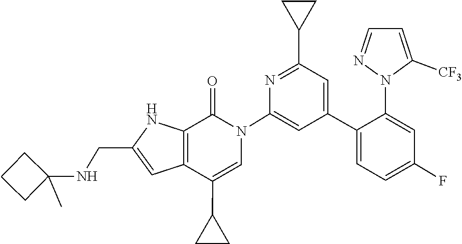 [SMILES: CC1(NCc2cc3c(C4CC4)cn(-c4cc(-c5ccc(F)cc5-n5nccc5C(F)(F)F)cc(C5CC5)n4)c(=O)c3[nH]2)CCC1] |
| 104 | 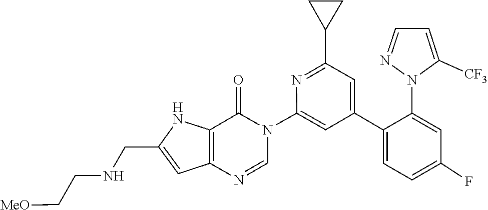 [SMILES: COCCNCc1cc2ncn(-c3cc(-c4ccc(F)cc4-n4nccc4C(F)(F)F)cc(C4CC4)n3)c(=O)c2[nH]1] |
| 105 | 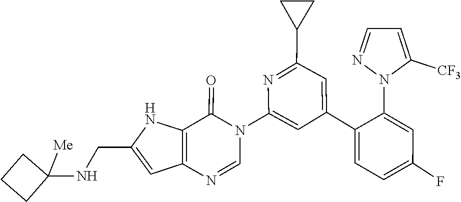 [SMILES: CC1(NCc2cc3ncn(-c4cc(-c5ccc(F)cc5-n5nccc5C(F)(F)F)cc(C5CC5)n4)c(=O)c3[nH]2)CCC1] |
| 106 | 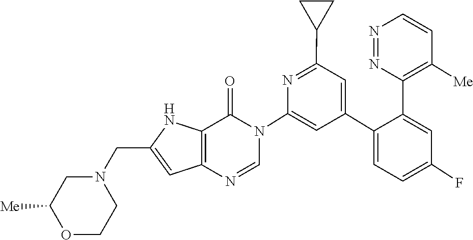 [SMILES: Cc1ccnnc1-c1cc(F)ccc1-c1cc(C2CC2)nc(-n2cnc3cc(CN4CCO[C@H](C)C4)[nH]c3c2=O)c1] |
| 107 | 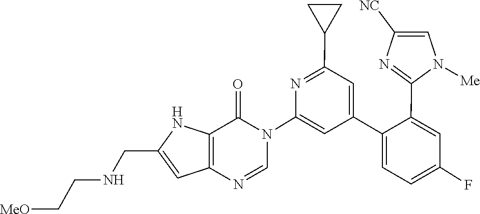 [SMILES: COCCNCc1cc2ncn(-c3cc(-c4ccc(F)cc4-c4nc(C#N)cn4C)cc(C4CC4)n3)c(=O)c2[nH]1] |
| 108 | 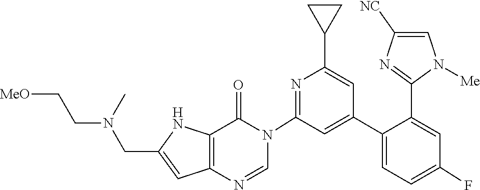 [SMILES: COCCN(C)Cc1cc2ncn(-c3cc(-c4ccc(F)cc4-c4nc(C#N)cn4C)cc(C4CC4)n3)c(=O)c2[nH]1] |
| 109 | 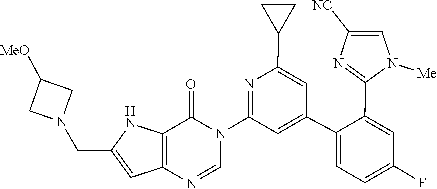 [SMILES: COC1CN(Cc2cc3ncn(-c4cc(-c5ccc(F)cc5-c5nc(C#N)cn5C)cc(C5CC5)n4)c(=O)c3[nH]2)C1] |
| 110 | 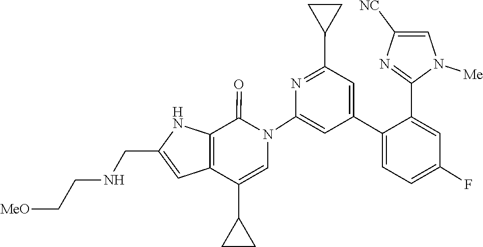 [SMILES: COCCNCc1cc2c(C3CC3)cn(-c3cc(-c4ccc(F)cc4-c4nc(C#N)cn4C)cc(C4CC4)n3)c(=O)c2[nH]1] |
| 111 | 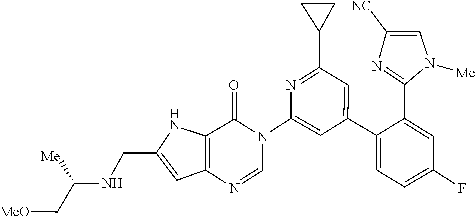 [SMILES: COC[C@H](C)NCc1cc2ncn(-c3cc(-c4ccc(F)cc4-c4nc(C#N)cn4C)cc(C4CC4)n3)c(=O)c2[nH]1] |
| 112 | 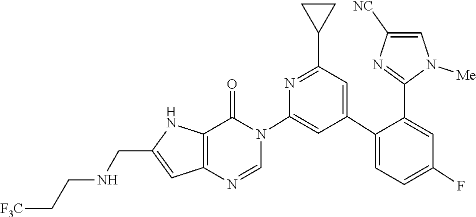 [SMILES: Cn1cc(C#N)nc1-c1cc(F)ccc1-c1cc(C2CC2)nc(-n2cnc3cc(CNCCC(F)(F)F)[nH]c3c2=O)c1] |
| 113 | 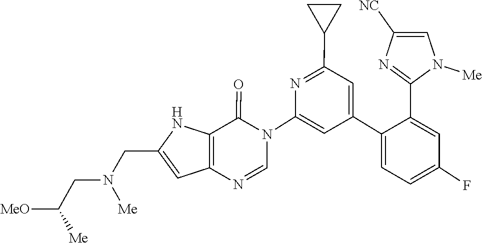 [SMILES: CO[C@@H](C)CN(C)Cc1cc2ncn(-c3cc(-c4ccc(F)cc4-c4nc(C#N)cn4C)cc(C4CC4)n3)c(=O)c2[nH]1] |
| 114-major | 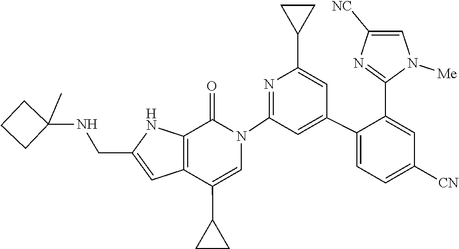 [SMILES: Cn1cc(C#N)nc1-c1cc(C#N)ccc1-c1cc(C2CC2)nc(-n2cc(C3CC3)c3cc(CNC4(C)CCC4)[nH]c3c2=O)c1] |
| 114-minor | 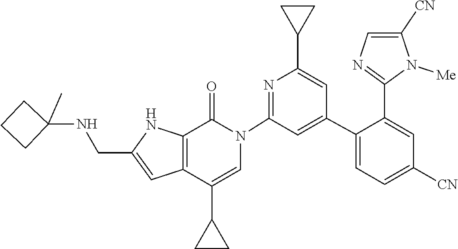 [SMILES: Cn1c(C#N)cnc1-c1cc(C#N)ccc1-c1cc(C2CC2)nc(-n2cc(C3CC3)c3cc(CNC4(C)CCC4)[nH]c3c2=O)c1] |
| 115 | 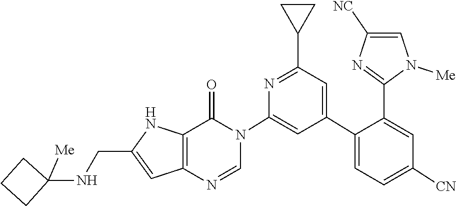 [SMILES: Cn1cc(C#N)nc1-c1cc(C#N)ccc1-c1cc(C2CC2)nc(-n2cnc3cc(CNC4(C)CCC4)[nH]c3c2=O)c1] |
| 116 | 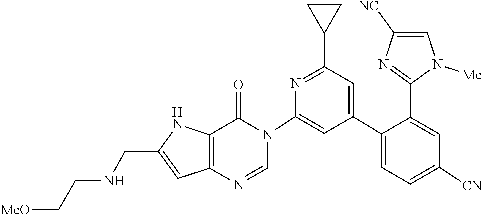 [SMILES: COCCNCc1cc2ncn(-c3cc(-c4ccc(C#N)cc4-c4nc(C#N)cn4C)cc(C4CC4)n3)c(=O)c2[nH]1] |
| 117 | 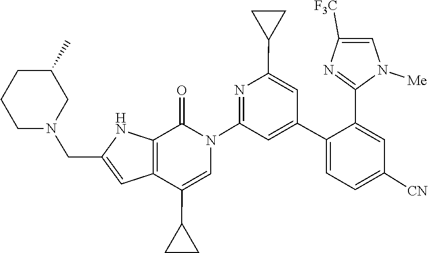 [SMILES: C[C@H]1CCCN(Cc2cc3c(C4CC4)cn(-c4cc(-c5ccc(C#N)cc5-c5nc(C(F)(F)F)cn5C)cc(C5CC5)n4)c(=O)c3[nH]2)C1] |
| 118 | 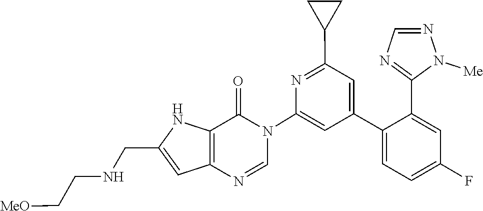 [SMILES: COCCNCc1cc2ncn(-c3cc(-c4ccc(F)cc4-c4ncnn4C)cc(C4CC4)n3)c(=O)c2[nH]1] |
| 119 | 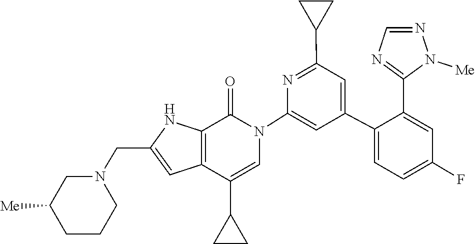 [SMILES: C[C@H]1CCCN(Cc2cc3c(C4CC4)cn(-c4cc(-c5ccc(F)cc5-c5ncnn5C)cc(C5CC5)n4)c(=O)c3[nH]2)C1] |
| 120 | 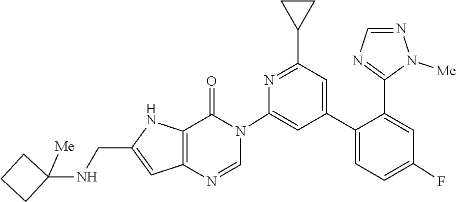 [SMILES: Cn1ncnc1-c1cc(F)ccc1-c1cc(C2CC2)nc(-n2cnc3cc(CNC4(C)CCC4)[nH]c3c2=O)c1] |
| 121 | 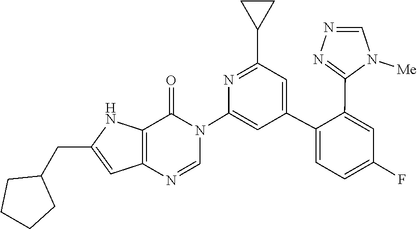 [SMILES: Cn1cnnc1-c1cc(F)ccc1-c1cc(C2CC2)nc(-n2cnc3cc(CC4CCCC4)[nH]c3c2=O)c1] |
| 122 | 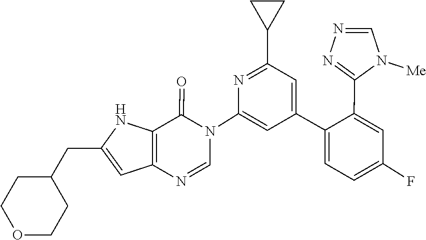 [SMILES: Cn1cnnc1-c1cc(F)ccc1-c1cc(C2CC2)nc(-n2cnc3cc(CC4CCOCC4)[nH]c3c2=O)c1] |
| 123 | 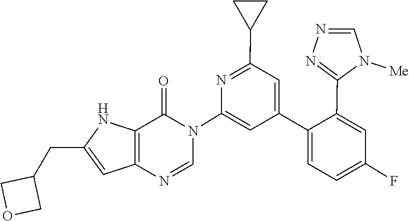 [SMILES: Cn1cnnc1-c1cc(F)ccc1-c1cc(C2CC2)nc(-n2cnc3cc(CC4COC4)[nH]c3c2=O)c1] |
| 124 | 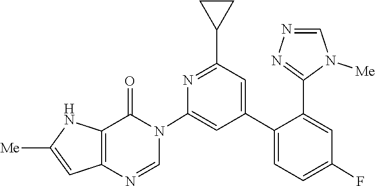 [SMILES: Cc1cc2ncn(-c3cc(-c4ccc(F)cc4-c4nncn4C)cc(C4CC4)n3)c(=O)c2[nH]1] |
| 125 | 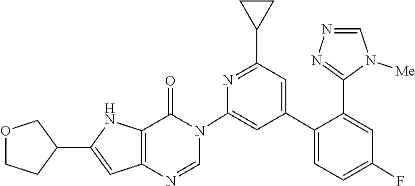 [SMILES: Cn1cnnc1-c1cc(F)ccc1-c1cc(C2CC2)nc(-n2cnc3cc(C4CCOC4)[nH]c3c2=O)c1] |
| 126 | 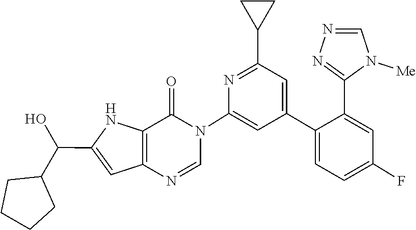 [SMILES: Cn1cnnc1-c1cc(F)ccc1-c1cc(C2CC2)nc(-n2cnc3cc(C(O)C4CCCC4)[nH]c3c2=O)c1] |
| 127 | 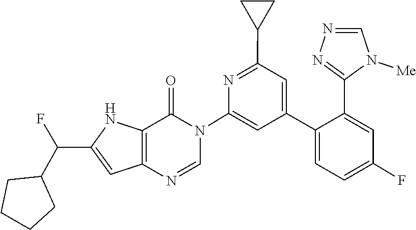 [SMILES: Cn1cnnc1-c1cc(F)ccc1-c1cc(C2CC2)nc(-n2cnc3cc(C(F)C4CCCC4)[nH]c3c2=O)c1] |
| 128 | 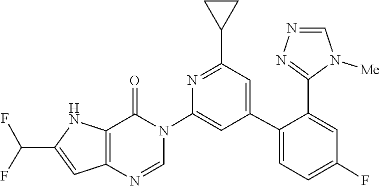 [SMILES: Cn1cnnc1-c1cc(F)ccc1-c1cc(C2CC2)nc(-n2cnc3cc(C(F)F)[nH]c3c2=O)c1] |
| 129 (Regio-1/ Regio-2) | 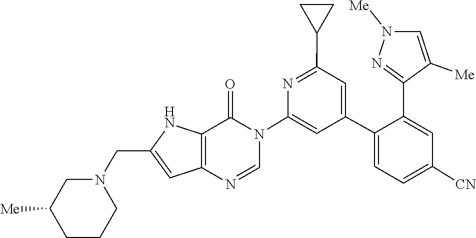 [SMILES: Cc1cn(C)nc1-c1cc(C#N)ccc1-c1cc(C2CC2)nc(-n2cnc3cc(CN4CCC[C@H](C)C4)[nH]c3c2=O)c1] |
| 129 (Regio-1/ Regio-2) | 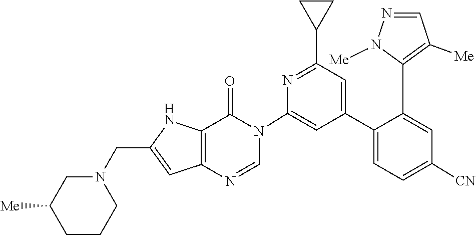 [SMILES: Cc1cnn(C)c1-c1cc(C#N)ccc1-c1cc(C2CC2)nc(-n2cnc3cc(CN4CCC[C@H](C)C4)[nH]c3c2=O)c1] |
| 130 | 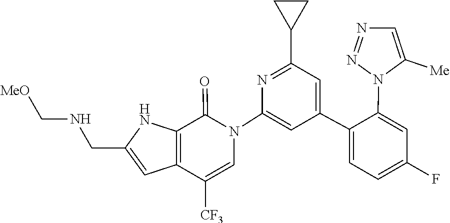 [SMILES: COCNCc1cc2c(C(F)(F)F)cn(-c3cc(-c4ccc(F)cc4-n4nncc4C)cc(C4CC4)n3)c(=O)c2[nH]1] |
| 131 | 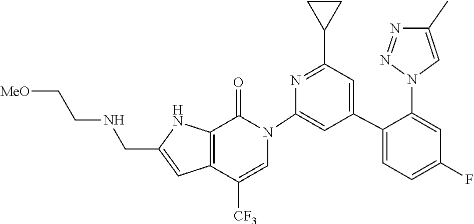 [SMILES: COCCNCc1cc2c(C(F)(F)F)cn(-c3cc(-c4ccc(F)cc4-n4cc(C)nn4)cc(C4CC4)n3)c(=O)c2[nH]1] |
| 132 | 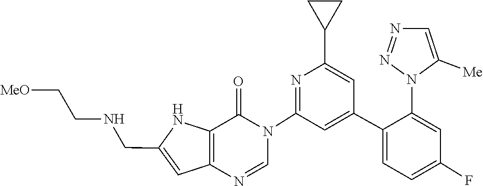 [SMILES: COCCNCc1cc2ncn(-c3cc(-c4ccc(F)cc4-n4nncc4C)cc(C4CC4)n3)c(=O)c2[nH]1] |
| 133 | 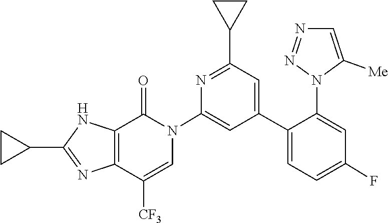 [SMILES: Cc1cnnn1-c1cc(F)ccc1-c1cc(C2CC2)nc(-n2cc(C(F)(F)F)c3nc(C4CC4)[nH]c3c2=O)c1] |
| 134 | 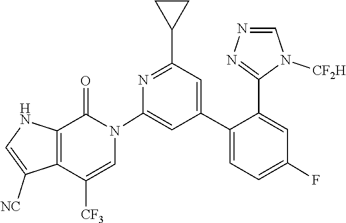 [SMILES: N#Cc1c[nH]c2c(=O)n(-c3cc(-c4ccc(F)cc4-c4nncn4C(F)F)cc(C4CC4)n3)cc(C(F)(F)F)c12] |
| 135 | 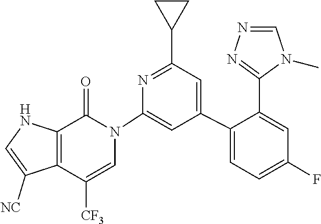 [SMILES: Cn1cnnc1-c1cc(F)ccc1-c1cc(C2CC2)nc(-n2cc(C(F)(F)F)c3c(C#N)c[nH]c3c2=O)c1] |
| 136 | 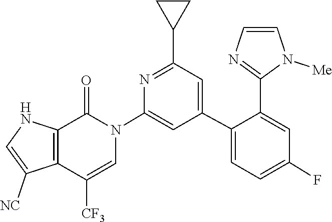 [SMILES: Cn1ccnc1-c1cc(F)ccc1-c1cc(C2CC2)nc(-n2cc(C(F)(F)F)c3c(C#N)c[nH]c3c2=O)c1] |
| 137 | 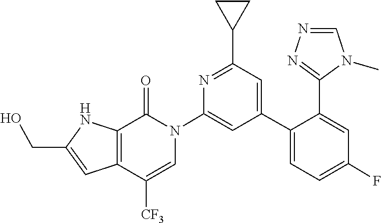 [SMILES: Cn1cnnc1-c1cc(F)ccc1-c1cc(C2CC2)nc(-n2cc(C(F)(F)F)c3cc(CO)[nH]c3c2=O)c1] |
| 138 | 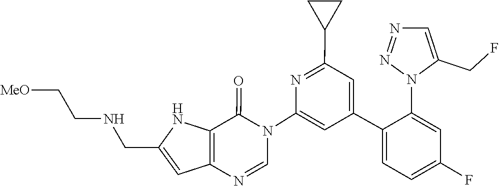 [SMILES: COCCNCc1cc2ncn(-c3cc(-c4ccc(F)cc4-n4nncc4CF)cc(C4CC4)n3)c(=O)c2[nH]1] |
| 139 | 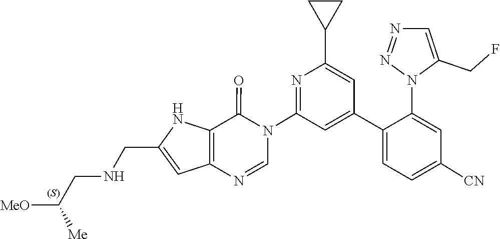 |
| 140 | 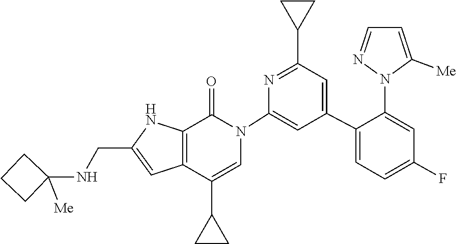 [SMILES: Cc1ccnn1-c1cc(F)ccc1-c1cc(C2CC2)nc(-n2cc(C3CC3)c3cc(CNC4(C)CCC4)[nH]c3c2=O)c1] |
| 141 | 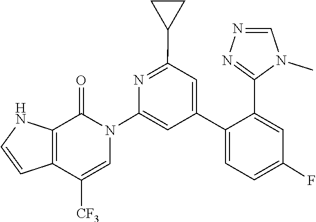 [SMILES: Cn1cnnc1-c1cc(F)ccc1-c1cc(C2CC2)nc(-n2cc(C(F)(F)F)c3cc[nH]c3c2=O)c1] |
| 142 | 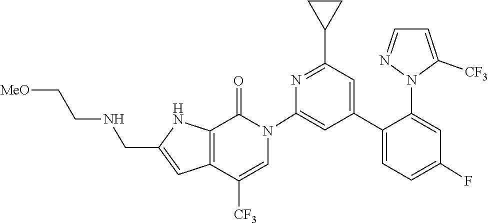 [SMILES: COCCNCc1cc2c(C(F)(F)F)cn(-c3cc(-c4ccc(F)cc4-n4nccc4C(F)(F)F)cc(C4CC4)n3)c(=O)c2[nH]1] |
| 143 | 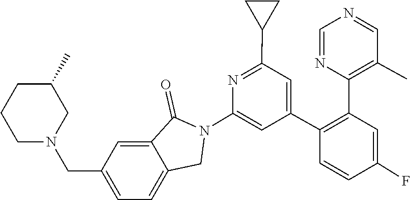 [SMILES: Cc1cncnc1-c1cc(F)ccc1-c1cc(C2CC2)nc(N2Cc3ccc(CN4CCC[C@H](C)C4)cc3C2=O)c1] |
| 144 | 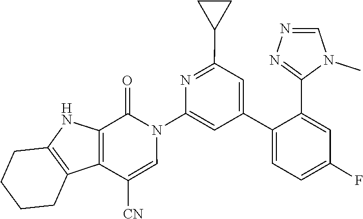 [SMILES: Cn1cnnc1-c1cc(F)ccc1-c1cc(C2CC2)nc(-n2cc(C#N)c3c4c([nH]c3c2=O)CCCC4)c1] |

### Therapeutic and Prophylactic Uses

The present disclosure provides methods for using compounds described herein in the preparation of a medicament for inhibiting Cbl-b. As used herein, the terms “inhibit”, ‘inhibition” and the like refer to the ability of a compound to decrease the function or activity of a particular target, e.g., Cbl-b. The decrease is preferably at least 5000 and may be, for example, at least about 5500, at least about 6000, at least about 65%, at least about 70%, at least about 75%, at least about 80%, at least about 85%, at least about 90%, or at least about 95%. The present disclosure also encompasses the use of the compounds described herein in the preparation of a medicament for the treatment or prevention of diseases, disorders, and/or conditions that would benefit from inhibition of Cbl-b. As one example, the present disclosure encompasses the use of the compounds described herein in the preparation of a medicament for the treatment of cancer. In another example, the present disclosure encompasses the use of the compounds described herein in the preparation of a medicament for the treatment of an infectious disease, optionally a viral infection. In some embodiments of the aforementioned methods, the compounds described herein are used in combination with at least one additional therapy, examples of which are set forth elsewhere herein.

Cbl-b is an E3 ubiquitin ligase that acts by ubiquitinating proteins leading to their degradation or altered subcellular localization. More specifically, Cbl-b acts by binding ubiquitin-conjugating enzyme (E2) loaded with ubiquitin and substrate to facilitate formation of an isopeptide bond between the C-terminal carboxyl of ubiquitin and the F-amino group of a substrate lysine side chain or free N-terminal amino group. Through this activity, Cbl-b functions, in one aspect, as a negative regulator of immune cell activation. For example, Cbl-b inhibits T cell activation through ubiquitination of intracellular signaling proteins, including but not limited to pTYR-containing proteins (e.g., ZAP-70, etc.), p85 regulatory subunit of phosphatidynlinositol 3 kinase (PI3K), PLCγ1, and PKCθ. Cbl-b is also believed to negatively regulate cytokine-induced or target-induced NK cell cytotoxicity and cytokine production. Cbl-b has also been implicated in immunosuppressive signaling pathways, such as PD-1, CTLA-4, CD155, and TGF-β.

As demonstrated herein, the use of compounds described herein potently inhibits Cbl-b activity, resulting in increased immune cell activity. Diseases, disorders, and/or conditions that would benefit from Cbl-b inhibition may include those where greater immune cell (e.g., T cell, NK cell, etc.) activation is desired and/or there is limited immune cell stimulation, for example, due to low antigen density, poor quality neoantigen, high PD-L1 expression, or combinations thereof.

Accordingly, in some embodiments, the compounds described herein are administered to a subject in need thereof in an amount effective to inhibit Cbl-b activity. In one example, a measure of Cbl-b inhibition may be decreased ubiquitination of intracellular signaling proteins targeted by Cbl-b. Non-limiting examples of intracellular signaling proteins targeted by Cbl-b include pTYR-containing proteins (e.g., ZAP-70, etc.), p85 regulatory subunit of phosphatidynlinositol 3 kinase (PI3K), PLCγ1, and PKCθ. Cbl-b activity may be assessed using primary immune cells (e.g., T cells, NK cells) obtained from a peripheral blood sample or a tissue sample (e.g., a tumor sample) that was obtained from the subject. Activity may be determined, for example, by comparison to a previous sample obtained from the subject (i.e., prior to administration of the compound) or by comparison to a reference value for a control group (e.g., standard of care, a placebo, etc.).

Alternatively or in addition, in some embodiments, the compounds described herein are administered to a subject in need thereof in an amount effective to increase immune cell expansion, proliferation, activation and/or activity, as compared to a suitable control (e.g., a subject receiving standard of care, a subject receiving no treatment or a placebo treatment, etc.). Immune cell expansion, proliferation, activation and activity may be assessed using cells obtained from a peripheral blood sample or a tissue sample (e.g., a tumor sample) that was obtained from the subject. Immune cell numbers in tissue or blood may be quantified (absolute numbers or relative numbers) by immunophenotyping, i.e., a process of using antibodies (or other antigen-specific reagent) to detect and quantify cell-associated antigens. Lymphoid cell markers may include but are not limited to CD3, CD4, CD8, CD16, CD25, CD39, CD45, CD56, CD103, CD127, and FOXP3. CD4 and CD8 can distinguish T cell with different effector functions (e.g., CD4+ T cells and CD8+ T cells). Co-expression of different cell markers can further distinguish sub-groups. For example, co-expression of CD39 and CD103 can differentiate tumor-specific T cells (CD8+CD39+CD103+ T cells) from bystander T cells in the tumor microenvironment (TME). For myeloid cells, suitable markers may include but are not limited to CD14, CD68, CD80, CD83, CD86, CD163, and CD206. Ki67 is a non-limiting example of a suitable marker of cell proliferation, such that an increase in Ki67 positive cells (e.g., CD8+ T cells, NK cells, etc.) as compared to a reference sample indicate cell proliferation. The term “activation” refers to the state of an immune cell that has been sufficiently primed to induce detectable effector functions (i.e., immune cell activity) upon stimulation. For example, T cells may be stimulated through the TCR/CD3 complex alone or with one or more secondary costimulatory signals. Non-limiting examples of measures of increased immune cell activity (i.e. effector function) may include increased expression, production and/or secretion of chemokines, pro-inflammatory cytokines and/or cytotoxic factors, increased cytotoxic activity, and increased gene expression and/or cell surface markers related to immune cell function and immune signaling. Examples of pro-inflammatory cytokines include, but are not limited to, IL-1a, IL-1b, IL-2, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of cytotoxic factors include, but are not limited to, granzyme A, granzyme B, soluble Fas ligand (sFasL), and perforin.

In some embodiments, the compounds described herein are administered to a subject in need thereof in an amount effective to increase T cell expansion, proliferation, activity, or any combination thereof. In certain embodiments, the T cells are CD8+ T cells, optionally tumor infiltrating CD8+ T cells and/or antigen experienced CD8+ T cells. In some embodiments, the T cells are CD8+CD39+CD103+ T cells. In embodiments directed to increased T cell activation and/or activity, measures of increased T cell activity may be increased T cell expression, production or secretion of chemokines, pro-inflammatory cytokines (e.g., IFNγ, TNF-α, IL-2, etc.) and/or cytotoxic factors (e.g. perforin, Granzyme B, etc.); increased pro-inflammatory cytokine levels in the tumor microenvironment; increased T cell receptor (TCR) signaling; increased glucose uptake; increased glycolysis; and increased killing of cancer cells. In some embodiments, the compounds described herein are administered to a subject in need thereof in an amount effective to increase activity, optionally wherein a measure of T cell activity is production and/or secretion of one or more pro-inflammatory cytokine, optionally wherein one or more pro-inflammatory cytokine is IFNγ, TNF-α, or IL-2.

In some embodiments, the compounds described herein are administered to a subject in need thereof in an amount effective to increase NK cell expansion, proliferation, activity, or any combination thereof. In embodiments directed to increased NK cell activity, measures of increased NK cell activity may be increased NK cell expression, production or secretion of chemokines, inflammatory cytokines (e.g., IFNγ, TNF-α, IL-2, etc.) and/or cytotoxic factors (e.g. perforin, Granzyme B, etc.); increased inflammatory cytokine levels in the tumor microenvironment; and increased killing of cancer cells.

Alternatively or in addition, in some embodiments, the compounds described herein are administered to a subject in need thereof to treat and/or prevent cancer or a cancer-related disease, disorder or condition. In some embodiments, the compounds described herein are administered to a subject in need thereof to treat cancer, optionally in combination with at least one additional therapy, examples of which are set forth elsewhere herein.

Alternatively or in addition, in some embodiments, the compounds described herein are administered to a subject in need thereof to treat and/or prevent an infection. In some embodiments, the compounds described herein are administered to a subject in need thereof to treat and/or prevent a viral infection. In some embodiments, the viral infection is a disease caused by hepatitis C virus (HCV), human papilloma virus (HPV), cytomegalovirus (CMV), herpes simplex virus (HSV), Epstein-Barr virus (EBV), varicella zoster virus, coxsackie virus, human immunodeficiency virus (HIV), or lymphocytic choriomeningitis virus (LCMV).

Alternatively or in addition, in some embodiments, the compounds described herein are brought into contact with an immune cell or a plurality of immune cells, in vitro or ex vivo, in an amount effective to increase proliferation, activation or activity of the immune cell(s). In some embodiments, the immune cell(s) may be allogenic immune cell(s) collected from one or more subjects. In some embodiments, the immune cell(s) may be autologous immune cell(s) collected from a subject in need of treatment. In certain embodiments, the cells may be “(re)programmed” allogenic immune cells produced from immune precursor cells (e.g., lymphoid progenitor cells, myeloid progenitor cells, common dendritic cell precursor cells, stem cells, induced pluripotent stem cells, etc.). In various embodiments, the immune cells may be genetically modified to target the cells to a specific antigen and/or enhance the cells' anti-tumor effects (e.g., engineered T cell receptor (TCR) cellular therapies, chimeric antigen receptor (CAR) cellular therapies, etc.). In some embodiments, the immune cell(s) are then administered to a subject in need thereof to treat and/or prevent cancer or a cancer-related disease, disorder or condition. In some embodiments, the immune cells are administered to a subject in need thereof to treat cancer, optionally in combination with at least one additional therapy, examples of which are set forth elsewhere herein.

In one or more embodiments, the compounds described herein are useful in the treatment and/or prophylaxis of cancer (e.g., carcinomas, sarcomas, leukemias, lymphomas, myelomas, etc.). In certain embodiments, the cancer may be locally advanced and/or unresectable, metastatic, or at risk of becoming metastatic. Alternatively, or in addition, the cancer may be recurrent or no longer responding to a treatment, such as a standard of care treatment known to one of skill in the art. In some embodiments, the cancer is resistant to treatment with immune checkpoint inhibitors (e.g., anti-PD-1 therapy), and/or chemotherapy (e.g., platinum-based chemotherapy). Exemplary types of cancer contemplated by this disclosure include cancer of the genitourinary tract (e.g., gynecologic, bladder, kidney, renal cell, penile, prostate, testicular, etc.), gastrointestinal tract (e.g., esophagus, oropharynx, stomach, small or large intestines, colon, or rectum), bone, bone marrow, skin (e.g., melanoma), head and neck, liver, gall bladder, bile ducts, heart, lung, pancreas, salivary gland, adrenal gland, thyroid, brain (e.g., gliomas), ganglia, central nervous system (CNS), peripheral nervous system (PNS), the hematopoietic system (i.e., hematological malignancies), the immune system (e.g., spleen or thymus), and cancers associated with Von Hippel-Lindau disease.

In some embodiments, the compounds according to this disclosure are useful in the treatment and/or prophylaxis of hematological malignancies. Exemplary types of cancer affecting the hematopoietic system include leukemias, lymphomas and myelomas, including acute myeloid leukemia, adult T-cell leukemia, T-cell large granular lymphocyte leukemia, acute lymphoblastic leukemia, chronic lymphocytic leukemia, chronic myelogenous leukemia, acute monocytic leukemia, Hodgkin's and Non-Hodgkin's lymphoma, Diffuse large B Cell lymphoma, and multiple myeloma. In a specific embodiment, the compounds according to this disclosure are useful in the treatment of Diffuse large B Cell lymphoma, optionally Diffuse large B Cell lymphoma with Richter transformation.

In another embodiment, the compounds according to this disclosure are useful in the treatment and/or prophylaxis of solid tumors. The solid tumor may be, for example, ovarian cancer, endometrial cancer, breast cancer, lung cancer (small cell or non-small cell), colon cancer, prostate cancer, cervical cancer, biliary cancer, pancreatic cancer, gastric cancer, esophageal cancer, liver cancer (hepatocellular carcinoma), kidney cancer (renal cell carcinoma), head-and-neck tumors, mesothelioma, melanoma, sarcomas, central nervous system (CNS) hemangioblastomas, and brain tumors (e.g., gliomas, such as astrocytoma, oligodendroglioma and glioblastomas).

In another embodiment, the compounds according to this disclosure are useful in the treatment and/or prophylaxis of breast cancer, genitourinary cancer, gastrointestinal cancer, lung cancer, skin cancer, or a combination thereof.

In some embodiments, the compounds according to this disclosure are useful in the treatment of breast cancer. In further embodiments, the breast cancer is hormone receptor positive (e.g., ERα-positive breast cancer, PR-positive breast cancer, ERα-positive and PR-positive breast cancer), HER2 positive breast cancer, HER2 over-expressing breast cancer, or any combination thereof. In still further embodiments, the breast cancer is triple negative breast cancer (TNBC).

In some embodiments, the compounds according to this disclosure are useful in the treatment of genitourinary cancer. In further embodiments, the genitourinary cancer is gynecologic cancer. In still further embodiments, the gynecologic cancer is cervical cancer, ovarian cancer (e.g., epithelial ovarian cancer (EOC)), vaginal cancer, vulvar cancer, endometrial cancer, peritoneal cancer, or fallopian tube carcinoma. In still further embodiments, the genitourinary cancer is urothelial cancer. In still further embodiments, the genitourinary cancer is prostate cancer, optionally castration-resistant prostate cancer. In further embodiments, the genitourinary cancer is bladder cancer. In still further embodiments, the genitourinary cancer is peritoneal cancer, optionally primary peritoneal cancer.

In some embodiments, the compounds according to this disclosure are useful in the treatment of head and neck cancer. In further embodiments, the head and neck cancer is head and neck squamous cell carcinoma (HNSCC).

In some embodiments, the compounds according to this disclosure are useful in the treatment of skin cancer. In further embodiments, the skin cancer is melanoma.

In some embodiments, the compounds according to this disclosure are useful in the treatment of lung cancer. In further embodiments, the lung cancer is mesothelioma or non-small cell lung cancer (NSCLC). In still further embodiments, the NSCLC is lung squamous cell carcinoma or lung adenocarcinoma. In further embodiments, the mesothelioma is malignant pleural mesothelioma (MPM).

In some embodiments, the compounds according to this disclosure are useful in the treatment of gastrointestinal cancer (GI). In some embodiments, the gastrointestinal cancer is upper GI cancer, such as esophageal or gastric cancer. In further embodiments, the upper GI cancer is an adenocarcinoma, a squamous cell carcinoma, or any combination thereof. In still further embodiments, the upper GI cancer is esophageal adenocarcinoma (EAC), esophageal squamous cell carcinoma (ESCC), gastroesophageal junction adenocarcinoma (GEJ), gastric adenocarcinoma (also referred to herein as “gastric cancer”) or any combination thereof. In some embodiments, the gastrointestinal cancer is lower GI cancer. In further embodiments, the lower GI cancer is colorectal cancer.

In some embodiments, the compounds according to this disclosure are useful in the treatment of a neuroendocrine tumor. In further embodiments, the neuroendocrine tumor is pancreatic neuroendocrine tumor, pheochromocytoma, paraganglioma, or a tumor of the adrenal gland.

In some embodiments, the compounds according to this disclosure are useful in the treatment of brain cancer. In further embodiments, the brain cancer is a glioma. In still further embodiments, the glioma is an astrocytoma, an oligodendroglioma, or a glioblastoma.

In some embodiments, the compounds according to this disclosure are useful in the treatment of kidney cancer. In further embodiments, the kidney cancer is renal cell carcinoma. In still further embodiments, the renal cell carcinoma is clear cell renal carcinoma.

In some embodiments, the compounds according to this disclosure are useful in the treatment of pancreatic cancer. In further embodiments, the pancreatic cancer is pancreatic neuroendocrine tumor or pancreatic adenocarcinoma.

In the aforementioned embodiments, the methods of the present disclosure may be practiced in an adjuvant setting or neoadjuvant setting, optionally in the treatment of locally advanced, unresectable, or metastatic cancer. Alternatively or in addition, the methods described herein may be indicated as a first line, second line, third line, or greater line of treatment, optionally in the treatment of locally advanced, unresectable, or metastatic cancer.

The present disclosure also provides methods of treating or preventing other cancer-related diseases, disorders or conditions. The use of the term(s) cancer-related diseases, disorders and conditions is meant to refer broadly to conditions that are associated, directly or indirectly, with cancer and non-cancerous proliferative disease, and includes, e.g., angiogenesis, precancerous conditions such as dysplasia, and non-cancerous proliferative diseases disorders or conditions, such as benign proliferative breast disease and papillomas. For clarity, the term(s) cancer-related disease, disorder and condition do not include cancer per se.

In general, the disclosed methods for treating or preventing cancer, or a cancer-related disease, disorder or condition, in a subject in need thereof comprise administering to the subject a compound disclosed herein, or a pharmaceutically acceptable salt thereof. In some embodiments, the present disclosure provides methods for treating or preventing cancer, or a cancer-related disease, disorder or condition with a compound disclosed herein, or a pharmaceutically acceptable salt thereof, and at least one additional therapy, examples of which are set forth elsewhere herein.

In particular embodiments of the present disclosure, the compounds are used to increase or enhance an immune response to an antigen by providing adjuvant activity. In a particular embodiment, at least one antigen or vaccine is administered to a subject in combination with at least one compound of the present disclosure to prolong an immune response to the antigen or vaccine. Therapeutic compositions are also provided which include at least one antigenic agent or vaccine component, including, but not limited to, viruses, bacteria, and fungi, or portions thereof, proteins, peptides, tumor-specific antigens, and nucleic acid vaccines, in combination with at least one compound of the present disclosure.

In some instances, the methods according to this disclosure may be provided in selected patients, for example subjects identified as having in a relevant tissue or sample, e.g., detectable PD-L1 expression, high microsatellite instability, high tumor mutational burden, or any combination thereof. In some instances, the subject is identified as having an oncogene driven cancer that has a mutation in at least one gene associated with the cancer.

In some embodiments, patients are selected by assessing the expression of relevant biomarkers, e.g., PD-L1 expression, microsatellite instability markers, etc., in a relevant sample, such as a peripheral blood sample or a tumor biopsy, using immunohistochemistry, immunophenotyping, PCR-based amplification, RNA sequencing, or other clinically validated assay. In one embodiment, the disclosure provides a method of treating cancer in a patient having (i) detectable PD-L1 expression, (ii) elevated PD-L1 expression, (iii) variability in the size of one, two, or more microsatellite repeats compared to normal cells, or (iv) any combination of (i) to (iii) by administering a compound as described herein. In another embodiment, the disclosure provides a method of treating cancer in a patient having (i) detectable PD-L1 expression, (ii) elevated PD-L1 expression, (iii) variability in the size of one, two, or more microsatellite repeats compared to normal cells, or (iv) any combination of (i) to (iii) by administering a therapeutically effective amount of a compound as described herein. In still another embodiment, the disclosure provides a method of administering a therapeutically effective amount of a compound as described herein to an individual for the treatment of cancer based on a determination of the relative amount of PD-L1 expression. In yet another embodiment, the disclosure provides a method of administering a therapeutically effective amount of a compound described herein to an individual for the treatment of cancer, the method comprising measuring PD-L1 expression and/or microsatellite instability in a sample obtained from an individual, for example by immunohistochemistry, immunophenotyping, PCR-based amplification, or other clinically validated test, and administering a therapeutically effective amount of the compound to the individual whose sample contained detectable PD-L1 expression.

### Routes of Administration

In some embodiments, pharmaceutical compositions containing a compound according to this disclosure may be in a form suitable for oral administration. Oral administration may involve swallowing the formulation thereby allowing the compound to be absorbed into the bloodstream in the gastrointestinal tract. Alternatively, oral administration may involve buccal, lingual or sublingual administration, thereby allowing the compound to be absorbed into the blood stream through oral mucosa.

In another embodiment, the pharmaceutical compositions containing a compound according to this disclosure may be in a form suitable for parenteral administration. Forms of parenteral administration include, but are not limited to, intravenous, intraarterial, intramuscular, intradermal, intraperitoneal, intrathecal, intracisternal, intracerebral, intracerebroventricular, intraventricular, and subcutaneous. Pharmaceutical compositions suitable for parenteral administration may be formulated using suitable aqueous or non-aqueous carriers. Depot injections, which are generally administered subcutaneously or intramuscularly, may also be utilized to release the compounds disclosed herein over a defined period of time.

Other routes of administration are also contemplated by this disclosure, including, but not limited to, nasal, vaginal, intraocular, rectal, topical (e.g., transdermal), and inhalation.

Particular embodiments of the present disclosure contemplate oral administration or parenteral administration.

### Pharmaceutical Compositions

The compounds of the present disclosure may be in the form of compositions suitable for administration to a subject. In general, such compositions are pharmaceutical compositions comprising a compound according to this disclosure or a pharmaceutically acceptable salt thereof and one or more pharmaceutically acceptable excipients. In some embodiments, the pharmaceutical composition comprises a compound according to this disclosure and one or more pharmaceutically acceptable excipients. In certain embodiments, the compound may be present in an effective amount. The pharmaceutical compositions may be used in the methods of the present disclosure; thus, for example, the pharmaceutical compositions comprising a compound according to this disclosure can be administered to a subject in order to practice the therapeutic and prophylactic methods and uses described herein.

The pharmaceutical compositions of the present disclosure can be formulated to be compatible with the intended method or route of administration. Routes of administration may include those known in the art. Exemplary routes of administration are oral and parenteral. Furthermore, the pharmaceutical compositions may be used in combination with one or more other therapies described herein in order to treat or prevent the diseases, disorders and conditions as contemplated by the present disclosure. In one embodiment, one or more other therapeutic agents contemplated by this disclosure are included in the same pharmaceutical composition that comprises the compound according to this disclosure. In another embodiment, the one or more other therapeutical agents are in a composition that is separate from the pharmaceutical composition comprising the compound according to this disclosure.

In one aspect, the compounds described herein may be administered orally. Oral administration may be via, for example, capsule or tablets. In making the pharmaceutical compositions that include the compounds of the present disclosure (e.g., a of Formula I), or a pharmaceutically acceptable salt thereof, the tablet or capsule includes at least one pharmaceutically acceptable excipient. Non-limiting examples of pharmaceutically acceptable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, sterile water, syrup, and methyl cellulose. Additional pharmaceutically acceptable excipients include lubricating agents such as talc, magnesium stearate, and mineral oil; wetting agents; emulsifying and suspending agents; preserving agents such as methyl and propylhydroxy-benzoates.

In another aspect, the compounds of the present disclosure, or a pharmaceutically acceptable salt thereof, may be administered parenterally, for example by intravenous injection. A pharmaceutical composition appropriate for parenteral administration may be formulated in solution for injection or may be reconstituted for injection in an appropriate system such as a physiological solution. Such solutions may include sterile water for injection, salts, buffers, and tonicity excipients in amounts appropriate to achieve isotonicity with the appropriate physiology.

The pharmaceutical compositions described herein may be stored in an appropriate sterile container or containers. In some embodiments, the container is designed to maintain stability for the pharmaceutical composition over a given period of time.

### Administering

In general, the disclosed methods comprise administering a compound described herein, or a composition thereof, in an effective amount to a subject in need thereof. An “effective amount” with reference to a Cbl-b inhibitor of the present disclosure means an amount of the compound that is sufficient to engage the target (e.g., by inhibiting the target) at a level that is indicative of the potency of the compound. For Cbl-b, target engagement can be determined by one or more biochemical or cellular assays resulting in an EC50, ED50, EC90, IC50, or similar value which can be used as one assessment of the potency of the compound. Assays for determining target engagement include, but are not limited to, those described in the Examples. The effective amount may be administered as a single quantity or as multiple, smaller quantities (e.g., as one tablet with “x” amount, as two tablets each with “x/2” amount, etc.).

In some embodiments, the disclosed methods comprise administering a therapeutically effective amount of a compound described herein to a subject in need thereof. As used herein, the phrase “therapeutically effective amount” with reference to compound disclosed herein means a dose regimen (i.e., amount and interval) of the compound that provides the specific pharmacological effect for which the compound is administered to a subject in need of such treatment. For prophylactic use, a therapeutically effective amount may be effective to eliminate or reduce the risk, lessen the severity, or delay the onset of the disease, including biochemical, histological and/or behavioral signs or symptoms of the disease. For treatment, a therapeutically effective amount may be effective to reduce, ameliorate, or eliminate one or more signs or symptoms associated with a disease, delay disease progression, prolong survival, decrease the dose of other medication(s) required to treat the disease, or a combination thereof. With respect to cancer specifically, a therapeutically effective amount may, for example, result in the killing of cancer cells, reduce cancer cell counts, reduce tumor burden, eliminate tumors or metastasis, or reduce metastatic spread. A therapeutically effective amount may vary based on, for example, one or more of the following: the age and weight of the subject, the subject's overall health, the stage of the subject's disease, the route of administration, and prior or concomitant treatments.

Administration may comprise one or more (e.g., one, two, or three or more) dosing cycles.

In certain embodiments, the compounds contemplated by the present disclosure may be administered (e.g., orally, parenterally, etc.) at about 0.01 mg/kg to about 50 mg/kg, or about 1 mg/kg to about 25 mg/kg, of subject's body weight per day, one or more times a day, a week, or a month, to obtain the desired effect. In some embodiments, once daily or twice daily administration is contemplated. In some embodiments, a suitable weight-based dose of a compound contemplated by the present disclosure is used to determine a dose that is administered independent of a subject's body weight. In certain embodiments, the compounds of the present disclosure are administered (e.g., orally, parenterally, etc.) at fixed dosage levels of about 1 mg to about 1000 mg, particularly 1, 3, 5, 10, 15, 20, 25, 50, 75, 100, 150, 200, 250, 300, 400, 500, 600, 750, 800, 900, or 1000 mg, one or more times a day, a week, or a month, to obtain the desired effect.

In certain embodiments, the compound is contained in a “unit dosage form”. The phrase “unit dosage form” refers to physically discrete units, each unit containing a predetermined amount of the compound, either alone or in combination with one or more additional agents, sufficient to produce the desired effect. It will be appreciated that the parameters of a unit dosage form will depend on the particular agent and the effect to be achieved.

### Combination Therapy

The present disclosure contemplates the use of compounds disclosed herein alone or in combination with one or more additional therapy. Each additional therapy can be a therapeutic agent or another treatment modality. In embodiments comprising one or more additional therapeutic agents, each agent may target a different, but complementary, mechanism of action. The additional therapeutic agents can be small chemical molecules; macromolecules such as proteins, antibodies, peptibodies, peptides, DNA, RNA or fragments of such macromolecules; or cellular or gene therapies. Non-limiting examples of additional treatment modalities include surgical resection of a tumor, bone marrow transplant, radiation therapy, and photodynamic therapy. The use of a compound disclosed herein in combination with one or more additional therapies may have a synergistic therapeutic or prophylactic effect on the underlying disease, disorder, or condition. In addition to or alternatively, the combination therapy may allow for a dose reduction of one or more of the therapies, thereby ameliorating, reducing or eliminating adverse effects associated with one or more of the agents.

In embodiments comprising one or more additional treatment modality, the compound can be administered before, after or during treatment with the additional treatment modality. In embodiments comprising one or more additional therapeutic agents, the therapeutic agents used in such combination therapy can be formulated as a single composition or as separate compositions. If administered separately, each therapeutic agent in the combination can be given at or around the same time, or at different times. Furthermore, the therapeutic agents are administered “in combination” even if they have different forms of administration (e.g., oral capsule and intravenous), they are given at different dosing intervals, one therapeutic agent is given at a constant dosing regimen while another is titrated up, titrated down or discontinued, or each therapeutic agent in the combination is independently titrated up, titrated down, increased or decreased in dosage, or discontinued and/or resumed during a subject's course of therapy. If the combination is formulated as separate compositions, in some embodiments, the separate compositions are provided together in a kit.

### Cancer Therapies

The present disclosure contemplates the use of the compounds described herein in combination with one or more additional therapies useful in the treatment of cancer.

In some embodiments, one or more of the additional therapies is an additional treatment modality. Exemplary treatment modalities include but are not limited to surgical resection of a tumor, bone marrow transplant, radiation therapy, and photodynamic therapy.

In some embodiments, one or more of the additional therapies is a therapeutic agent. Exemplary therapeutic agents include chemotherapeutic agents, radiopharmaceuticals, hormone therapies, epigenetic modulators, ATP-adenosine axis-targeting agents, targeted therapies, signal transduction inhibitors, RAS signaling inhibitors, PI3K inhibitors, arginase inhibitors, HIF inhibitors, AXL inhibitors, PAK4 inhibitors, immunotherapeutic agents, cellular therapies, gene therapies, immune checkpoint inhibitors, and agonists of stimulatory or co-stimulatory immune checkpoints.

In some embodiments, one or more of the additional therapeutic agents is a chemotherapeutic agent. Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, pomalidomide, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pemetrexed, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel, nab paclitaxel, and docetaxel; chlorambucil; gemcitabine; 6-thioguanine; mercaptopurine; methotrexate; platinum and platinum coordination complexes such as cisplatin, carboplatin and oxaliplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; proteasome inhibitors such as bortezomib, carfilzomib and ixazomib; topoisomerase inhibitors such as irinotecan, topotecan, etoposide, mitoxantrone, teniposide; difluoromethylornithine (DMFO); retinoic acid; esperamicins; capecitabine; anthracyclines and pharmaceutically acceptable salts, acids or derivatives of any of the above. In certain embodiments, combination therapy comprises a chemotherapy regimen that includes one or more chemotherapeutic agents. In one embodiment, combination therapy comprises a chemotherapeutic regimen comprising one or more of FOLFOX (folinic acid, fluorouracil, and oxaliplatin), FOLFIRI (e.g., folinic acid, fluorouracil, and irinotecan), FOLFIRINOX (folinic acid, fluorouracil, irinotecan, and oxaliplatin), CAPOX (capecitabine and oxaliplatin), a taxoid (e.g., docetaxel, paclitaxel, nab-paclitaxel, etc.), a fluoropyrimidine-containing chemotherapeutic agent (e.g., fluorouracil, capecitabine, floxuridine), a platinum-containing chemotherapeutic agent, and/or gemcitabine.

In some embodiments, one or more of the additional therapeutic agents is a radiopharmaceutical. A radiopharmaceutical is a form of internal radiation therapy in which a source of radiation (i.e., one or more radionuclide) is put inside a subject's body. The radiation source can be in solid or liquid form. Non-limiting examples of radiopharmaceuticals include sodium iodide I-131, radium-223 dichloride, lobenguane iodine-131, radioiodinated vesicles (e.g., saposin C-dioleoylphosphatidylserine (SapC-DOPS) nanovesicles), various forms of brachytherapy, and various forms of targeted radionuclides. Targeted radionuclides comprise a radionuclide associated (e.g., by covalent or ionic interactions) with a molecule (“a targeting agent”) that specifically binds to a target on a cell, typically a cancer cell or an immune cell. The targeting agent may be a small molecule, a saccharide (inclusive of oligosaccharides and polysaccharides), an antibody, a lipid, a protein, a peptide, a non-natural polymer, or an aptamer. In some embodiments, the targeting agent is a saccharide (inclusive of oligosaccharides and polysaccharides), a lipid, a protein, or a peptide and the target is a tumor-associated antigen (enriched but not specific to a cancer cell), a tumor-specific antigen (minimal to no expression in normal tissue), or a neo-antigen (an antigen specific to the genome of a cancer cell generated by non-synonymous mutations in the tumor cell genome). In some embodiments, the targeting agent is an antibody and the target is a tumor-associated antigen (i.e., an antigen enriched but not specific to a cancer cell), a tumor-specific antigen (i.e., an antigen with minimal to no expression in normal tissue), or a neo-antigen (i.e., an antigen specific to the genome of a cancer cell generated by non-synonymous mutations in the tumor cell genome). Non-limiting examples of targeted radionuclides include radionuclides attached to: somatostatin or peptide analogs thereof (e.g., 177Lu-Dotatate, etc.); prostate specific membrane antigen or peptide analogs thereof (e.g., 177Lu-PSMA-617, 225Ac-PSMA-617, 177Lu-PSMA-I&T, 177Lu-MIP-1095, etc.); a receptor's cognate ligand, peptide derived from the ligand, or variants thereof (e.g., 188 Re-labeled VEGF₁₂₅₋₁₃₆ or variants thereof with higher affinity to VEGF receptor, etc.); antibodies targeting tumor antigens (e.g., 131I-tositumomab, 90Y-ibritumomab tiuxetan, CAM-H2-I131 (Precirix NV), I131-omburtamab, etc.).

In some embodiments, one or more of the additional therapeutic agents is a hormone therapy. Hormone therapies act to regulate or inhibit hormonal action on tumors. Examples of hormone therapies include, but are not limited to: selective estrogen receptor degraders such as fulvestrant, giredestrant, SAR439859, RG6171, AZD9833, rintodestrant, ZN-c5, LSZ102, D-0502, LY3484356, SHR9549; selective estrogen receptor modulators such as tamoxifen, raloxifene, 4-hydroxytamoxifen, trioxifene, keoxifene, toremifene; aromatase inhibitors such as anastrozole, exemestane, letrozole and other aromatase inhibiting 4(5)-imidazoles; gonadotropin-releasing hormone agonists such as nafarelin, triptorelin, goserelin; gonadotropin-releasing hormone antagonists such as degarelix; antiandrogens such as abiraterone, enzalutamide, apalutamide, darolutamide, flutamide, nilutamide, bicalutamide, leuprolide; 5α-reductase inhibitors such as finasteride, dutasteride; and the like. In certain embodiments, combination therapy comprises administration of a hormone or related hormonal agent. In one embodiment, combination therapy comprises administration of enzalutamide.

In some embodiments, one or more of the additional therapeutic agents is an epigenetic modulator. An epigenetic modulator alters an epigenetic mechanism controlling gene expression, and may be, for example, an inhibitor or activator of an epigenetic enzyme. Non-limiting examples of epigenetic modulators include DNA methyltransferase (DNMT) inhibitors, hypomethylating agents, and histone deacetylase (HDAC) inhibitors. In one or more embodiments, the compounds according to this disclosure are combined with DNA methyltransferase (DNMT) inhibitors or hypomethylating agents. Exemplary DNMT inhibitors include decitabine, zebularine and azacitadine. In one or more embodiments, combinations of the compounds according to this disclosure with a histone deacetylase (HDAC) inhibitor is also contemplated. Exemplary HDAC inhibitors include vorinostat, givinostat, abexinostat, panobinostat, belinostat and trichostatin A.

In some embodiments, one or more of the additional therapeutic agents is an ATP-adenosine axis-targeting agent. ATP-adenosine axis-targeting agents alter signaling mediated by adenine nucleosides and nucleotides (e.g., adenosine, AMP, ADP, ATP), for example by modulating the level of adenosine or targeting adenosine receptors. Adenosine and ATP, acting at different classes of receptors, often have opposite effects on inflammation, cell proliferation and cell death. For instance, ATP and other adenine nucleotides have antitumor effects via activation of the PS2Y1 receptor subtype, while accumulation of adenosine in the tumor microenvironment has been shown to inhibit the antitumor function of various immune cells and to augment the immunosuppressive activity of myeloid and regulatory T cells by binding to cell surface adenosine receptors. In certain embodiments, an ATP-adenosine axis-targeting agent is an inhibitor of an ectonucleotidase involved in the conversion of ATP to adenosine or an antagonist of adenosine receptor. Ectonucleotidases involved in the conversion of ATP to adenosine include the ectonucleoside triphosphate diphosphohydrolase 1 (ENTPD1, also known as CD39 or Cluster of Differentiation 39) and the ecto-5′-nucleotidase (NT5E or 5NT, also known as CD73 or Cluster of Differentiation 73). Exemplary small molecule CD73 inhibitors include CB-708, ORIC-533, LY3475070 and quemliclustat. Exemplary anti-CD39 and anti-CD73 antibodies include ES002023, TTX-030, IPH-5201, SRF-617, CPI-006, oleclumab (MEDI9447), NZV930, IPH5301, GS-1423, uliledlimab (TJD5, TJ004309), AB598, and BMS-986179. In one embodiment, the present disclosure contemplates combination of the compounds described herein with a CD73 inhibitor such as those described in WO 2017/120508, WO 2018/067424, WO 2018/094148, and WO 2020/046813. In further embodiments, the CD73 inhibitor is quemliclustat (AB680). Adenosine can bind to and activate four different G-protein coupled receptors: A₁R, A_{2A}R, A_{2B}R, and A₃R. A₂R antagonists include etrumadenant, inupadenant, taminadenant, caffeine citrate, NUV-1182, TT-702, DZD-2269, INCB-106385, EVOEXS-21546, AZD-4635, imaradenant, RVU-330, ciforadenant, PBF-509, PBF-999, PBF-1129, and CS-3005. In some embodiments, the present disclosure contemplates the combination of the compounds described herein with an A2_{A}R antagonist, an A2_{B}R antagonist, or an antagonist of A2_{A}R and A2_{B}R. In some embodiments, the present disclosure contemplates the combination of the compounds described herein with the adenosine receptor antagonists described in WO 2018/136700, WO 2018/204661, WO 2018/213377, or WO 2020/023846. In one embodiment, the adenosine receptor antagonist is etrumadenant.

In some embodiments, one or more of the additional therapeutic agents is a targeted therapy. In one aspect, a targeted therapy may comprise a targeting agent and a drug. The drug may be a chemotherapeutic agent, a radionuclide, a hormone therapy, or another small molecule drug attached to a targeting agent. The targeting agent may be a small molecule, a saccharide (inclusive of oligosaccharides and polysaccharides), an antibody, a lipid, a protein, a peptide, a non-natural polymer, or an aptamer. In some embodiments, the targeting agent is a saccharide (inclusive of oligosaccharides and polysaccharides), a lipid, a protein, or a peptide and the target is a tumor-associated antigen (enriched but not specific to a cancer cell), a tumor-specific antigen (minimal to no expression in normal tissue), or a neo-antigen (an antigen specific to the genome of a cancer cell generated by non-synonymous mutations in the tumor cell genome). In some embodiments, the targeting agent is an antibody and the target is a tumor-associated antigen, a tumor-specific antigen, or a neo-antigen. In some embodiments, the targeted therapy is an antibody-drug conjugate comprising an antibody and a drug, wherein the antibody specifically binds to HER2, HER3, nectin-4, or Trop-2. Specific examples of a targeted therapy comprising an antibody and a drug include but are not limited to patritumab deruxtecan, sacituzumab govitecan-hziy, telisotuzumab vedotin, and trastuzumab deruxtecan. Specific examples include but are not limited to patritumab deruxtecan and telisotuzumab vedotin. In other aspects, a targeted therapy may inhibit or interfere with a specific protein that helps a tumor grow and/or spread. Non-limiting examples of such targeted therapies include signal transduction inhibitors, RAS signaling inhibitors, inhibitors of oncogenic transcription factors, activators of oncogenic transcription factor repressors, angiogenesis inhibitors, immunotherapeutic agents, ATP-adenosine axis-targeting agents, AXL inhibitors, PARP inhibitors, PAK4 inhibitors, PI3K inhibitors, HIF-2a inhibitors, CD39 inhibitors, CD73 inhibitors, A2R antagonists, TIGIT antagonists, and PD-1 antagonists. ATP-adenosine axis-targeting agents are described above, while other agents are described in further detail below.

In some embodiments, one or more of the additional therapeutic agents is a signal transduction inhibitor. Signal transduction inhibitors are agents that selectively inhibit one or more steps in a signaling pathway. Signal transduction inhibitors (STIs) contemplated by the present disclosure include but are not limited to: (i) BCR-ABL kinase inhibitors (e.g., imatinib); (ii) epidermal growth factor receptor tyrosine kinase inhibitors (EGFR TKIs), including small molecule inhibitors (e.g., CLN-081, gefitinib, erlotinib, afatinib, icotinib, and osimertinib), and anti-EGFR antibodies; (iii) inhibitors of the human epidermal growth factor (HER) family of transmembrane tyrosine kinases, e.g., HER-2/neu receptor inhibitors (e.g., trastuzumab) and HER-3 receptor inhibitors; (iv) vascular endothelial growth factor receptor (VEGFR) inhibitors including small molecule inhibitors (e.g., axitinib, sunitinib and sorafenib), VEGF kinase inhibitors (e.g., lenvatinib, cabozantinib, pazopanib, tivozanib, XL092, etc.) and anti-VEGF antibodies (e.g., bevacizumab), and anti-VEGFR antibodies (e.g., ramucirumab); (v) inhibitors of AKT family kinases or the AKT pathway (e.g., rapamycin); (vi) inhibitors of mTOR, such as, for example, everolimus, sirolimus, temsirolimus; (vii) inhibitors of serine/threonine-protein kinase B-Raf (BRAF), such as, for example, vemurafenib, dabrafenib and encorafenib; (viii) inhibitors of rearranged during transfection (RET), including, for example, selpercatinib and pralsetinib; (ix) tyrosine-protein kinase Met (MET) inhibitors (e.g., tepotinib, tivantinib, cabozantinib and crizotinib); (x) anaplastic lymphoma kinase (ALK) inhibitors (e.g., ensartinib, ceritinib, lorlatinib, crizotinib, and brigatinib); (xi) inhibitors of the RAS signaling pathway (e.g., inhibitors of KRAS, HRAS, RAF, MEK, ERK) as described elsewhere herein; (xii) FLT-3 inhibitors (e.g., gilteritinib); (xiii) inhibitors of Trop-2; (xiv) inhibitors of the JAK/STAT pathway, e.g., JAK inhibitors including tofacitinib and ruxolitinib, or STAT inhibitors such as napabucasin; (xv) inhibitors of NF-κB; (xvi) cell cycle kinase inhibitors (e.g., flavopiridol); (xvii) phosphatidyl inositol kinase (PI3K) inhibitors; (xiii) protein kinase B (AKT) inhibitors (e.g., capivasertib, miransertib); (xviii) platelet-derived growth factor receptor (PDGFR) inhibitors (e.g., imatinib, sunitinib, regorafenib, avapritinib, lenvatinib, nintedanib, famitinib, ponatinib, axitinib, repretinib, etc.); (xix) insulin-like growth factor receptor (IGFR) inhibitors (e.g., erlotinib, afatinib, gefitinib, osimertinib, dacomitinib); (xx) fibroblast growth factor receptor (FGFR) inhibitors (e.g., futibatinib, erdafitinib, pemigatinib); and (xxi) receptor tyrosine kinase KIT inhibitors (e.g., imatinib, sorafenib, sunitinib, masitinib, repretinib, avapritinib). In one or more embodiments, the additional therapeutic agent comprises an inhibitor of EGFR, VEGFR, HER-2, HER-3, BRAF, RET, MET, ALK, RAS (e.g., KRAS, MEK, ERK), FLT-3, JAK, STAT, NF-κB, PI3K, AKT, FGFR, KIT, or any combinations thereof.

In some embodiments, one or more of the additional therapeutic agents is a RAS signaling inhibitor. Oncogenic mutations in the RAS family of genes, e.g., HRAS, KRAS, and NRAS, are associated with a variety of cancers. For example, mutations of G12C, G12D, G12V, G12A, G13D, Q61H, G13C and G12S, among others, in the KRAS family of genes have been observed in multiple tumor types. Direct and indirect inhibition strategies have been investigated for the inhibition of mutant RAS signaling. Indirect inhibitors target effectors other than RAS in the RAS signaling pathway, and include, but are not limited to, inhibitors of RAF, MEK, ERK, PI3K, PTEN, SOS (e.g., SOS1), mTORC1, SHP2 (PTPN11), and AKT. Non-limiting examples of indirect inhibitors under development include RMC-4630, RMC-5845, RMC-6291, RMC-6236, JAB-3068, JAB-3312, TNO155, RLY-1971, BI1701963. Direct inhibitors of RAS mutants have also been explored, and generally target the KRAS-GTP complex or the KRAS-GDP complex. Exemplary direct RAS inhibitors under development include, but are not limited to, sotorasib (AMG510), adagrasib (MRTX849), mRNA-5671 and ARS1620. In some embodiments, the one or more RAS signaling inhibitors are selected from the group consisting of RAF inhibitors, MEK inhibitors, ERK inhibitors, PI3K inhibitors, PTEN inhibitors, SOS1 inhibitors, mTORC1 inhibitors, SHP2 inhibitors, and AKT inhibitors. In other embodiments the one or more RAS signaling inhibitors directly inhibit RAS mutants.

In some embodiments one or more of the additional therapeutic agents is an inhibitor of a phosphatidylinositol 3-kinase (PI3K), particularly an inhibitor of the PI3Kγ isoform. PI3Kγ inhibitors can stimulate an anti-cancer immune response through the modulation of myeloid cells, such as by inhibiting suppressive myeloid cells, dampening immune-suppressive tumor-infiltrating macrophages or by stimulating macrophages and dendritic cells to make cytokines that contribute to effective T cell responses thereby decreasing cancer development and spread. Exemplary PI3Kγ inhibitors include copanlisib, duvelisib, AT-104, ZX-101, tenalisib, eganelisib, SF-1126, AZD3458, and pictilisib. In some embodiments, the compounds according to this disclosure are combined with one or more PI3Kγ inhibitors described in WO 2020/0247496A1.

In some embodiments, one or more of the additional therapeutic agents is an inhibitor of arginase. Arginase has been shown to be either responsible for or participate in inflammation-triggered immune dysfunction, tumor immune escape, immunosuppression and immunopathology of infectious disease. Exemplary arginase compounds include CB-1158 and OAT-1746. In some embodiments, the compounds according to this disclosure are combined with one or more arginase inhibitors described in WO/2019/173188 and WO 2020/102646.

In some embodiments, one or more of the additional therapeutic agents is an inhibitor of an oncogenic transcription factor or an activator of an oncogenic transcription factor repressor. Suitable agents may act at the expression level (e.g., RNAi, siRNA, etc.), through physical degradation, at the protein/protein level, at the protein/DNA level, or by binding in an activation/inhibition pocket. Non-limiting examples include inhibitors of one or more subunit of the MLL complex (e.g., HDAC, DOT1L, BRD4, Menin, LEDGF, WDR5, KDM4C (JMJD2C) and PRMT1), inhibitors of hypoxia-inducible factor (HIF) transcription factor, and the like.

In some embodiments, one or more of the additional therapeutic agents is an inhibitor of a hypoxia-inducible factor (HIF) transcription factor, particularly HIF-2a. Exemplary HIF-2a inhibitors include belzutifan, ARO-HIF2, PT-2385, AB521, NKT-2152, DFF332, and those described in WO 2021113436, WO 2021188769, and WO 2023077046. In some embodiments, the HIF-2a inhibitor is AB521.

In some embodiments, one or more of the additional therapeutic agents is an inhibitor of anexelekto (AXL). The AXL signaling pathway is associated with tumor growth and metastasis, and is believed to mediate resistance to a variety of cancer therapies. There are a variety of AXL inhibitors under development that also inhibit other kinases in the TAM family (i.e., TYRO3, MERTK), as well as other receptor tyrosine kinases including MET, FLT3, RON and AURORA, among others. Exemplary multikinase inhibitors include sitravatinib, rebastinib, glesatinib, gilteritinib, merestinib, cabozantinib, foretinib, BMS777607, LY2801653, S49076, and RXDX-106. AXL specific inhibitors have also been developed, e.g., small molecule inhibitors including DS-1205, SGI-7079, SLC-391, dubermatinib, bemcentinib, DP3975, and AB801; anti-AXL antibodies such as ADCT-601; and antibody drug conjugates (ADCs) such as BA3011. Another strategy to inhibit AXL signaling involves targeting AXL's ligand, GAS6. For example, batiraxcept is under development as is a Fc fusion protein that binds the GAS6 ligand thereby inhibiting AXL signaling. In some embodiments, the compounds according to this disclosure are combined with one or more AXL inhibitors described in WO2022246177, WO2022246179, or WO2024006726. In some embodiments, the AXL inhibitor is AB801.

In some embodiments, one or more of the additional therapeutic agents is an inhibitor of p21-activated kinase 4 (PAK4). PAK4 overexpression has been shown across a variety of cancer types, notably including those resistant to PD-1 therapies.

In some embodiments, one or more of the additional therapeutic agents is (i) an agent that inhibits the enzyme poly (ADP-ribose) polymerase (e.g., olaparib, niraparib and rucaparib, etc.); (ii) an inhibitor of the Bcl-2 family of proteins (e.g., venetoclax, navitoclax, etc.); (iii) an inhibitor of MCL-1; (iv) an inhibitor of the CD47-SIRPα pathway (e.g., an anti-CD47 antibody); (v) an isocitrate dehydrogenase (IDH) inhibitor, e.g., IDH-1 or IDH-2 inhibitor (e.g., ivosidenib, enasidenib, etc.).

In some embodiments, one or more of the additional therapeutic agents is an immunotherapeutic agent. Immunotherapeutic agents treat a disease by stimulating or suppressing the immune system. Immunotherapeutic agents useful in the treatment of cancers typically elicit or amplify an immune response to cancer cells. Non-limiting examples of suitable immunotherapeutic agents include: immunomodulators; cellular immunotherapies; vaccines; gene therapies; ATP-adenosine axis-targeting agents; immune checkpoint modulators; and certain signal transduction inhibitors. ATP-adenosine axis-targeting agents and signal transduction inhibitors are described above. Immunomodulators, cellular immunotherapies, vaccines, gene therapies, and immune checkpoint modulators are described further below.

In some embodiments, one or more of the additional therapeutic agents is an immunotherapeutic agent, more specifically a cytokine or chemokine, such as, IL-1, IL-2, IL-12, IL-18, ELC/CCL19, SLC/CCL21, MCP-1, IL-4, TNF, IL-15, MDC, IFNα, IFNβ, IFNγ, M-CSF, IL-3, GM-CSF, IL-13, and anti-IL-10; bacterial lipopolysaccharides (LPS); an organic or inorganic adjuvant that activates antigen-presenting cells and promote the presentation of antigen epitopes on major histocompatibility complex molecules agonists including, but not limited to Toll-like receptor (TLR) agonists, antagonists of the mevalonate pathway, agonists of STING; indoleamine 2,3-dioxygenase 1 (IDO1) inhibitors and immune-stimulatory oligonucleotides, as well as other T cell adjuvants.

In some embodiments, one or more of the additional therapeutic agents is an immunotherapeutic agent, more specifically a cellular therapy. Cellular therapies are a form of treatment in which viable cells are administered to a subject. In certain embodiments, one or more of the additional therapeutic agents is a cellular immunotherapy that activates or suppresses the immune system. Cellular immunotherapies useful in the treatment of cancers typically elicit or amplify an immune response. The cells can be autologous or allogenic immune cells (e.g., monocytes, macrophages, dendritic cells, NK cells, T cells, etc.) collected from one or more subject. Alternatively, the cells can be “(re)programmed” allogenic immune cells produced from immune precursor cells (e.g., lymphoid progenitor cells, myeloid progenitor cells, common dendritic cell precursor cells, stem cells, induced pluripotent stem cells, etc.). In some embodiments, such cells may be an expanded subset of cells with distinct effector functions and/or maturation markers (e.g., adaptive memory NK cells, tumor infiltrating lymphocytes, immature dendritic cells, monocyte-derived dendritic cells, plasmacytoid dendritic cells, conventional dendritic cells (sometimes referred to as classical dendritic cells), M1 macrophages, M2 macrophages, etc.), may be genetically modified to target the cells to a specific antigen and/or enhance the cells' anti-tumor effects (e.g., engineered T cell receptor (TCR) cellular therapies, chimeric antigen receptor (CAR) cellular therapies, lymph node homing of antigen-loaded dendritic cells, etc.), may be engineered to express of have increased expression of a tumor-associated antigen, or may be any combination thereof. Non-limiting types of cellular therapies include CAR-T cell therapy, CAR-NK cell therapy, TCR therapy, and dendritic cell vaccines. Exemplary cellular immunotherapies include sipuleucel-T, tisagenlecleucel, lisocabtagene maraleucel, idecabtagene vicleucel, brexucabtagene autoleucel, and axicabtagene ciloleucel, as well as CTX110, JCAR015, JCAR017, MB-CART19.1, MB-CART20.1, MB-CART2019.1, UniCAR02-T-CD123, BMCA-CAR-T, JNJ-68284528, BNT211, and NK-92/5.28.z.

In some embodiments, one or more of the additional therapeutic agents is an immunotherapeutic agent, more specifically a gene therapy. Gene therapies comprise recombinant nucleic acids administered to a subject or to a subject's cells ex vivo in order to modify the expression of an endogenous gene or to result in heterologous expression of a protein (e.g., small interfering RNA (siRNA) agents, double-stranded RNA (dsRNA) agents, micro RNA (miRNA) agents, viral or bacterial gene delivery, etc.), as well as gene editing therapies that may or may not comprise a nucleic acid component (e.g., meganucleases, zinc finger nucleases, TAL nucleases, CRISPR/Cas nucleases, etc.), oncolytic viruses, and the like. Non-limiting examples of gene therapies that may be useful in cancer treatment include Gendicine® (rAd-p53), Oncorine® (rAD5-H101), talimogene laherparepvec, Mx-dnG1, ARO-HIF2 (Arrowhead), quaratusugene ozeplasmid (Immunogene), CTX110 (CRISPR Therapeutics), CTX120 (CRISPR Therapeutics), and CTX130 (CRISPR Therapeutics).

In some embodiments, one or more of the additional therapeutic agents is an immunotherapeutic agent, more specifically an agent that modulates an immune checkpoint. Immune checkpoints are a set of inhibitory and stimulatory pathways that directly affect the function of immune cells (e.g., B cells, T cells, NK cells, etc.). Immune checkpoints engage when proteins on the surface of immune cells recognize and bind to their cognate ligands. The present invention contemplates the use of compounds described herein in combination with agonists of stimulatory or co-stimulatory pathways and/or antagonists of inhibitory pathways. Agonists of stimulatory or co-stimulatory pathways and antagonists of inhibitory pathways may have utility as agents to overcome distinct immune suppressive pathways within the tumor microenvironment, inhibit T regulatory cells, reverse/prevent T cell anergy or exhaustion, trigger innate immune activation and/or inflammation at tumor sites, or combinations thereof.

In some embodiments, one or more of the additional therapeutic agents is an immune checkpoint inhibitor. As used herein, the term “immune checkpoint inhibitor” refers to an antagonist of an inhibitory or co-inhibitory immune checkpoint. The terms “immune checkpoint inhibitor”, “checkpoint inhibitor” and “CPI” may be used herein interchangeably. Immune checkpoint inhibitors may antagonize an inhibitory or co-inhibitory immune checkpoint by interfering with receptor-ligand binding and/or altering receptor signaling. Examples of immune checkpoints (ligands and receptors), some of which are selectively upregulated in various types of cancer cells, that can be antagonized include PD-1 (programmed cell death protein 1); PD-L1 (PD1 ligand); BTLA (B and T lymphocyte attenuator); CTLA-4 (cytotoxic T-lymphocyte associated antigen 4); TIM-3 (T cell immunoglobulin and mucin domain containing protein 3); LAG-3 (lymphocyte activation gene 3); TIGIT (T cell immunoreceptor with Ig and ITIM domains); CD276 (B7-H3), PD-L2, Galectin 9, CEACAM-1, CD69, Galectin-1, CD113, GPR56, VISTA, 2B4, CD48, GARP, PD1H, LAIR1, TIM-1, and TIM-4, and Killer Inhibitory Receptors, which can be divided into two classes based on their structural features: i) killer cell immunoglobulin-like receptors (KIRs), and ii) C-type lectin receptors (members of the type II transmembrane receptor family). Also contemplated are other less well-defined immune checkpoints that have been described in the literature, including both receptors (e.g., the 2B4 (also known as CD244) receptor) and ligands (e.g., certain B7 family inhibitory ligands such B7-H3 (also known as CD276) and B7-H4 (also known as B7-S1, B7x and VCTN1)). [See Pardoll, (April 2012) Nature Rev. Cancer 12:252-64].

In some embodiments, an immune checkpoint inhibitor is a CTLA-4 antagonist. In further embodiments, the CTLA-4 antagonist can be an antagonistic CTLA-4 antibody. Suitable antagonistic CTLA-4 antibodies include, for example, monospecific antibodies such as ipilimumab or tremelimumab, as well as bispecific antibodies such as MEDI5752 and KN046.

In some embodiments, an immune checkpoint inhibitor is a PD-1 antagonist. In further embodiments, the PD-1 antagonist can be an antagonistic PD-1 antibody, small molecule or peptide. Suitable antagonistic PD-1 antibodies include, for example, monospecific antibodies such as balstilimab, budigalimab, camrelizumab, cosibelimab, dostarlimab, cemiplimab, ezabenlimab, MEDI-0680 (AMP-514; WO2012/145493), nivolumab, pembrolizumab, pidilizumab (CT-011), pimivalimab, retifanlimab, sasanlimab, spartalizumab, sintilimab, tislelizumab, toripalimab, and zimberelimab; as well as bi-specific antibodies such as LY3434172, IBI321, ivonescimab, rilvegostomig, tebotelimab, and tobemstomig. In still further embodiments, the PD-1 antagonist can be a recombinant protein composed of the extracellular domain of PD-L2 (B7-DC) fused to the Fc portion of IgGl (AMP-224). In certain embodiments, an immune checkpoint inhibitor is zimberelimab.

In some embodiments, an immune checkpoint inhibitor is a PD-L1 antagonist. In further embodiments, the PD-L1 antagonist can be an antagonistic PD-L1 antibody. Suitable antagonistic PD-L1 antibodies include, for example, monospecific antibodies such as avelumab, atezolizumab, durvalumab, BMS-936559, and envafolimab as well as bi-specific antibodies such as LY3434172 and KN046.

In some embodiments, an immune checkpoint inhibitor is a TIGIT antagonist. In further embodiments, the TIGIT antagonist can be an antagonistic TIGIT antibody. Suitable antagonistic anti-TIGIT antibodies include monospecific antibodies such as AGEN1327, AB308 (WO2021247591), BMS 986207, COM902, domvanalimab, belrestotug, etigilimab, IBI-929, JS006, dargistotug, ociperlimab, SEA-TGT, tiragolumab, vibostolimab; as well as bi-specific antibodies such as AGEN1777 and rilvegostomig. In certain embodiments, an immune checkpoint inhibitor is an antagonistic anti-TIGIT antibody disclosed in WO2017152088 or WO2021247591. In certain embodiments, an immune checkpoint inhibitor is domvanalimab or AB308.

In some embodiments, an immune checkpoint inhibitor is a LAG-3 antagonist. In further embodiments, the LAG-3 antagonist can be an antagonistic LAG-3 antibody. Suitable antagonistic LAG-3 antibodies include, for example, BMS-986016 (WO10/19570, WO14/08218), or IMP-731 or IMP-321 (WO08/132601, WO09/44273).

In certain embodiments, an immune checkpoint inhibitor is a B7-H3 antagonist. In further embodiments, the B7-H3 antagonist is an antagonistic B7-H3 antibody. Suitable antagonist B7-H3 antibodies include, for example, enoblituzumab (WO11/109400), omburtumab, DS-7300a, ABBV-155, and SHR-A1811.

In some embodiments, an immune checkpoint inhibitor is a TIM-3 antagonist. In further embodiments, the TIM-3 antagonist can be an antagonistic TIM-3 antibody. Suitable antagonistic TIM-3 antibodies include, for example, sabatolimab, BMS-986258, and RG7769/RO7121661.

In some embodiments, one or more of the additional therapeutic agents activates a stimulatory or co-stimulatory immune checkpoint. Examples of stimulatory or co-stimulatory immune checkpoints (ligands and receptors) include B7-1, B7-2, CD28, 4-1BB (CD137), 4-1BBL, ICOS, ICOS-L, OX40, OX40L, GITR, GITRL, CD70, CD27, CD40, DR3 and CD2.

In some embodiments, an agent that activates a stimulatory or co-stimulatory immune checkpoint is a CD137 (4-1BB) agonist. In further embodiments, the CD137 agonist can be an agonistic CD137 antibody. Suitable CD137 antibodies include, for example, urelumab and utomilumab (WO12/32433). In some embodiments, an agent that activates a stimulatory or co-stimulatory immune checkpoint is a GITR agonist. In further embodiments, the GITR agonist can be an agonistic GITR antibody. Suitable GITR antibodies include, for example, BMS-986153, BMS-986156, TRX-518 (WO06/105021, WO09/009116) and MK-4166 (WO11/028683). In some embodiments, an agent that activates a stimulatory or co-stimulatory immune checkpoint is an OX40 agonist. In further embodiments, the OX40 agonist can be an agonistic OX40 antibody. Suitable OX40 antibodies include, for example, MEDI-6383, MEDI-6469, MEDI-0562, PF-04518600, GSK3174998, BMS-986178, and MOXR0916. In some embodiments, an agent that activates a stimulatory or co-stimulatory immune checkpoint is a CD40 agonist. In further embodiments, the CD40 agonist can be an agonistic CD40 antibody. In some embodiments, an agent that activates a stimulatory or co-stimulatory immune checkpoint is a CD27 agonist. In further embodiments, the CD27 agonist can be an agonistic CD27 antibody. Suitable CD27 antibodies include, for example, varlilumab.

In some embodiments, one or more of the additional therapies is an immunotherapeutic agent, more specifically an intracellular signaling molecule that influences immune cell function. For example, one or more of the additional therapies may be an inhibitor of hematopoietic progenitor kinase 1 (HPK1). HPK1 is serine/threonine kinase that functions as a negative regulator of activation signals generated by the T cell antigen receptor. As another example, one or more of the additional therapies may be an inhibitor of diacylglycerol kinase (DGK). In some embodiments, the inhibitor is a small molecule. Non-limiting examples of small molecule HPK1 inhibitors in clinical development include NDI-101150, PRJ1-3024, PF-07265028, GRC 54276, CFI-402411 and BGB-15025. Non-limiting examples of small molecule DGK inhibitors include ASP1570, BAY2965501.

In some embodiments, one or more of the additional therapeutic agents is an agent that inhibits or depletes immune-suppressive immune cells. For example, to inhibit or deplete immunosuppressive macrophages or monocytes the agent may be CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264), or CSF-1R antagonists disclosed in WO14/036357. In another example, to inhibit or deplete regulatory T cells (Tregs), the agent may be an anti-CD25 antibody or immunotoxin targeting CD25.

In some embodiments, each additional therapeutic agent can independently be a chemotherapeutic agent, a radiopharmaceutical, a hormone therapy, an epigenetic modulator, a targeted agent, an immunotherapeutic agent, a cellular therapy, or a gene therapy. For example, in one embodiment, the present disclosure contemplates the use of the compounds described herein in combination with one or more chemotherapeutic agent and optionally one or more additional therapeutic agents, wherein each additional therapeutic agent is independently a radiopharmaceutical, a hormone therapy, a targeted agent, an immunotherapeutic agent, a cellular therapy, or a gene therapy. In another embodiment, the present disclosure contemplates the use of the compounds described herein in combination with one or more chemotherapeutic agent and optionally one or more additional therapeutic agents, wherein each additional therapeutic agent is independently a targeted agent, an immunotherapeutic agent, or a cellular therapy. In another embodiment, the present disclosure contemplates the use of the compounds described herein in combination with one or more immunotherapeutic agents and optionally one or more additional therapeutic agent, wherein each additional therapeutic agent is independently a radiopharmaceutical, a hormone therapy, a targeted agent, a chemotherapeutic agent, a cellular therapy, or a gene therapy. In another embodiment, the present disclosure contemplates the use of the compounds described herein in combination with one or more immunotherapeutic agents and optionally one or more additional therapeutic agents, wherein each additional therapeutic agent is independently a chemotherapeutic agent, a targeted agent, or a cellular therapy. In another embodiment, the present disclosure contemplates the use of the compounds described herein in combination with one or more immune checkpoint inhibitors and/or one or more ATP-adenosine axis-targeting agents, and optionally one or more additional therapeutic agents, wherein each additional therapeutic agent is independently a chemotherapeutic agent, a targeted agent, an immunotherapeutic agent, or a cellular therapy. In further embodiments of the above (a) the targeted agent can be a PI3K inhibitor, an arginase inhibitor, a HIF2a inhibitor, an AXL inhibitor, or a PAK4 inhibitor; (b) the immunotherapeutic agent is an ATP-adenosine axis-targeting agent or an immune checkpoint inhibitor; (c) the ATP-adenosine axis-targeting agent is an A2_{A}R and/or A2_{B}R antagonist, a CD73 inhibitor, or a CD39 inhibitor; (d) the ATP-adenosine axis-targeting agent is etrumadenant, quemliclustat, or AB598; (e) the immunotherapeutic agent is an anti-PD-1 antagonist antibody or an anti-TIGIT antagonist antibody; (f) the immunotherapeutic agent is zimberelimab, domvanalimab, or AB308; or (g) any combination thereof. In still further embodiments of the above, the present disclosure contemplates the use of the compounds described herein in combination with domvanalimab, etrumadenant, quemliclustat, zimberelimab, AB308, AB521, AB598, AB610, AB801 or any combination thereof.

Selection of the additional therapeutic agent(s) may be informed by current standard of care for a particular cancer and/or mutational status of a subject's cancer and/or stage of disease. Detailed standard of care guidelines are published, for example, by National Comprehensive Cancer Network (NCCN). See, for instance, NCCN Colon Cancer v1.2022, NCCN Hepatobiliary Cancer v1.2022, NCCN Kidney Cancer, v3.2022, NCCN NSCLC v3.2022, NCCN Pancreatic Adenocarcinoma v1.2022, NCCN Esophageal and Esophagogastric Junction Cancers v2.2022, NCCN Gastric Cancer v2.2022, Cervical Cancer v1.2022, Ovarian Cancer/Fallopian Tube Cancer/Primary Peritoneal Cancer v1.2022.

### Experimental

The following examples are put forth so as to provide those of ordinary skill in the art with a complete disclosure and description of how to make and use the present disclosure, and are not intended to limit the scope of what the inventors regard as their invention. Efforts have been made to ensure accuracy with respect to numbers used (e.g., amounts, temperature, etc.), but some experimental errors and deviations should be accounted for.

All reactions were performed using a Teflon-coated magnetic stir bar at the indicated temperature and were conducted under an inert atmosphere when stated. Purchased starting materials and reagents were generally used as received. Reactions were monitored by TLC (silica gel 60 with fluorescence F254, visualized with a short wave/long wave UV lamp) and/or LCMS (AGILENT® 1100 or 1200 series LCMS with UV detection at 254 or 280 nm using a binary solvent system [0.1% formic acid in MeCN/0.1% formic acid in H₂O] using one of the following columns: AGILENT® Eclipse Plus C18 [3.5 μm, 4.6 mm i.d.×100 mm], WATERS™ XSelect HSS C18 [3.5 μm, 2.1 mm i.d.×75 mm]). Flash chromatography was conducted on silica gel using an automated system (COMBIFLASH® RF+ manufactured by Teledyne ISCO), with detection wavelengths of 254 and 280 nm, and optionally equipped with an evaporative light scattering detector. Reverse phase preparative HPLC was conducted on an AGILENT® 1260 or 1290 Infinity series HPLC. Samples were eluted using a binary solvent system (MeCN/H₂O with an acid modifier as needed—for example 0.1% TFA or 0.1% formic acid) with gradient elution on a Gemini C18 110 Å column (21.2 mm i.d.×250 mm) with variable wavelength detection. Final compounds obtained through preparative HPLC were concentrated through lyophilization. All assayed compounds were purified to ≥95% purity as determined by ¹H NMR or LCMS (AGILENT® 1100 or 1200 series LCMS with UV detection at 254 or 280 nm using a binary solvent system [0.1% formic acid in MeCN/0.1% formic acid in H₂O] using one of the following columns: AGILENT® Eclipse Plus C18 [3.5 μm, 4.6 mm i.d.×100 mm], WATERS™ XSelect HSS C18 [3.5 μm, 2.1 mm i.d.×75 mm]). ¹H NMR spectra were recorded on a Varian 400 MHz NMR spectrometer equipped with an Oxford AS400 magnet or a BRUKER® AVANCE NEO 400 MHz NMR. Chemical shifts (6) are reported as parts per million (ppm) relative to residual undeuterated solvent as an internal reference. The abbreviations s, br s, d, t, q, dd, dt, ddd, and m stand for singlet, broad singlet, doublet, triplet, quartet, doublet of doublets, doublet of triplets, doublet of doublet of doublets, and multiplet, respectively.

Unless indicated otherwise, temperature is in degrees Celsius (° C.), and pressure is at or near atmospheric. Standard abbreviations are used, including the following: rt=room temperature; min(s)=minute(s); h=hour(s); mg=milligram; g=gram; kg=kilogram; L=microliter; ml or mL=milliliter; M=molar; mol=mole; mmol=millimole; N=normality; sat.=saturated; aq.=aqueous; psi=pounds per square inch; calcd=calculated; equiv.=equivalents; AcOH=acetic acid; HCO₂H=formic acid; H₂SO₄=sulfuric acid; MTBE=methyl tert-butyl ether; DCM=dichloromethane; DCE=1,2-dichloroethane; PhMe=toluene; THF=tetrahydrofuran; EtOAc=ethyl acetate; TFA=trifluoroacetic acid; MeCN or CH₃CN=acetonitrile; ClCOOEt=ethyl chloroformate; NMP=N-methyl-2-pyrrolidone; DMF=N,N-dimethylformamide; DMSO=dimethyl sulfoxide; DMA=dimethylacetamide; CHCl₃=chloroform; CDCl₃=deuterated chloroform; MeOH=methanol; CD₃OD=deuterated methanol; N₂=nitrogen gas; H₂O₂=hydrogen peroxide; T₃P=1-propanephosphonic anhydride solution; DIPEA=N,N-diisopropylethylamine; DBAD=di-tert-butylazodicarboxylate; DMEDA=N,N-dimethylethane-1,2-diamine; SEM-Cl=2-(trimethylsilyl)ethoxymethyl chloride; PMBCl=4-methoxybenzyl chloride; TBAF=tetrabutylammonium fluoride; MeI or CH₃I=iodomethane; HMPA=hexamethylphosphoramide; Et₃N=triethylamine; NaBH(OAc)₃=sodium triacetoxyborohydride; NaBH₄=sodium borohydride; NaBH₃CN=sodium cyanoborohydride; NaOAc=sodium acetate; NaOMe=sodium methoxide; NH₄Cl=ammonium chloride; NH₄OH=ammonium hydroxide; NH₃=ammonia; Na₂SO₄=sodium sulfate; NaHCO₃=sodium bicarbonate; NaH=sodium hydride; NaOH=sodium hydroxide; Na₂S₂O₃=sodium thiosulfate; KI=potassium iodide; K₂CO₃=potassium carbonate; K₃PO₄=potassium phosphate; MgSO₄=magnesium sulfate; LiOH·H₂O=lithium hydroxide monohydrate; nBuLi=n-butyllithium; LiTMP=2,2,6,6-tetramethylpiperidine lithium; LDA=lithium diisopropylamide; t-BuONO=tert-butyle nitrile; PhNTf₂=bis(trifluoromethanesulfonyl)aniline; CuI=copper iodide; Cu(OAc)₂=copper(II) acetate; Ag₂CO₃=silver carbonate; CuBr₂=copper(II) bromide; FeCl₃=iron(III) chloride; SnCl₂=tin(II) chloride; MnO₂=manganese dioxide; HCl=hydrochloric acid; TMSN₃=trimethylsilyl azide; TMSCl=trimethylsilyl chloride; MsCl=methanesulfonyl chloride; DMP=Dess-Martin periodinane; NBS=N-bromosuccinimide; HATU=1-[bis(dimethylamino)methylene]-1H-1,2,3-triazolo[4,5-b]pyridinium 3-oxide hexafluorophosphate; Cs₂CO₃=cesium carbonate; AgOTf=silver trifluoromethanesulfonate; Ti(Oi-Pr)₄=titanium(IV) isopropoxide; XantPhos=(9,9-dimethyl-9H-xanthene-4,5-diyl)bis(diphenylphosphane); Pd(dppf)Cl₂=[1,1′-bis(diphenylphosphino)ferrocene]palladium(II) dichloride; Xphos Pd G3=(2-dicyclohexylphosphino-2′,4′,6′-triisopropyl-1,1′-biphenyl)[2-(2′-amino-1,1′-biphenyl)]palladium(II) methanesulfonate; Xphos=dicyclohexyl[2′,4′,6′-tris(propan-2-yl)[1,1′-biphenyl]-2-yl]phosphane; tBuXphos Pd G3=[(2-di-tert-butylphosphino-2′,4′,6′-triisopropyl-1,1′-biphenyl)-2-(2′-amino-1,1′-biphenyl)] palladium(II) methanesulfonate; Pd(dtBPF)Cl₂=[1,1′-bis(di-tert-butylphosphino)ferrocene]dichloropalladium(II); Pd(OAc)₂=palladium(II) acetate; Pd(PPh₃)₄=tetrakis(triphenylphosphine)palladium(0); PCy₃=tricyclohexylphosphine; Zn(CN)₂=zinc cyanide; RuCp*(COD)Cl=chloro(1,5-cyclooctadiene)(pentamethylcyclopentadienyl)ruthenium; [Cp*RuCl]₄=tetrachlorotetrakis(pentamethylcyclopentadienyl)tetraruthenium; MHz=megahertz; Hz=hertz; ppm=parts per million; ESI MS=electrospray ionization mass spectrometry; LCMS=liquid chromatography-mass spectrometry; NMR=nuclear magnetic resonance; HPLC=high pressure liquid chromatography.

### Example 1: 4-[2-(2-{[(S)-3-methyl-1-piperidyl]methyl}-4-cyclopropyl-7-oxo-1,6-dihydro-1,6-diaza-6-indenyl)-6-chloro-4-pyridyl]-3-(4-methyl-4H-1,2,4-triazol-3-yl)benzonitrile

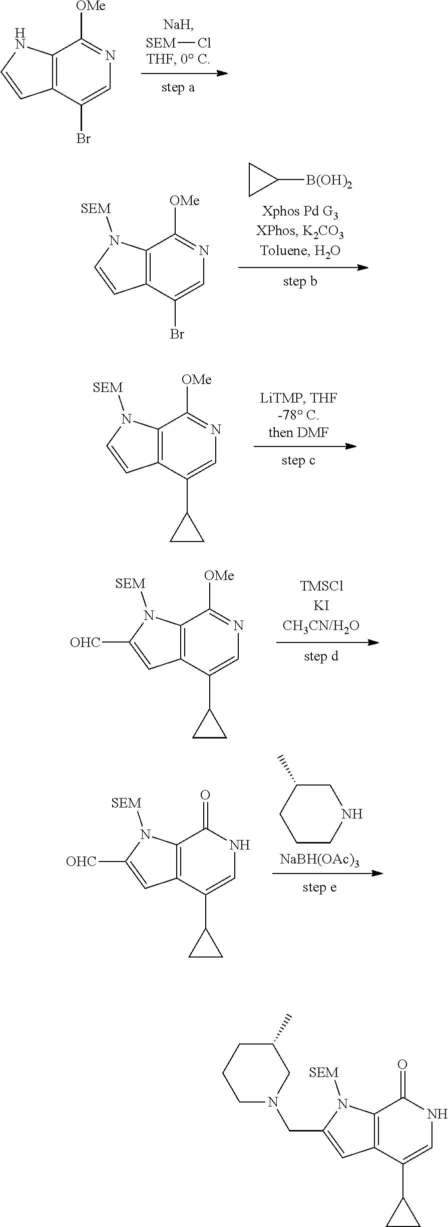

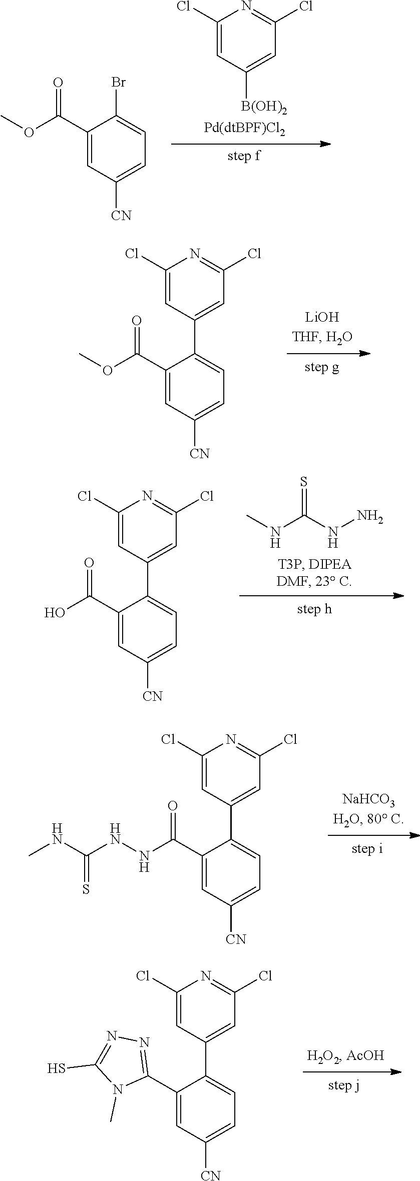

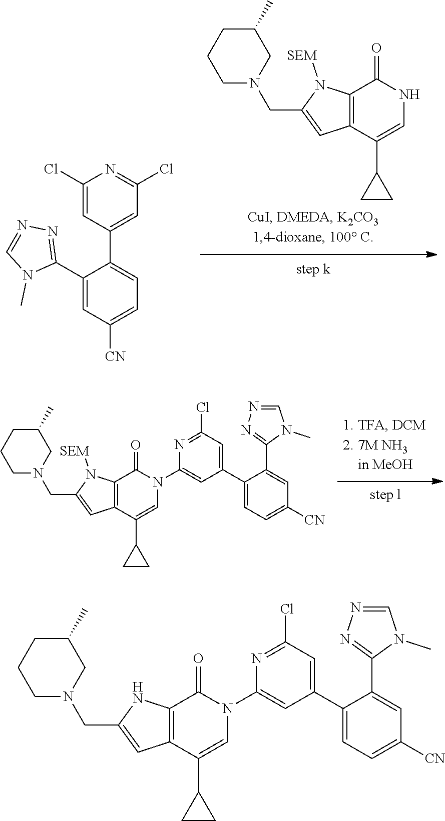 [SMILES: Cn1cnnc1-c1cc(C#N)ccc1-c1cc(Cl)nc(Cl)c1 ; C[C@H]1CCCN(Cc2cc3c(C4CC4)c[nH]c(=O)c3n2COCC[Si](C)(C)C)C1 ; C[C@H]1CCCN(Cc2cc3c(C4CC4)cn(-c4cc(-c5ccc(C#N)cc5-c5nncn5C)cc(Cl)n4)c(=O)c3n2COCC[Si](C)(C)C)C1 ; C[C@H]1CCCN(Cc2cc3c(C4CC4)cn(-c4cc(-c5ccc(C#N)cc5-c5nncn5C)cc(Cl)n4)c(=O)c3[nH]2)C1]

Step a: To a solution of 4-bromo-7-methoxy-1H-pyrrolo[2,3-c]pyridine (8.0 g, 35.24 mmol, 1.0 equiv.) in THE (100 ml, 0.3 M) NaH (2.54 g, 105.72 mmol, 3.0 equiv.) was added followed by SEM-Cl (6.46 g, 38.76 mmol, 1.1 equiv.) at 0° C. The resulting mixture was stirred at rt for 2 h. The reaction mixture was quenched with water, the organic phase was separated, and the aqueous layer was extracted with EtOAc. The combined organic phase was dried over Na₂SO₄, concentrated, and the crude residue was purified by column chromatography (SiO₂, EtOAc in hexanes, 20 to 80%) to give 2-[(4-bromo-7-methoxypyrrolo[2,3-c]pyridin-1-yl)methoxy]ethyl-trimethylsilane.

Step b: The product of step a (6.60 g, 18.44 mmol, 1.0 equiv.), cyclopropylboronic acid (2.0 g, 23.04 mmol, 1.25 equiv.) and K₂CO₃ (7.60 g, 55.31 mmol, 3.0 equiv.) were dissolved in toluene/water mixture (60 mL/12 mL, 0.25 M). The mixture was purged for 2 mins through nitrogen inlet tube. Then Xphos Pd G3 (780 mg, 0.9218 mmol, 0.05 equiv.) and Xphos (703 mg, 1.4749 mmol, 0.08 equiv.) were added. The mixture was stirred at 90° C. for 12 h. After cooling down to rt the reaction mixture was quenched with water, the organic phase was separated, and the aqueous layer was extracted with EtOAc. The combined organic phase was dried over Na₂SO₄ and concentrated under reduced pressure. The crude residue was purified by column chromatography (SiO₂, EtOAc in hexanes, 20 to 80%) to give 2-[(4-cyclopropyl-7-methoxypyrrolo[2,3-c]pyridin-1-yl)methoxy]ethyl-trimethylsilane.

Step c: To a solution of 2,2,6,6-tetramethylpiperidine (1.53 ml, 9.0 mmol, 1.6 equiv.) in THE (50 mL, 0.18 M) n-butyllithium (3.6 ml, 8.9826 mmol, 1.6 equiv., 2.5 M in hexanes) was added dropwise over 3 min at −78° C. The resulting mixture was stirred at −78° C. for an additional 5 min. Then the product of step b (1.8 g, 5.6 mmol, 1.0 equiv.) was added dropwise at −78° C. in THE (10 mL). The resulting mixture was stirred at −78° C. for 1 h. Then DMF (0.8 ml, 10.1 mmol, 1.8 equiv.) was added to the mixture, and the resulting solution was stirred for 30 min at −78° C. The reaction was quenched with sat. aq. NH₄Cl solution, the organic phase was separated, and the aqueous layer was extracted with EtOAc. The combined organic phase was dried over Na₂SO₄ and concentrated to dryness under reduced pressure to produce crude aldehyde product that was used for the next step without purification.

Step d: To a solution of aldehyde product from step c (1.95 g, 5.6 mmol, 1.0 equiv.) and KI (1.50 g, 8.96 mmol, 1.6 equiv.) in CH₃CN (50 mL, 0.1 M) TMSCl (977 mg, 8.96 mmol, 1.6 equiv.) was added dropwise at rt. After water (0.1 ml) was added the reaction mixture was stirred at rt for 12 h. Once LCMS analysis indicated complete consumption of starting material the reaction was quenched with H₂O. The organic phase was separated, and the aqueous phase was extracted with EtOAc. The combined organic phase was then washed with brine, dried over Na₂SO₄ and concentrated under reduced pressure. The crude residue was purified by column chromatography (SiO₂, MeOH in DCM, 0 to 10%) to give 4-cyclopropyl-7-oxo-1-(2-trimethylsilylethoxymethyl)-6H-pyrrolo[2,3-c]pyridine-2-carbaldehyde.

Step e: To the product of step d (3.32 g, 10 mmol, 1.0 equiv.) in dichloromethane (100 mL, 0.1 M) was added (S)-3-methylpiperidine hydrochloride (1.36 g, 10 mmol, 1.0 equiv.) and triethylamine (2.8 mL, 20 mmol, 2.0 equiv.). The resulting mixture was stirred at room temperature for 10 min before NaBH(OAc)₃ (3.18 g, 15 mmol, 1.5 equiv.) was added. The mixture was stirred at room temperature for 16 h. The reaction was quenched with aq. sat. NaHCO₃, the organic phase was separated, and the aqueous layer was extracted with EtOAc. The combined organic phase was dried over Na₂SO₄, concentrated and the crude residue was purified by column chromatography (SiO₂, MeOH in DCM, 0 to 10%) to afford 4-cyclopropyl-2-[[(3S)-3-methylpiperidin-1-yl]methyl]-1-(2-trimethylsilylethoxymethyl)-6H-pyrrolo[2,3-c]pyridin-7-one.

Step f: A mixture of methyl 2-bromo-5-cyanobenzoate (4.0 g, 16.66 mmol, 1.0 equiv.), (2,6-dichloropyridin-4-yl)boronic acid (3.84 g, 16.66 mmol, 1.0 equiv.) and K₂CO₃ (8.3 g, 49.98 mmol, 3.0 equiv.) in dioxane (40 mL) and H₂O (4 mL) was added Pd(dtBPF)Cl₂ (1.3 g, 1.7 mmol, 0.1 equiv.) at rt under nitrogen atmosphere. The resulting mixture was stirred for 3 h at 75° C. under nitrogen atmosphere. Once TLC analysis indicated complete conversion of methyl 2-bromo-5-cyanobenzoate the reaction was cooled to room temperature and quenched with sat. aq. NH₄Cl solution. The organic phase was separated, and the aqueous layer was extracted with EtOAc. The combined organic phase was dried over Na₂SO₄, concentrated and the crude residue was purified by column chromatography (SiO₂, hexane in EtOAc, 0 to 60%) to produce methyl 5-cyano-2-(2,6-dichloropyridin-4-yl)benzoate.

Step g: To the solution of methyl 5-cyano-2-(2,6-dichloropyridin-4-yl)benzoate (1.45 g, 4.723 mmol, 1.0 equiv.) in THF/H₂O (20 mL/5 mL, 0.2 M) LiOH (0.6 g, 23.6 mmol, 5.0 equiv.) was added. The resulting mixture was stirred at rt for 3 h. Once complete consumption of starting material was observed by LCMS analysis the reaction mixture was acidified to pH˜3 with aq. 4M HCl at 0° C. The mixture partitioned between water and EtOAc, the organic phase was separated, and the aqueous layer was extracted with EtOAc. The combined organic phase was dried over Na₂SO₄ and concentrated under reduced pressure to provide 5-cyano-2-(2,6-dichloropyridin-4-yl)benzoic acid.

Step h: To the mixture of product of step g (1.32 g, 4.5 mmol, 1.0 equiv.) and 1-amino-3-methylthiourea (500 mg, 4.5 mmol, 1.0 equiv.) in DMF (20 mL, 0.2 M) DIPEA (5.5 mL, 27 mmol, 6.0 equiv.) was added followed by T₃P (6.5 g, 18.5 mmol, 4.0 equiv., 50% in EtOAc). The resulting solution was stirred at rt for 3 h. Then the reaction was partitioned between water and EtOAc, the organic phase was separated, and the aqueous layer was extracted with EtOAc. The combined organic extract was washed with water, dried over Na₂SO₄ and concentrated to dryness under reduced pressure. The obtained crude product was purified by column chromatography (SiO₂, hexane in EtOAc, 0 to 100%) to afford 1-[[5-cyano-2-(2,6-dichloropyridin-4-yl)benzoyl]amino]-3-methylthiourea.

Step i: Thiourea product of step h (1.2 g, 3.158 mmol, 1.0 equiv.) was mixed with an aq. solution of sodium bicarbonate (100 mL, 1 M). The resulting mixture was heated at 80° C. and stirred for 1 h. The resulting mixture was allowed to cool to rt, diluted with water and carefully acidified with aq. 1M HCl to pH˜3. The product was extracted with EtOAc, then the combined organic phase was dried over Na₂SO₄, concentrated and the crude residue was purified by column chromatography (SiO₂, MeOH in DCM, 0 to 20%) to produce 4-(2,6-dichloropyridin-4-yl)-3-(4-methyl-5-sulfanyl-1,2,4-triazol-3-yl)benzonitrile.

Step j: To the product of step i (750 mg, 2.078 mmol, 1.0 equiv.) in dichloromethane (20 mL, 0.05 M) hydrogen peroxide (1.2 g, 10.4 mmol, 15 equiv., 30 wt %) and acetic acid (255 mg, 4.156 mmol, 2.0 equiv.) were added sequentially at 0° C. The reaction was allowed to warm to rt and stirred for 2 h. The resulting mixture was diluted with water, and the product was extracted with dichloromethane. The combined extract was washed with aq. sat. sodium bicarbonate, dried over Na₂SO₄ and concentrated to dryness under reduced pressure. The crude residue was fractionated by column chromatography (SiO₂, MeOH in DCM, 0 to 20%) to afford 4-(2,6-dichloropyridin-4-yl)-3-(4-methyl-1,2,4-triazol-3-yl)benzonitrile.

Step k: To a mixture of the product from step j (50 mg, 0.1205 mmol, 1.0 equiv.) and 4-cyclopropyl-2-[[(3S)-3-methylpiperidin-1-yl]methyl]-1-(2-trimethylsilylethoxymethyl)-6H-pyrrolo[2,3-c]pyridin-7-one (60 mg, 0.1807 mmol, 1.5 equiv.) in dioxane (3 mL, 0.04 M) was added CuI (24 mg, 0.1205 mmol, 1.0 equiv.), DMEDA (45 mg, 0.482 mmol, 4.0 equiv.) and K₂CO₃ (53 mg, 0.3615 mmol, 3.0 equiv.). The reaction was degassed by purging N₂ for 5 min and heated at 110° C. for 12 h under vigorous stirring. The reaction was allowed to cool to rt, diluted with water and EtOAc, the organic phase was separated, and the aqueous layer was additionally extracted with EtOAc. The combined organic phase was dried over Na₂SO₄, concentrated and the crude residue was purified by column chromatography (MeOH in DCM, 0 to 20%) to provide corresponding coupling product.

Step l: To a solution of the crude product from step k in dichloromethane (3 mL, 0.04 M) was added trifluoracetic acid (1 mL). The resulting solution was stirred at rt for 1 h. The solvent was removed under reduced pressure, then the dry residue was dissolved in 7M NH₃ in MeOH (3 mL), and the obtained solution was stirred for 30 min at rt. Upon concentration to dryness the residue was fractionated by reversed phase prep-HPLC to furnish the title compound. ¹H NMR (400 MHz, CDCl₃) δ 12.33 (s, 1H), 8.26 (s, 1H), 7.92 (dd, J=8.0, 1.7 Hz, 1H), 7.87 (d, J=1.6 Hz, 1H), 7.78 (d, J=1.3 Hz, 1H), 7.69 (d, J=8.0 Hz, 1H), 7.37 (d, J=1.3 Hz, 1H), 7.18 (d, J=1.2 Hz, 1H), 6.51 (d, J=2.1 Hz, 1H), 4.33 (d, J=14.1 Hz, 2H), 3.50 (s, 1H), 3.42 (s, 3H), 3.37 (s, 1H), 2.28 (s, 1H), 2.18-1.98 (m, 1H), 1.92 (d, J=13.4 Hz, 1H), 1.86-1.77 (m, 1H), 1.69 (s, 4H), 1.08 (d, J=13.1 Hz, 1H), 0.96 (d, J=6.5 Hz, 3H), 0.90-0.79 (m, 2H), 0.76-0.55 (m, 2H). ESI MS [M+H]⁺ for C₃₂H₃₂ClN₈O, calcd 580.1, found 580.1.

### Example 2: 4-[2-(2-{[(S)-3-methyl-1-piperidyl]methyl}-4-cyclopropyl-7-oxo-1,6-dihydro-1,6-diaza-6-indenyl)-6-cyclopropyl-4-pyridyl]-3-(4-methyl-4H-1,2,4-triazol-3-yl)benzonitrile

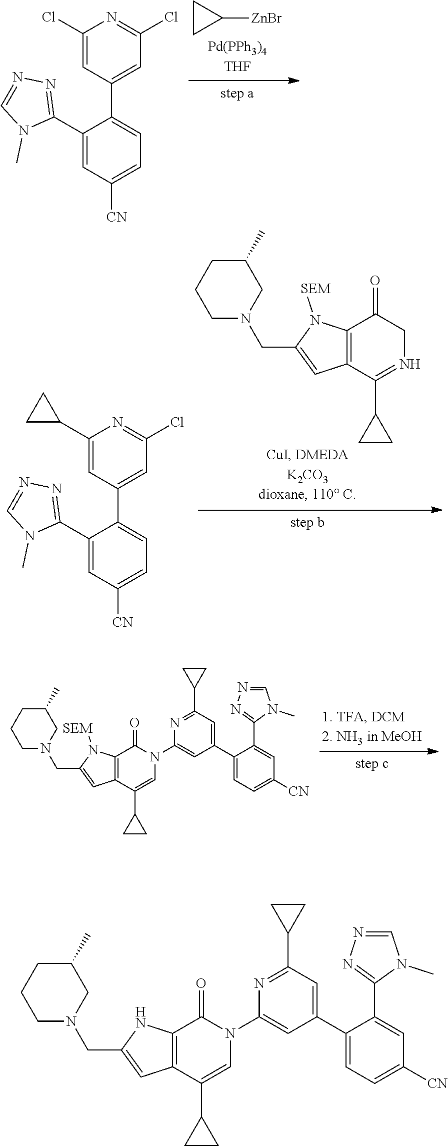

Step a: To a solution of 4-(2,6-dichloropyridin-4-yl)-3-(4-methyl-1,2,4-triazol-3-yl)benzonitrile (113 mg, 0.3424 mmol, 1.0 equiv., prepared according to example 1) in THE (5 mL, 0.07M) was added bromo(cyclopropyl)zinc (1.03 mL, 0.5136 mmol, 1.5 equiv., 0.5 M) and tetrakis(triphenylphosphine)palladium (80 mg, 0.06848 mmol, 0.2 equiv.) at rt under nitrogen atmosphere. The resulting mixture was stirred at rt for 2 h at 100° C. The mixture was allowed to cool down to rt and quenched with sat. aq. NH₄Cl solution, the organic phase was separated, and the aqueous layer was extracted with EtOAc. The combined organic phase was dried over Na₂SO₄, concentrated and the crude residue was purified by column chromatography (SiO₂, MeOH in DCM, 0 to 20%) to afford 4-(2-chloro-6-cyclopropylpyridin-4-yl)-3-(4-methyl-1,2,4-triazol-3-yl)benzonitrile.

Step b: To a solution of the product from step a (50 mg, 0.1190 mmol, 1.0 equiv.) and 4-cyclopropyl-2-[[(3S)-3-methylpiperidin-1-yl]methyl]-1-(2-trimethylsilylethoxymethyl)-6H-pyrrolo[2,3-c]pyridin-7-one (40 mg, 0.1190 mmol, 1.0 equiv., prepared according to example 1) in dioxane (3 mL, 0.04 M) was added CuI (23 mg, 0.12 mmol, 1.0 equiv.), DMEDA (42 mg, 0.476 mmol, 4.0 equiv.) and K₂CO₃ (50 mg, 0.3570 mmol, 3.0 equiv.). The resulting solution was stirred at 100° C. for 12 h. The reaction was quenched with H₂O, the organic phase was separated, and the aqueous layer was extracted with EtOAc. The combined organic phase was dried over Na₂SO₄, concentrated under reduced pressure, and the crude residue was purified by column chromatography (MeOH in DCM, 0 to 20%) to provide a mixture enriched with the desired coupling product. The obtained material was used for the next step without further purification.

Step c: To a solution of the crude product from step b in dichloromethane (3 mL, 0.04 M) was add trifluoroacetic acid (1 mL). The resulting solution was stirred at rt for 1 h. The solvent was removed under reduced pressure, and the obtained residue was redissolved in 7M NH₃ in MeOH (3 mL) and stirred for 30 min at rt. The solvent was evaporated, and the residue was purified by prep-HPLC to furnish the title compound. ¹H NMR (400 MHz, CDCl₃) δ 8.16 (s, 1H), 7.94 (d, J=1.6 Hz, 1H), 7.90 (dd, J=8.1, 1.7 Hz, 1H), 7.70 (d, J=8.0 Hz, 1H), 7.61 (d, J=1.4 Hz, 1H), 7.27 (d, J=1.4 Hz, 1H), 6.81 (d, J=1.5 Hz, 1H), 6.47 (s, 1H), 3.99 (s, 2H), 3.21 (s, 3H), 3.07 (m, 2H), 2.31 (m, 1H), 1.96 (tt, J=8.0, 4.9 Hz, 2H), 1.91-1.84 (m, 2H), 1.84-1.73 (m, 4H), 1.10-0.92 (m, 4H), 0.91-0.84 (m, 5H), 0.69-0.61 (m, 2H). ESI MS [M+H]⁺ for C₃₅H₃₇N₈O, calcd 585.7, found 585.1.

### Example 3: 4-(2-{N-methyl[2-(methylamino)ethyl]amino}-6-(2-{[(S)-3-methyl-1-piperidyl]methyl}-4-cyclopropyl-7-oxo-1,6-dihydro-1,6-diaza-6-indenyl)-4-pyridyl)-3-(4-methyl-4H-1,2,4-triazol-3-yl)benzonitrile

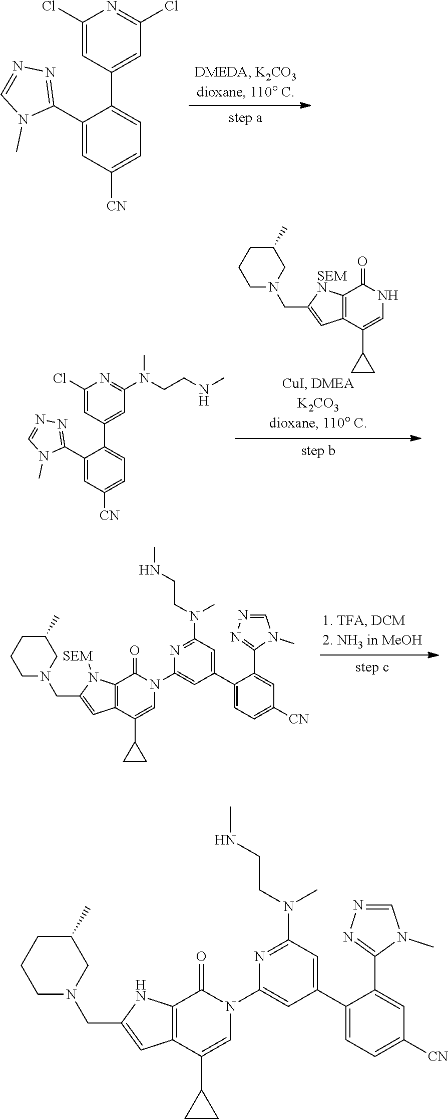 [SMILES: Cn1cnnc1-c1cc(C#N)ccc1-c1cc(Cl)nc(Cl)c1 ; CNCCN(C)c1cc(-c2ccc(C#N)cc2-c2nncn2C)cc(Cl)n1 ; C[C@H]1CCCN(Cc2cc3c(C4CC4)c[nH]c(=O)c3n2COCC[Si](C)(C)C)C1 ; CNCCN(C)c1cc(-c2ccc(C#N)cc2-c2nncn2C)cc(-n2cc(C3CC3)c3cc(CN4CCC[C@H](C)C4)n(COCC[Si](C)(C)C)c3c2=O)n1 ; CNCCN(C)c1cc(-c2ccc(C#N)cc2-c2nncn2C)cc(-n2cc(C3CC3)c3cc(CN4CCC[C@H](C)C4)[nH]c3c2=O)n1]

Step a: To a solution of 4-(2,6-dichloropyridin-4-yl)-3-(4-methyl-1,2,4-triazol-3-yl)benzonitrile (100 mg, 0.3030 mmol, 1.0 equiv., prepared according to example 1) in dioxane, (5 mL, 0.06M) potassium carbonate (210 mg, 1.5 mmol, 5.0 equiv.) and DMEDA (80 mg, 0.91 mmol, 3.0 equiv.) were added. The resulting mixture was heated at 110° C. for 12 h, then cooled to rt and partitioned between sat. aq. NH₄Cl solution and EtOAc. The organic phase was separated, and the aqueous layer was extracted with EtOAc. The combined organic phase was dried over Na₂SO₄, concentrated and the crude residue was purified by column chromatography (SiO₂, MeOH in DCM, 0 to 20%) to afford 4-[2-chloro-6-[methyl-[2-(methylamino)ethyl]amino]pyridin-4-yl]-3-(4-methyl-1,2,4-triazol-3-yl)benzonitrile.

Step b: To a solution of the product from step a (99 mg, 0.2598 mmol, 1.2 equiv.) and 4-cyclopropyl-2-[[(3S)-3-methylpiperidin-1-yl]methyl]-1-(2-trimethylsilylethoxymethyl)-6H-pyrrolo[2,3-c]pyridin-7-one (90 mg, 0.2165 mmol, 1.0 equiv., prepared according to example 1) in dioxane (3 mL, 0.04 M) was added CuI (41 mg, 0.2165 mmol, 1.0 equiv.), DMEDA (76 mg, 0.866 mmol, 4.0 equiv.) and K₂CO₃ (90 mg, 0.6495 mmol, 3.0 equiv.). The resulting mixture was stirred at 110° C. for 12 h under nitrogen atmosphere. The reaction was cooled to rt and partitioned between water and EtOAc, the organic phase was separated, and the aqueous layer was extracted with EtOAc. The combined organic phase was dried over Na₂SO₄, and the solvent was evaporated. The crude product was purified by column chromatography (SiO₂, MeOH in DCM, 0 to 20%) to give 4-[2-[4-cyclopropyl-2-[[(3S)-3-methylpiperidin-1-yl]methyl]-7-oxo-1-(2-trimethylsilyl-ethoxymethyl)pyrrolo[2,3-c]pyridin-6-yl]-6-[methyl-[2-(methylamino)ethyl]amino]pyridin-4-yl]-3-(4-methyl-1,2,4-triazol-3-yl)benzonitrile.

Step c: To a solution of the product from step b in dichloromethane (3 ml, 0.04 M) trifluoroacetic acid (1 mL) was added. The resulting solution was stirred at rt for 1 h. Then it was concentrated to dryness, and the residue was mixed with 7M NH₃ in MeOH (3 mL) and stirred for 30 min. Upon concentration the crude product was purified by prep-HPLC to furnish the title compound. ¹H NMR (400 MHz, CDCl₃) δ 8.57 (s, 1H), 8.47 (s, 1H), 8.14 (dd, J=8.0, 1.7 Hz, 1H), 8.10 (d, J=1.7 Hz, 1H), 7.96 (d, J=8.1 Hz, 1H), 6.81 (d, J=1.1 Hz, 1H), 6.68 (s, 1H), 6.62 (d, J=1.1 Hz, 1H), 6.55 (d, J=1.1 Hz, 1H), 4.05 (s, 2H), 3.87 (t, J=5.4 Hz, 2H), 3.42 (s, 2H), 3.23 (t, J=5.4 Hz, 2H), 3.18-3.07 (m, 3H), 3.05 (s, 3H), 2.74 (s, 3H), 2.41 (td, J=12.0, 3.0 Hz, 1H), 2.11 (t, J=11.3 Hz, 1H), 1.97 (dddd, J=13.5, 6.3, 5.2, 2.7 Hz, 1H), 1.87-1.65 (m, 5H), 1.10-1.00 (m, 1H), 1.00-0.84 (m, 5H), 0.76-0.62 (m, 2H). ESI MS [M+H]⁺ for C₃₆H₄₃N₁₀O, calcd 631.7, found 631.1.

### Example 4: 4-cyclopropyl-2-({[1-(hydroxymethyl)cyclobutyl]amino}methyl)-6-{3-[3-methyl-1-(4-methyl-4H-1,2,4-triazol-3-yl)cyclobutyl]phenyl}-1H,6H,7H-pyrrolo[2,3-c]pyridin-7-one

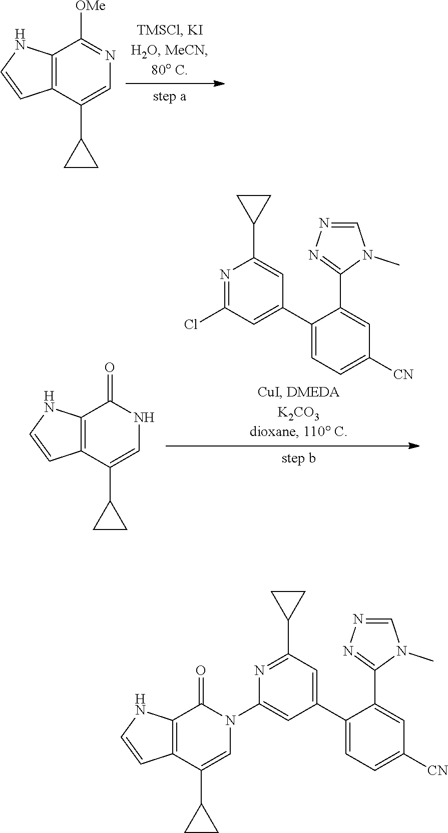 [SMILES: COc1ncc(C2CC2)c2cc[nH]c12 ; O=c1[nH]cc(C2CC2)c2cc[nH]c12 ; Cn1cnnc1-c1cc(C#N)ccc1-c1cc(Cl)nc(C2CC2)c1 ; Cn1cnnc1-c1cc(C#N)ccc1-c1cc(C2CC2)nc(-n2cc(C3CC3)c3cc[nH]c3c2=O)c1]

Step a: 4-Cyclopropyl-7-methoxy-1H-pyrrolo[2,3-c]pyridine (1.7 g, 8.9 mmol, 1 equiv., prepared according to example 1) was dissolved in acetonitrile (13 mL) and H₂O (13 mL). TMSCl (1.81 mL, 1.55 g, 14.3 mmol, 1.6 equiv.) and KI (2.28 g, 14.3 mmol, 1.6 equiv.) were added, and the reaction was heated to 80° C. Upon completion, the reaction was cooled to ambient temperature and partitioned between brine and dichloromethane. The organic layer was separated, dried over MgSO₄, and concentrated under reduced pressure. Purification by column chromatography (SiO₂, 0-30% EtOAc/DCM) yielded the desired 4-cyclopropyl-1,6-dihydropyrrolo[2,3-c]pyridin-7-one.

Step b: A solution of the product from step a (60 mg, 0.3448 mmol), 4-(2-chloro-6-cyclopropylpyridin-4-yl)-3-(4-methyl-1,2,4-triazol-3-yl)benzonitrile (116 mg, 0.3448 mmol, 1.0 equiv., prepared according to example 2) and K₂CO₃ (143 mg, 1.034 mmol, 3.0 equiv.) in dioxane (3 mL) was degassed with a stream of bubbling nitrogen for 10 min. Then CuI (65 mg, 0.3448 mmol, 1.0 equiv.) and DMEDA (121 mg, 1.379 mmol, 4.0 equiv.) were added, and the reaction was heated to 120° C. overnight in a sealed vial. On completion, the reaction was cooled to ambient temperature and poured into water. The resulting precipitate was collected and purified by normal phase column chromatography (SiO₂, 0-10% MeOH/DCM) followed by final purification by prep-HPLC to furnish the title compound. ¹H NMR (400 MHz, CDCl₃) δ 8.93 (s, 1H), 8.09 (s, 1H), 7.99 (d, J=1.7 Hz, 1H), 7.91 (dd, J=8.0, 1.7 Hz, 1H), 7.84 (d, J=3.1 Hz, 1H), 7.78 (d, J=8.1 Hz, 1H), 7.57 (d, J=1.4 Hz, 1H), 6.74 (d, J=3.1 Hz, 1H), 6.70 (d, J=3.6 Hz, 1H), 6.63 (d, J=1.4 Hz, 1H), 3.16 (s, 3H), 1.90 (ddt, J=9.3, 7.6, 4.7 Hz, 2H), 1.61 (s, 1H), 1.05-0.85 (m, 5H), 0.68-0.51 (m, 2H). ESI MS [M+H]⁺ for C₂₈H₂₄N₇O, calcd 474.5, found 474.1.

### Example 5: 2-{[(S)-3-Methyl-1-piperidyl]methyl}-4-cyclopropyl-6-{6-cyclopropyl-4-[4-methoxy-2-(4-methyl-4H-1,2,4-triazol-3-yl)phenyl]-2-pyridyl}-1,6-dihydro-1,6-diaza-7-indenone

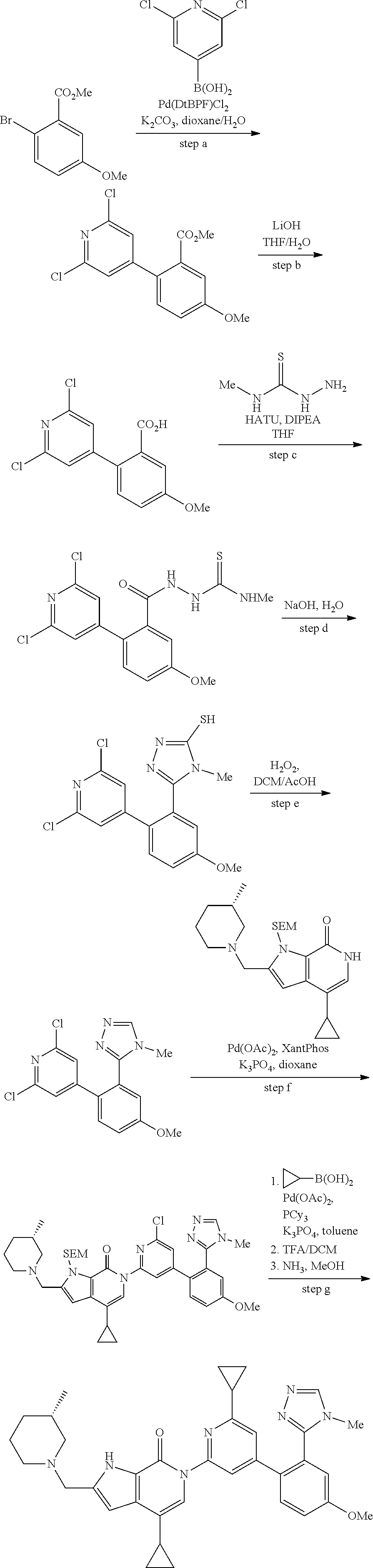

The title compound was prepared in a similar fashion to that described for Example 1. ¹H NMR (400 MHz, CDCl₃) δ 10.21 (s, 1H), 8.07 (s, 1H), 7.58 (d, J=1.5 Hz, 1H), 7.50 (d, J=8.5 Hz, 1H), 7.22-7.11 (m, 3H), 6.66 (d, J=1.4 Hz, 1H), 6.40 (d, J=1.7 Hz, 1H), 3.89 (s, 3H), 3.74 (s, 2H), 3.12 (s, 3H), 2.98-2.82 (m, 2H), 2.13-2.01 (m, 1H), 1.98-1.84 (m, 2H), 1.81-1.62 (m, 5H), 0.99-0.82 (m, 10H), 0.73-0.60 (m, 2H). ESI MS [M+H]⁺ for C₃₅H₃₉N₇O₂, calcd 590.3, found 590.3.

### Example 6: 2-{1[(S)-3-Methyl-1-piperidyl]methyl}-6-{6-chloro-4-[4-fluoro-2-(4-methyl-4H-1,2,4-triazol-3-yl)phenyl]-2-pyridyl}-4-cyclopropyl-1,6-dihydro-1,6-diaza-7-indenone

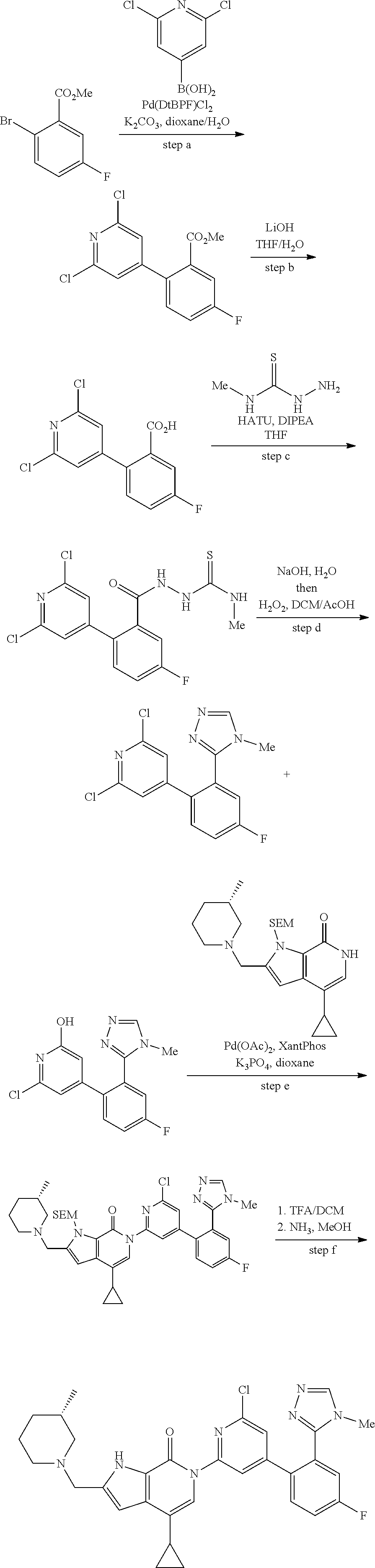

Step a: To a mixture of methyl 2-bromo-5-fluorobenzoate (4.66 g, 20 mmol, 1.0 equiv.) and (2,6-dichloropyridin-4-yl)boronic acid (4.60 g, 24 mmol, 1.2 equiv.) in dioxane/H₂O (4:1 v/v, 100 mL) was added Pd(DtBPF)Cl₂ (1.30 g, 2.0 mmol, 10 mol %) and K₂CO₃ (8.29 g, 60 mmol, 3.0 equiv.). The resulting mixture was heated at 60° C. under N₂ for 5 h before cooling to room temperature and diluting with EtOAc and water. The organic phase was separated, and the aqueous layer was extracted with EtOAc. The combined organic phase was dried over Na₂SO₄, concentrated, and the crude residue was purified by column chromatography (SiO₂, EtOAc in hexanes, 0 to 10%) to give the coupling product.

Step b: To the mixture of the product from step a (3.74 g, 12.5 mmol, 1.0 equiv.) in THF/H₂O (2:1 v/v, 60 mL) was added LiOH·H₂O (2.10 g, 50 mmol, 4.0 equiv.). The resulting mixture was heated at 60° C. for 2 h. After cooling to room temperature, the reaction mixture was neutralized with 1M HCl to pH˜5 and diluted with EtOAc. The organic phase was separated, and the aqueous layer was extracted with EtOAc. The combined organic phase was dried over Na₂SO₄, concentrated, providing the product (3.56 g, 99% yield) which was directly used in the next step.

Step c: To a solution of the product from step b (3.56 g, 12.4 mmol, 1.0 equiv.), 1-amino-3-methylthiourea (1.30 g, 12.4 mmol, 1.0 equiv.) and HATU (4.71 g, 12.4 mmol, 1.0 equiv.) in THE (60 mL) was added DIPEA (6.5 mL, 4.81 g, 37.2 mmol, 3.0 equiv.). The resulting mixture was stirred at room temperature for 2 h before quenched with H₂O. The mixture was then diluted with EtOAc. The organic phase was separated, and the aqueous layer was extracted with EtOAc twice. The combined organic phase was dried over Na₂SO₄, concentrated. The crude product was directly used in the next step without purification

Step d: To the crude product from step c (˜12.4 mmol) was added aq. 1M NaOH (40 mL). The resulting homogenous mixture was heated at 100° C. for 1.5 h. The reaction was carefully monitored by LCMS to avoid extensive S_{N}Ar substitution in 2,6-dichloropyridine fragment. Once completed the reaction mixture was cooled to rt, neutralized with 1M HCl to pH˜5 and diluted with EtOAc. The organic phase was separated, and the aqueous layer was extracted with EtOAc twice. The combined organic phase was dried over Na₂SO₄, and the solvent was evaporated under reduced pressure. The crude residue was then dissolved in dichloromethane (60 mL), followed by the addition of acetic acid (2.1 mL, 2.22 g, 37 mmol, 3.0 equiv.) and 30 wt % H₂O₂ aqueous solution (6.3 mL, 62 mmol, 5.0 equiv.) at 0° C. Upon complete addition of reagents, the cooling bath was removed, and the resulting mixture was stirred at room temperature for 1 h. Then it was quenched with aq. sat. NaHCO₃ to pH˜8. The resulting mixture was then extracted with dichloromethane/MeOH mixture (10:1 v/v) four times. The combined organic phase was dried over Na₂SO₄, concentrated, and the crude residue was purified by column chromatography (SiO₂, MeOH in DCM, 0 to 10%) to give 2,6-dichloro-4-[4-fluoro-2-(4-methyl-1,2,4-triazol-3-yl)phenyl]pyridine and 6-chloro-4-[4-fluoro-2-(4-methyl-1,2,4-triazol-3-yl)phenyl]pyridin-2-ol.

Step e: To a mixture of 2,6-dichloro-4-[4-fluoro-2-(4-methyl-1,2,4-triazol-3-yl)phenyl]pyridine (275 mg, 0.85 mmol, 1.0 equiv.), 4-cyclopropyl-2-[[(3S)-3-methylpiperidin-1-yl]methyl]-1-(2-trimethylsilylethoxymethyl)-6H-pyrrolo[2,3-c]pyridin-7-one (353 mg, 0.85 mmol, 1.0 equiv., obtained according to example 1) in dioxane (8.0 mL) was added Pd(OAc)₂ (19.1 mg, 0.085 mmol, 10 mol %), XantPhos (49.2 mg, 0.085 mmol, 10 mol %) and K₃PO₄ (541 mg, 2.6 mmol, 3.0 equiv.). The resulting mixture was heated at 100° C. for 2.5 h under nitrogen atmosphere. Once LCMS showed a complete conversion of coupling partners the reaction was cooled to room temperature and diluted with EtOAc. The organic extract was washed with water and brine, dried over Na₂SO₄ and concentrated. The crude residue was purified by prep-HPLC to afford 6-[6-chloro-4-[4-fluoro-2-(4-methyl-1,2,4-triazol-3-yl)phenyl]pyridin-2-yl]-4-cyclopropyl-2-[[(3S)-3-methylpiperidin-1-yl]methyl]-1-(2-trimethylsilylethoxymethyl)pyrrolo[2,3-c]pyridin-7-one.

Step f: The product from step e was treated with TFA/DCM (v/v 1:10, 2 mL) at room temperature overnight. The obtained solution was concentrated under reduced pressure, and the residue was redissolved in 7M NH₃ in methanol (2 mL). After stirring the reaction mixture for 1 h it was again concentrated, and the residue was directly fractionated by prep-HPLC to afford the title compound. ¹H NMR (400 MHz, CD₃OD) δ 8.48 (s, 1H), 7.82 (dd, J=8.7, 5.4 Hz, 1H), 7.58-7.48 (m, 3H), 7.30 (d, J=1.3 Hz, 1H), 7.13 (d, J=1.2 Hz, 1H), 6.64 (s, 1H), 3.98 (s, 2H), 3.40 (s, 3H), 3.18-3.02 (m, 2H), 2.36 (t, J=11.8 Hz, 1H), 2.06 (t, J=11.0 Hz, 1H), 1.94 (ttd, J=8.3, 5.2, 1.2 Hz, 1H), 1.87-1.57 (m, 4H), 1.13-0.85 (m, 6H), 0.74-0.60 (m, 2H). ESI MS [M+H]⁺ for C₃₁H₃₁ClFN₇O, calcd 572.2, found 572.2.

### Example 7: 2-{[(S)-3-methyl-1-piperidyl]methyl}-4-cyclopropyl-6-{4-[4-fluoro-2-(4-methyl-4H-1,2,4-triazol-3-yl)phenyl]-6-hydroxy-2-pyridyl}-1,6-dihydro-1,6-diaza-7-indenone

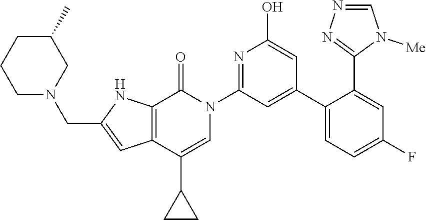 [SMILES: C[C@H]1CCCN(Cc2cc3c(C4CC4)cn(-c4cc(-c5ccc(F)cc5-c5nncn5C)cc(O)n4)c(=O)c3[nH]2)C1]

The title compound was prepared in a similar fashion to that described for example 6 from 6-chloro-4-[4-fluoro-2-(4-methyl-1,2,4-triazol-3-yl)phenyl]pyridin-2-ol. ¹H NMR (400 MHz, CDCl₃) δ 12.88 (s, 1H), 8.15 (s, 1H), 7.62 (dd, J=8.4, 5.3 Hz, 1H), 7.42-7.32 (m, 2H), 6.69 (s, 1H), 6.53 (s, 1H), 6.45 (s, 1H), 6.27 (s, 1H), 4.46 (d, J=14.4 Hz, 1H), 4.24 (d, J=14.4 Hz, 1H), 3.40 (d, J=11.1 Hz, 1H), 3.27 (d, J=9.6 Hz, 1H), 3.22 (s, 3H), 2.28 (t, J=11.9 Hz, 1H), 2.07-1.75 (m, 4H), 1.67 (d, J=12.0 Hz, 2H), 0.89 (dd, J=8.4, 1.7 Hz, 2H), 0.85-0.62 (m, 6H). ESI MS [M+H]⁺ for C₃₁H₃₂FN₇O₂, calcd 554.3, found 554.2.

### Example 8: 2-{[(S)-3-Methyl-1-piperidyl]methyl}-4-cyclopropyl-6-{4-[4-fluoro-2-(4-methyl-4H-1,2,4-triazol-3-yl)phenyl]-6-methoxy-2-pyridyl}-1,6-dihydro-1,6-diaza-7-indenone

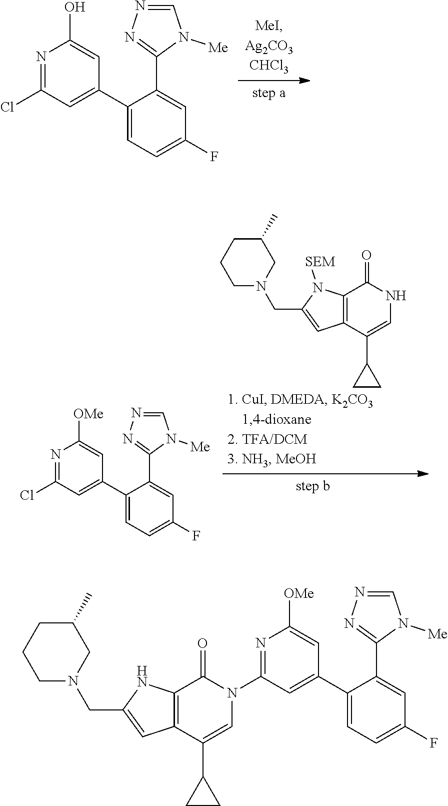 [SMILES: Cn1cnnc1-c1cc(F)ccc1-c1cc(O)nc(Cl)c1 ; C[C@H]1CCCN(Cc2cc3c(C4CC4)c[nH]c(=O)c3n2COCC[Si](C)(C)C)C1 ; COc1cc(-c2ccc(F)cc2-c2nncn2C)cc(Cl)n1 ; COc1cc(-c2ccc(F)cc2-c2nncn2C)cc(-n2cc(C3CC3)c3cc(CN4CCC[C@H](C)C4)[nH]c3c2=O)n1]

Step a: To a mixture of 6-chloro-4-[4-fluoro-2-(4-methyl-1,2,4-triazol-3-yl)phenyl]pyridin-2-ol (100 mg, 0.33 mmol, 1.0 equiv., see example 6 for detailed synthetic protocol) and Ag₂CO₃ (138 mg, 0.50 mmol, 1.5 equiv.) in CHCl₃ (3.0 mL) was added iodomethane (93.7 mg, 0.66 mmol, 2.0 equiv.). The resulting mixture was heated at 40° C. overnight before filtering through Celite® and concentrating under vacuum. The crude residue was purified by column chromatography (SiO₂, MeOH in DCM, 0 to 10%) to afford the corresponding alkylation product.

Step b: To a mixture of the product from step a (40.5 mg, 0.13 mmol, 1.0 equiv.), 4-cyclopropyl-2-[[(3S)-3-methylpiperidin-1-yl]methyl]-1-(2-trimethylsilylethoxymethyl)-6H-pyrrolo[2,3-c]pyridin-7-one (54.0 mg, 0.13 mmol, 1.0 equiv., prepared according to example 1) in dioxane (1.3 mL) was added CuI (24.8 mg, 0.13 mmol, 1.0 equiv.), N,N′-dimethylethylenediamine (22.9 mg, 0.26 mmol, 2.0 equiv.) and K₂CO₃ (52.5 mg, 0.38 mmol, 3.0 equiv.) The resulting mixture was heated at 100° C. overnight to give 60% conversion of coupling products according to LCMS analysis. The reaction mixture was cooled to room temperature, diluted with EtOAc, washed with H₂O and brine, filtered through Na₂SO₄, and concentrated. The residual material was then treated with TFA/DCM (v/v 1:10, 2 mL) at room temperature for 3 h. The mixture was concentrated under vacuum, then treated with 7M NH₃ in methanol (2 mL) for 30 min followed by the concentration. The crude residue was then purified by prep-HPLC to afford title compound. ¹H NMR (400 MHz, CDCl₃) δ 11.46 (s, 1H), 8.16 (s, 1H), 7.58 (dd, J=8.6, 5.4 Hz, 1H), 7.41-7.28 (m, 3H), 7.24 (d, J=1.3 Hz, 1H), 6.47 (s, 2H), 4.01 (s, 2H), 3.87 (s, 3H), 3.24 (s, 3H), 3.24-3.04 (m, 2H), 2.32 (t, J=11.6 Hz, 1H), 2.07-1.70 (m, 6jH), 1.04-0.78 (m, 6H), 0.65 (td, J=5.7, 4.1 Hz, 2H). ESI MS [M+H]⁺ for C₃₂H₃₄FN₇O₂, calcd 568.3, found 568.3.

### Example 9: 2-{[(S)-3-Methyl-1-piperidyl]methyl}-4-cyclopropyl-6-{6-ethoxy-4-[4-fluoro-2-(4-methyl-4H-1,2,4-triazol-3-yl)phenyl]-2-pyridyl}-1,6-dihydro-1,6-diaza-7-indenone

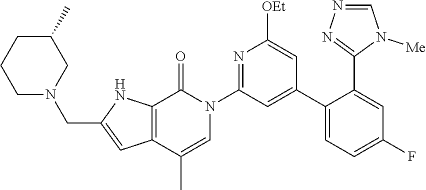 [SMILES: CCOc1cc(-c2ccc(F)cc2-c2nncn2C)cc(-n2cc(C)c3cc(CN4CCC[C@H](C)C4)[nH]c3c2=O)n1]

The title compound was prepared in a similar fashion to that described for example 8 from 6-chloro-4-[4-fluoro-2-(4-methyl-1,2,4-triazol-3-yl)phenyl]pyridin-2-ol (see example 6 for detailed synthetic protocol) and iodoethane. ¹H NMR (400 MHz, CDCl₃) δ 9.69 (s, 1H), 8.10 (s, 1H), 7.58 (dd, J=8.6, 5.4 Hz, 1H), 7.39 (dd, J=8.6, 2.7 Hz, 1H), 7.36-7.29 (m, 2H), 7.20 (d, J=1.2 Hz, 1H), 6.40 (d, J=1.2 Hz, 1H), 6.37 (s, 1H), 4.24 (q, J=7.0 Hz, 2H), 3.64 (s, 2H), 3.21 (s, 3H), 2.93-2.73 (m, 2H), 2.03-1.93 (m, 1H), 1.88 (ttd, J=8.4, 5.2, 1.2 Hz, 1H), 1.78-1.55 (m, 5H), 1.36 (t, J=7.0 Hz, 3H), 0.93-0.79 (m, 6H), 0.71-0.61 (m, 2H). ESI MS [M+H]⁺ for C₃₃H₃₆FN₇O₂, calcd 582.3, found 582.3.

### Example 10: 2-{[(S)-3-Methyl-1-piperidyl]methyl}-4-cyclopropyl-6-{6-cyclopropyl-4-[4-fluoro-2-(4-methyl-4H-1,2,4-triazol-3-yl)phenyl]-2-pyridyl}-1,6-dihydro-1,6-diaza-7-indenone

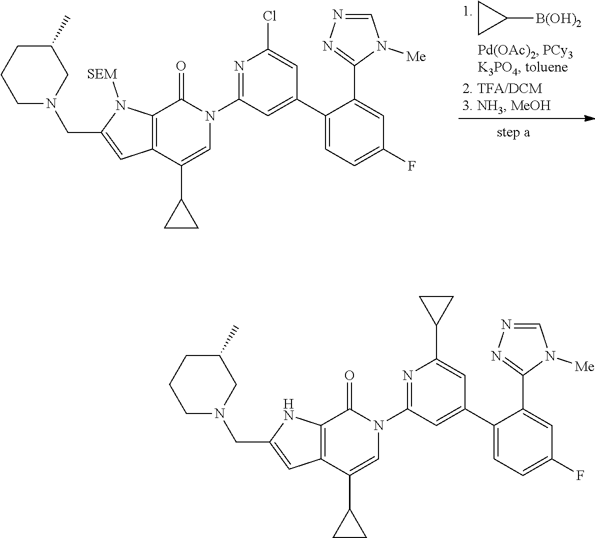 [SMILES: C[C@H]1CCCN(Cc2cc3c(C4CC4)cn(-c4cc(-c5ccc(F)cc5-c5nncn5C)cc(Cl)n4)c(=O)c3n2COCC[Si](C)(C)C)C1 ; OB(O)C1CC1 ; C[C@H]1CCCN(Cc2cc3c(C4CC4)cn(-c4cc(-c5ccc(F)cc5-c5nncn5C)cc(C5CC5)n4)c(=O)c3[nH]2)C1]

Step a: To a solution of 6-[6-chloro-4-[4-fluoro-2-(4-methyl-1,2,4-triazol-3-yl)phenyl]pyridin-2-yl]-4-cyclopropyl-2-[[(3S)-3-methylpiperidin-1-yl]methyl]-1-(2-trimethylsilylethoxymethyl)pyrrolo[2,3-c]pyridin-7-one (30.1 mg, 0.043 mmol, 1.0 equiv., prepared according to example 6) and cyclopropylboronic acid (17.2 mmol, 0.20 mmol, 4.7 equiv.) in toluene (0.50 mL) was added Pd(OAc)₂ (4.7 mg, 0.021 mmol, 50 mol %), PCy₃ (5.9 mg, 0.021 mmol, 50 mol %) and K₃PO₄ (42.5 mg, 0.20 mmol, 4.7 equiv.). The resulting mixture was heated at 100° C. under N₂ for 1 h. After cooling to room temperature, the reaction mixture was diluted with EtOAc, washed with H₂O and brine, filtered through Na₂SO₄, and concentrated. The residue was then treated with TFA/DCM (v/v 1:10, 2 mL) at room temperature for 3 h, and then concentrated. The obtained crude material was then treated with 7M NH₃ in methanol (2 mL) for 30 min, followed by concentration. The crude residue was then purified by HPLC to afford the title compound. ¹H NMR (400 MHz, CD₃OD) δ 8.46 (s, 1H), 7.80 (dd, J=8.7, 5.4 Hz, 1H), 7.53 (td, J=8.4, 2.7 Hz, 1H), 7.45 (dd, J=8.7, 2.7 Hz, 1H), 7.28 (d, J=1.4 Hz, 1H), 7.05 (d, J=1.2 Hz, 1H), 6.96 (d, J=1.4 Hz, 1H), 6.57 (s, 1H), 3.82 (s, 2H), 3.29 (s, 3H), 3.06-2.89 (m, 2H), 2.16 (t, J=11.6 Hz, 1H), 2.05 (tt, J=8.2, 4.8 Hz, 1H), 1.94 (dddd, J=11.0, 6.5, 5.3, 2.7 Hz, 1H), 1.86 (t, J=11.0 Hz, 1H), 1.80-1.56 (m, 4H), 1.05-0.85 (m, 1OH), 0.70-0.58 (m, 2H). ESI MS [M+H]⁺ for C₃₄H₃₆FN₇O, calcd 578.3, found 578.3.

### Example 11: 2-{[(S)-3-methyl-1-piperidyl]methyl}-4-cyclopropyl-6-{6-(ethylamino)-4-[4-fluoro-2-(4-methyl-4H-1,2,4-triazol-3-yl)phenyl]-2-pyridyl}-1,6-dihydro-1,6-diaza-7-indenone

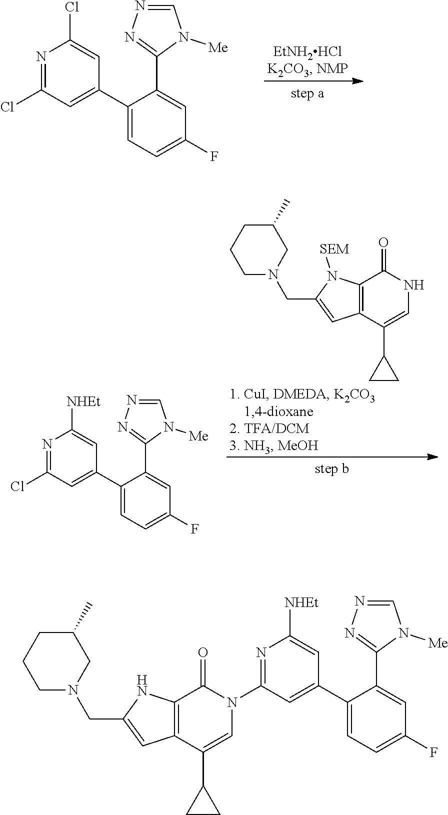

Step a: To a mixture of 2,6-dichloro-4-[4-fluoro-2-(4-methyl-1,2,4-triazol-3-yl)phenyl]pyridine (140 mg, 0.43 mmol, 1.0 equiv., obtained according to example 6) and K₂CO₃ (594 mg, 4.3 mmol, 10 equiv.) in NMP (2.0 mL) was added EtNH₂·HCl (351 mg, 4.3 mmol, 10 equiv.). The resulting mixture was heated at 100° C. overnight. After cooling to room temperature, the reaction mixture was directly purified by reverse phase column chromatography (C18, MeCN in H₂O, 10 to 70%, 0.1% formic acid) to give S_{N}Ar product.

Step b: The coupling reaction was performed in a similar fashion to that described for example 8 to afford the title compound. ¹H NMR (400 MHz, CDCl₃) δ 10.79 (s, 1H), 8.09 (s, 1H), 7.57 (dd, J=8.6, 5.4 Hz, 1H), 7.38 (dd, J=8.6, 2.7 Hz, 1H), 7.31 (td, J=8.3, 2.7 Hz, 1H), 7.16 (d, J=1.2 Hz, 1H), 7.06 (d, J=1.3 Hz, 1H), 6.43 (s, 1H), 5.94 (s, 1H), 4.62 (s, 1H), 3.90-3.78 (m, 2H), 3.21 (s, 3H), 3.15-2.92 (m, 4H), 2.13 (t, J=11.7 Hz, 1H), 1.94-1.64 (m, 6H), 1.17 (t, J=7.2 Hz, 3H), 0.99-0.80 (m, 6H), 0.72-0.62 (m, 2H). ESI MS [M+H]⁺ for C₃₃H₃₇FN₈O, calcd 581.3, found 581.3.

### Example 12: 2-{[(S)-3-Methyl-1-piperidyl]methyl}-4-cyclopropyl-6-{4-[4-fluoro-2-(4-methyl-4H-1,2,4-triazol-3-yl)phenyl]-2-pyridyl}-1,6-dihydro-1,6-diaza-7-indenone

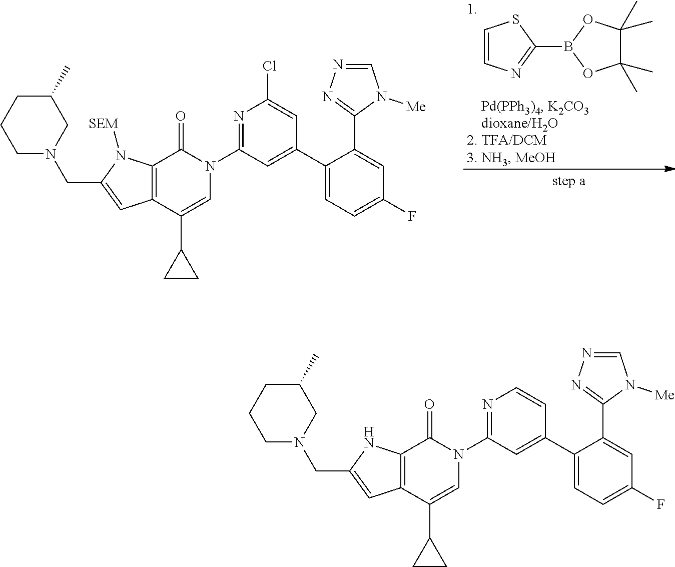 [SMILES: C[C@H]1CCCN(Cc2cc3c(C4CC4)cn(-c4cc(-c5ccc(F)cc5-c5nncn5C)cc(Cl)n4)c(=O)c3n2COCC[Si](C)(C)C)C1 ; CC1(C)OB(c2nccs2)OC1(C)C ; C[C@H]1CCCN(Cc2cc3c(C4CC4)cn(-c4cc(-c5ccc(F)cc5-c5nncn5C)ccn4)c(=O)c3[nH]2)C1]

Step a: To a mixture of 6-[6-chloro-4-[4-fluoro-2-(4-methyl-1,2,4-triazol-3-yl)phenyl]pyridin-2-yl]-4-cyclopropyl-2-[[(3S)-3-methylpiperidin-1-yl]methyl]-1-(2-trimethylsilylethoxymethyl)pyrrolo[2,3-c]pyridin-7-one (50.4 mg, 0.072 mmol, 1.0 equiv., prepared according to example 6) and 2-(4,4,5,5-tetramethyl-1,3,2-dioxaborolan-2-yl)-1,3-thiazole (16.7 mg, 0.079 mmol, 1.1 equiv.) in dioxane/H₂O (3:1 v/v, 1.0 mL) was added Pd(PPh₃)₄ (8.3 mg, 0.0072 mmol, 10 mol %) and K₂CO₃ (30.4 mg, 0.22 mmol, 3.0 equiv.). The resulting mixture was heated at 100° C. overnight. LCMS analysis showed no formation of the expected coupling product and exclusive formation of dechlorination product that was isolated using the following protocol. After cooling to room temperature, the reaction mixture was diluted with EtOAc, washed with H₂O and brine, filtered through Na₂SO₄, and concentrated. The residue was then treated with TFA/DCM (v/v 1:10, 2 mL) at room temperature for 3 h, and then concentrated. The crude material was then treated with 7M NH₃ in methanol (2 mL) for 30 min, followed by concentration. The obtained residue was directly purified by prep-HPLC to afford the title compound. ¹H NMR (400 MHz, CDCl₃) δ 10.17 (s, 1H), 8.39 (d, J=5.2 Hz, 1H), 8.08 (s, 1H), 7.93 (s, 1H), 7.59 (dd, J=8.6, 5.3 Hz, 1H), 7.41 (dd, J=8.5, 2.7 Hz, 1H), 7.36 (td, J=8.3, 2.7 Hz, 1H), 7.30 (d, J=1.2 Hz, 1H), 6.92 (dd, J=5.2, 1.6 Hz, 1H), 6.41 (s, 1H), 3.74 (s, 2H), 3.16 (s, 3H), 2.96-2.82 (m, 2H), 2.20-1.97 (m, 2H), 1.95-1.83 (m, 1H), 1.83-1.63 (m, 4H), 1.04-0.79 (m, 6H), 0.75-0.61 (m, 2H). ESI MS [M+H]⁺ for C₃₁H₃₂FN₇O, calcd 538.3, found 538.2.

### Example 13: 4-[2-(2-{[(S)-3-methyl-1-piperidyl]methyl}-4-cyclopropyl-7-oxo-1,6-dihydro-1,6-diaza-6-indenyl)-6-cyclopropyl-4-pyridyl]-3-(1-methyl-2-imidazolyl)benzonitrile

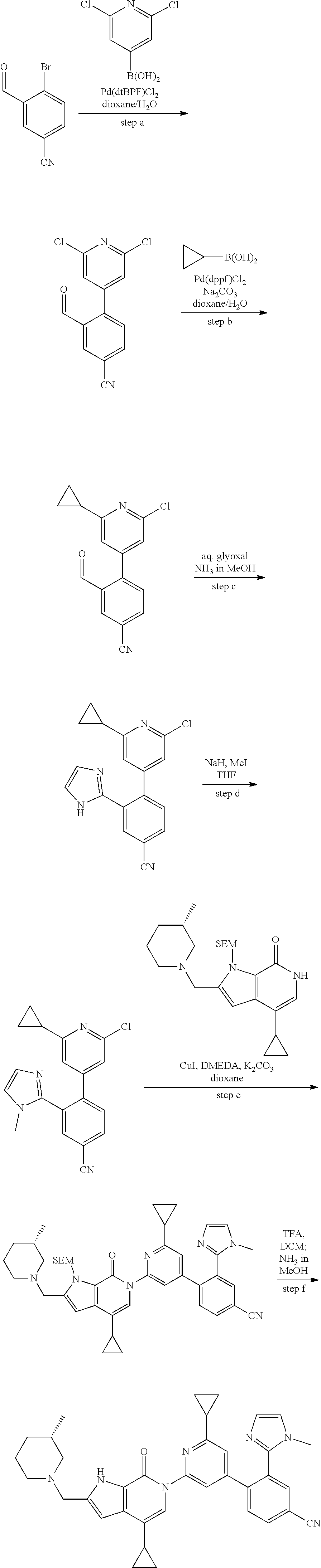

Step a: A mixture of 4-bromo-3-formylbenzonitrile (1.0 g, 5.0 mmol, 1.0 equiv.), (2,6-dichloropyridin-4-yl)boronic acid (960 mg, 5.0 mmol, 1.0 equiv.) and K₂CO₃ (2.1 g, 15.0 mmol, 3.0 equiv.) in dioxane/water (10 mL/2 mL, 0.2 M) was loaded in a 40 mL vial. Pd(dtBPF)Cl₂ (380 mg, 0.5 mmol, 0.1 equiv.) was added at rt under nitrogen atmosphere. The resulting mixture was stirred at rt for 1 h at 85° C. and cooled to rt. The mixture was partitioned between sat. aq. NH₄Cl and EtOAc. The organic phase was separated, and the aqueous layer was extracted with EtOAc. The combined organics was dried over Na₂SO₄, concentrated, and the crude residue was purified by column chromatography (SiO₂, hexane in EtOAc, 0 to 60%) to afford 4-(2,6-dichloropyridin-4-yl)-3-formylbenzonitrile.

Step b: To the product of step a (323 mg, 1.1661 mmol, 1.0 equiv.) in dioxane (5 mL, 0.2 M) was added Na₂CO₃ (1.75 mL, 3.5 mmol, 3.0 equiv., 2 N), cyclopropylboronic acid (110 mg, 1.3 mmol, 1.1 equiv.) and Pd(dppf)Cl₂ (85 mg, 0.1166 mmol, 0.1 equiv.) and the mixture was stirred at 90° C. for 3 h. The reaction was diluted with H₂O, the organic phase was separated, and the aqueous layer was extracted with EtOAc. The combined organic phase was dried over Na₂SO₄, concentrated and the crude residue was purified by column chromatography (SiO₂, hexane in EtOAc, 0 to 60%) to provide 4-(2-chloro-6-cyclopropylpyridin-4-yl)-3-formylbenzonitrile.

Step c: The product of step b (100 mg, 0.3521 mmol, 1.0 equiv.) was mixed with aq. glyoxal (0.45 mL, 2.5 mmol, 7.0 equiv., 40 wt % solution) and 7M NH₃ in MeOH (2.0 mL, 3.521 mmol, 10.0 equiv.). The obtained reaction mixture was stirred at rt for 72 h. The reaction was diluted with water and EtOAc, the organic phase was separated, and the aqueous layer was extracted with EtOAc. The combined organic extract was dried over Na₂SO₄ and concentrated. The crude residue was purified by column chromatography (SiO₂, MeOH in DCM, 0 to 10%) to afford 4-(2-chloro-6-cyclopropylpyridin-4-yl)-3-(1H-imidazol-2-yl)benzonitrile.

Step d: To a solution of the product of step c (84 mg, 0.2617 mmol, 1.0 equiv.) in THE (2.6 mL, 0.1 M) was added NaH (26 mg, 0.6542 mmol, 2.5 equiv., 60 wt % in mineral oil) at 0° C. The reaction mixture was stirred at 0° C. for 10 min before MeI (111 mg, 0.7851 mmol, 3.0 equiv.) was added. The mixture was stirred at rt for 3 h, then quenched with sat. aq. NH₄Cl solution and diluted with EtOAc. The organic phase was separated, and the aqueous layer was extracted with EtOAc. The combined organic phase was dried over Na₂SO₄, concentrated and the crude residue was purified by column chromatography (SiO₂, MeOH in DCM, 0 to 10%) to yield 4-(2-chloro-6-cyclopropylpyridin-4-yl)-3-(1-methylimidazol-2-yl)benzonitrile.

Step e: To a solution of the product from step d (36 mg, 0.1075 mmol, 1.2 equiv.) and 4-cyclopropyl-2-[[(3S)-3-methylpiperidin-1-yl]methyl]-1-(2-trimethylsilylethoxymethyl)-6H-pyrrolo[2,3-c]pyridin-7-one (40 mg, 0.08958 mmol, 1.0 equiv., obtained according to example 1) in dioxane (3 mL, 0.03 M) was added CuI (20 mg, 0.1075 mmol, 1.0 equiv.), DMEDA (38 mg, 0.43 mmol, 4.0 equiv.) and K₂CO₃ (45 mg, 0.3225 mmol, 3.0 equiv.). The resulting solution was stirred at 110° C. for 12 h in a sealed vial, then it was quenched with water and diluted with EtOAc. The organic phase was separated, and the aqueous layer was extracted with EtOAc. The combined organics was dried over Na₂SO₄ and concentrated to dryness. The crude residue was purified by column chromatography (MeOH in DCM, 0 to 20%) to afford 4-[2-cyclopropyl-6-[4-cyclopropyl-2-[[(3S)-3-methylpiperidin-1-yl]methyl]-7-oxo-1-(2-trimethylsilylethoxymethyl)pyrrolo[2,3-c]pyridin-6-yl]pyridin-4-yl]-3-(1-methylimidazol-2-yl)benzonitrile.

Step f: To a solution of the crude product from step e in dichloromethane (3 mL, 0.04 M) was added TFA (1 mL). The resulting solution was stirred at rt for 1 h, then solvent was evaporated, and the crude residue was dissolved in 7M NH₃ in MeOH (3 mL). After stirring for 30 min the mixture was concentrated to dryness, and the crude product was purified by prep-HPLC to furnish the title compound. ¹H NMR (400 MHz, CDCl₃) δ 7.95 (d, J=1.7 Hz, 1H), 7.82 (dd, J=8.0, 1.7 Hz, 1H), 7.77 (d, J=1.4 Hz, 1H), 7.67 (d, J=8.0 Hz, 1H), 7.29 (d, J=1.2 Hz, 1H), 7.15 (d, J=1.2 Hz, 1H), 6.84 (d, J=1.3 Hz, 1H), 6.50 (d, J=1.4 Hz, 1H), 6.41 (s, 1H), 3.76 (s, 2H), 3.08 (s, 3H), 2.90 (s, 2H), 2.08 (s, 1H), 1.91 (qd, J=7.9, 7.4, 3.7 Hz, 2H), 1.77 (m, 3H), 1.69 (s, 3H), 0.94 (dt, J=7.9, 3.2 Hz, 2H), 0.90-0.78 (m, 7H), 0.68 (td, J=5.8, 4.2 Hz, 2H). ESI MS [M+H]⁺ for C₃₆H₃₈N₇O, calcd 584.7, found 584.1.

### Example 14: 4-[2-(2-{[(S)-3-methyl-1-piperidyl]methyl}-4-cyclopropyl-7-oxo-1,6-dihydro-1,6-diaza-6-indenyl)-6-cyclopropyl-4-pyridyl]-3-{1-[(²H3)methyl]-2-imidazolyl}benzonitrile

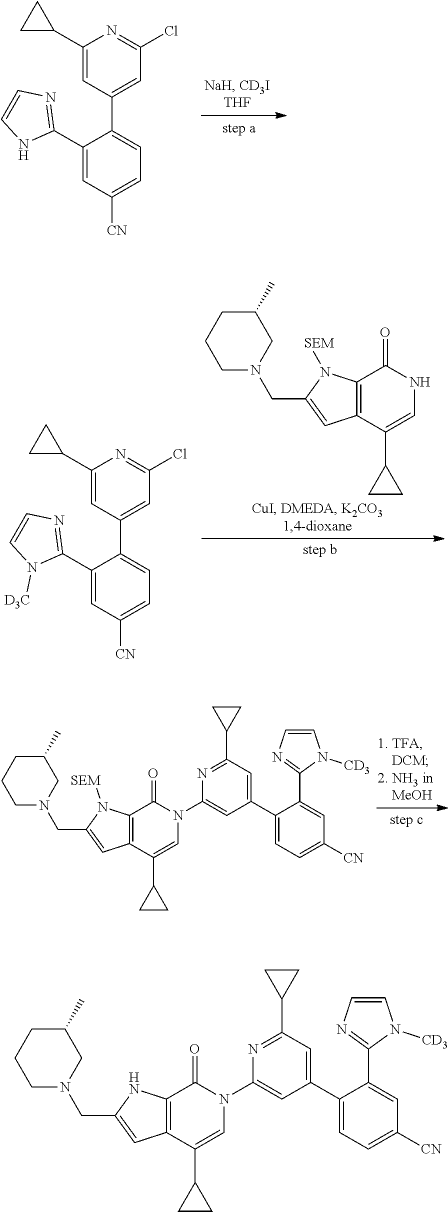 [SMILES: N#Cc1ccc(-c2cc(Cl)nc(C3CC3)c2)c(-c2ncc[nH]2)c1 ; [2H]C([2H])([2H])n1ccnc1-c1cc(C#N)ccc1-c1cc(Cl)nc(C2CC2)c1 ; C[C@H]1CCCN(Cc2cc3c(C4CC4)c[nH]c(=O)c3n2COCC[Si](C)(C)C)C1 ; [2H]C([2H])([2H])n1ccnc1-c1cc(C#N)ccc1-c1cc(C2CC2)nc(-n2cc(C3CC3)c3cc(CN4CCC[C@H](C)C4)n(COCC[Si](C)(C)C)c3c2=O)c1 ; [2H]C([2H])([2H])n1ccnc1-c1cc(C#N)ccc1-c1cc(C2CC2)nc(-n2cc(C3CC3)c3cc(CN4CCC[C@H](C)C4)[nH]c3c2=O)c1]

The title compound was prepared in a similar fashion to that described for Example 14 from CD₃I instead of CH₃I. ¹H NMR (400 MHz, CDCl₃) δ 10.32 (s, 1H), 7.95 (d, J=1.7 Hz, 1H), 7.82 (dd, J=8.1, 1.7 Hz, 1H), 7.79 (d, J=1.4 Hz, 1H), 7.67 (d, J=8.1 Hz, 1H), 7.29 (d, J=1.2 Hz, 1H), 7.16 (d, J=1.3 Hz, 1H), 6.84 (d, J=1.2 Hz, 1H), 6.49 (d, J=1.4 Hz, 1H), 6.40 (d, J=1.4 Hz, 1H), 3.74 (d, J=3.0 Hz, 2H), 2.90 (dd, J=25.5, 9.2 Hz, 2H), 2.07 (t, J=13.0 Hz, 1H), 1.98-1.84 (m, 2H), 1.76 (d, J=7.2 Hz, 3H), 1.69-1.58 (m, 3H), 1.04-0.91 (m, 2H), 0.90-0.81 (m, 7H), 0.68 (td, J=5.8, 4.2 Hz, 2H). ESI MS [M+H]⁺ for C₃₆H₃₅D₃N₇O, calcd 587.7, found 587.1.

### Example 15: 4-[2-(2-{1[(S)-3-methyl-1-piperidyl]methyl}-4-cyclopropyl-7-oxo-1,6-dihydro-1,6-diaza-6-indenyl)-6-cyclopropyl-4-pyridyl]-3-[1-(difluoromethyl)-2-imidazolyl]benzonitrile

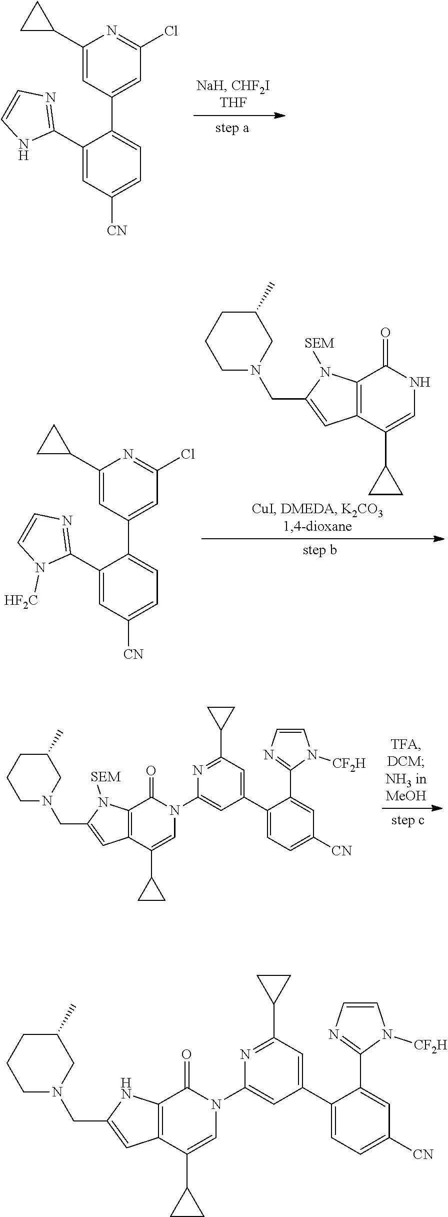

The title compound was prepared in a similar fashion to that described for Example 13 using CHF₂I instead of CH₃I. ¹H NMR (400 MHz, CDCl₃) δ 1H NMR (400 MHz, Chloroform-d) δ 12.11 (s, 1H), 7.88 (s, 1H), 7.81 (s, 1H), 7.72 (d, J=7.7 Hz, 1H), 7.32 (s, 1H), 7.25 (s, 1H), 7.21 (s, 1H), 6.63 (s, 1H), 6.44 (t, J=31 Hz, 1H), 4.28 (q, J=13.8 Hz, 2H), 3.48 (d, J=6.6 Hz, 1H), 3.34 (d, J=11.0 Hz, 1H), 2.51 (d, J=13.0 Hz, 1H), 2.29-2.01 (m, 4H), 1.95-1.81 (m, 4H), 1.67 (m, 2H), 1.10-0.99 (m, 1H), 0.99-0.84 (m, 8H), 0.66 (q, J=5.1 Hz, 2H). ESI MS [M+H]⁺ for C₃₆H₃₆F₂N₇O, calcd 620.7, found 620.1.

### Example 16: 4-[2-(2-{[(S)-3-methyl-1-piperidyl]methyl}-4-cyclopropyl-7-oxo-1,6-dihydro-1,6-diaza-6-indenyl)-6-(trifluoromethyl)-4-pyridyl]-3-(1-methyl-2-imidazolyl)benzonitrile

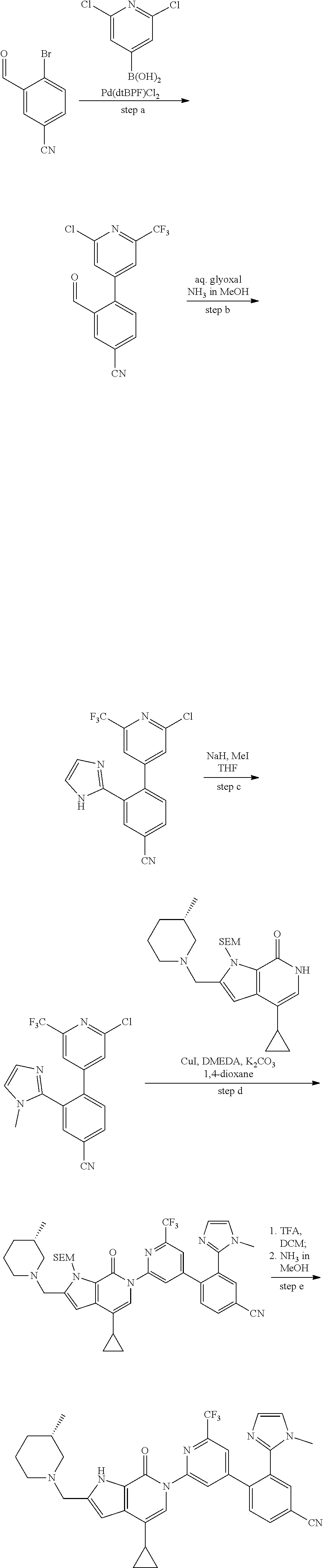

The title compound was prepared in a similar fashion to that described for Example 13 from [2-chloro-6-(trifluoromethyl)pyridin-4-yl]boronic acid. ¹H NMR (400 MHz, CDCl₃) δ 8.38 (s, 1H), 7.94 (s, 1H), 7.89 (d, J=8.1 Hz, 1H), 7.74 (d, J=8.0 Hz, 1H), 7.47 (d, J=1.3 Hz, 1H), 7.13 (s, 2H), 6.90 (s, 1H), 6.52 (s, 1H), 4.17 (s, 2H), 3.25 (s, 3H), 2.44 (s, 1H), 2.06 (s, 3H), 1.95-1.79 (m, 3H), 1.68 (m, 3H), 0.98-0.81 (m, 5H), 0.70 (dd, J=5.7, 4.1 Hz, 2H). ESI MS [M+H]⁺ for C₃₄H₃₃F₃N₇O, calcd 612.6, found 612.1.

### Example 17: 4-cyclopropyl-6-[3-[4-fluoro-2-(1-methylimidazol-2-yl)phenyl]phenyl]-2-[[(3S)-3-methylpiperidin-1-yl]methyl]-1H-pyrrolo[2,3-c]pyridin-7-one

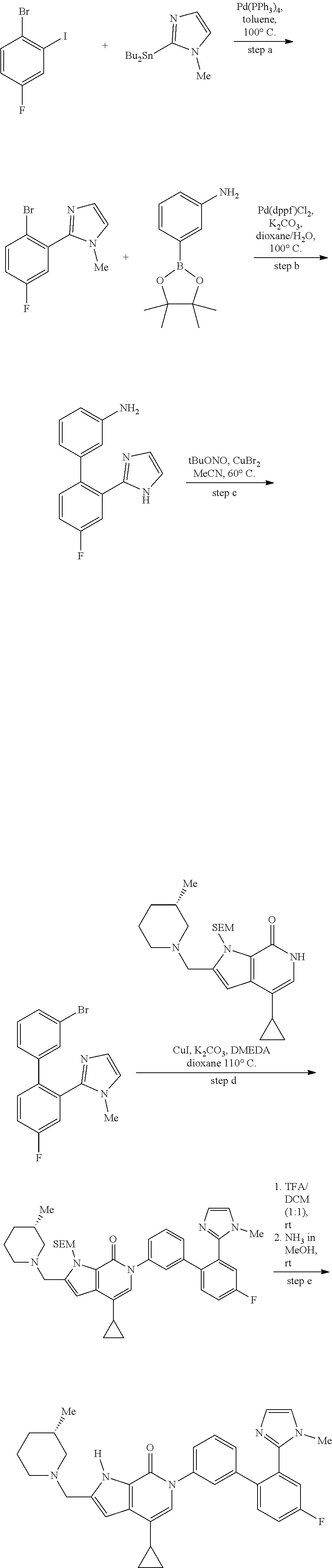

Step a: To a solution of 1-bromo-4-fluoro-2-iodobenzene (809 mg, 2.7 mmol, 1 equiv.) in toluene (13.5 mL, 0.2 M), was added tributyl-(1-methylimidazol-2-yl)stannane (1.0 g, 2.7 mmol. 1 equiv.) and the resulting solution was sparged with N₂ for 10 minutes. Then, Pd(PPh₃)₄ (310 mg, 0.27 mmol, 0.1 equiv.) was added, and the mixture was heated to 110° C. for 12 h. The reaction was cooled to ambient temperature, filtered over Celite®, and concentrated under vacuum. The crude residue was purified via silica gel flash column chromatography (0 to 20% MeOH/DCM) to afford 2-(2-bromo-5-fluorophenyl)-1-methylimidazole.

Step b: A 40 mL vial was charged with product from step a (400 mg, 1.58 mmol, 1 equiv.), 3-aminophenylboronic acid pinacol ester (414 mg, 1.9 mmol. 1.2 equiv.) and potassium carbonate (650 mg, 4.7 mmol, 3 equiv.). The reagents were suspended in the 3:1 mixture of dioxane/water (10 mL), and the resulting solution was sparged with N₂ for 10 minutes. Then Pd(dppf)Cl₂ (114 mg, 0.156 mmol, 0.1 equiv.) was added, and the mixture was heated to 95° C. for 6 hours. The reaction mixture was partitioned between EtOAc and water. The organic phase was separated, and the aqueous phase was additionally extracted with EtOAc. The combined organics were dried over Na₂SO₄ and concentrated to dryness. The crude residue was purified via silica gel flash column chromatography (0 to 100% EtOAc/hexane) to afford 3-[4-fluoro-2-(1-methylimidazol-2-yl)phenyl]aniline.

Step c: To a solution of the product from step b (220 mg, 0.82 mmol, 1.0 equiv.) and tert-butyl nitrite (375 mg, 3.28 mmol, 4.0 equiv.) in CH₃CN (4.1 mL, 0.2 M) was added CuBr₂ (219 mg, 0.98 mmol, 1.2 equiv.). The resulting mixture was stirred overnight at 60° C. Upon completion the reaction mixture was diluted with water and extracted with dichloromethane. The organic solution was washed with brine, dried over Na₂SO₄ and concentrated under vacuum. The crude residue was purified by column chromatography (EtOAc in hexanes, 0 to 50%) to give 2-[2-(3-bromophenyl)-5-fluorophenyl]-1-methylimidazole.

Step d: 4-cyclopropyl-6-[3-[4-fluoro-2-(1-methylimidazol-2-yl)phenyl]phenyl]-2-[[(3S)-3-methylpiperidin-1-yl]methyl]-1-(2-trimethylsilylethoxymethyl)pyrrolo[2,3-c]pyridin-7-one was prepared in a similar manner to Example 1) from 2-[2-(3-bromophenyl)-5-fluorophenyl]-1-methylimidazole and 4-cyclopropyl-2-[[(3S)-3-methylpiperidin-1-yl]methyl]-1,6-dihydropyrrolo[2,3-c]pyridin-7-one.

Step e: The reaction was performed in a similar fashion to Example 1) to afford the title compound. ¹H NMR (400 MHz, CDCl₃) δ 9.62 (s, 1H), 7.50 (dd, J=8.6, 5.6 Hz, 1H), 7.38-7.31 (m, 3H), 7.26-7.19 (m, 2H), 7.13 (d, J=1.2 Hz, 1H), 7.11-7.06 (m, 1H), 6.79 (d, J=1.2 Hz, 1H), 6.59 (d, J=1.2 Hz, 1H), 6.36 (d, J=2.0 Hz, 1H), 3.59 (d, J=2.6 Hz, 2H), 3.01 (s, 3H), 2.83-2.69 (m, 2H), 1.97-1.83 (m, 2H), 1.74-1.50 (m, 5H), 0.92-0.84 (m, 3H), 0.82 (d, J=5.7 Hz, 3H), 0.68-0.60 (m, 2H). ESI MS [M+H]⁺ for C₃₃H₃₄FN₅O, calcd 536.2, found 536.2.

### Example 18: 4-[2-cyclopropyl-6-[4-cyclopropyl-2-[[(1S,4S)-2,5-diazabicyclo[2.2.1]-heptan-2-yl]methyl]-7-oxo-1H-pyrrolo[2,3-c]pyridin-6-yl]pyridin-4-yl]-3-(4-methyl-1H-pyrazol-3-yl)benzonitrile

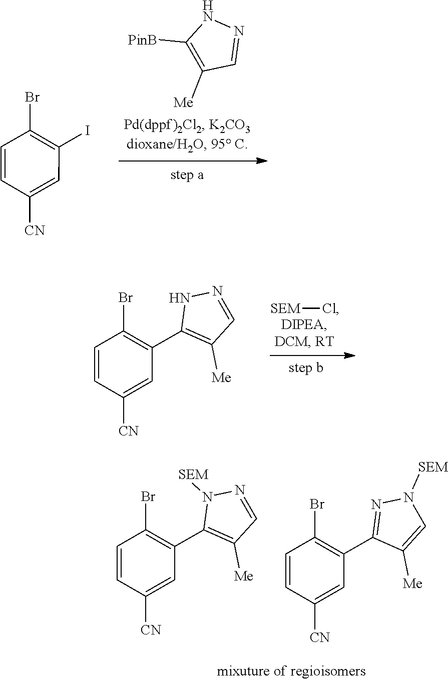

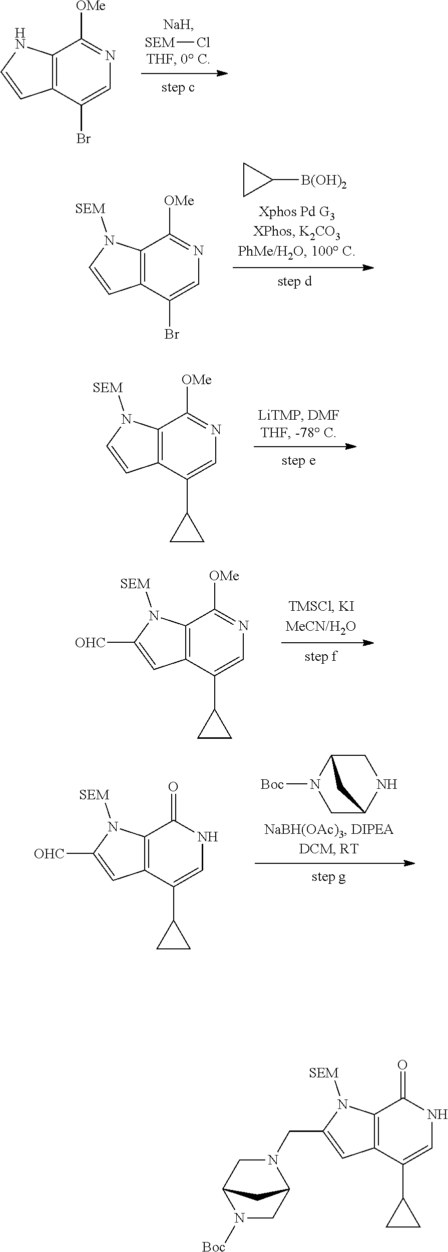

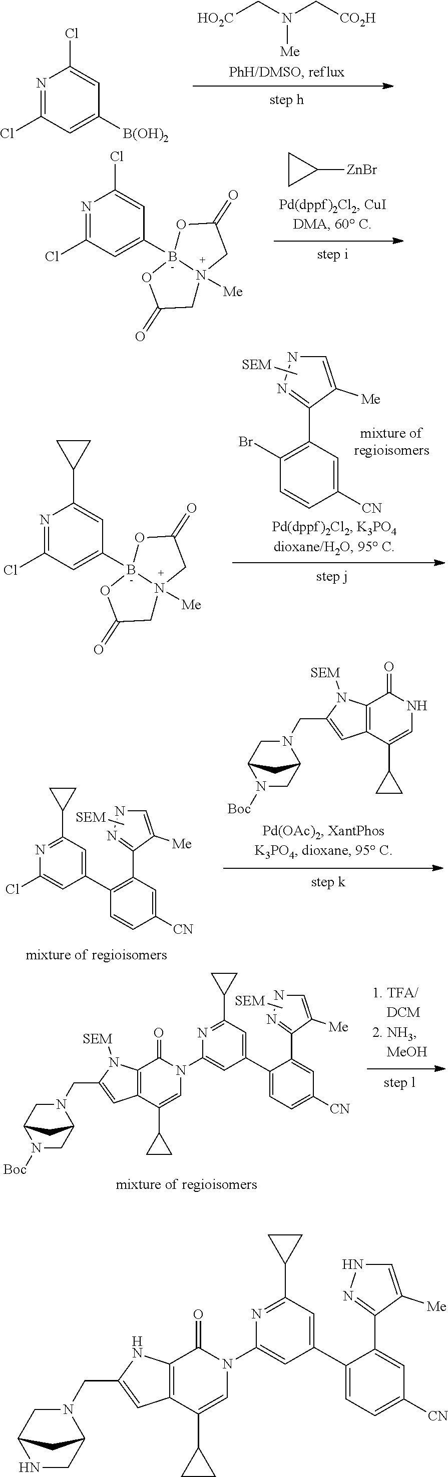

Step a: To a 100 mL round bottom flask was added 4-bromo-3-iodobenzonitrile (1.43 g, 4.64 mmol, 1.0 equiv.), 4-methyl-5-(4,4,5,5-tetramethyl-1,3,2-dioxaborolan-2-yl)-1H-pyrazole (970 mg, 4.64 mmol, 1.0 equiv.), and K₂CO₃ (1.92 g, 13.9 mmol, 3.0 equiv.). The reagents were dissolved in a 3:1 mixture of dioxane/water (30 mL) and the resulting mixture was sparged with N₂ for 10 minutes. Pd(dppf)₂Cl₂ (336 mg, 0.46 mmol, 0.1 equiv.) was added and the reaction mixture was heated to 95° C. and stirred for 1 hour at which point it was cooled to room temperature, quenched with a ˜1:1 mixture of saturated aqueous NaCl/water (150 mL) and extracted with EtOAc (2×75 mL). The combined organics were dried over Na₂SO₄, filtered, and concentrated under vacuum. The crude residue was purified via silica gel flash column chromatography (0 to 80% EtOAc/hexanes) to afford the desired product.

Step b: To a solution of the product from step a (713 mg, 2.72 mmol, 1.0 equiv.) in DCM (13 mL) was added DIPEA (0.72 mL, 4.1 mmol, 1.5 equiv.) followed by SEM-Cl (0.72 mL, 4.1 mmol, 1.5 equiv.). The reaction mixture was stirred at room temperature for 1 hour at which point it was quenched with water (100 mL) and extracted with DCM (2×50 mL). The combined organics were dried over Na₂SO₄, filtered, and concentrated under vacuum. The crude residue was purified via silica gel flash column chromatography (0 to 50% EtOAc/hexanes) to afford the desired product as a ˜1:1 mixture of regioisomers.

Step c: To a solution of 4-bromo-7-methoxy-1H-pyrrolo[2,3-c]pyridine (8.0, 35.24 mmol, 1.0 equiv.) in THE (100 ml, 0.3 M) was added NaH (2.54 g, 105.72 mmol, 3.0 equiv.) and SEM-Cl (6.46 g, 38.76 mmol, 1.1 equiv.) at 0° C. The resulting mixture was stirred at room temperature for 2 hours. The reaction mixture was quenched with H₂O, the organic phase was separated, and the aqueous layer was extracted with EtOAc. The combined organic phase was dried over Na₂SO₄, concentrated and the crude residue was purified by column chromatography (SiO₂, EtOAc in hexanes, 20 to 80%) to afford the desired product.

Step d: The product of step c (6.60 g, 18.44 mmol, 1.0 equiv.), cyclopropylboronic acid (2.0 g, 23.04 mmol, 1.25 equiv.) and K₂CO₃ (7.60 g, 55.31 mmol, 3.0 equiv.) were dissolved in a 5:1 mixture of toluene/H₂O (72 mL). The mixture was sparged for 2 mins with N₂ and Xphos Pd G3 (780 mg, 0.9218 mmol, 0.05 equiv.) and XPhos (703 mg, 1.4749 mmol, 0.08 equiv.) were added into the solution. The mixture was stirred at 90° C. for 12 h. After cooling down to 23° C., the reaction mixture was quenched with H₂O, the organic phase was separated, and the aqueous layer was extracted with EtOAc. The combined organic phase was dried over Na₂SO₄, concentrated and the crude residue was purified by column chromatography (SiO₂, EtOAc in hexanes, 20 to 80%) to afford the desired product.

Step e: To solution of 2,2,6,6-tetramethylpiperidine (1.53 ml, 8.98 mmol, 1.6 equiv.) in THE (50 mL, 0.18 M) was added n-butyllithium (2.5 M in hexanes, 3.6 ml, 8.98 mmol, 1.6 equiv.) slowly at −78° C. The mixture was stirred at −78° C. for 5 min followed by addition of the product of step d (1.79 g, 5.61 mmol, 1.0 equiv.) at −78° C. The resulting mixture was stirred at −78° C. for 1 h. Then, DMF (0.8 ml, 10.11 mmol, 1.8 equiv.) was added to the mixture and stirred for 30 min at −78° C. The reaction was quenched with sat. aq. NH₄Cl solution, the organic phase was separated, and the aqueous layer was extracted with EtOAc. The combined organic phase was dried over Na₂SO₄ and concentrated to dryness under reduced pressure. The crude residue was used in the next step without purification.

Step f: To a solution of the aldehyde from step e (1.95 g, 5.6 mmol, 1.0 equiv.) and KI (1.50 g, 8.96 mmol, 1.6 equiv.) in CH₃CN (50 mL, 0.1 M) was added TMSCl (977 mg, 8.96 mmol, 1.6 equiv.) dropwise at room temperature followed by water (0.1 ml). The resulting mixture was stirred at room temperature for 12 hours, then quenched with water. The organic phase was separated, and the aqueous phase was extracted with EtOAc. The combined organic phase was then washed with brine, dried over Na₂SO₄ and concentrated to dryness under reduced pressure. The crude residue was purified by column chromatography (SiO₂, MeOH in DCM, 0 to 10%) to afford the desired product.

Step g: To a solution of the product from step f (52 mg, 0.156 mmol, 1.0 equiv.) and tert-butyl (1S,4S)-2,5-diazabicyclo[2.2.1]heptane-2-carboxylate (62 mg, 0.313 mmol, 2.0 equiv.) in DCM (1.6 mL) was added DIPEA (70 μL, 0.390 mmol, 2.5 equiv.) followed by NaBH(OAc)₃ (87 mg, 0.390 mmol, 2.5 equiv.). The reaction mixture was stirred at room temperature for 1 h, at which point it was quenched with water (20 mL) and extracted with DCM (2×10 mL). The combined organics were dried over Na₂SO₄, filtered, and concentrated under vacuum. The crude residue was purified via silica gel flash column chromatography (0 to 20% MeOH/DCM) to afford the desired product.

Step h: A 2 L round bottom flask was charged with (2,6-dichloropyridin-4-yl)boronic acid (13.8 g, 72.2 mmol, 1.0 equiv.) and 2-[carboxymethyl(methyl)amino]acetic acid (10.6 g, 72.2 mmol, 1.0 equiv.). The reagents were dissolved in a 9:1 mixture of benzene/DMSO (720 mL), a Dean-Stark apparatus with reflux condenser was attached to the round bottom flask, and the reaction mixture was heated to reflux for 16 hours. The reaction mixture was cooled to room temperature and benzene was evaporated under vacuum. The remaining DMSO solution was poured into 600 mL of water. The resulting solid was collected by filtration and dried under vacuum to afford the desired product.

Step i: A solution of the product from step h (21.6 g, 71.3 mmol, 1.0 equiv.) in DMA (50 mL) was sparged with N₂ for 15 minutes at which point CuI (552 mg, 2.9 mmol, 0.04 equiv.) and Pd(dppf)₂Cl₂ (1.54 g, 2.1 mmol, 0.03 equiv.) were added. Bromo(cyclopropyl)zinc (0.5 M in THF, 107 mmol, 214 mL, 1.5 equiv.) was then added via cannula, and the reaction mixture was heated to 60° C. and stirred for 2 hours. The reaction mixture was cooled to room temperature and poured into saturated aqueous NH₄Cl (1 L) and extracted with EtOAc (2×350 mL). The combined organics were dried over Na₂SO₄, filtered, and concentrated under vacuum. The crude residue was triturated with DCM to induce crystallization of the desired product. The precipitate was collected by filtration and was dried on the filter to afford the desired product. ¹H NMR (400 MHz, DMSO-d₆) δ 7.32 (d, J=0.9 Hz, 1H), 7.19 (d, J=0.8 Hz, 1H), 4.37 (d, J=17.2 Hz, 2H), 4.18 (d, J=17.2 Hz, 2H), 2.61 (s, 3H), 2.11 (tt, J=8.1, 4.8 Hz, 1H), 1.02-0.95 (m, 2H), 0.93-0.86 (m, 2H). ESI MS [M+H]⁺ for C₁₃H₁₄BClN₂O₄, calcd 309.1, found 309.0.

Step j: To a 100 mL round bottom flask was added the product from step b (740 mg, 1.9 mmol, 1.0 equiv.), the product from step i (586 mg, 1.9 mmol, 1.0 equiv.), and K₃PO₄ (1.20 g, 5.7 mmol, 3.0 equiv.). The reagents were dissolved in a 4:1 mixture of dioxane/water (20 mL), and the resulting mixture was sparged with N₂ for 10 minutes. Pd(dppf)₂Cl₂ (146 mg, 0.20 mmol, 0.1 equiv.) was added and the reaction mixture was heated to 90° C. and stirred for 45 minutes at which point it was cooled to room temperature, quenched with a ˜1:1 mixture of saturated aqueous NaCl/water (150 mL), and extracted with EtOAc (2×50 mL). The combined organics were dried over Na₂SO₄, filtered, and concentrated under vacuum. The crude residue was purified via silica gel flash column chromatography (0 to 50% EtOAc/hexanes) followed by reverse phase column chromatography (C18 column, 0 to 100% MeCN/water) to afford the desired product as an inseparable ˜1:1 mixture of regioisomers.

Step k: A 40 mL vial was charged with the product from step g (76 mg, 0.15 mmol, 1.0 equiv.), product from step j (70 mg, 0.15 mmol, 1.0 equiv.), Pd(OAc)₂ (7.0 mg, 0.03 mmol, 0.2 equiv.), XantPhos (17.0 mg, 0.03 mmol, 0.2 equiv.) and K₃PO₄ (63 mg, 0.30 mmol, 2.0 equiv.). The reagents were dissolved in 1,4-dioxane, and the reaction mixture was sparged with N₂ for 10 minutes. The reaction was heated to 95° C. and stirred for 3 hours at which point it was quenched with a ˜1:1 mixture of saturated aqueous NaCl/water (20 mL) and extracted with EtOAc (2×10 mL). The combined organics were dried over Na₂SO₄, filtered, and concentrated under vacuum. The crude residue was purified via silica gel flash column chromatography (0 to 100% EtOAc/hexanes) to afford the desired product.

Step i: To a solution of the product from step k (45 mg, 0.05 mmol, 1.0 equiv.) in CH₂Cl₂ (1 mL) was added TFA (1 mL). The reaction mixture was stirred at room temperature for 2.5 h at which point it was diluted with toluene (5 mL) and concentrated under vacuum. The crude residue was dissolved in 7M NH₃ in MeOH (2 mL) and the reaction mixture was heated to 35° C. under stirring for 2 hours at which point it was directly concentrated under vacuum. The crude residue was purified via reverse phase prep-HPLC (C18 column, 5 to 70% MeCN/H₂O) to afford the desired product. ¹H NMR (400 MHz, CDCl₃) δ 7.78 (d, J=1.7 Hz, 1H), 7.71 (d, J=8.0 Hz, 1H), 7.66-7.53 (m, 2H), 7.41 (s, 1H), 7.15 (s, 1H), 6.67 (s, 1H), 6.36 (s, 1H), 3.71 (s, 2H), 3.42 (s, 1H), 2.92 (d, J=10.5 Hz, 1H), 2.81 (d, J=9.7 Hz, 1H), 2.64 (d, J=10.5 Hz, 1H), 2.43 (d, J=9.8 Hz, 1H), 1.99-1.81 (m, 2H), 1.91 (s, 3H), 1.00-0.93 (m, 2H), 0.90-0.82 (m, 4H), 0.68-0.61 (m, 2H). ESI MS [M+H]⁺ for C₃₅H₃₄N₈O, calcd 583.3, found 583.2.

### Example 19: 4-[2-cyclopropyl-6-[4-cyclopropyl-2-[[(3S)-3-methylpiperidin-1-yl]methyl]-7-oxo-1H-pyrrolo[2,3-c]pyridin-6-yl]pyridin-4-yl]-3-(4-methyl-1H-pyrazol-3-yl)benzonitrile

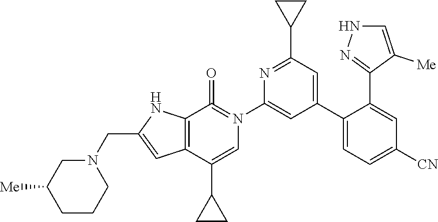

The title compound was prepared in a similar fashion to that described for Example 19 from 4-cyclopropyl-2-[[(3S)-3-methylpiperidin-1-yl]methyl]-1-(2-trimethylsilylethoxy-methyl)-6H-pyrrolo[2,3-c]pyridin-7-one and 4-(2-chloro-6-cyclopropylpyridin-4-yl)-3-[4-methyl-1-(2-trimethylsilylethoxymethyl)pyrazol-3-yl]benzonitrile. ¹H NMR (400 MHz, CDCl₃) δ 7.79 (d, J=1.6 Hz, 1H), 7.75 (dd, J=8.0, 1.7 Hz, 1H), 7.72 (s, 1H), 7.61 (d, J=8.0 Hz, 1H), 7.43 (s, 1H), 7.21 (d, J=1.2 Hz, 1H), 6.64 (s, 1H), 6.32 (s, 1H), 3.58 (s, 2H), 2.73 (dd, J=22.2, 10.6 Hz, 2H), 1.98-1.83 (m, 6H), 1.65-1.50 (m, 4H), 0.99-0.92 (m, 1H), 0.90-0.81 (m, 4H), 0.75 (d, J=6.2 Hz, 3H), 0.70-0.65 (m, 2H). ESI MS [M+H]⁺ for C₃₆H₃₇N₇O, calcd 584.3, found 584.2.

### Example 20: 4-[2-cyclopropyl-6-[4-cyclopropyl-2-[[(1-hydroxycyclobutyl)methylamino]-methyl]-7-oxo-1H-pyrrolo[2,3-c]pyridin-6-yl]pyridin-4-yl]-3-(4-methyl-1H-pyrazol-3-yl)benzonitrile

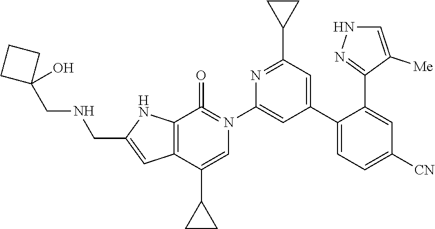

The title compound was prepared in a similar fashion to that described for Example 23 from 4-cyclopropyl-2-[[(1-hydroxycyclobutyl)methylamino]methyl]-1-(2-trimethylsilyl-ethoxymethyl)-6H-pyrrolo[2,3-c]pyridin-7-one and 4-(2-chloro-6-cyclopropylpyridin-4-yl)-3-[4-methyl-1-(2-trimethylsilylethoxymethyl)pyrazol-3-yl]benzonitrile. ¹H NMR (400 MHz, CDCl₃) δ 11.83 (s, 1H), 7.75 (s, 1H), 7.51 (dd, J=8.3, 1.6 Hz, 1H), 7.43-7.38 (m, 2H), 7.12 (d, J=8.2 Hz, 1H), 6.96 (s, 1H), 6.12 (d, J=1.4 Hz, 1H), 5.62 (d, J=2.0 Hz, 1H), 3.47 (s, 2H), 3.10-2.92 (m, 1H), 2.10 (s, 3H), 1.89-1.81 (m, 1H), 1.72-1.41 (m, 3H), 1.05-0.74 (m, 7H). ESI MS [M+H]⁺ for C₃₅H₃₅N₇O₂ calcd 586.3, found 586.2.

### Example 21: 4-cyclopropyl-6-[3-[4-fluoro-2-(4-methyl-1H-pyrazol-3-yl)phenyl]phenyl]-2-[[(3S)-3-methylpiperidin-1-yl]methyl]-1H-pyrrolo[2,3-c]pyridin-7-one

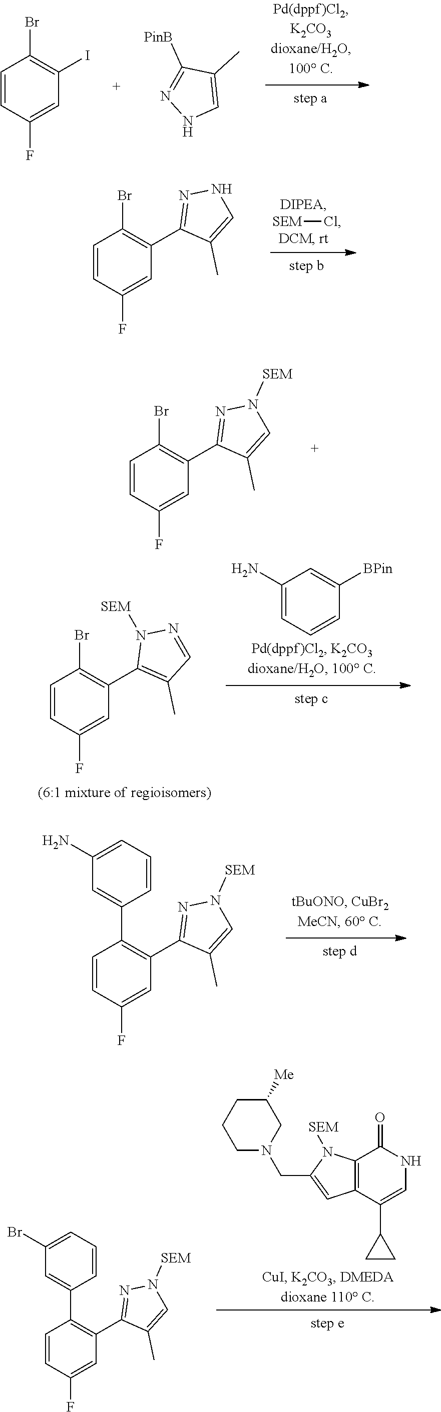

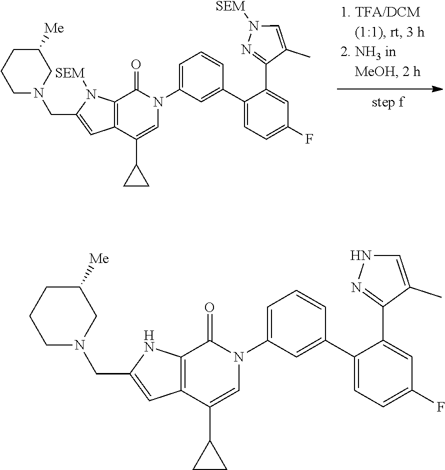 [SMILES: Cc1cn(COCC[Si](C)(C)C)nc1-c1cc(F)ccc1-c1cccc(-n2cc(C3CC3)c3cc(CN4CCC[C@H](C)C4)n(COCC[Si](C)(C)C)c3c2=O)c1 ; Cc1c[nH]nc1-c1cc(F)ccc1-c1cccc(-n2cc(C3CC3)c3cc(CN4CCC[C@H](C)C4)[nH]c3c2=O)c1]

Step a: To a 100 mL round bottom flask was added 1-bromo-4-fluoro-2-iodobenzene (3.0 g, 10 mmol. 1 equiv.), 4-methyl-5-(4,4,5,5-tetramethyl-1,3,2-dioxaborolan-2-yl)-1H-pyrazole (2.08 g, 10 mmol, 1.0 equiv.), and K₂CO₃ (4.4 g, 30 mmol, 3.0 equiv.). The reagents were dissolved in a 3:1 mixture of dioxane/water (50 mL), and the resulting solution was sparged with N₂ for 10 minutes. Pd(dppf)₂Cl₂ (731 mg, 1 mmol, 0.1 equiv.) was added, and the reaction mixture was heated to 95° C. and stirred for 6 hours at which point it was cooled to room temperature, quenched with water, and extracted with EtOAc (2×100 mL). The combined organics were dried over Na₂SO₄, filtered, and concentrated under vacuum. The crude residue was purified via silica gel flash column chromatography (0 to 50% EtOAc/hexanes) to afford the 3-(2-bromo-5-fluorophenyl)-4-methyl-1H-pyrazole.

Step b: To a solution of the product from step a (1.6 g, 6.27 mmol, 1.0 equiv.) in DCM (31 mL) was added DIPEA (1.63 mL, 9.41 mmol, 1.5 equiv.) followed by SEM-Cl (1.66 mL, 9.41 mmol, 1.5 equiv.). The reaction mixture was stirred at room temperature for 1 hour at which point it was quenched with water (100 mL) and extracted with DCM (2×50 mL). The combined organics were dried over Na₂SO₄, filtered, and concentrated under a vacuum. The crude residue was purified via silica gel flash column chromatography (0 to 80% EtOAc/hexanes) to afford the desired product as a ˜6:1 mixture of regioisomers.

Step c: A 40 mL vial was charged with a mixture of products from step b (335 mg, 0.87 mmol, 1 equiv.), 3-aminophenylboronic acid pinacol ester (231 mg, 1.05 mmol. 1.2 equiv.), K₂CO₃ (361 mg, 2.61 mmol, 3 equiv.). The reagents were suspended in the 3:1 mixture of dioxane/water (6 mL), and the resulting solution was sparged with N₂ for 10 minutes. Then, Pd(dppf)Cl₂ (64 mg, 0.087 mmol, 0.1 equiv.) was added, and the mixture was heated to 95° C. for 6 hours. The reaction mixture was partitioned between EtOAc and water, the organic layer was separated, and the aqueous phase was additionally extracted with EtOAc. The combined organics were dried over Na₂SO₄ and concentrated to dryness. The crude residue was purified via silica gel flash column chromatography (0 to 50% EtOAc/hexane) to afford 3-[4-fluoro-2-[4-methyl-1-(2-trimethylsilylethoxymethyl)pyrazol-3-yl]phenyl]aniline.

Step d: To the solution of product from step c (320 mg, 0.80 mmol, 1.0 equiv.) in CH₃CN (4.1 mL, 0.2 M) tert-butyl nitrite (370 mg, 3.22 mmol, 4.0 equiv.) and CuBr₂ (215 mg, 0.98 mmol, 1.2 equiv.) were sequentially added. The reaction mixture was stirred overnight at 60° C. Upon completion, the mixture was allowed to cool to rt, diluted with water and extracted with DCM. The combined organic extract was washed with brine, dried over Na₂SO₄, and concentrated under a vacuum. The crude residue was purified by column chromatography (EtOAc in hexanes, 0 to 50%) to afford 2-[[3-[2-(3-bromophenyl)-5-fluorophenyl]-4-methylpyrazol-1-yl]methoxy]ethyl-trimethylsilane.

Step e: The reaction was performed in a similar fashion to example 1, step f.

Step f: The reaction was performed in a similar fashion to example 1, step g to afford the title product. ¹H NMR (400 MHz, CDCl₃) δ 7.53-7.36 (m, 3H), 7.30 (t, J=7.8 Hz, 1H), 7.25-7.13 (m, 3H), 7.01 (d, J=7.7 Hz, 1H), 6.63 (s, 1H), 6.30 (d, J=1.9 Hz, 1H), 3.64 (s, 2H), 2.81-2.66 (m, 2H), 2.04-1.82 (m, 5H), 1.61-1.19 (m, 6H), 0.92-0.80 (m, 2H), 0.69 (d, J=6.5 Hz, 3H), 0.63 (q, J=5.1 Hz, 2H). ESI MS [M+H]⁺ for C₃₃H₃₄FN₅O, calcd 536.2, found 536.2.

### Example 22: 4-[2-cyclopropyl-6-[6-[[(3S)-3-methylpiperidin-1-yl]methyl]-4-oxo-5H-pyrrolo[3,2-d]pyrimidin-3-yl]pyridin-4-yl]-3-(4-methyl-1H-pyrazol-3-yl)benzonitrile

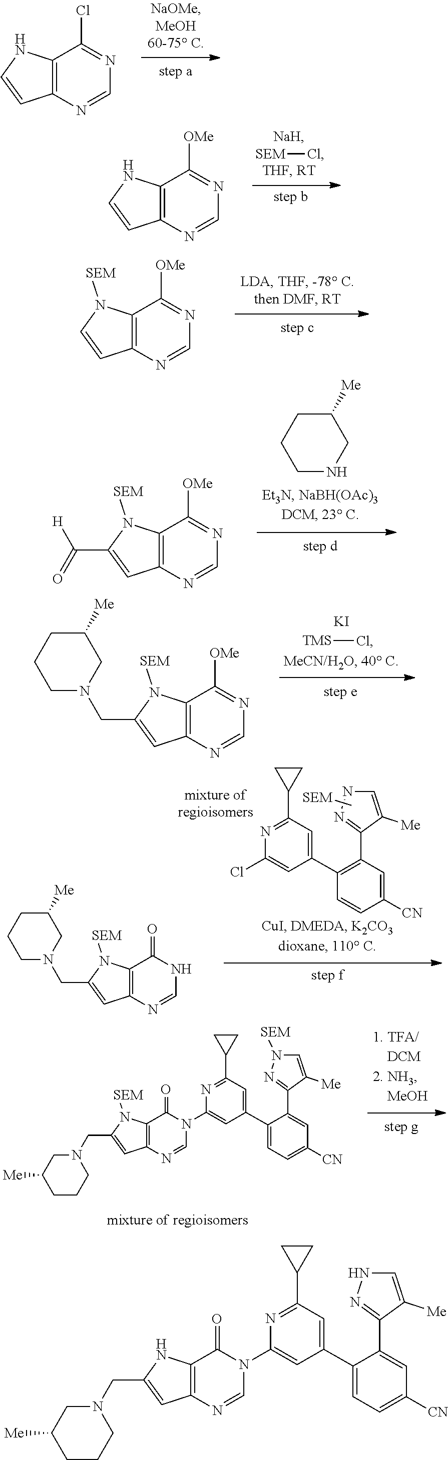

Step a: To a solution of 4-chloro-5H-pyrrolo[3,2-d]pyrimidine (4.0 g, 26 mmol, 1.0 equiv.) in MeOH was added NaOMe (2.1 g, 39 mmol, 1.5 equiv) at room temperature, and the mixture was stirred at 65° C. for 12 hours. An additional portion of NaOMe (2.8 g, 52 mmol, 2.0 equiv.) was added and the reaction mixture stirred at 65° C. for an additional 36 hours. After cooling to room temperature, the reaction mixture was quenched with H₂O, the organic phase was separated, and the aqueous layer was extracted with EtOAc. The combined organic phase was dried over Na₂SO₄, concentrated under vacuum, and the crude residue was purified by column chromatography (SiO₂, MeOH in DCM, 0 to 10%) to give desired product.

Step b: To a solution of the product from step a (3.4 g, 22.8 mmol, 1.0 equiv.) in THE (112.5 mL) was added NaH (1.8 g, 45.6 mmol, 2.0 equiv., 60% in mineral oil) at 0° C. After 15 minutes, SEM-Cl (5.7 g, 34.2 mmol, 1.5 equiv.) was added, and the reaction was allowed to warm to rt, and stirred for 12 hours. The reaction mixture was quenched with water, and the aqueous layer was extracted with EtOAc. The combined organic phase was dried over Na₂SO₄, concentrated under vacuum, and the crude residue was purified by column chromatography (SiO₂, EtOAc in hexanes, 10 to 70%) to give the desired product.

Step c: To a solution of the product from step b (2.4 g, 8.6 mmol, 1.0 equiv.) in THE (28 mL) at −78° C. was added LDA (5.16 mL, 10.32 mmol, 1.2 equiv, 2.0 M solution). The mixture was stirred for 30 minutes at −78° C., then DMF (0.94 g, 12.9 mmol, 1.5 equiv.) was added. The mixture was stirred at −78° C. for 15 min at which point the reaction mixture was warmed to room temperature and stirred for 2 hours. The reaction mixture quenched with saturated aqueous NH₄Cl and extracted with EtOAc. The combined organic phase was washed with brine, dried over Na₂SO₄ and concentrated. The crude residue was purified by column chromatography (SiO₂, EtOAc in hexanes, 0 to 60%) to give the desired product.

Step d: To a solution of the product of step c (1.53 g, 5.0 mmol, 1.0 equiv.) in DCM (5 mL) was added (S)-3-methylpiperidine hydrochloride (0.68 g, 5.0 mmol, 1.0 equiv.) and Et₃N (1.4 mL, 10 mmol, 2.0 equiv.) and the mixture was stirred at rt for 10 min. NaBH(OAc)₃ (2.11 g, 10 mmol, 2.0 equiv.) was added, and the mixture was stirred at room temperature for 12 hours. The reaction was quenched with saturated aqueous NaHCO₃ and extracted with EtOAc. The combined organic phase was dried over Na₂SO₄, concentrated to dryness under reduced pressure, and the crude residue was purified by column chromatography (SiO₂, MeOH in DCM, 0 to 10%) to give desired product.

Step e: To a solution of the product from step d (0.9 g, 2.3 mmol, 1.0 equiv.) in acetonitrile (20 mL) and water (0.125 mL, 6.90 mmol, 3.0 equiv.) KI (611 mg, 3.68 mmol, 1.6 equiv.) was added, followed by TMSCl (0.47 mL, 3.68 mmol, 1.6 equiv.). The reaction mixture was stirred for 6 hours at 45° C. After cooling the mixture to room temperature, it was quenched with H₂O and extracted with EtOAc. The combined organic phase was washed with brine, dried over Na₂SO₄ and concentrated under reduced pressure to dryness. The crude product was purified by column chromatography (SiO₂, MeOH in DCM, 0 to 20%) to give desired pyrimidinone intermediate.

Step f: To a solution of pyrimidinone intermediate from step e (45 mg, 0.12 mmol, 1.0 equiv.), 4-(2-chloro-6-cyclopropylpyridin-4-yl)-3-[4-methyl-1-(2-trimethylsilylethoxy-methyl)pyrazol-3-yl]benzonitrile (57 mg, 0.12 mmol, 1.0 equiv., obtained according to protocol described for example 19), and K₂CO₃ (51 mg, 0.37 mmol, 3.0 equiv.) in dioxane (3 mL) was added CuI (23 mg, 0.12 mmol, 1.0 equiv.) and DMEDA (26 μL, 0.24 mmol, 2.0 equiv.). The reaction mixture was heated to 100° C. and stirred for 16 hours at which point it was quenched with a ˜1:1 mixture of saturated aqueous NaCl/water (20 mL) and extracted with EtOAc (2×10 mL). The combined organics were dried over Na₂SO₄, filtered, and concentrated to dryness under reduced pressure. The crude residue was purified via silica gel flash column chromatography (0 to 100% EtOAc/hexanes) to afford the desired product.

Step g: To a solution of the product from step f (45 mg, 0.05 mmol, 1.0 equiv.) in DCM (1 mL) was added TFA (1 mL). The reaction mixture was stirred at 35° C. for 2.5 hours at which point it was diluted with toluene (5 mL) and concentrated under reduced pressure. The crude residue was dissolved in 7M NH₃ in MeOH (2 mL), and the reaction mixture was heated to 35° C. and stirred for 2 hours at which point it was directly concentrated under vacuum. The crude residue was purified via reverse phase prep-HPLC (C18 column, 5 to 70% MeCN/H₂O) to afford the desired product. ¹H NMR (400 MHz, CDCl₃) δ 11.43 (s, 1H), 7.84 (d, J=1.7 Hz, 1H), 7.76 (dd, J=8.0, 1.7 Hz, 1H), 7.59 (d, J=8.0 Hz, 1H), 7.45 (s, 1H), 7.40 (s, 1H), 6.95 (s, 1H), 6.35 (s, 1H), 3.54 (s, 2H), 2.73-2.62 (m, 2H), 1.99-1.91 (m, 1H), 1.84 (s, 4H), 1.65-1.35 (m, 4H), 1.01-0.94 (m, 4H), 0.74 (d, J=6.2 Hz, 3H). ESI MS [M+H]⁺ for C₃₂H₃₂N₈O, calcd 545.3, found 545.2.

### Example 23: 2-{[(S)-3-methyl-1-piperidyl]methyl}-6-{6-chloro-4-[4-fluoro-2-(1-methyl-5-imidazolyl)phenyl]-2-pyridyl}-4-cyclopropyl-1,6-dihydro-1,6-diaza-7-indenone

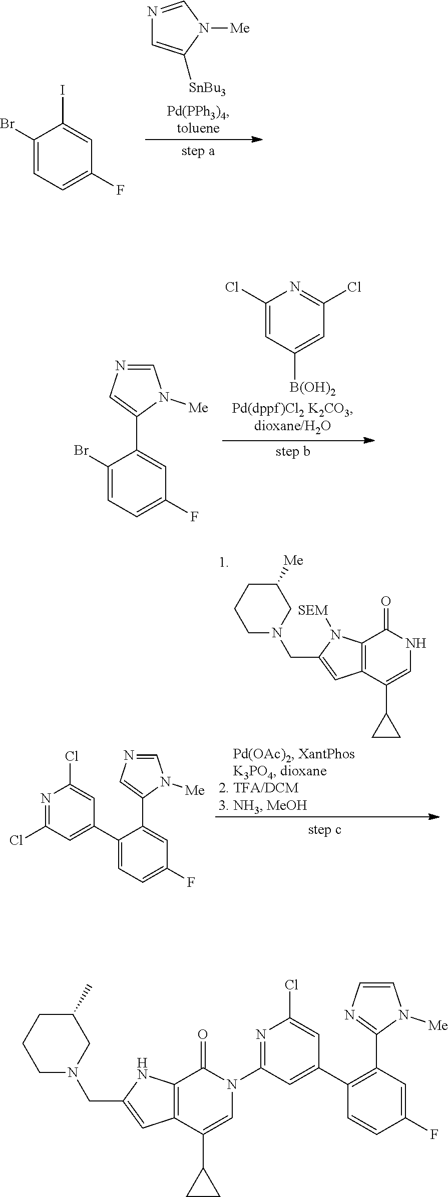

Step a: To a solution of 1-bromo-4-fluoro-2-iodobenzene (812 mg, 2.7 mmol, 1.0 equiv.) and tributyl-(3-methylimidazol-4-yl)stannane (1.00 g, 2.7 mmol, 1.0 equiv.) in toluene (20 mL) was added Pd(PPh₃)₄ (312 mg, 0.27 mmol, 10 mol %). The resulting mixture was heated at 110° C. for 1 h and cooled to room temperature. The solvent was removed under vacuum, and the residue was purified by column chromatography (SiO₂, MeOH in DCM, 0 to 10%) to yield the coupling product.

Step b: To a mixture of the product from step a (306 mg, 1.2 mmol, 1.0 equiv.) and (2,6-dichloropyridin-4-yl)boronic acid (230 mg, 1.2 mmol, 1.0 equiv.) in dioxane/H₂O (4:1 v/v, 15 mL) was added Pd(dppf)Cl₂ (87.8 mg, 0.12 mmol, 10 mol %) and K₂CO₃ (332 mg, 2.4 mmol, 2.0 equiv.). The reaction mixture was heated at 100° C. under N₂ for 5 h before cooling to room temperature. The organic phase was separated, and the aqueous layer was extracted with EtOAc. The combined organic phase was dried over Na₂SO₄, concentrated, and the crude residue was purified by reverse phase column chromatography (C18, MeCN in H₂O, 10 to 50%, 0.1% formic acid) to yield the corresponding triaryl compound.

Step c: The reaction was performed in a similar fashion to that described for Example 10. ¹H NMR (400 MHz, CDCl₃) δ 10.50 (s, 1H), 7.87 (d, J=1.3 Hz, 1H), 7.53 (dd, J=8.6, 5.5 Hz, 1H), 7.45 (s, 1H), 7.31 (d, J=1.3 Hz, 1H), 7.27-7.12 (m, 2H), 7.01 (d, J=1.1 Hz, 1H), 6.95 (d, J=1.3 Hz, 1H), 6.42 (s, 1H), 3.82 (s, 2H), 3.23 (s, 3H), 3.09-2.88 (m, 2H), 2.21-2.10 (m, 2H), 1.92-1.56 (m, 5H), 0.99-0.82 (m, 6H), 0.79-0.57 (m, 2H). ESI MS [M+H]⁺ for C₃₂H₃₂ClFN₆O, calcd 571.2, found 571.1.

### Example 24: 2-{[(S)-3-Methyl-1-piperidyl]methyl}-4-cyclopropyl-6-{6-cyclopropyl-4-[4-fluoro-2-(1-methyl-5-imidazolyl)phenyl]-2-pyridyl}-1,6-dihydro-1,6-diaza-7-indenone

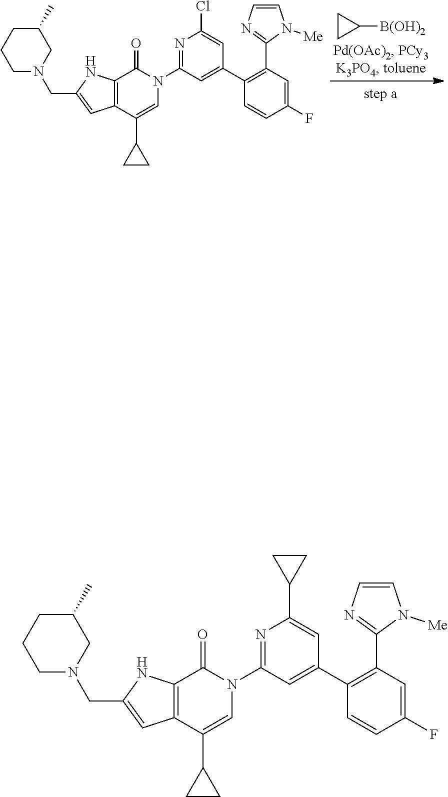

Step a: To a solution of 6-[6-chloro-4-[4-fluoro-2-(3-methylimidazol-4-yl)phenyl]pyridin-2-yl]-4-cyclopropyl-2-[[(3S)-3-methylpiperidin-1-yl]methyl]-1H-pyrrolo[2,3-c]pyridin-7-one (47.0 mg, 0.082 mmol, 1.0 equiv., prepared according to Example 24) and cyclopropylboronic acid (35.2 mmol, 0.41 mmol, 5.0 equiv.) in toluene (1.0 mL) was added Pd(OAc)₂ (9.2 mg, 0.041 mmol, 50 mol %), PCy₃ (11.5 mg, 0.041 mmol, 50 mol %) and K₃PO₄ (52.2 mg, 0.25 mmol, 3.0 equiv.). The resulting mixture was heated at 100° C. under N₂ for 1.5 h. Once LCMS analysis indicated full consumption of starting material the reaction was cooled to room temperature, diluted with water and EtOAc, and filtered through Celite® pad. The organic phase was separated, and the aqueous layer was additionally extracted with EtOAc. Organic extracts were combined, dried over Na₂SO₄, and concentrated to dryness under reduced pressure. The crude residue was purified by prep-HPLC to afford the title compound. ¹H NMR (400 MHz, CDCl₃) δ 10.02 (s, 1H), 7.64 (d, J=1.4 Hz, 1H), 7.53 (dd, J=8.6, 5.6 Hz, 1H), 7.40 (d, J=1.1 Hz, 1H), 7.21 (td, J=8.3, 2.7 Hz, 1H), 7.15 (dd, J=9.0, 2.7 Hz, 1H), 7.07 (d, J=1.1 Hz, 1H), 6.65 (d, J=1.4 Hz, 1H), 6.38 (s, 1H), 3.70 (d, J=3.5 Hz, 2H), 3.13 (s, 3H), 2.94-2.80 (m, 2H), 2.10-1.96 (m, 2H), 1.96-1.84 (m, 1H), 1.80-1.58 (m, 5H), 0.98-0.82 (m, 10H), 0.74-0.63 (m, 2H). ESI MS [M+H]⁺ for C₃₅H₃₇FN₆O, calcd 577.3, found 577.2.

### Example 25: 2-{[(S)-3-Methyl-1-piperidyl]methyl}-6-{6-chloro-4-[1-methyl-4-(4-methyl-4H-1,2,4-triazol-3-yl)-5-pyrazolyl]-2-pyridyl}-4-cyclopropyl-1,6-dihydro-1,6-diaza-7-indenone

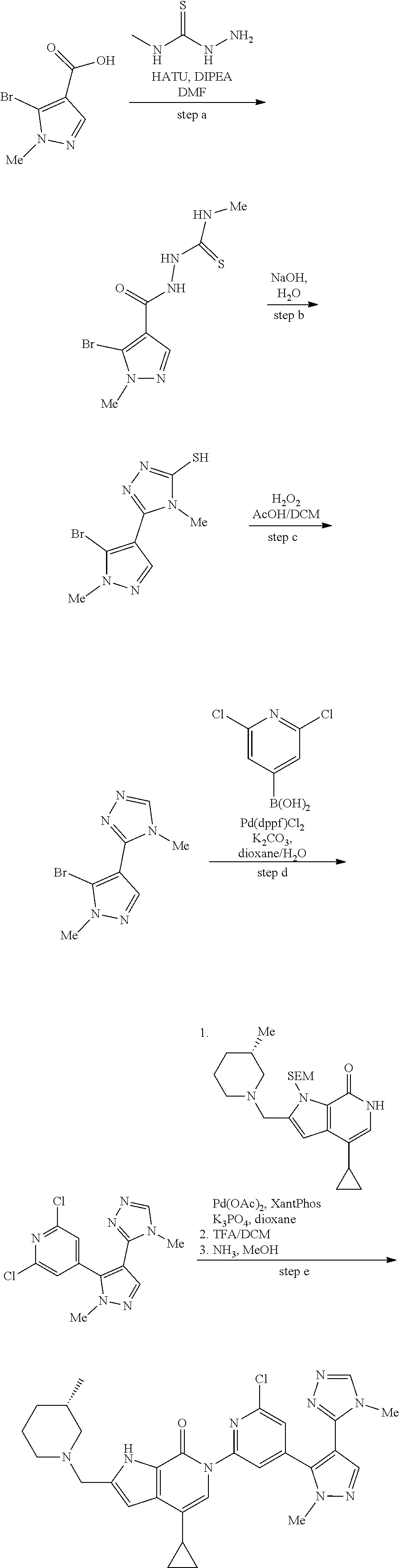

Step a: To a solution of 5-bromo-1-methylpyrazole-4-carboxylic acid (1.00 g, 4.9 mmol, 1.0 equiv.), 1-amino-3-methylthiourea (616 mg, 5.9 mmol, 1.2 equiv.) and HATU (2.05 g, 5.4 mmol, 1.1 equiv.) in DMF (8.0 mL) was added DIPEA (2.6 mL, 1.90 g, 15 mmol, 3.0 equiv.). The resulting mixture was stirred at room temperature for 3 h. Water (30 mL) was added to the reaction mixture causing the product to precipitate. The precipitate was collected by vacuum filtration, washed with water, and dried on the vacuum filtration funnel to constant weight. The obtained product was directly used for the next step.

Step b: The crude product from step a (˜4.9 mmol) was mixed with aq. 1M NaOH (15 mL). The resulting mixture was stirred at room temperature overnight. The reaction mixture was then neutralized with 1M HCl to pH˜4 inducing product precipitation. The precipitate was collected by vacuum filtration, washed with water, and dried on the vacuum filtration funnel to constant weight. The obtained material was directly used for the next step.

Step c: To a solution of the product from step b (˜4.9 mmol) in DCM (10 mL), was added acetic acid (5 mL) and aq. H₂O₂ (2.7 mL, 24 mmol, 5.0 equiv., 30 wt % solution) at 0° C. The resulting mixture was stirred at room temperature for 2 h, then quenched with saturated NaHCO₃ aqueous solution to pH˜8. The product was extracted with DCM/MeOH (10:1 v/v) mixture six times. The combined organic phase was dried over Na₂SO₄ and concentrated to yield crude product with sufficient purity for the next step.

Step d: To a mixture of the product from step c (1.00 g, 4.1 mmol, 1.0 equiv.) and (2,6-dichloropyridin-4-yl)boronic acid (789 mg, 4.1 mmol, 1.0 equiv.) in dioxane/H₂O (4:1 v/v, 25 mL) was added Pd(dppf)Cl₂ (300 mg, 0.41 mmol, 10 mol %) and K₂CO₃ (1.13 g, 8.2 mmol, 2.0 equiv.). The reaction mixture was heated at 100° C. under N₂ overnight before cooling to room temperature. The mixture was diluted with water and EtOAc. The organic phase was separated, and the aqueous layer was extracted with EtOAc. The combined organic extract was dried over Na₂SO₄, concentrated, and the crude residue was purified by reverse phase column chromatography (C18, MeCN in H₂O, 10 to 50%, 0.1% formic acid) to give the corresponding triaryl compound.

Step e: The reaction was performed in a similar fashion to that described for Example 10 to afford the title compound. ¹H NMR (400 MHz, CD₃OD) δ 8.49 (s, 1H), 8.44 (s, 1H), 7.95 (s, 1H), 7.86 (d, J=1.2 Hz, 1H), 7.56 (d, J=1.1 Hz, 1H), 7.25 (d, J=1.2 Hz, 1H), 6.72 (s, 1H), 4.17 (s, 2H), 4.03 (s, 3H), 3.63 (s, 3H), 3.27-3.17 (m, 2H), 2.57 (td, J=12.1, 3.0 Hz, 1H), 2.28 (t, J=11.6 Hz, 1H), 1.95 (ttd, J=8.3, 5.2, 1.2 Hz, 1H), 1.90-1.68 (m, 4H), 1.13-1.00 (m, 1H), 0.98-0.88 (m, 5H), 0.75-0.64 (m, 2H). ESI MS [M+H]⁺ for C₂₉H₃₂ClN₉O, calcd 558.2, found 558.2.

### Example 26: 6-{6-cyclopropyl-4-[4-fluoro-2-(4-methyl-4H-1,2,4-triazol-3-yl)phenyl]-2-pyridyl}-7-oxo-4-(trifluoromethyl)-1,6-dihydro-1,2,6-triaza-3-indenecarbonitrile

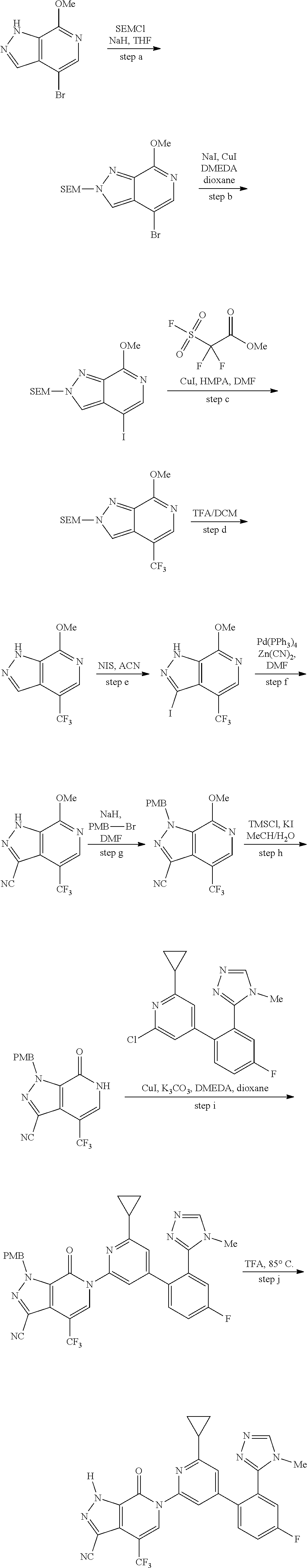

Step a: To a solution of 4-bromo-7-methoxy-1H-pyrazolo[3,4-c]pyridine (1.68 g, 7.4 mmol, 1.0 equiv.) in THE (30 mL) was added NaH (60 wt % in mineral oil, 0.44 g, 11.0 mmol, 1.5 equiv.) at 0° C. The resulting mixture was stirred at this temperature for 10 min before the addition of 2-(trimethylsilyl)ethoxymethyl chloride (1.35 g, 1.4 mL, 8.1 mmol, 1.1 equiv.). The reaction mixture temperature was then raised to room temperature and stirred for 1.5 h. LCMS and TLC showed full conversion to two regioisomeric products. The reaction was then quenched with water and diluted with EtOAc. The organic phase was separated, washed with brine, dried over Na₂SO₄ and concentrated. The crude residue was purified by column chromatography (SiO₂, EtOAc in hexanes, 0 to 25%) to give the desired isomer as product 2-[(4-bromo-7-methoxypyrazolo[3,4-c]pyridin-2-yl)methoxy]ethyl-trimethylsilane.

Step b: To a solution of the product from step a (1.3 mg, 3.64 mmol, 1.0 equiv.) in dioxane (25 mL) was added NaI (5.45 g, 36.4 mmol, 10.0 equiv.), CuI (695 mg, 3.64 mmol, 1.0 equiv.) and N,N′-dimethylethylenediamine (481 mg, 3.64 mmol, 1.0 equiv.). The resulting mixture was heated at 100° C. under N₂ overnight. After cooling to room temperature, the reaction mixture was filtered through a Celite® pad and concentrated. The residue was purified by column chromatography (SiO₂, EtOAc in hexanes, 0 to 20%) to give the desired product.

Step c: To a mixture of the product from step b (250 mg, 0.61 mmol, 1.0 equiv.), CuI (139 mg, 0.671 mmol, 1.1 equiv.) and HMIPA (546 mg, 3.05 mmol, 5.0 equiv.) in DMF (4 mL) was added methyl 2,2-difluoro-2-(fluorosulfonyl)acetate (585 mg, 3.05 mmol, 5.0 equiv.). The resulting mixture was heated at 80° C. under N₂ for 16 h. After cooling back to room temperature, the reaction mixture was diluted with EtOAc and water, then filtered through a Celite® pad. The filtrate was washed with H₂O and brine, dried over Na₂SO₄ and concentrated. The crude residue was purified by column chromatography (SiO₂, EtOAc in hexanes, 0 to 30%) to give the desired product.

Step d: The product from step c (246 mg, 0.71 mmol, 1.0 equiv.) was dissolved in 1:1 CH₂Cl₂/TFA (4 ml). The resulting solution was stirred at rt for 2 h. The solvent was removed, and NH₃ in MeOH (4 ml, excess, 7 N) was added. The reaction was stirred at rt for 1 h. The solvent was removed, and the dry residue was used in the next step without purification.

Step e: To a solution of the crude product from step d (218 mg, 0.71 mmol, 1.0 equiv.) in MeCN (4 mL) was added N-iodosuccinimide (319.3 mg, 1.42 mmol, 2.0 equiv.). The resulting mixture was stirred for 6 h when LCMS showed the completion of the iodination. The mixture was concentrated, and the crude product was purified by column chromatography (SiO₂, MeOH in DCM, 0 to 10%) to give the desired product.

Step f: To a solution of the product from step e (200 mg, 0.58 mmol, 1.0 equiv.) in DMF (3 mL) under N₂ was added Zn(CN)₂ (136.18 mg, 1.16 mmol, 2.0 equiv.) and Pd(PPh₃)₄ (66.9 mg, 0.058 mmol, 10 mol %). The resulting mixture was heated at 110° C. overnight. After cooling down to room temperature, the reaction mixture was diluted with EtOAc and filtered through Celite®, then washed with H₂O twice. The resulting organic solution was then concentrated, and the crude residue was purified by column chromatography (SiO₂, MeOH in DCM, 0 to 10%) to give the product.

Step g: To a solution of the product from step f (120 mg, 0.5 mmol, 1.0 equiv.) in DMF (2 mL) was added NaH (60 wt % in mineral oil, 40 mg, 1.0 mmol, 2.0 equiv.) at 0° C. The resulting mixture was stirred at this temperature for 10 min before the addition of 4-methoxybenzyl bromide (117 mg, 0.75 mmol, 1.5 equiv.). The reaction mixture was allowed to warm to room temperature and stirred for 3 h. The reaction was then quenched with water and diluted with EtOAc. The organic phase was separated, washed with brine, dried over Na₂SO₄ and concentrated. The crude residue was purified by column chromatography (SiO₂, EtOAc in hexanes, 0 to 45%) to give the desired product.

Step h: To a mixture of the product from step g (150 mg, 0.41 mmol, 1.0 equiv.) in MeCN (4.0 mL) was added TMSCl (71.2 mg, 0.65 mmol, 1.6 equiv.) and KI (265 mg, 0.65 mmol, 1.6 equiv.). The resulting mixture was stirred at room temperature for 2 h when LCMS showed completion of the demethylation. The reaction mixture was concentrated to dryness, and the crude product was purified by column chromatography (SiO₂, MeOH in DCM, 0 to 10%) to give the product.

Step i: To a solution of the product from step h (40 mg, 0.11 mmol, 1.0 equiv.) and 2-chloro-6-cyclopropyl-4-[4-fluoro-2-(4-methyl-1,2,4-triazol-3-yl)phenyl]pyridine (45 mg, 0.13 mmol, 1.2 equiv.) in dioxane (2.2 mL, 0.05 M) was added CuI (21 mg, 0.11 mmol, 1 equiv.), 1,2-dimethylethylenediamine (19.4 mg, 0.22 mmol, 2.0 equiv.) and K₂CO₃ (47.5 mg, 0.33 mmol, 3.0 equiv.). The resulting mixture was heated at 110° C. for 12 h. After cooling to room temperature the reaction mixture was diluted with EtOAc and washed with water twice. The organic phase was dried over Na₂SO₄ and concentrated. The dry residue was used for the next step without purification.

Step j: The crude product from step i was dissolved in trifluoroacetic acid and stirred at 85° C. for 12 h. The reaction mixture was concentrated to dryness, and the crude product was purified by reverse-phase HPLC to afford the title compound. ¹H NMR (400 MHz, CDCl₃) δ 8.64 (s, 1H), 8.06 (d, J=1.5 Hz, 1H), 7.65 (dd, J=8.6, 5.2 Hz, 1H), 7.49-7.39 (m, 2H), 7.31-7.29 (m, 1H), 7.24-7.20 (m, 1H), 3.32 (s, 3H), 2.15-2.08 (m, 1H), 1.14-1.02 (m, 4H); ESI MS [M+H]⁺ for C₂₅H₁₆F₄N₈O, calcd 521.1, found 521.0.

### Example 27: 6-{6-cyclopropyl-4-[4-fluoro-2-(4-methyl-4H-1,2,4-triazol-3-yl)phenyl]-2-pyridyl}-7-oxo-1,6-dihydro-1,2,6-triaza-3,4-indenedicarbonitrile

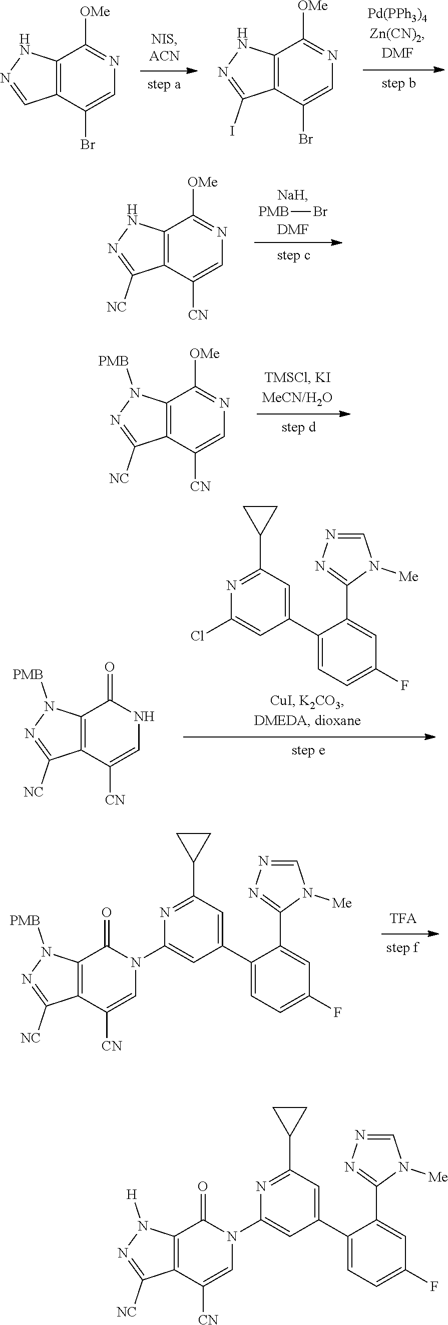

Step a: To a solution of 4-bromo-7-methoxy-1H-pyrazolo[3,4-c]pyridine (1.14 g, 5.0 mmol, 1.0 equiv.) in MeCN (4 mL) was added N-iodosuccinimide (1.46 g, 6.5 mmol, 1.5 equiv.). The resulting mixture was stirred for 2 h when LCMS showed the completion of the iodination. The mixture was concentrated, and the crude product was purified by column chromatography (SiO₂, MeOH in DCM, 0 to 10%) to give the desired product.

Step b: To a solution of the product from step b (706 mg, 2.0 mmol, 1.0 equiv.) in DMF (10 mL) under N₂ was added Zn(CN)₂ (1.4 g, 12.0 mmol, 6.0 equiv.) and Pd(PPh₃)₄ (462 mg, 0.2 mmol, 20 mol %). The resulting mixture was heated at 110° C. and stirred overnight. After cooling down to room temperature the reaction mixture was diluted with EtOAc and filtered through a Celite® pad, and then washed with H₂O twice. The resulting organic solution was then concentrated, and the crude residue was purified by column chromatography (SiO₂, MeOH in DCM, 0 to 10%) to give the desired product.

Step c: To a solution of the product from step b (199 mg, 1.0 mmol, 1.0 equiv.) in DMF (2.5 mL) was added NaH (60 wt % in mineral oil, 80 mg, 2.0 mmol, 2.0 equiv.) at 0° C. The resulting mixture was stirred at this temperature for 10 min before the addition of 4-methoxybenzyl bromide (234 mg, 1.5 mmol, 1.5 equiv.). The reaction mixture temperature was then raised to 23° C. and stirred for 3 h. The mixture was then quenched with water and diluted with EtOAc. The organic phase was separated, washed with brine, dried over Na₂SO₄ and concentrated. The crude residue was purified by column chromatography (SiO₂, EtOAc in hexanes, 0 to 45%) to give the desired product.

Step d: To a mixture of the product from step c (100 mg, 0.31 mmol, 1.0 equiv.) in MeCN (3.0 mL) was added TMSCl (54.1 mg, 0.49 mmol, 1.6 equiv.) and KI (82 mg, 0.49 mmol, 1.6 equiv.). The resulting mixture was stirred at room temperature for 2 h when LCMS showed complete transformation. The reaction mixture was concentrated to dryness, and the crude product was purified by column chromatography (SiO₂, MeOH in DCM, 0 to 10%) to give the product.

Step e: To a solution of the product from step d (35 mg, 0.11 mmol, 1.0 equiv.) and 2-chloro-6-cyclopropyl-4-[4-fluoro-2-(4-methyl-1,2,4-triazol-3-yl)phenyl]pyridine (49 mg, 0.15 mmol, 1.3 equiv.) in dioxane (2.2 mL, 0.05 M) was added CuI (21 mg, 0.11 mmol, 1 equiv.), 1,2-dimethylethylenediamine (19.4 mg, 0.22 mmol, 2.0 equiv.) and K₂CO₃ (47.5 mg, 0.33 mmol, 3.0 equiv.). The resulting mixture was heated at 110° C. for 12 h. After cooling to room temperature, the reaction mixture was diluted with EtOAc and washed with water twice. The organic phase was dried over Na₂SO₄ and concentrated. The dry residue was used in the next step without purification.

Step f: The crude product from step e was dissolved in TFA (2.2 mL) and stirred at 85° C. for 12 h in a sealed vial. The reaction mixture was concentrated to dryness, and the crude product was purified by reversed phase HPLC to afford the title compound. ¹H NMR (400 MHz, CDCl₃) δ 8.79 (s, 1H), 8.16 (s, 1H), 7.63 (dd, J=8.5, 5.2 Hz, 1H), 7.48-7.38 (m, 2H), 7.35-7.32 (m, 1H), 7.16 (s, 1H), 3.38 (s, 3H), 2.18-2.11 (m, 1H), 1.16-1.05 (m, 4H); ESI MS [M+H]⁺ for C₂₅H₁₆FN₉O, calcd 478.1, found 478.1.

### Example 28: 3-[6-cyclopropyl-4-[4-fluoro-2-(4-methyl-1,2,4-triazol-3-yl)phenyl]pyridin-2-yl]-6-[(2-methoxyethylamino)methyl]-5H-pyrrolo[3,2-d]pyrimidin-4-one

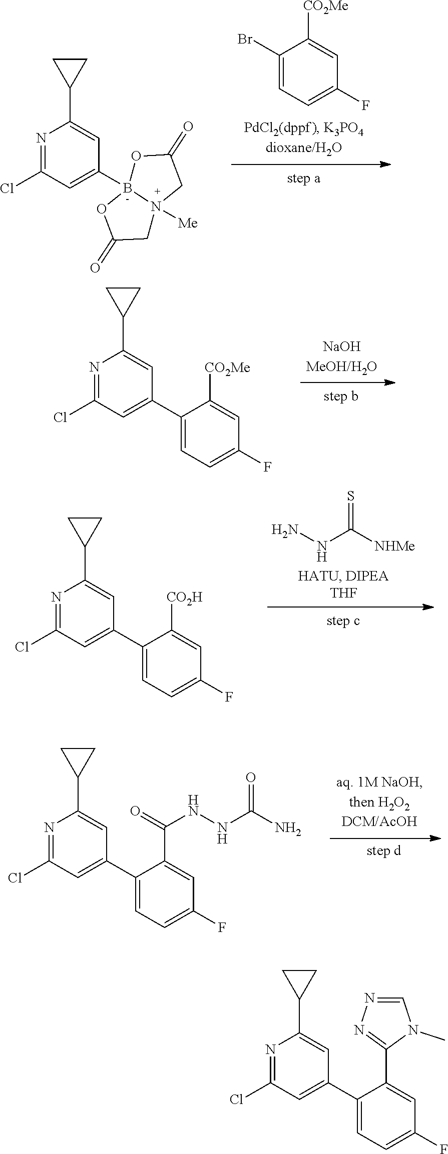

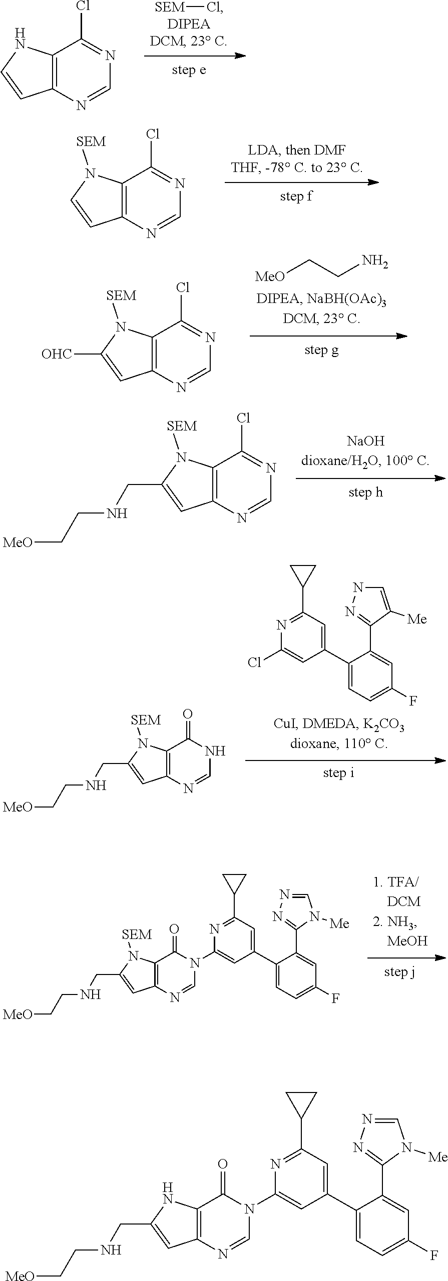

Step a: A suspension of 1-(2-chloro-6-cyclopropylpyridin-4-yl)-5-methyl-2,8-dioxa-5-azonia-1-boranuidabicyclo[3.3.0]octane-3,7-dione (9.8 g, 32 mmol, 1 equiv., prepared according to example 18), methyl 2-bromo-5-fluorobenzoate (7.8 g, 33 mmol, 1.05 equiv.) and K₃PO₄ (20.4 g, 96 mmol, 3.0 equiv.) in a 4:1 mixture of dioxane/water (320 mL) was degassed by applying vacuum followed by backfilling with nitrogen (repeated 3 times). Pd(dppf)Cl₂ (2.3 g, 3.2 mmol, 0.1 equiv.) was added and the reaction mixture was heated to 90° C. for 6 h. The reaction was cooled to room temperature and partitioned between EtOAc (300 mL) and brine (500 mL). The aqueous phase was extracted with EtOAc (2×200 mL) and the combined organics were dried over Na₂SO₄, filtered, and concentrated under vacuum. The crude residue was purified by column chromatography (SiO₂, 0 to 100% EtOAc in hexanes) to afford the desired product.

Step b: To a solution of the product from step a (8.3 g, 27.0 mmol, 1.0 equiv.) in MeOH (108 mL) was added NaOH (5.4 g, 136 mmol, 5 equiv.) as a solution in water (27 mL). The resulting solution was stirred at 23° C. for 2 hours. The mixture was acidified with aqueous 1 M HCl to pH˜1, and the product was extracted with EtOAc (3×200 mL). The combined organics were dried over Na₂SO₄ and concentrated under vacuum to afford the desired product.

Step c: HATU (9.7 g, 26 mmol, 1.4 equiv.) was added to a suspension of the product from step b (5.3 g, 18.2 mmol, 1 equiv.), 4-methyl-3-thiosemicarbazide (2.3 g, 22.0 mmol, 1.2 equiv.) and DIPEA (9.5 mL, 55.0 mmol, 3.0 equiv.) in THE (60 mL). The reaction mixture was stirred for 3 hours at 23° C. at which point it was directly concentrated under vacuum. The crude residue was triturated with water (300 mL) and the resulting precipitate was washed with water (2×100 mL) and MTBE (2×50 mL) and dried on the filter.

Step d: The product from step c (6.1 g, 16.1 mmol) was suspended in aq. 1M NaOH (160 mL) and heated at 65° C. for 16 hours. The reaction mixture was cooled to room temperature and acidified with aqueous 1 M HCl to pH˜1. The resulting precipitate was collected by filtration, washed with water (50 mL), and dried on the frit. The crude material was suspended in a mixture of AcOH (9 mL) and dichloromethane (72 mL), and the reaction mixture was cooled to 0° C. H₂O₂ (5 mL, 48.3 mmol, 3 equiv.) was added and the mixture was stirred at 0° C. for 20 min. The cooling bath was removed, and the reaction was stirred for an additional 2 hours at room temperature. The reaction mixture was diluted with dichloromethane (500 mL) and washed with water (3×200 mL) and saturated aqueous NaHCO₃ (200 mL), dried over Na₂SO₄ and concentrated under vacuum. The crude residue was purified by reversed phase column chromatography (SiO₂-C18, 0 to 100% MeCN in water with 0.1% formic acid) to afford the desired product.

Step e: To a solution of 4-chloro-5H-pyrrolo[3,2-d]pyrimidine (8.5 g, 55.3 mmol, 1.0 equiv.) in dichloromethane (220 mL) was added DIPEA (14.5 mL, 83.0 mmol, 1.5 equiv.) followed by SEM-Cl (10.8 mL, 60.8 mmol, 1.1 equiv.). The reaction mixture was stirred at room temperature for 1 hour at which point it was quenched with a 1:1 mixture of water/brine (500 mL) and extracted with dichloromethane (200 mL). The combined organics were dried over Na₂SO₄, filtered, and concentrated under vacuum. The crude residue was purified by column chromatography (SiO₂, EtOAc in hexanes, 0 to 60%) to afford the desired product.

Step f: LDA (2.0 M in THF, 3.3 mL, 6.0 mmol, 1.25 equiv.) was added to THE (27 mL) and the resulting solution was cooled to −78° C. The product of step e (1.5 g, 5.3 mmol, 1.0 equiv.) was added dropwise to the reaction mixture as a solution in THE (5 mL). The reaction mixture was stirred at −78° C. for 1.5 hours, at which point DMF (0.65 mL, 7.9 mmol, 1.5 equiv.) was added dropwise to the reaction mixture. The reaction was stirred for an additional 30 minutes at −78° C., the dry ice bath was removed, and the reaction was allowed to warm to room temperature and stir for 30 minutes. The reaction was quenched with saturated aqueous NH₄Cl (20 mL) and partitioned between EtOAc (100 mL) and H₂O (100 mL). The aqueous phase was extracted with EtOAc (100 mL) and the combined organics were dried over Na₂SO₄, filtered, and concentrated under vacuum. The crude residue was purified by column chromatography (SiO₂, EtOAc in hexanes, 0 to 100%) to afford the desired product.

Step g: To a solution of the product of step f (200 mg, 0.64 mmol, 1.0 equiv.) in dichloromethane (6 mL) was added 2-methoxyethanamine (55 uL, 0.64 mmol, 1.0 equiv.) and DIPEA (225 uL, 1.30 mmol, 2.0 equiv.). The reaction mixture was stirred for 5 minutes at room temperature and NaBH(OAc)₃ (203 mg, 0.96 mmol, 1.5 equiv.) was added. The reaction was stirred for an additional 16 hours at room temperature at which point it was quenched with saturated aqueous NaHCO₃ (50 mL) and extracted with dichloromethane (50 mL). The combined organics were dried over Na₂SO₄, filtered, and concentrated under vacuum. The crude residue was purified by column chromatography (SiO₂, MeOH in dichloromethane, 0 to 20%) to afford the desired product.

Step h: To a solution of the product from step g (180 mg, 0.49 mmol, 1.0 equiv.) in dioxane (2.5 mL) was added 1.0 M aqueous NaOH (2.5 mL). The reaction mixture was heated to 100° C. and stirred for 16 hours at which point it was quenched with saturated aqueous NH₄Cl (25 mL) and extracted with EtOAc (2×10 mL). The combined organics were dried over Na₂SO₄, filtered, and concentrated under vacuum. The crude material was used directly in the next step without further purification.

Step i: To a suspension of the product from step h (75 mg, 0.21 mmol, 1.0 equiv.), the product of step f (69 mg, 0.21 mmol, 1.0 equiv.), and K₂CO₃ (87 mg, 0.63 mmol, 3.0 equiv.) in dioxane (4.2 mL) was added CuI (40 mg, 0.21 mmol, 1.0 equiv.) and DMEDA (45 uL, 0.42 mmol, 2.0 equiv.). The reaction mixture was heated to 110° C. in a sealed vial and stirred for 16 hours at which point it was quenched with a 1:1 mixture of water/brine (20 mL) and extracted with EtOAc (2×10 mL). The combined organics were dried over Na₂SO₄, filtered, and concentrated under vacuum. The crude residue was purified by column chromatography (SiO₂, MeOH in dichloromethane, 0 to 20%) to afford the desired product.

Step j: To a solution of the product from step i (34 mg, 0.05 mmol, 1.0 equiv.) in dichloromethane (1 mL) was added TFA (1 mL). The reaction mixture was heated to 30° C. and stirred for 1 hour at which point it was diluted with toluene (5 mL) and concentrated under vacuum. The crude residue was dissolved in 7M NH₃ in MeOH (2 mL) and the resulting reaction mixture was stirred at 30° C. for 1 hour. The reaction was directly concentrated and purified by reverse phase prep-HPLC (C18 column, 5 to 50% MeCN/H₂O) to afford the desired product. ¹H NMR (400 MHz, CDCl₃) δ 8.33 (s, 1H), 7.99 (s, 1H), 7.66 (dd, J=8.6, 5.3 Hz, 1H), 7.49 (dd, J=8.5, 2.7 Hz, 1H), 7.40 (td, J=8.2, 2.7 Hz, 1H), 7.20 (d, J=1.4 Hz, 1H), 7.18 (s, 1H), 6.54 (s, 1H), 4.55 (s, 2H), 3.96 (t, J=4.9 Hz, 2H), 3.48 (t, J=4.8 Hz, 2H), 3.44 (s, 3H), 3.11 (s, 3H), 2.01 (p, J=6.8 Hz, 1H), 1.01-0.95 (m, 4H). ESI MS [M+H]+ for C₂₇H₂₇FN₈O₂, calcd 515.2, found 515.2.

### Example 29: 3-[6-cyclopropyl-4-[4-fluoro-2-(4-methyl-1,2,4-triazol-3-yl)phenyl]pyridin-2-yl]-6-[[(1-methylcyclobutyl)amino]methyl]-5H-pyrrolo[3,2-d]pyrimidin-4-one

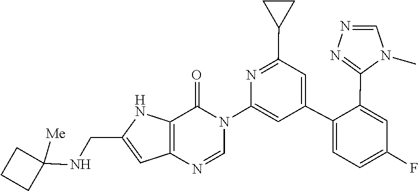 [SMILES: Cn1cnnc1-c1cc(F)ccc1-c1cc(C2CC2)nc(-n2cnc3cc(CNC4(C)CCC4)[nH]c3c2=O)c1]

The title compound was prepared in a similar fashion to that described for example 28 using 1-methylcyclobutan-1-amine for reductive amination step. ¹H NMR (400 MHz, CDCl₃) δ 8.26 (s, 1H), 8.12 (s, 1H), 7.59 (dd, J=8.6, 5.4 Hz, 1H), 7.45-7.32 (m, 3H), 6.91 (d, J=1.3 Hz, 1H), 6.34 (s, 1H), 3.90 (s, 2H), 3.15 (s, 3H), 2.05-1.70 (m, 7H), 1.25 (s, 3H), 1.07-0.90 (m, 4H). ESI MS [M+H]+ for C₂₉H₂₉FN₈O, calcd 525.3, found 525.3.

### Example 30: 6-{6-cyclopropyl-4-[4-fluoro-2-(4-methyl-4H-1,2,4-triazol-3-yl)phenyl]-2-pyridyl}-2-[(2-trifluoromethoxyethylamino)methyl]-1,6-dihydro-1,4,6-triaza-7-indenone

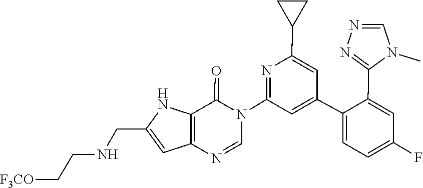 [SMILES: Cn1cnnc1-c1cc(F)ccc1-c1cc(C2CC2)nc(-n2cnc3cc(CNCCOC(F)(F)F)[nH]c3c2=O)c1]

The title compound was prepared in a similar fashion to that described for example 28 using 2-(trifluoromethoxy)ethanamine for reductive amination step. ¹H NMR (400 MHz, CDCl₃) δ 8.28 (s, 1H), 8.15 (s, 1H), 7.60 (dd, J=8.6, 5.4 Hz, 1H), 7.43-7.33 (m, 3H), 6.97 (d, J=1.4 Hz, 1H), 6.39 (s, 1H), 4.13-4.05 (m, 2H), 4.02 (s, 2H), 3.16 (s, 3H), 2.94 (t, J=5.1 Hz, 2H), 2.01-1.95 (m, 1H), 1.03-0.95 (m, 4H). ESI MS [M+H]⁺ C₂₇H₂₄F₄N₈O₂, calcd 569.2, found 569.2.

### Example 31: 6-{6-cyclopropyl-4-[4-fluoro-2-(4-methyl-4H-1,2,4-triazol-3-yl)phenyl]-2-pyridyl}-2-[(2-ethoxyethylamino)methyl]-1,6-dihydro-1,4,6-triaza-7-indenone

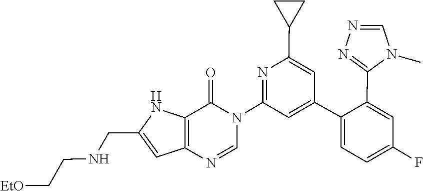 [SMILES: CCOCCNCc1cc2ncn(-c3cc(-c4ccc(F)cc4-c4nncn4C)cc(C4CC4)n3)c(=O)c2[nH]1]

The title compound was prepared in a similar fashion to that described for example 28 using 2-ethoxyethanamine for reductive amination step. ¹H NMR (400 MHz, CDCl₃) δ 8.25 (s, 1H), 8.15 (s, 1H), 7.63-7.55 (m, 1H), 7.45-7.34 (m, 3H), 6.95 (d, J=1.4 Hz, 1H), 6.40 (s, 1H), 4.10 (s, 2H), 3.64-3.58 (m, 2H), 3.52 (q, J=7.0 Hz, 2H), 3.16 (s, 3H), 2.92 (t, J=5.0 Hz, 2H), 2.00-1.90 (m, 1H), 1.21 (t, J=7.0 Hz, 3H), 1.02-0.91 (m, 4H). ESI MS [M+H]⁺ C₂₈H₂₉FN₈O₂, calcd 529.2, found 529.2.

### Example 32: 6-{6-cyclopropyl-4-[4-fluoro-2-(4-methyl-4H-1,2,4-triazol-3-yl)phenyl]-2-pyridyl}-2-[(3-methoxy-1-azetidinyl)methyl]-1,6-dihydro-1,4,6-triaza-7-indenone

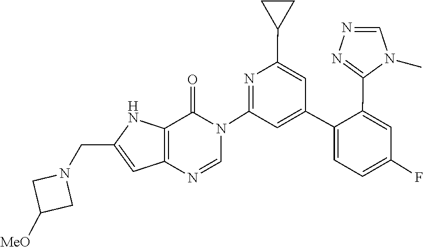 [SMILES: COC1CN(Cc2cc3ncn(-c4cc(-c5ccc(F)cc5-c5nncn5C)cc(C5CC5)n4)c(=O)c3[nH]2)C1]

The title compound was prepared in a similar fashion to that described for example 28 using 3-methoxyazetidine for reductive amination step. ¹H NMR (400 MHz, CDCl₃) δ 12.29 (s, 1H), 8.37 (s, 1H), 8.25 (s, 1H), 7.62 (dd, J=9.5, 5.3 Hz, 1H), 7.42-7.34 (m, 2H), 7.23 (s, 1H), 7.10 (s, 1H), 6.56 (s, 1H), 4.51 (s, 2H), 4.47-4.32 (m, 3H), 3.87 (s, 2H), 3.32 (s, 3H), 3.25 (s, 3H), 2.05-1.97 (m, 1H), 1.05-0.95 (m, 4H). ESI MS [M+H]⁺ C₂₈H₂₇FN₈O₂, calcd 527.2, found 527.2.

### Example 33: 6-{6-cyclopropyl-4-[4-fluoro-2-(4-methyl-4H-1,2,4-triazol-3-yl)phenyl]-2-pyridyl}-2-[(2,2,2-trifluoroethylamino)methyl]-1,6-dihydro-1,4,6-triaza-7-indenone

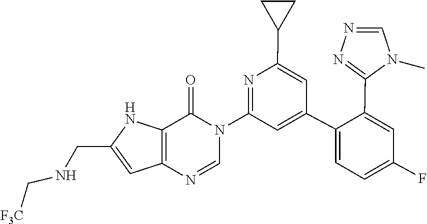 [SMILES: Cn1cnnc1-c1cc(F)ccc1-c1cc(C2CC2)nc(-n2cnc3cc(CNCC(F)(F)F)[nH]c3c2=O)c1]

The title compound was prepared in a similar fashion to that described for example 28 using 2,2,2-trifluoroethylamine for reductive amination step. ¹H NMR (400 MHz, CDCl₃) δ 8.27 (s, 1H), 8.21-8.09 (m, 1H), 7.60 (dd, J=8.6, 5.4 Hz, 1H), 7.45-7.29 (m, 3H), 6.99 (s, 1H), 6.42 (s, 1H), 4.11 (s, 2H), 3.16 (s, 5H), 2.04-1.95 (m, 1H), 1.06-0.93 (m, 4H). ESI MS [M+H]⁺ C₂₆H₂₂F₄N₈O, calcd 539.2, found 539.2.

### Example 34: 3-[6-cyclopropyl-4-[4-fluoro-2-(4-methyl-1,2,4-triazol-3-yl)phenyl]pyridin-2-yl]-6-[[2-methoxyethyl(methyl)amino]methyl]-5H-pyrrolo[3,2-d]pyrimidin-4-one

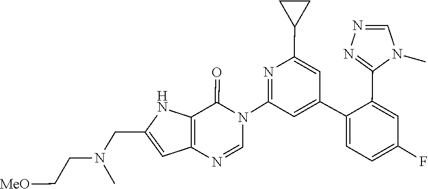 [SMILES: COCCN(C)Cc1cc2ncn(-c3cc(-c4ccc(F)cc4-c4nncn4C)cc(C4CC4)n3)c(=O)c2[nH]1]

The title compound was prepared in a similar fashion to that described for example 28 using 2-methoxy-N-methylethanamine for reductive amination step. ¹H NMR (400 MHz, CDCl₃) δ 10.30 (s, 1H), 8.29 (s, 1H), 8.10 (s, 1H), 7.59 (dd, J=8.6, 5.4 Hz, 1H), 7.47-7.32 (m, 3H), 6.89 (d, J=1.4 Hz, 1H), 6.35 (s, 1H), 3.81 (s, 2H), 3.56 (t, J=5.1 Hz, 2H), 3.42 (s, 3H), 3.16 (s, 3H), 2.68 (t, J=5.1 Hz, 2H), 2.36 (s, 3H), 2.18-1.81 (m, 1H), 1.02-0.91 (m, 4H). ESI MS [M+H]⁺ C₂₈H₂₉FN₈O₂, calcd 529.2, found 529.2.

### Example 35: 6-{6-cyclopropyl-4-[4-fluoro-2-(4-methyl-4H-1,2,4-triazol-3-yl)phenyl]-2-pyridyl}-2-{[(2-methoxyethyl)(2,2,2-trifluoroethyl)amino]methyl}-1,6-dihydro-1,4,6-triaza-7-indenone

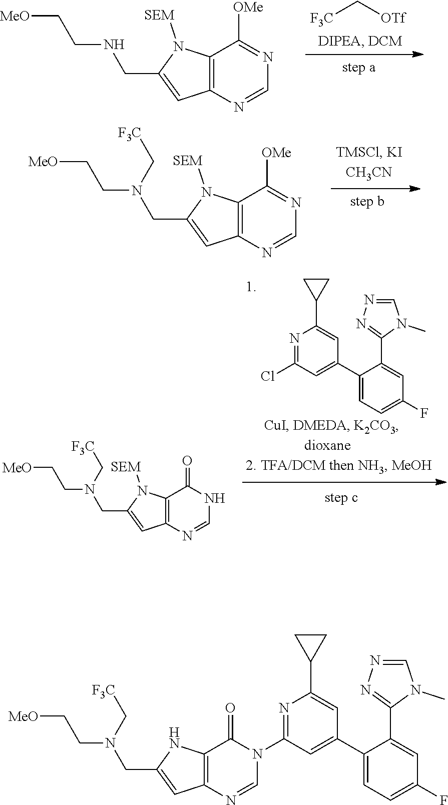 [SMILES: COCCNCc1cc2ncnc(OC)c2n1COCC[Si](C)(C)C ; O=S(=O)(OCC(F)(F)F)C(F)(F)F ; COCCN(Cc1cc2ncnc(OC)c2n1COCC[Si](C)(C)C)CC(F)(F)F ; Cn1cnnc1-c1cc(F)ccc1-c1cc(Cl)nc(C2CC2)c1 ; COCCN(Cc1cc2nc[nH]c(=O)c2n1COCC[Si](C)(C)C)CC(F)(F)F ; COCCN(Cc1cc2ncn(-c3cc(-c4ccc(F)cc4-c4nncn4C)cc(C4CC4)n3)c(=O)c2[nH]1)CC(F)(F)F]

Step a: To a mixture of 2-methoxy-N-[[4-methoxy-5-(2-trimethylsilylethoxymethyl)pyrrolo[3,2-d]pyrimidin-6-yl]methyl]ethanamine (400 mg, 1.09 mmol, 1.0 equiv., prepared according to example 22, step d, from methoxyethanamine) and 2,2,2-trifluoroethyl trifluoromethanesulfonate (378.3 mg, 1.63 mmol, 1.5 equiv) in dichloromethane (3 mL), was added DIPEA (0.56 mL, 3.27 mmol, 3.0 equiv). The resulting mixture was stirred at room temperature for 12 h. The reaction mixture was diluted with EtOAc (50 mL) and water (25 mL). The organic phase was separated, and the aqueous layer was additionally extracted with EtOAc (2×25 mL). The combined organic extract was dried over Na₂SO₄ and concentrated. The crude product was purified by column chromatography (SiO₂, 0-50% EtOAc gradient in hexanes) to afford the desired product.

Step b: The reaction was performed in a similar fashion to example 22, step e.

Step c: The reaction was performed in a similar fashion to example 22, steps f and g, using 2-chloro-6-cyclopropyl-4-[4-fluoro-2-(4-methyl-1,2,4-triazol-3-yl)phenyl]pyridine (prepared according to example 28). ¹H NMR (400 MHz, CDCl₃) δ 10.25 (s, 1H), 8.29 (s, 1H), 8.10 (s, 1H), 7.59 (dd, J=8.6, 5.4 Hz, 1H), 7.47-7.31 (m, 3H), 6.88 (d, J=1.3 Hz, 1H), 6.35 (d, J=2.1 Hz, 1H), 4.10 (s, 2H), 3.58-3.52 (m, 2H), 3.46 (s, 3H), 3.25 (q, J=9.3 Hz, 2H), 3.16 (s, 3H), 2.96 (t, J=4.9 Hz, 2H), 1.98-1.88 (m, 1H), 1.01-0.93 (m, 4H). ESI MS [M+H]⁺ C₂₉H₂₈F₄N₈O₂, calcd 597.2, found 597.2.

### Example 36: 6-{6-cyclopropyl-4-[4-fluoro-2-(4-methyl-4H-1,2,4-triazol-3-yl)phenyl]-2-pyridyl}-2-[2,2,2-trifluoro-1-(2-methoxyethylamino)ethyl]-1,6-dihydro-1,4,6-triaza-7-indenone

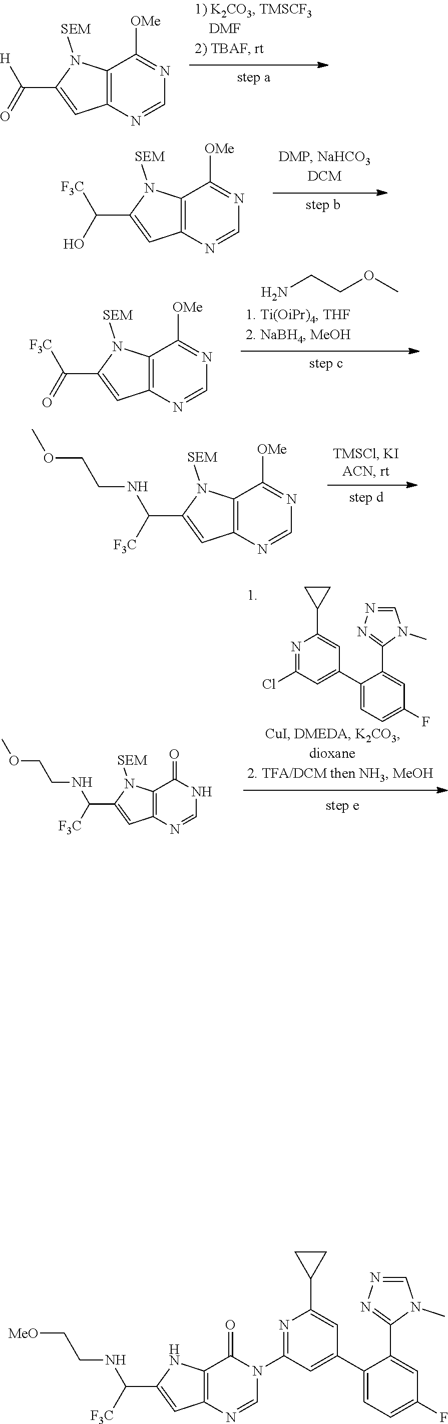

Step a: To mixture of the 4-methoxy-5-(2-trimethylsilylethoxymethyl)pyrrolo[3,2-d]pyrimidine-6-carbaldehyde (4.0 g, 13.2 mmol, 1.0 equiv., prepared according to example 22) and K₂CO₃ (18 mg, 0.13 mmol, 0.01 equiv.) in DMF was added TMSCF₃ (3.8 mL, 26.04 mmol, 2.0 equiv.). The resulting mixture was stirred at room temperature for 3 h and then concentrated under vacuum. The crude residue was dissolved in THF (13 mL), and TBAF (1 M in THF, 13 mL) was added. The resulting mixture was stirred at room temperature for 30 minutes, then diluted with EtOAc (50 mL) and water (25 mL). The organic phase was separated, and the aqueous layer was additionally extracted with EtOAc (2×50 mL). The combined organic extract was dried over Na₂SO₄ and concentrated to dryness under reduced pressure. The crude product was purified by column chromatography (SiO₂, 0-80% EtOAc gradient in hexanes) to afford the desired product.

Step b: To a solution of the product from step a (0.52 g, 1.4 mmol, 1.0 equiv.) in dichloromethane (7 mL, 0.2 M) were sequentially added NaHCO₃ (0.47 g, 5.6 mmol, 4.0 equiv.) and DMP (0.71 g, 1.68 mmol, 3.0 equiv.). The resulting mixture was stirred at 23° C. for 4 h. The resulting mixture was diluted with dichloromethane followed by quench with aq. sat. Na₂S₂O₃ (5 mL) and aq. sat. NaHCO₃ (10 mL). The resulting biphasic mixture was stirred for 1 h at 23° C. before the organic layer was separated. The aqueous layer was extracted with DCM (2×30 mL). The combined organic phase was dried over Na₂SO₄ and concentrated under reduced pressure. The crude residue was purified by column chromatography (SiO₂, 0-80% EtOAc gradient in hexanes) to afford the desired ketone product.

Step c: To a solution of the product from step b (0.25 g, 0.67 mmol, 1.0 equiv) and methoxyethanamine (0.10 g, 1.34 mmol, 2.0 equiv.) in THE (6.7 mL, 0.1 M) was added Ti(Oi-Pr)₄ (0.95 g, 3.35 mmol, 5.0 equiv.) at room temperature. The obtained mixture was stirred at 60° C. for 3 h, then cooled to room temperature, and NaBH₄ (50.6 mg, 1.34 mmol, 2.0 equiv.). After stirring for 3 h at 23° C. the reaction was diluted with MeOH (1 mL) and concentrated to dryness under reduced pressure. The dry residue was partitioned between CH₂Cl₂ (40 mL) and aq. sat. NaHCO₃ (10 mL). The organic phase was separated, and the aqueous phase was additionally extracted with CH₂Cl₂ (2×25 mL). The combined organic extract was washed with water, dried over Na₂SO₄, filtered, and the solvent was removed under reduced pressure. The crude residue was purified by column chromatography (SiO₂, 0-80% EtOAc gradient in hexanes) to afford the reductive amination product.

Step d: The reaction was performed in a similar fashion to example 22, step e to afford 6-[2,2,2-trifluoro-1-(2-methoxyethylamino)ethyl]-5-(2-trimethylsilylethoxymethyl)-3H-pyrrolo[3,2-d]pyrimidin-4-one as the desired product.

Step e: The reaction was performed in a similar fashion to example 28, steps i and j, using the product from step d and 2-chloro-6-cyclopropyl-4-[4-fluoro-2-(4-methyl-1,2,4-triazol-3-yl)phenyl]pyridine. ¹H NMR (400 MHz, CDCl₃) δ 10.25 (s, 1H), 8.30 (s, 1H), 8.13 (s, 1H), 7.59 (dd, J=8.6, 5.4 Hz, 1H), 7.45-7.34 (m, 3H), 6.97 (d, J=1.4 Hz, 1H), 6.60 (d, J=2.0 Hz, 1H), 4.52 (q, J=7.0 Hz, 1H), 3.54-3.45 (m, 2H), 3.39 (s, 3H), 3.16 (s, 3H), 2.98-2.72 (m, 2H), 2.03-1.90 (m, 1H), 1.77 (s, 1H), 1.04-0.91 (m, 4H). C₂₈H₂₆F₄N₈O₂, calcd 583.2, found 583.2.

### Example 37: 4-(6-cyclopropyl-2-{4-cyclopropyl-2-[(1-methylcyclobutylamino)methyl]-7-oxo-1,6-dihydro-1,6-diaza-6-indenyl}-4-pyridyl)-3-(4-methyl-4H-1,2,4-triazol-3-yl)benzonitrile

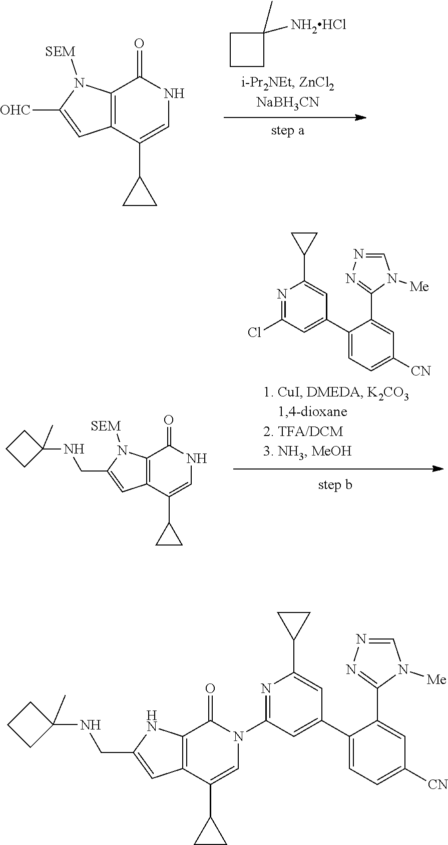

Step a: To the solution of 4-cyclopropyl-7-oxo-1-(2-trimethylsilylethoxymethyl)-6H-pyrrolo[2,3-c]pyridine-2-carbaldehyde (0.5 g, 2.25 mmol, 1.0 equiv., prepared according to example 1) in dichloromethane (5 mL, 0.45 M) was added 1-methylcyclobutan-1-amine hydrochloride (0.55 g, 4.50 mmol, 2.0 equiv.), N,N-diisopropylethylamine (1.2 mL, 6.6 mmol, 3.0 equiv.) and ZnCl₂ (1.53 g, 11.3 mmol, 5.0 equiv.). The resulting mixture was stirred at room temperature for 1 h before NaBH₃CN (0.29 g, 4.50 mmol, 2.0 equiv.) was added. The mixture was stirred at room temperature for 16 h, then diluted with dichloromethane (5 ml) and quenched with aqueous saturated NaHCO₃ (5 mL). The organic phase was separated, and the aqueous layer was extracted with EtOAc (2×5 mL). The combined organic phase was dried over Na₂SO₄, and the solvent was removed under vacuum. The crude residue was purified by column chromatography (SiO₂, MeOH in dichloromethane, 0 to 10%) to afford 4-cyclopropyl-2-[[(1-methylcyclobutyl)amino]methyl]-1-(2-trimethylsilylethoxymethyl)-6H-pyrrolo[2,3-c]pyridin-7-one.

Step b: To a mixture of the product from step a (81.4 mg, 0.20 mmol, 1.0 equiv.), 4-(2-chloro-6-cyclopropylpyridin-4-yl)-3-(4-methyl-1,2,4-triazol-3-yl)benzonitrile (68.0 mg, 0.20 mmol, 1.0 equiv., prepared according to example 2) in dioxane (3.0 mL) was added CuI (38.5 mg, 0.20 mmol, 1.0 equiv.), N,N′-dimethylethylenediamine (71.2 mg, 0.81 mmol, 4.0 equiv.) and K₂CO₃ (83.8 mg, 0.61 mmol, 3.0 equiv.) After the reaction mixture was purged with nitrogen for 10 min it was heated at 110° C. overnight. The resulting mixture was cooled to room temperature, diluted with EtOAc (7 mL), and sequentially washed with aqueous saturated NH₄Cl (10 ml) and water (10 mL). The organic phase was separated, dried over Na₂SO₄ and concentrated to dryness under reduced pressure. The residual material was dissolved in a mixture of trifluoroacetic acid/dichloromethane (2 mL, 1:10 v/v) at room temperature for 3 h. The volatiles were removed under reduced pressure, and the dry residue was dissolved in 7M NH₃ in methanol (2 mL). The obtained solution was stirred for 30 min at room temperature followed by solvent removal under vacuum. The crude product was purified by prep-HPLC (C18 SiO₂, 10-100% CH₃CN in water with 0.1% formic acid) to afford title compound. ¹H NMR (400 MHz, CDCl₃) δ 8.10 (s, 1H), 7.96 (d, J=1.6 Hz, 1H), 7.90 (dd, J=8.1, 1.7 Hz, 1H), 7.69 (d, J=8.1 Hz, 1H), 7.60 (d, J=1.5 Hz, 1H), 7.18 (d, J=1.2 Hz, 1H), 6.78 (d, J=1.4 Hz, 1H), 6.32 (s, 1H), 3.93 (s, 2H), 3.16 (s, 3H), 2.30-2.12 (m, 2H), 1.96 (tt, J=8.0, 4.8 Hz, 1H), 1.90-1.71 (m, 4H), 1.39 (s, 3H), 1.00 (dt, J=8.0, 2.9 Hz, 2H), 0.95 (dt, J=5.3, 2.9 Hz, 2H), 0.91-0.83 (m, 2H), 0.67-0.61 (m, 2H). ESI MS [M+H]⁺ for C₃₄H₃₅N₈O, calcd 571.3, found 571.1.

### Example 38: 6-{6-cyclopropyl-4-[4-fluoro-2-(4-methyl-4H-1,2,4-triazol-3-yl)phenyl]-2-pyridyl}-2-[(2-methoxyethoxy)methyl]-1,6-dihydro-1,4,6-triaza-7-indenone

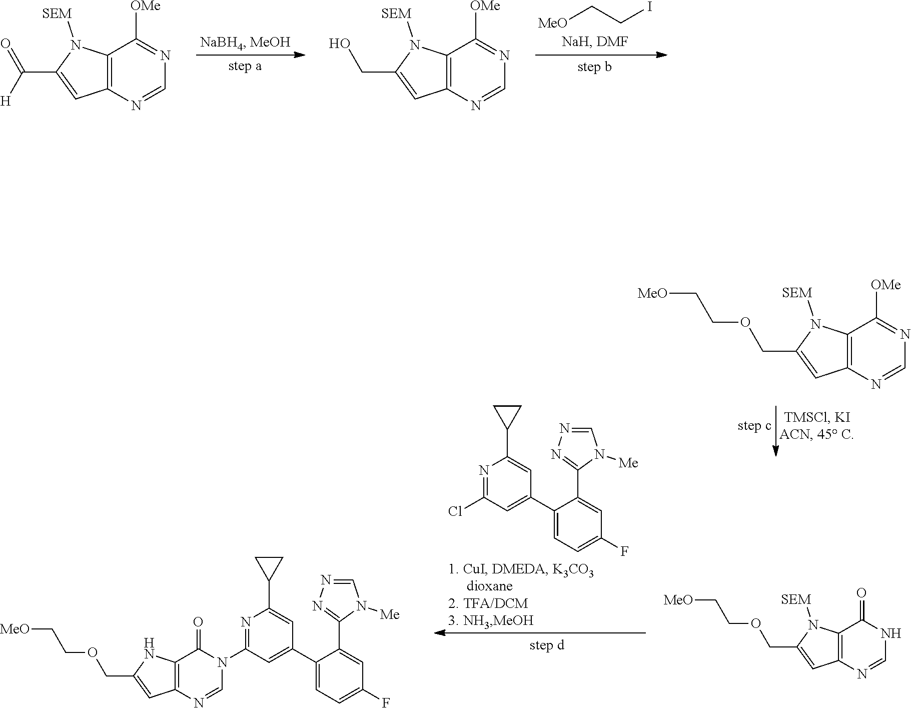

Step a: To a solution of the 4-methoxy-5-(2-trimethylsilylethoxymethyl)pyrrolo[3,2-d]pyrimidine-6-carbaldehyde (1.0 g, 3.25 mmol, 1 equiv., prepared according to example 22) in MeOH (16 mL) was added NaBH₄ (0.24 g, 6.5 mmol, 2.0 equiv.). The resulting mixture was stirred at room temperature for 4 h before addition of water (20 mL) and EtOAc (50 mL). The organic phase was separated, and the aqueous layer was additionally extracted with EtOAc (30 mL). The combined organic solution was washed with brine (30 mL), dried over Na₂SO₄ and concentrated to dryness under reduced pressure. The crude residue was purified by column chromatography (SiO₂, 0-80% EtOAc gradient in hexanes) to afford the desired product.

Step b: To a solution of the product from step a (250 mg, 0.8 mmol, 1.0 mmol) and 1-iodo-2-methoxyethane (146 mg, 2.4 mmol, 3.0 equiv) in DMF (4 mL) was added NaH (64 mg, 1.6 mmol, 2.0 equiv.) at 0° C. The resulting mixture was stirred at room temperature for 24 h before quench with water (20 mL). The product was extracted with EtOAc (2×30 mL). The combined organic phase was sequentially washed with water (30 mL), brine (30 mL), dried over Na₂SO₄ and concentrated to dryness under reduced pressure. The crude residue was purified by column chromatography (SiO₂, 0-80% EtOAc gradient in hexanes) to afford corresponding alkylation product.

Step c: The reaction was performed in a similar fashion to example 22, step e to afford 6-(2-methoxyethoxymethyl)-5-(2-trimethylsilylethoxymethyl)-3H-pyrrolo[3,2-d]pyrimidin-4-one.

Step d: The reaction was performed in a similar fashion to example 28, steps i and j using 2-chloro-6-cyclopropyl-4-[4-fluoro-2-(4-methyl-1,2,4-triazol-3-yl)phenyl]pyridine. ¹H NMR (400 MHz, CDCl₃) δ 10.19 (s, 1H), 8.29 (s, 1H), 8.13 (s, 1H), 7.60 (dd, J=8.6, 5.4 Hz, 1H), 7.48-7.32 (m, 3H), 6.92 (d, J=1.4 Hz, 1H), 6.39 (d, J=2.1 Hz, 1H), 4.74 (s, 2H), 3.75-3.69 (m, 2H), 3.64-3.59 (m, 2H), 3.45 (s, 3H), 3.16 (s, 3H), 1.99-1.92 (m, 1H), 1.03-0.93 (m, 4H). ESI MS [M+H]⁺ C₂₇H₂₆FN₇O₃, calcd 516.2, found 516.2.

### Example 39: 2-cyclopropyl-5-[6-cyclopropyl-4-[4-fluoro-2-(4-methyl-1,2,4-triazol-3-yl)phenyl]pyridin-2-yl]-7-(trifluoromethyl)-3H-imidazo[4,5-c]pyridin-4-one

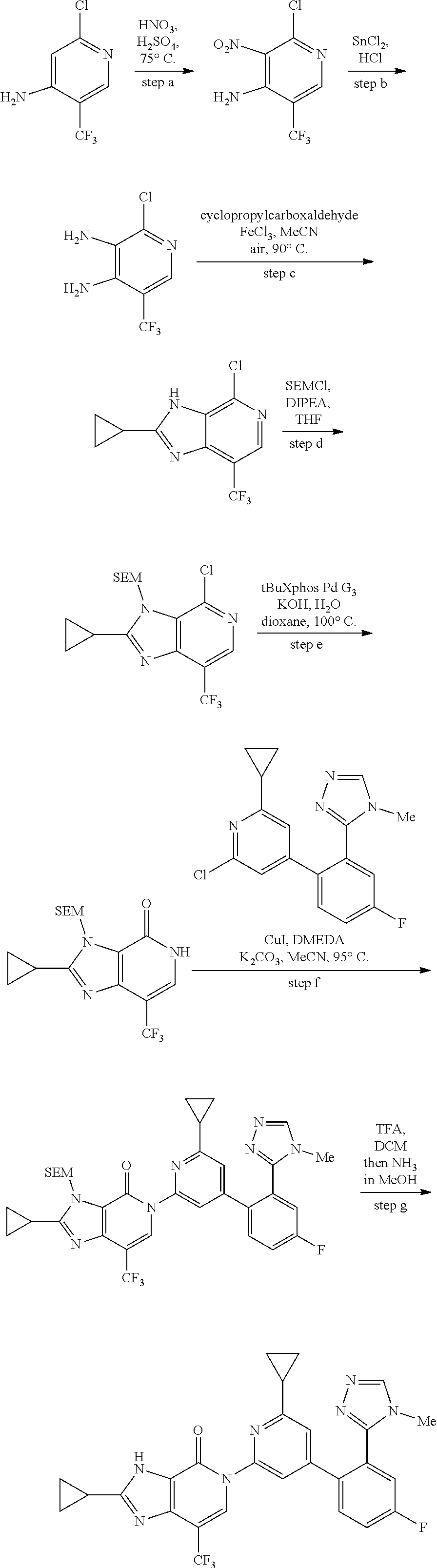

Step a: 2-Chloro-5-(trifluoromethyl)-4-pyridinamine (5.0 g, 25.5 mmol) was dissolved in H₂SO₄ (30 mL). Fuming nitric acid (10 mL) was added dropwise, and the resulting mixture was heated at 75° C. for 4 h, cooled to room temperature and carefully neutralized with aq. NaOH (2M) to pH˜8-9. The product was extracted with CHCl₃/iPrOH 3:1 mixture (3×50 mL). The combined extract was dried over Na₂SO₄ and concentrated under reduced pressure. The crude product was directly used for the next step without purification.

Step b: The product from step a (approx. 25.5 mmol) was dissolved in concentrated aq. HCl (30 mL), and the resulting solution was heated to 90° C. A solution of SnCl₂ (19.4 g, 102 mmol) in 15 mL conc. aq. HCl was added dropwise over 10 min. The resulting mixture was maintained at 75° C. for 1 h, then cooled to room temperature and neutralized to pH˜7 by slow addition of aq. NaOH (2M). The product was extracted with CHCl₃/iPrOH 3:1 mixture (3×50 mL), the combined organic extract was dried over Na₂SO₄ and concentrated to dryness under reduced pressure. The crude product was purified by column chromatography (SiO₂, 0-30% EtOAc gradient in hexanes) to afford 2-chloro-5-(trifluoromethyl)pyridine-3,4-diamine.

Step c: To a solution of the product from step b (211 mg, 1.0 mmol) in MeCN (5 mL, 0.2 M) was added cyclopropanecarboxaldehyde (0.11 mL, 1.5 mmol) and FeCl₃ (162 mg, 1.0 mmol). The resulting mixture was heated overnight at 90° C. under air atmosphere. The obtained mixture was cooled to room temperature and concentrated to dryness under reduced pressure. The crude residue was directly fractionated by column chromatography (SiO₂, 0-50% EtOAc gradient in hexanes) to afford 4-chloro-2-cyclopropyl-7-(trifluoromethyl)-3H-imidazo[4,5-c]pyridine.

Step d: To a solution of the product from step c (157 mg, 0.6 mmol) in THE (3 mL, 0.2 M) DIPEA (0.2 mL, 1.2 mmol) and SEM-Cl (0.16 mL, 0.9 mmol) were sequentially added. The resulting mixture was stirred at room temperature overnight, then diluted with EtOAc (20 mL) and water (20 mL). The organic phase was separated, and the aqueous phase was additionally extracted with EtOAc (2×20 mL). The combined organic extract was washed with water (50 mL), dried over Na₂SO₄, and concentrated to dryness under reduced pressure. The crude product was purified by column chromatography (SiO₂, 0-10% EtOAc gradient in hexanes) to yield 2-[[4-chloro-2-cyclopropyl-7-(trifluoromethyl)imidazo[4,5-c]pyridin-3-yl]methoxy]ethyl-trimethylsilane.

Step e: tBuXphos Pd G3 (127 mg, 0.16 mmol) and KOH (460 mg, 8.2 mmol) were added to the solution of product from step d (320 mg, 0.81 mmol) in dioxane (8 mL, 0.1 M), and the reaction mixture was refluxed overnight. The resulting solution was cooled to room temperature and extracted with EtOAc (3×20 mL). The combined organic extract was washed with water (30 mL), dried over Na₂SO₄ and concentrated to dryness under reduced pressure. The crude product was purified by column chromatography (SiO₂, 0-10% MeOH gradient in dichloromethane) to furnish 2-cyclopropyl-7-(trifluoromethyl)-3-(2-trimethylsilylethoxy-methyl)-5H-imidazo[4,5-c]pyridin-4-one.

Step f: To a mixture of the product from step e (37 mg, 0.1 mmol), 2-chloro-6-cyclopropyl-4-[4-fluoro-2-(4-methyl-1,2,4-triazol-3-yl)phenyl]pyridine (32.8 mg, 0.1 mmol, prepared according to example 28), DMEDA (21 μL, 0.2 mmol) and K₂CO₃ (41.4 mg, 0.3 mmol) in CH₃CN (2 mL, 0.05 M) was added CuI (19 mg, 0.1 mmol). The reaction mixture was sparged with nitrogen for 10 min and heated in a sealed vial at 95° C. overnight. The resulting suspension was cooled to 23° C. and diluted with EtOAc (10 mL) and sat. aq. NH₄Cl (5 mL). The organic phase was separated, and the aqueous layer was additionally extracted with EtOAc (5 mL). The combined organic phase was dried over Na₂SO₄ and concentrated to dryness under reduced pressure. The crude residue was purified by column chromatography (SiO₂, 0-10% EtOAc gradient in hexanes) to afford 2-cyclopropyl-5-[6-cyclopropyl-4-[4-fluoro-2-(4-methyl-1,2,4-triazol-3-yl)phenyl]pyridin-2-yl]-7-(trifluoromethyl)-3-(2-trimethylsilylethoxymethyl)imidazo[4,5-c]pyridin-4-one.

Step g: The product from step f (approx. 0.1 mmol) was dissolved in dichloromethane (1 mL). Trifluoroacetic acid (1 mL) was added, and the mixture was stirred at 23° C. for 1 h. All volatiles were removed under vacuum, and the crude material was redissolved in NH₃ in MeOH solution (7 M). After additional 30 min of stirring the solvent was evaporated under vacuum, and the crude residue was directly fractionated by preparative HPLC (C18 SiO₂, 10-90% CH₃CN in water with 0.1% formic acid) to afford the title compound. ¹H NMR (400 MHz, CDCl₃) 8.22 (s, 1H), 8.10 (d, J=7.4 Hz, 1H), 7.98 (s, 1H), 7.63-7.56 (m, 1H), 7.48 (t, J=7.7 Hz, 1H), 7.40 (t, J=7.7 Hz, 1H), 7.35 (s, 1H), 7.00 (s, 1H), 3.18 (s, 3H), 2.19 (t, J=6.6 Hz, 1H), 2.06-1.95 (m, 1H), 1.18 (d, J=4.7 Hz, 2H), 1.11 (dt, J=8.5, 3.1 Hz, 2H), 1.03 (d, J=8.1 Hz, 2H), 0.97 (d, J=3.8 Hz, 2H). ESI MS [M+H]+ for C₂₈H₂₃F₄N₆O, calcd 536.1, found 536.1.

### Example 40: 2-Cyclobutyl-5-[6-cyclopropyl-4-[4-fluoro-2-(4-methyl-1,2,4-triazol-3-yl)phenyl]pyridin-2-yl]-7-(trifluoromethyl)-3H-imidazo[4,5-c]pyridin-4-one

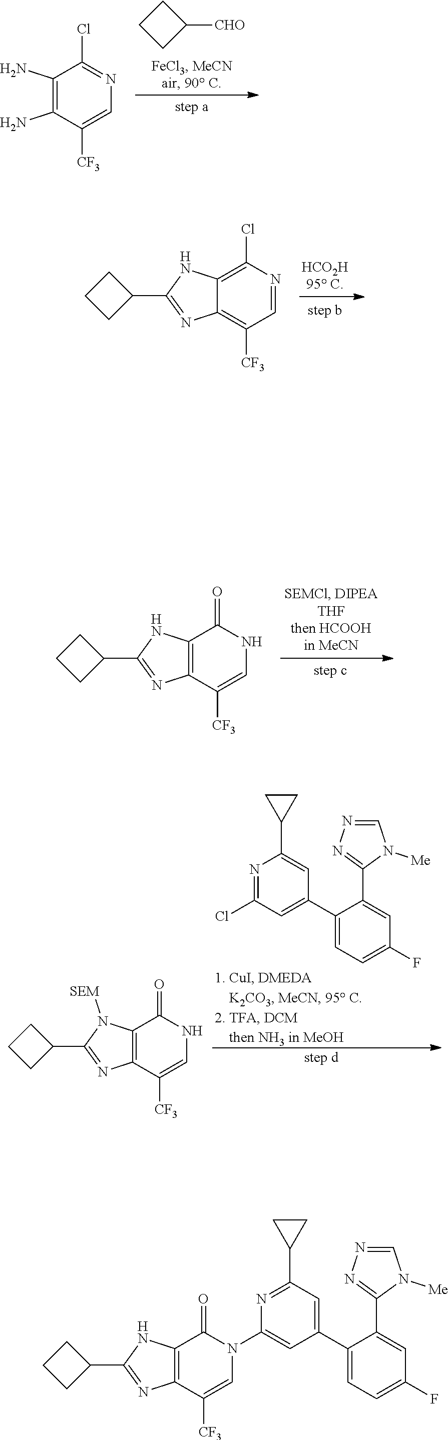

Step a: To a solution of 2-chloro-5-(trifluoromethyl)pyridine-3,4-diamine (211 mg, 1.0 mmol, prepared according to example 39) in CH₃CN (5 mL, 0.2 M) was added cyclobutanecarboxaldehyde (168 mg, 2.0 mmol) and FeCl₃ (162 mg, 1.0 mmol). The resulting mixture was heated overnight at 90° C. under air atmosphere. The obtained mixture was cooled to room temperature and concentrated to dryness under reduced pressure. The crude product was directly used for the next step without purification.

Step b: The product of step a (approx. 1.0 mmol) was dissolved in formic acid (2 mL), and the resulting solution was heated at 95° C. overnight. Upon cooling to room temperature, the solvent was removed under reduced pressure, and the crude product was purified by column chromatography (SiO₂, 0-100% EtOAc gradient in hexanes) to give the desired product.

Step c: The product from step b (367 mg, 1.4 mmol) was dissolved in THE (7 mL, 0.2 M), then DIPEA (1.2 mL, 7.0 mmol) and SEM-Cl (0.6 mL, 3.6 mmol) were added sequentially, and the mixture was stirred at 23° C. overnight. The reaction mixture was diluted with EtOAc (30 mL) and water (20 mL). The organic phase was separated, and the aqueous phase was additionally extracted with EtOAc (15 mL). The combined organic phase was dried over Na₂SO₄ and concentrated to dryness under reduced pressure. The crude residue was redissolved in 1% formic acid solution in CH₃CN (20 mL) and stirred at 23° C. for 1 h. Upon concentration under vacuum the crude product was directly purified by column chromatography (SiO₂, 0-10% EtOAc gradient in hexanes) to yield 2-cyclobutyl-7-(trifluoromethyl)-3-(2-trimethylsilylethoxymethyl)-5H-imidazo[4,5-c]pyridin-4-one.

Step d: This step was performed according to protocols described for example 39, steps f and g to afford the title product. ¹H NMR (400 MHz, CDCl₃) δ 8.24 (s, 1H), 8.00 (d, J=1.6 Hz, 1H), 7.59 (dd, J=9.3, 5.4 Hz, 1H), 7.42-7.31 (m, 3H), 7.02 (s, 1H), 3.78 (p, J=8.8 Hz, 1H), 3.18 (s, 3H), 2.58-2.25 (m, 4H), 2.13-1.94 (m, 2H), 1.86 (s, 2H), 1.03 (dt, J=8.2, 2.9 Hz, 2H), 0.98 (dt, J=5.3, 2.9 Hz, 2H). ESI MS [M+H]⁺ for C29H25F4N6O, calcd 550.2, found 550.1.

### Example 41: 5-{6-Cyclopropyl-4-[4-fluoro-2-(4-methyl-4H-1,2,4-triazol-3-yl)phenyl]-2-pyridyl}-2-(3-methoxypropyl)-7-(trifluoromethyl)-3,5-dihydro-1,3,5-triaza-4-indenone

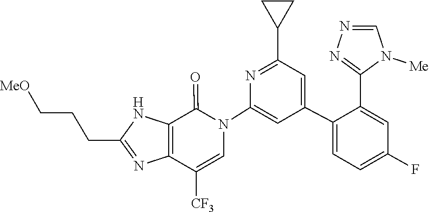 [SMILES: COCCCc1nc2c(C(F)(F)F)cn(-c3cc(-c4ccc(F)cc4-c4nncn4C)cc(C4CC4)n3)c(=O)c2[nH]1]

The title compound was prepared in a similar fashion to that described for example 40 using 4-methoxybutanal for step a. ¹H NMR (400 MHz, CDCl₃) δ 11.58 (s, 1H), 8.02 (d, J=1.6 Hz, 1H), 7.57 (dd, J=8.6, 5.3 Hz, 1H), 7.54-7.49 (m, 1H), 7.43 (s, 1H), 7.34 (d, J=8.2 Hz, 2H), 6.91 (s, 1H), 3.52 (t, J=5.6 Hz, 2H), 3.48 (d, J=5.4 Hz, 3H), 3.41 (s, 3H), 1.99-1.94 (m, 1H), 1.02-0.87 (m, 8H). +ESI MS [M+H]⁺ for C₂₈H₂₆F₄N₇O₂, calcd 568.2, found 568.1.

### Example 42: 2-(Cyclobutylmethyl)-5-{6-cyclopropyl-4-[4-fluoro-2-(4-methyl-4H-1,2,4-triazol-3-yl)phenyl]-2-pyridyl}-7-(trifluoromethyl)-3,5-dihydro-1,3,5-triaza-4-indenone

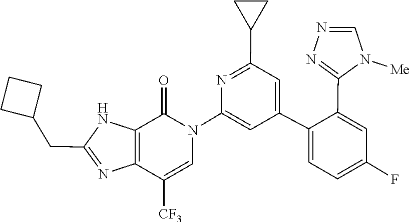 [SMILES: Cn1cnnc1-c1cc(F)ccc1-c1cc(C2CC2)nc(-n2cc(C(F)(F)F)c3nc(CC4CCC4)[nH]c3c2=O)c1]

The title compound was prepared in a similar fashion to that described for example 40 by using 2-cyclobutylacetaldehyde in step a. ¹H NMR (400 MHz, CDCl₃) δ 10.99 (s, 1H), 8.21 (s, 1H), 8.01 (s, 1H), 7.60 (dd, J=8.5, 5.4 Hz, 1H), 7.45-7.30 (m, 4H), 7.03 (d, J=1.3 Hz, 1H), 3.14 (s, 3H), 3.04 (d, J=7.7 Hz, 2H), 2.82-2.74 (m, 1H), 2.20-1.97 (m, 4H), 1.96-1.71 (m, 6H). ESI MS [M+H]⁺ for C₂₉H₂₆F₄N₇O, calcd 564.2, found 564.1.

### Example 43: 2-(Cyclohexylmethyl)-5-{6-cyclopropyl-4-[4-fluoro-2-(4-methyl-4H-1,2,4-triazol-3-yl)phenyl]-2-pyridyl}-7-(trifluoromethyl)-3,5-dihydro-1,3,5-triaza-4-indenone

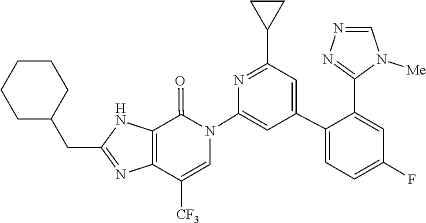 [SMILES: Cn1cnnc1-c1cc(F)ccc1-c1cc(C2CC2)nc(-n2cc(C(F)(F)F)c3nc(CC4CCCCC4)[nH]c3c2=O)c1]

The title compound was prepared in a similar fashion to that described for example 40 by using 2-cyclohexylacetaldehyde in step a. ¹H NMR (400 MHz, CDCl₃) δ 12.05 (s, 1H), 8.40 (s, 1H), 7.95 (d, J=1.5 Hz, 1H), 7.62 (dd, J=8.5, 5.3 Hz, 1H), 7.42-7.36 (m, 2H), 7.28 (d, J=1.4 Hz, 1H), 7.11 (d, J=1.4 Hz, 1H), 3.15 (s, 3H), 2.76 (d, J=7.3 Hz, 2H), 2.05 (d, J=2.3 Hz, 1H), 1.85 (s, 4H), 1.20-0.97 (m, 8H), 0.91 (q, J=12.5, 11.9 Hz, 3H). ESI MS [M+H]⁺ for C₃₁H₃₀F₄N₇O, calcd 592.2, found 592.2.

### Example 44: 2-[(2R)-1-Acetylpyrrolidin-2-yl]-5-[6-cyclopropyl-4-[4-fluoro-2-(4-methyl-1,2,4-triazol-3-yl)phenyl]pyridin-2-yl]-7-(trifluoromethyl)-3-(2-trimethylsilylethoxymethyl)imidazo[4,5-c]pyridin-4-one

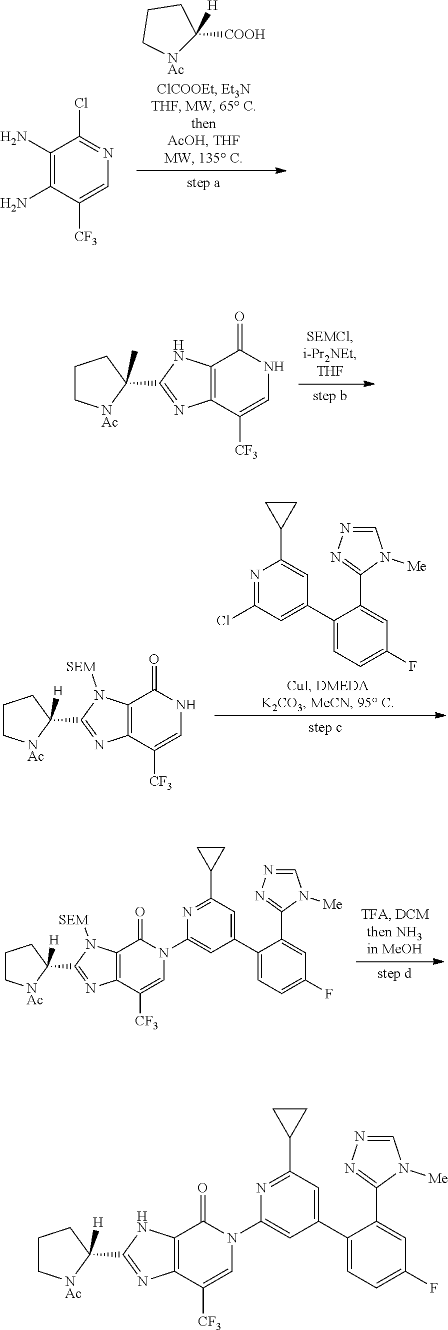

Step a: To a solution of (2R)-1-acetylpyrrolidine-2-carboxylic acid (516 mg, 2.4 mmol) in THF (5 mL) was added Et₃N (0.67 mL, 4.8 mmol) and ClCOOEt (0.23 mL, 2.4 mmol). The resulting mixture was stirred at room temperature for 1 h before 2-chloro-5-(trifluoromethyl)pyridine-3,4-diamine (211 mg, 1.0 mmol, prepared according to example 39) was added. The reaction mixture was stirred at 65° C. under microwave irradiation for 18 h, then cooled to room temperature and concentrated to dryness. The crude intermediate was purified by column chromatography (SiO₂, 0-20% EtOAc gradient in hexanes). Then it was dissolved in THE (5 mL) and AcOH (5 mL) and stirred at 135° C. under microwave irradiation for 4 h. The resulting solution was cooled to 23° C., and the solvent was evaporated to dryness. The crude product was purified by column chromatography (SiO₂, 0-20% MeOH gradient in dichloromethane) to afford 2-[(2R)-1-acetylpyrrolidin-2-yl]-7-(trifluoromethyl)-3,5-dihydroimidazo[4,5-c]pyridin-4-one.

Steps b, c, and d were performed according to protocols described for example 40 to furnish the title compound. ¹H NMR (400 MHz, CDCl₃) δ 8.12 (s, 1H), 8.06 (d, J=1.6 Hz, 1H), 7.55 (s, 1H), 7.47 (s, 1H), 7.40 (dd, J=8.6, 2.7 Hz, 1H), 7.35 (dd, J=8.3, 2.7 Hz, 1H), 6.84 (s, 1H), 5.32 (s, 1H), 3.82-3.41 (m, 2H), 3.18 (s, 3H), 2.32 (s, 1H), 2.15 (s, 3H), 1.96 (s, 1H), 1.64 (s, 3H), 0.99 (d, J=7.1 Hz, 2H), 0.91 (s, 2H). ESI MS [M+H]⁺ for C₃₁H₂₈F₄N₇O₂, calcd 607.2, found 607.1.

### Example 45: 2-[(2S)-1-acetylpyrrolidin-2-yl]-5-[6-cyclopropyl-4-[4-fluoro-2-(4-methyl-1,2,4-triazol-3-yl)phenyl]pyridin-2-yl]-7-(trifluoromethyl)-3H-imidazo[4,5-c]pyridin-4-one

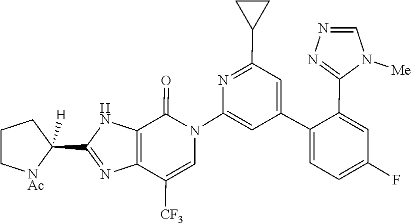

The title compound was prepared in similar fashion to that described for example 44 using (2S)-1-acetylpyrrolidine-2-carboxylic acid on the first step. ¹H NMR (400 MHz, CDCl₃) δ 8.12 (s, 1H), 8.06 (d, J=1.6 Hz, 1H), 7.55 (s, 1H), 7.47 (s, 1H), 7.40 (dd, J=8.6, 2.7 Hz, 1H), 7.35 (dd, J=8.3, 2.7 Hz, 1H), 6.84 (s, 1H), 5.32 (s, 1H), 3.82-3.41 (m, 2H), 3.18 (s, 3H), 2.32 (s, 1H), 2.15 (s, 3H), 1.96 (s, 1H), 1.64 (s, 3H), 0.99 (d, J=7.1 Hz, 2H), 0.91 (s, 2H). ESI MS [M+H]⁺ for C₃₁H₂₈F₄N₇O₂, calcd 607.2, found 607.1.

### Example 46: 2-[(1R)-1-cyclopropylethyl]-5-[6-cyclopropyl-4-[4-fluoro-2-(4-methyl-1,2,4-triazol-3-yl)phenyl]pyridin-2-yl]-7-(trifluoromethyl)-3H-imidazo[4,5-c]pyridin-4-one

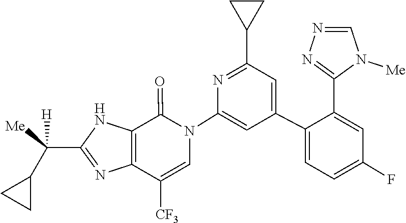 [SMILES: C[C@@H](c1nc2c(C(F)(F)F)cn(-c3cc(-c4ccc(F)cc4-c4nncn4C)cc(C4CC4)n3)c(=O)c2[nH]1)C1CC1]

The title compound was prepared in similar fashion to that described for example 44 using (2R)-2-cyclopropylpropanoic acid on the first step. ¹H NMR (400 MHz, CDCl₃) δ 8.16 (s, 1H), 8.03 (s, 1H), 7.58 (dd, J=8.5, 5.3 Hz, 1H), 7.38 (ddd, J=15.3, 7.7, 2.7 Hz, 3H), 6.99 (d, J=4.2 Hz, 1H), 3.16 (s, 3H), 2.46 (s, 1H), 2.01 (s, 1H), 1.50 (d, J=7.1 Hz, 3H), 1.11-1.00 (m, 3H), 0.97 (s, 2H), 0.63 (d, J=23.4 Hz, 2H), 0.34 (dq, J=11.0, 6.1, 5.4 Hz, 2H). ESI MS [M+H]⁺ for C₃₀H₂₇F₄N₆O, calcd 564.2, found 564.1.

### Example 47: 2-[2-(2-{2-[(S)-Tetrahydro-3-furyl]-4-oxo-7-(trifluoromethyl)-3,5-dihydro-1,3,5-triaza-5-indenyl}-6-cyclopropyl-4-pyridyl)-5-fluorophenyl]-1-methyl-4-imidazolecarbonitrile

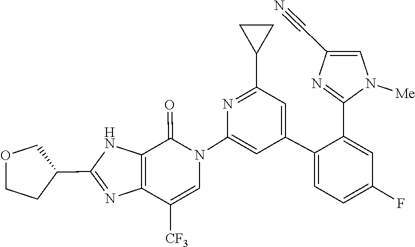 [SMILES: Cn1cc(C#N)nc1-c1cc(F)ccc1-c1cc(C2CC2)nc(-n2cc(C(F)(F)F)c3nc([C@@H]4CCOC4)[nH]c3c2=O)c1]

The title compound was prepared in a similar fashion to that described for example 44 using (S)-tetrahydrofuran-3-carboxylic in step a and 2-[2-(2-chloro-6-cyclopropylpyridin-4-yl)-5-fluorophenyl]-1H-imidazole-4-carbonitrile (prepared according to example 107) for step c. ¹H NMR (400 MHz, CDCl₃) δ 11.34 (s, 1H), 8.04 (q, J=1.4 Hz, 1H), 7.57-7.51 (m, 1H), 7.43-7.37 (m, 2H), 7.33 (td, J=7.7, 2.4 Hz, 2H), 6.89 (d, J=1.4 Hz, 1H), 4.04-3.92 (m, 4H), 3.85-3.72 (m, 2H), 3.15 (s, 3H), 2.37 (dtd, J=12.8, 8.4, 5.5 Hz, 1H), 2.27-2.12 (m, 1H), 1.98 (dt, J=8.1, 4.8 Hz, 1H), 1.09-0.84 (m, 5H). ESI MS [M+H]⁺ for C₃₀H₂₆F₄N₇O₂, calcd 590.2, found 590.2.

### Example 48: 2-[2-(2-{2-[(R)-Tetrahydro-3-furyl]-4-oxo-7-(trifluoromethyl)-3,5-dihydro-1,3,5-triaza-5-indenyl}-6-cyclopropyl-4-pyridyl)-5-fluorophenyl]-1-methyl-4-imidazolecarbonitrile

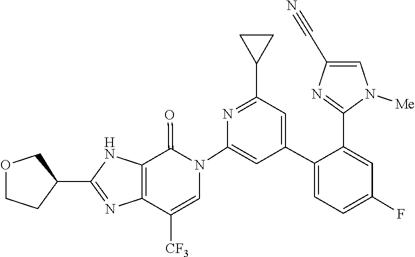 [SMILES: Cn1cc(C#N)nc1-c1cc(F)ccc1-c1cc(C2CC2)nc(-n2cc(C(F)(F)F)c3nc([C@H]4CCOC4)[nH]c3c2=O)c1]

The title compound was prepared in a similar fashion to that described for example 44 using (R)-tetrahydrofuran-3-carboxylic in step a and 2-[2-(2-chloro-6-cyclopropylpyridin-4-yl)-5-fluorophenyl]-1H-imidazole-4-carbonitrile (prepared according to example 107) for step c. ¹H NMR (400 MHz, CDCl₃) δ 11.34 (s, 1H), 8.04 (q, J=1.4 Hz, 1H), 7.57-7.51 (m, 1H), 7.43-7.37 (m, 2H), 7.33 (td, J=7.7, 2.4 Hz, 2H), 6.89 (d, J=1.4 Hz, 1H), 4.04-3.92 (m, 4H), 3.85-3.72 (m, 2H), 3.15 (s, 3H), 2.37 (dtd, J=12.8, 8.4, 5.5 Hz, 1H), 2.27-2.12 (m, 1H), 1.98 (dt, J=8.1, 4.8 Hz, 1H), 1.09-0.84 (m, 5H). ESI MS [M+H]⁺ for C₃₀H₂₆F₄N₇O₂, calcd 590.2, found 590.2.

### Example 49: 2-{[(S)-2-methoxypropylamino]methyl}-5-{6-cyclopropyl-4-[4-fluoro-2-(5-methyl-1,3-oxazol-4-yl)phenyl]-2-pyridyl}-7-(trifluoromethyl)-3,5-dihydro-1,3,5-triaza-4-indenone

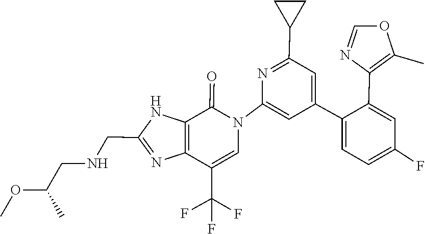

The title compound was prepared in a similar fashion to that described for example 50 using (S)-2-methoxypropylamine for in step f and 4-[2-(2-chloro-6-cyclopropylpyridin-4-yl)-5-fluorophenyl]-5-methyl-1,3-oxazole (prepared according to example 78) for step g. ¹H NMR (400 MHz, CDCl₃) δ 8.04 (q, J=1.4 Hz, 1H), 7.77 (s, 1H), 7.44 (dd, J=8.6, 5.6 Hz, 1H), 7.41 (d, J=1.4 Hz, 1H), 7.26 (d, J=2.6 Hz, 1H), 7.16 (td, J=8.2, 2.7 Hz, 1H), 6.99 (d, J=1.4 Hz, 1H), 4.17 (d, J=1.3 Hz, 2H), 3.52-3.46 (m, 1H), 3.40 (s, 3H), 2.78 (d, J=3.0 Hz, 1H), 2.66 (dd, J=12.8, 8.2 Hz, 2H), 2.01-1.97 (m, 1H), 1.95 (s, 3H), 1.13 (dd, J=6.2, 2.1 Hz, 3H), 1.02-0.90 (m, 5H). ESI MS [M+H]⁺ for C₃₀H₂₉F₄N₆O₃, calcd 597.2, found 597.2.

### Example 50: 5-[6-cyclopropyl-4-[4-fluoro-2-(4-methyl-1,2,4-triazol-3-yl)phenyl]pyridin-2-yl]-2-[[(3S)-3-methylpiperidin-1-yl]methyl]-7-(trifluoromethyl)-3H-imidazo[4,5-c]pyridin-4-one

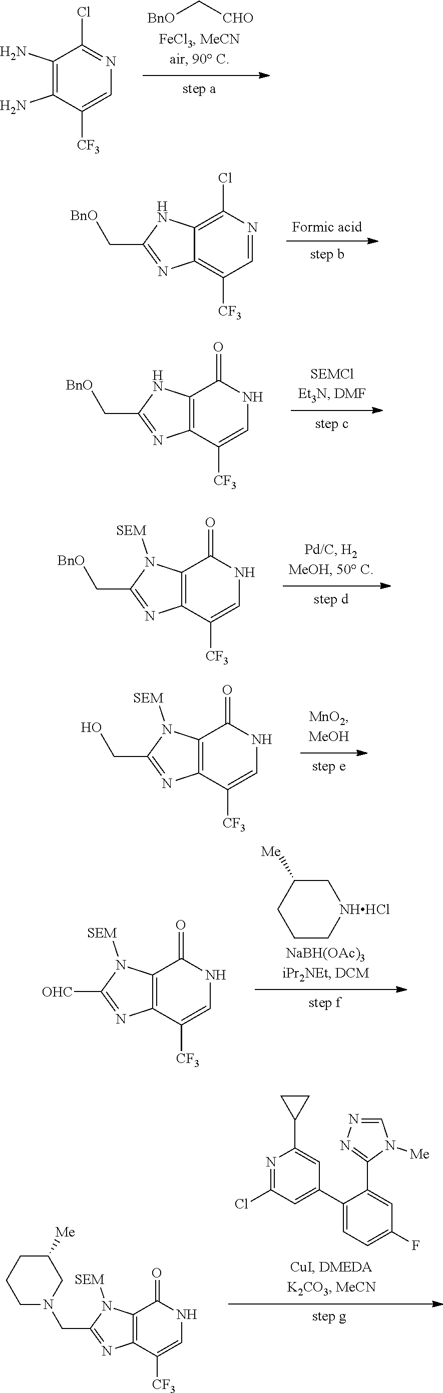

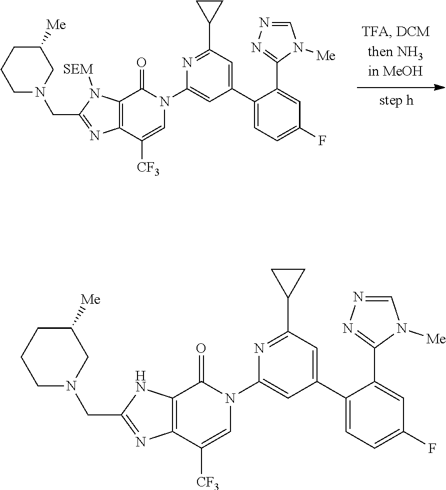 [SMILES: C[C@H]1CCCN(Cc2nc3c(C(F)(F)F)cn(-c4cc(-c5ccc(F)cc5-c5nncn5C)cc(C5CC5)n4)c(=O)c3n2COCC[Si](C)(C)C)C1 ; C[C@H]1CCCN(Cc2nc3c(C(F)(F)F)cn(-c4cc(-c5ccc(F)cc5-c5nncn5C)cc(C5CC5)n4)c(=O)c3[nH]2)C1]

Step a: To a solution of 2-chloro-5-(trifluoromethyl)pyridine-3,4-diamine (2.96 g, 14.0 mmol, prepared according to example 39) in CH₃CN (80 mL) was added 2-phenylmethoxyacetaldehyde (3.15 g, 21.0 mmol) and FeCl₃ (453 mg, 2.8 mmol). The resulting mixture was heated overnight at 90° C. under air atmosphere. The obtained mixture was cooled to room temperature and concentrated to dryness under reduced pressure. The crude residue was directly fractionated by column chromatography (SiO₂, 0-40% EtOAc gradient in hexanes) to afford 4-chloro-2-(phenylmethoxymethyl)-7-(trifluoromethyl)-3H-imidazo[4,5-c]pyridine.

Step b: The product of step a (163 mg, 0.48 mmol) was dissolved in formic acid (5 mL, 0.1 M), and the resulting solution was stirred at 90° C. overnight. The obtained mixture was cooled to room temperature and formic acid was evaporated under reduced pressure. Purification by column chromatography (SiO₂, 0-10% MeOH gradient in dichloromethane) furnished 2-(phenylmethoxymethyl)-7-(trifluoromethyl)-3,5-dihydroimidazo[4,5-c]pyridin-4-one.

Step c: The product from step b (78.6 mg, 0.24 mmol) was dissolved in DMF (1.1 mL) followed by the addition of Et₃N (0.1 mL mg, 0.72 mmol) and SEM-Cl (50 mg, 0.3 mmol). The reaction mixture was stirred at room temperature overnight followed by partitioning between EtOAc (15 mL) and water (15 mL). The organic phase was separated, and the aqueous layer was additionally extracted with EtOAc (2×10 mL). The combined organic phase was washed with water (2×20 mL), dried over Na₂SO₄ and concentrated to dryness under reduced pressure. The crude material was fractionated by column chromatography (SiO₂, 0-100% EtOAc gradient in hexanes) to yield 2-(phenylmethoxymethyl)-7-(trifluoromethyl)-3-(2-trimethylsilylethoxymethyl)-5H-imidazo[4,5-c]pyridin-4-one.

Step d: To a solution of the product from step c (246 mg, 0.54 mmol) in methanol (5.4 mL, 0.1 M) palladium on carbon (246 mg, 10 wt % Pd) was added. The mixture was placed in sealed vial, degassed, and backfilled with hydrogen. The resulting mixture was vigorously stirred at 50° C. under hydrogen atmosphere overnight. The obtained suspension was filtered through a pad of Celite® that was additionally washed with methanol (5 mL). The filtrate was concentrated to dryness under vacuum to afford crude product that was used for the next step without purification.

Step e: The crude alcohol product from step d (approx. 0.54 mmol) was dissolved in MeOH (2.7 mL, 0.2 M) followed by the addition of MnO₂ (139 mg, 1.6 mmol). The resulting mixture was stirred at 50° C. overnight. The obtained solution was passed through a Celite® pad upon cooling to 23° C. The precipitate was additionally washed with MeOH (5 mL), and the combined filtrate was concentrated to dryness under vacuum. The crude product was purified by column chromatography (SiO₂, 0-10% MeOH gradient in dichloromethane) to afford 4-oxo-7-(trifluoromethyl)-3-(2-trimethylsilylethoxymethyl)-5H-imidazo[4,5-c]pyridine-2-carbaldehyde.

Step f: The product from step e (48 mg, 0.13 mmol) was dissolved in dichloromethane (1 mL), and (3S)-3-methyl-piperidine hydrochloride (26.3 mg, 0.2 mmol) and i-Pr₂NEt (34 mg, 0.26 mmol) were added. The reaction was stirred at room temperature for 30 mins before NaBH(OAc)₃ (41 mg, 0.2 mmol) was added. The resulting reaction was stirred for another hour, then diluted with water (5 mL) and dichloromethane (5 mL). The organic phase was separated, and the aqueous phase was additionally extracted with dichloromethane (5 mL). The combined organic phase was dried over Na₂SO₄, and the solvent was removed in vacuum. The crude product was purified by column chromatography (SiO₂, 0-10% MeOH gradient in dichloromethane) to afford 2-[[(3S)-3-methylpiperidin-1-yl]methyl]-7-(trifluoromethyl)-3-(2-trimethylsilylethoxymethyl)-5H-imidazo[4,5-c]pyridin-4-one.

Steps g, h: Steps g and h were performed according to corresponding protocols described for example 39 for steps f, g. ¹H NMR (400 MHz, CDCl₃) δ 8.11 (s, 1H), 8.08 (d, J=1.5 Hz, 1H), 7.58 (dd, J=8.6, 5.4 Hz, 1H), 7.49 (d, J=1.4 Hz, 1H), 7.45-7.32 (m, 2H), 6.89 (d, J=1.4 Hz, 1H), 3.92 (s, 2H), 3.18 (s, 3H), 3.07-2.78 (m, 2H), 2.24 (s, 1H), 2.07-1.87 (m, 2H), 1.84-1.62 (m, 5H), 1.01 (dt, J=8.1, 3.0 Hz, 2H), 0.96-0.91 (m, 2H), 0.89 (d, J=6.4 Hz, 3H). ESI MS [M+H]⁺ for C₃₂H₃₂F₄N₇O, calcd 607.2, found 607.2.

### Example 51: 7-cyclopropyl-5-[6-cyclopropyl-4-[4-fluoro-2-(1-methylimidazol-2-yl)phenyl]pyridin-2-yl]-2-[[(3S)-3-methylpiperidin-1-yl]methyl]-3H-imidazo[4,5-c]pyridin-4-one

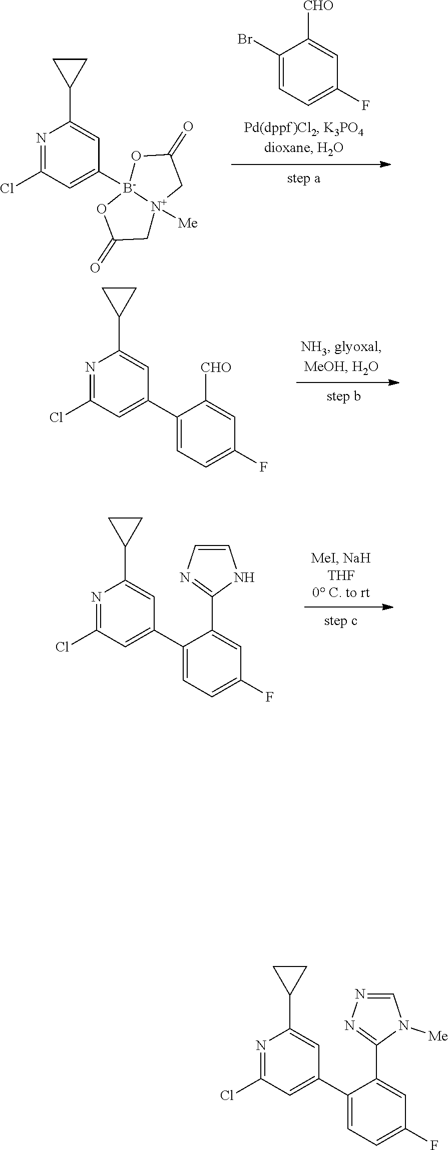

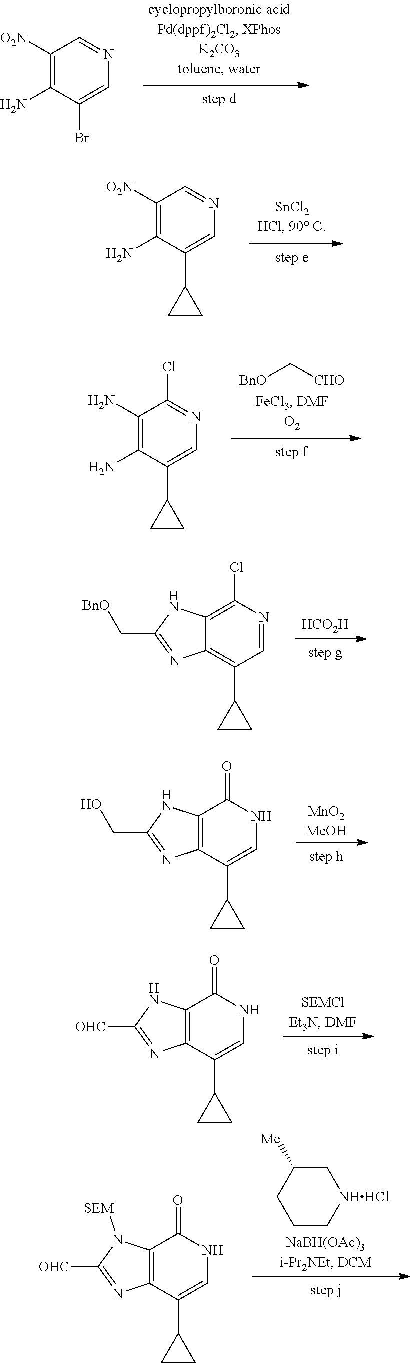

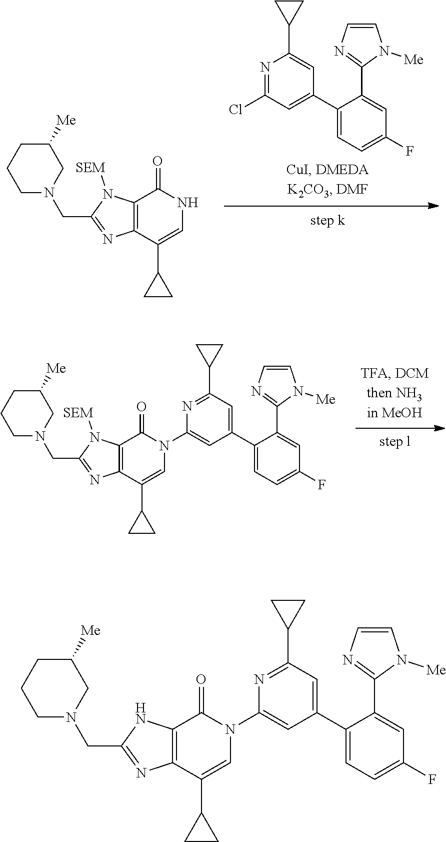 [SMILES: C[C@H]1CCCN(Cc2nc3c(C4CC4)c[nH]c(=O)c3n2COCC[Si](C)(C)C)C1 ; Cn1ccnc1-c1cc(F)ccc1-c1cc(Cl)nc(C2CC2)c1 ; C[C@H]1CCCN(Cc2nc3c(C4CC4)cn(-c4cc(-c5ccc(F)cc5-c5nccn5C)cc(C5CC5)n4)c(=O)c3n2COCC[Si](C)(C)C)C1 ; C[C@H]1CCCN(Cc2nc3c(C4CC4)cn(-c4cc(-c5ccc(F)cc5-c5nccn5C)cc(C5CC5)n4)c(=O)c3[nH]2)C1]

Step a: To a mixture of 1-(2-chloro-6-cyclopropylpyridin-4-yl)-5-methyl-2,8-dioxa-5-azonia-1-boranuidabicyclo[3.3.0]octane-3,7-dione (2.15 g, 7.0 mmol, 1.0 equiv., prepared according to example 18) and 2-bromo-5-fluorobenzaldehyde (1.42 g, 7.0 mmol, 1.0 equiv.) in dioxane/water (50 mL, 4:1 v/v) was added Pd(dppf)Cl₂ (512 mg, 0.70 mmol, 10 mol %) and K₃PO₄ (3.0 g, 14 mmol, 2.0 equiv.). The resulting mixture was heated at 100° C. for 2 h, cooled to room temperature and diluted with EtOAc (50 mL) and water (20 mL). The organic phase was separated, and the aqueous layer was extracted with EtOAc (20 mL). The combined organic phase was dried over Na₂SO₄, concentrated, and the crude residue was purified by column chromatography (SiO₂, EtOAc in hexanes, 0 to 15%) to afford 2-(2-chloro-6-cyclopropylpyridin-4-yl)-5-fluorobenzaldehyde.

Step b: To the product from step a (1.93 g, 7.0 mmol, 1.0 equiv.) in MeOH (20 mL) was added 7M NH₃ in methanol (10 mL, 70 mmol, 10 equiv.) and glyoxal aqueous solution (5.6 mL, 49 mmol, 7.0 equiv., 40 wt %). The resulting mixture was stirred at room temperature for 60 h, and then all volatiles were removed under vacuum. The crude residue was purified by column chromatography (SiO₂, 0 to 50% EtOAc gradient in hexanes) to afford 2-chloro-6-cyclopropyl-4-[4-fluoro-2-(1H-imidazol-2-yl)phenyl]pyridine.

Step c: To a solution of the product from step b (1.17 g, 3.7 mmol, 1.0 equiv.) in THE (30 mL) was added NaH (164 mg, 4.1 mmol, 1.1 equiv., 60 wt % in mineral oil) at 0° C. The resulting mixture was stirred at this temperature for 10 min before the addition of MeI (0.47 mL, 7.5 mmol, 2.0 equiv.). The reaction mixture was then stirred at room temperature for 1 h before quenched with H₂O (5 mL). The reaction mixture was diluted with EtOAc (40 mL), the organic phase was separated, and the aqueous layer was extracted with EtOAc (2×25 mL). The combined organic phase was dried over Na₂SO₄, concentrated, and the crude residue was purified by column chromatography (SiO₂, EtOAc in hexanes, 0 to 50%) to afford 2-chloro-6-cyclopropyl-4-[4-fluoro-2-(1-methylimidazol-2-yl)phenyl]pyridine.

Step d: 3-Bromo-5-nitro-4-pyridinamine (218 mg, 1.0 mmol, 1 equiv.), cyclopropylboronic acid (129 mg, 1.5 mmol, 1.5 equiv.), Xphos (38.0 mg, 0.08 mmol, 0.08 equiv.) and K₂CO₃ (414 mg, 3.0 mmol, 3 equiv.) were added to a mixture of toluene (8 mL) and water (2 mL). The reaction was degassed by three vacuum/backfill with nitrogen cycles followed by addition of Pd(dppf)Cl₂ (36.6 mg, 0.05 mmol, 0.05 equiv.). The resulting mixture was stirred at 95° C. overnight. Once cooled to 23° C. the biphasic solution was diluted with water (10 mL) and EtOAc (30 mL). The organic layer was separated, and the aqueous layer was additionally extracted with EtOAc (2×15 mL). The combined organic extract was dried over Na₂SO₄ and concentrated to dryness under reduced pressure. Purification of the crude material by column chromatography (SiO₂, 0-80% EtOAc gradient in hexanes) afforded 3-cyclopropyl-5-nitropyridin-4-amine.

Step e: To the solution of the product from step d (156 mg, 0.87 mmol, 1 equiv.) in concentrated aq. HCl (3 mL), a preheated to 90° C. solution of SnCl₂ (0.66 g, 3.5 mmol) in concentrated aq. HCl (1.5 mL) was added dropwise over 10 min. The reaction vial was sealed and heated at 130° C. for 3 h. The obtained mixture was allowed to cool to room temperature and carefully neutralized with aq. NaOH (2M) to pH˜7. The product was extracted with CHCl₃/i-PrOH mixture (2×20 mL, 3:1 v/v), the combined organic phase was dried over Na₂SO₄ and the volatiles were removed under vacuum. The crude residue was purified by column chromatography (SiO₂, 40-100% EtOAc gradient in hexanes) to furnish 2-chloro-5-cyclopropylpyridine-3,4-diamine.

Steps f-l: Steps f-j and steps k-l were performed in an analogous manner to that described in example 50, steps a, b, e, c and f, and example 39, steps f and g, respectively, to afford the title compound. ¹H NMR (400 MHz, CDCl₃) δ 7.58 (s, 1H), 7.53 (dd, J=8.7, 5.4 Hz, 2H), 7.39-7.32 (m, 3H), 7.13 (s, 1H), 6.83 (s, 1H), 6.53 (s, 1H), 4.04 (s, 2H), 3.10 (s, 3H), 3.01 (s, 3H), 2.00 (s, 5H), 1.91 (dq, J=8.5, 4.9, 4.2 Hz, 1H), 1.03-0.94 (m, 1H), 0.94-0.88 (m, 3H), 0.83 (q, J=3.8 Hz, 3H), 0.73 (s, 3H). ESI MS [M+H]⁺ for C₃₅H₃₈FN₆O, calcd 578.3, found 578.2.

### Example 52: 2-{[(S)-3-methyl-1-piperidyl]methyl}-4-cyclopropyl-6-{6-cyclopropyl-4-[4-fluoro-2-(1-methyl-2-imidazolyl)phenyl]-2-pyridyl}-1,6-dihydro-1,6-diaza-7-indenone

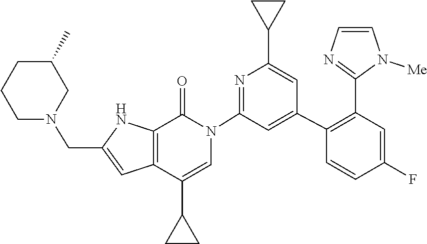 [SMILES: C[C@H]1CCCN(Cc2cc3c(C4CC4)cn(-c4cc(-c5ccc(F)cc5-c5nccn5C)cc(C5CC5)n4)c(=O)c3[nH]2)C1]

The title compound was prepared in a similar fashion to the example 2 (steps b, c) starting from 2-chloro-6-cyclopropyl-4-[4-fluoro-2-(1-methylimidazol-2-yl)phenyl]pyridine (prepared according to example 51) and 4-cyclopropyl-2-[[(3S)-3-methylpiperidin-1-yl]methyl]-1-(2-trimethylsilylethoxymethyl)-6H-pyrrolo[2,3-c]pyridin-7-one (prepared according to example 1). ¹H NMR (400 MHz, CDCl₃) δ 9.58 (s, 1H), 7.67 (d, J=1.3 Hz, 1H), 7.54 (dd, J=8.6, 5.5 Hz, 1H), 7.35 (dd, J=8.9, 2.7 Hz, 1H), 7.27-7.19 (m, 2H), 7.14 (d, J=1.2 Hz, 1H), 6.82 (d, J=1.2 Hz, 1H), 6.46 (d, J=1.3 Hz, 1H), 6.36 (s, 1H), 3.60 (d, J=3.5 Hz, 2H), 3.08 (s, 3H), 2.77 (dd, J=18.3, 9.2 Hz, 2H), 2.01-1.82 (m, 4H), 1.80-1.48 (m, 4H), 0.93-0.75 (m, 10H), 0.74-0.61 (m, 2H). ESI MS [M+H]⁺ for C₃₅H₃₇FN₆O, calcd 577.3, found 577.2.

### Example 53: 4-cyclopropyl-6-{6-cyclopropyl-4-[4-fluoro-2-(1-methyl-2-imidazolyl)phenyl]-2-pyridyl}-2-{[1-(hydroxymethyl)cyclobutylamino]methyl}-1,6-dihydro-1,6-diaza-7-indenone

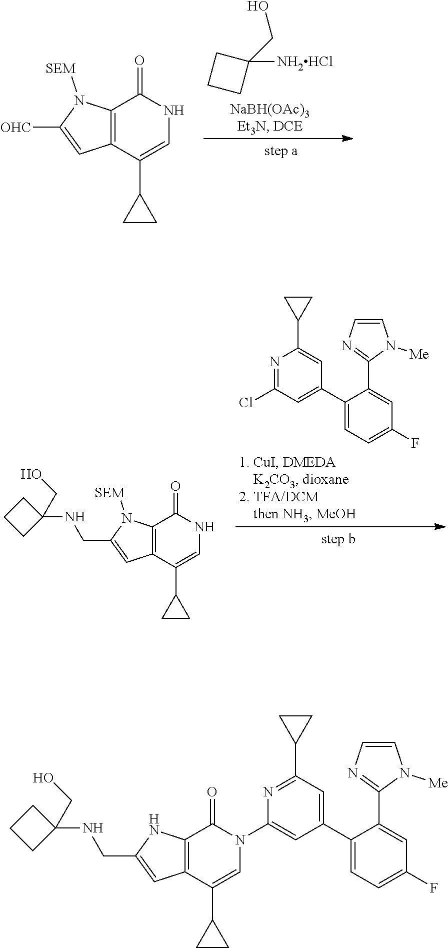

Step a: To a mixture of 4-cyclopropyl-7-oxo-1-(2-trimethylsilylethoxymethyl)-6H-pyrrolo[2,3-c]pyridine-2-carbaldehyde (166 mg, 0.50 mmol, 1.0 equiv., prepared according to example 1) and (1-aminocyclobutyl)methanol hydrochloride (68.8 mg, 0.50 mmol, 1.0 equiv.) in 1,2-dichloroethane (3.0 mL) was added Et₃N (0.14 mL, 101 mg, 1.0 mmol, 2.0 equiv.) at room temperature. The resulting mixture was stirred for 30 min before the addition of NaBH(OAc)₃ (159 mg, 0.75 mmol, 1.5 equiv.). The reaction mixture was then stirred at room temperature overnight, then diluted with water (10 mL) and dichloromethane (10 mL). The organic phase was separated, and the aqueous layer was extracted with dichloromethane (2×5 mL). The combined organic phase was dried over Na₂SO₄, concentrated, and the crude residue was purified by column chromatography (SiO₂, 0 to 10% MeOH in dichloromethane) to afford 4-cyclopropyl-2-[[[1-(hydroxymethyl)cyclobutyl]amino]methyl]-1-(2-trimethyl-silylethoxymethyl)-6H-pyrrolo[2,3-c]pyridin-7-one.

Step b: The reaction was performed in a similar fashion to that described for example 2 (steps b, c) starting from 2-chloro-6-cyclopropyl-4-[4-fluoro-2-(1-methylimidazol-2-yl)phenyl]pyridine (prepared according to example 51) to afford the title compound. ¹H NMR (400 MHz, CDCl₃) δ 7.60-7.52 (m, 2H), 7.34 (dd, J=8.9, 2.7 Hz, 1H), 7.31-7.21 (m, 1H), 7.14 (s, 1H), 7.12 (d, J=1.2 Hz, 1H), 6.84 (d, J=1.2 Hz, 1H), 6.57 (s, 1H), 6.27 (s, 1H), 3.96 (s, 2H), 3.69 (s, 2H), 3.12 (s, 3H), 2.16-2.07 (m, 2H), 2.01-1.68 (m, 6H), 1.01-0.91 (m, 2H), 0.92-0.84 (m, 4H), 0.75-0.55 (m, 2H). ESI MS [M+H]⁺ for C₃₄H₃₅FN₆O₂, calcd 579.3, found 579.2.

### Example 54: 2-{[(R)-2-methyl-4-morpholinyl]methyl}-4-cyclopropyl-6-{6-cyclopropyl-4-[4-fluoro-2-(1-methyl-2-imidazolyl)phenyl]-2-pyridyl}-1,6-dihydro-1,6-diaza-7-indenone

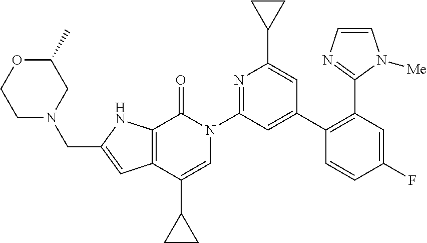 [SMILES: C[C@@H]1CN(Cc2cc3c(C4CC4)cn(-c4cc(-c5ccc(F)cc5-c5nccn5C)cc(C5CC5)n4)c(=O)c3[nH]2)CCO1]

The title compound was prepared in a similar fashion to that described for example 53 using (2R)-2-methylmorpholine hydrochloride for reductive amination step. ¹H NMR (400 MHz, CDCl₃) δ 9.53 (s, 1H), 7.64 (d, J=1.4 Hz, 1H), 7.54 (dd, J=8.6, 5.5 Hz, 1H), 7.35 (dd, J=8.9, 2.7 Hz, 1H), 7.28-7.22 (m, 1H), 7.23 (d, J=1.3 Hz, 1H), 7.14 (d, J=1.2 Hz, 1H), 6.82 (d, J=1.2 Hz, 1H), 6.48 (d, J=1.4 Hz, 1H), 6.39 (d, J=2.2 Hz, 1H), 3.93-3.79 (m, 1H), 3.75-3.57 (m, 4H), 3.08 (s, 3H), 2.79-2.61 (m, 2H), 2.23 (td, J=11.3, 3.3 Hz, 1H), 1.99-1.87 (m, 3H), 1.12 (d, J=6.3 Hz, 3H), 1.00-0.76 (m, 6H), 0.73-0.62 (m, 2H). ESI MS [M+H]⁺ for C₃₄H₃₅FN₆O₂, calcd 579.3, found 579.2.

### Example 55: 4-cyclopropyl-6-{6-cyclopropyl-4-[4-fluoro-2-(1-methyl-2-imidazolyl)phenyl]-2-pyridyl}-2-[(1-methylcyclobutylamino)methyl]-1,6-dihydro-1,6-diaza-7-indenone

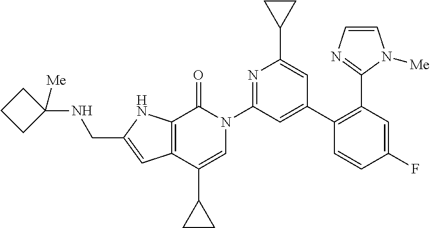 [SMILES: Cn1ccnc1-c1cc(F)ccc1-c1cc(C2CC2)nc(-n2cc(C3CC3)c3cc(CNC4(C)CCC4)[nH]c3c2=O)c1]

The title compound was prepared in a similar fashion to that described for example 53 using 1-methylcyclobutan-1-amine hydrochloride for reductive amination step. ¹H NMR (400 MHz, CDCl₃) δ 9.97 (s, 1H), 7.68 (d, J=1.4 Hz, 1H), 7.55 (dd, J=8.6, 5.5 Hz, 1H), 7.34 (dd, J=8.9, 2.8 Hz, 1H), 7.27-7.19 (m, 2H), 7.13 (d, J=1.2 Hz, 1H), 6.82 (d, J=1.2 Hz, 1H), 6.45 (d, J=1.3 Hz, 1H), 6.35 (s, 1H), 3.86 (s, 2H), 3.07 (s, 3H), 1.99-1.69 (m, 8H), 1.26 (s, 4H), 0.98-0.88 (m, 2H), 0.92-0.79 (m, 4H), 0.72-0.62 (m, 2H). ESI MS [M+H]⁺ for C₃₄H₃₅FN₆O, calcd 563.3, found 563.3.

### Example 56: 2-({(1S,4R)-2-azabicyclo[2.2.1]hept-2-yl}methyl)-4-cyclopropyl-6-{6-cyclopropyl-4-[4-fluoro-2-(1-methyl-2-imidazolyl)phenyl]-2-pyridyl}-1,6-dihydro-1,6-diaza-7-indenone

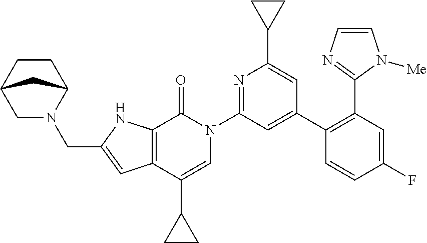 [SMILES: Cn1ccnc1-c1cc(F)ccc1-c1cc(C2CC2)nc(-n2cc(C3CC3)c3cc(CN4C[C@@H]5CC[C@H]4C5)[nH]c3c2=O)c1]

The title compound was prepared in a similar fashion to that described for example 53 using (1S,4R)-2-azabicyclo[2.2.1]heptane hydrochloride for the reductive amination step. ¹H NMR (400 MHz, Methanol-d₄) δ 7.76 (dd, J=8.6, 5.5 Hz, 1H), 7.46 (td, J=8.4, 2.8 Hz, 1H), 7.38 (dd, J=8.9, 2.7 Hz, 1H), 7.28 (d, J=1.4 Hz, 1H), 7.13 (d, J=1.3 Hz, 1H), 7.09 (d, J=1.4 Hz, 1H), 7.07 (d, J=1.2 Hz, 1H), 6.86 (s, 1H), 6.83 (d, J=1.4 Hz, 1H), 4.55 (d, J=14.0 Hz, 1H), 4.42 (d, J=14.0 Hz, 1H), 4.10 (s, 1H), 3.30-3.16 (m, 2H), 3.24 (s, 3H), 2.76 (s, 1H), 2.14-1.72 (m, 7H), 1.65-1.48 (m, 1H), 1.05-0.95 (m, 2H), 0.96-0.81 (m, 4H), 0.73-0.61 (m, 2H). ESI MS [M+H]⁺ for C₃₅H₃₅FN₆O, calcd 575.3, found 575.2.

### Example 57: 2-{[(R)-tetrahydro-2H-pyran-3-ylamino]methyl}-4-cyclopropyl-6-{6-cyclopropyl-4-[4-fluoro-2-(1-methyl-2-imidazolyl)phenyl]-2-pyridyl}-1,6-dihydro-1,6-diaza-7-indenone

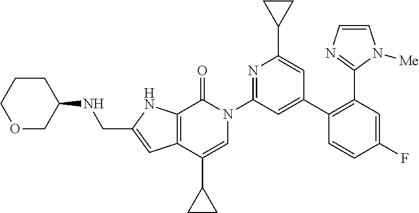 [SMILES: Cn1ccnc1-c1cc(F)ccc1-c1cc(C2CC2)nc(-n2cc(C3CC3)c3cc(CN[C@@H]4CCCOC4)[nH]c3c2=O)c1]

The title compound was prepared in a similar fashion to that described for example 53 using (3R)-oxan-3-amine hydrochloride for the reductive amination step. ¹H NMR (400 MHz, CD₃OD) δ 7.75 (dd, J=8.7, 5.5 Hz, 1H), 7.42 (td, J=8.4, 2.7 Hz, 1H), 7.35 (dd, J=8.9, 2.7 Hz, 1H), 7.32 (d, J=1.3 Hz, 1H), 7.08 (d, J=1.2 Hz, 1H), 7.05 (d, J=1.3 Hz, 1H), 7.02 (d, J=1.1 Hz, 1H), 6.75 (d, J=1.4 Hz, 1H), 6.54 (s, 1H), 3.99-3.93 (m, 2H), 3.90 (ddd, J=11.2, 4.1, 1.9 Hz, 1H), 3.76 (dt, J=11.3, 4.0 Hz, 1H), 3.41 (ddd, J=11.2, 10.0, 2.9 Hz, 1H), 3.22 (dd, J=11.1, 8.6 Hz, 1H), 3.18 (s, 3H), 2.67 (tt, J=8.4, 3.9 Hz, 1H), 2.09-1.83 (m, 3H), 1.79-1.39 (m, 3H), 1.01-0.94 (m, 2H), 0.95-0.84 (m, 6H), 0.72-0.63 (m, 2H). ESI MS [M+H]⁺ for C₃₄H₃₅FN₆O₂, calcd 579.3, found 579.2.

### Example 58: 2-{[(S)-3-hydroxy-1-piperidyl]methyl}-4-cyclopropyl-6-{6-cyclopropyl-4-[4-fluoro-2-(1-methyl-2-imidazolyl)phenyl]-2-pyridyl}-1,6-dihydro-1,6-diaza-7-indenone

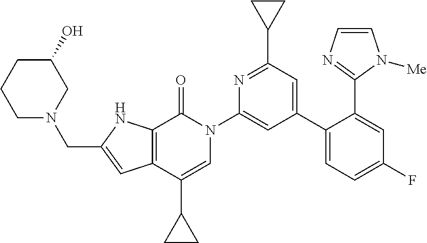 [SMILES: Cn1ccnc1-c1cc(F)ccc1-c1cc(C2CC2)nc(-n2cc(C3CC3)c3cc(CN4CCC[C@H](O)C4)[nH]c3c2=O)c1]

The title compound was prepared in a similar fashion to that described for example 53 using (3S)-piperidin-3-ol hydrochloride for the reductive amination step. ¹H NMR (400 MHz, CDCl₃) δ 11.00 (s, 1H), 7.70 (d, J=1.3 Hz, 1H), 7.56 (dd, J=8.6, 5.5 Hz, 1H), 7.33 (dd, J=8.9, 2.7 Hz, 1H), 7.26-7.19 (m, 2H), 7.11 (d, J=1.2 Hz, 1H), 6.81 (d, J=1.2 Hz, 1H), 6.48 (d, J=1.3 Hz, 1H), 6.34 (d, J=1.9 Hz, 1H), 3.56-3.37 (m, 3H), 3.09 (s, 3H), 2.51-2.38 (m, 1H), 2.33-2.04 (m, 2H), 1.99-1.81 (m, 2H), 1.64-1.51 (m, 2H), 1.38-1.16 (m, 2H), 0.99-0.91 (m, 2H), 0.91-0.80 (m, 6H), 0.70-0.57 (m, 2H). ESI MS [M+H]⁺ for C₃₄H₃₅FN₆O₂, calcd 579.3, found 579.3.

### Example 59: 2-{[(S)-tetrahydro-3-furylamino]methyl}-4-cyclopropyl-6-{6-cyclopropyl-4-[4-fluoro-2-(1-methyl-2-imidazolyl)phenyl]-2-pyridyl}-1,6-dihydro-1,6-diaza-7-indenone

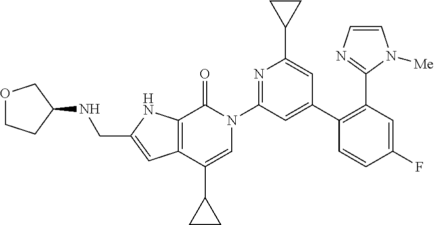 [SMILES: Cn1ccnc1-c1cc(F)ccc1-c1cc(C2CC2)nc(-n2cc(C3CC3)c3cc(CN[C@H]4CCOC4)[nH]c3c2=O)c1]

The title compound was prepared in a similar fashion to that described for example 53 using (3S)-oxolan-3-amine hydrochloride for the reductive amination step. ¹H NMR (400 MHz, CDCl₃) δ 11.19 (s, 1H), 7.62-7.54 (m, 2H), 7.34 (dd, J=8.9, 2.7 Hz, 1H), 7.28-7.22 (m, 1H), 7.17 (d, J=1.1 Hz, 1H), 7.13 (d, J=1.1 Hz, 1H), 6.83 (d, J=1.2 Hz, 1H), 6.53 (d, J=1.3 Hz, 1H), 6.36 (s, 1H), 3.83 (s, 2H), 3.83-3.74 (m, 1H), 3.67 (ddd, J=14.6, 8.6, 5.8 Hz, 2H), 3.45 (dd, J=9.0, 3.8 Hz, 1H), 3.22 (ddd, J=9.7, 7.3, 4.1 Hz, 1H), 3.09 (s, 3H), 2.00-1.83 (m, 2H), 1.63-1.51 (m, 1H), 1.00-0.90 (m, 2H), 0.92-0.80 (m, 6H), 0.74-0.62 (m, 2H). ESI MS [M+H]⁺ for C₃₃H₃₃FN₆O₂, calcd 565.3, found 565.2.

### Example 60: 2-({(1R,4R)-2-oxa-5-azabicyclo[2.2.1]hept-5-yl}methyl)-4-cyclopropyl-6-{6-cyclopropyl-4-[4-fluoro-2-(1-methyl-2-imidazolyl)phenyl]-2-pyridyl}-1,6-dihydro-1,6-diaza-7-indenone

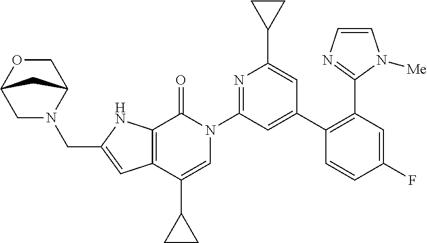 [SMILES: Cn1ccnc1-c1cc(F)ccc1-c1cc(C2CC2)nc(-n2cc(C3CC3)c3cc(CN4C[C@H]5C[C@@H]4CO5)[nH]c3c2=O)c1]

The title compound was prepared in a similar fashion to that described for example 53 using (1R,4R)-2-oxa-5-azabicyclo[2.2.1]heptane hydrochloride for the reductive amination step. ¹H NMR (400 MHz, CDCl₃) δ 9.57 (s, 1H), 7.65 (d, J=1.3 Hz, 1H), 7.54 (dd, J=8.6, 5.5 Hz, 1H), 7.35 (dd, J=8.9, 2.8 Hz, 1H), 7.28-7.20 (m, 2H), 7.14 (d, J=1.2 Hz, 1H), 6.82 (d, J=1.2 Hz, 1H), 6.47 (d, J=1.3 Hz, 1H), 6.37 (d, J=2.1 Hz, 1H), 4.44 (s, 1H), 4.07 (d, J=8.0 Hz, 1H), 3.92 (q, J=14.4 Hz, 2H), 3.67 (dd, J=8.0, 1.8 Hz, 1H), 3.50 (s, 1H), 3.08 (s, 3H), 2.91 (dd, J=10.3, 1.7 Hz, 1H), 2.63 (d, J=10.2 Hz, 1H), 1.98-1.84 (m, 3H), 1.78 (d, J=9.9 Hz, 1H), 0.97-0.76 (m, 6H), 0.75-0.61 (m, 2H). ESI MS [M+H]⁺ for C₃₄H₃₃FN₆O₂, calcd 577.3, found 577.2.

### Example 61: 2-{[(S)-3-methoxy-1-piperidyl]methyl}-4-cyclopropyl-6-{6-cyclopropyl-4-[4-fluoro-2-(1-methyl-2-imidazolyl)phenyl]-2-pyridyl}-1,6-dihydro-1,6-diaza-7-indenone

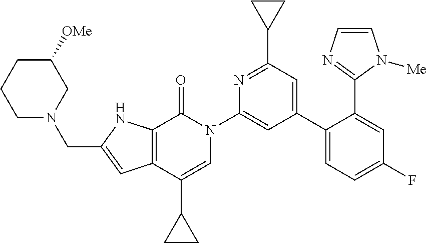 [SMILES: CO[C@H]1CCCN(Cc2cc3c(C4CC4)cn(-c4cc(-c5ccc(F)cc5-c5nccn5C)cc(C5CC5)n4)c(=O)c3[nH]2)C1]

The title compound was prepared in a similar fashion to that described for example 53 using (S)-3-methoxypiperidine for the reductive amination step. ¹H NMR (400 MHz, CDCl₃) δ 9.64 (s, 1H), 7.65 (d, J=1.3 Hz, 1H), 7.54 (dd, J=8.6, 5.6 Hz, 1H), 7.35 (dd, J=8.9, 2.7 Hz, 1H), 7.29-7.22 (m, 1H), 7.23 (d, J=1.2 Hz, 1H), 7.14 (d, J=1.1 Hz, 1H), 6.82 (d, J=1.2 Hz, 1H), 6.47 (d, J=1.3 Hz, 1H), 6.38 (d, J=1.8 Hz, 1H), 3.75-3.63 (m, 2H), 3.34 (s, 3H), 3.33-3.25 (m, 1H), 3.08 (s, 3H), 2.99-2.87 (m, 1H), 2.72-2.58 (m, 1H), 2.12 (dt, J=20.1, 10.0 Hz, 2H), 2.00-1.83 (m, 4H), 1.61-1.45 (m, 1H), 1.35-1.19 (m, 1H), 0.97-0.75 (m, 6H), 0.74-0.61 (m, 2H). ESI MS [M+H]⁺ for C₃₅H₃₇FN₆O₂, calcd 593.3, found 593.2.

### Example 62: 2-[(tert-butylamino)methyl]-4-cyclopropyl-6-{6-cyclopropyl-4-[4-fluoro-2-(1-methyl-2-imidazolyl)phenyl]-2-pyridyl}-1,6-dihydro-1,6-diaza-7-indenone

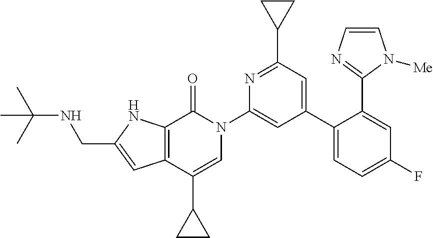 [SMILES: Cn1ccnc1-c1cc(F)ccc1-c1cc(C2CC2)nc(-n2cc(C3CC3)c3cc(CNC(C)(C)C)[nH]c3c2=O)c1]

The title compound was prepared in a similar fashion to that described for example 53 using tert-butylamine for the reductive amination step. ¹H NMR (400 MHz, CDCl₃) δ 8.58 (s, 1H), 7.58 (d, J=1.4 Hz, 1H), 7.55 (dd, J=8.7, 5.5 Hz, 1H), 7.34 (dd, J=8.9, 2.7 Hz, 1H), 7.30-7.20 (m, 1H), 7.14 (d, J=1.2 Hz, 1H), 7.11 (s, 1H), 6.83 (d, J=1.3 Hz, 1H), 6.54 (d, J=1.4 Hz, 1H), 6.22 (s, 1H), 3.96 (s, 2H), 3.08 (s, 3H), 2.04-1.78 (m, 2H), 1.26 (s, 9H), 1.03-0.92 (m, 2H), 0.94-0.81 (m, 4H), 0.73-0.58 (m, 2H). ESI MS [M+H]⁺ for C₃₃H₃₅FN₆O, calcd 551.3, found 551.3.

### Example 63: 6-(2-{[(R)-2-methyl-4-morpholinyl]methyl}-4-cyclopropyl-7-oxo-1,6-dihydro-1,6-diaza-6-indenyl)-4-[4-fluoro-2-(1-methyl-2-imidazolyl)phenyl]-2-pyridinecarbonitrile

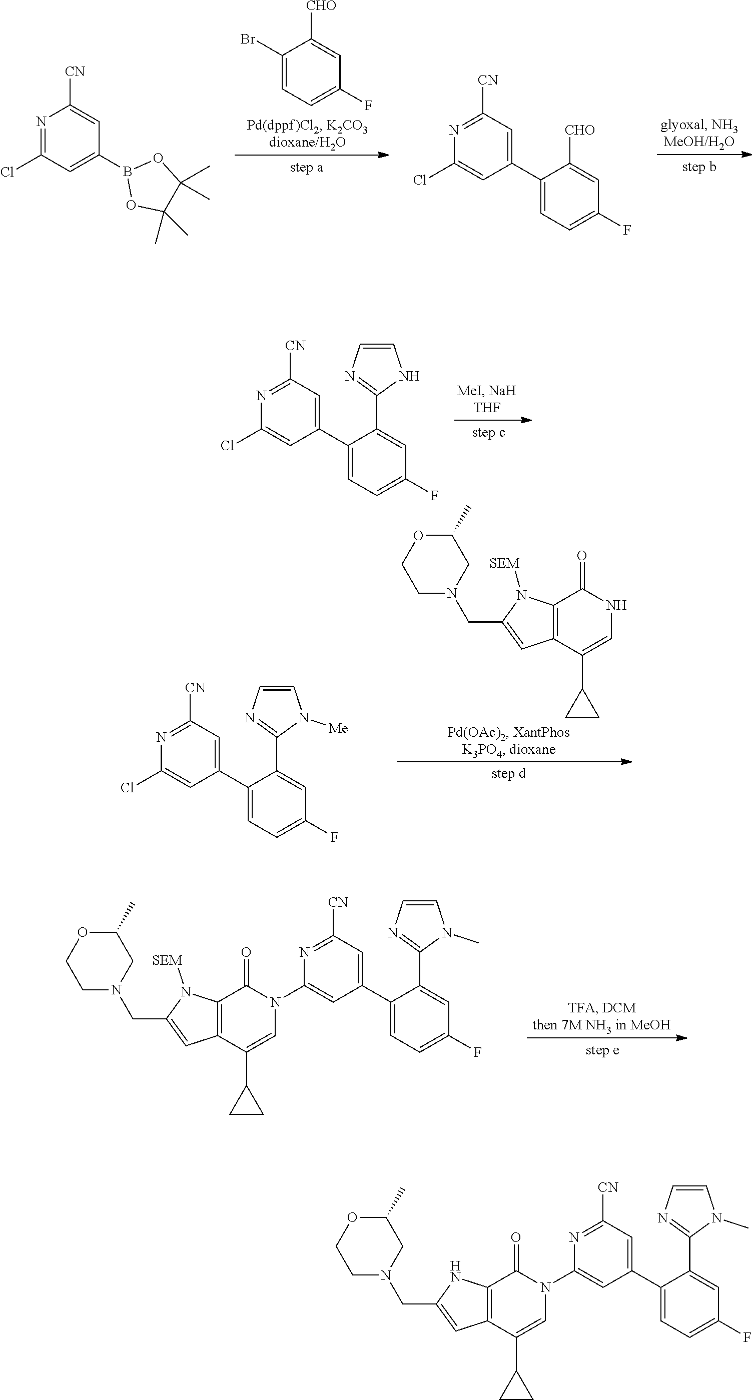

Step a: To a solution of 6-chloro-4-(4,4,5,5-tetramethyl-1,3,2-dioxaborolan-2-yl)pyridine-2-carbonitrile (1.00 g, 3.8 mmol, 1.0 equiv.) and 2-bromo-5-fluorobenzaldehyde (0.77 g, 3.8 mmol, 1.0 equiv.) in dioxane (12 mL) was added Pd(dppf)Cl₂ (0.28 g, 0.38 mmol, 0.1 equiv.) and 1M K₂CO₃ aqueous solution (7.6 mL, 7.6 mmol, 2.0 equiv.). The resulting mixture was degassed by three vacuum/backfilling with nitrogen cycles followed by heating at 100° C. for 2 h. The reaction mixture was cooled to 23° C. and diluted with water (30 mL) and EtOAc (30 mL). The organic phase was separated, and the aqueous layer was extracted with EtOAc (2×20 mL). The combined organic extract was dried over Na₂SO₄ and concentrated under vacuum. The crude residue was purified by column chromatography (SiO₂, 0 to 15% EtOAc gradient in hexanes) to afford 6-chloro-4-(4-fluoro-2-formylphenyl)pyridine-2-carbonitrile.

Step b: To a solution of the product from step a (1.97 g, 3.8 mmol, 1.0 equiv) in methanol (5.0 mL) was added aqueous glyoxal solution (3.1 mL, 27 mmol, 7.0 equiv., 40 wt %) and 7M NH₃ in methanol (5.4 mL, 38 mmol, 10 equiv.) at room temperature. The resulting mixture was stirred at room temperature overnight, and then directly concentrated to dryness under reduced pressure. The crude residue was then purified by column chromatography (SiO₂, 0 to 5% MeOH gradient in dichlorormethane) to afford 6-chloro-4-[4-fluoro-2-(1H-imidazol-2-yl)phenyl]pyridine-2-carbonitrile.

Step c: To a solution of the product from step b (0.190 g, 0.64 mmol, 1.0 equiv.) in THE (5.0 mL) was added NaH (38.4 mg, 0.96 mmol, 1.15 equiv. 60 wt % with mineral oil) at 0° C. The resulting mixture was stirred at this temperature for 10 min before MeI (82 μL, 0.185 g, 1.3 mmol, 2.0 equiv.) was added. The reaction mixture was then stirred at room temperature for 1 h before the quench with sat. aq. NH₄Cl (3 mL) solution. The reaction was diluted with EtOAc (20 mL), the organic phase was separated, and the aqueous layer was extracted with EtOAc (20 mL). The combined organic phase was dried over Na₂SO₄ and concentrated to dryness under reduced pressure. The crude residue was purified by column chromatography (SiO₂, 0 to 50% EtOAc gradient in hexanes) to afford 6-chloro-4-[4-fluoro-2-(1-methylimidazol-2-yl)phenyl]pyridine-2-carbonitrile.

Step d: To a mixture of the product from step c (46.9 mg, 0.15 mmol, 1.0 equiv.) and 4-cyclopropyl-2-[[(2R)-2-methylmorpholin-4-yl]methyl]-1-(2-trimethylsilylethoxymethyl)-6H-pyrrolo[2,3-c]pyridin-7-one (62.6 mg, 0.15 mmol, 1.0 equiv., prepared according to example XX 53 using (2R)-2-methylmorpholine for the reductive amination step) in dioxane (1.5 mL, 0.1 M) were added Pd(OAc)₂ (6.7 mg, 0.030 mmol, 0.20 equiv.), XantPhos (17.4 mg, 0.30 mmol, 0.20 equiv.) and K₃PO₄ (95.5 mg, 0.45 mmol, 3.0 equiv.). The reaction was degassed by sparging with N₂ for 10 min and heated at 100° C. for 1 h under vigorous stirring. The reaction was allowed to cool to room temperature and diluted with water (10 mL) and EtOAc (10 mL). The organic phase was separated, and the aqueous layer was additionally extracted with EtOAc (2×10 mL). The combined organic phase was dried over Na₂SO₄, concentrated under reduced pressure and the crude residue was directly used for the next step without purification.

Step e: To a solution of the crude product from step d (˜0.15 mmol) in dichloromethane (2 mL) was added trifluoracetic acid (1 mL). The resulting solution was stirred at 23° C. for 2 h. The solvent was removed under reduced pressure, and the dry residue was dissolved in 7M NH₃ in MeOH (3 mL). After stirring for 30 min all volatiles were evaporated under vacuum, and the residue was fractionated by reversed phase prep-HPLC (C18 SiO₂, 10-90% CH₃CN in water with 0.1% formic acid) to furnish the title compound. ¹H NMR (400 MHz, CDCl₃) δ 9.58 (s, 1H), 8.27 (d, J=1.4 Hz, 1H), 7.59 (dd, J=8.6, 5.4 Hz, 1H), 7.38-7.27 (m, 3H), 7.13 (d, J=1.2 Hz, 1H), 7.07 (d, J=1.4 Hz, 1H), 6.89 (d, J=1.2 Hz, 1H), 6.42 (d, J=2.1 Hz, 1H), 3.91-3.81 (m, 1H), 3.76-3.56 (m, 4H), 3.26 (s, 3H), 2.79-2.61 (m, 2H), 2.25 (td, J=11.3, 3.3 Hz, 1H), 1.97-1.88 (m, 2H), 1.14 (d, J=6.2 Hz, 3H), 0.97-0.86 (m, 2H), 0.81-0.65 (m, 2H). ESI MS [M+H]⁺ for C₃₂H₃₀FN₇O₂, calcd 564.3, found 564.2.

### Example 64: 2-{[(R)-2-methyl-4-morpholinyl]methyl}-4-cyclopropyl-6-{6-cyclopropyl-4-[2-(1-ethyl-2-imidazolyl)-4-fluorophenyl]-2-pyridyl}-1,6-dihydro-1,6-diaza-7-indenone

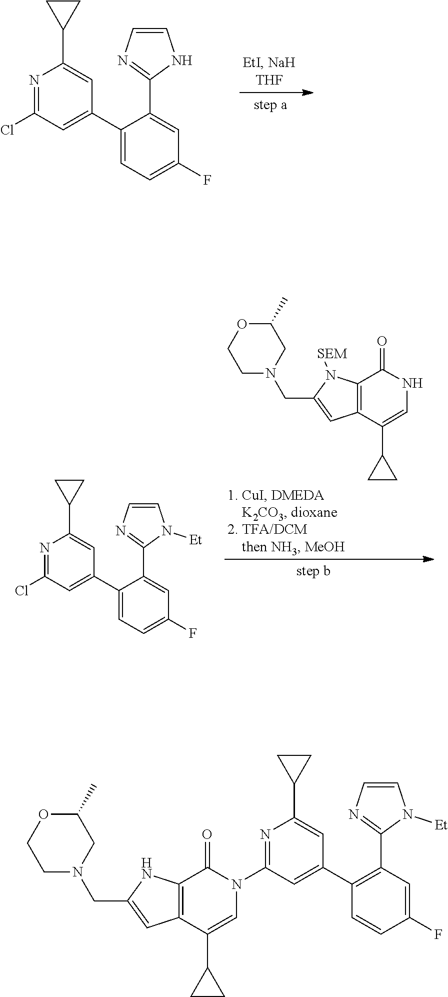 [SMILES: Fc1ccc(-c2cc(Cl)nc(C3CC3)c2)c(-c2ncc[nH]2)c1 ; C[C@@H]1CN(Cc2cc3c(C4CC4)c[nH]c(=O)c3n2COCC[Si](C)(C)C)CCO1 ; CCn1ccnc1-c1cc(F)ccc1-c1cc(Cl)nc(C2CC2)c1 ; CCn1ccnc1-c1cc(F)ccc1-c1cc(C2CC2)nc(-n2cc(C3CC3)c3cc(CN4CCO[C@H](C)C4)[nH]c3c2=O)c1]

Step a: To a solution of 2-chloro-6-cyclopropyl-4-[4-fluoro-2-(1H-imidazol-2-yl)phenyl]pyridine (100 mg, 0.32 mmol, 1.0 equiv., prepared according to example 51) in THE (3.0 mL) was added NaH (14 mg, 0.35 mmol, 1.1 equiv., 60 wt % in mineral oil) at 0° C. The resulting mixture was stirred at this temperature for 10 min before the addition of iodoethane (100 mg, 0.64 mmol, 2.0 equiv.). The reaction mixture was then stirred at room temperature for 1 h before quench with water (2 mL). The product was extracted with EtOAc (3×15 mL), the combined organic extract was dried over Na₂SO₄ and concentrated to dryness under reduced pressure. The crude product was purified by column chromatography (SiO₂, 0 to 40% EtOAc gradient in hexanes) to afford 2-chloro-6-cyclopropyl-4-[2-(1-ethylimidazol-2-yl)-4-fluorophenyl]pyridine.

Step b: To a mixture of the product from step a (51.3 mg, 0.15 mmol, 1.0 equiv.) and 4-cyclopropyl-2-[[(2R)-2-methylmorpholin-4-yl]methyl]-1-(2-trimethylsilylethoxymethyl)-6H-pyrrolo[2,3-c]pyridin-7-one (62.6 mg, 0.15 mmol, 1.0 equiv., prepared according to example 1 using (2R)-2-methylmorpholine for reductive amination step) in dioxane (1.5 mL, 0.1 M) were added CuI (29 mg, 0.15 mmol, 1.0 equiv.), DMEDA (26 mg, 0.30 mmol, 2.0 equiv.) and K₂CO₃ (62 mg, 0.45 mmol, 3.0 equiv.). The reaction mixture was degassed by sparging with nitrogen for 10 min followed by heating at 100° C. overnight under vigorous stirring. The reaction was allowed to cool to 23° C. and diluted with EtOAc (10 mL) and aq. sat. NH₄Cl (10 mL). The organic phase was separated, and the aqueous layer was additionally extracted with EtOAc (10 mL). The combined organic phase was dried over Na₂SO₄ and concentrated to dryness under vacuum. The crude residue was dissolved in dichloromethane (2 mL), followed by the addition of trifluoracetic acid (1 mL). The resulting solution was stirred at 23° C. for 2 h. The solvent was evaporated under vacuum and 7M NH₃ in MeOH (3 mL) was added. After 30 min of stirring at 23° C. all volatiles were removed, and the crude residue was fractionated by reversed phase prep-HPLC (C18 SiO₂, 10-90% CH₃CN in water with 0.1% formic acid) to furnish the title compound. ¹H NMR (400 MHz, DMSO-d₆) δ 12.04 (s, 1H), 7.71 (dd, J=8.7, 5.7 Hz, 1H), 7.53 (td, J=8.5, 2.8 Hz, 1H), 7.43 (dd, J=9.2, 2.8 Hz, 1H), 7.26-7.20 (m, 2H), 6.98 (d, J=1.2 Hz, 1H), 6.96 (d, J=1.1 Hz, 1H), 6.77 (d, J=1.4 Hz, 1H), 6.35 (d, J=2.0 Hz, 1H), 3.78-3.68 (m, 1H), 3.60 (s, 2H), 3.55-3.44 (m, 4H), 2.78-2.61 (m, 2H), 2.07 (td, J=11.1, 3.0 Hz, 1H), 2.05-1.95 (m, 1H), 1.94-1.85 (m, 1H), 1.75 (t, J=10.5 Hz, 1H), 1.03 (d, J=6.2 Hz, 3H), 0.99-0.90 (m, 5H), 0.88-0.82 (m, 2H), 0.83-0.74 (m, 2H), 0.65-0.58 (m, 2H). ESI MS [M+H]⁺ for C₃₅H₃₇FN₆O₂, calcd 593.3, found 593.2.

### Example 65: 2-{[(R)-2-methyl-4-morpholinyl]methyl}-4-cyclopropyl-6-{6-cyclopropyl-4-[4,6-difluoro-2-(1-methyl-2-imidazolyl)phenyl]-2-pyridyl}-1,6-dihydro-1,6-diaza-7-indenone

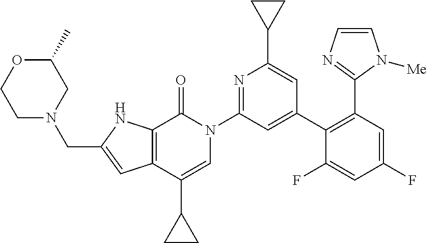 [SMILES: C[C@@H]1CN(Cc2cc3c(C4CC4)cn(-c4cc(-c5c(F)cc(F)cc5-c5nccn5C)cc(C5CC5)n4)c(=O)c3[nH]2)CCO1]

The title compound was prepared in a similar fashion to that described for example 53 starting from 2-chloro-6-cyclopropyl-4-[2,4-difluoro-6-(1-methylimidazol-2-yl)phenyl]pyridine (prepared according to example 51 using 2-bromo-3,5-difluorobenzaldehyde) and 4-cyclopropyl-2-[[(2R)-2-methylmorpholin-4-yl]methyl]-1,6-dihydropyrrolo[2,3-c]pyridin-7-one (prepared according to example 54)¹H NMR (400 MHz, DMSO-d₆) δ 12.05 (s, 1H), 7.65 (ddd, J=10.4, 9.1, 2.6 Hz, 1H), 7.38 (ddd, J=8.9, 2.7, 1.2 Hz, 1H), 7.27 (t, J=1.4 Hz, 1H), 7.12 (d, J=1.2 Hz, 1H), 6.97 (d, J=1.2 Hz, 1H), 6.90 (d, J=1.2 Hz, 1H), 6.87 (d, J=1.2 Hz, 1H), 6.35 (s, 1H), 3.73 (d, J=11.1 Hz, 1H), 3.59 (s, 2H), 3.57-3.43 (m, 2H), 3.26 (s, 3H), 2.77-2.60 (m, 2H), 2.10-2.00 (m, 2H), 1.96-1.85 (m, 1H), 1.75 (t, J=10.6 Hz, 1H), 1.02 (d, J=6.2 Hz, 3H), 1.00-0.93 (m, 2H), 0.88-0.78 (m, 4H), 0.65-0.58 (m, 2H). ESI MS [M+H]⁺ for C₃₄H₃₄F₂N₆O₂, calcd 597.3, found 597.2.

### Example 66: 2-{[(R)-2-methyl-4-morpholinyl]methyl}-4-cyclopropyl-6-{6-cyclopropyl-4-[4,5-difluoro-2-(1-methyl-2-imidazolyl)phenyl]-2-pyridyl}-1,6-dihydro-1,6-diaza-7-indenone

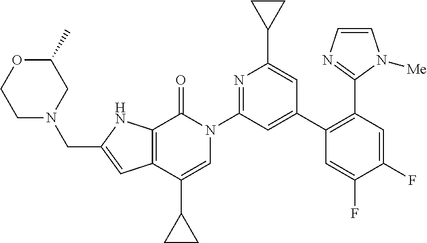 [SMILES: C[C@@H]1CN(Cc2cc3c(C4CC4)cn(-c4cc(-c5cc(F)c(F)cc5-c5nccn5C)cc(C5CC5)n4)c(=O)c3[nH]2)CCO1]

The title compound was prepared in a similar fashion to that described for example 53 starting from 2-chloro-6-cyclopropyl-4-[2,4-difluoro-6-(1-methylimidazol-2-yl)phenyl]pyridine (prepared according to example 51 using 2-bromo-4,5-difluorobenzaldehyde) and 4-cyclopropyl-2-[[(2R)-2-methylmorpholin-4-yl]methyl]-1,6-dihydropyrrolo[2,3-c]pyridin-7-one (prepared according to example 54)¹H NMR (400 MHz, DMSO-d₆) δ 12.05 (s, 1H), 7.80 (dd, J=11.4, 8.0 Hz, 1H), 7.72 (dd, J=10.9, 8.1 Hz, 1H), 7.21 (d, J=1.4 Hz, 1H), 7.15 (d, J=1.2 Hz, 1H), 6.96 (d, J=1.1 Hz, 1H), 6.93 (d, J=1.1 Hz, 1H), 6.81 (d, J=1.4 Hz, 1H), 6.36 (s, 1H), 3.82-3.68 (m, 1H), 3.60 (s, 2H), 3.56-3.43 (m, 2H), 3.19 (s, 3H), 2.80-2.61 (m, 2H), 2.12-1.98 (m, 2H), 1.96-1.84 (m, 1H), 1.76 (t, J=12.5 Hz, 1H), 1.03 (d, J=6.2 Hz, 3H), 1.01-0.92 (m, 2H), 0.90-0.76 (m, 4H), 0.67-0.57 (m, 2H). ESI MS [M+H]⁺ for C₃₄H₃₄F₂N₆O₂, calcd 597.3, found 597.2.

### Example 67: 4-(6-cyclopropyl-2-{4-cyclopropyl-2-[(1-methylcyclobutylamino)methyl]-7-oxo-1,6-dihydro-1,6-diaza-6-indenyl}-4-pyridyl)-3-[1-methyl-4-(trifluoromethyl)-2-imidazolyl]benzonitrile

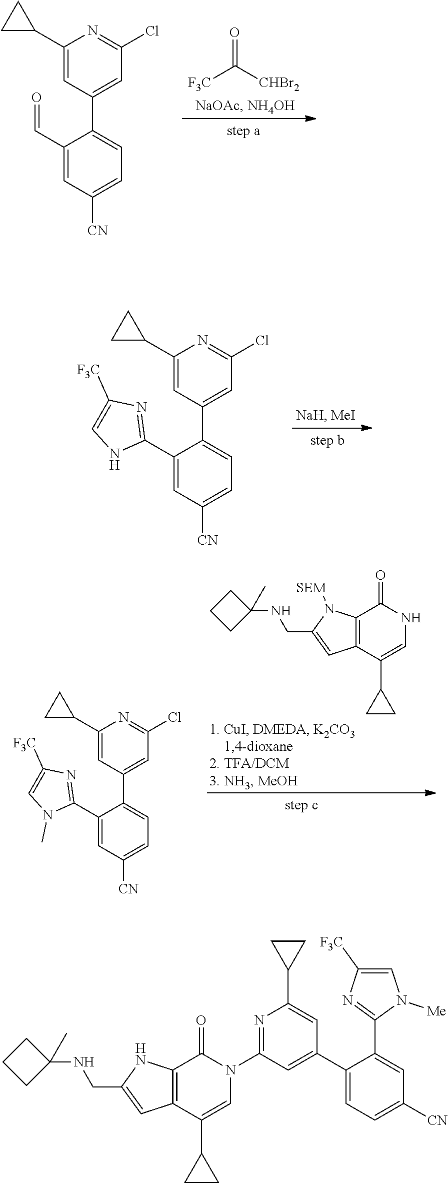

Step a: To a solution of NaOAc (280 mg, 2.8 mmol, 1.1 equiv.) in H₂O (20 mL, 0.25 M) was added 3,3-dibromo-1,1,1-trifluoropropan-2-one (700 mg, 2.6 mmol, 1.0 equiv.). The above solution was heated to 100° C. for 1 h. The mixture was cooled to 0° C., and 4-(2-chloro-6-cyclopropylpyridin-4-yl)-3-(1H-imidazol-2-yl)benzonitrile (800 mg, 2.8 mmol, 1.1 equiv., prepared according to example 13), aqueous concentrated NH₄OH (5 ml, excess) and MeOH (10 ml) were added sequentially. The resulting mixture was stirred at room temperature for 12 h. The resulting mixture was diluted with H₂O (20 ml) and EtOAc (30 mL). The organic phase was separated, and the aqueous layer was additionally extracted with EtOAc (2×15 mL). The combined organic extract was dried over Na₂SO₄ and concentrated to dryness under reduced pressure. The crude product was purified by column chromatography (SiO₂, EtOAc in hexane gradient, 0 to 60%) to afford 4-(2-chloro-6-cyclopropylpyridin-4-yl)-3-[4-(trifluoromethyl)-1H-imidazol-2-yl]benzonitrile.

Step b: To a solution of the product of step a (214 mg, 0.55 mmol, 1.0 equiv.) in THE (5 mL, 0.1 M) was added NaH (45 mg, 1.1 mmol, 2.0 equiv., 60 wt % in mineral oil) at 0° C. MeI (123 mg, 0.83 mmol, 1.5 equiv.) was added after 10 min of stirring. The resulting heterogenous reaction was allowed to warm to room temperature and stirred. After 3 h the resulting mixture was quenched with aqueous saturated NH₄Cl (5 mL) and diluted with EtOAc (20 mL). The organic phase was separated, and the aqueous layer was additionally extracted with EtOAc (2×10 mL). The combined organic phase was dried over Na₂SO₄, concentrated under reduced pressure, and the crude product was purified by column chromatography (SiO₂, MeOH in dichloromethane, 0 to 10% gradient) to furnish 4-(2-chloro-6-cyclopropylpyridin-4-yl)-3-[1-methyl-4-(trifluoromethyl)imidazol-2-yl]benzonitrile.

Step c: To a mixture of the product of step b (50 mg, 0.13 mmol, 1.0 equiv.), 4-cyclopropyl-2-[[(1-methylcyclobutyl)amino]methyl]-1-(2-trimethylsilylethoxymethyl)-6H-pyrrolo[2,3-c]pyridin-7-one (52 mg, 0.13 mmol, 1.0 equiv., prepared according to example 37) in dioxane (3.0 mL) was added CuI (24.5 mg, 0.13 mmol, 1.0 equiv.), N,N′-dimethylethylenediamine (45.4 mg, 0.52 mmol, 4.0 equiv.) and K₂CO₃ (53.4 mg, 0.39 mmol, 3.0 equiv.) After purging with nitrogen for 10 min the reaction mixture was heated at 110° C. overnight. The obtained suspension was cooled to room temperature, then diluted with EtOAc (10 mL) and aqueous saturated NH₄Cl (10 mL). The organic phase was separated, and the aqueous layer was extracted with EtOAc (2×7 mL). The combined extract was sequentially washed with H₂O (10 mL) and brine (10 ml), dried over Na₂SO₄, and concentrated to dryness under reduced pressure. The residual material was dissolved in trifluoroacetic acid/dichloromethane (2 mL, 1:10 v/v), and the resulting solution was stirred at room temperature for 3 h. Upon volatiles removal the material was treated with 7M NH₃ in methanol (2 mL) for 30 min followed by the concentration under vacuum. The crude product was purified by prep-HPLC (C18 SiO₂, 10-90% CH₃CN in water with 0.1% formic acid) to afford title compound. ¹H NMR (400 MHz, CDCl₃) δ 8.01-7.93 (m, 1H), 7.87 (dd, J=8.1, 1.7 Hz, 1H), 7.73 (d, J=1.4 Hz, 1H), 7.70-7.65 (m, 1H), 7.24 (d, J=1.2 Hz, 1H), 7.20 (d, J=1.2 Hz, 1H), 6.57 (d, J=1.4 Hz, 1H), 6.32 (s, 1H), 3.91 (s, 2H), 3.14 (s, 3H), 2.11 (d, J=10.2 Hz, 2H), 1.98-1.83 (m, 4H), 1.79 (td, J=8.6, 4.8 Hz, 2H), 1.35 (s, 3H), 1.06-0.95 (m, 2H), 0.93-0.82 (m, 4H), 0.70-0.60 (m, 2H). ESI MS [M+H]⁺ for C₃₆H₃₅F₃N₇O, calcd 638.3, found 638.1.

### Example 68: 4-(6-cyclopropyl-2-{4-cyclopropyl-2-[(1-methylcyclobutylamino)methyl]-7-oxo-1,6-dihydro-1,6-diaza-6-indenyl}-4-pyridyl)-3-[4-(difluoromethyl)-4H-1,2,4-triazol-3-yl]benzonitrile

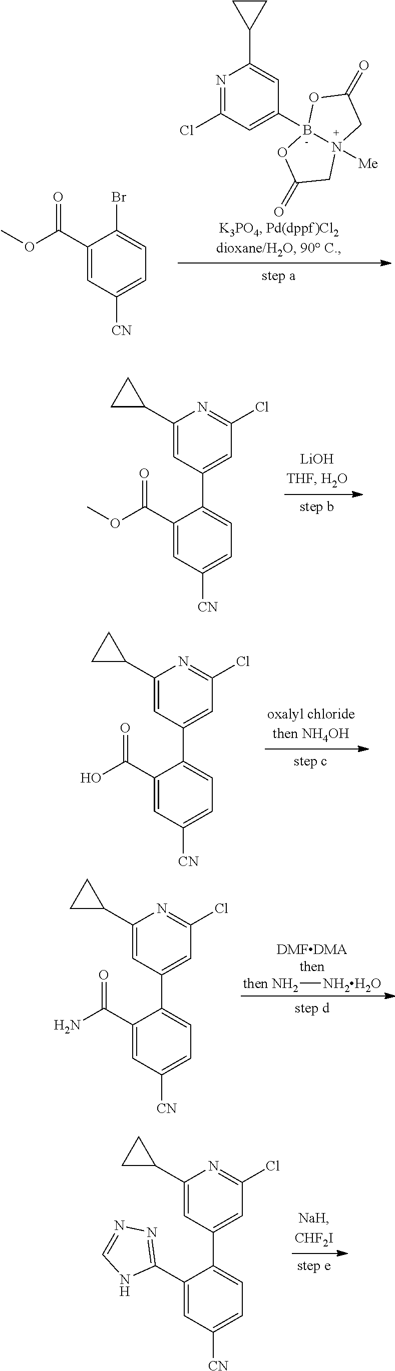

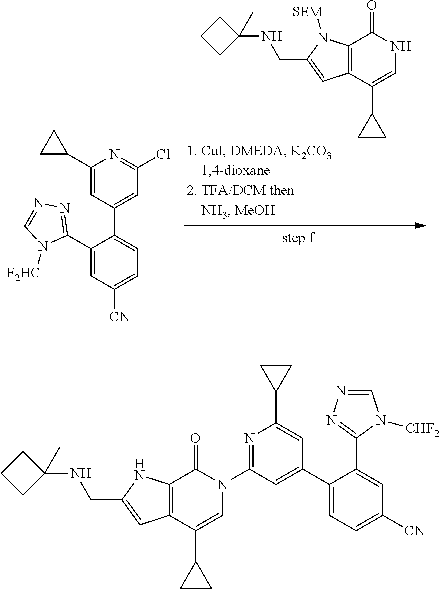 [SMILES: N#Cc1ccc(-c2cc(Cl)nc(C3CC3)c2)c(-c2nncn2C(F)F)c1 ; CC1(NCc2cc3c(C4CC4)c[nH]c(=O)c3n2COCC[Si](C)(C)C)CCC1 ; CC1(NCc2cc3c(C4CC4)cn(-c4cc(-c5ccc(C#N)cc5-c5nncn5C(F)F)cc(C5CC5)n4)c(=O)c3[nH]2)CCC1]

Step a: To a solution of methyl 2-bromo-5-cyanobenzoate (0.78 g, 3.2468 mmol, 1.0 equiv.), 1-(2-chloro-6-cyclopropylpyridin-4-yl)-5-methyl-2,8-dioxa-5-azonia-1-boranuida-bicyclo[3.3.0]octane-3,7-dione (1.0 g, 3.25 mmol, 1.0 equiv., prepared according to protocol described for example 18) and K₃PO₄ (2.1 g, 9.74 mmol, 3.0 equiv.) in dioxane/water mixture (12 mL, 5:1 v/v) was added Pd(dppf)Cl₂ (240 mg, 0.32 mmol, 0.1 equiv.). The reaction mixture was purged with nitrogen for 10 min and heated at 90° C. for 1 h. Once LCMS analysis indicated complete consumption of the ortho-bromoester starting material the mixture was cooled to room temperature and diluted with EtOAc (20 ml) and saturated aqueous NH₄Cl solution. The organic phase was separated, and the aqueous layer was additionally extracted with EtOAc (2×10 mL). The combined organic extract was dried over Na₂SO₄ and concentrated to dryness under reduced pressure. The crude product was purified by column chromatography (SiO₂, hexane in EtOAc, 0 to 60%) to afford methyl 2-(2-chloro-6-cyclopropylpyridin-4-yl)-5-cyanobenzoate.

Step b: To the product of step a (2.52 g, 8.05 mmol, 1.0 equiv.) in THF/H₂O mixture (40 mL, 0.2 M, 3:1 v/v) was added LiOH (600 mg, 24.15 mmol, 3.0 equiv.). The reaction was stirred at room temperature for 3 h. Once complete hydrolysis was observed by LCMS analysis the mixture was acidified to pH 3.0 with aq. 4M HCl at 0° C. The product was extracted with EtOAc (3×20 mL). The combined organic extract was washed with brine (30 mL), dried over Na₂SO₄, and the solvent was evaporated under reduced pressure to yield 2-(2-chloro-6-cyclopropylpyridin-4-yl)-5-cyanobenzoic acid that was used for the next step without purification.

Step c: To the solution of the carboxylic acid product of step b (1.7 g, 5.70 mmol, 1.0 equiv.) in dichloromethane (100 mL, 0.05 M) was added oxalyl chloride (1.05 ml, 11.4094 mmol, 2.0 equiv.) and DMF (5 drops) at 0° C. The mixture was stirred at 0° C. for 3 h followed by volatiles removal under reduced pressure. The crude acid chloride was dissolved in THE /H₂O mixture (90 ml, 2:1 v/v) followed by the dropwise addition of aqueous concentrated NH₄OH (4 ml, excess) dissolved in THF (50 mL) over 10 min. The resulting mixture was stirred at room temperature overnight, then diluted with H₂O (50 mL) and EtOAc (50 ml). The organic phase was separated, and the aqueous layer was extracted with EtOAc (2×30 mL). The combined organic phase was dried over Na₂SO₄ and concentrated to dryness under reduced pressure to furnish 2-(2-chloro-6-cyclopropylpyridin-4-yl)-5-cyanobenzamide.

Step d: A solution of the product of step c (120 mg, 0.36 mmol, 1.0 equiv.) in DMF DMA (18 mL) was heated at 100° C. for 3 h. After cooling to room temperature, the volatiles were removed under vacuum. The crude intermediate was dissolved in acetic acid (15 ml), and hydrazine hydrate (3.5 ml, excess) was added dropwise over 1 min. The resulting mixture was stirred for 2 h at room temperature. Then it was poured in water (40 ml) and EtOAc (40 mL). The organic phase was separated, and the aqueous layer was additionally extracted with EtOAc (2×20 mL). The combined organic extract was sequentially washed with water (3×50 ml) and saturated aqueous NaHCO₃ (50 ml), dried over Na₂SO₄ and concentrated to dryness under reduced pressure. The crude product was purified by column chromatography (SiO₂, MeOH in dichloromethane, 0 to 10%) to yield 4-(2-chloro-6-cyclopropylpyridin-4-yl)-3-(4H-1,2,4-triazol-3-yl)benzonitrile.

Step e: To a solution of the product of step d (380 mg, 1.18 mmol, 1.0 equiv.) in THE (5 mL, 0.2 M) was added NaH (100 mg, 2.36 mmol, 2.0 equiv., 60 wt % in mineral oil) at 0° C. After 10 min difluoroiodomethane (482 mg, 1.77 mmol, 1.5 equiv.) was added in one portion. The resulting mixture was allowed to warm to room temperature and stirred for 3 h. Once TLC analysis indicated complete consumption of the starting material the reaction was quenched with saturated aqueous NH₄Cl solution (3 ml) and diluted with EtOAc (20 mL). The organic phase was separated, and the aqueous layer was extracted with EtOAc (2×10 mL). The combined organic extract was dried over Na₂SO₄ and concentrated to dryness under reduced pressure. The crude product was purified by column chromatography (SiO₂, MeOH in dichloromethane, 0 to 10%) to afford 4-(2-chloro-6-cyclopropylpyridin-4-yl)-3-[4-(difluoromethyl)-1,2,4-triazol-3-yl]benzonitrile.

Step f: To a mixture of the product of step e (37 mg, 0.1 mmol, 1.0 equiv.), 4-cyclopropyl-2-[[(1-methylcyclobutyl)amino]methyl]-1-(2-trimethylsilylethoxymethyl)-6H-pyrrolo[2,3-c]pyridin-7-one (40 mg, 0.1 mmol, 1.0 equiv., prepared according to example 37) in dioxane (3.0 mL) was added CuI (19 mg, 0.1 mmol, 1.0 equiv.), N,N′-dimethylethylenediamine (35.2 mg, 0.4 mmol, 4.0 equiv.) and K₂CO₃ (42 mg, 0.3 mmol, 3.0 equiv.) The resulting mixture was purged with nitrogen for 10 min and heated at 100° C. overnight. The resulting suspension was cooled to room temperature and diluted with EtOAc (10 mL) and aqueous saturated NH₄Cl (10 mL). The organic phase was separated, and the aqueous layer was additionally extracted with EtOAc (2×10 mL). The combined organic extract was washed with water (20 mL), dried over Na₂SO₄ and concentrated to dryness under reduced pressure. The residual material was dissolved in trifluoroacetic acid/dichloromethane (2 mL, 1:10 v/v), and the resulting mixture was stirred at room temperature for 3 h. The solvent was removed under reduced pressure, and the crude intermediate was reconstituted in 7M NH₃ in methanol (2 mL). After 30 min at room temperature the solvent was removed, and the crude product was purified by prep-HPLC (C18 SiO₂, 10-100% CH₃CN in water with 0.1% formic acid) to afford the title compound. ¹H NMR (400 MHz, CDCl₃) δ 8.52 (s, 1H), 8.47 (s, 1H), 7.94 (d, J=1.6 Hz, 1H), 7.90 (dd, J=8.0, 1.7 Hz, 1H), 7.69 (d, J=8.1 Hz, 1H), 7.54 (d, J=1.4 Hz, 1H), 7.18 (d, J=1.2 Hz, 1H), 6.98-6.60 (m, 2H), 6.33 (s, 1H), 3.94 (s, 2H), 2.21 (q, J=9.4, 8.3 Hz, 2H), 1.99 (tt, J=8.0, 4.9 Hz, 1H), 1.92-1.70 (m, 6H), 1.40 (s, 3H), 1.07-0.94 (m, 4H), 0.90-0.81 (m, 2H), 0.72-0.58 (m, 2H). ESI MS [M+H]⁺ for C₃₄H₃₃F₂N₈O, calcd 607.3, found 607.1.

### Example 69: 4-[2-(2-{[(S)-3-methyl-1-piperidyl]methyl}-4-cyclopropyl-7-oxo-1,6-dihydro-1,6-diaza-6-indenyl)-6-cyclopropyl-4-pyridyl]-3-[4-(difluoromethyl)-4H-1,2,4-triazol-3-yl]benzonitrile

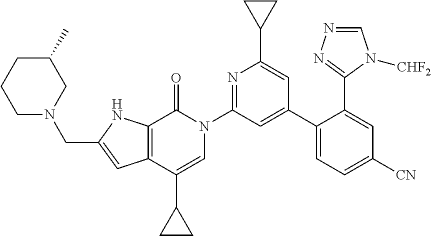 [SMILES: C[C@H]1CCCN(Cc2cc3c(C4CC4)cn(-c4cc(-c5ccc(C#N)cc5-c5nncn5C(F)F)cc(C5CC5)n4)c(=O)c3[nH]2)C1]

The title compound was prepared in a similar fashion to that described for example 68 starting from 4-cyclopropyl-2-[[(3S)-3-methylpiperidin-1-yl]methyl]-1-(2-trimethylsilylethoxymethyl)-6H-pyrrolo[2,3-c]pyridin-7-one (prepared according to example 1). ¹H NMR (400 MHz, CDCl₃) δ 8.54 (s, 1H), 8.07-7.88 (m, 2H), 7.74 (dd, J=8.0, 0.6 Hz, 1H), 7.60 (d, J=1.4 Hz, 1H), 7.33 (d, J=1.3 Hz, 1H), 7.01-6.80 (m, 1H), 6.41 (s, 1H), 3.91 (s, 2H), 3.03 (t, J=31.0 Hz, 2H), 2.24 (s, 2H), 1.99 (tt, J=7.6, 5.1 Hz, 3H), 1.92-1.72 (m, 5H), 1.10-0.93 (m, 4H), 0.92-0.77 (m, 5H), 0.73-0.59 (m, 2H). ESI MS [M+H]⁺ for C₃₅H₃₅F₂N₈O, calcd 621.3, found 621.1.

### Example 70: 2-{[(1S,2R)-2-hydroxycyclopentylamino]methyl}-4-cyclopropyl-6-{2′-[4-(difluoromethyl)-4H-1,2,4-triazol-3-yl]-4′,5-difluoro-3-biphenylyl}-1,6-dihydro-1,6-diaza-7-indenone

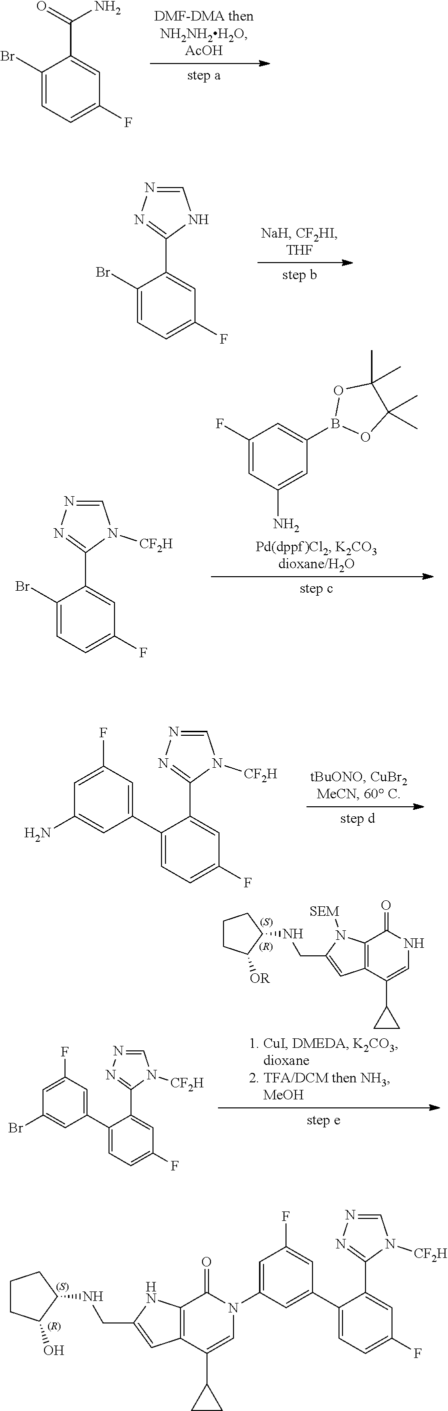

Step a: A solution of the 2-bromo-5-fluorobenzamide (10 g, 45.6 mmol, 1.0 equiv.) in DMF DMA (100 mL) was heated at 100° C. for 3 h. The mixture was allowed to cool to room temperature, and the excess of DMF DMA was removed under vacuum. The crude residue was dissolved in AcOH (75 mL), hydrazine hydrate (18 mL, 365.2 mmol, 8.0 equiv.) was added dropwise over 5 min, and the resulting solution was stirred at room temperature for 12 h. The mixture was slowly basified with aq. sat. NaHCO₃ to pH˜5-6 and the product was extracted with EtOAc (3×75 mL). The combined organic extract was dried over Na₂SO₄ and concentrated to dryness under reduced pressure. The crude product was purified by column chromatography (SiO₂, 0-10% MeOH in dichloromethane) to afford the desired product.

Step b: To a solution of the product of step a (1.5 g, 6.2 mmol, 1.0 equiv.) in THE (30 mL, 0.2 M) was added NaH (0.74 g, 18.6 mmol, 2.0 equiv., 60% wt in oil) at 0° C. in portions over 10 min. To the resulting suspension, CF₂HI was added (6.0 mL, 2M in ACN, 12.4 mmol, 2.0 equiv.) and the mixture was stirred at 23° C. for 3 h. The reaction was quenched with sat. aq. NH₄Cl solution (10 mL) and diluted with EtOAc (30 mL). The organic phase was separated, and the aqueous layer was extracted with EtOAc (20 mL). The combined organic phase was dried over Na₂SO₄, concentrated and the crude residue was purified by column chromatography (SiO₂, 0-80% EtOAc gradient in hexanes) to afford corresponding 1,2,4-triazole derivative.

Step c: The reaction was performed in a similar fashion to Example 17, step b, to afford 3-[2-[4-(difluoromethyl)-1,2,4-triazol-3-yl]-4-fluorophenyl]-5-fluoroaniline as the desired product.

Step d: The reaction was performed in a similar fashion to Example 17, step c to afford 3-[2-(3-bromo-5-fluorophenyl)-5-fluorophenyl]-4-(difluoromethyl)-1,2,4-triazole as the product.

Step e: The reaction was performed in a similar fashion to Example 17, steps d, e, using the product from step d and 4-cyclopropyl-2-[[[(1S,2R)-2-hydroxy-cyclopentyl]amino]methyl]-1,6-dihydropyrrolo[2,3-c]pyridin-7-one (prepared as described in Example 1 using (1R,2S)-2-aminocyclopentan-1-ol for the reductive amination step). ¹H NMR (400 MHz, CDCl₃) δ 12.06 (s, 1H), 8.51 (s, 1H), 7.66 (dd, J=8.6, 5.3 Hz, 1H), 7.45-7.35 (m, 2H), 7.23-7.18 (m, 1H), 7.07-7.00 (m, 1H), 6.96-6.92 (m, 1H), 6.76-6.43 (m, 2H), 6.37 (s, 1H), 3.92-3.71 (m, 3H), 2.83-2.64 (m, 1H), 1.93-1.82 (m, 1H), 1.78-1.49 (m, 5H), 1.45-1.35 (m, 1H), 1.22-1.07 (m, 1H), 0.95-0.80 (m, 3H), 0.72-0.60 (m, 2H); ESI MS [M+H]⁺ C₃₁H₂₈F₄N₆O₂, calcd 593.2, found 593.2.

### Example 71: 4-(6-cyclopropyl-2-{4-cyclopropyl-2-[(1-methylcyclobutylamino)methyl]-7-oxo-1,6-dihydro-1,6-diaza-6-indenyl}-4-pyridyl)-3-(5-methyl-1,3-oxazol-4-yl)benzonitrile

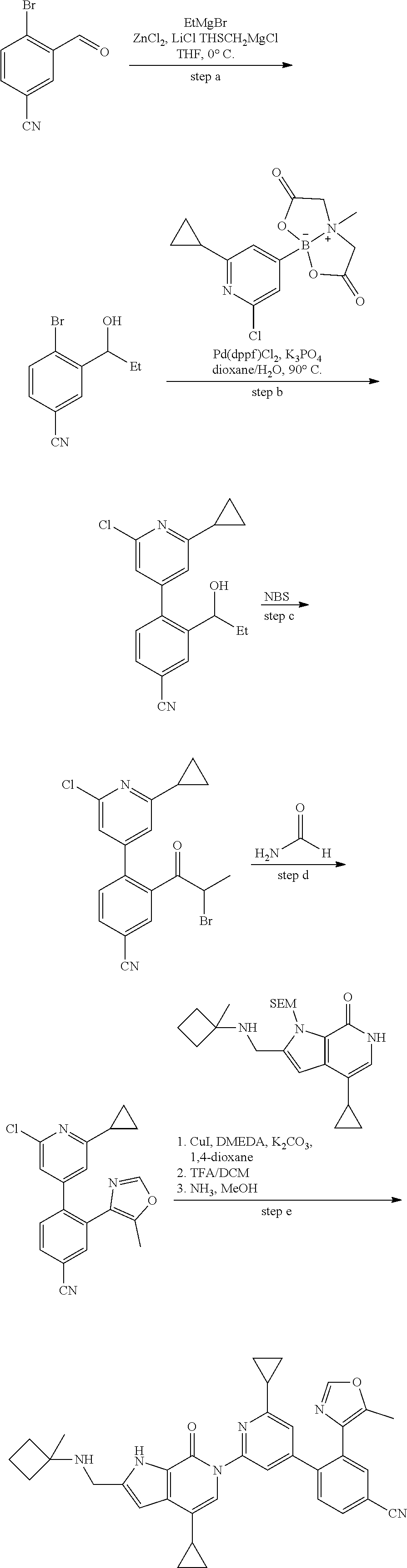

Step a: Anhydrous ZnCl₂ (325 mg, 2.38 mmol, 0.1 equiv.) and LiCl (1.13 g, 26.2 mmol, 1.1 equiv.) were loaded to a round-bottom flask equipped with a stirring bar and nitrogen inlet. (Trimethylsilyl)methylmagnesium chloride (1.0 M in Et₂O, 4.8 ml, 4.76 mmol, 0.2 equiv.) was added to this mixture at room temperature under nitrogen atmosphere. The resulting mixture was stirred for 15 min followed by the addition of EtMgBr (3 M in THF, 8.8 mL, 26.2 mmol, 1 equiv.) and additional 45 min of stirring. The resulting mixture was cooled to 0° C. and 4-bromo-3-formylbenzonitrile (5.0 g, 23.8 mmol, 1.0 equiv.) was added dropwise via syringe pump over 1 h period. The reaction was stirred at 0° C. for 2 h before being quenched with aqueous saturated NH₄Cl (100 mL). The product was extracted with EtOAc (2×70 mL). The combined extract was washed with brine (100 mL), dried over anhydrous MgSO₄ and concentrated to dryness under reduced pressure. The crude product was purified by column chromatography (SiO₂, EtOAc in hexane, 0 to 60%) to afford 4-bromo-3-(1-hydroxypropyl)benzonitrile.

Step b: To a solution of the product from step a (3.1 g, 12.99 mmol, 1.0 equiv.), 1-(2-chloro-6-cyclopropylpyridin-4-yl)-5-methyl-2,8-dioxa-5-azonia-1-boranuidabicyclo[3.3.0]octane-3,7-dione (4.0 g, 12.99 mmol, 1.0 equiv., prepared according to example 18) and K₃PO₄ (8.3 g, 38.97 mmol, 3.0 equiv.) in dioxane/water mixture (72 ml, 6:1 v/v) was added Pd(dppf)Cl₂ (960 mg, 1.3 mmol, 0.1 equiv.) at room temperature. The resulting mixture was degassed by three vacuum/nitrogen backfill cycles and heated at 90° C. for 1 h. Once LCMS analysis indicated the complete consumption of the starting secondary alcohol derivative, the reaction was cooled to room temperature and diluted with water (100 mL) and EtOAc (100 mL). The organic phase was separated, and the aqueous phase was additionally extracted with EtOAc (2×50 mL). The combined organic phase was dried over Na₂SO₄ and concentrated under reduced pressure. The crude product was purified by column chromatography (SiO₂, EtOAc in hexane, 0 to 60% gradient) to afford 4-(2-chloro-6-cyclopropylpyridin-4-yl)-3-(1-hydroxypropyl)benzonitrile.

Step c: To a solution of the product from step b (620 mg, 2.0 mmol, 1.0 equiv.) in dioxane (20 ml) was added NBS (890 mg, 5.0 mmol, 2.5 equiv.), and the mixture was heated at 70° C. for 6 h. The resulting solution was cooled to room temperature and partitioned between EtOAc (50 mL) and water (50 mL). The organic phase was separated, and the aqueous layer was extracted with EtOAc (2×25 mL). The combined organic phase was washed with aq. Na₂S₂O₃ (20 ml) and water (50 ml), dried over Na₂SO₄ and concentrated to dryness under reduced pressure. The crude product was purified by column chromatography (SiO₂, EtOAc in hexanes, 0 to 20%) to give 3-(2-bromopropanoyl)-4-(2-chloro-6-cyclopropylpyridin-4-yl)benzonitrile.

Step d: To a solution of the product from step c (500 mg, 1.3 mmol, 1.0 equiv.) in DMF (20 ml) was added formamide (2 ml, excess). The resulting mixture was heated at 160° C. for 6 h. After cooling to room temperature, the solution was partitioned between water (50 ml) and EtOAc (50 mL). The organic layer was separated, and the aqueous phase was additionally extracted with EtOAc (2×15 mL). The combined extract was sequentially washed with water (2×70 mL) and brine (70 mL), dried over Na₂SO₄, and concentrated to dryness under reduced pressure. The crude material was purified by column chromatography (SiO₂, EtOAc in hexanes, 0 to 30%) to give 4-(2-chloro-6-cyclopropylpyridin-4-yl)-3-(5-methyl-1,3-oxazol-4-yl)benzonitrile.

Step e: To a mixture of the product of step d (70 mg, 0.21 mmol, 1.0 equiv.), 4-cyclopropyl-2-[[(1-methylcyclobutyl)amino]methyl]-1-(2-trimethylsilylethoxymethyl)-6H-pyrrolo[2,3-c]pyridin-7-one (84 mg, 0.21 mmol, 1.0 equiv., prepared according to example 37) in dioxane (3.0 mL) was added CuI (40 mg, 0.21 mmol, 1.0 equiv.), N,N′-dimethylethylenediamine (73.5 mg, 0.84 mmol, 4.0 equiv.) and K₂CO₃ (87 mg, 0.63 mmol, 3.0 equiv.) The resulting mixture was degassed by three sequential cycles of vacuum/N₂ backfill and heated at 110° C. overnight. The obtained suspension was cooled to room temperature and partitioned between EtOAc (10 mL) and aq. sat. NH₄Cl (10 mL). The organic layer was separated, washed with brine (5 mL), dried over Na₂SO₄, and concentrated to dryness under reduced pressure. The crude coupling product was dissolved in trifluoracetic acid/dichloromethane mixture (2 mL, 1:10 v/v), and the solution was stirred at room temperature for 3 h before all volatiles were removed under vacuum. The crude material was redissolved in 7M NH₃ in methanol (2 mL), and the solution was stirred for additional 30 min before final concentration under vacuum. The crude product was then purified by prep-HPLC (C18 SiO₂, 10-100% CH₃CN in water with 0.1% formic acid) to afford the title compound. ¹H NMR (400 MHz, CDCl₃) δ 7.85 (d, J=1.7 Hz, 1H), 7.80 (s, 1H), 7.73 (dd, J=8.0, 1.8 Hz, 1H), 7.65-7.55 (m, 2H), 7.18 (d, J=1.1 Hz, 1H), 6.89 (d, J=1.4 Hz, 1H), 6.21 (s, 1H), 3.85 (s, 2H), 2.18 (s, 4H), 2.08-1.98 (m, 1H), 1.97 (s, 3H), 1.93-1.81 (m, 2H), 1.80-1.70 (m, 2H), 1.35 (s, 3H), 1.07-0.96 (m, 4H), 0.92-0.83 (m, 2H), 0.70-0.59 (m, 2H). ESI MS [M+H]⁺ for C₃₅H₃₅N₆O₂, calcd 571.3, found 571.1.

### Example 72: 4-(6-cyclopropyl-2-{2-[(2-methoxyethylamino)methyl]-7-oxo-1,6-dihydro-1,4,6-triaza-6-indenyl}-4-pyridyl)-3-(5-methyl-1,3-oxazol-4-yl)benzonitrile

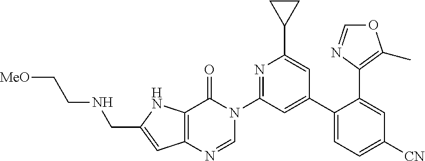 [SMILES: COCCNCc1cc2ncn(-c3cc(-c4ccc(C#N)cc4-c4ncoc4C)cc(C4CC4)n3)c(=O)c2[nH]1]

The title compound was prepared in a similar fashion to that described for example 71 starting from 6-[(2-methoxyethylamino)methyl]-5-(2-trimethylsilylethoxymethyl)-3H-pyrrolo[3,2-d]pyrimidin-4-one (prepared as described in example 22, using methoxyethanamine for reductive amination step). ¹H NMR (400 MHz, CDCl₃) δ 8.30 (s, 1H), 7.86 (d, J=1.7 Hz, 1H), 7.81 (s, 1H), 7.76 (dd, J=8.0, 1.7 Hz, 1H), 7.60 (d, J=8.0 Hz, 1H), 7.41 (d, J=1.3 Hz, 1H), 7.06 (d, J=1.4 Hz, 1H), 6.36 (s, 1H), 4.02 (s, 2H), 3.52 (t, J=4.9 Hz, 2H), 3.38 (s, 3H), 2.85 (t, J=5.0 Hz, 2H), 2.26-1.88 (m, 5H), 1.07-0.96 (m, 4H). ESI MS [M+H]⁺ for C₂₉H₂₇N₇O₃, calcd 522.2, found 522.2.

### Example 73: 4-(6-cyclopropyl-2-{4-cyclopropyl-2-[(1-methylcyclobutylamino)methyl]-7-oxo-1,6-dihydro-1,6-diaza-6-indenyl}-4-pyridyl)-3-[3-(trifluoromethyl)-2-pyridyl]benzonitrile

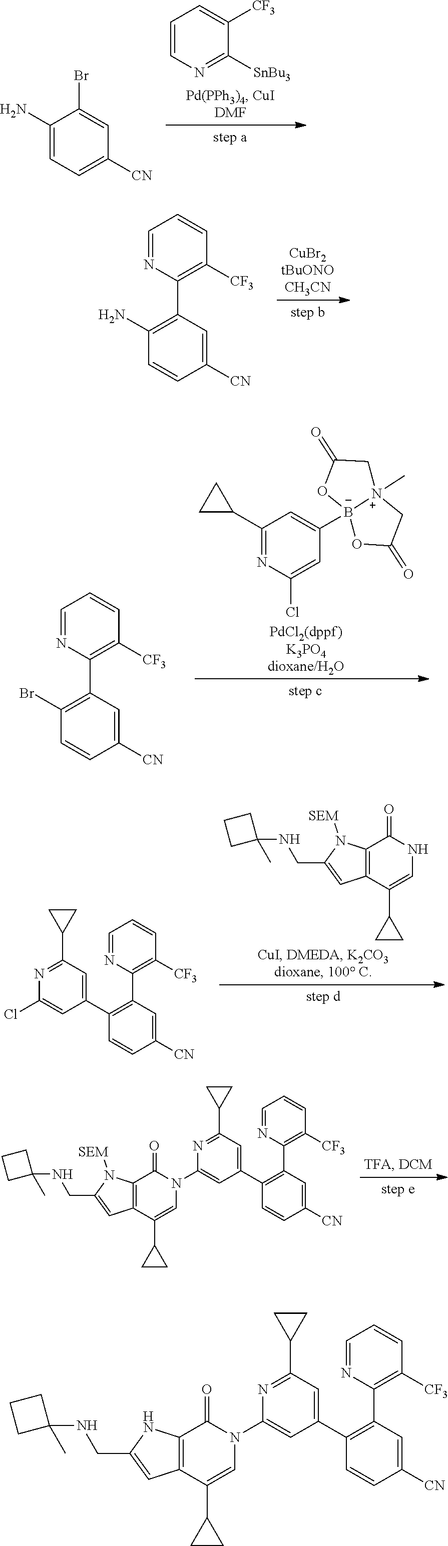

Step a: A mixture of 4-amino-3-bromobenzonitrile (0.23 g, 1.14 mmol, 1 equiv.) and tributyl-[3-(trifluoromethyl)pyridin-2-yl]stannane (0.5 g, 1.14 mmol, 1 equiv.) in DMF (5.7 mL, 0.2M) was degassed by three cycles of vacuum/backfilling with nitrogen followed by the addition of CuI (11 mg, 0.06 mmol, 0.05 equiv.) and Pd(PPh₃)₄ (67 mg, 0.06 mmol, 0.05 equiv.). The resulting mixture was heated at 90° C. for 24 h. The resulting mixture was cooled to 23° C. and diluted with water (30 mL) and EtOAc (30 mL). The organic phase was separated and the aqueous phase was additionally extracted with EtOAc (2×15 mL). The combined organic phase was washed with water (2×30 mL), dried over Na₂SO₄ and concentrated to dryness under reduced pressure. The crude residue was purified by column chromatography (SiO2, 0-100% EtOAc gradient in dichloromethane) to afford 4-amino-3-[3-(trifluoromethyl)pyridin-2-yl]benzonitrile.

Step b: t-BuONO (0.35 mL, 2.9 mmol, 4.5 equiv.) was added to a solution of the product from step a (0.17 g, 0.65 mmol, 1 equiv.) and CuBr₂ (0.29 g, 1.3 mmol, 2 equiv.) in CH₃CN (3.3 mL, 0.2 M) preheated at 60° C. The resulting dark green mixture was stirred at this temperature for 3 h before cooling to room temperature. The resulting solution was diluted with aq. sat. NH₄Cl (10 mL) and EtOAc (20 mL). The organic phase was separated, and the aqueous phase was additionally extracted with EtOAc (10 mL). The combined organic phase was dried over Na₂SO₄ and concentrated to dryness under reduced pressure. The crude residue was purified by column chromatography (SiO₂, 0-100% EtOAc gradient in hexanes) to afford 4-bromo-3-[3-(trifluoromethyl)pyridin-2-yl]benzonitrile.

Step c: A mixture of the bromide product from step b (0.17 g, 0.52 mmol, 1 equiv.) and 1-(2-chloro-6-cyclopropylpyridin-4-yl)-5-methyl-2,8-dioxa-5-azonia-1-boranuida-bicyclo[3.3.0]octane-3,7-dione (0.21 g, 0.68 mmol, 1.3 equiv., prepared according to example 18) and K₃PO₄ (0.33 g, 1.56 mmol, 3 equiv.) in dioxane/water mixture (5 mL, 4:1 v/v) was degassed by three cycles of vacuum/backfilling with nitrogen followed by the addition of PdCl₂(dppf) (38 mg, 0.05 mmol, 0.1 equiv.). The resulting mixture was heated at 80° C. for 2 h before cooling to room temperature and dilution with water (10 mL) and EtOAc (15 mL). The organic phase was separated, and the aqueous phase was additionally extracted with EtOAc (10 mL). The combined organic phase was dried over Na₂SO₄ and concentrated to dryness under reduced pressure. The crude residue was purified by column chromatography (SiO₂, 0-100% EtOAc gradient in hexanes) to afford 4-(2-chloro-6-cyclopropylpyridin-4-yl)-3-[3-(trifluoromethyl)pyridin-2-yl]benzonitrile.

Step d: To a mixture of the product from step c (60 mg, 0.15 mmol, 1.0 equiv.) and 4-cyclopropyl-2-[[(1-methylcyclobutyl)amino]methyl]-1-(2-trimethylsilylethoxymethyl)-6H-pyrrolo[2,3-c]pyridin-7-one (50 mg, 0.15 mmol, 1.0 equiv., prepared according example 37) in dioxane (1.5 mL, 0.05 M) was added CuI (29 mg, 0.15 mmol, 1.0 equiv.), DMEDA (27 mg, 0.30 mmol, 2.0 equiv.) and K₂CO₃ (62 mg, 0.45 mmol, 3.0 equiv.). The resulting mixture was sparged with nitrogen for 10 min and heated at 100° C. under N₂ for 16 h under vigorous stirring. The reaction was allowed to cool to 23° C., diluted with aq. sat. NH₄Cl (3 mL) and EtOAc (10 mL). The organic phase was separated, and the aqueous layer was additionally extracted with EtOAc (10 mL). The combined organic phase was dried over MgSO₄ and concentrated to dryness under vacuum. The crude residue was purified by column chromatography (0 to 20% MeOH gradient in dichloromethane) to provide corresponding coupling product.

Step e: The product from step d was treated with a mixture of trifluoracetic acid/dichloromethane (4.0 mL, 1:1 v/v) at room temperature for 1 h. Upon concentration to dryness the residue was fractionated by reversed phase prep-HPLC (C18 column, 10 to 100% MeCN gradient in water with 0.1% formic acid) to furnish the title compound. ¹H NMR (400 MHz, CDCl₃) δ 8.77 (d, J=4.9 Hz, 1H), 8.02 (d, J=8.3 Hz, 1H), 7.83 (d, J=8.0 Hz, 1H), 7.72-7.62 (m, 2H), 7.55-7.43 (m, 2H), 6.99 (s, 1H), 6.77 (s, 1H), 6.15 (s, 1H), 3.92 (s, 1H), 2.23 (s, 2H), 2.10 (s, 1H), 2.02 (s, 1H), 1.88 (s, 6H), 1.45 (s, 2H), 0.97 (s, 2H), 0.86 (d, J=15.5 Hz, 7H), 0.59 (s, 1H). ESI MS [M+H]⁺ for C₃₇H₃₄F₃N₅O, calcd 635.3, found 635.2.

### Example 74: 4-[2-cyclopropyl-6-[4-cyclopropyl-2-[[(3S)-3-methylpiperidin-1-yl]methyl]-7-oxo-1H-pyrrolo[2,3-c]pyridin-6-yl]pyridin-4-yl]-3-(3-methylpyridin-2-yl)benzonitrile

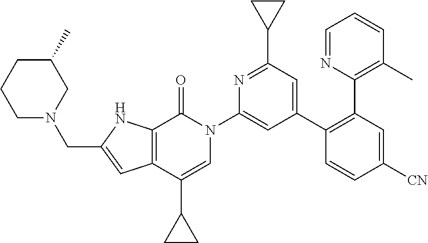 [SMILES: Cc1cccnc1-c1cc(C#N)ccc1-c1cc(C2CC2)nc(-n2cc(C3CC3)c3cc(CN4CCC[C@H](C)C4)[nH]c3c2=O)c1]

The title compound was prepared in a similar fashion to that described for example 73 starting from tributyl-(3-methylpyridin-2-yl)stannane in step a and using 4-cyclopropyl-2-[[(3S)-3-methylpiperidin-1-yl]methyl]-1,6-dihydropyrrolo[2,3-c]pyridin-7-one (prepared according to example 1) in step d. ¹H NMR (400 MHz, CDCl₃) δ 9.82 (br. s, 1H), 8.54 (dd, J=4.8, 1.6 Hz, 1H), 7.78 (dd, J=8.0, 1.7 Hz, 1H), 7.75 (d, J=1.6 Hz, 1H), 7.69 (d, J=1.4 Hz, 1H), 7.65 (d, J=8.0 Hz, 1H), 7.44 (dd, J=7.9, 1.6 Hz, 1H), 7.25-7.17 (m, 2H), 6.49 (s, 1H), 6.36 (s, 1H), 3.71-3.49 (m, 2H), 2.88-2.64 (m, 2H), 2.03-1.79 (m, 6H), 1.76-1.46 (m, 5H), 0.95-0.77 (m, 7H), 0.74-0.59 (m, 4H). ESI MS [M+H]⁺ for C₃₈H₃₈N₆O, calcd 595.3, found 595.3.

### Example 75: 4-[2-Cyclopropyl-6-[4-cyclopropyl-2-[[(1-methylcyclobutyl)amino]methyl]-7-oxo-1H-pyrrolo[2,3-c]pyridin-6-yl]pyridin-4-yl]-3-(4-methylpyridazin-3-yl)benzonitrile

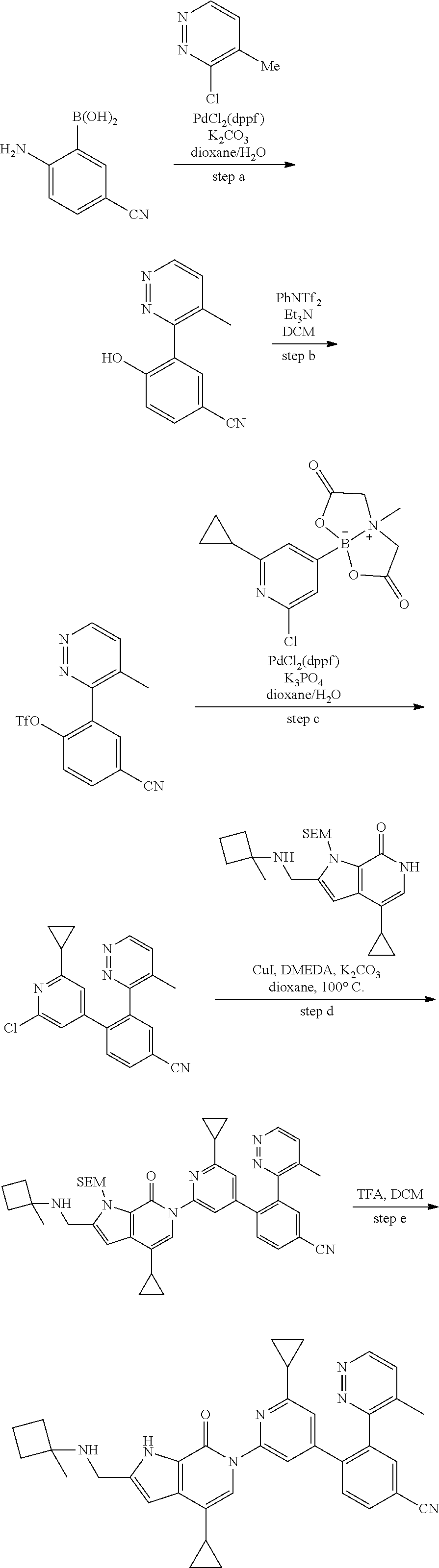

Step a: A mixture of (5-cyano-2-hydroxyphenyl)boronic acid (0.63 g, 3.9 mmol, 1 equiv.), 3-chloro-4-methylpyridazine (0.5 g, 3.9 mmol, 1 equiv.) and K₂CO₃ (1.6 g, 11.7 mmol, 3 equiv.) in dioxane/water mixture (13 mL, 4:1 v/v) was degassed by three cycles of vacuum/backfilling with nitrogen followed by the addition of PdCl₂(dppf) (0.29 g, 0.4 mmol, 0.1 equiv.). The resulting mixture was heated at 90° C. overnight, then cooled to room temperature and concentrated to dryness under reduced pressure. The crude residue was fractionated by reversed phase column chromatography (C18 SiO₂, 0 to 100% CH₃CN in water with 0.1% formic acid) to afford 4-hydroxy-3-(4-methylpyridazin-3-yl)benzonitrile.

Step b: Triethylamine (0.4 mL, 2.7 mmol, 3 equiv.) and PhNTf₂ (0.42 g, 1.17 mmol, 3 equiv.) were added sequentially to a suspension of the product from step a (0.19 g, 0.9 mmol, 1 equiv.) in dichloromethane (4.5 ml, 0.2 M) at room temperature. The resulting mixture was stirred for 4 h at room temperature. Once TLC analysis indicated complete conversion of the starting material the mixture was concentrated to dryness, and the dry residue was directly purified by column chromatography (SiO₂, 0-100% EtOAc gradient in hexanes) to afford [4-cyano-2-(4-methylpyridazin-3-yl)phenyl] trifluoromethanesulfonate.

Step c: A mixture of the triflate product from step b (0.27 g, 0.79 mmol, 1 equiv.) and 1-(2-chloro-6-cyclopropylpyridin-4-yl)-5-methyl-2,8-dioxa-5-azonia-1-boranuidabicyclo[3.3.0]octane-3,7-dione (0.24 g, 0.79 mmol, 1 equiv., prepared according to example 18) and K₃PO₄ (0.5 g, 2.4 mmol, 3 equiv.) in dioxane/water mixture (8 mL, 4:1 v/v) was degassed by three cycles of vacuum/backfilling with nitrogen followed by the addition of PdCl₂(dppf) (58 mg, 0.08 mmol, 0.1 equiv.). The resulting mixture was heated at 80° C. for 1 h before cooling to room temperature and dilution with water (10 mL) and EtOAc (20 mL). The organic phase was separated, and the aqueous phase was additionally extracted with EtOAc (20 mL). The combined organic phase was dried over Na₂SO₄ and concentrated to dryness under reduced pressure. The crude residue was purified by column chromatography (SiO2, 0-100% EtOAc gradient in hexanes) to afford 4-(2-chloro-6-cyclopropylpyridin-4-yl)-3-(4-methylpyridazin-3-yl)benzonitrile.

Steps d and e: Steps d and e were performed in a similar fashion to example 73 to afford the title compound. ¹H NMR (400 MHz, CDCl₃) δ 9.69 (br. s, 1H), 9.06 (d, J=5.3 Hz, 1H), 7.87 (dd, J=8.0, 1.7 Hz, 1H), 7.82 (d, J=1.6 Hz, 1H), 7.67 (d, J=8.0 Hz, 1H), 7.64 (s, 1H), 7.26-7.19 (m, 2H), 6.58 (s, 1H), 6.35 (s, 1H), 3.88 (s, 2H), 2.02-1.91 (m, 4H), 1.89-1.57 (m, 8H), 1.29 (s, 3H), 1.02-0.74 (m, 8H), 0.67-0.62 (m, 2H). ESI MS [M+H]⁺ for C₃₆H₃₅N₇O, calcd 582.3, found 582.3.

### Example 76: 4-[2-Cyclopropyl-6-[4-cyclopropyl-2-[[[1-(hydroxymethyl)cyclobutyl]-amino]methyl]-7-oxo-1H-pyrrolo[2,3-c]pyridin-6-yl]pyridin-4-yl]-3-(4-methylpyridazin-3-yl)benzonitrile

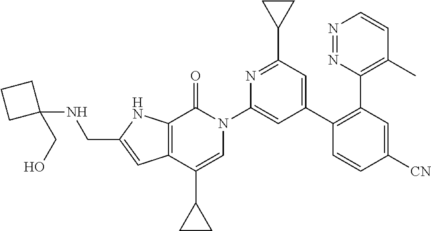 [SMILES: Cc1ccnnc1-c1cc(C#N)ccc1-c1cc(C2CC2)nc(-n2cc(C3CC3)c3cc(CNC4(CO)CCC4)[nH]c3c2=O)c1]

The title compound was prepared in a similar fashion to that described for example 75 using 4-cyclopropyl-2-[[[1-(hydroxymethyl)cyclobutyl]amino]methyl]-1,6-dihydro-pyrrolo[2,3-c]pyridin-7-one (prepared according to example 1 using (1-aminocyclo-butyl)methanol on step e). ¹H NMR (400 MHz, CDCl₃) δ 10.95 (s, 1H), 9.00 (d, J=4.9 Hz, 1H), 7.85 (d, J=8.0 Hz, 1H), 7.80 (d, J=1.6 Hz, 1H), 7.66 (d, J=8.0 Hz, 1H), 7.53 (s, 1H), 7.24 (d, J=5.5 Hz, 1H), 7.19-7.10 (m, 1H), 6.66 (s, 1H), 6.36 (s, 1H), 3.82 (s, 2H), 3.58 (s, 2H), 2.03-1.80 (m, 9H), 1.73 (d, J=12.3 Hz, 2H), 1.00-0.74 (m, 6H), 0.70-0.53 (m, 2H). ESI MS [M+H]⁺ for C₃₆H₃₅N₇O₂, calcd 598.3, found 598.3.

### Example 77: 4-[2-Cyclopropyl-6-[4-cyclopropyl-2-[[[1-(hydroxymethyl)cyclobutyl]amino]methyl]-7-oxo-1H-pyrrolo[2,3-c]pyridin-6-yl]pyridin-4-yl]-3-(4-methylpyridazin-3-yl)benzonitrile

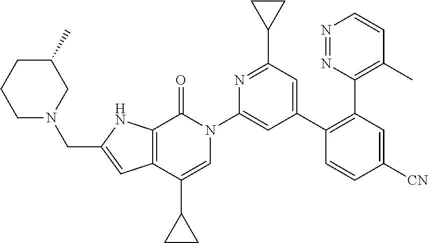 [SMILES: Cc1ccnnc1-c1cc(C#N)ccc1-c1cc(C2CC2)nc(-n2cc(C3CC3)c3cc(CN4CCC[C@H](C)C4)[nH]c3c2=O)c1]

The title compound was prepared in a similar fashion to that described for example 75 using 4-cyclopropyl-2-[[(3S)-3-methylpiperidin-1-yl]methyl]-1,6-dihydropyrrolo[2,3-c]pyridin-7-one (prepared according to example 1). ¹H NMR (400 MHz, CDCl₃) δ 9.65 (br. s, 1H), 9.06 (d, J=5.2 Hz, 1H), 7.86 (dd, J=8.0, 1.7 Hz, 1H), 7.83 (d, J=1.6 Hz, 1H), 7.73-7.60 (m, 2H), 7.25-7.20 (m, 2H), 6.57 (s, 1H), 6.35 (s, 1H), 3.88-3.30 (m, 2H), 2.76 (dd, J=19.9, 9.2 Hz, 2H), 1.99-1.78 (m, 6H), 1.76-1.45 (m, 5H), 0.99-0.73 (m, 8H), 0.69-0.57 (m, 2H). ESI MS [M+H]⁺ for C₃₇H₃₇N₇O, calcd 596.3, found 596.3.

### Example 78: 6-{6-cyclopropyl-4-[4-fluoro-2-(5-methyl-1,3-oxazol-4-yl)phenyl]-2-pyridyl}-2-[(2-methoxyethylamino)methyl]-1,6-dihydro-1,4,6-triaza-7-indenone

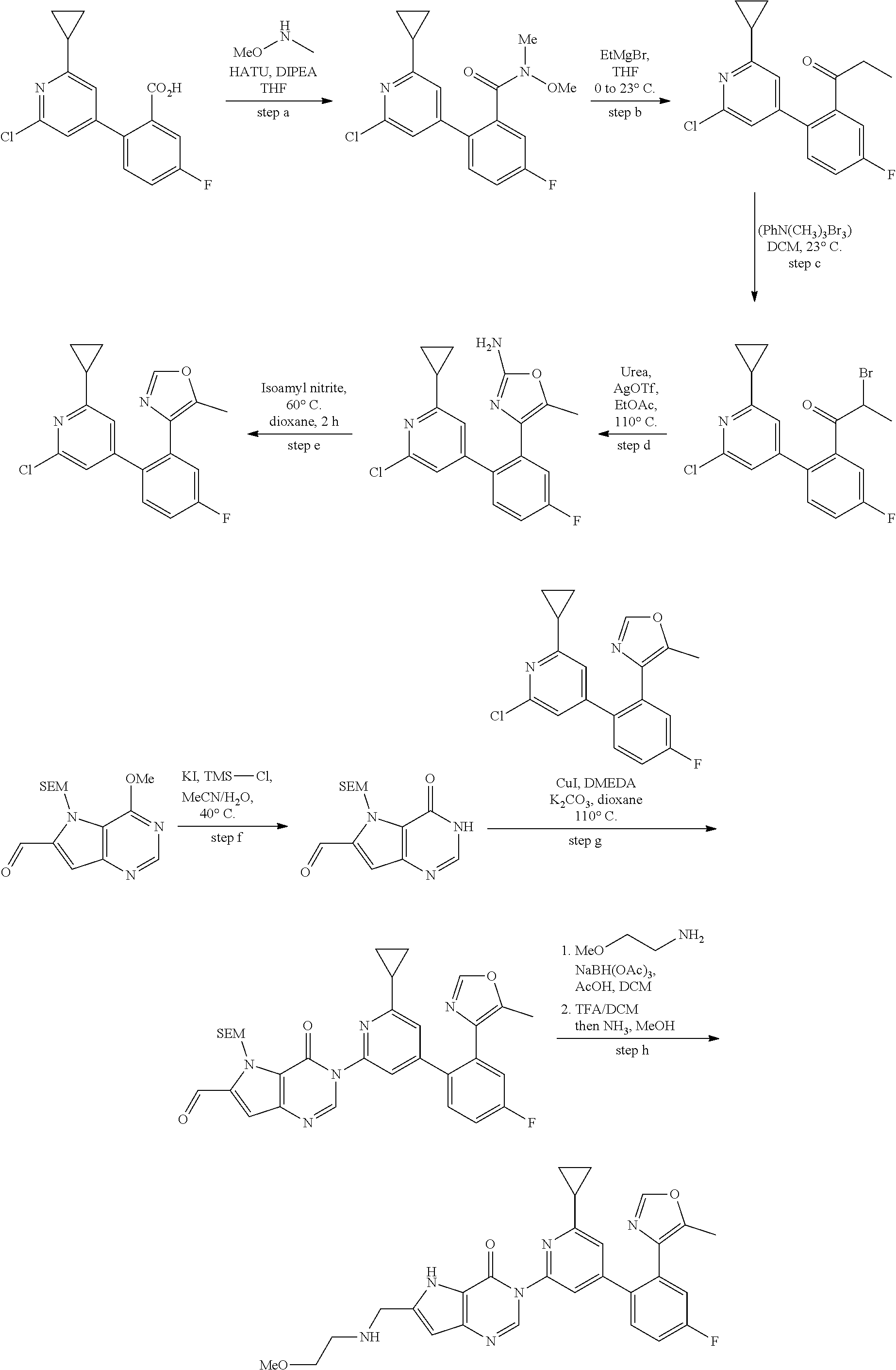

Step a: To a solution of the 2-(2-chloro-6-cyclopropylpyridin-4-yl)-5-fluorobenzoic acid (8.0 g, 27.4 mmol, 1.0 equiv.), N-methoxymethanamine (4.0 g, 41.2 mmol, 1.5 equiv.) and HATU (13.5 g, 35.7 mmol, 1.5 equiv.) in THE (100 mL) was added DIPEA (14.3 mL, 82.4 mmol, 3.0 equiv.). The resulting mixture was stirred at room temperature for 12 h and then concentrated under vacuum to dryness. The dry residue was partitioned between EtOAc (50 mL) and water (25 mL). The organic phase was separated, and the aqueous layer was additionally extracted with EtOAc (2×50 mL). The combined organic extract was dried over Na₂SO₄ and concentrated. The crude product was purified by column chromatography (SiO₂, 0-50% EtOAc gradient in hexanes) to afford the desired product.

Step b: To a solution of the product from step a (8.3 g, 25.1 mmol, 1.0 equiv.) in THE (83 ml, 0.3 M) was added EtMgBr (3 M in THE 12.5 mL, 37.6 mmol, 1.5 equiv) at 0° C. The resulting mixture was stirred at room temperature for 8 h, then quenched with aq. sat. NH₄Cl (20 mL) and diluted with EtOAc (100 mL) and water (30 mL). The organic phase was separated, and the aqueous phase was additionally extracted with EtOAc (2×40 mL). The combined organic extract was washed with brine (20 mL), dried over Na₂SO₄ and concentrated to dryness under reduced pressure. The crude product was purified by column chromatography (SiO₂, 0-50% EtOAc gradient in hexanes) to afford the desired product.

Step c: To a solution of the product from step b (5.7 g, 18.7 mmol, 1.0 equiv.) in dichloromethane (60 ml) was added trimethylphenylammonium tribromide (7.39 g, 19.6 mmol, 1.05 equiv.). The resulting mixture was stirred at room temperature for 48 h. After completion, the solution was concentrated to dryness under reduced pressure, and partitioned between EtOAc (100 mL) and water (50 mL). The organic phase was separated, and the aqueous layer was extracted with EtOAc (50 mL). The organic extracts were combined, dried over Na₂SO₄, and concentrated to dryness under reduced pressure. The crude residue was purified by column chromatography (SiO₂, 0-60% EtOAc gradient in hexanes) to afford the desired product.

Step d: To a solution of the product from step c (6.0 g, 15.7 mmol, 1.0 equiv.) and urea (4.7 g, 78.7 mmol, 5 equiv) in EtOAc (52 mL, 0.3 M) was added AgOTf (10.0 g, 39.3 mmol, 2.5 equiv.). The resulting mixture was heated at 110° C. for 48 h in the dark. After completion, the reaction mixture was cooled to room temperature and diluted with water (100 mL) and EtOAc (100 mL). The resulting biphasic suspension was filtered through a Celite® pad. The organic phase was separated, and the aqueous layer was additionally extracted with EtOAc (50 mL). The combined organic extract was dried over Na₂SO₄ and concentrated to dryness under reduced pressure. The crude residue was purified via column chromatography (SiO₂, 0 to 20% MeOH gradient in dichloromethane) to afford the desired product.

Step e: To a solution of the product from step d (5.0 g, 14.5 mmol, 1.0 equiv.) in dioxane (50 mL) was added isoamyl nitrite (2.9 mL, 21.8 mmol, 1.5 equiv). The resulting mixture was stirred at 60° C. for 2 h. After completion, the solution was concentrated to dryness under reduced pressure and the crude residue was purified by column chromatography (SiO₂, 0-60% EtOAc gradient in hexanes) to afford the 4-[2-(2-chloro-6-cyclopropylpyridin-4-yl)-5-fluorophenyl]-5-methyl-1,3-oxazole as the desired product.

Step f: To a solution of 4-methoxy-5-(2-trimethylsilylethoxymethyl)pyrrolo[3,2-d]pyrimidine-6-carbaldehyde (0.70 g, 2.3 mmol, 1.0 equiv., prepared according to example 22) in acetonitrile (20 mL) and water (0.125 mL, 6.90 mmol, 3.0 equiv.), KI (611 mg, 3.68 mmol, 1.6 equiv.) was added, followed by TMSCl (0.47 mL, 3.68 mmol, 1.6 equiv.). The reaction mixture was stirred for 6 hours at 45° C. After cooling the mixture to room temperature, it was quenched with H₂O (30 mL) and extracted with EtOAc (3×35 mL). The combined organic extract was washed with brine, dried over Na₂SO₄ and concentrated to dryness under reduced pressure. The crude product was purified by column chromatography (SiO₂, 0 to 20% MeOH gradient in dichloromethane) to give the desired product.

Step g: To a solution of the product from step f (0.32 g, 1.1 mmol, 1.0 equiv.), 4-[2-(2-chloro-6-cyclopropylpyridin-4-yl)-5-fluorophenyl]-5-methyl-1,3-oxazole (0.36 g, 1.1 mmol, 1.0 equiv.), and K₂CO₃ (0.45 g, 3.3 mmol, 3.0 equiv.) in dioxane (25 mL) was added CuI (0.21 g, 1.1 mmol, 1.0 equiv.) and DMEDA (0.23 mL, 2.2 mmol, 2.0 equiv.). The reaction mixture was degassed by purging N₂ for 5 minutes and heated at 110° C. for 12 hours under vigorous stirring. The reaction mixture was cooled to room temperature, diluted with EtOAc (75 mL), and aq. sat. NaCl (25 mL). The organic phase was separated, and the aqueous layer was additionally extracted with EtOAc (2×30 mL). The combined organics were dried over Na₂SO₄, filtered, and concentrated to dryness under reduced pressure. The crude residue was purified via column chromatography (SiO₂, 0 to 100% EtOAc gradient in hexanes) to afford the desired product.

Step h: To a stirred solution of the product from step g (40 mg, 0.68 mmol, 1.0 equiv) and 2-methoxyethanamine (10.2 mg, 0.13 mmol, 2.0 equiv.) was added AcOH (7.8 mL, 0.13 mmol, 2.0 equiv.) and the mixture was stirred at room temperature for 20 mins. Then NaBH(OAc)₃ (28 mg, 0.13 mmol, 2.0 equiv.) was added, and the mixture was stirred at room temperature for 3 h. The reaction was quenched with aq. sat. NaHCO₃ (2 mL) and diluted with EtOAc (25 mL). The organic phase was separated, and the aqueous layer was additionally extracted with EtOAc (2×20 mL). The combined organic extract was dried over Na₂SO₄ and concentrated to dryness under reduced pressure. The crude residue was then treated with trifluoracetic acid/dichloromethane mixture (2 mL, 1:4 v/v) at room temperature for 3 h. After all volatiles were removed under vacuum the obtained crude material was then dissolved in NH₃ in methanol (7M, 3 mL). The resulting solution was stirred for 2 h before solvent evaporation. The crude product was purified by reversed phase preparative HPLC (20-90% CH₃CN in water with 0.1% formic acid) to afford the title compound. ¹H NMR (400 MHz, CDCl₃) δ 8.27 (s, 1H), 7.79 (s, 1H), 7.47 (dd, J=8.6, 5.6 Hz, 1H), 7.36 (d, J=1.4 Hz, 1H), 7.29 (d, J=2.7 Hz, 1H), 7.21-7.15 (m, 1H), 7.03 (d, J=1.4 Hz, 1H), 6.35 (d, J=0.9 Hz, 1H), 4.00 (s, 2H), 3.54-3.49 (m, 2H), 3.37 (s, 3H), 2.88-2.80 (m, 2H), 2.04-1.97 (m, 2H), 1.95 (s, 3H), 1.03-0.96 (m, 4H); ESI MS [M+H]⁺ for C₂₈H₂₇FN₆O₃, calcd 515.2, found 515.2.

### Example 79: 2-{[(S)-2-methoxy-1-methylethylamino]methyl}-6-{6-cyclopropyl-4-[4-fluoro-2-(5-methyl-1,3-oxazol-4-yl)phenyl]-2-pyridyl}-1,6-dihydro-1,4,6-triaza-7-indenone

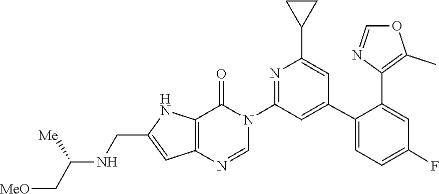 [SMILES: COC[C@H](C)NCc1cc2ncn(-c3cc(-c4ccc(F)cc4-c4ncoc4C)cc(C4CC4)n3)c(=O)c2[nH]1]

The title compound was prepared in a similar fashion to that described for example 78 using (2S)-1-methoxypropan-2-amine in step h. ¹H NMR (400 MHz, CDCl₃) δ 8.27 (s, 1H), 7.79 (s, 1H), 7.47 (dd, J=8.6, 5.7 Hz, 1H), 7.37 (d, J=1.3 Hz, 1H), 7.30-7.26 (m, 1H), 7.18 (td, J=8.3, 2.7 Hz, 1H), 7.02 (d, J=1.3 Hz, 1H), 6.33 (s, 1H), 4.08-3.93 (m, 2H), 3.41-3.36 (m, 4H), 3.30-3.23 (m, 1H), 3.01-2.91 (m, 1H), 2.04-1.98 (m, 1H), 1.95 (s, 3H), 1.06 (d, J=6.5 Hz, 3H), 1.01-0.96 (m, 4H); ESI MS [M+H]⁺ for C₂₉H₂₉FN₆O₃, calcd 529.2, found 529.2.

### Example 80: 6-{6-cyclopropyl-4-[4-fluoro-2-(5-methyl-1,3-oxazol-4-yl)phenyl]-2-pyridyl}-2-[(2-ethoxyethylamino)methyl]-1,6-dihydro-1,4,6-triaza-7-indenone

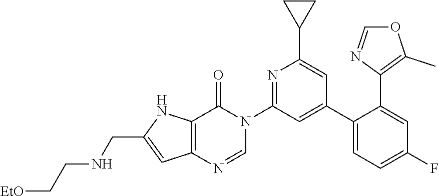 [SMILES: CCOCCNCc1cc2ncn(-c3cc(-c4ccc(F)cc4-c4ncoc4C)cc(C4CC4)n3)c(=O)c2[nH]1]

The title compound was prepared in a similar fashion to that described for example 78 using 2-ethoxyethylamine in step h. ¹H NMR (400 MHz, CDCl₃) δ 8.28 (s, 1H), 7.79 (s, 1H), 7.47 (dd, J=8.6, 5.6 Hz, 1H), 7.36 (d, J=1.4 Hz, 1H), 7.30-7.26 (m, 1H), 7.18 (td, J=8.3, 2.7 Hz, 1H), 7.02 (d, J=1.4 Hz, 1H), 6.37 (s, 1H), 4.03 (s, 2H), 3.61-3.49 (m, 4H), 2.90-2.76 (m, 2H), 2.07-1.90 (m, 4H), 1.22 (t, J=7.0 Hz, 3H), 1.03-0.93 (m, 4H). ESI MS [M+H]⁺ C₂₉H₂₉FN₆O₃, calcd 529.2, found 529.2.

### Example 81: 4-[2-(2-{[(S)-3-methyl-1-piperidyl]methyl}-4-cyclopropyl-7-oxo-1,6-dihydro-1,6-diaza-6-indenyl)-6-methyl-4-pyridyl]-3-(1-methyl-2-imidazolyl)benzonitrile

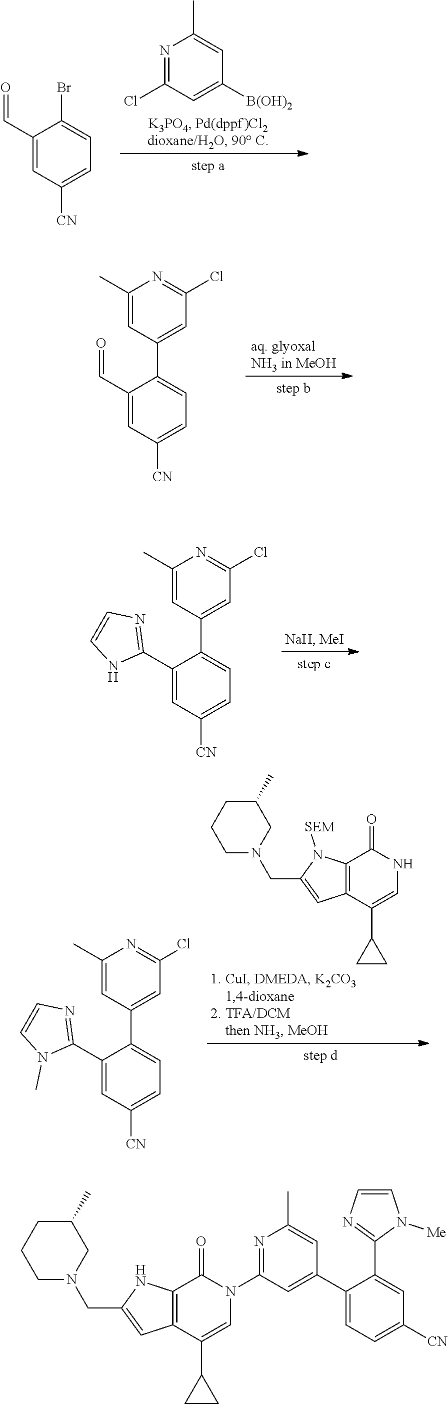

Step a: To a solution of 4-bromo-3-formylbenzonitrile (0.613 g, 2.92 mmol, 1.0 equiv.), (2-chloro-6-methylpyridin-4-yl)boronic acid (0.5 g, 2.92 mmol, 1.0 equiv.) and K₃PO₄ (1.86 g, 8.76 mmol, 3.0 equiv.) in dioxane/water mixture (6 mL, 5/1, v/v) was added Pd(dppf)Cl₂ (0.215 mg, 0.29 mmol, 0.1 equiv.). The resulting mixture was degassed by three vacuum/nitrogen backfill cycles and heated at 90° C. for 1 h. Once LCMS analysis indicated complete consumption of benzaldehyde starting material, the reaction was cooled to room temperature and partitioned between EtOAc (15 mL) and aqueous saturated NH₄Cl solution (15 mL). The organic phase was separated, and the aqueous layer was extracted with EtOAc (2×7 mL). The combined organic extract was dried over Na₂SO₄ and concentrated to dryness under reduced pressure. The crude product was purified by column chromatography (SiO₂, EtOAc in hexanes, 0 to 60%) to afford 4-(2-chloro-6-methylpyridin-4-yl)-3-formylbenzonitrile.

Step b: The product of step a (486 mg, 1.9 mmol, 1.0 equiv.) was mixed with aq. glyoxal (2.5 mL, 13.2 mmol, 7.0 equiv., 40 wt % solution) and NH₃ solution in MeOH (7M, 10.0 mL, 18.9 mmol, 10.0 equiv.). The obtained mixture was stirred at room temperature for 72 h before it was diluted with EtOAc (20 ml) and water (20 mL). The organic phase was separated, and the aqueous layer was extracted with EtOAc (2×10 mL). The combined organic extract was washed with water (25 mL), dried over Na₂SO₄, and the solvent was removed under reduced pressure. The crude product was purified by column chromatography (SiO₂, MeOH in dichloromethane, 0 to 10%) to afford 4-(2-chloro-6-methylpyridin-4-yl)-3-(1H-imidazol-2-yl)benzonitrile.

Step c: To a solution of the product of step b (180 mg, 0.61 mmol, 1.0 equiv.) in THE (5 mL, 0.12 M) was added NaH (74 mg, 1.84 mmol, 3.0 equiv., 60 wt % in mineral oil) at 0° C. The resulting suspension was stirred for 15 min before MeI (184 mg, 1.22 mmol, 2.0 equiv.) was added in one portion. The cooling bath was removed, and the reaction was stirred for 1 h at room temperature followed by quench with aqueous saturated NH₄Cl solution (3 mL). The reaction mixture was diluted with EtOAc (10 mL) and water (10 mL), the organic phase was separated, and the aqueous layer was extracted with EtOAc (2×5 mL). The combined organic phase was dried over Na₂SO₄, the solvent was evaporated under vacuum, and the crude residue was purified by column chromatography (SiO₂, MeOH in dichloromethane, 0 to 10%) to afford 4-(2-chloro-6-methylpyridin-4-yl)-3-(1-methylimidazol-2-yl)benzonitrile.

Step d: To a mixture of the product of step c (42 mg, 0.13 mmol, 1.0 equiv.), 4-cyclopropyl-2-[[(1-methylcyclobutyl)amino]methyl]-1-(2-trimethylsilylethoxymethyl)-6H-pyrrolo[2,3-c]pyridin-7-one (860 mg, 0.13 mmol, 1.0 equiv., prepared according to example 1) in dioxane (3.0 mL) was added CuI (26 mg, 0.13 mmol, 1.0 equiv.), N,N′-dimethylethylenediamine (50 mg, 0.54 mmol, 4.0 equiv.) and K₂CO₃ (57 mg, 0.40 mmol, 3.0 equiv.) The resulting mixture was purged with nitrogen for 10 min and heated at 110° C. overnight. The obtained material was partitioned between EtOAc (10 mL) and aqueous saturated NH₄Cl (10 mL). The organic layer was separated, and the aqueous layer was extracted with EtOAc (2×7 mL). The combined organic phase was washed with water (20 mL), dried over Na₂SO₄ and concentrated to dryness under reduced pressure. The crude coupling product was dissolved in trifluoroacetic acid/dichloromethane mixture (2 mL, 1:10 v/v), and the solution was stirred for 3 h at room temperature before all volatiles were removed under reduced pressure. The crude material was redissolved in NH₃ in methanol (7M, 2 mL), and the solution was stirred for 30 min before final solvents evaporation. The crude product was purified by prep-HPLC (C18 SiO₂, 10-100% CH₃CN in water with 0.1% formic acid) to afford the title compound. ¹H NMR (400 MHz, CDCl₃) δ 7.95 (d, J=1.7 Hz, 1H), 7.83 (dd, J=8.1, 1.7 Hz, 1H), 7.75 (s, 1H), 7.67 (d, J=8.1 Hz, 1H), 7.25 (s, 1H), 7.14 (d, J=1.2 Hz, 1H), 6.83 (d, J=1.2 Hz, 1H), 6.63 (d, J=1.4 Hz, 1H), 6.40 (s, 1H), 3.73 (d, J=2.5 Hz, 2H), 3.10 (s, 3H), 2.98-2.82 (m, 2H), 2.44 (s, 3H), 2.12-2.02 (m, 2H), 1.97-1.84 (m, 1H), 1.76 (d, J=8.1 Hz, 3H), 1.68 (d, J=8.2 Hz, 2H), 1.02-0.79 (m, 6H), 0.70 (td, J=5.9, 4.1 Hz, 2H). ESI MS [M+H]⁺ for C₃₄H₃₆N₇O, calcd 558.3, found 558.1.

### Example 82: 4-Cyclopropyl-6-[4-[4-fluoro-2-(1-methylimidazol-2-yl)phenyl]-6-methoxypyridin-2-yl]-2-[[(3S)-3-methylpiperidin-1-yl]methyl]-1H-pyrrolo[2,3-c]pyridin-7-one

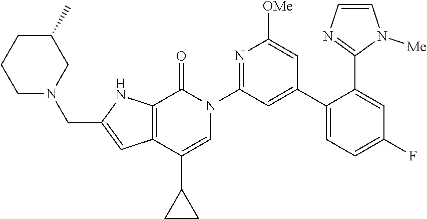 [SMILES: COc1cc(-c2ccc(F)cc2-c2nccn2C)cc(-n2cc(C3CC3)c3cc(CN4CCC[C@H](C)C4)[nH]c3c2=O)n1]

The title compounds was prepared in a similar fashion to example 81 starting from 4-cyclopropyl-2-[[(3S)-3-methylpiperidin-1-yl]methyl]-1,6-dihydropyrrolo[2,3-c]pyridin-7-one (prepared according to example 1) and 2-chloro-4-[4-fluoro-2-(1-methylimidazol-2-yl)phenyl]-6-methoxypyridine (prepared according to example 81 using (2-chloro-6-methoxypyridin-4-yl)boronic acid). ¹H NMR (400 MHz, CDCl₃) δ 9.73 (s, 1H), 7.55 (dd, J=8.6, 5.5 Hz, 1H), 7.44 (d, J=1.3 Hz, 1H), 7.33 (dd, J=8.9, 2.7 Hz, 1H), 7.27-7.18 (m, 2H), 7.12 (d, J=1.3 Hz, 1H), 6.86-6.80 (m, 1H), 6.36 (d, J=1.8 Hz, 1H), 6.27 (d, J=1.2 Hz, 1H), 3.85 (s, 3H), 3.59 (d, J=3.1 Hz, 2H), 3.15 (s, 3H), 2.76 (dd, J=17.7, 9.4 Hz, 2H), 1.97-1.81 (m, 2H), 1.77-1.45 (m, 5H), 0.93-0.77 (m, 6H), 0.73-0.62 (m, 2H). ESI MS [M+H]⁺ for C₃₃H₃₅FN₆O₂, calcd 567.3, found 567.2.

### Example 83: 4-Cyclopropyl-6-[6-cyclopropyl-4-[2-(1-methylimidazol-2-yl)-4-(trifluoromethyl)phenyl]pyridin-2-yl]-2-[[(3S)-3-methylpiperidin-1-yl]methyl]-1H-pyrrolo[2,3-c]pyridin-7-one

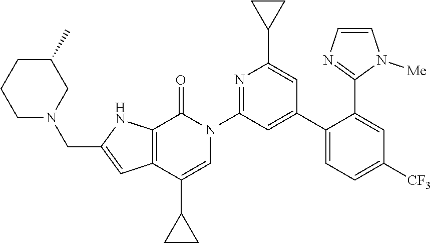 [SMILES: C[C@H]1CCCN(Cc2cc3c(C4CC4)cn(-c4cc(-c5ccc(C(F)(F)F)cc5-c5nccn5C)cc(C5CC5)n4)c(=O)c3[nH]2)C1]

The title compound was prepared in a similar fashion to example 13 starting from 4-cyclopropyl-2-[[(3S)-3-methylpiperidin-1-yl]methyl]-1,6-dihydropyrrolo[2,3-c]pyridin-7-one (prepared according to example 1) and 2-chloro-6-cyclopropyl-4-[2-(1-methylimidazol-2-yl)-4-(trifluoromethyl)phenyl]pyridine (prepared according to example 13 using 2-bromo-5-(trifluoromethyl)benzaldehyde). ¹H NMR (400 MHz, CD₃OD) δ 7.92-7.77 (m, 3H), 7.29 (d, J=1.4 Hz, 1H), 7.01 (d, J=1.3 Hz, 1H), 6.98 (d, J=1.3 Hz, 1H), 6.95 (d, J=1.2 Hz, 1H), 6.74 (d, J=1.4 Hz, 1H), 6.47 (s, 1H), 3.69 (s, 2H), 3.09 (s, 3H), 2.85 (dd, J=19.9, 11.1 Hz, 2H), 2.07-1.97 (m, 1H), 1.93 (tt, J=8.2, 4.8 Hz, 1H), 1.86 (dddd, J=13.5, 6.3, 5.2, 2.7 Hz, 1H), 1.76-1.45 (m, 5H), 0.90 (dt, J=8.0, 3.2 Hz, 2H), 0.86-0.76 (m, 8H), 0.60-0.54 (m, 2H). ESI MS [M+H]⁺ for C₃₆H₃₇F₃N₆O, calcd 627.3, found 627.2.

### Example 84: 2-{[(S)-3-methyl-1-piperidyl]methyl}-6-{4-[4-cyano-2-(1-methyl-2-imidazolyl)phenyl]-6-cyclopropyl-2-pyridyl}-7-oxo-1,6-dihydro-1,6-diaza-4-indenecarbonitrile

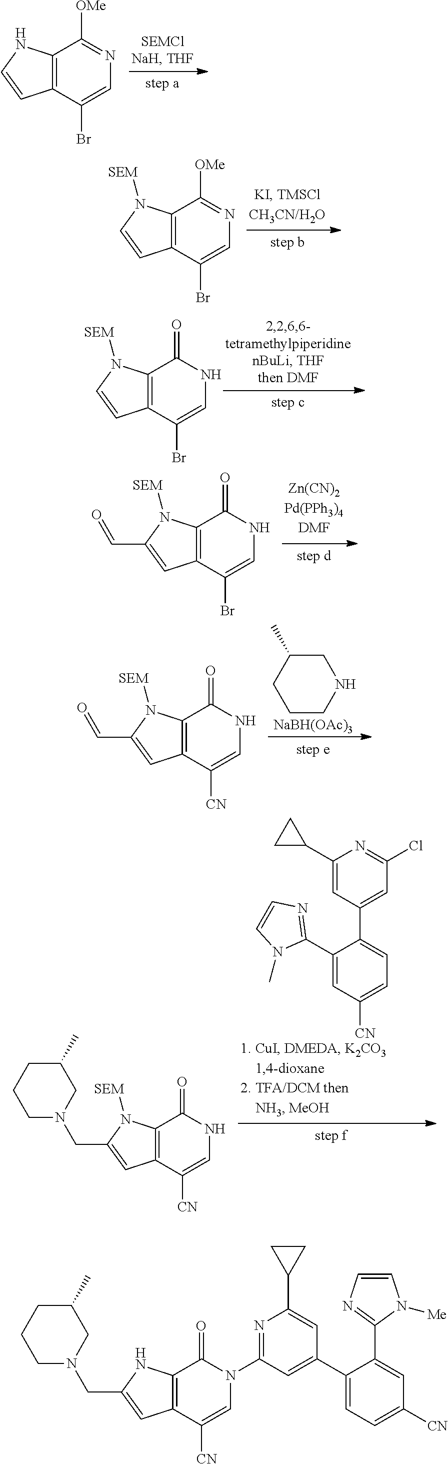

Step a: To a 1-L round bottom flask was added 4-bromo-7-methoxy-1H-pyrrolo[2,3-c]pyridine (10.0 g, 44.0 mmol) and THE (220 mL). The solution was cooled to 0° C., and NaH (60% in mineral oil, 1.94 g, 48.4 mmol) was added in portions over 10 min. The resulting mixture was stirred at 0° C. for 30 min followed by the addition of SEM-Cl (8.07 g, 48.4 mmol). The reaction was stirred at room temperature for 3.5 h. Once TLC analysis indicated full consumption of the starting material the reaction mixture was poured into saturated aqueous NH₄Cl (100 mL), and the product was extracted with EtOAc (3×100 mL). The combined organic extract was washed with water (100 mL), dried over MgSO₄ and concentrated to dryness under reduced pressure. The crude material was purified by column chromatography (SiO₂, EtOAc in hexanes. 0 to 15%) to furnish 2-[(4-bromo-7-methoxypyrrolo[2,3-c]pyridin-1-yl)methoxy]ethyl-trimethylsilane.

Step b: To a solution of the product from step a (14.6 g, 41.0 mmol) in acetonitrile (205 ml, 0.2 M) was added KI (7.5 g, 45.1 mmol) and water (60 μL). TMSCl (4.9 g, 45.1 mmol) was then added, and the reaction mixture was stirred at room temperature for 4.5 h. Once LCMS analysis indicated full conversion the reaction mixture was diluted with EtOAc (300 mL), washed with water (2×200 mL), dried over Na₂SO₄ and concentrated to dryness under reduced pressure. The crude product was purified by column chromatography (SiO₂, 0-90% EtOAc/hexanes) to furnish 4-bromo-1-(2-trimethylsilylethoxymethyl)-6H-pyrrolo[2,3-c]pyridin-7-one.

Step c: To a solution of 2,2,6,6-tetramethylpiperidine (1.2 mL, 7.0 mmol, 2.4 equiv.) in dry THE (10 mL) nBuLi (2.7 mL, 6.9 mmol, 2.35 equiv, 2.5 M solution in hexanes) was added dropwise at −78° C. under an atmosphere of nitrogen. After stirring for 5 min the resulting mixture was transferred to an ice bath and stirred for additional 15 min. The resulting solution of LiTMP was added dropwise to a solution of the product of step b (1.0 g, 2.9 mmol, 1 equiv.) in THE (20 mL) at −78° C. After the reaction was stirred at −78° C. for 1 h, DMF (0.7 mL, 8.7 mmol, 3 equiv.) was added dropwise over 1 min. The acetone/dry ice cooling bath was replaced with an ice bath, and the reaction mixture was stirred at 0° C. for 1 h. The resulting mixture was quenched by addition of aqueous saturated NH₄Cl (30 mL) followed by dilution with water (50 mL) and EtOAc (50 mL). The organic phase was separated, and the aqueous phase was extracted with EtOAc (3×30 mL). Combined organic extract was washed with brine (70 mL), dried over Na₂SO₄ and concentrated to dryness under reduced pressure. The dry residue was fractionated by column chromatography (SiO₂, EtOAc in dichloromethane, 0 to 80%) to produce 4-bromo-7-oxo-1-(2-trimethylsilylethoxymethyl)-6H-pyrrolo[2,3-c]pyridine-2-carbaldehyde.

Step d: A mixture of the bromide from step c (2.5 g 6.7 mmol, 1 equiv.), Zn(CN)₂ (0.8 g, 6.7 mmol, 1 equiv.), Pd(PPh₃)₄ (0.4 g, 0.34 mmol, 0.05 quiv.) and DMF (13.4 mL, 0.5M) was loaded in 40 mL vial equipped with a stirring bar. The reaction mixture was degassed by applying vacuum and backfilling with dry nitrogen 3 times followed by heating at 100° C. for 1.5 h. Once TLC analysis indicated complete consumption of the starting material the reaction was cooled to room temperature and partitioned between water (70 mL) and EtOAc (70 mL). The formed white precipitate was removed by filtration, and the organic layer was separated. The aqueous phase was additionally extracted with EtOAc (2×30 mL). The combined organic extract was washed with water and brine, dried over Na₂SO₄ and concentrated to dryness. The crude material was purified by column chromatography (SiO₂, EtOAc in hexanes, 0 to 90%) to yield 2-formyl-7-oxo-1-(2-trimethylsilylethoxymethyl)-6H-pyrrolo[2,3-c]pyridine-4-carbonitrile.

Step e: To the product of step d (0.2 g, 0.63 mmol, 1.0 equiv.) in dichloromethane (5 mL, 0.12 M) was added (S)-3-methylpiperidine hydrochloride (0.129 g, 0.945 mmol, 1.5 equiv.) and N,N-diisopropylethylamine (0.35 mL, 1.89 mmol, 3.0 equiv.). The resulting mixture was stirred at room temperature for 10 min before NaBH(OAc)₃ (0.28 g, 1.26 mmol, 2.0 equiv.) was added. The mixture was stirred at room temperature for 16 h. The reaction was quenched with aqueous saturated NaHCO₃ (5 mL) and diluted with dichloromethane (10 mL), the organic phase was separated, and the aqueous layer was extracted with dichloromethane (15 mL). The combined organic extract was dried over Na₂SO₄ and concentrated under reduced pressure. The crude residue was purified by column chromatography (SiO₂, MeOH in dichloromethane, 0 to 10%) to afford 2-[[(3S)-3-methylpiperidin-1-yl]methyl]-7-oxo-1-(2-trimethylsilylethoxymethyl)-6H-pyrrolo[2,3-c]pyridine-4-carbonitrile.

Step f: To a mixture of the product of step e (48 mg, 0.12 mmol, 1.0 equiv.), 4-(2-chloro-6-cyclopropylpyridin-4-yl)-3-(1-methylimidazol-2-yl)benzonitrile (40 mg, 0.12 mmol, 1.0 equiv., prepared according to example 13) in dioxane (3.0 mL) was added CuI (23 mg, 0.12 mmol, 1.0 equiv.), N,N′-dimethylethylenediamine (44 mg, 0.45 mmol, 4.0 equiv.) and K₂CO₃ (50 mg, 0.36 mmol, 3.0 equiv.) The resulting mixture was purged with nitrogen for 10 min and heated at 110° C. overnight. The reaction mixture was cooled to room temperature and partitioned between diluted with EtOAc (10 mL) and aqueous saturated NH₄Cl (10 mL). The organic phase was separated, and the aqueous phase was additionally extracted with EtOAc (10 mL). The combined organic extract was dried over Na₂SO₄ and concentrated to dryness under reduced pressure. The crude coupling product was dissolved in trifluoroacetic acid/dichloromethane mixture (2 mL, 1:10 v/v), and the solution was stirred for 3 h at room temperature before all volatiles were removed under reduced pressure. The crude material was redissolved in NH₃ in methanol (7M, 2 mL), and the solution was stirred for 30 min before final solvents evaporation. The crude product was purified by prep-HPLC (C18 SiO₂, 10-100% CH₃CN in water with 0.1% formic acid) to afford the title compound. ¹H NMR (400 MHz, CDCl₃) δ 8.13 (s, 1H), 7.95 (d, J=1.7 Hz, 1H), 7.85 (dd, J=8.0, 1.6 Hz, 1H), 7.75-7.61 (m, 2H), 7.16 (s, 1H), 6.86 (s, 1H), 6.65 (d, J=1.3 Hz, 1H), 6.42 (s, 1H), 3.81-3.65 (m, 2H), 3.10 (s, 3H), 2.86 (dd, J=27.9, 9.7 Hz, 2H), 2.10 (td, J=10.3, 9.4, 5.5 Hz, 1H), 1.98-1.88 (m, 1H), 1.85-1.59 (m, 5H), 0.99 (dt, J=8.0, 3.3 Hz, 2H), 0.88 (td, J=6.1, 3.7 Hz, 6H). ESI MS [M+H]⁺ for C₃₄H₃₃N₈O, calcd 569.3, found 569.1.

### Example 85: 6-[6-cyclopropyl-4-[4-fluoro-2-(4-methyl-1,2,4-triazol-3-yl)phenyl]pyridin-2-yl]-2-[[(2R)-2-methylmorpholin-4-yl]methyl]-7-oxo-1H-pyrrolo[2,3-c]pyridine-4-carbonitrile

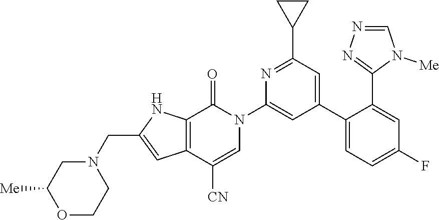 [SMILES: C[C@@H]1CN(Cc2cc3c(C#N)cn(-c4cc(-c5ccc(F)cc5-c5nncn5C)cc(C5CC5)n4)c(=O)c3[nH]2)CCO1]

The title compound was prepared in a similar fashion to that described for example 84 using (2R)-2-methylmorpholine for reductive amination step and 2-chloro-6-cyclopropyl-4-[4-fluoro-2-(4-methyl-1,2,4-triazol-3-yl)phenyl]pyridine (prepared according to example 28). ¹H NMR (400 MHz, DMSO-d₆) δ 12.56 (s, 1H), 8.46 (s, 1H), 8.21 (s, 1H), 7.72 (dd, J=8.7, 5.6 Hz, 1H), 7.66-7.51 (m, 2H), 7.23 (d, J=1.4 Hz, 1H), 6.92 (d, J=1.4 Hz, 1H), 6.32 (d, J=1.8 Hz, 1H), 3.69 (d, J=11.1 Hz, 1H), 3.59 (d, J=2.2 Hz, 2H), 3.51-3.41 (m, 2H), 3.23 (s, 3H), 2.72-2.59 (m, 2H), 2.08-1.98 (m, 2H), 1.72 (t, J=10.5 Hz, 1H), 0.99 (d, J=6.2 Hz, 3H), 0.97-0.90 (m, 2H), 0.88-0.81 (m, 2H). ESI MS [M+H]⁺ for C₃₁H₃₀FN₈O₂, calcd 565.3, found 565.3.

### Example 86: 6-{6-cyclopropyl-4-[4-fluoro-2-(1-methyl-2-imidazolyl)phenyl]-2-pyridyl}-2-[(3-fluoropropylamino)methyl]-7-oxo-1,6-dihydro-1,6-diaza-4-indenecarbonitrile

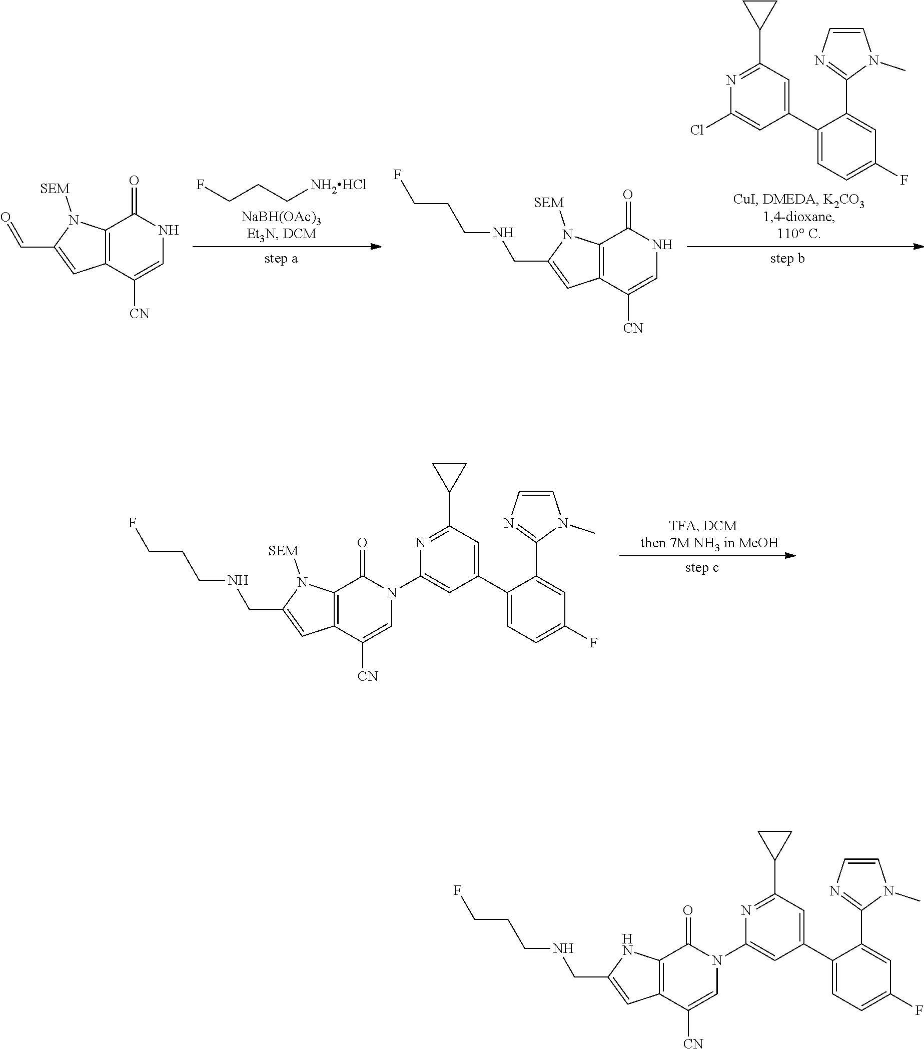

Step a: To a solution of 2-formyl-7-oxo-1-(2-trimethylsilylethoxymethyl)-6H-pyrrolo[2,3-c]pyridine-4-carbonitrile (0.159 g, 0.50 mmol, 1.0 equiv., prepared according to example 84) in dichloromethane (3.0 mL) was added 3-fluoropropan-1-amine hydrochloride (68.1 mg, 0.60 mmol, 1.2 equiv.) and triethylamine (0.14 mL, 1.0 mmol, 2.0 equiv.). The resulting mixture was stirred at room temperature for 10 min before NaBH(OAc)₃ (0.16 g, 0.75 mmol, 1.5 equiv.) was added. The mixture was stirred at room temperature for 16 h. The resulting solution was quenched with aq. sat. NaHCO₃ (2 mL) and diluted with dichloromethane. The organic phase was separated, and the aqueous layer was additionally extracted with dichloromethane (10 mL). The combined organic phase was dried over Na₂SO₄ and concentrated to dryness under vacuum. The crude residue was purified by column chromatography (SiO₂ C18, MeCN in H₂O, 10 to 60%, 0.1% formic acid) to afford 2-[(3-fluoropropylamino)methyl]-7-oxo-1-(2-trimethylsilylethoxymethyl)-6H-pyrrolo[2,3-c]pyridine-4-carbonitrile.

Step b: To a mixture of the product from step a (57 mg, 0.15 mmol, 1.0 equiv.) and 2-chloro-6-cyclopropyl-4-[4-fluoro-2-(1-methylimidazol-2-yl)phenyl]pyridine (49.2 mg, 0.15 mmol, 1.0 equiv., prepared according to example 51) in dioxane (1.5 mL, 0.1 M) was added CuI (28.6 mg, 0.15 mmol, 1.0 equiv.), DMEDA (26.4 mg, 0.30 mmol, 2.0 equiv.) and K₂CO₃ (62.2 mg, 0.45 mmol, 3.0 equiv.). The reaction was degassed by sparging with nitrogen for 5 min and heated at 100° C. overnight under vigorous stirring. The reaction was allowed to cool to 23° C. and diluted with aq. sat. NH₄Cl (3 mL) and EtOAc (10 mL). The organic phase was separated, and the aqueous layer was additionally extracted with EtOAc (2×5 mL). The combined organic phase was dried over Na₂SO₄, concentrated and the crude product was directly used for the next step without purification.

Step c: To a solution of the product from step b in dichloromethane (2 mL) was added trifluoracetic acid (1 mL). The resulting solution was stirred at 23° C. for 1 h. Upon volatiles evaporation under vacuum the residue was dissolved in 7M NH₃ in MeOH (3 mL). After 30 min of stirring at 23° C. the mixture was again concentrated under reduced pressure to dryness. The crude product was purified reversed phase prep-HPLC (C18 SiO₂, 10-100% CH₃CN in water with 0.1% formic acid) to furnish the title compound. ¹H NMR (400 MHz, CD₃OD) δ 8.07 (s, 1H), 7.75 (dd, J=8.7, 5.5 Hz, 1H), 7.44 (td, J=8.5, 2.8 Hz, 1H), 7.40-7.29 (m, 2H), 7.07 (d, J=1.3 Hz, 1H), 7.05 (d, J=1.4 Hz, 1H), 6.83 (d, J=1.4 Hz, 1H), 6.49 (s, 1H), 4.50 (dt, J=47.4, 5.8 Hz, 2H), 3.94 (s, 2H), 3.18 (s, 3H), 2.74 (t, J=7.2 Hz, 2H), 2.10-1.79 (m, 3H), 1.07-0.95 (m, 2H), 0.94-0.81 (m, 2H). ESI MS [M+H]⁺ for C₃₀H₂₇F₂N₇O, calcd 540.2, found 540.1.

### Example 87: 6-{6-cyclopropyl-4-[4-fluoro-2-(1-methyl-2-imidazolyl)phenyl]-2-pyridyl}-2-{(2,2-dioxo-2λ⁶-thia-6-aza-6-spiro[3.3]heptyl)methyl}-7-oxo-1,6-dihydro-1,6-diaza-4-indenecarbonitrile

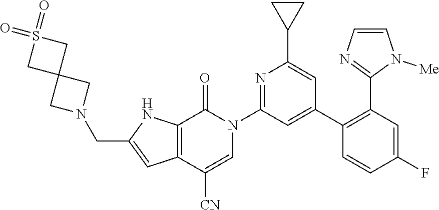 [SMILES: Cn1ccnc1-c1cc(F)ccc1-c1cc(C2CC2)nc(-n2cc(C#N)c3cc(CN4CC5(C4)CS(=O)(=O)C5)[nH]c3c2=O)c1]

The title compound was prepared in a similar fashion to that described for example 86 using 2λ⁶-6-thia-6-azaspiro[3.3]heptane 2,2-dioxide hydrochloride for the reductive amination step. ¹H NMR (400 MHz, CDCl₃) δ 10.02 (s, 1H), 8.05 (s, 1H), 7.54 (dd, J=8.6, 5.5 Hz, 1H), 7.51 (d, J=1.4 Hz, 1H), 7.36 (dd, J=8.8, 2.7 Hz, 1H), 7.32-7.24 (m, 1H), 7.13 (d, J=1.3 Hz, 1H), 6.84 (d, J=1.4 Hz, 1H), 6.68 (d, J=1.4 Hz, 1H), 6.39 (s, 1H), 4.24 (s, 4H), 3.74 (s, 2H), 3.46 (s, 4H), 3.11 (s, 3H), 1.94 (td, J=8.2, 4.1 Hz, 1H), 1.04-0.94 (m, 2H), 0.94-0.83 (m, 2H). ESI MS [M+H]⁺ for C₃₂H₂₈FN₇O₃S, calcd 610.2, found 610.2.

### Example 88: 2-{1[(S)-2-methyl-1,4-oxazepan-4-yl]methyl}-6-{6-cyclopropyl-4-[4,6-difluoro-2-(1-methyl-2-imidazolyl)phenyl]-2-pyridyl}-7-oxo-1,6-dihydro-1,6-diaza-4-indenecarbonitrile

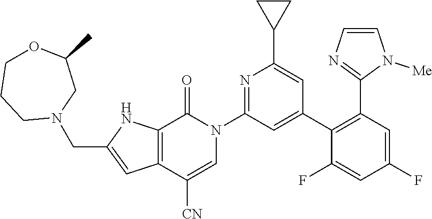 [SMILES: C[C@H]1CN(Cc2cc3c(C#N)cn(-c4cc(-c5c(F)cc(F)cc5-c5nccn5C)cc(C5CC5)n4)c(=O)c3[nH]2)CCCO1]

The title compound was prepared in a similar fashion to that described for example 86 using (S)-2-methyl-1,4-oxazepane for the reductive amination step and 2-chloro-6-cyclopropyl-4-[2,4-difluoro-6-(1-methylimidazol-2-yl)phenyl]pyridine (prepared according to example 51 using 2-bromo-3,5-difluorobenzaldehyde on step a). ¹H NMR (400 MHz, DMSO-d₆) δ 12.54 (s, 1H), 8.21 (s, 1H), 7.62 (ddd, J=10.3, 9.0, 2.6 Hz, 1H), 7.35 (ddd, J=8.8, 2.6, 1.2 Hz, 1H), 7.29 (t, J=1.5 Hz, 1H), 7.08 (d, J=1.2 Hz, 1H), 6.89 (d, J=1.2 Hz, 1H), 6.87 (d, J=1.2 Hz, 1H), 6.33 (s, 1H), 3.75 (s, 2H), 3.72-3.55 (m, 3H), 3.20 (s, 3H), 2.87-2.73 (m, 2H), 2.30-2.19 (m, 1H), 2.00 (td, J=8.2, 4.2 Hz, 1H), 1.87-1.73 (m, 1H), 1.75-1.62 (m, 1H), 1.04-0.89 (m, 5H), 0.89-0.77 (m, 2H). ESI MS [M+H]⁺ for C₃₃H₃₁F₂N₇O₂, calcd 596.3, found 596.2.

### Example 89: 2-{[(S)-2-methyl-1-piperidyl]methyl}-6-{6-cyclopropyl-4-[4,6-difluoro-2-(1-methyl-2-imidazolyl)phenyl]-2-pyridyl}-7-oxo-1,6-dihydro-1,6-diaza-4-indenecarbonitrile

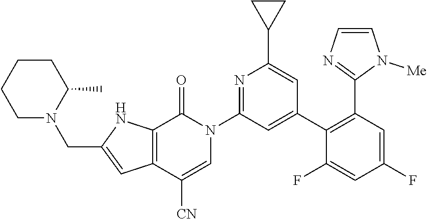 [SMILES: C[C@H]1CCCCN1Cc1cc2c(C#N)cn(-c3cc(-c4c(F)cc(F)cc4-c4nccn4C)cc(C4CC4)n3)c(=O)c2[nH]1]

The title compound was prepared in a similar fashion to that described for example 86 using (S)-2-methylpiperidine hydrochloride for the reductive amination step and 2-chloro-6-cyclopropyl-4-[2,4-difluoro-6-(1-methylimidazol-2-yl)phenyl]pyridine (prepared according to example 51 using 2-bromo-3,5-difluorobenzaldehyde on step a). ¹H NMR (400 MHz, DMSO-d₆) δ 12.51 (s, 1H), 8.20 (s, 1H), 7.62 (ddd, J=11.2, 9.1, 2.6 Hz, 1H), 7.35 (dt, J=8.4, 1.5 Hz, 1H), 7.29 (d, J=1.5 Hz, 1H), 7.08 (d, J=1.2 Hz, 1H), 6.89 (d, J=1.1 Hz, 1H), 6.87 (s, 1H), 6.27 (s, 1H), 3.84 (d, J=14.6 Hz, 1H), 3.55 (d, J=14.5 Hz, 1H), 3.20 (s, 3H), 2.72 (dt, J=11.4, 4.1 Hz, 1H), 2.28-2.16 (m, 1H), 2.06-1.90 (m, 2H), 1.60-1.29 (m, 4H), 1.24-1.14 (m, 2H), 1.09 (d, J=6.2 Hz, 3H), 0.98-0.88 (m, 2H), 0.88-0.79 (m, 2H). ESI MS [M+H]⁺ for C₃₃H₃₁F₂N₇O, calcd 580.3, found 580.2.

### Example 90: 6-{6-cyclopropyl-4-[4-fluoro-2-(4-methyl-4H-1,2,4-triazol-3-yl)phenyl]-2-pyridyl}-2-[hydroxy(tetrahydro-3-furyl)methyl]-7-oxo-1,6-dihydro-1,6-diaza-4-indenecarbonitrile

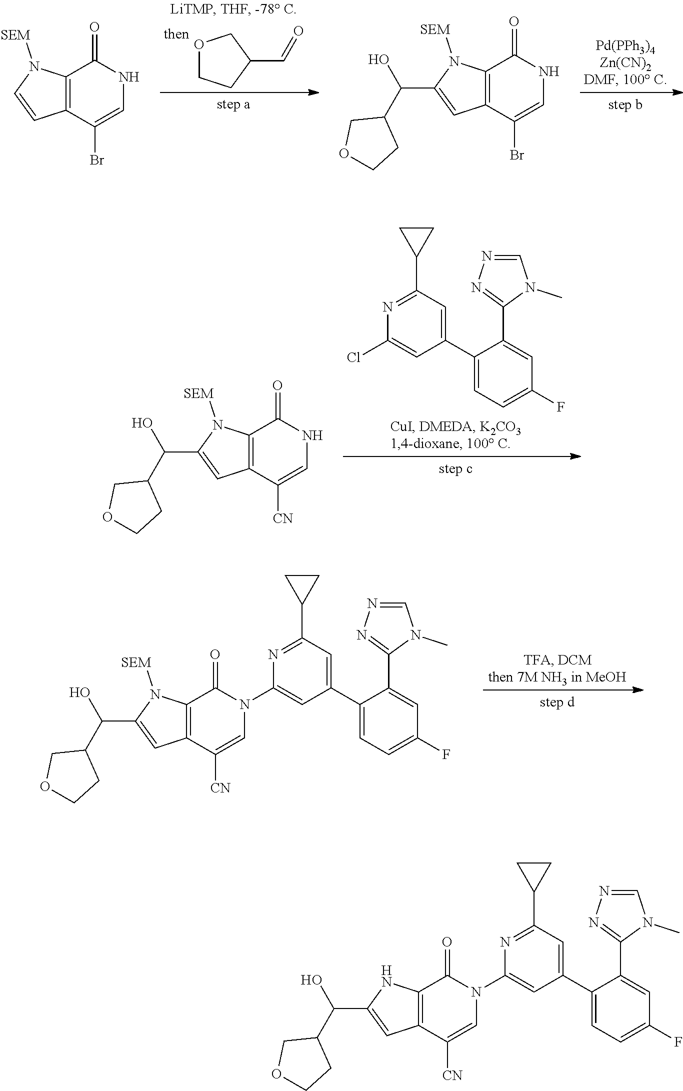

Step a: To a solution of 2,2,6,6-tetramethylpiperidine (720 mg, 5.10 mmol, 3.5 equiv.) in dry THE (14.6 mL) nBuLi (3.2 mL, 5.10 mmol, 3.5 equiv., 1.6M solution in hexanes) was added dropwise at −78° C. over 5 min under an atmosphere of nitrogen. The resulting mixture was stirred at −78° C. for 20 min before a solution of 4-bromo-1-(2-trimethylsilylethoxymethyl)-6H-pyrrolo[2,3-c]pyridin-7-one (500 mg, 1.46 mmol, 1.0 equiv., prepared according to example 84) in THE (3.3 mL) was added dropwise over 10 min. The obtained solution was stirred at −78° C. for 1.5 h followed by the addition of tetrahydrofuran-3-carbaldehyde (327 mg, 2.91 mmol, 2 equiv.). After 10 min the mixture was warmed up to 23° C. and stirred for additional 30 min. The resulting solution was diluted with aq. sat. NH₄Cl (5 mL) and water (15 mL), and the product was extracted with EtOAc (3×35 mL). The combined organic extract was dried over MgSO₄ and concentrated to dryness. The dry residue was fractionated by column chromatography (SiO₂, 0 to 80% EtOAc gradient in hexanes,) to produce the desired product.

Step b: To a solution of the product from step a (420 mg, 0.95 mmol, 1.0 equiv.) in DMF (9.5 mL) under N₂ was added Zn(CN)₂ (376 mg, 3.21 mmol, 3.5 equiv.) and Pd(PPh₃)₄ (110 mg, 0.3 mmol, 0.3 equiv.). The resulting mixture was heated at 100° C. for 2 days. After cooling down to room temperature, the reaction mixture was diluted with EtOAc (50 mL) and water (50 mL) and filtered through a Celite® pad. The organic phase was separated and the aqueous phase was additionally extracted with EtOAc (2×25 mL). The combined organic extract was sequentially washed with water (100 mL) and brine (100 mL), dried over MgSO₄ and concentrated to dryness. The crude residue was purified by reverse phase column chromatography (C18 column, 0 to 100% MeCN gradient in water with 0.1% formic acid) to afford the desired product.

Step c: To a mixture of the product from step b (75 mg, 0.19 mmol, 1.0 equiv.) and 2-chloro-6-cyclopropyl-4-[4-fluoro-2-(4-methyl-1,2,4-triazol-3-yl)phenyl]pyridine (63 mg, 0.19 mmol, 1.0 equiv., prepared according example 28) in dioxane (3.8 mL, 0.05 M) was added CuI (36 mg, 0.19 mmol, 1.0 equiv.), DMEDA (34 mg, 0.38 mmol, 2.0 equiv.) and K₂CO₃ (79 mg, 0.57 mmol, 3.0 equiv.). The resulting mixture was sparged with nitrogen for 10 min, the reaction vial was sealed and heated at 100° C. for 16 h under vigorous stirring. The resulting suspension was allowed to cool to 23° C., diluted with aq. sat. NH₄Cl (5 mL) and EtOAc (20 mL). The organic phase was separated, and the aqueous layer was additionally extracted with EtOAc (2×10 mL). The combined organic phase was dried over MgSO₄, concentrated and the crude residue was purified by reverse phase column chromatography (C18 column, 0 to 100% MeCN gradient in water) to provide corresponding coupling product.

Step d: The product from step c (134 mg, 0.20 mmol, 1.0 equiv.) was treated with the mixture of trifluoroacetic acid and dichloromethane (2 mL, 1:1 v/v) at 0° C. for 2 h. The obtained solution was concentrated under reduced pressure, and the residue was redissolved in 7M NH₃ in methanol (2 mL) at 0° C. After 30 min at 0° C. all volatiles were evaporated under vacuum, and the crude product was fractionated by reverse phase column chromatography (SiO₂ C18 column, 0 to 100% CH₃CN gradient in water with 0.1% formic acid) to furnish the title compound. ¹H NMR (400 MHz, CDCl₃) δ 11.44 (d, J=49.3 Hz, 1H), 8.22 (s, 1H), 7.96 (d, J=3.2 Hz, 1H), 7.65-7.59 (m, 1H), 7.42-7.31 (m, 2H), 7.29-7.25 (m, 1H), 7.12-7.06 (m, 1H), 6.25 (s, 1H), 3.79 (d, J=7.4 Hz, 2H), 3.72-3.58 (m, 2H), 3.55 (s, 1H), 3.14 (s, 3H), 2.65 (s, 1H), 2.03 (d, J=5.5 Hz, 2H), 1.95-1.85 (m, 1H), 1.08-0.98 (m, 4H). ESI MS [M+H]⁺ for C₃₀H₂₇FN₇O₃, calcd 552.2, found 552.1.

### Example 91: 2-(cyclopentylhydroxymethyl)-6-{6-cyclopropyl-4-[4-fluoro-2-(4-methyl-4H-1,2,4-triazol-3-yl)phenyl]-2-pyridyl}-7-oxo-1,6-dihydro-1,6-diaza-4-indenecarbonitrile

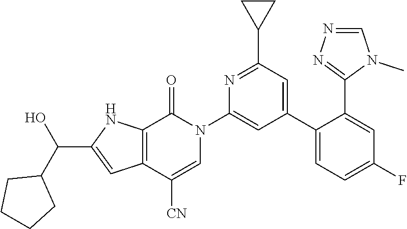 [SMILES: Cn1cnnc1-c1cc(F)ccc1-c1cc(C2CC2)nc(-n2cc(C#N)c3cc(C(O)C4CCCC4)[nH]c3c2=O)c1]

The title compound was prepared according to the protocol described for example 90 using cyclopentanecarbaldehyde on step a. ¹H NMR (400 MHz, CDCl₃) δ 11.33 (s, 1H), 8.20 (s, 1H), 7.96 (d, J=0.8 Hz, 1H), 7.61 (dd, J=8.6, 5.3 Hz, 1H), 7.40-7.30 (m, 3H), 7.02 (s, 1H), 6.26 (s, 1H), 4.62 (d, J=7.6 Hz, 1H), 3.13 (s, 3H), 2.20 (d, J=8.1 Hz, 1H), 2.02-1.96 (m, 1H), 1.81 (s, 2H), 1.57-1.35 (m, 6H), 1.18 (s, 1H), 1.05-0.93 (m, 4H). ESI MS [M+H]⁺ for C₃₁H₂₉FN₇O₂, calcd 550.2, found 550.2.

### Example 92: 6-{6-Cyclopropyl-4-[4-fluoro-2-(4-methyl-4H-1,2,4-triazol-3-yl)phenyl]-2-pyridyl}-2-[hydroxy(3-pyridyl)methyl]-7-oxo-1,6-dihydro-1,6-diaza-4-indenecarbonitrile

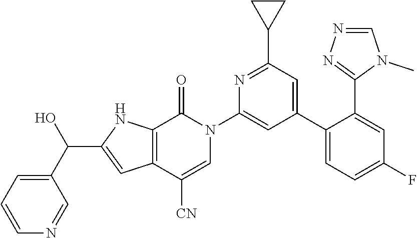 [SMILES: Cn1cnnc1-c1cc(F)ccc1-c1cc(C2CC2)nc(-n2cc(C#N)c3cc(C(O)c4cccnc4)[nH]c3c2=O)c1]

The title compound was prepared according to the protocol described for example 90 using pyridine-3-carboxaldehyde on step a. ¹H NMR (400 MHz, CDCl₃) δ 12.21 (s, 1H), 8.41 (s, 2H), 8.21 (s, 1H), 7.92 (s, 1H), 7.60 (s, 2H), 7.39-7.29 (m, 2H), 7.12 (s, 2H), 5.96 (s, 2H), 3.17 (s, 3H), 2.05 (s, 2H), 1.05 (d, J=8.2 Hz, 4H). ESI MS [M+H]⁺ for C₃₁H₂₅FN₈O₂, calcd 559.2, found 559.1.

### Example 93: 6-{6-cyclopropyl-4-[4-fluoro-2-(4-methyl-4H-1,2,4-triazol-3-yl)phenyl]-2-pyridyl}-2-[hydroxy(6-methyl-2-pyridyl)methyl]-7-oxo-1,6-dihydro-1,6-diaza-4-indenecarbonitrile

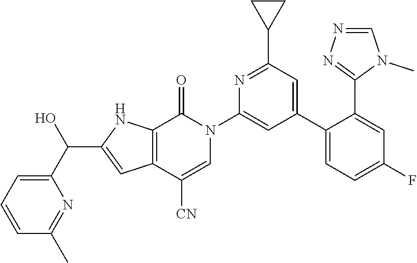 [SMILES: Cc1cccc(C(O)c2cc3c(C#N)cn(-c4cc(-c5ccc(F)cc5-c5nncn5C)cc(C5CC5)n4)c(=O)c3[nH]2)n1]

The title compound was prepared according to the protocol described for example 90 using 6-methylpyridine-2-carbaldehyde on step a. ¹H NMR (400 MHz, CDCl₃) δ 10.30 (s, 1H), 8.12 (s, 1H), 8.02 (s, 1H), 7.65-7.53 (m, 2H), 7.43-7.31 (m, 3H), 7.14 (dd, J=10.0, 7.7 Hz, 2H), 6.92 (d, J=1.4 Hz, 1H), 6.43 (d, J=1.8 Hz, 1H), 5.92 (s, 1H), 3.14 (s, 3H), 2.57 (s, 3H), 2.02-1.91 (m, 1H), 1.06-0.89 (m, 4H). ESI MS [M+H]⁺ for C₃₂H₂₅FN₈O₂, calcd 573.2, found 573.1.

### Example 94: 2-[Amino(3-pyridyl)methyl]-6-{6-cyclopropyl-4-[4-fluoro-2-(4-methyl-4H-1,2,4-triazol-3-yl)phenyl]-2-pyridyl}-7-oxo-1,6-dihydro-1,6-diaza-4-indenecarbonitrile

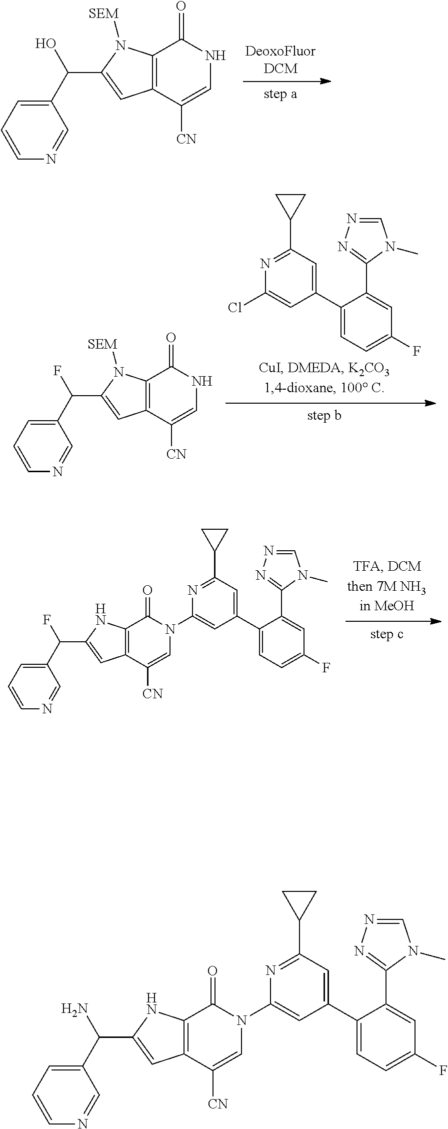 [SMILES: C[Si](C)(C)CCOCn1c(C(O)c2cccnc2)cc2c(C#N)c[nH]c(=O)c21 ; C[Si](C)(C)CCOCn1c(C(F)c2cccnc2)cc2c(C#N)c[nH]c(=O)c21 ; Cn1cnnc1-c1cc(F)ccc1-c1cc(Cl)nc(C2CC2)c1 ; Cn1cnnc1-c1cc(F)ccc1-c1cc(C2CC2)nc(-n2cc(C#N)c3cc(C(F)c4cccnc4)[nH]c3c2=O)c1 ; Cn1cnnc1-c1cc(F)ccc1-c1cc(C2CC2)nc(-n2cc(C#N)c3cc(C(N)c4cccnc4)[nH]c3c2=O)c1]

Step a: Deoxo-Fluor® (0.34 gm 0.75 mmol, 1.5 equiv, 50 wt % solution in toluene) was added dropwise to a solution of 2-[hydroxy(pyridin-3-yl)methyl]-1-(2-trimethylsilylethoxymethyl)-6H-pyrrolo[2,3-c]pyridin-7-one (0.2 g, 0.5 mmol, 1 equiv., prepared similarly to example 90 using pyridine-3-carbaldehyde) in dichloromethane (2.5 mL, 0.2 M) at 0° C. over 5 min. The resulting yellow mixture was stirred at 0° C. for 20 min. Once TLC analysis indicated complete consumption of the starting material the mixture was carefully quenched with aq. sat. NaHCO₃ (3 mL), and the product was extracted with dichloromethane (3×7 mL). The combined organic extract was dried over Na₂SO₄ and concentrated to dryness under reduced pressure. The crude residue was fractionated by column chromatography (SiO₂, 0 to 70% EtOAc gradient in dichlorormethane) to afford the desired fluorinated product.

Step b, c: Steps b and c were performed in an analogous fashion to that described for steps c and d of example 90 to afford the title compound. ¹H NMR (400 MHz, CDCl₃) δ 11.42 (s, 1H), 8.64 (d, J=55.4 Hz, 2H), 8.07 (s, 1H), 8.01 (d, J=1.1 Hz, 1H), 7.72 (d, J=7.8 Hz, 1H), 7.58 (dd, J=8.4, 5.4 Hz, 1H), 7.44-7.28 (m, 4H), 7.01 (s, 1H), 6.27 (s, 1H), 5.50 (s, 2H), 3.12 (d, J=1.1 Hz, 3H), 2.00 (td, J=8.0, 4.1 Hz, 2H), 1.06-0.94 (m, 4H). ESI MS [M+H]⁺ for C₃₁H₂₆FN₉O, calcd 558.2, found 558.1.

### Example 95: 2-[amino(6-methyl-2-pyridyl)methyl]-6-{6-cyclopropyl-4-[4-fluoro-2-(4-methyl-4H-1,2,4-triazol-3-yl)phenyl]-2-pyridyl}-7-oxo-1,6-dihydro-1,6-diaza-4-indenecarbonitrile

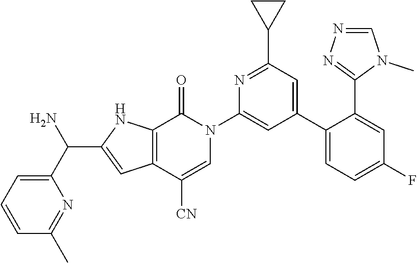 [SMILES: Cc1cccc(C(N)c2cc3c(C#N)cn(-c4cc(-c5ccc(F)cc5-c5nncn5C)cc(C5CC5)n4)c(=O)c3[nH]2)n1]

The title compound was prepared in similar fashion to example 94 using 2-chloro-6-cyclopropyl-4-[4-fluoro-2-(4-methyl-1,2,4-triazol-3-yl)phenyl]pyridine (prepared according to example 28). ¹H NMR (400 MHz, CDCl3) δ 8.12 (s, 1H), 8.02 (s, 1H), 7.63-7.55 (m, 2H), 7.46 (d, J=1.4 Hz, 1H), 7.43-7.32 (m, 2H), 7.12 (dd, J=16.1, 7.7 Hz, 2H), 6.89 (d, J=1.2 Hz, 1H), 6.31 (s, 1H), 5.35 (d, J=21.7 Hz, 1H), 3.15 (s, 3H), 2.57 (s, 3H), 1.95 (tt, J=8.1, 4.8 Hz, 1H), 1.28-1.25 (m, 2H), 1.03-0.86 (m, 5H). ESI MS [M+H]+ for C32H27FN9O, calcd 572.2, found 572.2.

### Example 96: 6-{6-cyclopropyl-4-[4-fluoro-2-(4-methyl-4H-1,2,4-triazol-3-yl)phenyl]-2-pyridyl}-2-[(1-hydroxycyclohexyl)methyl]-7-oxo-1,6-dihydro-1,6-diaza-4-indenecarbonitrile

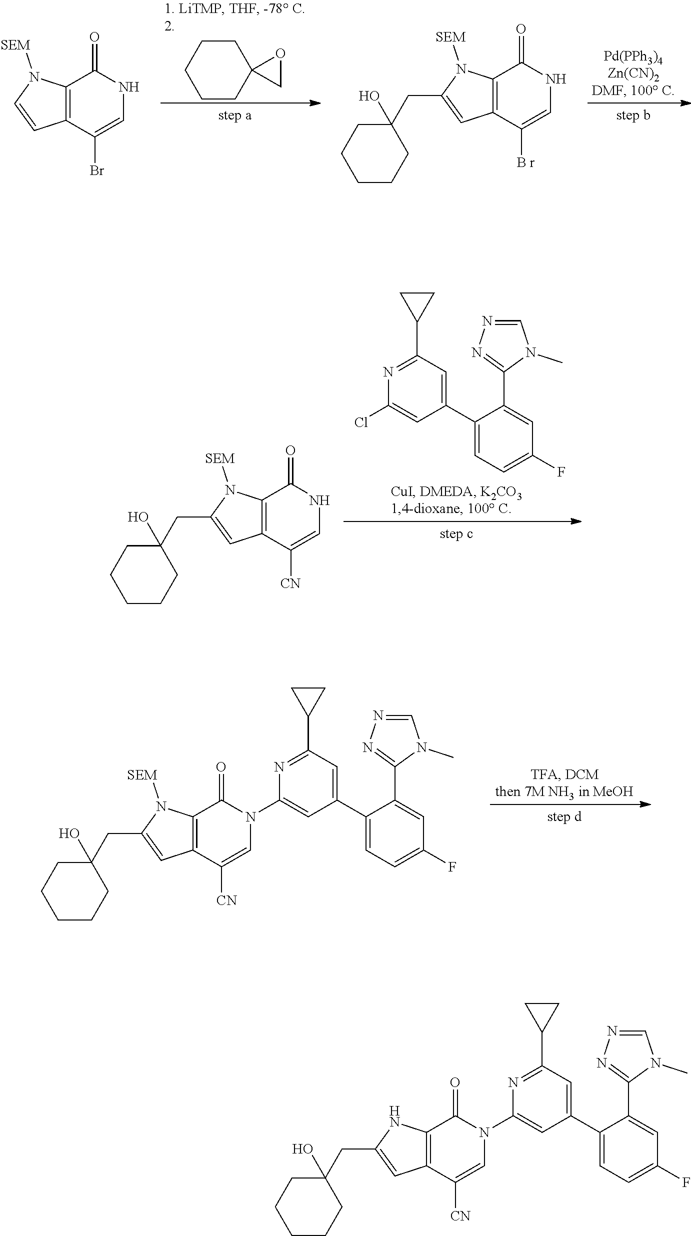

Step a: To a solution of 2,2,6,6-tetramethylpiperidine (0.86 mL, 5.10 mmol, 3.5 equiv.) in dry THE (14.6 mL), nBuLi (3.2 mL, 5.10 mmol, 3.5 equiv., 1.6 M solution in hexanes) was added dropwise at −78° C. over 5 min under an atmosphere of nitrogen. The resulting mixture was stirred at −78° C. for 20 min before a solution of 4-bromo-1-(2-trimethylsilylethoxymethyl)-6H-pyrrolo[2,3-c]pyridin-7-one (500 mg, 1.46 mmol, 1.0 equiv., prepared according to example 84) in THE (3.3 mL) was added dropwise over 10 min. The obtained solution was stirred at −78° C. for 1.5 h followed by the addition of 1-oxaspiro[2.5]octane (0.25 g, 2.23 mmol, 1.5 equiv.). After 10 min the mixture was warmed up to 23° C. and stirred for additional 30 min. The resulting solution was diluted with aq. sat. NH₄Cl (5 mL) and water (15 mL), and the product was extracted with EtOAc (3×35 mL). The combined organic extract was dried over Na₂SO₄ and concentrated to dryness. The dry residue was fractionated by reversed phase column chromatography (SiO₂ C18, 0 to 100% CH₃CN gradient in water with 0.1% formic acid) to produce the desired product.

Step b: To a solution of the product from step a (180 mg, 0.4 mmol, 1.0 equiv.) in DMF (0.8 mL) under N₂ was added Zn(CN)₂ (46 mg, 0.4 mmol, 1.0 equiv.) and Pd(PPh₃)₄ (46 mg, 0.04 mmol, 0.1 equiv.). The resulting mixture was heated at 100° C. for 2 days. After cooling down to room temperature, the reaction mixture was diluted with EtOAc (10 mL) and water (10 mL) and filtered through a Celite® pad. The organic phase was separated, and the aqueous phase was additionally extracted with EtOAc (2×7 mL). The combined organic extract was sequentially washed with water (10 mL) and brine (10 mL), dried over Na₂SO₄ and concentrated to dryness. The crude residue was purified by reverse phase column chromatography (C18 column, 0 to 100% MeCN gradient in water with 0.1% formic acid) to afford the desired product.

Step c: To a mixture of the product from step b (50 mg, 0.13 mmol, 1.0 equiv.) and 2-chloro-6-cyclopropyl-4-[4-fluoro-2-(4-methyl-1,2,4-triazol-3-yl)phenyl]pyridine (41 mg, 0.13 mmol, 1.0 equiv., prepared according to example 28) in dioxane (2.5 mL, 0.05 M) was added CuI (24 mg, 0.13 mmol, 1.0 equiv.), DMEDA (22 mg, 0.25 mmol, 2.0 equiv.) and K₂CO₃ (52 mg, 0.38 mmol, 3.0 equiv.). The resulting mixture was sparged with nitrogen for 10 min and heated at 100° C. under nitrogen for 16 h under vigorous stirring. The reaction was allowed to cool to 23° C., diluted with aq. sat. NH₄Cl (2 mL) and EtOAc (10 mL). The organic phase was separated, and the aqueous layer was additionally extracted with EtOAc (10 mL). The combined organic phase was dried over MgSO₄ and concentrated under reduced pressure to dryness. The crude residue was purified by column chromatography (0 to 20% MeOH gradient in dichlorormethane) to afford corresponding coupling product.

Step d: The product from step c was treated with a mixture of trifluoroacetic acid and dichloromethane (0.6 mL, 1:1 v/v) at 0° C. for 45 min. The obtained solution was concentrated under reduced pressure, and the residue was redissolved in 7M NH₃ in methanol (0.7 mL). The mixture was stirred for 45 min at 0° C. Upon concentration to dryness the residue was fractionated by reversed phase prep-HPLC (C18 column, 10 to 100% MeCN gradient in water with 0.1% formic acid) to furnish the title compound. ¹H NMR (400 MHz, CDCl₃) δ 10.80 (s, 1H), 8.19 (s, 1H), 7.99 (s, 1H), 7.61 (dd, J=8.6, 5.4 Hz, 1H), 7.45-7.30 (m, 3H), 6.92 (d, J=1.3 Hz, 1H), 6.30 (s, 1H), 3.14 (s, 3H), 2.87 (s, 2H), 2.01-1.94 (m, 1H), 1.60-1.40 (m, 9H), 1.32 (d, J=9.9 Hz, 1H), 1.02 (ddt, J=8.1, 5.8, 2.9 Hz, 2H), 0.98-0.92 (m, 2H). ESI MS [M+H]⁺ for C₃₂H₃₁FN₇O₂, calcd 564.2, found 564.2.

### Example 97: 2-[(R)-1-amino-2,2,2-trifluoroethyl]-6-{6-cyclopropyl-4-[4-fluoro-2-(4-methyl-4H-1,2,4-triazol-3-yl)phenyl]-2-pyridyl}-7-oxo-1,6-dihydro-1,6-diaza-4-indenecarbonitrile

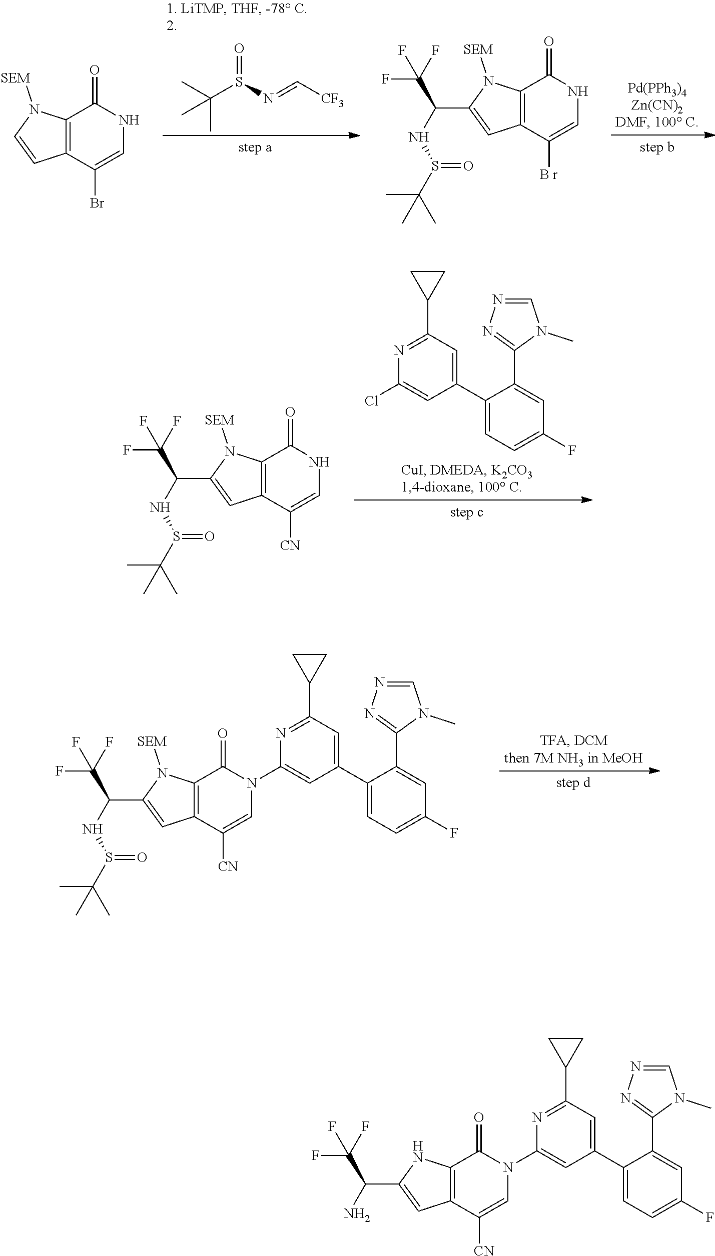

Step a: To a solution of 2,2,6,6-tetramethylpiperidine (1.7 g, 12.25 mmol, 3.5 equiv.) in dry THF (35.0 mL), nBuLi (7.6 mL, 12.25 mmol, 3.5 equiv, 1.6 M solution in hexanes) was added dropwise over 5 min at −78° C. under an atmosphere of nitrogen and stirred for 1 h. Then a solution of 4-bromo-1-(2-trimethylsilylethoxymethyl)-6H-pyrrolo[2,3-c]pyridin-7-one (1.2 g, 3.50 mmol, 1.0 equiv., prepared according to example 84) in THE (35.0 mL) was added dropwise over 10 min. The reaction mixture was stirred at −78° C. for 2 h followed by the addition of (R,E)-2-methyl-N-(2,2,2-trifluoroethylidene)propane-2-sulfinamide (1.4 g, 7.01 mmol, 2 equiv.). Then acetone/dry ice bath was replaced with an ice bath, and the reaction mixture was stirred at 0° C. for 1.5 h. The resulting mixture was quenched by addition of aq. sat. NH₄Cl (10 mL) and diluted with water (30 mL) and EtOAc (30 mL). The organic phase was separated, and the aqueous phase was extracted with EtOAc (2×20 mL). The combined organic extract was dried over MgSO₄ and concentrated to dryness. The dry residue was fractionated by column chromatography (SiO₂, 20-100% EtOAc gradient in hexanes) to produce (R)—N-[(1R)-1-[4-bromo-7-oxo-1-(2-trimethylsilylethoxymethyl)-6H-pyrrolo[2,3-c]pyridin-2-yl]-2,2,2-trifluoroethyl]-2-methylpropane-2-sulfinamide.

Step b: To a solution of the product from step a (987 mg, 1.82 mmol, 1.0 equiv.) in DMF (18.2 mL) under N₂ was added Zn(CN)₂ (640 mg, 5.45 mmol, 3.0 equiv.) and Pd(PPh₃)₄ (525 mg, 0.45 mmol, 0.25 equiv.). The resulting mixture was heated at 100° C. overnight. After cooling down to room temperature, the reaction mixture was diluted with EtOAc (70 mL) and water (70 mL). The resulting mixture was filtered through a Celite® pad, and the organic phase was separated. The aqueous layer was additionally extracted with EtOAc (2×35 mL). Combined organic extract was washed with water (2×100 mL) and brine (100 mL), dried over MgSO₄ and concentrated to dryness. The crude product was purified by reverse phase column chromatography (SiO₂ C18 column, 0 to 100% MeCN gradient in water with 0.1% formic acid) to afford the desired cyanation product.

Step c: To a mixture of the product from step b (184 mg, 0.48 mmol, 1.0 equiv.) and 2-chloro-6-cyclopropyl-4-[4-fluoro-2-(4-methyl-1,2,4-triazol-3-yl)phenyl]pyridine (157 mg, 0.48 mmol, 1.0 equiv., prepared according to example 28) in dioxane (9.6 mL, 0.05 M) was added CuI (91 mg, 0.48 mmol, 1.0 equiv.), DMEDA (84 mg, 0.96 mmol, 2.0 equiv.) and K₂CO₃ (198 mg, 1.43 mmol, 3.0 equiv.). The resulting mixture was sparged with nitrogen for 10 min and heated at 100° C. under N₂ for 16 h under vigorous stirring. The reaction was allowed to cool to 23° C. and diluted with aq. sat. NH₄Cl (2 mL) and EtOAc (10 mL). The organic phase was separated, and the aqueous layer was additionally extracted with EtOAc (2×5 mL). The combined organic phase was dried over MgSO₄, concentrated and the crude residue was purified by column chromatography (SiO₂, 0 to 20% MeOH gradient in dichlorormethane) to provide corresponding coupling product.

Step d: The product from step c (20 mg, 0.03 mmol, 1.0 equiv.) was treated with the mixture of trifluoroacetic acid and dichloromethane (0.5 mL, 1:1 v/v) at 0° C. for 2 h. The obtained solution was concentrated under reduced pressure, and the residue was redissolved in 7M NH₃ in methanol (2 mL) at 0° C. After 30 min at 0° C. all volatiles were evaporated under vacuum, and the crude product was fractionated by reverse phase column chromatography (SiO₂ C18 column, 0 to 100% CH₃CN gradient in water with 0.1% formic acid) to furnish the title compound. ¹H NMR (400 MHz, CDCl₃) δ 10.90 (s, 1H), 8.15 (s, 1H), 8.03 (s, 1H), 7.57 (dd, J=8.6, 5.4 Hz, 1H), 7.44-7.31 (m, 3H), 7.00 (d, J=1.2 Hz, 1H), 6.59 (s, 1H), 4.70 (d, J=6.5 Hz, 1H), 3.14 (s, 3H), 2.13 (d, J=40.5 Hz, 2H), 1.98 (td, J=8.1, 4.0 Hz, 1H), 1.02 (ddt, J=8.2, 5.7, 2.7 Hz, 2H), 0.96 (dt, J=5.2, 2.8 Hz, 2H). ESI MS [M+H]⁺ for C₂₇H₂₁F₄N₈O, calcd 549.2, found 549.1.

### Example 98: 2-[(S)-1-amino-2,2,2-trifluoroethyl]-6-{6-cyclopropyl-4-[4-fluoro-2-(4-methyl-4H-1,2,4-triazol-3-yl)phenyl]-2-pyridyl}-7-oxo-1,6-dihydro-1,6-diaza-4-indenecarbonitrile

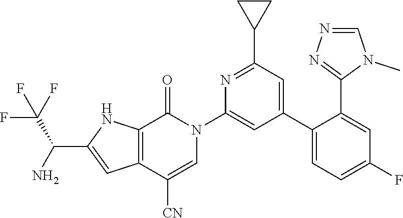 [SMILES: Cn1cnnc1-c1cc(F)ccc1-c1cc(C2CC2)nc(-n2cc(C#N)c3cc([C@H](N)C(F)(F)F)[nH]c3c2=O)c1]

The title compound was prepared in similar fashion to example 97 using (S,E)-2-methyl-N-(2,2,2-trifluoroethylidene)propane-2-sulfinamide on step a. ¹H NMR (400 MHz, CDCl₃) δ 10.90 (s, 1H), 8.15 (s, 1H), 8.03 (s, 1H), 7.57 (dd, J=8.6, 5.4 Hz, 1H), 7.44-7.31 (m, 3H), 7.00 (d, J=1.2 Hz, 1H), 6.59 (s, 1H), 4.70 (d, J=6.5 Hz, 1H), 3.14 (s, 3H), 2.13 (d, J=40.5 Hz, 2H), 1.98 (td, J=8.1, 4.0 Hz, 1H), 1.02 (ddt, J=8.2, 5.7, 2.7 Hz, 2H), 0.96 (dt, J=5.2, 2.8 Hz, 2H). ESI MS [M+H]⁺ for C₂₇H₂₁F₄N₈O, calcd 549.2, found 549.1.

### Example 99: 4-[2-(2-{[(S)-3-methyl-1-piperidyl]methyl}-4-mesyl-7-oxo-1,6-dihydro-1,6-diaza-6-indenyl)-6-cyclopropyl-4-pyridyl]-3-(1-methyl-2-imidazolyl)benzonitrile

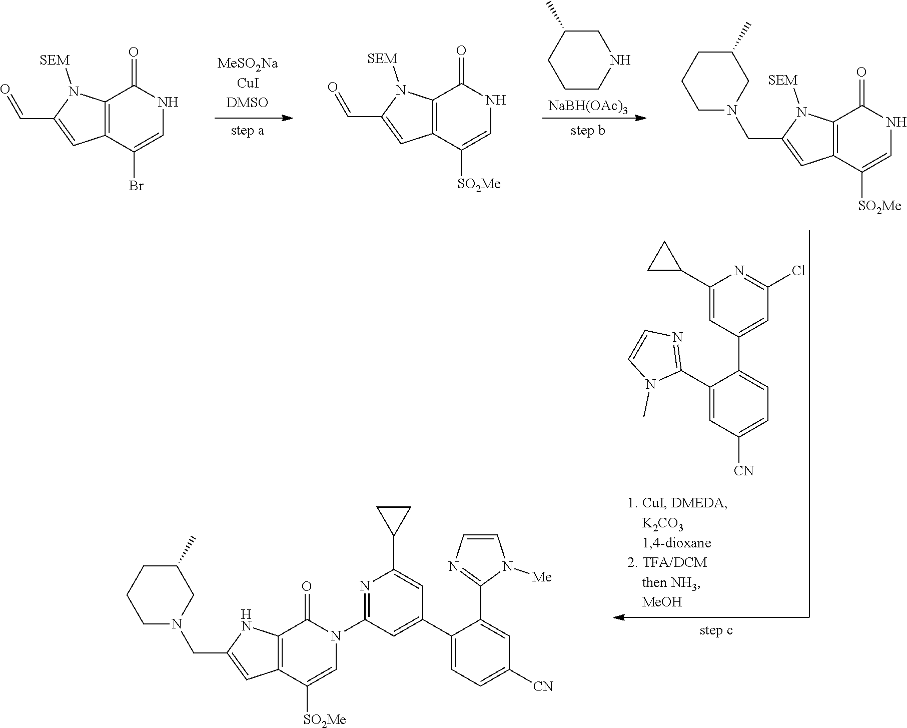

Step a. A mixture of 4-bromo-7-oxo-1-(2-trimethylsilylethoxymethyl)-6H-pyrrolo[2,3-c]pyridine-2-carbaldehyde (2.5 g, 6.7 mmol, 1 equiv., prepared according to example XX 84), sodium methanesulfonate (2.1 g, 20.2 mmol, 3 equiv.) and CuI (3.8 g, 20.2 mmol, 3 equiv.) in DMSO (34 mL, 0.2 M) was degassed by three cycles of vacuum/backfilling with nitrogen and heated at 100° C. for 24 h. The resulting mixture was cooled to room temperature, diluted with EtOAc (100 mL) and poured in water (100 mL). The resulting white precipitate was removed by filtration through a Celite® pad. The filtrate was transferred to a separatory funnel, the organic phase was separated and the aqueous phase was additionally extracted with EtOAc (2×30 mL). The combined organic phase was washed with water (2×100 mL), dried over Na₂SO₄ and concentrated to dryness under reduced pressure. The crude product was purified by column chromatography (SiO2, 0-80% EtOAc in dichloromethane) to afford 4-methylsulfonyl-7-oxo-1-(2-trimethylsilylethoxymethyl)-6H-pyrrolo[2,3-c]pyridine-2-carbaldehyde.

Step b: To the solution of product from step a (0.2 g, 0.44 mmol, 1.0 equiv.) in dichloromethane (5 mL, 0.1 M) was added (S)-3-methylpiperidine hydrochloride (0.1 g, 0.66 mmol, 1.5 equiv.) and N,N-diisopropylethylamine (0.2 mL, 1.1 mmol, 3.0 equiv.). The resulting mixture was stirred at 23° C. for 10 min before NaBH(OAc)₃ (0.2 g, 0.88 mmol, 2.0 equiv.) was added. The resulting mixture was stirred for 16 h at 23° C. The reaction was diluted with dichloromethane (10 mL) and quenched with aq. sat. NaHCO₃ (10 mL). The organic phase was separated, and the aqueous layer was extracted with dichloromethane (10 mL). The combined organic phase was dried over Na₂SO₄, concentrated under reduced pressure, and the crude residue was purified by column chromatography (SiO₂, MeOH in dichloromethane, 0 to 10%) to afford 2-[[(3S)-3-methylpiperidin-1-yl]methyl]-4-methylsulfonyl-1-(2-trimethylsilylethoxymethyl)-6H-pyrrolo[2,3-c]pyridin-7-one.

Step c: To a mixture of the product of step b (50 mg, 0.11 mmol, 1.0 equiv.), 4-(2-chloro-6-cyclopropylpyridin-4-yl)-3-(1-methylimidazol-2-yl)benzonitrile (37 mg, 0.119 mmol, 1.0 equiv., prepared according to example 13) in dioxane (3.0 mL) was added CuI (21 mg, 0.11 mmol, 1.0 equiv.), N,N′-dimethylethylenediamine (39 mg, 0.44 mmol, 4.0 equiv.) and K₂CO₃ (46 mg, 0.33 mmol, 3.0 equiv.) The resulting mixture was sparged with nitrogen for 10 min and heated at 110° C. overnight. The reaction mixture was cooled to room temperature and partitioned between EtOAc (10 mL) and aq. sat. NH₄Cl (10 mL). The organic phase was separated, and the aqueous phase was extracted with EtOAc (10 mL). The combined organic extract was dried over Na₂SO₄ and concentrated to dryness under reduced pressure. The crude coupling product was dissolved in trifluoroacetic acid/dichloromethane mixture (2 mL, 1:10 v/v) and stirred at 23° C. for 3 h. Upon solvents evaporation under vacuum the dry residue was redissolved in NH₃ in methanol (7M, 2 mL). After 30 min the solvent was evaporated, and the product was purified by prep-HPLC (SiO₂ C18, 10 to 90% MeCN in water with 0.1% trifluoroacetic acid) to afford the title compound. ¹H NMR (400 MHz, CDCl₃) δ 8.32 (s, 1H), 7.96 (d, J=1.7 Hz, 1H), 7.86 (dd, J=8.1, 1.6 Hz, 1H), 7.69 (d, J=8.0 Hz, 1H), 7.65 (d, J=1.2 Hz, 1H), 7.16 (s, 1H), 6.87 (s, 1H), 6.64 (d, J=1.3 Hz, 1H), 6.59 (s, 1H), 3.89-3.65 (m, 2H), 3.19 (s, 3H), 3.08 (s, 3H), 2.96 (s, 1H), 2.87 (d, J=14.2 Hz, 1H), 2.10 (d, J=12.0 Hz, 2H), 1.95 (tt, J=8.4, 4.8 Hz, 1H), 1.79 (t, J=10.5 Hz, 3H), 1.70 (s, 2H), 0.98 (dt, J=8.0, 3.3 Hz, 2H), 0.92-0.80 (m, 6H). ESI MS [M+H]⁺ for C₃₄H₃₆N₇O₃S, calcd 622.3, found 622.1.

### Example 100: 4-[2-(2-{1-[(S)-3-methyl-1-piperidyl]ethyl}-4-cyclopropyl-7-oxo-1,6-dihydro-1,6-diaza-6-indenyl)-6-cyclopropyl-4-pyridyl]-3-(1-methyl-2-imidazolyl)benzonitrile

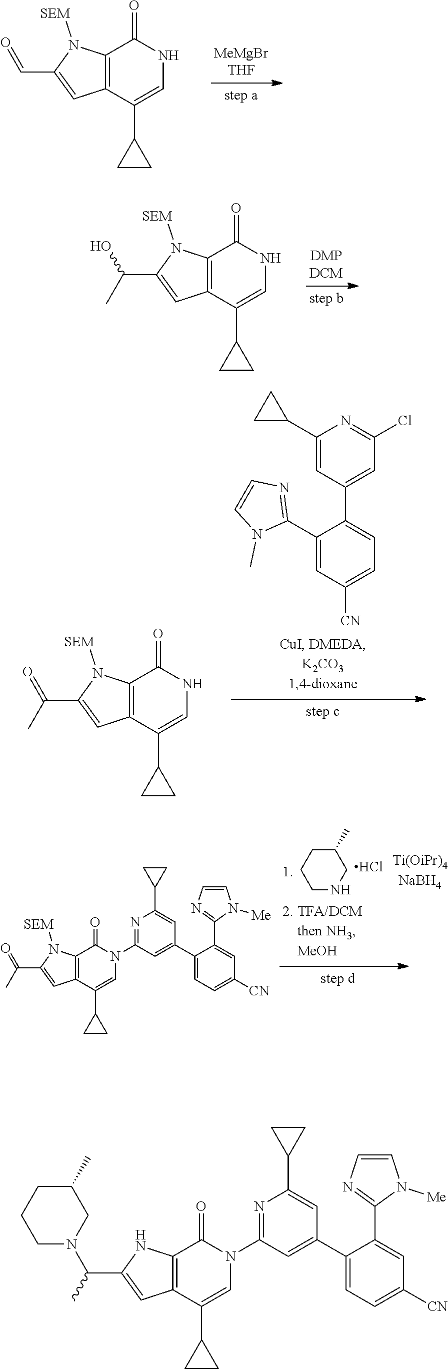

Step a: To the solution of 4-cyclopropyl-7-oxo-1-(2-trimethylsilylethoxymethyl)-6H-pyrrolo[2,3-c]pyridine-2-carbaldehyde (0.5 g, 1.5 mmol, 1.0 equiv., prepared according to example 1) in THE (10 mL, 0.15 M) was added MeMgBr (2 ml, 3.0 mmol, 4.0 equiv., 3 M in THF) at 0° C. The mixture was stirred at room temperature for 2 h before being quenched with aq. sat. NH₄Cl (5 mL). The mixture was diluted with EtOAc (15 ml), the organic phase was separated, and the aqueous layer was additionally extracted with EtOAc (2×10 mL). The combined organic phase was dried over Na₂SO₄ and concentrated to dryness under reduced pressure. The crude product was purified by column chromatography (SiO₂, MeOH in dichloromethane, 0 to 10%) to afford 4-cyclopropyl-2-(1-hydroxyethyl)-1-(2-trimethylsilylethoxymethyl)-6H-pyrrolo[2,3-c]pyridin-7-one.

Step b: To a mixture of the product of step a (520 mg, 1.5 mmol, 1.0 equiv.) and NaHCO₃ (630 mg, 7.5 mmol, 5.0 equiv.) in dichloromethane (20 mL) was added DMP (1.6 g, 3.75 mmol, 2.5 equiv.) at 0° C. After 10 min the cooling bath was removed, and the reaction was stirred at 23° C. for 2 h. The resulting mixture was carefully quenched with aq. Na₂S₂O₃ (15 mL) and aq. sat. NaHCO₃ (15 mL). After the biphasic solution was vigorously stirred for 1 h the organic phase was separated, and the aqueous layer was extracted with dichloromethane (2×20 mL). The combined organic extract was dried over Na₂SO₄, and the solvent was evaporated under reduced pressure. The crude oxidation product was purified by column chromatography (SiO₂, EtOAc in dichloromethane, 0 to 90%) to afford 2-acetyl-4-cyclopropyl-1-(2-trimethylsilylethoxymethyl)-6H-pyrrolo[2,3-c]pyridin-7-one.

Step c: To a mixture of the product of step b (100 mg, 0.29 mmol, 1.0 equiv), 4-(2-chloro-6-cyclopropylpyridin-4-yl)-3-[4-(difluoromethyl)-1,2,4-triazol-3-yl]benzonitrile (96 mg, 0.29 mmol, 1.0 equiv., prepared according to example 13) in dioxane (3.0 mL) was added CuI (55 mg, 0.29 mmol, 1.0 equiv.), N,N′-dimethylethylenediamine (100 mg, 1.15 mmol, 4.0 equiv.) and K₂CO₃ (120 mg, 0.87 mmol, 3.0 equiv.) The reaction mixture was sparged with nitrogen for 10 min and heated at 110° C. overnight. The obtained suspension was partitioned between aq. sat. NH₄Cl (5 mL) and EtOAc (15 mL). The organic phase was separated, and the aqueous layer was extracted with EtOAc (15 mL). The combined organic phase was dried over Na₂SO₄, the solvent was evaporated under reduced pressure, and the crude residue was purified by column chromatography (SiO₂, MeOH in dichloromethane, 0 to 10%) to afford 4-[2-[2-acetyl-4-cyclopropyl-7-oxo-1-(2-trimethylsilylethoxymethyl)pyrrolo[2,3-c]pyridin-6-yl]-6-cyclopropylpyridin-4-yl]-3-(1-methylimidazol-2-yl)benzonitrile.

Step d: To the solution of the product from step c (72 mg, 0.11 mmol, 1.0 equiv.) in THE (3 mL, 0.03 M) was added (S)-3-methylpiperidine hydrochloride (23 mg, 0.17 mmol, 1.5 equiv.) and Ti(OiPr)₄ (0.17 mL, 0.56 mmol, 5.0 equiv.). The reaction mixture was stirred at 60° C. for 1 h and cooled to room temperature followed by the addition of NaBH₄ (21 mg, 0.56 mmol, 5.0 equiv.) and stirring overnight. The resulting solution was diluted with EtOAc (20 mL), sequentially washed with H₂O (10 mL) and brine (10 mL), filtered through Na₂SO₄ and concentrated to dryness under vacuum. The crude amination product was dissolved in trifluoroacetic acid/dichloromethane mixture (2 mL, 1:10 v/v) and stirred at 23° C. for 3 h. Upon solvents evaporation under vacuum the dry residue was redissolved in NH₃ in methanol (7M, 2 mL). After 30 min the solvent was evaporated, and the product was purified by prep-HPLC (SiO₂ C18, 10 to 90% MeCN in water with 0.1% trifluoroacetic acid) to afford the title compound as an inseparable mixture of diastereoisomers in 2:1 ratio. ¹H NMR (400 MHz, CDCl₃, mixture of diastereomers) δ 7.94 (dd, J=4.4, 1.7 Hz, 1H), 7.91-7.76 (m, 2H), 7.64 (dd, J=20.2, 8.1 Hz, 1H), 7.29 (t, J=1.0 Hz, 1H), 7.16 (d, J=1.3 Hz, 1H), 6.93-6.63 (m, 1H), 6.48 (d, J=1.4 Hz, 1H), 6.35 (s, 1H), 3.96 (s, 1H), 3.06 (d, J=15.7 Hz, 3H), 2.79 (dt, J=31.5, 17.0 Hz, 2H), 2.28 (d, J=11.0 Hz, 2H), 2.04-1.85 (m, 3H), 1.74 (m, 4H), 1.50 (d, J=6.7 Hz, 3H), 1.14-0.77 (m, 9H), 0.75-0.61 (m, 2H). ESI MS [M+H]⁺ for C₃₇H₄₀N₇O, calcd 598.3, found 598.1.

### Example 101: 4-[2-(2-{1-[(S)-3-methyl-1-piperidyl]ethyl}-4-mesyl-7-oxo-1,6-dihydro-1,6-diaza-6-indenyl)-6-cyclopropyl-4-pyridyl]-3-(1-methyl-2-imidazolyl)benzonitrile

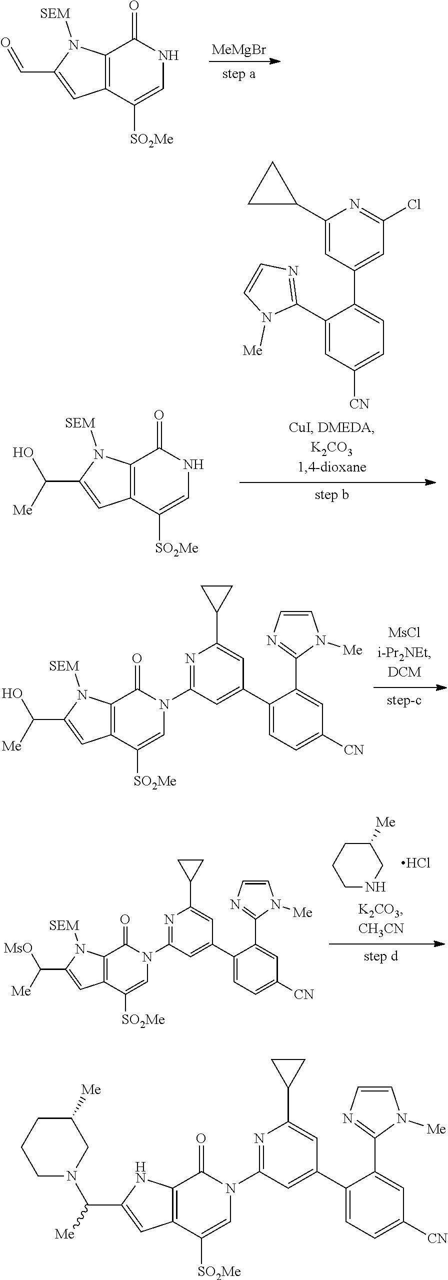

Step a: To the solution of 4-methylsulfonyl-7-oxo-1-(2-trimethylsilylethoxymethyl)-6H-pyrrolo[2,3-c]pyridine-2-carbaldehyde (0.2 g, 0.44 mmol, 1.0 equiv., prepared according to example 99 in THE (5 mL, 0.15 M) was added MeMgBr (0.6 ml, 1.76 mmol, 4.0 equiv., 3 M in THF) at 0° C. The resulting mixture was stirred at 0° C. for 2 h, quenched with aq. sat. NH₄Cl (2 mL) and diluted with EtOAc (15 mL) and water (10 mL). The organic phase was separated, and the aqueous layer was additionally extracted with EtOAc (2×10 mL). The combined organic phase was dried over Na₂SO₄, concentrated under reduced pressure, and the crude product was purified by column chromatography (SiO₂, MeOH in dichloromethane, 0 to 10%) to afford 2-(1-hydroxyethyl)-4-methylsulfonyl-1-(2-trimethylsilylethoxymethyl)-6H-pyrrolo[2,3-c]pyridin-7-one.

Step b: To a mixture of the product of step a (152 mg, 0.40 mmol, 1.0 equiv), 4-(2-chloro-6-cyclopropylpyridin-4-yl)-3-(1-methylimidazol-2-yl)benzonitrile (132 mg, 0.40 mmol, 1.0 equiv., prepared according to example 13) in dioxane (5.0 mL, 0.08 M) was added CuI (75 mg, 0.40 mmol, 1.0 equiv.), N,N′-dimethylethylenediamine (140 mg, 1.60 mmol, 4.0 equiv.) and K₂CO₃ (164 mg, 1.20 mmol, 3.0 equiv.). The resulting mixture was sparged with nitrogen for 10 min and heated at 110° C. overnight. The obtained suspension was partitioned between EtOAc (15 mL) and aq. sat. NH₄Cl (10 mL). The organic phase was separated, and the aqueous phase was extracted with EtOAc (2×5 mL). The combined extract was dried over Na₂SO₄, and the solvent was removed under reduced pressure. The crude coupling product was purified by column chromatography (SiO₂, MeOH in dichlorormethane, 0 to 10%) to afford 4-[2-cyclopropyl-6-[2-(1-hydroxyethyl)-4-methylsulfonyl-7-oxo-1-(2-trimethylsilyl-ethoxymethyl)pyrrolo[2,3-c]pyridin-6-yl]pyridin-4-yl]-3-(1-methylimidazol-2-yl)benzonitrile.

Step c: To the solution of the product of step b (140 mg, 0.20 mmol, 1.0 equiv.) in dichlorormethane (3 mL, 0.07 M) were sequentially added N,N-diisopropylethylamine (0.07 mL, 0.41 mmol, 2.0 equiv.) and MsCl (47 mg, 0.17 mmol, 1.5 equiv.) at 0° C. The cooling bath was removed, and the reaction was stirred at 23° C. for 1 h. Once TLC analysis indicated complete transformation the mixture was diluted with dichloromethane (15 mL) and sequentially washed with water (10 mL) and brine (10 mL). The organic extract was dried over Na₂SO₄ and concentrated to dryness to afford crude 1-[6-[4-[4-cyano-2-(1-methylimidazol-2-yl)phenyl]-6-cyclopropylpyridin-2-yl]-4-methylsulfonyl-7-oxo-1-(2-trimethylsilylethoxy-methyl)pyrrolo[2,3-c]pyridin-2-yl]ethyl methanesulfonate which was used in the next step without further purification.

Step d: To a solution of the product of step c (78 mg, 0.10 mmol, 1.0 equiv.) in acetonitrile (3 mL, 0.03 M) were added (S)-3-methylpiperidine hydrochloride (30 mg, 0.20 mmol, 2.0 equiv.) and K₂CO₃ (30 mg, 0.56 mmol, 5.0 equiv.). The obtained suspension was heated at 90° C. overnight. Once cooled to 23° C. the mixture was diluted with EtOAc (15 mL), then sequentially washed with H₂O (10 mL) and brine (10 mL). The organic phase was separated, dried over Na₂SO₄ and concentrated to dryness under reduced pressure. The crude amination product was purified by prep-HPLC (SiO₂ C18, 10 to 90% MeCN in water with 0.1% trifluoroacetic acid) to afford the title compound as a mixture of inseparable diastereomers in 1:1 ratio. ¹H NMR (400 MHz, CDCl₃, mixture of diatereomers) δ 8.31 (s, 1H), 7.96 (d, J=1.7 Hz, 1H), 7.86 (dd, J=8.1, 1.7 Hz, 1H), 7.75-7.60 (m, 2H), 7.16 (d, J=1.3 Hz, 1H), 6.87 (d, J=1.2 Hz, 1H), 6.63 (d, J=1.4 Hz, 1H), 6.56 (s, 1H), 3.97 (s, 1H), 3.20 (s, 3H), 3.08 (s, 3H), 2.79 (m, 2H), 2.02 (m, 2H), 1.94 (m, 2H), 1.79 (s, 5H), 1.50 (m, 2H), 0.98 (dt, J=8.1, 3.3 Hz, 2H), 0.95-0.79 (m, 5H). ESI MS [M+H]⁺ for C₃₅H₃₈N₇O₃S, calcd 636.3, found 636.1.

### Example 102: 4-[2-(2-{1-[(R)-2-methyl-4-morpholinyl]ethyl}-4-mesyl-7-oxo-1,6-dihydro-1,6-diaza-6-indenyl)-6-cyclopropyl-4-pyridyl]-3-(1-methyl-2-imidazolyl)benzonitrile

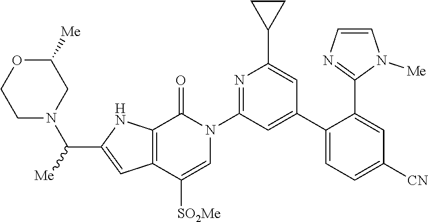 [SMILES: CC(c1cc2c(S(C)(=O)=O)cn(-c3cc(-c4ccc(C#N)cc4-c4nccn4C)cc(C4CC4)n3)c(=O)c2[nH]1)N1CCO[C@H](C)C1]

The title compound was prepared as a mixture of diastereomers (1:1 ratio) in a similar fashion to that described for example 101 starting from aldehyde described in example 99. ¹H NMR (400 MHz, CDCl₃, mixture of diastereomers) δ 8.28 (s, 1H), 7.96 (d, J=1.7 Hz, 1H), 7.86 (dd, J=8.1, 1.8 Hz, 1H), 7.69 (d, J=8.0 Hz, 1H), 7.57 (d, J=1.4 Hz, 1H), 7.16 (d, J=1.3 Hz, 1H), 6.88 (d, J=1.4 Hz, 1H), 6.68 (d, J=1.5 Hz, 1H), 6.57 (s, 1H), 3.88 (d, J=11.5 Hz, 2H), 3.66 (s, 2H), 3.20 (s, 3H), 3.09 (s, 3H), 2.60 (dd, J=29.3, 14.4 Hz, 2H), 2.34 (s, 1H), 2.19 (t, J=10.5 Hz, 1H), 2.09-1.90 (m, 2H), 1.45 (d, J=6.7 Hz, 3H), 1.13 (dd, J=8.4, 6.2 Hz, 3H), 0.99 (dt, J=8.2, 3.3 Hz, 2H), 0.94-0.82 (m, 2H). ESI MS [M+H]⁺ for C₃₄H₃₆N₇O₄S, calcd 638.3, found 638.1.

### Example 103: 4-cyclopropyl-6-[6-cyclopropyl-4-[4-fluoro-2-[5-(trifluoromethyl)pyrazol-1-yl]phenyl]pyridin-2-yl]-2-[[(1-methylcyclobutyl)amino]methyl]-1H-pyrrolo[2,3-c]pyridin-7-one

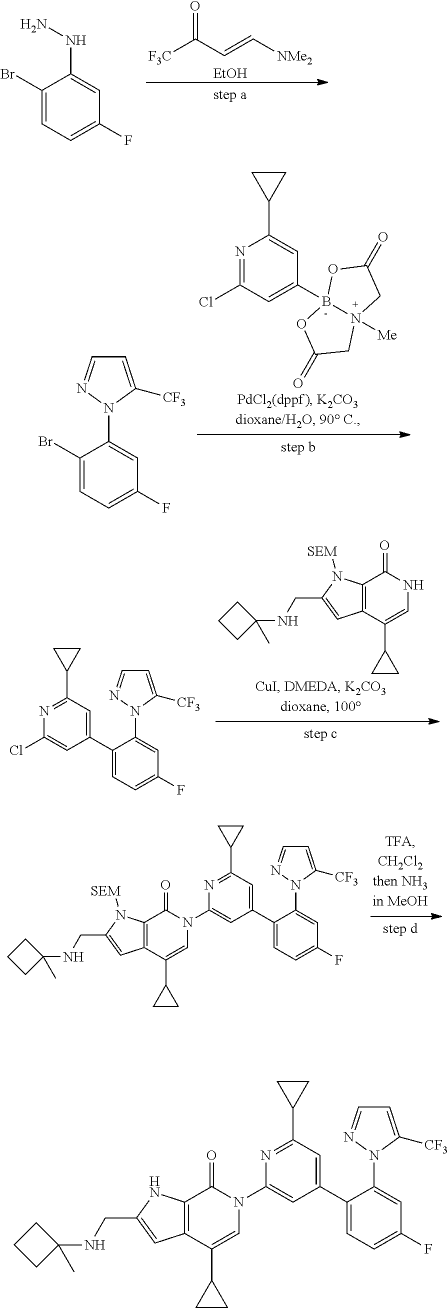

Step a: (E)-4-(dimethylamino)-1,1,1-trifluorobut-3-en-2-one (4.40 mL, 30.5 mmol, 1.20 equiv.) was added to a stirred solution of (2-bromo-5-fluorophenyl)hydrazinehydrochloride (5.0 g, 25.4 mmol, 1.0 equiv.) in ethanol (41 mL, 0.62 M). The reaction was then heated to 60° C. for 16 h under N₂ atmosphere. Reaction was cooled to room temperature and solvent was removed in vacuo. The residue was quenched with sat. aq. NaHCO₃ (10 mL) and extracted with EtOAc (2×20 mL). The combined organics were dried over Na₂SO₄, filtered, and concentrated under vacuum. The crude residue was purified via silica gel flash column chromatography (0 to 40% dichloromethane/hexanes) to afford the desired product.

Step b: A solution of the 1-(2-chloro-6-cyclopropylpyridin-4-yl)-5-methyl-2,8-dioxa-5-azonia-1-boranuidabicyclo[3.3.0]octane-3,7-dione (3.40 g, 10.9 mmol, 1.30 equiv., prepared according to example 18, the product from step a (2.60 g, 8.38 mmol, 1.0 equiv.), and K₂CO₃ (3.50 g, 25.1 mmol, 3.0 equiv.) in dioxane/water (v/v 4:1, 50 mL) was purged with N₂ for 10 minutes. Pd(dppf)Cl₂ (610 mg, 0.84 mmol, 0.1 equiv.) was added, and the reaction was heated to 90° C. and stirred for 16 hours. The reaction was cooled to room temperature, quenched with a ˜1:1 mixture of saturated aqueous NaCl/water (10 mL) and extracted with EtOAc (2×25 mL). The combined organics were dried over Na₂SO₄, filtered, and concentrated under vacuum. The crude residue was then purified by reversed-phase column chromatography (SiO₂-C18, 10-100% CH₃CN in water with 0.1% formic acid) to afford the coupling product.

Step c: A solution of 4-cyclopropyl-2-[[(1-methylcyclobutyl)amino]methyl]-1-(2-trimethylsilylethoxymethyl)-6H-pyrrolo[2,3-c]pyridin-7-one (80 mg, 0.20 mmol, 1.0 equiv., prepared according to example 37), product from step b (100 mg, 0.26 mmol, 1.3 equiv.) and K₂CO₃ (83 mg, 0.60 mmol, 3.0 equiv.) in dioxane (4.0 ml, 0.05 M) was degassed with a stream of nitrogen for ten minutes. CuI (38 mg, 0.20 mmol, 1.0 equiv.) and DMEDA (40 μL, 0.40 mmol, 2.0 equiv.) were added, and the reaction was heated for 16 h at 100° C. Then the mixture was cooled to room temperature, diluted with aq. NH₄Cl (3 mL) and extracted with EtOAc (2×5 mL). The combined organic phase was washed with water (2×5 mL) and brine (5 mL), dried over Na₂SO₄, and concentrated to dryness. The crude residue was purified by reversed-phase column chromatography (SiO₂ C18, 10-100% CH₃CN in water with 0.1% formic acid) to afford desired coupling product.

Step d: The product from the step c (54 mg, 0.072 mmol, 1.0 equiv.) was treated with trifluoroacetic acid/dichloromethane (1 mL, 1:1 v/v) at room temperature for 3 h. The mixture was concentrated under vacuum, then treated with 7M NH₃ in methanol (1 mL) for 30 min followed by the concentration. The crude residue was then purified by reversed-phase column chromatography (SiO₂ C18, 10-100% CH₃CN in water with 0.1% formic acid) to afford the title compound. ¹H NMR (400 MHz, CDCl₃) δ 7.76-7.62 (m, 2H), 7.57 (d, J=1.4 Hz, 1H), 7.35 (td, J=8.2, 2.6 Hz, 1H), 7.24-7.16 (m, 2H), 6.74 (d, J=1.9 Hz, 1H), 6.56 (d, J=1.4 Hz, 1H), 6.32 (s, 1H), 3.88 (s, 2H), 1.93 (td, J=8.2, 4.1 Hz, 3H), 1.86 (p, J=4.8 Hz, 3H), 1.75 (p, J=8.3 Hz, 3H), 1.30 (s, 3H), 1.01-0.80 (m, 6H), 0.70-0.54 (m, 2H). ESI MS [M+H]⁺ for C₃₄H₃₂F₄N₆O calcd 617.3, found 617.3.

### Example 104: 3-[6-cyclopropyl-4-[4-fluoro-2-[5-(trifluoromethyl)pyrazol-1-yl]phenyl]pyridin-2-yl]-6-[(2-methoxyethylamino)methyl]-5H-pyrrolo[3,2-d]pyrimidin-4-one

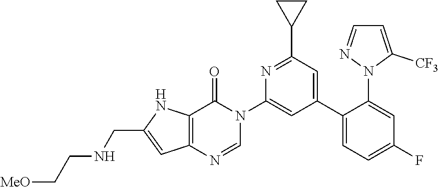 [SMILES: COCCNCc1cc2ncn(-c3cc(-c4ccc(F)cc4-n4nccc4C(F)(F)F)cc(C4CC4)n3)c(=O)c2[nH]1]

The title compound was prepared in a similar fashion to that described for example 28 using 2-chloro-6-cyclopropyl-4-[4-fluoro-2-[5-(trifluoromethyl)pyrazol-1-yl]phenyl]pyridine (prepared according to example 103). ¹H NMR (400 MHz, Chloroform-d) δ 8.23 (s, 1H), 7.67-7.60 (m, 2H), 7.41-7.33 (m, 2H), 7.24 (dd, J=8.3, 2.6 Hz, 1H), 6.77 (d, J=1.4 Hz, 1H), 6.74 (dd, J=2.0, 0.8 Hz, 1H), 6.37 (s, 1H), 4.03 (s, 2H), 3.58-3.47 (m, 2H), 3.37 (s, 3H), 2.92-2.79 (m, 2H), 1.98-1.83 (m, 1H), 1.01-0.85 (m, 4H). ESI MS [M+H]+ for C₂₈H₂₅F₄N₇O₂, calcd 568.2, found 568.1.

### Example 105: 3-[6-cyclopropyl-4-[4-fluoro-2-[5-(trifluoromethyl)pyrazol-1-yl]phenyl]pyridin-2-yl]-6-[[(1-methylcyclobutyl)amino]methyl]-5H-pyrrolo[3,2-d]pyrimidin-4-one

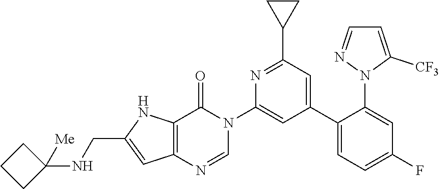 [SMILES: CC1(NCc2cc3ncn(-c4cc(-c5ccc(F)cc5-n5nccc5C(F)(F)F)cc(C5CC5)n4)c(=O)c3[nH]2)CCC1]

The title compound was prepared in a similar fashion to that described for example 28 using 6-[[(1-methylcyclobutyl)amino]methyl]-5-(2-trimethylsilylethoxymethyl)-3H-pyrrolo[3,2-d]pyrimidin-4-one and 2-chloro-6-cyclopropyl-4-[4-fluoro-2-[5-(trifluoro-methyl)pyrazol-1-yl]phenyl]pyridine (prepared according to example 103). ¹H NMR (400 MHz, CDCl₃) δ 8.21 (s, 1H), 7.66 (d, J=1.1 Hz, 1H), 7.61 (dd, J=8.7, 5.8 Hz, 1H), 7.39 (d, J=1.4 Hz, 1H), 7.39-7.34 (m, 1H), 7.29-7.19 (m, 1H), 6.75 (t, J=1.8 Hz, 2H), 6.31 (s, 1H), 3.92 (s, 2H), 2.24-2.06 (m, 2H), 2.00-1.90 (m, 1H), 1.90-1.71 (m, 3H), 1.35 (s, 3H), 1.01-0.84 (m, 4H). ESI MS [M+H]⁺ for C₃₀H₂₇F₄N₇O, calcd 578.2, found 578.3.

### Example 106: 3-[6-cyclopropyl-4-[4-fluoro-2-(4-methylpyridazin-3-yl)phenyl]pyridin-2-yl]-6-[[(2R)-2-methylmorpholin-4-yl]methyl]-5H-pyrrolo[3,2-d]pyrimidin-4-one

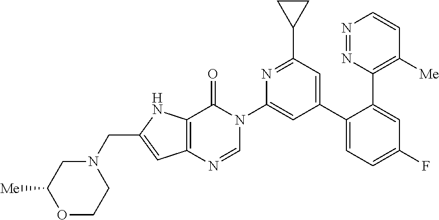 [SMILES: Cc1ccnnc1-c1cc(F)ccc1-c1cc(C2CC2)nc(-n2cnc3cc(CN4CCO[C@H](C)C4)[nH]c3c2=O)c1]

The title compound was prepared in a similar fashion to that described for example 28 using 6-[[(2R)-2-methylmorpholin-4-yl]methyl]-5-(2-trimethylsilylethoxymethyl)-3H-pyrrolo[3,2-d]pyrimidin-4-one and 3-[2-(2-chloro-6-cyclopropylpyridin-4-yl)-5-fluoro-phenyl]-4-methylpyridazine (prepared according to example 75). ¹H NMR (400 MHz, CDCl₃) δ 9.07 (d, J=5.2 Hz, 1H), 8.27 (s, 1H), 7.54 (dd, J=8.7, 5.6 Hz, 1H), 7.39 (d, J=1.3 Hz, 1H), 7.33-7.21 (m, 3H), 6.79 (d, J=1.4 Hz, 1H), 6.47 (s, 1H), 4.38-2.78 (m, 7H), 1.98-1.91 (m, 3H), 1.86 (ddd, J=12.7, 8.1, 4.8 Hz, 1H), 1.18 (d, J=6.3 Hz, 3H), 0.95-0.78 (m, 4H). ESI MS [M+H]⁺ for C₃₁H₃₀FN₇O₂, calcd 552.3, found 552.2.

### Example 107: 2-[2-[2-cyclopropyl-6-[6-[(2-methoxyethylamino)methyl]-4-oxo-5H-pyrrolo[3,2-d]pyrimidin-3-yl]pyridin-4-yl]-5-fluorophenyl]-1-methylimidazole-4-carbonitrile

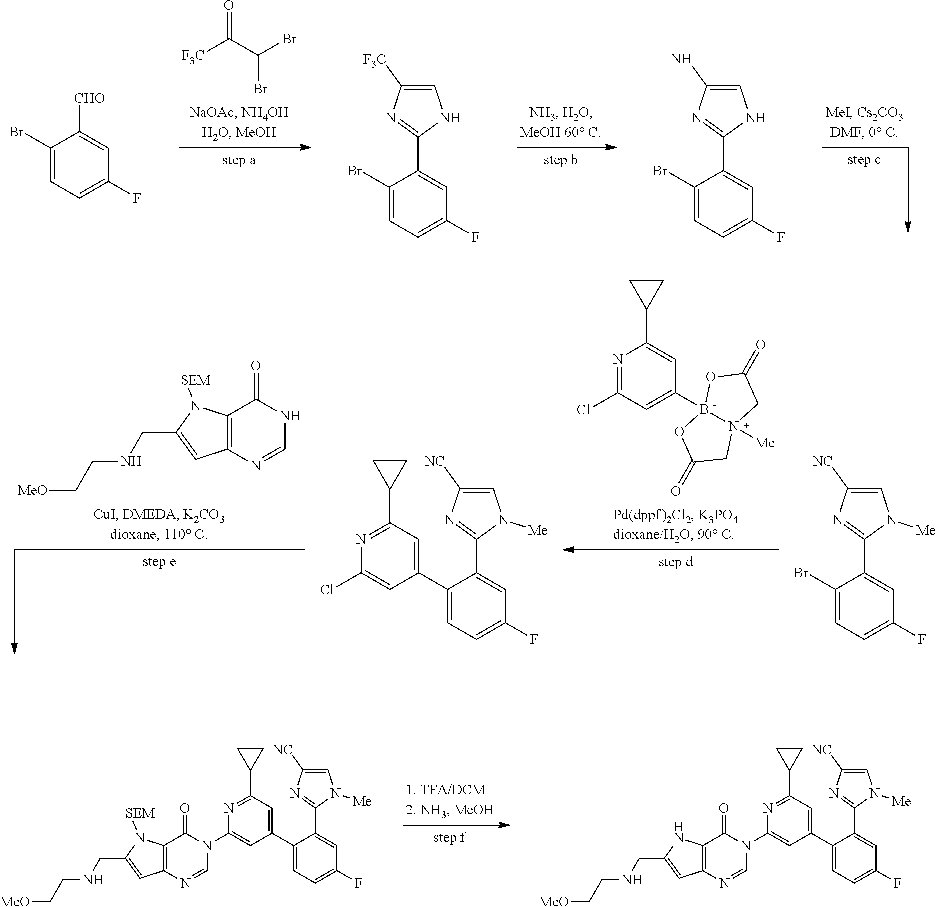

Step a: A suspension of 3,3-dibromo-1,1,1-trifluoropropan-2-one (7.3 g, 27.1 mmol, 1.1 equiv.) and NaOAc (2.2 g, 27.1 mmol, 1.1 equiv.) in water (15 mL) was heated to 100° C. for 45 minutes at which point it was cooled to room temperature. A separate flask was charged with 2-bromo-5-fluorobenzaldehyde (5.0 g, 24.6 mmol, 1.0 equiv.), MeOH (150 mL), and aqueous ammonium hydroxide (28-30% wt, 35 mL). The solution of 3,3-dibromo-1,1,1-trifluoropropan-2-one and NaOAc was then added to the reaction mixture which was stirred for 3 hours at room temperature. The reaction mixture was directly concentrated under reduced pressure and the remaining volume was partitioned between water (300 mL) and EtOAc (100 mL). The aqueous phase was extracted with EtOAc (2×100 mL), and the combined organics were dried over Na₂SO₄, filtered, and concentrated under vacuum. The crude product was used directly without further purification.

Step b: A pressure vessel was charged with the product from step a (7.6 g, 24.7 mmol, 1.0 equiv.), aqueous ammonium hydroxide (28-30% wt, 100 mL), and methanol (10 mL). The pressure vessel was sealed, and the reaction mixture was heated to 60° C. for 24 hours. The reaction mixture was cooled to room temperature and directly concentrated under vacuum to ˜50 mL volume, at which point the product precipitated from solution and was collected via vacuum filtration with water washes and drying on frit until constant weight.

Step c: To a solution of the product from step b (4.4 g, 16.5 mmol, 1.0 equiv.) in DMF (30 mL) was added Cs₂CO₃ (10.8 g, 33.0 mmol, 2.0 equiv.). The reaction mixture was cooled to 0° C. and MeI (1.62 mL, 24.8 mmol, 1.5 equiv.) was added dropwise. The reaction was stirred for 1 hour at 0° C. at which point it was quenched into water (500 mL) and extracted with EtOAc (2×150 mL). The combined organics were dried over Na₂SO₄, filtered, and concentrated under vacuum. The crude residue was purified by column chromatography (SiO₂, EtOAc in hexanes, 20 to 40%) to afford the desired product.

Step d: A round-bottomed flask was charged with the product from step c (2.35 g, 8.4 mmol, 1.0 equiv.), 1-(2-chloro-6-cyclopropylpyridin-4-yl)-5-methyl-2,8-dioxa-5lambda5-aza-1-boranuidabicyclo[3.3.0]octane-3,7-dione (prepared according to example 28), 2.59 g, 8.4 mmol, 1.0 equiv.), K₃PO₄ (5.35 g, 25.2 mmol, 3.0 equiv.), and a 4:1 mixture of dioxane/water (57 mL). The resulting suspension was sparged with N₂ for 10 minutes. Pd(dppf)Cl₂ (615 mg, 0.84 mmol, 0.1 equiv.) was added and the reaction mixture was heated to 90° C. and stirred for 1 hour. The reaction was quenched with a 1:1 mixture of water and brine (300 mL) and extracted with EtOAc (2×150 mL). The combined organics were dried over Na₂SO₄, filtered, and concentrated under vacuum. The crude residue was purified by column chromatography (SiO₂, EtOAc in hexanes, 20 to 100%) to afford the desired product.

Step e: To a suspension of 6-[(2-methoxyethylamino)methyl]-5-(2-trimethylsilylethoxymethyl)-3H-pyrrolo[3,2-d]pyrimidin-4-one (prepared according to Example 28, 70 mg, 0.20 mmol, 1.0 equiv.), the product from step d (72 mg, 0.20 mmol, 1.0 equiv.), and K₂CO₃ (83 mg, 0.60 mmol, 3.0 equiv.) in dioxane (4 mL) was added CuI (38 mg, 0.20 mmol, 1.0 equiv.) and DMEDA (43 uL, 0.40 mmol, 2.0 equiv.). The reaction mixture was heated to 110° C. in a sealed vial and stirred for 16 hours at which point it was quenched with a 1:1 mixture of water/brine (20 mL) and extracted with EtOAc (2×10 mL). The combined organics were dried over Na₂SO₄, filtered, and concentrated under vacuum. The crude residue was purified by column chromatography (SiO₂, MeOH in dichloromethane, 0 to 20%) to afford the desired product.

Step f: To a solution of the product from step e (17 mg, 0.03 mmol, 1.0 equiv.) in dichloromethane (1 mL) was added trifluoroacetic acid (1 mL). The reaction mixture was heated to 30° C. and stirred for 1 hour at which point it was diluted with toluene (5 mL) and concentrated under vacuum. The crude residue was dissolved in 7M NH₃ in MeOH (2 mL) and the resulting reaction mixture was stirred at 30° C. for 1 hour. The reaction was directly concentrated and purified by reverse phase prep-HPLC (SiO₂ C18 column, 5 to 50% MeCN gradient in water with 0.1% trifluoroacetic acid) to afford the desired product. ¹H NMR (400 MHz, CDCl₃) δ 8.31 (s, 1H), 7.56 (dd, J=9.4, 5.3 Hz, 1H), 7.45 (d, J=1.4 Hz, 1H), 7.41 (s, 1H), 7.39-7.30 (m, 2H), 6.81 (d, J=1.4 Hz, 1H), 6.38 (s, 1H), 4.02 (d, J=0.9 Hz, 2H), 3.56-3.49 (m, 2H), 3.39 (s, 3H), 3.17 (s, 3H), 2.88-2.81 (m, 2H), 1.95 (tq, J=9.9, 5.0 Hz, 1H), 1.07-0.93 (m, 3H). ESI MS [M+H]⁺ for C₂₉H₂₇FN₈O₂, calcd 539.2, found 539.3.

### Example 108: 2-{2-[6-cyclopropyl-2-(2-{[(2-methoxyethyl)-N-methylamino]methyl}-7-oxo-1,6-dihydro-1,4,6-triaza-6-indenyl)-4-pyridyl]-5-fluorophenyl}-1-methyl-4-imidazolecarbonitrile

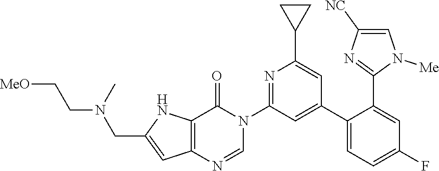 [SMILES: COCCN(C)Cc1cc2ncn(-c3cc(-c4ccc(F)cc4-c4nc(C#N)cn4C)cc(C4CC4)n3)c(=O)c2[nH]1]

The title compound was prepared in a similar fashion to example 107 using 6-[[2-methoxyethyl(methyl)amino]methyl]-3,5-dihydropyrrolo[3,2-d]pyrimidin-4-one (prepared as described in example 22 using 2-methoxy-N-methylethanamine in step d) and 2-[2-(2-chloro-6-cyclopropylpyridin-4-yl)-5-fluorophenyl]-1-methylimidazole-4-carbonitrile (prepared according to example 107). ¹H NMR (400 MHz, CDCl₃) δ 10.37 (s, 1H), 8.31 (s, 1H), 7.59-7.53 (m, 1H), 7.46 (d, J=1.4 Hz, 1H), 7.41 (s, 1H), 7.38-7.30 (m, 2H), 6.80 (d, J=1.4 Hz, 1H), 6.36 (zs, 1H), 3.83 (s, 2H), 3.57 (t, J=5.1 Hz, 2H), 3.42 (s, 3H), 3.17 (s, 3H), 2.69 (t, J=5.1 Hz, 2H), 2.37 (s, 3H), 2.03-1.90 (m, 1H), 1.05-0.99 (m, 2H), 0.97-0.90 (m, 2H); ESI MS [M+H]⁺ C₃₀H₂₉FN₈O₂, calcd 553.2, found 553.2.

### Example 109: 2-[2-(6-cyclopropyl-2-{2-[(3-methoxy-1-azetidinyl)methyl]-7-oxo-1,6-dihydro-1,4,6-triaza-6-indenyl}-4-pyridyl)-5-fluorophenyl]-1-methyl-4-imidazolecarbonitrile

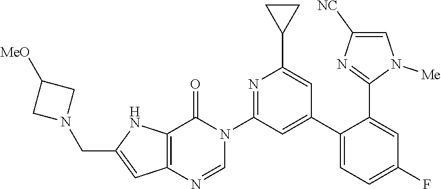 [SMILES: COC1CN(Cc2cc3ncn(-c4cc(-c5ccc(F)cc5-c5nc(C#N)cn5C)cc(C5CC5)n4)c(=O)c3[nH]2)C1]

The title compound was prepared in a similar fashion to example 107 using 6-[[2-methoxyethyl(methyl)amino]methyl]-3,5-dihydropyrrolo[3,2-d]pyrimidin-4-one (prepared as described in example 22 using 3-methoxyazetidine in step d) and 2-[2-(2-chloro-6-cyclopropylpyridin-4-yl)-5-fluorophenyl]-1-methylimidazole-4-carbonitrile (prepared according to example 107). ¹H NMR (400 MHz, CDCl₃) δ 9.75 (s, 1H), 8.31 (s, 1H), 7.59-7.52 (m, 1H), 7.46-7.38 (m, 2H), 7.37-7.31 (m, 2H), 6.82 (d, J=1.3 Hz, 1H), 6.38 (s, 1H), 4.11-4.02 (m, 1H), 3.81 (s, 2H), 3.69-3.61 (m, 2H), 3.27 (s, 3H), 3.17 (s, 3H), 3.10-3.06 (m, 2H), 2.00-1.91 (m, 1H), 1.06-0.99 (m, 2H), 0.98-0.91 (m, 2H); ESI MS [M+H]⁺ C₃₀H₂₇FN₈O₂, calcd 551.2, found 551.2.

### Example 110: 2-[2-[2-cyclopropyl-6-[4-cyclopropyl-2-[(2-methoxyethylamino)methyl]-7-oxo-1H-pyrrolo[2,3-c]pyridin-6-yl]pyridin-4-yl]-5-fluorophenyl]-1-methylimidazole-4-carbonitrile

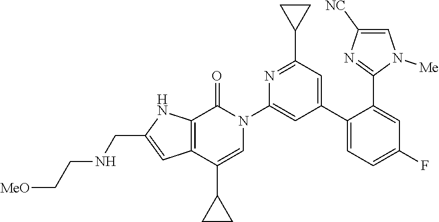 [SMILES: COCCNCc1cc2c(C3CC3)cn(-c3cc(-c4ccc(F)cc4-c4nc(C#N)cn4C)cc(C4CC4)n3)c(=O)c2[nH]1]

The title compound was prepared in a similar fashion to that described for example 107 using 4-cyclopropyl-2-[(2-methoxyethylamino)methyl]-1-(2-trimethylsilylethoxy-methyl)-6H-pyrrolo[2,3-c]pyridin-7-one (prepared in an analogous fashion to example 1 using 2-methoxyethyl amine on step e) and 2-[2-(2-chloro-6-cyclopropylpyridin-4-yl)-5-fluorophenyl]-1-methylimidazole-4-carbonitrile (prepared according to example 107). ¹H NMR (400 MHz, CDCl₃) δ 7.61 (d, J=1.5 Hz, 1H), 7.58-7.49 (m, 1H), 7.39 (s, 1H), 7.36-7.28 (m, 2H), 7.24 (d, J=1.3 Hz, 1H), 6.60 (d, J=1.4 Hz, 1H), 6.38 (s, 1H), 4.00 (s, 2H), 3.52 (t, J=4.9 Hz, 2H), 3.37 (s, 3H), 3.17 (s, 3H), 2.83 (t, J=4.9 Hz, 2H), 2.04-1.82 (m, 2H), 1.07-0.96 (m, 2H), 0.93-0.84 (m, 4H), 0.76-0.58 (m, 2H). ESI MS [M+H]⁺ for C₃₃H₃₂FN₇O₂, calcd 578.3, found 578.4.

### Example 111: 2-[2-[2-cyclopropyl-6-[6-[[[(2S)-1-methoxypropan-2-yl]amino]methyl]-4-oxo-5H-pyrrolo[3,2-d]pyrimidin-3-yl]pyridin-4-yl]-5-fluorophenyl]-1-methylimidazole-4-carbonitrile

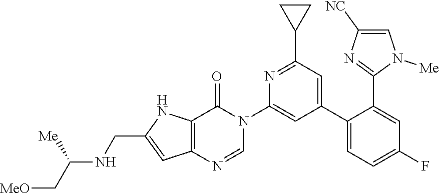 [SMILES: COC[C@H](C)NCc1cc2ncn(-c3cc(-c4ccc(F)cc4-c4nc(C#N)cn4C)cc(C4CC4)n3)c(=O)c2[nH]1]

The title compound was prepared in a similar fashion to that described for example 107 using 6-[[[(2S)-1-methoxypropan-2-yl]amino]methyl]-5-(2-trimethylsilylethoxymethyl)-3H-pyrrolo[3,2-d]pyrimidin-4-one (prepared in an analogous manner to that described in example 22) and 2-[2-(2-chloro-6-cyclopropylpyridin-4-yl)-5-fluorophenyl]-1-methyl-imidazole-4-carbonitrile. ¹H NMR (400 MHz, CDCl₃) δ 8.29 (s, 1H), 7.56 (dd, J=9.4, 5.4 Hz, 1H), 7.45 (d, J=1.5 Hz, 1H), 7.40 (s, 1H), 7.34 (ddd, J=7.9, 4.3, 1.9 Hz, 2H), 6.79 (d, J=1.5 Hz, 1H), 6.34 (d, J=0.9 Hz, 1H), 4.00 (dd, J=3.7, 0.8 Hz, 2H), 3.42-3.33 (m, 4H), 3.25 (dd, J=9.4, 7.7 Hz, 1H), 3.17 (s, 3H), 3.00-2.88 (m, 1H), 1.99-1.90 (m, 1H), 1.03 (d, J=6.5 Hz, 3H), 1.05-0.90 (m, 4H). ESI MS [M+H]⁺ for C₃₀H₂₉FN₈O₂, calcd 553.2, found 553.3.

### Example 112: 2-[2-[2-cyclopropyl-6-[4-oxo-6-[(3,3,3-trifluoropropylamino)methyl]-5H-pyrrolo[3,2-d]pyrimidin-3-yl]pyridin-4-yl]-5-fluorophenyl]-1-methylimidazole-4-carbonitrile

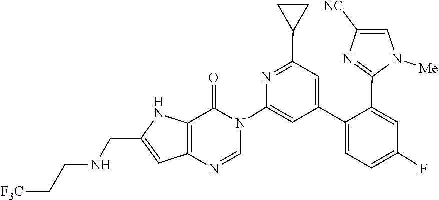 [SMILES: Cn1cc(C#N)nc1-c1cc(F)ccc1-c1cc(C2CC2)nc(-n2cnc3cc(CNCCC(F)(F)F)[nH]c3c2=O)c1]

The title compound was prepared in a similar fashion to that described for Example 107 using 6-[(3,3,3-trifluoropropylamino)methyl]-5-(2-trimethylsilylethoxymethyl)-3H-pyrrolo[3,2-d]pyrimidin-4-one (prepared in an analogous manner to that described in example 22) and 2-[2-(2-chloro-6-cyclopropylpyridin-4-yl)-5-fluorophenyl]-1-methylimidazole-4-carbonitrile. ¹H NMR (400 MHz, CDCl₃) δ 9.92 (s, 1H), 8.30 (s, 1H), 7.56 (dd, J=9.4, 5.4 Hz, 1H), 7.42 (d, J=1.4 Hz, 1H), 7.41 (s, 1H), 7.38-7.31 (m, 2H), 6.84 (d, J=1.4 Hz, 1H), 6.39 (s, 1H), 3.97 (s, 2H), 3.17 (s, 3H), 2.89 (t, J=7.0 Hz, 2H), 2.38-2.24 (m, 2H), 2.00 (s, 2H), 1.99-1.92 (m, 1H), 1.06-0.99 (m, 2H), 0.98-0.92 (m, 2H). ESI MS [M+H]⁺ for C₂₉H₂₄F₄N₈O, calcd 577.2, found 577.2.

### Example 113: 2-[2-[2-cyclopropyl-6-[6-[[[(2S)-2-methoxypropyl]-methylamino]methyl]-4-oxo-5H-pyrrolo[3,2-d]pyrimidin-3-yl]pyridin-4-yl]-5-fluorophenyl]-1-methyl-imidazole-4-carbonitrile

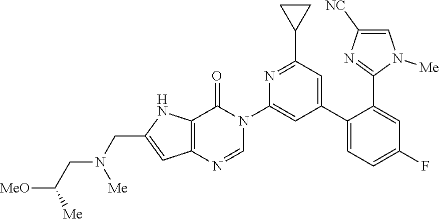 [SMILES: CO[C@@H](C)CN(C)Cc1cc2ncn(-c3cc(-c4ccc(F)cc4-c4nc(C#N)cn4C)cc(C4CC4)n3)c(=O)c2[nH]1]

The title compound was prepared in a similar fashion to that described for Example 107 using 6-[[[(2S)-2-methoxypropyl]-methylamino]methyl]-5-(2-trimethylsilylethoxy-methyl)-3H-pyrrolo[3,2-d]pyrimidin-4-one (prepared in an analogous manner to that described in example 22) and 2-[2-(2-chloro-6-cyclopropylpyridin-4-yl)-5-fluorophenyl]-1-methyl-imidazole-4-carbonitrile. ¹H NMR (400 MHz, CDCl₃) δ 8.30 (s, 1H), 7.56 (dd, J=9.4, 5.4 Hz, 1H), 7.46 (d, J=1.4 Hz, 1H), 7.41 (s, 1H), 7.37-7.31 (m, 2H), 6.79 (d, J=1.4 Hz, 1H), 6.33 (s, 1H), 3.90-3.74 (m, 2H), 3.65-3.52 (m, 1H), 3.44 (s, 3H), 3.17 (s, 3H), 2.59 (dd, J=13.4, 8.2 Hz, 1H), 2.44-2.39 (m, 1H), 2.37 (s, 3H), 1.99-1.90 (m, 1H), 1.14 (d, J=6.2 Hz, 3H), 1.07-0.98 (m, 2H), 0.98-0.90 (m, 2H). ESI MS [M+H]⁺ for C₃₁H₃₁FN₈O₂, calcd 567.3, found 567.3.

### Example 114: 2-[5-cyano-2-(6-cyclopropyl-2-{4-cyclopropyl-2-[(1-methylcyclobutylamino)methyl]-7-oxo-1,6-dihydro-1,6-diaza-6-indenyl}-4-pyridyl)phenyl]-1-methyl-4-imidazolecarbonitrile

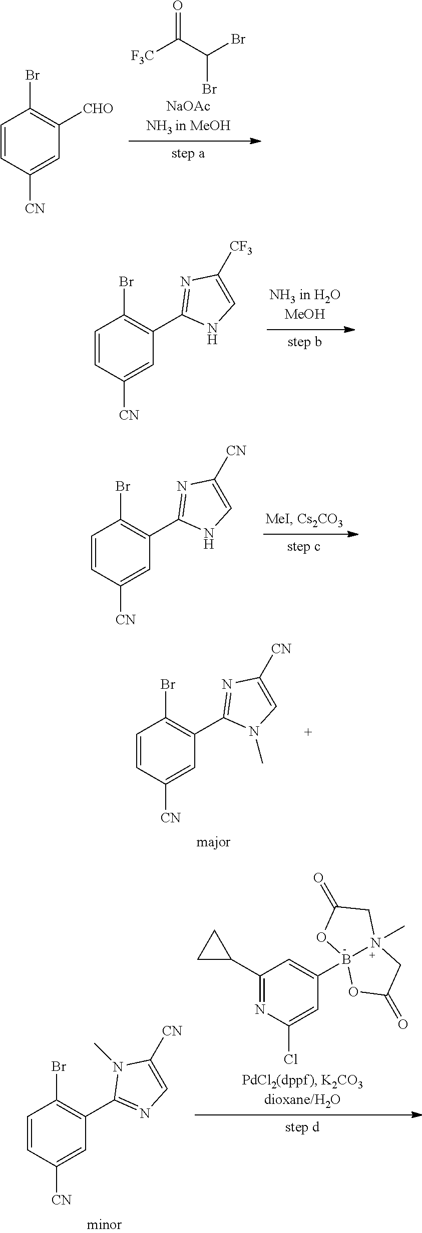

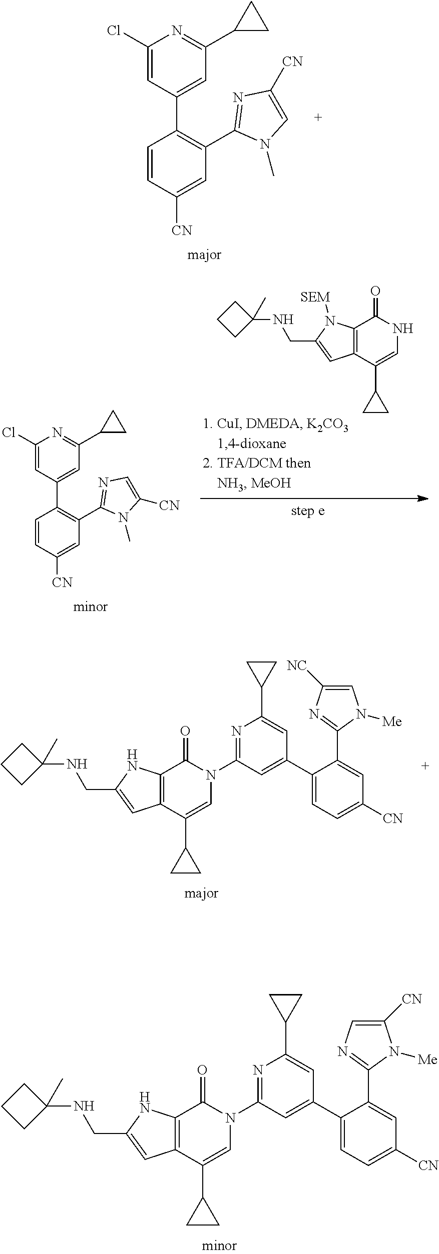 [SMILES: Cn1cc(C#N)nc1-c1cc(C#N)ccc1-c1cc(Cl)nc(C2CC2)c1 ; Cn1c(C#N)cnc1-c1cc(C#N)ccc1-c1cc(Cl)nc(C2CC2)c1 ; CC1(NCc2cc3c(C4CC4)c[nH]c(=O)c3n2COCC[Si](C)(C)C)CCC1 ; Cn1cc(C#N)nc1-c1cc(C#N)ccc1-c1cc(C2CC2)nc(-n2cc(C3CC3)c3cc(CNC4(C)CCC4)[nH]c3c2=O)c1 ; Cn1c(C#N)cnc1-c1cc(C#N)ccc1-c1cc(C2CC2)nc(-n2cc(C3CC3)c3cc(CNC4(C)CCC4)[nH]c3c2=O)c1]

Step a: A mixture of 3,3-dibromo-1,1,1-trifluoropropan-2-one (4.45 g, 16.5 mmol, 1.1 equiv.) and sodium acetate (1.35 g, 16.5 mmol, 1.1 equiv.) in water (10 mL) was heated under stirring at 100° C. for 1 h. The resulting mixture was cooled to 0° C. To this solution a mixture of 4-bromo-3-formylbenzonitrile (3.15 g, 15 mmol, 1.0 equiv.) and concentrated aqueous ammonium hydroxide (20 ml) was added, and the resulting mixture was stirred at room temperature for 4 h. All volatiles were removed under vacuum, and the residue was partitioned between EtOAc (30 mL) and water (30 mL). The organic phase was separated, and the aqueous layer was extracted with EtOAc (20 mL). The combined organic extract was dried over Na₂SO₄ and concentrated to dryness under reduced pressure. The crude condensation product was purified by column chromatography (SiO₂, EtOAc in hexanes, 0 to 20%) to afford 4-bromo-3-[4-(trifluoromethyl)-1H-imidazol-2-yl]benzonitrile.

Step b: The product from step a (500 mg, 1.5873 mmol, 1.0 equiv.) was suspended in a mixture of concentrated aqueous ammonium hydroxide solution (5 mL) and methanol (5 mL). This solution was placed in a sealed tube and heated at 60° C. overnight. The resulting mixture was allowed to cool to room temperature, diluted with water and extracted with EtOAc (3×15 mL). The combined organic extract was dried over Na₂SO₄ and concentrated to dryness under reduced pressure. The crude residue was purified by column chromatography (SiO₂, EtOAc in hexanes, 0 to 20%) to give 2-(2-bromo-5-cyanophenyl)-1H-imidazole-4-carbonitrile.

Step c: To a solution of the product of step b (200 mg, 0.63 mmol, 1.0 equiv.) and Cs₂CO₃ (413 mg, 1.27 mmol, 3.0 equiv.) in THE (5 mL, 0.12 M) at 0° C. was added MeI (141 mg, 0.95 mmol, 1.5 equiv.). The resulting mixture was stirred at 23° C. for 1 h, then quenched with saturated aqueous NH₄Cl solution (10 mL) and diluted with EtOAc. The organic phase was separated, and the aqueous layer was extracted with EtOAc (2×7 mL). The combined organic phase was dried over Na₂SO₄, the solvent was removed under reduced pressure, and the crude alkylation product was purified by column chromatography (SiO₂, MeOH in dichloromethane, 0 to 10%) to afford 2-(2-bromo-5-cyanophenyl)-1-methylimidazole-4-carbonitrile along with 2-(2-bromo-5-cyanophenyl)-3-methylimidazole-4-carbonitrile as an inseparable mixture in 4:1 ratio respectively.

Step d: To a solution of the product from step c (94 mg, 0.30 mmol, 1.0 equiv., mixture of regioisomers in 4:1 ratio), 1-(2-chloro-6-cyclopropylpyridin-4-yl)-5-methyl-2,8-dioxa-5-azonia-1-boranuidabicyclo[3.3.0]octane-3,7-dione (87 mg, 0.30 mmol, 1.0 equiv., prepared according to example 18) and K₂CO₃ (0.13 g, 0.9093 mmol, 3.0 equiv.) in dioxane/water mixture (3 mL, 2:1 v/v) was added Pd(dppf)Cl₂ (24 m g, 0.03 mmol, 0.1 equiv.). The resulting mixture was degassed by three cycles of applying vacuum and backfilling with nitrogen and stirred at 90° C. for 1 h. The reaction was cooled to room temperature, diluted with sat. aq. NH₄Cl solution (5 mL) and EtOAc (10 mL). The organic phase was separated, and the aqueous phase was additionally extracted with EtOAc (10 mL). The combined organic extract was dried over Na₂SO₄, all volatiles were evaporated under reduced pressure, and the crude coupling product was purified by column chromatography (SiO₂, EtOAc in hexanes, 0 to 60%) to furnish 2-[2-(2-chloro-6-cyclopropylpyridin-4-yl)-5-cyanophenyl]-1-methylimidazole-4-carbonitrile along with its regioisomer.

Step e: To a mixture of the product of step d (60 mg, 0.17 mmol, 1.0 equiv.), 4-(2-chloro-6-cyclopropylpyridin-4-yl)-3-(1-methylimidazol-2-yl)benzonitrile (68 mg, 0.17 mmol, 1.0 equiv., prepared according to example 37 in dioxane (3.0 mL) was added CuI (32 mg, 0.17 mmol, 1.0 equiv.), N,N′-dimethylethylenediamine (58.8 mg, 0.67 mmol, 4.0 equiv.) and K₂CO₃ (69 mg, 0.50 mmol, 3.0 equiv.) The resulting mixture was sparged with nitrogen for 10 min and heated at 110° C. overnight. The resulting suspension was cooled to room temperature and partitioned between EtOAc (10 mL) and aq. sat. NH₄Cl (10 mL). The organic phase was separated, and the aqueous phase was extracted with EtOAc (10 mL). The combined organic extract was dried over Na₂SO₄ and concentrated to dryness under reduced pressure. The crude coupling product was dissolved in trifluoroacetic acid/dichloromethane mixture (2 mL, 1:10 v/v) and stirred at 23° C. for 3 h. Upon solvents evaporation under vacuum the dry residue was redissolved in NH₃ in methanol (7M, 2 mL). After 30 min the solvent was evaporated, and the product was purified by prep-HPLC (SiO₂ C18, 10 to 90% MeCN in water with 0.1% trifluoroacetic acid) to afford the title compound along with minor regioisomer. (N-methylpyrazole regioisomeric ratio 4:1). ¹H NMR (400 MHz, CDCl₃, major regioisomer) δ 8.01-7.82 (m, 2H), 7.74-7.61 (m, 2H), 7.42 (s, 1H), 7.23 (d, J=1.2 Hz, 1H), 6.65 (d, J=1.3 Hz, 1H), 6.35 (s, 1H), 3.93 (s, 2H), 3.18 (s, 3H), 2.11 (d, J=10.0 Hz, 2H), 1.99-1.67 (m, 7H), 1.37 (d, J=6.2 Hz, 3H), 1.03 (dt, J=7.9, 3.2 Hz, 2H), 0.97-0.84 (m, 4H), 0.67 (tt, J=6.2, 3.7 Hz, 2H). ESI MS [M+H]⁺ for C₃₆H₃₅N₈O, calcd 595.3, found 595.1.

### Example 115: 2-[5-cyano-2-[2-cyclopropyl-6-[6-[[(1-methylcyclobutyl)amino]methyl]-4-oxo-5H-pyrrolo[3,2-d]pyrimidin-3-yl]pyridin-4-yl]phenyl]-1-methylimidazole-4-carbonitrile

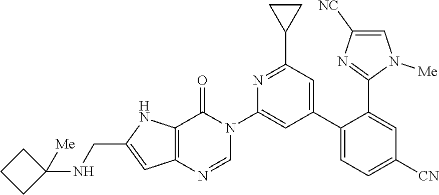 [SMILES: Cn1cc(C#N)nc1-c1cc(C#N)ccc1-c1cc(C2CC2)nc(-n2cnc3cc(CNC4(C)CCC4)[nH]c3c2=O)c1]

The title compound was prepared in a similar fashion to that described for example 28 using 6-[[(1-methylcyclobutyl)amino]methyl]-5-(2-trimethylsilylethoxymethyl)-3H-pyrrolo[3,2-d]pyrimidin-4-one (obtained according to example 28 using 1-methylcyclobutan-1-amine for reductive amination step) and 1-methylcyclobutan-1-amine and 2-[2-(2-chloro-6-cyclopropylpyridin-4-yl)-5-cyanophenyl]-1-methylimidazole-4-carbonitrile (prepared according to example 114). ¹H NMR (400 MHz, CDCl₃) δ 8.28 (s, 1H), 7.93-7.89 (m, 2H), 7.72-7.65 (m, 1H), 7.48-7.44 (m, 2H), 6.85 (d, J=1.4 Hz, 1H), 6.37 (s, 1H), 3.98 (s, 2H), 3.19 (s, 3H), 2.20-2.07 (m, 2H), 2.05-1.95 (m, 1H), 1.92-1.72 (m, 3H), 1.39 (s, 3H), 1.10-1.01 (m, 2H), 1.00-0.91 (m, 2H). ESI MS [M+H]⁺ for C₃₂H₂₉N₉O, calcd 556.3, found 556.3.

### Example 116: 2-[5-cyano-2-[2-cyclopropyl-6-[6-[(2-methoxyethylamino)methyl]-4-oxo-5H-pyrrolo[3,2-d]pyrimidin-3-yl]pyridin-4-yl]phenyl]-1-methylimidazole-4-carbonitrilef

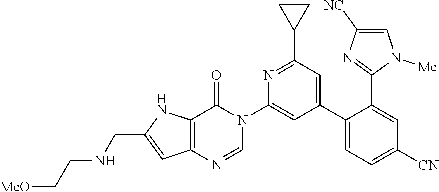 [SMILES: COCCNCc1cc2ncn(-c3cc(-c4ccc(C#N)cc4-c4nc(C#N)cn4C)cc(C4CC4)n3)c(=O)c2[nH]1]

The title compound was prepared in a similar fashion to that described for example 28 using 6-[(2-methoxyethylamino)methyl]-5-(2-trimethylsilylethoxymethyl)-3H-pyrrolo[3,2-d]pyrimidin-4-one (prepared according to example 28) and 2-[2-(2-chloro-6-cyclopropylpyridin-4-yl)-5-cyanophenyl]-1-methylimidazole-4-carbonitrile (prepared according to example 114). ¹H NMR (400 MHz, CDCl₃) δ 8.32 (s, 1H), 7.97-7.88 (m, 2H), 7.70 (dd, J=7.9, 0.8 Hz, 1H), 7.49 (d, J=1.4 Hz, 1H), 7.45 (s, 1H), 6.86 (d, J=1.4 Hz, 1H), 6.37 (s, 1H), 4.01 (s, 2H), 3.52 (t, J=4.7 Hz, 2H), 3.39 (s, 3H), 3.19 (s, 3H), 2.84 (t, J=4.8 Hz, 2H), 2.01-1.93 (m, 1H), 1.10-0.94 (m, 4H). ESI MS [M+H]⁺ for C₃₀H₂₇N₉O₂, calcd 546.2, found 546.3.

### Example 117: 4-[2-(2-{[(S)-3-methyl-1-piperidyl]methyl}-4-cyclopropyl-7-oxo-1,6-dihydro-1,6-diaza-6-indenyl)-6-cyclopropyl-4-pyridyl]-3-[1-methyl-4-(trifluoromethyl)-2-imidazolyl]benzonitrile

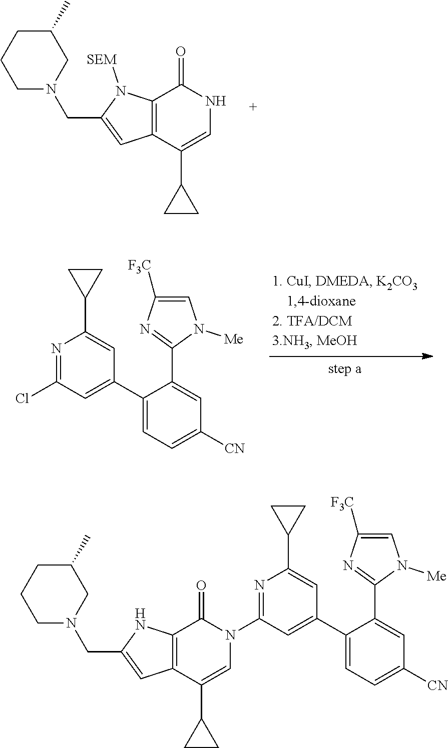 [SMILES: C[C@H]1CCCN(Cc2cc3c(C4CC4)c[nH]c(=O)c3n2COCC[Si](C)(C)C)C1 ; Cn1cc(C(F)(F)F)nc1-c1cc(C#N)ccc1-c1cc(Cl)nc(C2CC2)c1 ; C[C@H]1CCCN(Cc2cc3c(C4CC4)cn(-c4cc(-c5ccc(C#N)cc5-c5nc(C(F)(F)F)cn5C)cc(C5CC5)n4)c(=O)c3[nH]2)C1]

Step a: To a mixture of 4-cyclopropyl-2-[[(3S)-3-methylpiperidin-1-yl]methyl]-1-(2-trimethylsilylethoxymethyl)-6H-pyrrolo[2,3-c]pyridin-7-one (41.5 mg, 0.1 mmol, 1.0 equiv., prepared according to example 1), 4-(2-chloro-6-cyclopropylpyridin-4-yl)-3-[1-methyl-4-(trifluoromethyl)imidazol-2-yl]benzonitrile (40 mg, 0.1 mmol, 1.0 equiv., prepared according to example 67) in dioxane (3.0 mL) was added CuI (19 mg, 0.1 mmol, 1.0 equiv.), N,N′-dimethylethylenediamine (35 mg, 0.4 mmol, 4.0 equiv.) and K₂CO₃ (42 mg, 0.3 mmol, 3.0 equiv.) The resulting mixture was heated at 110° C. overnight. The reaction mixture was cooled to room temperature, diluted with EtOAc, washed with H₂O and brine, filtered through Na₂SO₄, and concentrated. The residual material was then treated with TFA/DCM (v/v 1:10, 2 mL) at room temperature for 3 h. The mixture was concentrated under vacuum, then treated with 7M NH₃ in methanol (2 mL) for 30 min followed by concentration under vacuum. The crude residue was then purified by prep-HPLC to afford title compound (regioisomeric ratio 8:1). ¹H NMR (400 MHz, CDCl₃, major isomer) δ 7.97 (d, J=1.7 Hz, 1H), 7.87 (dd, J=8.1, 1.8 Hz, 1H), 7.78 (d, J=1.4 Hz, 1H), 7.67 (d, J=8.2 Hz, 1H), 7.29 (d, J=1.2 Hz, 1H), 7.19 (d, J=1.4 Hz, 1H), 6.68-6.49 (m, 1H), 6.41 (s, 1H), 3.76 (d, J=3.3 Hz, 2H), 3.14 (s, 3H), 2.92 (dd, J=27.2, 9.0 Hz, 2H), 2.10 (m, 1H), 1.96-1.86 (m, 3H), 1.78 (d, J=5.1 Hz, 3H), 1.69 (d, J=8.4 Hz, 3H), 0.97 (dt, J=8.1, 3.4 Hz, 2H), 0.91-0.79 (m, 6H), 0.68 (td, J=5.9, 4.1 Hz, 2H). ESI MS [M+H]⁺ for C₃₇H₃₇F₃N₇O, calcd 652.3, found 652.1.

### Example 118: 3-[6-cyclopropyl-4-[4-fluoro-2-(2-methyl-1,2,4-triazol-3-yl)phenyl]-pyridin-2-yl]-6-[(2-methoxyethylamino)methyl]-5H-pyrrolo[3,2-d]pyrimidin-4-one

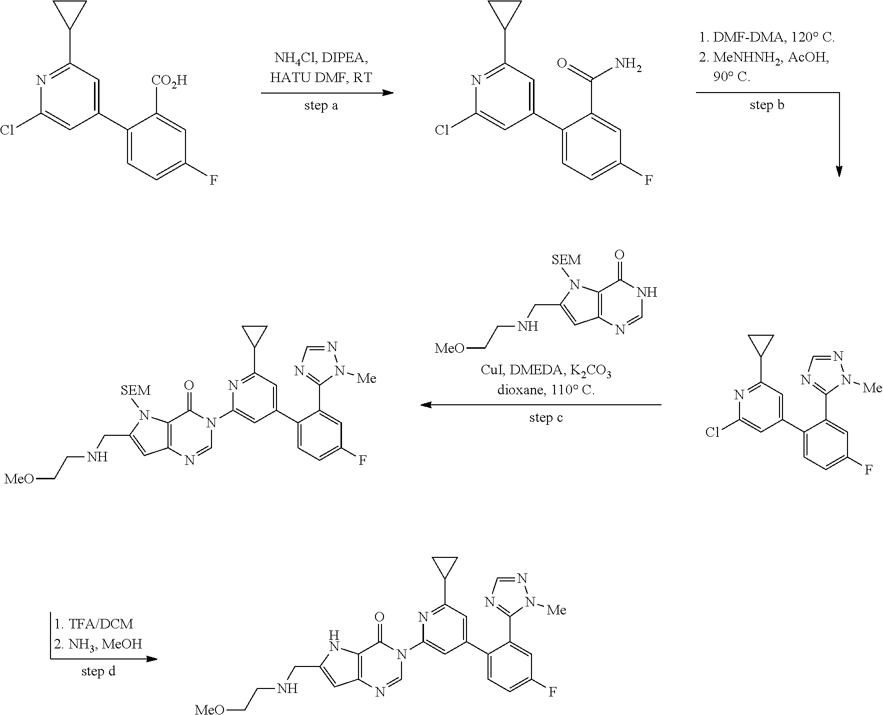 [SMILES: O=C(O)c1cc(F)ccc1-c1cc(Cl)nc(C2CC2)c1 ; NC(=O)c1cc(F)ccc1-c1cc(Cl)nc(C2CC2)c1 ; COCCNCc1cc2ncn(-c3cc(-c4ccc(F)cc4-c4ncnn4C)cc(C4CC4)n3)c(=O)c2n1COCC[Si](C)(C)C ; COCCNCc1cc2nc[nH]c(=O)c2n1COCC[Si](C)(C)C ; Cn1ncnc1-c1cc(F)ccc1-c1cc(Cl)nc(C2CC2)c1 ; COCCNCc1cc2ncn(-c3cc(-c4ccc(F)cc4-c4ncnn4C)cc(C4CC4)n3)c(=O)c2[nH]1]

Step a: To a suspension of 2-(2-chloro-6-cyclopropylpyridin-4-yl)-5-fluorobenzoic acid (5.0 g, 17.1 mmol, 1.0 equiv.) and NH₄Cl (1.8 g, 34.2 mmol, 2.0 equiv.) in DMF (35 mL) was added DIPEA (12 mL, 68.4 mmol, 4.0 equiv.) followed by HATU (6.5 g, 17.1 mmol, 1.0 equiv.). The reaction mixture was stirred at room temperature for 16 hours at which point it was partitioned between water (500 mL) and EtOAc (200 mL). The aqueous phase was extracted with EtOAc (2×100 mL) and the combined organics were dried over Na₂SO₄, filtered, and concentrated under vacuum. The crude residue was purified by column chromatography (SiO₂, EtOAc in hexanes, 0 to 80%) to afford the desired product.

Step b: A pressure vessel was charged with the product from step a (4.4 g, 15.2 mmol, 1.0 equiv.) and DMF-DMA (30 mL). The pressure vessel was sealed, and the reaction mixture was heated to 120° C. for 1 hour. The reaction mixture was cooled to room temperature and directly concentrated. The crude residue was dissolved in acetic acid (30 mL). Methylhydrazine (1.0 mL, 18.2 mmol, 1.2 equiv.) was added and the reaction mixture was heated to 90° C. and stirred for 2.5 hours. The reaction mixture was directly concentrated under vacuum and the crude residue was partitioned between saturated aqueous NaHCO₃ (300 mL) and EtOAc (150 mL). The aqueous phase was extracted with EtOAc (2×100 mL) and the combined organics were dried over Na₂SO₄, filtered, and concentrated under vacuum. The crude residue was purified by column chromatography (SiO₂, EtOAc in hexanes, 0 to 100%) to afford the desired product.

Step c: To a suspension of 6-[(2-methoxyethylamino)methyl]-5-(2-trimethylsilylethoxymethyl)-3H-pyrrolo[3,2-d]pyrimidin-4-one (50 mg, 0.14 mmol, 1.0 equiv., prepared according to example 28), the product from step b (47 mg, 0.14 mmol, 1.0 equiv.), and K₂CO₃ (59 mg, 0.43 mmol, 3.0 equiv.) in dioxane (3 mL) was added CuI (27 mg, 0.14 mmol, 1.0 equiv.) and DMEDA (31 μL, 0.28 mmol, 2.0 equiv.). The reaction mixture was heated to 110° C. and stirred for 16 hours at which point it was quenched with a 1:1 mixture of brine and water (20 mL) and extracted with EtOAc (2×10 mL). The combined organics were dried over Na₂SO₄, filtered, and concentrated under vacuum. The crude residue was purified by column chromatography (SiO₂, MeOH in dichloromethane, 0 to 20%) to afford the desired product.

Step d: To a solution of the product from step c (48 mg, 0.07 mmol, 1.0 equiv.) in dichloromethane (1 mL) was added trifluoroacetic acid (1 mL). The reaction mixture was heated to 30° C. and stirred for 1 hour at which point it was diluted with toluene (5 mL) and concentrated under vacuum. The crude residue was dissolved in 7M NH₃ in MeOH (2 mL) and the resulting reaction mixture was stirred at 30° C. for 1 hour. The reaction was directly concentrated and purified by reverse phase prep-HPLC (SiO₂ C18 column, 5 to 50% MeCN in water with 0.1% trifluoroacetic acid) to afford the desired product. ¹H NMR (400 MHz, CDCl₃) δ 8.33 (s, 1H), 7.96 (s, 1H), 7.60 (dd, J=8.3, 5.3 Hz, 1H), 7.51 (d, J=1.4 Hz, 1H), 7.40-7.30 (m, 2H), 6.74 (d, J=1.5 Hz, 1H), 6.36 (s, 1H), 4.00 (s, 2H), 3.51 (t, J=4.9 Hz, 2H), 3.39 (s, 6H), 2.83 (d, J=4.9 Hz, 2H), 1.96-1.86 (m, 1H), 1.01-0.90 (m, 4H). ESI MS [M+H]⁺ for C₂₇H₂₇FN₈O₂, calcd 515.2, found 515.3.

### Example 119: 4-Cyclopropyl-6-[6-cyclopropyl-4-[4-fluoro-2-(2-methyl-1,2,4-triazol-3-yl)phenyl]pyridin-2-yl]-2-[[(3S)-3-methylpiperidin-1-yl]methyl]-1H-pyrrolo[2,3-c]pyridin-7-one

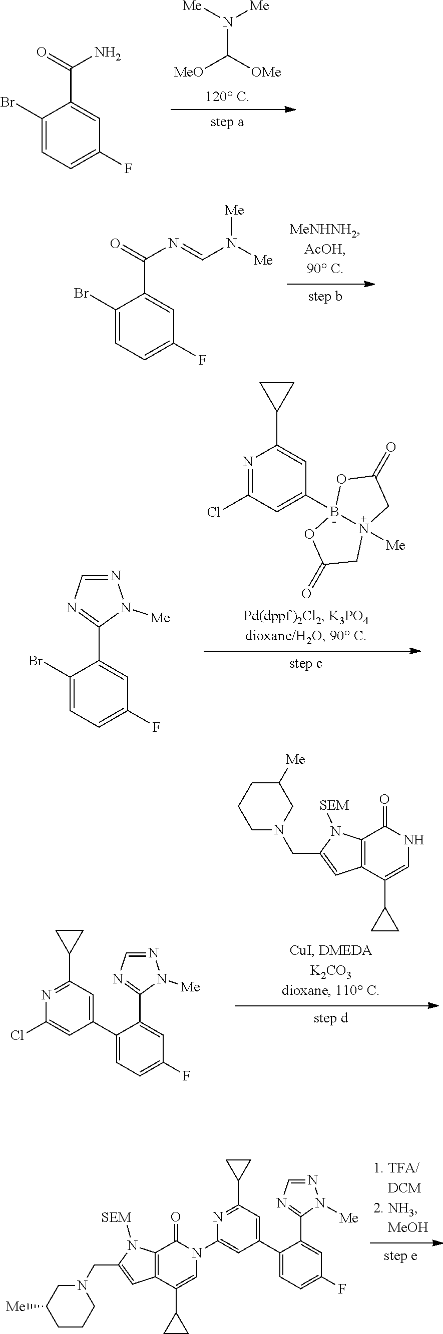

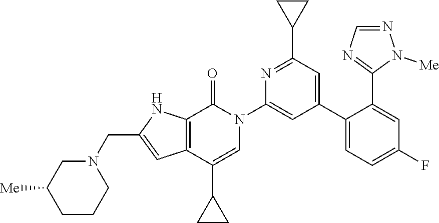 [SMILES: C[C@H]1CCCN(Cc2cc3c(C4CC4)cn(-c4cc(-c5ccc(F)cc5-c5ncnn5C)cc(C5CC5)n4)c(=O)c3[nH]2)C1]

Step a: A solution of 2-bromo-5-fluorobenzamide (1.0 g, 4.59 mmol, 1.0 equiv.) in 1,1-dimethoxy-N,N-dimethylmethanamine (3 mL) was heated at 120° C. under N₂ for 2 h. The reaction mixture was concentrated under reduced pressure and directly used in the next step.

Step b: To a solution of the product from step a (4.59 mmol assumed, 1.0 equiv.) in AcOH (9 mL) was added methylhydrazine (0.29 mL, 5.51 mmol, 1.2 equiv.). The reaction mixture was heated at 90° C. under N₂ for 5 h before cooling to room temperature. The obtained solution was partitioned between water (50 mL) and EtOAc (50 mL). The organic phase was separated and washed with water (2×50 mL) and saturated aqueous NaHCO₃ (50 mL). The organic phase was separated, dried over Na₂SO₄, and concentrated to dryness under reduced pressure. The crude product was purified by column chromatography (SiO₂, 0-50% EtOAc gradient in hexanes) to yield 5-(2-bromo-5-fluorophenyl)-1-methyl-1,2,4-triazole.

Step c: To a solution of 5-(2-bromo-5-fluorophenyl)-1-methyl-1,2,4-triazole (91.5 mg, 0.36 mmol, 1.0 equiv.), 1-(2-chloro-6-cyclopropylpyridin-4-yl)-5-methyl-2,8-dioxa-5-azonia-1-boranuidabicyclo[3.3.0]octane-3,7-dione (110 mg, 0.36 mmol, 1.0 equiv., prepared according to Example 18) and K₃PO₄ (228 mg, 1.1 mmol, 3.0 equiv.) in dioxane (1.8 mL) and water (0.36 mL) mixture was added Pd(dppf)Cl₂ (26 mg, 0.0357 mmol, 0.1 equiv.). The mixture was degassed under vacuum and backfilled with nitrogen (repeated 2 times) and stirred at 90° C. for 1 h. The obtained dark solution was cooled to room temperature, diluted with EtOAc (30 mL) and washed with brine (10 ml). The organic phase was dried over Na₂SO₄, and the solvent was removed under reduced pressure. The crude product was purified by column chromatography (SiO₂, 0-60% EtOAc in hexane) to afford 2-chloro-6-cyclopropyl-4-[4-fluoro-2-(2-methyl-1,2,4-triazol-3-yl)phenyl]pyridine as a yellow solid.

Step d: To a solution of the product from step c (27 mg, 0.082 mmol, 1.0 equiv.) and 4-cyclopropyl-2-[[(3S)-3-methylpiperidin-1-yl]methyl]-1-(2-trimethylsilylethoxymethyl)-6H-pyrrolo[2,3-c]pyridin-7-one (34 mg, 0.082 mmol, 1.0 equiv., obtained according to example 1) in dioxane (1 mL) was added CuI (16 mg, 0.08 mmol, 1.0 equiv.), DMEDA (0.036 mL, 0.33 mmol, 4.0 equiv.) and K₂CO₃ (23 mg, 0.16 mmol, 2.0 equiv.). The resulting mixture was sparged with nitrogen and stirred at 110° C. for 12 h in a sealed vial, then it was quenched with aq. sat. NH₄Cl (2 mL) and diluted with EtOAc (10 mL). The organic phase was separated, and the aqueous layer was extracted with EtOAc (2×5 mL). The combined organics was dried over Na₂SO₄ and concentrated to dryness. The crude coupling product was purified by column chromatography (SiO₂, 0-20% methanol gradient in dichloromethane) to afford 4-cyclopropyl-6-[6-cyclopropyl-4-[4-fluoro-2-(2-methyl-1,2,4-triazol-3-yl)phenyl]pyridin-2-yl]-2-[[(3S)-3-methylpiperidin-1-yl]methyl]-1-(2-trimethylsilylethoxymethyl)pyrrolo[2,3-c]pyridin-7-one.

Step e: To a solution of the product from step d in dichloromethane (2 mL) was added trifluororacetic acid (1 mL). The resulting solution was stirred at 23° C. for 1 h, then solvent was evaporated under vacuum, and the crude residue was dissolved in NH₃ in MeOH (7M, 3 mL). After stirring for 30 min the mixture was concentrated to dryness, and the crude product was purified by prep-HPLC (SiO₂ C18 column, 5 to 50% MeCN in water with 0.1% trifluoroacetic acid) to furnish the title compound. ¹H NMR (400 MHz, DMSO-d₆) δ 11.96 (s, 1H), 7.95 (s, 1H), 7.70 (dd, J=8.6, 5.6 Hz, 1H), 7.63-7.51 (m, 2H), 7.26 (d, J=1.4 Hz, 1H), 6.98 (d, J=1.1 Hz, 1H), 6.82 (d, J=1.4 Hz, 1H), 6.29 (s, 1H), 3.55 (s, 2H), 3.44 (s, 3H), 2.81-2.70 (m, 2H), 2.01 (tt, J=8.4, 4.5 Hz, 1H), 1.86 (qd, J=8.7, 5.1 Hz, 2H), 1.64-1.49 (m, 4H), 1.49-1.36 (m, 1H), 0.97-0.90 (m, 2H), 0.84-0.73 (m, 8H), 0.62-0.54 (m, 2H). ESI MS [M+H]⁺ for C₃₄H₃₇FN₇O, calcd 578.3, found 578.3.

### Example 120: 3-[6-cyclopropyl-4-[4-fluoro-2-(2-methyl-1,2,4-triazol-3-yl)phenyl]-pyridin-2-yl]-6-[[(1-methylcyclobutyl)amino]methyl]-5H-pyrrolo[3,2-d]pyrimidin-4-one

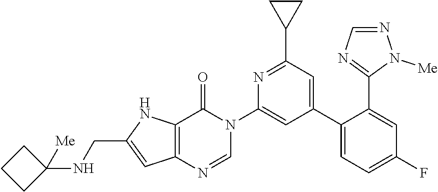 [SMILES: Cn1ncnc1-c1cc(F)ccc1-c1cc(C2CC2)nc(-n2cnc3cc(CNC4(C)CCC4)[nH]c3c2=O)c1]

The title compound was prepared in a similar fashion to that described for example 118 using 6-[[(1-methylcyclobutyl)amino]methyl]-5-(2-trimethylsilylethoxymethyl)-3H-pyrrolo[3,2-d]pyrimidin-4-one (prepared in an analogous manner to that described in example 22) and 2-chloro-6-cyclopropyl-4-[4-fluoro-2-(2-methyl-1,2,4-triazol-3-yl)phenyl]pyridine. ¹H NMR (400 MHz, CDCl₃) δ 8.28 (s, 1H), 7.64 (dd, J=8.7, 5.7 Hz, 1H), 7.46 (dd, J=9.6, 1.2 Hz, 2H), 7.40 (ddd, J=8.7, 7.7, 2.7 Hz, 1H), 7.28 (d, J=2.6 Hz, 1H), 6.62 (d, J=1.4 Hz, 1H), 6.34 (s, 1H), 3.97 (s, 2H), 2.16 (q, J=9.7, 9.1 Hz, 2H), 1.92 (d, J=0.9 Hz, 3H), 1.91-1.74 (m, 3H), 1.38 (s, 3H), 1.03-0.89 (m, 4H). ESI MS [M+H]⁺ for C₂₉H₂₉FN₈O, calcd 525.3, found 525.3.

### Example 121: 6-(cyclopentylmethyl)-3-[6-cyclopropyl-4-[4-fluoro-2-(4-methyl-1,2,4-triazol-3-yl)phenyl]pyridin-2-yl]-5H-pyrrolo[3,2-d]pyrimidin-4-one

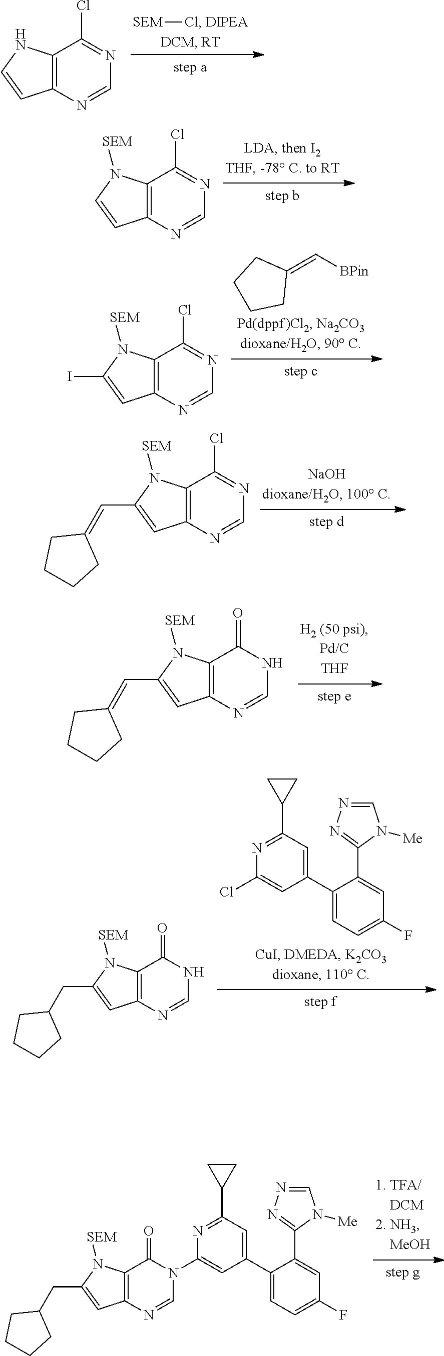

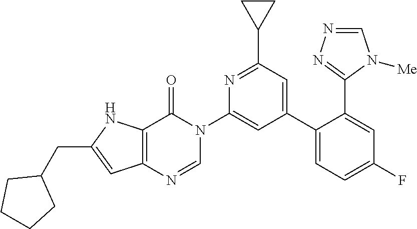 [SMILES: Cn1cnnc1-c1cc(F)ccc1-c1cc(C2CC2)nc(-n2cnc3cc(CC4CCCC4)[nH]c3c2=O)c1]

Step a: To a solution of 4-chloro-5H-pyrrolo[3,2-d]pyrimidine (8.5 g, 55.3 mmol, 1.0 equiv.) in dichloromethane (220 mL) was added DIPEA (14.5 mL, 83.0 mmol, 1.5 equiv.) followed by SEM-Cl (10.8 mL, 60.8 mmol, 1.1 equiv.). The reaction mixture was stirred at room temperature for 1 hour at which point it was quenched with a 1:1 mixture of water/brine (500 mL) and extracted with dichloromathane (2×200 mL). The combined organics were dried over Na₂SO₄, filtered, and concentrated under vacuum. The crude residue was purified by column chromatography (SiO₂, EtOAc in hexanes, 0 to 60%) to afford the desired product.

Step b: LDA (2.0 M in THF, 4.4 mL, 8.8 mmol, 1.25 equiv.) was added to THF (35 mL) and the resulting solution was cooled to −78° C. A solution of the product from step a (2.0 g, 7.0 mmol, 1.0 equiv.) in THF (5 mL) was added dropwise to the reaction mixture over a 5 min period. The reaction mixture was stirred at −78° C. for 1.5 hours, at which point I₂ (2.7 g, 10.6 mmol, 1.5 equiv.) was added dropwise to the reaction mixture as a solution in THE (5 mL). The reaction was stirred for an additional 30 minutes at −78° C. The cooling bath was removed, and the reaction was allowed to warm to room temperature and stir for 30 minutes. The resulting mixture was quenched with saturated aqueous NH₄Cl (20 mL) and partitioned between EtOAc (100 mL) and H₂O (150 mL). The aqueous phase was extracted with EtOAc (100 mL), and the combined organics were dried over Na₂SO₄, filtered, and concentrated under vacuum. The crude residue was purified by column chromatography (SiO₂, EtOAc in hexanes, 0 to 100%) to afford the desired product.

Step c: To a solution of the product from step b (250 mg, 0.61 mmol, 1.0 equiv.), and 2-(cyclopentylidenemethyl)-4,4,5,5-tetramethyl-1,3,2-dioxaborolane (152 mg, 0.73 mmol, 1.2 equiv.) in dioxane (6 mL) was added 1.0 M aqueous Na₂CO₃ (1.82 mL). The reaction mixture was sparged with N₂ for 10 minutes. Pd(dppf)Cl₂ (44 mg, 0.06 mmol, 0.1 equiv.) was added and the reaction mixture was head to 90° C. and stirred for 1.5 hour at which point it was quenched with a 1:1 mixture of water/brine (100 mL) and extracted with EtOAc (2×50 mL). The combined organics were dried over Na₂SO₄, filtered, and concentrated under vacuum. The crude residue was purified by column chromatography (SiO₂, EtOAc in hexanes, 0 to 60%) to afford the desired product.

Step d: To a solution of the product from step c (167 mg, 0.46 mmol, 1.0 equiv.) in dioxane (2.5 mL) was added 1.0 M aqueous NaOH (2.5 mL). The reaction mixture was heated to 100° C. and stirred for 16 hours at which point it was quenched with saturated aqueous NH₄Cl (20 mL) and extracted with EtOAc (2×10 mL). The combined organics were dried over Na₂SO₄, filtered, and concentrated under vacuum. The crude residue was used without any further purification.

Step e: A Parr shaker was charged with the crude product from step d (assume 0.46 mmol, 1.0 equiv.) in EtOAc (10 mL). Pd/C (200 mg, 10 wt % Pd) was added and the Parr shaker was evacuated/backfilled with 50 psi of H₂ three times. The reaction mixture was shaken under 50 psi H₂ for 72 hours at which point it was filtered through a Celite® pad to remove solids. The filtrate was concentrated under vacuum, and the crude residue was used without any further purification.

Step f: To a suspension of the product from step e (74 mg, 0.21 mmol, 1.0 equiv.), 2-chloro-6-cyclopropyl-4-[4-fluoro-2-(4-methyl-1,2,4-triazol-3-yl)phenyl]pyridine (69 mg, 0.21 mmol, 1.0 equiv., prepared according to example 28), and K₂CO₃ (87 mg, 0.63 mmol, 3.0 equiv.) in dioxane (4.2 mL) was added CuI (40 mg, 0.21 mmol, 1.0 equiv.) and DMEDA (45 μL, 0.42 mmol, 2.0 equiv.). The reaction mixture was heated to 110° C. and stirred for 16 hours at which point it was quenched with a 1:1 mixture of water/brine (20 mL) and extracted with EtOAc (2×10 mL). The combined organics were dried over Na₂SO₄, filtered, and concentrated under vacuum. The crude residue was purified by column chromatography (SiO₂, MeOH in dichloromethane, 0 to 20%) to afford the desired product.

Step g: To a solution of the product from step f (34 mg, 0.05 mmol, 1.0 equiv.) in dichloromethane (1 mL) was added trifluoroacetic acid (1 mL). The reaction mixture was heated to 30° C. and stirred for 1 hour at which point it was diluted with toluene (5 mL) and concentrated under vacuum. The crude residue was dissolved in 7M NH₃ in MeOH (2 mL), and the resulting reaction mixture was stirred at 30° C. for 1 hour. The reaction was directly concentrated and purified by reverse phase prep-HPLC (SiO₂ C18 column, 5 to 60% MeCN gradient in water with 0.1% trifluoroacetic acid) to afford the desired product. ¹H NMR (400 MHz, CDCl₃) δ 9.17 (s, 1H), 8.27 (s, 1H), 8.12 (s, 1H), 7.59 (dd, J=8.6, 5.3 Hz, 1H), 7.44-7.32 (m, 3H), 6.93 (d, J=1.4 Hz, 1H), 6.30 (d, J=2.3 Hz, 1H), 3.15 (s, 3H), 2.74 (d, J=7.5 Hz, 2H), 2.19 (p, J=7.7 Hz, 1H), 2.01-1.85 (m, 1H), 1.85-1.74 (m, 2H), 1.72-1.37 (m, 3H), 1.30-1.15 (m, 3H), 1.07-0.88 (m, 4H). ESI MS [M+H]⁺ for C₂₉H₂₈FN₇O, calcd 510.2, found 510.1.

### Example 122: 3-[6-cyclopropyl-4-[4-fluoro-2-(4-methyl-1,2,4-triazol-3-yl)phenyl]pyridin-2-yl]-6-(oxan-4-ylmethyl)-5H-pyrrolo[3,2-d]pyrimidin-4-one

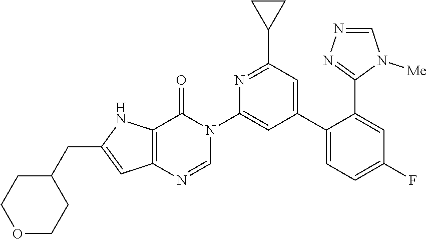 [SMILES: Cn1cnnc1-c1cc(F)ccc1-c1cc(C2CC2)nc(-n2cnc3cc(CC4CCOCC4)[nH]c3c2=O)c1]

The title compound was prepared in a similar fashion to that described for example 121 using 4,4,5,5-tetramethyl-2-(oxan-4-ylidenemethyl)-1,3,2-dioxaborolane for step c. ¹H NMR (400 MHz, CDCl₃) δ 9.40 (s, 1H), 8.26 (s, 1H), 8.17 (s, 1H), 7.60 (dd, J=8.5, 5.3 Hz, 1H), 7.44-7.35 (m, 2H), 7.30 (d, J=1.3 Hz, 1H), 7.01 (s, 1H), 6.29 (d, J=2.3 Hz, 1H), 3.96 (dd, J=11.6, 4.4 Hz, 2H), 3.40-3.28 (m, 2H), 3.16 (s, 3H), 2.68 (d, J=7.2 Hz, 2H), 2.06-1.79 (m, 2H), 1.46-1.28 (m, 2H), 1.06-0.89 (m, 4H). ESI MS [M+H]+ for C₂₉H₂₈FN₇O2, calcd 526.2, found 526.2.

### Example 123: 3-[6-cyclopropyl-4-[4-fluoro-2-(4-methyl-1,2,4-triazol-3-yl)phenyl]pyridin-2-yl]-6-(oxetan-3-ylmethyl)-5H-pyrrolo[3,2-d]pyrimidin-4-one

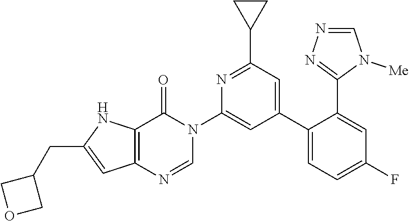 [SMILES: Cn1cnnc1-c1cc(F)ccc1-c1cc(C2CC2)nc(-n2cnc3cc(CC4COC4)[nH]c3c2=O)c1]

The title compound was prepared in a similar fashion to that described for example 121 using 4,4,5,5-tetramethyl-2-(oxetan-3-ylidenemethyl)-1,3,2-dioxaborolane for step c. ¹H NMR (400 MHz, CDCl₃) δ 10.03 (s, 1H), 8.24 (s, 1H), 8.21 (s, 1H), 7.60 (dd, J=8.6, 5.4 Hz, 1H), 7.45-7.33 (m, 2H), 7.30 (d, J=1.4 Hz, 1H), 7.03 (d, J=1.4 Hz, 1H), 6.22 (d, J=2.2 Hz, 1H), 4.83 (dd, J=7.6, 6.2 Hz, 2H), 4.44 (t, J=6.1 Hz, 2H), 3.43-3.31 (m, 1H), 3.15 (s, 3H), 3.13 (d, J=7.9 Hz, 2H), 2.05-1.94 (m, 1H), 1.92-1.74 (m, 4H), 1.10-0.93 (m, 4H). ESI MS [M+H]⁺ for C₂₇H₂₄FN₇O₂, calcd 498.2, found 498.2.

### Example 124: 3-[6-cyclopropyl-4-[4-fluoro-2-(4-methyl-1,2,4-triazol-3-yl)phenyl]-pyridin-2-yl]-6-methyl-5H-pyrrolo[3,2-d]pyrimidin-4-one

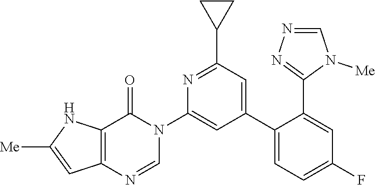 [SMILES: Cc1cc2ncn(-c3cc(-c4ccc(F)cc4-c4nncn4C)cc(C4CC4)n3)c(=O)c2[nH]1]

The title compound was prepared in a similar fashion to that described for example 121 using methylboronic acid for step c. ¹H NMR (400 MHz, CDCl₃) δ 9.27 (s, 1H), 8.27 (s, 1H), 8.14 (s, 1H), 7.59 (dd, J=8.6, 5.4 Hz, 1H), 7.45-7.34 (m, 3H), 6.94 (d, J=1.5 Hz, 1H), 6.28 (dd, J=2.4, 0.9 Hz, 1H), 3.15 (s, 3H), 2.44 (d, J=0.8 Hz, 3H), 2.02-1.93 (m, 1H), 1.04-0.88 (m, 4H). ESI MS [M+H]+ for C₂₄H₂₀FN₇O, calcd 442.2, found 442.1.

### Example 125: 3-[6-cyclopropyl-4-[4-fluoro-2-(4-methyl-1,2,4-triazol-3-yl)phenyl]-pyridin-2-yl]-6-(oxolan-3-yl)-5H-pyrrolo[3,2-d]pyrimidin-4-one

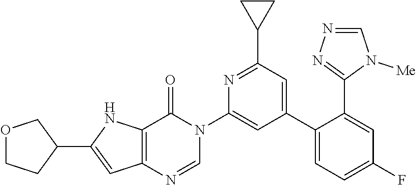 [SMILES: Cn1cnnc1-c1cc(F)ccc1-c1cc(C2CC2)nc(-n2cnc3cc(C4CCOC4)[nH]c3c2=O)c1]

The title compound was prepared in a similar fashion to that described for example 121 using 2-(2,5-dihydrofuran-3-yl)-4,4,5,5-tetramethyl-1,3,2-dioxaborolane for step c. ¹H NMR (400 MHz, CDCl₃) δ 9.56 (s, 1H), 8.27 (s, 1H), 8.14 (s, 1H), 7.59 (dd, J=8.6, 5.4 Hz, 1H), 7.44-7.34 (m, 2H), 7.33 (d, J=1.4 Hz, 1H), 6.97 (d, J=1.4 Hz, 1H), 6.34 (d, J=2.1 Hz, 1H), 4.13-3.99 (m, 2H), 3.95-3.83 (m, 2H), 3.60-3.55 (m, 1H), 3.15 (s, 3H), 2.49-2.35 (m, 1H), 2.13-2.02 (m, 1H), 2.02-1.87 (m, 1H), 1.04-0.90 (m, 4H). ESI MS [M+H]+ for C₂₇H₂₄FN₇O₂, calcd 498.2, found 498.1.

### Example 126: 6-[cyclopentyl(hydroxy)methyl]-3-[6-cyclopropyl-4-[4-fluoro-2-(4-methyl-1,2,4-triazol-3-yl)phenyl]pyridin-2-yl]-5H-pyrrolo[3,2-d]pyrimidin-4-one

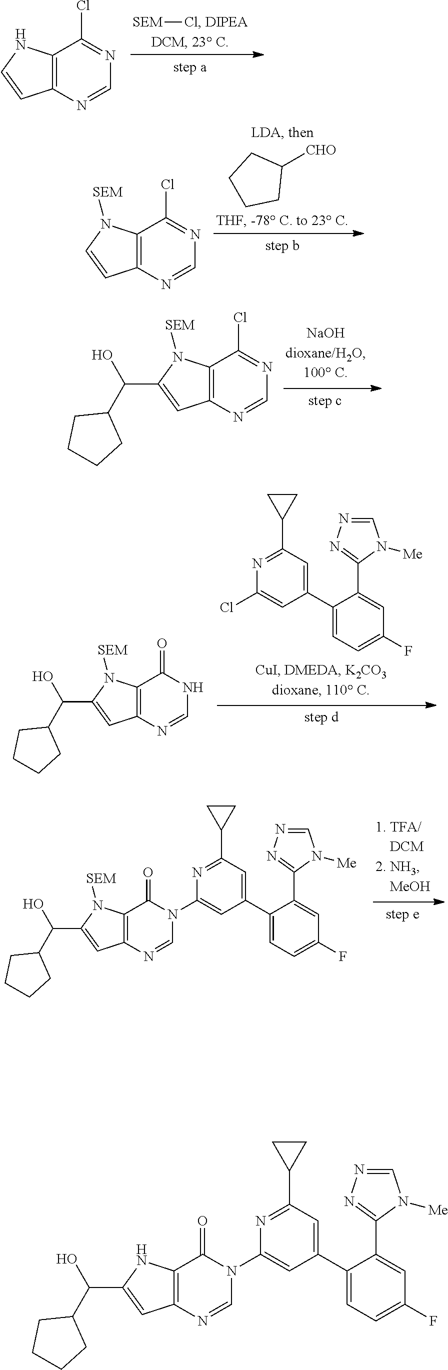

Step a: To a solution of 4-chloro-5H-pyrrolo[3,2-d]pyrimidine (8.5 g, 55.3 mmol, 1.0 equiv.) in dichloromethane (220 mL) was added DIPEA (14.5 mL, 83.0 mmol, 1.5 equiv.) followed by SEM-Cl (10.8 mL, 60.8 mmol, 1.1 equiv.). The reaction mixture was stirred at room temperature for 1 hour at which point it was quenched with a 1:1 mixture of water/brine (500 mL) and extracted with dichloromethane (200 mL). The combined organics were dried over Na₂SO₄, filtered, and concentrated under vacuum. The crude residue was purified by column chromatography (SiO₂, EtOAc in hexanes, 0 to 60%) to afford the desired product.

Step b: LDA (2.0 M in THF, 4.4 mL, 8.8 mmol, 1.25 equiv.) was added to THF (27 mL) and the resulting solution was cooled to −78° C. The product of step a (2.0 g, 7.05 mmol, 1.0 equiv.) was added dropwise to the reaction mixture as a solution in THF (5 mL). The reaction mixture was stirred at −78° C. for 1.5 hours, at which point cyclopentanecarbaldehyde (1.0 g, 10.6 mmol, 1.5 equiv.) was added dropwise to the reaction mixture. The reaction was stirred for an additional 30 minutes at −78° C. The dry ice bath was removed, and the reaction was allowed to warm to room temperature and stir for 30 minutes. The reaction was quenched with saturated aqueous NH₄Cl (20 mL) and partitioned between EtOAc (100 mL) and H₂O (100 mL). The aqueous phase was extracted with EtOAc (100 mL). The combined organics were dried over Na₂SO₄, filtered, and concentrated under vacuum. The crude residue was purified by column chromatography (SiO₂, EtOAc in hexanes, 0 to 80%) to afford the desired product.

Step c: To a solution of the product of step b (375 mg, 1.0 mmol, 1.0 equiv.) in dioxane (5 mL) was added 1.0 M aqueous NaOH (5 mL). The reaction mixture was heated to 100° C. and stirred for 16 hours at which point it was quenched with saturated aqueous NH₄Cl (150 mL) and extracted with EtOAc (2×100 mL). The combined organics were dried over Na₂SO₄, filtered, and concentrated under vacuum. The crude residue was used without any further purification.

Step d: To a suspension of the product from step c (100 mg, 0.28 mmol, 1.0 equiv.), 2-chloro-6-cyclopropyl-4-[4-fluoro-2-(4-methyl-1,2,4-triazol-3-yl)phenyl]pyridine (92 mg, 0.28 mmol, 1.0 equiv., prepared according to example 28), and K₂CO₃ (115 mg, 0.83 mmol, 3.0 equiv.) in dioxane (5.5 mL) was added CuI (53 mg, 0.28 mmol, 1.0 equiv.) and DMEDA (60 uL, 0.56 mmol, 2.0 equiv.). The reaction mixture was heated to 110° C. and stirred for 16 hours at which point it was quenched with a 1:1 mixture of water/brine (100 mL) and extracted with EtOAc (2×50 mL). The combined organics were dried over Na₂SO₄, filtered, and concentrated under vacuum. The crude residue was purified by column chromatography (SiO₂, MeOH in dichloromethane, 0 to 20%) to afford the desired product.

Step e: To a solution of the product from step d (13 mg, 0.02 mmol, 1.0 equiv.) in dichloromethane (1 mL) was added trifluoroacetic acid (1 mL). The reaction mixture was heated to 30° C. and stirred for 1 hour at which point it was diluted with toluene (5 mL) and concentrated under vacuum. The crude residue was dissolved in 7M NH₃ in MeOH (2 mL) and the resulting reaction mixture was stirred at 30° C. for 1 hour. The reaction was directly concentrated and purified by reverse phase prep-HPLC (SiO₂ C18 column, 5 to 60% MeCN in water with 0.1% trifluoroacetic acid) to afford the desired product. ¹H NMR (400 MHz, CDCl₃) δ 10.16 (s, 1H), 8.27 (s, 1H), 8.14 (s, 1H), 7.61 (dd, J=8.6, 5.4 Hz, 1H), 7.43-7.34 (m, 3H), 6.97 (d, J=1.3 Hz, 1H), 6.36 (d, J=2.0 Hz, 1H), 4.73 (d, J=7.2 Hz, 1H), 3.16 (s, 3H), 2.29 (q, J=7.9 Hz, 1H), 2.06-1.90 (m, 1H), 1.84-1.40 (m, 6H), 1.28 (d, J=26.6 Hz, 2H), 1.05-0.91 (m, 5H). ESI MS [M+H]+ for C₂₉H₂₈FN₇O₂, calcd 526.2, found 526.1.

### Example 127: 6-[cyclopentyl(fluoro)methyl]-3-[6-cyclopropyl-4-[4-fluoro-2-(4-methyl-1,2,4-triazol-3-yl)phenyl]pyridin-2-yl]-5H-pyrrolo[3,2-d]pyrimidin-4-one

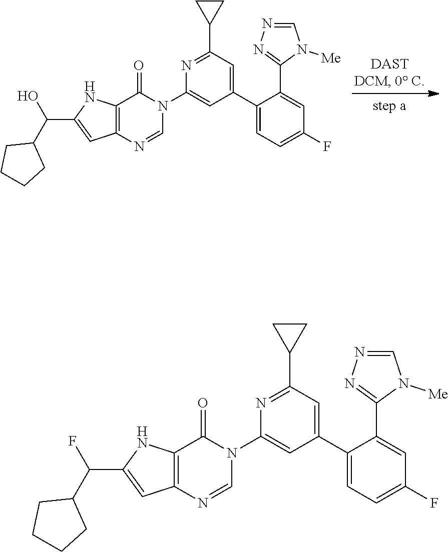 [SMILES: Cn1cnnc1-c1cc(F)ccc1-c1cc(C2CC2)nc(-n2cnc3cc(C(O)C4CCCC4)[nH]c3c2=O)c1 ; Cn1cnnc1-c1cc(F)ccc1-c1cc(C2CC2)nc(-n2cnc3cc(C(F)C4CCCC4)[nH]c3c2=O)c1]

Step a: To a solution of 6-[cyclopentyl(hydroxy)methyl]-3-[6-cyclopropyl-4-[4-fluoro-2-(4-methyl-1,2,4-triazol-3-yl)phenyl]pyridin-2-yl]-5H-pyrrolo[3,2-d]pyrimidin-4-one (16 mg, 0.03 mmol, 1.0 equiv., prepared according to example 126) in dichloromethane (1.5 mL) at 0° C. was added DAST (12 μL, 0.09 mmol, 3.0 equiv.). The reaction mixture was stirred at 0° C. for 1 hour at which point it was quenched into saturated aqueous NaHCO₃ (20 mL) and extracted with dichloromethane (2×10 mL). The combined organics were dried over Na₂SO₄, filtered, and concentrated under vacuum. The crude residue was purified by reverse phase prep-HPLC (SiO₂ C18 column, 10 to 70% MeCN in water with 0.1% trifluoroacetic acid) to afford the desired product. ¹H NMR (400 MHz, CDCl₃) δ 9.65 (s, 1H), 8.30 (s, 1H), 8.13 (s, 1H), 7.59 (dd, J=8.5, 5.4 Hz, 1H), 7.45-7.33 (m, 3H), 6.97 (d, J=1.4 Hz, 1H), 6.47 (t, J=2.2 Hz, 1H), 5.39 (dd, J=47.4, 7.6 Hz, 1H), 3.16 (s, 3H), 2.49 (dq, J=16.3, 7.8 Hz, 1H), 2.04-1.94 (m, 1H), 1.91-1.51 (m, 7H), 1.38-1.27 (m, 1H), 1.03-0.91 (m, 4H). ESI MS [M+H]⁺ for C₂₉H₂₇F₂N₇O, calcd 528.2, found 528.1.

### Example 128: 3-[6-cyclopropyl-4-[4-fluoro-2-(4-methyl-1,2,4-triazol-3-yl)phenyl]-pyridin-2-yl]-6-(difluoromethyl)-5H-pyrrolo[3,2-d]pyrimidin-4-one

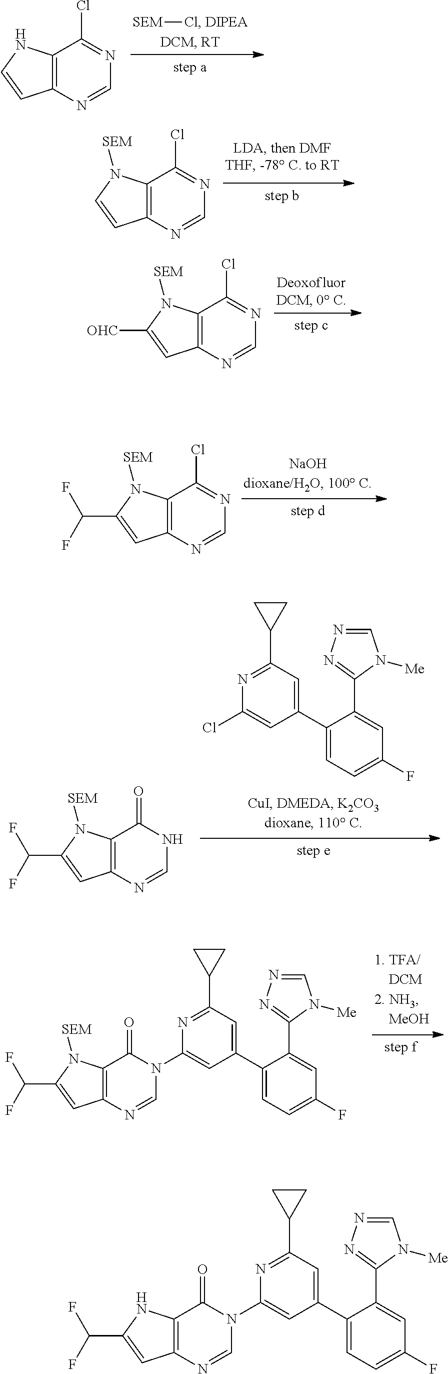

Step a: To a solution of 4-chloro-5H-pyrrolo[3,2-d]pyrimidine (8.5 g, 55.3 mmol, 1.0 equiv.) in dichloromethane (220 mL) was added DIPEA (14.5 mL, 83.0 mmol, 1.5 equiv.) followed by SEM-Cl (10.8 mL, 60.8 mmol, 1.1 equiv.). The reaction mixture was stirred at room temperature for 1 hour at which point it was quenched with a 1:1 mixture of water/brine (500 mL) and extracted with dichloromethane (200 mL). The combined organics were dried over Na₂SO₄, filtered, and concentrated under vacuum. The crude residue was purified by column chromatography (SiO₂, EtOAc in hexanes, 0 to 60%) to afford the desired product.

Step b: LDA (2.0 M in THF, 3.3 mL, 6.0 mmol, 1.25 equiv.) was added to THF (27 mL) and the resulting solution was cooled to −78° C. The product of step a (1.5 g, 5.3 mmol, 1.0 equiv.) was added dropwise to the reaction mixture as a solution in THE (5 mL). The reaction mixture was stirred at −78° C. for 1.5 hours, at which point DMF (0.65 mL, 7.9 mmol, 1.5 equiv.) was added dropwise to the reaction mixture over 1 min period. The reaction was stirred for an additional 30 min at −78° C. The dry ice bath was removed, and the reaction was allowed to warm to room temperature and stir for 30 minutes. The reaction was quenched with saturated aqueous NH₄Cl (20 mL) and partitioned between EtOAc (100 mL) and H₂O (100 mL). The aqueous phase was extracted with EtOAc (100 mL), and the combined organics were dried over Na₂SO₄, filtered, and concentrated under vacuum. The crude residue was purified by column chromatography (SiO₂, EtOAc in hexanes, 0 to 100%) to afford the desired product.

Step c: To a solution of the product of step b (100 mg, 0.32 mmol, 1.0 equiv.) in dichloromethane (3 mL) at 0° C. was added DeoxoFluor® (0.80 mL, 1.60 mmol, 5.0 equiv.). The reaction mixture was stirred at 0° C. for 1 hour at which point it was quenched into saturated aqueous NaHCO₃ (20 mL) and extracted with dichloromethane (2×10 mL). The combined organics were dried over Na₂SO₄, filtered, and concentrated under vacuum. The crude residue was purified by column chromatography (SiO₂, EtOAc in hexanes, 0 to 60%) to afford the desired product.

Step d: To a solution of the product of step c (45 mg, 0.14 mmol, 1.0 equiv.) in dioxane (1.5 mL) was added 1.0 M aqueous NaOH (1.5 mL). The reaction mixture was heated to 100° C. and stirred for 16 hours at which point it was quenched with saturated aqueous NH₄Cl (20 mL) and extracted with EtOAc (2×10 mL). The combined organics were dried over Na₂SO₄, filtered, and concentrated under vacuum. The crude residue was used without any further purification.

Step e: To a suspension of the product from step d (42 mg, 0.13 mmol, 1.0 equiv.), 2-chloro-6-cyclopropyl-4-[4-fluoro-2-(4-methyl-1,2,4-triazol-3-yl)phenyl]pyridine (43 mg, 0.13 mmol, 1.0 equiv., prepared according to example 28), and K₂CO₃ (54 mg, 0.39 mmol, 3.0 equiv.) in dioxane (3 mL) was added CuI (25 mg, 0.13 mmol, 1.0 equiv.) and DMEDA (28 uL, 0.26 mmol, 2.0 equiv.). The reaction mixture was heated to 110° C. and stirred for 16 hours at which point it was quenched with a 1:1 mixture of water/brine (20 mL) and extracted with EtOAc (2×10 mL). The combined organics were dried over Na₂SO₄, filtered, and concentrated under vacuum. The crude residue was purified by column chromatography (SiO₂, MeOH in dichloromethane, 0 to 20%) to afford the desired product.

Step f: To a solution of the product from step e (55 mg, 0.09 mmol, 1.0 equiv.) in dichloromethane (1 mL) was added TFA (1 mL). The reaction mixture was heated to 30° C. and stirred for 1 hour at which point it was diluted with toluene (5 mL) and concentrated under vacuum. The crude residue was dissolved in 7M NH₃ in MeOH (2 mL) and the resulting reaction mixture was stirred at 30° C. for 1 hour. The reaction was directly concentrated and purified by reverse phase prep-HPLC (C18 column, 5 to 60% MeCN in water with 0.1% trifluoracetic acid) to afford the desired product. ¹H NMR (400 MHz, DMSO-d6) δ 13.26-12.96 (m, 1H), 8.50 (s, 1H), 8.22 (s, 1H), 7.77 (dd, J=8.6, 5.6 Hz, 1H), 7.68-7.55 (m, 2H), 7.32-6.96 (m, 3H), 6.78 (d, J=1.8 Hz, 1H), 3.29 (s, 3H), 2.12-2.01 (m, 1H), 1.03-0.95 (m, 2H), 0.92-0.83 (m, 2H). ESI MS [M+H]+ for C₂₄H₁₈F₃N₇O, calcd 478.2, found 478.2.

### Example 129: 4-[2-cyclopropyl-6-[6-[[(3S)-3-methylpiperidin-1-yl]methyl]-4-oxo-5H-pyrrolo[3,2-d]pyrimidin-3-yl]pyridin-4-yl]-3-(1,4-dimethylpyrazol-3-yl)benzonitrile and 4-[2-cyclopropyl-6-[6-[[(3S)-3-methylpiperidin-1-yl]methyl]-4-oxo-5H-pyrrolo[3,2-d]pyrimidin-3-yl]pyridin-4-yl]-3-(2,4-dimethylpyrazol-3-yl)benzonitrile

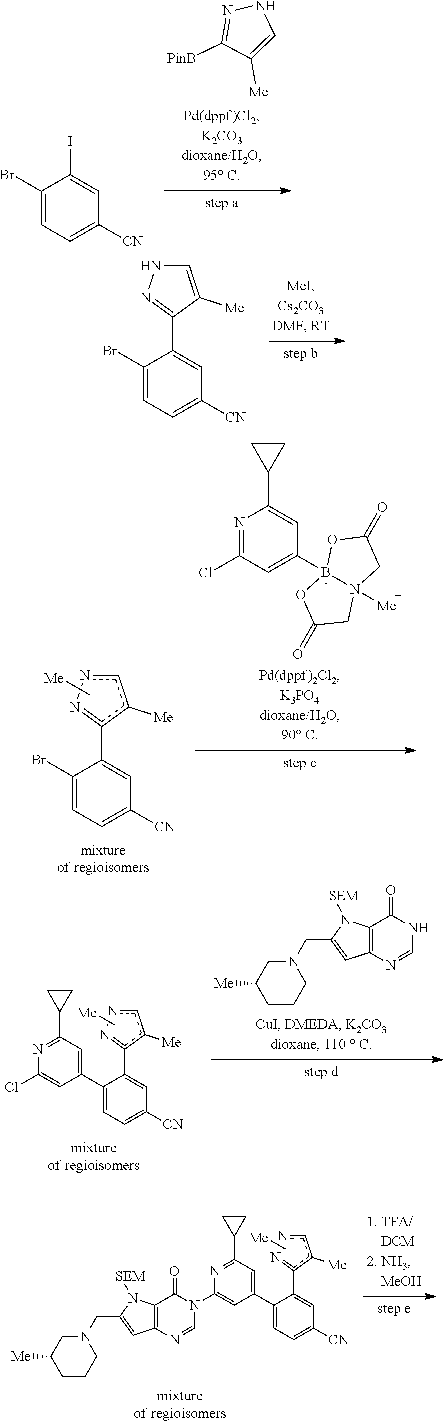

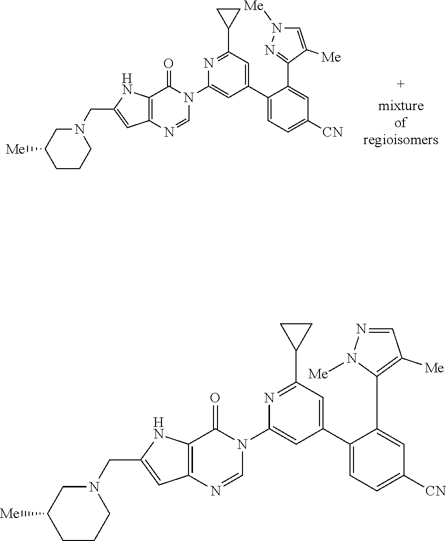 [SMILES: Cc1cn(C)nc1-c1cc(C#N)ccc1-c1cc(C2CC2)nc(-n2cnc3cc(CN4CCC[C@H](C)C4)[nH]c3c2=O)c1 ; Cc1cnn(C)c1-c1cc(C#N)ccc1-c1cc(C2CC2)nc(-n2cnc3cc(CN4CCC[C@H](C)C4)[nH]c3c2=O)c1]

Step a: A suspension of 4-bromo-3-iodobenzonitrile (1.43 g, 4.64 mmol, 1.0 equiv.), 4-methyl-3-(4,4,5,5-tetramethyl-1,3,2-dioxaborolan-2-yl)-1H-pyrazole (970 mg, 4.64 mmol, 1.0 equiv.), and K₂CO₃ (1.92 g, 13.9 mmol, 3.0 equiv.) in a 3:1 mixture of dioxane/water (30 mL) was sparged with N₂ for 10 minutes. Pd(dppf)Cl₂ (336 mg, 0.46 mmol, 0.1 equiv.) was added and the reaction mixture was heated to 95° C. and stirred for 16 hours at which point it was quenched with a 1:1 mixture was water and brine (200 mL) and extracted with EtOAc (2×100 mL). The combined organics were dried over Na₂SO₄, filtered, and concentrated under vacuum. The crude residue was purified by column chromatography (SiO₂, EtOAc in hexanes, 0 to 100%) to afford the desired product.

Step b: To a suspension of the product from step a (50 mg, 0.19 mmol, 1.0 equiv.) and Cs₂CO₃ (124 mg, 0.38 mmol, 2.0 equiv.) in DMF (2 mL) was added MeI (15 μL, 0.23 mmol, 1.2 equiv.). The reaction mixture was stirred for 16 hours at room temperature at which point it was quenched with water (20 mL) and extracted with EtOAc (2×10 mL). The combined organics were dried over Na₂SO₄, filtered, and concentrated under vacuum. The crude residue was purified by column chromatography (SiO₂, EtOAc in hexanes, 0 to 100%) to afford the desired product as a ˜1:1 mixture of regioisomers.

Step c: A round-bottomed flask was charged with the product from step b (35 mg, 0.13 mmol, 1.0 equiv.), 1-(2-chloro-6-cyclopropylpyridin-4-yl)-5-methyl-2,8-dioxa-5lambda5-aza-1-boranuidabicyclo[3.3.0]octane-3,7-dione (40 mg, 0.13 mmol, 1.0 equiv., prepared according to example 28), K₃PO₄ (83 mg, 0.39 mmol, 3.0 equiv.), and a 4:1 mixture of dioxane/water (1.5 mL). The resulting suspension was sparged with N₂ for 10 minutes. Pd(dppf)Cl₂ (10 mg, 0.01 mmol, 0.1 equiv.) was added and the reaction mixture was heated to 90° C. and stirred for 1 hour. The reaction was quenched with a 1:1 mixture of water and brine (20 mL) and extracted with EtOAc (2×10 mL). The combined organics were dried over Na₂SO₄, filtered, and concentrated under vacuum. The crude residue was purified by column chromatography (SiO₂, EtOAc in hexanes, 0 to 100%) to afford the desired product as a ˜1:1 mixture of regioisomers.

Step d: To a suspension of the product from step c (35 mg, 0.10 mmol, 1.0 equiv.), 6-[[(3S)-3-methylpiperidin-1-yl]methyl]-5-(2-trimethylsilylethoxymethyl)-3H-pyrrolo[3,2-d]pyrimidin-4-one (38 mg, 0.10 mmol, 1.0 equiv., prepared according to Example 22), and K₂CO₃ (42 mg, 0.30 mmol, 3.0 equiv.) in dioxane (2 mL) was added CuI (20 mg, 0.10 mmol, 1.0 equiv.) and DMEDA (22 uL, 0.20 mmol, 2.0 equiv.). The reaction mixture was heated to 110° C. in a sealed vial and stirred for 16 hours at which point it was quenched with a 1:1 mixture of water/brine (20 mL) and extracted with EtOAc (2×10 mL). The combined organics were dried over Na₂SO₄, filtered, and concentrated under vacuum. The crude residue was purified by column chromatography (SiO₂, EtOAc in hexanes, 0 to 100%) to afford the desired product as a ˜1:1 mixture of regioisomers.

Step e: To a solution of the product from step d (25 mg, 0.04 mmol, 1.0 equiv.) in dichloromethane (1 mL) was added trifluoroacetic acid (1 mL). The reaction mixture was heated to 30° C. and stirred for 1 hour at which point it was diluted with toluene (5 mL) and concentrated under vacuum. The crude residue was dissolved in 7M NH₃ in MeOH (2 mL) and the resulting reaction mixture was stirred at 30° C. for 1 hour. The reaction was directly concentrated and purified by reverse phase prep-HPLC (C18 column, 10 to 70% MeCN in water with 0.1% trifluoroacetic acid) to afford the desired product as an inseparable mixture of regioisomers in ˜1:1 ratio.

Regioisomer (Regio-1): ¹H NMR (400 MHz, CDCl₃) δ 8.33 (s, 1H), 7.86 (dd, J=8.1, 1.7 Hz, 1H), 7.82 (d, J=1.7 Hz, 1H), 7.72 (d, J=8.0 Hz, 1H), 7.50 (d, J=1.4 Hz, 1H), 7.32 (s, 1H), 6.93 (d, J=1.3 Hz, 1H), 6.36 (s, 1H), 3.88 (s, 3H), 3.60 (s, 2H), 2.87-2.65 (m, 2H), 2.02-1.88 (m, 2H), 1.86 (d, J=1.3 Hz, 3H), 1.77-1.61 (m, 4H), 1.02-0.87 (m, 4H), 0.85 (d, J=5.5 Hz, 3H). ESI MS [M+H]+ for C₃₃H₃₄N₈O, calcd 559.3, found 559.2.

Regioisomer (Regio-2): ¹H NMR (400 MHz, CDCl₃) δ 8.22 (s, 1H), 7.75 (dd, J=8.0, 1.8 Hz, 1H), 7.68 (d, J=1.7 Hz, 1H), 7.61 (d, J=8.0 Hz, 1H), 7.45 (d, J=1.4 Hz, 1H), 7.12 (s, 1H), 6.67 (d, J=1.4 Hz, 1H), 6.35 (s, 1H), 3.60 (s, 2H), 3.45 (d, J=1.4 Hz, 3H), 2.87-2.65 (m, 2H), 2.02-1.88 (m, 2H), 1.77-1.61 (m, 7H), 1.25 (s, 3H), 1.02-0.87 (m, 4H), 0.85 (d, J=5.5 Hz, 3H). ESI MS [M+H]+ for C₃₃H₃₄N₈O, calcd 559.3, found 559.2.

### Example 130: 6-[6-cyclopropyl-4-[4-fluoro-2-(5-methyltriazol-1-yl)phenyl]pyridin-2-yl]-2-[(methoxymethylamino)methyl]-4-(trifluoromethyl)-3a,4,5,7a-tetrahydro-1H-pyrrolo[2,3-c]pyridin-7-one

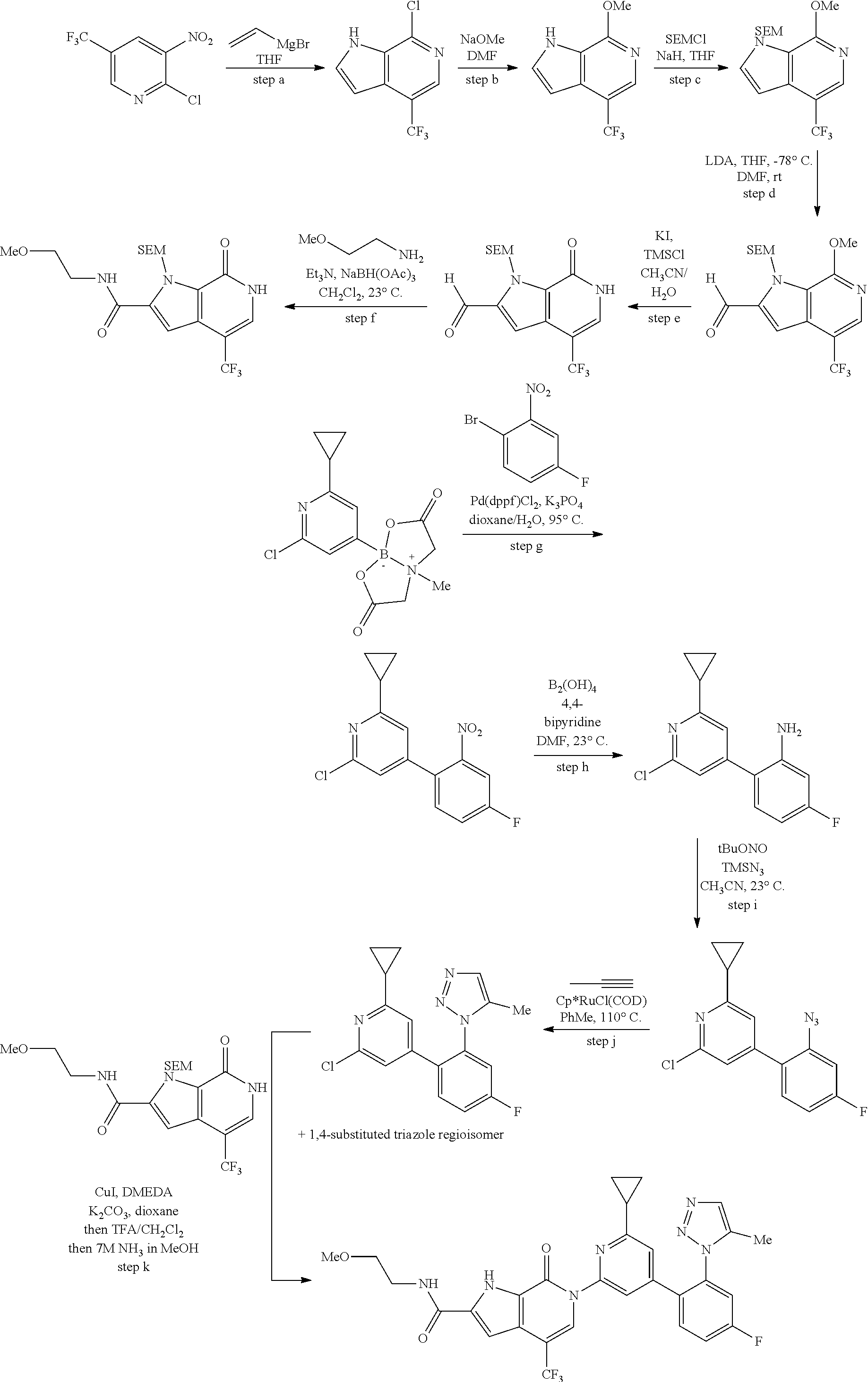

Step a: To a solution of 2-chloro-3-nitro-5-(trifluoromethyl)pyridine (5.3 g, 23 mmol, 1.0 equiv.) in THE (50 mL) was added vinylmagnesium bromide (1M in THF, 76 mL, 76 mmol, 3.3 equiv.) at −78° C. over 30 min. The resulting mixture was stirred at this temperature for another 30 min before being quenched with saturated NH₄Cl aqueous solution (20 mL). The mixture was then extracted with EtOAc (2×80 mL). The combined organic phase was washed with brine, dried over Na₂SO₄ and concentrated. The residue was then purified by column chromatography (SiO₂, EtOAc in hexanes, 0 to 25%) to afford the condensation product.

Step b: To a solution of the product from step a (1.02 g, 4.46 mmol, 1.0 equiv.) in DMF (22 mL) was added NaOMe (2.70 g, 44.6 mmol, 10 equiv.). The resulting mixture was heated at 130° C. for 1 h. After cooling back to room temperature, the mixture was diluted with EtOAc (30 mL) and washed sequentially with water (2×60 mL) and brine (60 mL). The organic phase was then dried over Na₂SO₄ and concentrated. The residue was then purified by column chromatography (SiO₂, EtOAc in hexanes, 0 to 20%) to yield the corresponding product.

Step c: To a solution of a product from step b (772 mg, 3.57 mmol, 1.0 equiv.) in THE (18 mL, 0.2 M) was added NaH (280 mg, 60% in mineral oil, 7.14 mmol, 2.0 equiv.) at 0° C. After 15 minutes, SEM-Cl (890 mg, 5.35 mmol, 1.5 equiv.) was added to the reaction mixture and stirred for 12 h at room temperature. The reaction mixture was quenched with water (10 mL), and the product was extracted with EtOAc (2×20 mL). The combined organic phase was dried over Na₂SO₄, concentrated and the crude residue was purified by column chromatography (SiO₂, EtOAc in hexanes, 10 to 70%) to furnish SEM-protected compound.

Step d: To a solution of the product from step c (1.1 g, 3.2 mmol, 1.0 equiv.) in THF (11 mL, 0.3 M) at −78° C. was added LDA (2.0 M, 1.90 mL, 3.84 mmol, 1.2 equiv.). The mixture was stirred for 30 min at −78° C., then DMF (0.35 g, 4.8 mmol, 1.5 equiv.) was added in one portion. The reaction was stirred at −78° C. for 15 min and warmed up to room temperature and stirred for 2 h. The resulting mixture was quenched with saturated NH₄Cl aqueous solution (7 mL), and the product was extracted with EtOAc (2×10 mL). The combined organic phase was washed with brine, dried over Na₂SO₄ and concentrated. The residue was then purified by column chromatography (SiO₂, EtOAc in hexanes, 0 to 60%) to yield corresponding aldehyde product.

Step e: The product from step d (0.96 g, 2.56 mmol, 1.0 equiv.) was dissolved in MeCN (22 mL, 0.2 M) and H₂O (0.14 mL, 7.68 mmol, 3.0 equiv) followed by the addition of KI (672 mg, 4.04 mmol, 1.6 equiv.) and TMSCl (0.52 mL, 4.04 mmol, 1.6 equiv). The reaction mixture was stirred for 6 h at 45° C. The reaction mixture was allowed to cool to room temperature and was quenched with water (10 mL). The organic phase was separated, and the aqueous phase was extracted with EtOAc (2×20 mL). The combined organic phase was washed with brine (10 mL), dried over Na₂SO₄ and concentrated. The crude residue was purified by column chromatography (SiO₂, MeOH in dichloromethane, 0 to 20%) to afford the product.

Step f: To the product of step e (650 mg, 1.79 mmol, 1.0 equiv.) in dichloromethane (9 mL, 0.2 M) was added 2-methoxyethylamine (134 mg, 1.79 mmol, 1.0 equiv.) and Et₃N (0.63 mL, 3.58 mmol, 2.0 equiv.). After the mixture was stirred for 10 mins at 23° C., NaBH(OAc)₃ (760 mg, 3.58 mmol, 2.0 equiv.) was added. The reaction was stirred at 23° C. for 12 h, then quenched with sat. aq. NaHCO₃ solution (5 mL). The organic phase was separated, and the aqueous layer was extracted with EtOAc (2×10 mL). The combined organic phase was dried over Na₂SO₄, concentrated and the crude residue was purified by column chromatography (SiO₂, MeOH in dichloromethane, 0 to 10%) to afford the product of reductive amination.

Step g: A suspension of 1-(2-chloro-6-cyclopropylpyridin-4-yl)-5-methyl-2,8-dioxa-5-azonia-1-boranuidabicyclo[3.3.0]octane-3,7-dione (21.0 g, 68 mmol, 1.0 equiv., prepared according to example 18), 1-bromo-4-fluoro-2-nitrobenzene (15.7 g, 0.071 mol, 1.05 equiv.) and K₃PO₄ (43.3 g, 0.20 mol, 3.0 equiv.) in dioxane/water mixture (680 mL, 4:1 v/v, 0.1 M) was purged with N₂ for 10 minutes. Pd(dppf)Cl₂ (5.0 g, 7.0 mmol, 0.1 equiv.) was added, and the reaction was heated to 90° C. for 1 h under stirring. The resulting solution was cooled to room temperature, diluted with a ˜1:1 mixture of saturated aqueous NaCl and water (300 mL) and extracted with EtOAc (2×400 mL). The combined organics were dried over Na₂SO₄, filtered, and concentrated under vacuum. The crude residue was purified via column chromatography (SiO₂, 0-40% EtOAc gradient in hexanes) to afford the desired biaryl product.

Step h: To a cooled solution of the product from step g (16.4 g, 0.06 mol, 1.0 equiv.) in DMF (280 mL, 0.2 M), tetrahydroxydiboron (15 g, 0.17 mol, 3.0 equiv.) and 4,4-bipyridine (0.88 g, 5.60 mmol, 0.10 equiv.) were added at 0° C. The cooling bath was removed, and the mixture was stirred for 1 h at room temperature. Once full conversion was observed by LC/MS analysis the reaction mixture was quenched with water (500 mL), and the product was extracted with EtOAc (2×200 mL). The combined organic extract was dried over Na₂SO₄, filtered, and concentrated under reduced pressure. The crude product was purified by reversed phase column chromatography (SiO₂ C18, 10-100% CH₃CN gradient in water with 0.1% formic acid) to afford the corresponding aniline product.

Step i: To a solution of the product from step h (14.1 g, 0.054 mol, 1.0 equiv.) in CH₃CN (180 mL, 0.3 M), t-BuONO (8.53 mL, 0.065 mmol, 1.2 equiv.) and TMSN₃ (8.51 mL, 0.065 mol, 1.2 equiv.) were sequentially added at 0° C. The cooling bath was removed, and the resulting mixture was stirred at room temperature for 2 h. Once TLC analysis indicated a complete transformation the mixture was concentrated to dryness under reduced pressure. The crude aryl azide was used directly for the next step.

Step j: To a solution of the product from step i (250 mg, 0.87 mmol, 1.0 equiv.) and prop-1-yne (350 mg, 8.70 mmol, 10.0 equiv.) in degassed toluene (8.70 mL, 0.1 M) was added Cp*Ru(COD)Cl (69 mg, 0.087 mmol, 0.1 equiv.). The reaction vial was sealed and heated to 110° C. overnight. The resulting solution was cooled to room temperature and concentrated to dryness. The crude product was purified via column chromatography (SiO₂, 0-40% EtOAc gradient in hexanes) to afford two separate 1,2,3-triazole regioisomers, namely 2-chloro-6-cyclopropyl-4-[4-fluoro-2-(4-methyltriazol-1-yl)phenyl]pyridine (first eluting regioisomer) 2-chloro-6-cyclopropyl-4-[4-fluoro-2-(5-methyltriazol-1-yl)phenyl]pyridine (second eluting regioisomer).

Step k: A mixture of the product from step f (55 mg, 0.13 mmol, 1.0 equiv.), methyltriazole from step j (57 mg, 0.17 mmol, 1.3 equiv., first eluting regioisomer) and K₂CO₃ (54 mg, 0.39 mmol, 3.0 equiv.) in dioxane (2.60 ml) was degassed with a stream of nitrogen for ten minutes. CuI (25 mg, 0.13 mmol, 1.0 equiv.) and DMEDA (26 μL, 0.26 mmol, 2.0 equiv.) were added, and the reaction was heated for 16 h at 100° C. The resulting mixture was cooled to room temperature, diluted with aq. NH₄Cl (10 mL) and extracted with EtOAc (2×10 mL). The combined organic phase was washed with water (2×10 mL) and brine (10 mL), dried over Na₂SO₄, and concentrated to dryness. The crude residue was purified by reversed-phase column chromatography (SiO₂ C18, 10-100% CH₃CN in water with 0.1% formic acid) to afford corresponding coupling product. The residual material was then treated with trifluoroacetic acid/CH₂Cl₂ (v/v 1:10, 2 mL) at room temperature for 3 h. The mixture was concentrated under vacuum. The dry residue was then treated with 7M NH₃ in methanol (2 mL) for 30 min followed by solvent evaporation. The crude product was purified by reversed-phase column chromatography (SiO₂ C18, 10-100% CH₃CN in water with 0.1% formic acid) to afford the title compound. ¹H NMR (400 MHz, CDCl₃) δ 7.93 (t, J=1.6 Hz, 1H), 7.65 (dd, J=8.7, 5.7 Hz, 1H), 7.57 (d, J=1.4 Hz, 1H), 7.45 (d, J=0.9 Hz, 1H), 7.44-7.33 (m, 1H), 7.30-7.21 (m, 2H), 6.54 (d, J=1.3 Hz, 1H), 6.34 (d, J=1.6 Hz, 1H), 3.99 (s, 2H), 3.61-3.44 (m, 2H), 3.34 (s, 3H), 2.81 (t, J=5.0 Hz, 2H), 1.91 (s, 4H), 1.25 (t, J=7.1 Hz, 1H), 1.06-0.79 (m, 4H). ESI MS [M+H]⁺ for C₂₈H₂₉F₄N₇O₂ calcd 572.2, found 572.2.

### Example 131: 6-[6-cyclopropyl-4-[4-fluoro-2-(4-methyltriazol-1-yl)phenyl]pyridin-2-yl]-2-[(2-methoxyethylamino)methyl]-4-(trifluoromethyl)-1H-pyrrolo[2,3-c]pyridin-7-one

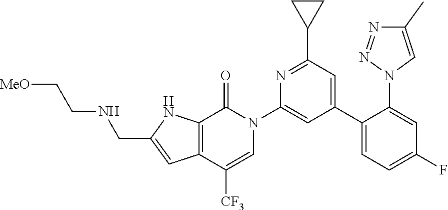 [SMILES: COCCNCc1cc2c(C(F)(F)F)cn(-c3cc(-c4ccc(F)cc4-n4cc(C)nn4)cc(C4CC4)n3)c(=O)c2[nH]1]

The title compound was prepared in a similar fashion to that described for example 130 starting from 2-chloro-6-cyclopropyl-4-[4-fluoro-2-(4-methyltriazol-1-yl)phenyl]pyridine (prepared according to example 130, step j, second eluting regioisomer). ¹H NMR (400 MHz, CDCl₃) δ 7.92 (q, J=1.4 Hz, 1H), 7.59-7.51 (m, 2H), 7.41-7.33 (m, 1H), 7.32-7.27 (m, 2H), 6.60 (d, J=1.4 Hz, 1H), 6.34 (d, J=1.8 Hz, 1H), 3.96 (d, J=7.7 Hz, 2H), 3.44 (q, J=5.2 Hz, 2H), 3.31 (d, J=7.7 Hz, 3H), 2.77 (q, J=4.9 Hz, 2H), 2.31 (d, J=0.8 Hz, 3H), 1.98-1.87 (m, 1H), 1.07-0.84 (m, 4H). ESI MS [M+H]⁺ for C₂₉H₂₇F₄N₇O₂ calcd 582.2, found 582.2.

### Example 132: 2-[2-(6-cyclopropyl-2-{2-[(3-methoxy-1-azetidinyl)methyl]-7-oxo-1,6-dihydro-1,4,6-triaza-6-indenyl}-4-pyridyl)-5-fluorophenyl]-1-methyl-4-imidazolecarbonitrile

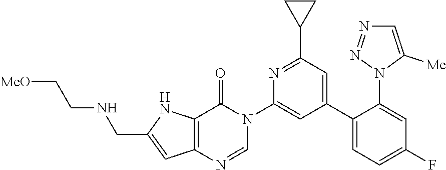 [SMILES: COCCNCc1cc2ncn(-c3cc(-c4ccc(F)cc4-n4nncc4C)cc(C4CC4)n3)c(=O)c2[nH]1]

The title compound was prepared in a similar fashion to example 130 using 6-[(2-methoxyethylamino)methyl]-5-(2-trimethylsilylethoxymethyl)-3H-pyrrolo[3,2-d]pyrimidin-4-one (prepared in an analogous fashion to that described in Example 22 using methoxyethanamine in step d). ¹H NMR (400 MHz, CDCl₃) δ 8.30 (s, 1H), 7.65 (dd, J=8.7, 5.7 Hz, 1H), 7.46 (dd, J=5.2, 1.2 Hz, 2H), 7.43-7.37 (m, 1H), 7.31-7.25 (m, 1H), 6.62 (d, J=1.4 Hz, 1H), 6.40 (s, 1H), 4.09 (s, 2H), 3.58-3.51 (m, 2H), 3.36 (s, 3H), 2.94-2.86 (m, 2H), 1.97-1.85 (m, 4H), 1.01-0.86 (m, 4H). ESI MS [M+H]⁺ C₂₇H₂₇FN₈O₂, calcd 515.2, found 515.2.

### Example 133: 2-cyclopropyl-5-{6-cyclopropyl-4-[4-fluoro-2-(5-methyl-1H-1,2,3-triazol-1-yl)phenyl]-2-pyridyl}-7-(trifluoromethyl)-3,5-dihydro-1,3,5-triaza-4-indenone

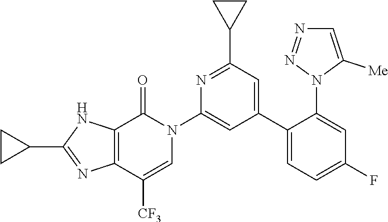 [SMILES: Cc1cnnn1-c1cc(F)ccc1-c1cc(C2CC2)nc(-n2cc(C(F)(F)F)c3nc(C4CC4)[nH]c3c2=O)c1]

The title compound was prepared in a similar fashion to that described for example 39 using 2-chloro-6-cyclopropyl-4-[4-fluoro-2-(5-methyltriazol-1-yl)phenyl]pyridine for step f (prepared according to example 130). ¹H NMR (400 MHz, CDCl₃) δ 12.06 (s, 1H), 7.94 (s, 1H), 7.65 (dd, J=8.7, 5.6 Hz, 1H), 7.47 (s, 1H), 7.45-7.39 (m, 1H), 7.39-7.35 (m, 1H), 7.28 (d, J=2.6 Hz, 2H), 6.74-6.67 (m, 1H), 2.05 (s, 2H), 1.75 (s, 2H), 1.00 (td, J=7.0, 6.5, 2.6 Hz, 4H), 0.92 (t, J=3.6 Hz, 2H), 0.84-0.76 (m, 2H). ESI MS [M+H]⁺ for C₂₇H₂₂F₄N₇O, calcd 536.2, found 536.2.

### Example 134: 6-[6-cyclopropyl-4-[2-[4-(difluoromethyl)-1,2,4-triazol-3-yl]-4-fluorophenyl]pyridin-2-yl]-7-oxo-4-(trifluoromethyl)-1H-pyrrolo[2,3-c]pyridine-3-carbonitrile

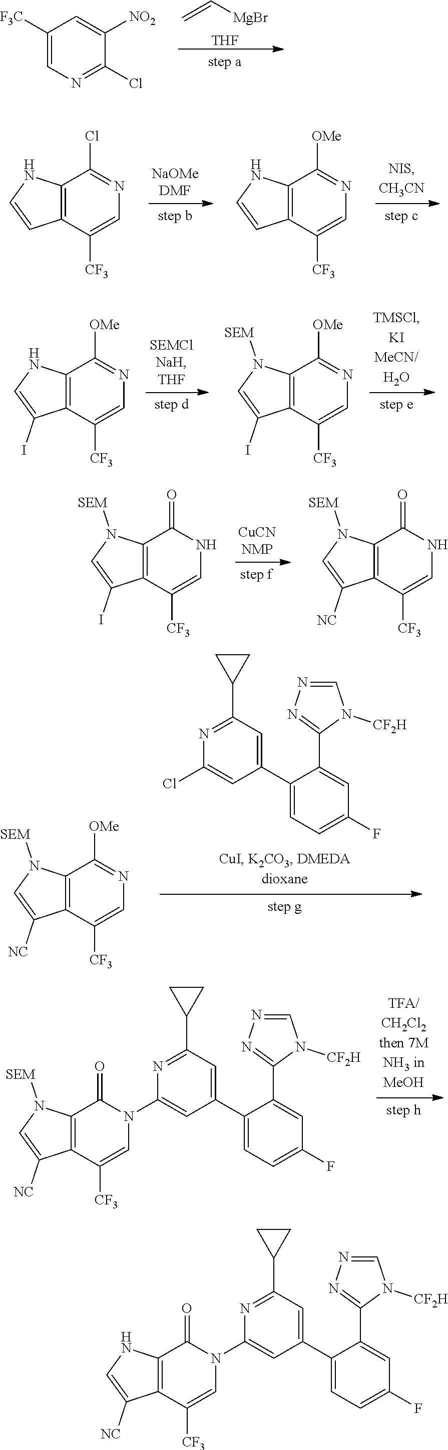

Step a: To a solution of 2-chloro-3-nitro-5-(trifluoromethyl)pyridine (5.3 g, 23 mmol, 1.0 equiv.) in THE (50 mL) was added vinylmagnesium bromide (1M in THF, 76 mL, 76 mmol, 3.3 equiv.) at −78° C. over 30 min. The resulting mixture was stirred at this temperature for another 30 min before being quenched with saturated aqueous NH₄Cl solution (20 mL). The product was extracted with EtOAc (2×80 mL). The combined organic phase was washed with brine, dried over Na₂SO₄ and concentrated. The crude residue was then purified by column chromatography (SiO₂, EtOAc in hexanes, 0 to 25%) to give the bicyclic product.

Step b: To a solution of the product from step a (1.02 g, 4.46 mmol, 1.0 equiv.) in DMF (22 mL) was added NaOMe (2.70 g, 44.6 mmol, 10 equiv.). The resulting mixture was heated at 130° C. for 1 h. After cooling back to room temperature, the mixture was diluted with EtOAc (70 mL) and washed sequentially with water (2×60 mL) and brine (60 mL). The combined organic extract was dried over Na₂SO₄ and concentrated. The crude product was purified by column chromatography (SiO₂, EtOAc in hexanes, 0 to 20%) to afford the product of nucleophilic substitution.

Step c: To a solution of the product from step b (1.50 g, 6.94 mmol, 1.0 equiv.) in MeCN (28 mL) was added N-iodosuccinimide (2.0 g, 9.0 mmol, 1.30 equiv.). The resulting mixture was stirred for 1 h when LCMS showed the completion of the iodination. The reaction was diluted with EtOAc (20 mL) and sequentially washed with aqueous saturated Na₂S₂O₃ (20 mL), water (20 mL) and brine (10 mL). The organic extract was concentrated under reduced pressure, and the crude product was purified by column chromatography (SiO₂, EtOAc in hexanes, 0 to 60%) to yield the desired product.

Step d: To a solution of the product from step c (500 mg, 1.46 mmol, 1.0 equiv.) in THE (7.30 mL, 0.2 M) was added NaH (120 mg, 60% in mineral oil, 2.92 mmol, 2.0 equiv.) at 0° C. SEM-Cl (370 mg, 2.19 mmol, 1.5 equiv.) was added after 15 min, and the reaction was stirred for 12 h at room temperature. The resulting solution was quenched with water (10 mL), and the aqueous layer was extracted with EtOAc (2×10 mL). The combined organic phase was dried over Na₂SO₄, concentrated and the crude residue was purified by column chromatography (SiO₂, EtOAc in hexanes, 10 to 50%) to afford SEM-protected bicyclic product.

Step e: The product from step d (670 mg, 1.42 mmol, 1.0 equiv.) was dissolved in a mixture of MeCN (7.10 mL, 0.2 M) and H₂O (77 μL, 4.26 mmol, 3.0 equiv). KI (380 mg, 2.27 mmol, 1.6 equiv.) and TMSCl (0.29 mL, 2.27 mmol, 1.6 equiv) were added sequentially, and the reaction mixture was stirred for 1 h at 45° C. Upon cooling to ambient temperature the mixture was diluted with EtOAc (20 mL) and quenched with water (5 mL). The organic phase was separated, and the aqueous phase was extracted with EtOAc (2×10 mL), the combined organic phase was then washed with brine (10 mL), dried over Na₂SO₄ and concentrated. The crude residue was purified by normal phase column chromatography (SiO₂, EtOAc in Hexanes, 0 to 60%), followed by additional purification by reversed-phase column chromatography (C18, 10-100% CH₃CN in water with 0.1% formic acid) to afford the corresponding product.

Step f: The product from step e (120 mg, 0.26 mmol, 1.0 equiv.) was dissolved in NMP (0.64 mL, 0.4 M) followed by addition of CuCN (48 mg, 0.52 mmol, 2.0 equiv). The reaction mixture was degassed by applying vacuum followed by backfilling with N₂. The reaction mixture was stirred for 3 h at 120° C. After cooling to room temperature the resulting mixture was diluted with EtOAc (10 mL) and washed with sat. ammonium chloride (4 mL). The organic phase was separated, washed with water (2×5 mL) dried over Na₂SO₄ and concentrated. The crude residue was purified by column chromatography (SiO₂, EtOAc in Hexanes, 0 to 30%), to afford the cyanation product.

Step g: A solution of the product from step f (35 mg, 0.098 mmol, 1.0 equiv.), 2-chloro-6-cyclopropyl-4-[2-[4-(difluoromethyl)-1,2,4-triazol-3-yl]-4-fluorophenyl]pyridine (48 mg, 0.13 mmol, 1.3 equiv. prepared according to example 68 starting from methyl 2-bromo-5-fluorobenzoate on step a) and K₂CO₃ (41 mg, 0.29 mmol, 3.0 equiv.) in dioxane (2.0 ml) was degassed with a stream of bubbling nitrogen for ten minutes. CuI (19 mg, 0.098 mmol, 1.0 equiv.) and DMEDA (19 μL, 0.20 mmol, 2.0 equiv.) were added, and the reaction was heated for 16 h at 100° C. The reaction was cooled to room temperature, diluted with EtOAc (10 mL) and quenched diluted with aq. NH₄Cl (5 mL). The organic phase was separated, and the aqueous phase was additionally extracted with EtOAc (2×5 mL). The combined organic phase was washed with water (2×5 mL) and brine (5 mL), dried over Na₂SO₄, and concentrated to dryness. The crude residue was purified by reversed-phase column chromatography (C18 SiO₂, 10-100% CH₃CN in water with 0.1% formic acid) to afford the desired coupling product.

Step h: The product from the step g (50 mg, 0.072 mmol, 1.0 equiv.) was treated with trifluoroacetic acid/dichloromethane (v/v 1:1, 1 mL) at room temperature for 3 h. The mixture was concentrated to dryness under vacuum. The dry residue was treated with 7M NH₃ in methanol (1 mL) for 30 min followed by solvent evaporation. The crude residue was purified by reversed-phase column chromatography (C18 SiO₂, 10-100% CH₃CN in water with 0.1% formic acid) to afford the title compound. ¹H NMR (400 MHz, CDCl₃) δ 8.84 (s, 1H), 8.16-8.05 (m, 1H), 8.01 (d, J=9.8 Hz, 2H), 7.77 (d, J=8.0 Hz, 1H), 7.67 (s, 1H), 7.42 (s, 1H), 7.19 (s, 1H), 6.87 (t, J=59.2 Hz, 1H), 2.09 (td, J=8.3, 4.3 Hz, 1H), 1.08 (dd, J=23.7, 6.5 Hz, 4H). ESI MS [M+H]⁺ for C₂₆H₁₅F₆N₇O calcd 556.1, found 556.1.

### Example 135: 6-[6-cyclopropyl-4-[4-fluoro-2-(4-methyl-1,2,4-triazol-3-yl)phenyl]pyridin-2-yl]-7-oxo-4-(trifluoromethyl)-1H-pyrrolo[2,3-c]pyridine-3-carbonitrile

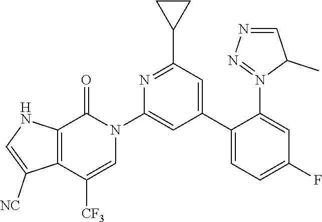

The title compound was prepared in a similar fashion to that described for example 134 from 7-oxo-4-(trifluoromethyl)-1-(2-trimethylsilylethoxymethyl)-6H-pyrrolo[2,3-c]pyridine-3-carbonitrile and 2-chloro-6-cyclopropyl-4-[4-fluoro-2-(4-methyl-1,2,4-triazol-3-yl)phenyl]pyridine (prepared according to example 28). ¹H NMR (400 MHz, CDCl₃) δ 8.22 (s, 1H), 8.09 (s, 1H), 7.59 (dd, J=8.6, 5.3 Hz, 1H), 7.51 (d, J=9.8 Hz, 1H), 7.46-7.32 (m, 2H), 7.15 (d, J=1.4 Hz, 1H), 6.99 (d, J=1.4 Hz, 1H), 3.17 (s, 3H), 1.99 (ddd, J=12.8, 8.0, 4.8 Hz, 1H), 1.08-0.91 (m, 4H). ESI MS [M+H]⁺ for C₂₆H₁₇F₄N₇O calcd 520.1, found 520.1.

### Example 136: 6-{6-cyclopropyl-4-[4-fluoro-2-(1-methyl-2-imidazolyl)phenyl]-2-pyridyl}-7-oxo-4-(trifluoromethyl)-1,6-dihydro-1,6-diaza-3-indenecarbonitrile

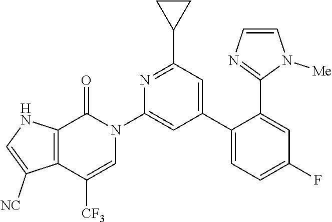 [SMILES: Cn1ccnc1-c1cc(F)ccc1-c1cc(C2CC2)nc(-n2cc(C(F)(F)F)c3c(C#N)c[nH]c3c2=O)c1]

The title compound was prepared in a similar fashion to that described for example 134 from 7-oxo-4-(trifluoromethyl)-1-(2-trimethylsilylethoxymethyl)-6H-pyrrolo[2,3-c]pyridine-3-carbonitrile and 2-chloro-6-cyclopropyl-4-[4-fluoro-2-(1-methylimidazol-2-yl)phenyl]pyridine (prepared according to example 51). ¹H NMR (400 MHz, DMSO-d₆) δ 8.27 (s, 1H), 8.00 (s, 1H), 7.71 (dd, J=8.7, 5.7 Hz, 1H), 7.54 (td, J=8.6, 2.8 Hz, 1H), 7.46 (dd, J=9.3, 2.7 Hz, 1H), 7.34 (d, J=1.4 Hz, 1H), 7.15 (d, J=1.2 Hz, 1H), 6.95 (d, J=1.1 Hz, 1H), 6.76 (d, J=1.4 Hz, 1H), 3.17 (s, 3H), 2.02 (tt, J=8.5, 4.7 Hz, 1H), 1.01-0.91 (m, 2H), 0.83-0.75 (m, 2H). ESI MS [M+H]⁺ for C₂₇H₁₈F₄N₆O, calcd 519.2, found 519.1.

### Example 137: 6-[6-cyclopropyl-4-[4-fluoro-2-(4-methyl-1,2,4-triazol-3-yl)phenyl]pyridin-2-yl]-2-(hydroxymethyl)-4-(trifluoromethyl)-1H-pyrrolo[2,3-c]pyridin-7-one

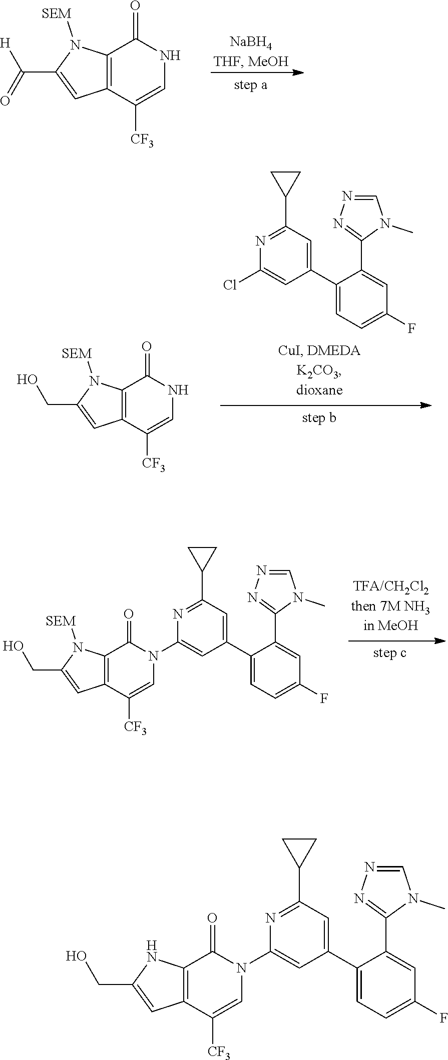

Step a: To a solution of 7-oxo-4-(trifluoromethyl)-1-(2-trimethylsilylethoxymethyl)-6H-pyrrolo[2,3-c]pyridine-2-carbaldehyde (3.9 g, 8.28 mmol, 1.0 equiv., prepared according to example 130) in THF (40 mL, 0.2 M) and MeOH (40 mL, 0.2 M) was added NaBH₄ (630 mg, 16.6 mmol, 2.0 equiv.) at 0° C. The resulting mixture was stirred at 0° C. for 1 h, then diluted with EtOAc (30 mL) and carefully quenched with 1M HCl (10 mL) at 0° C. The organic layer was separated, washed with brine (10 mL), dried over Na₂SO₄ and concentrated under reduced pressure. The crude carbinol product was used directly for the next step.

Step b: A solution of the product from step a (300 mg, 0.83 mmol, 1.0 equiv.), 2-chloro-6-cyclopropyl-4-[4-fluoro-2-(4-methyl-1,2,4-triazol-3-yl)phenyl]pyridine (380 mg, 1.08 mmol, 1.3 equiv. prepared according to example 28) and K₂CO₃ (340 mg, 2.50 mmol, 3.0 equiv.) in dioxane (17 ml, 0.05 M) was degassed with a stream of bubbling nitrogen for 10 minutes. CuI (160 mg, 0.83 mmol, 1.0 equiv.) and DMEDA (0.16 mL, 1.66 mmol, 2.0 equiv.) were added, and the reaction was heated for 16 h at 100° C. The resulting suspension was cooled to room temperature, diluted with aq. sat. NH₄Cl (10 mL) and extracted with EtOAc (2×10 mL). The combined organic extract was washed with water (2×10 mL) and brine (10 mL), dried over Na₂SO₄, and concentrated to dryness. The crude residue was purified by reversed-phase column chromatography (C18 SiO₂, 10-100% CH₃CN in water with 0.1% formic acid) to afford desired coupling product.

Step c: The product from the step b (200 mg, 0.30 mmol, 1.0 equiv.) was treated with trifluoroacetic acid/dichloromethane mixture (v/v 1:1, 3 mL) at 0° C. for 30 min. The resulting solution was concentrated under vacuum, then treated with 7M NH₃ in methanol (3.0 mL) for 30 min followed by concentration under vacuum. The crude product was purified by reversed-phase column chromatography (C18 SiO₂, 10-100% CH₃CN in water with 0.1% formic acid) to afford the title compound. ¹H NMR (400 MHz, CD₃OD) δ 8.48 (s, 1H), 7.88 (q, J=1.5 Hz, 1H), 7.83 (dd, J=8.7, 5.4 Hz, 1H), 7.55 (td, J=8.4, 2.7 Hz, 1H), 7.48 (dd, J=8.8, 2.7 Hz, 1H), 7.39 (d, J=1.4 Hz, 1H), 7.05 (d, J=1.5 Hz, 1H), 6.45 (d, J=1.6 Hz, 1H), 4.75 (s, 2H), 3.32 (s, 3H), 2.09 (tt, J=8.0, 4.8 Hz, 1H), 1.10-0.88 (m, 4H). ESI MS [M+H]⁺ for C₂₆H₂₀F₄N₆O₂ calcd 525.2, found 525.2.

### Example 138: 3-[6-cyclopropyl-4-[4-fluoro-2-[5-(fluoromethyl)triazol-1-yl]phenyl]pyridin-2-yl]-6-[(2-methoxyethylamino)methyl]-5H-pyrrolo[3,2-d]pyrimidin-4-one

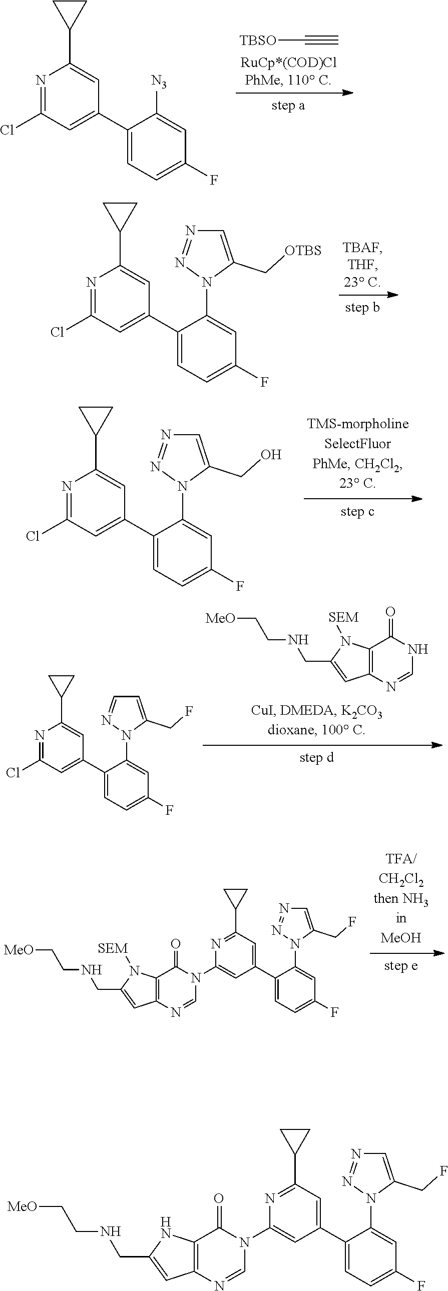

Step a: To a solution of 4-(2-azido-4-fluorophenyl)-2-chloro-6-cyclopropylpyridine (7.16 g, 24.8 mmol, 1.0 equiv. prepared according to example 130) and t-butyldimethylsilyl propargyl ether (6.34 g, 37.2 mmol, 1.50 equiv.) in toluene (124 mL, 0.20 M) was added RuCp*(COD)Cl (0.94 g, 2.48 mmol, 0.1 equiv.). The reaction mixture was degassed by 3 cycles of vacuum/nitrogen refill and heated to 110° C. for 16 h. Once the reaction was cooled to room temperature, the solvent was evaporated to dryness under reduced pressure. The crude product was purified via column chromatography (SiO₂, 0 to 40% EtOAc/hexanes) to afford the desired 1,2,3-triazole product.

Step b: The product from step a (8.0 g, 17.4 mmol, 1.0 equiv.) was dissolved in THF (174 mL, 0.1 M) and TBAF (1.0 M in THF, 23 mL, 1.30 equiv.) was added, and the mixture was stirred for 1 h. The resulting solution was quenched with water (50 mL) and extracted with EtOAc (2×50 mL). The combined organic extract was dried over Na₂SO₄ and concentrated under reduced pressure. The crude residue was fractionated by column chromatography (SiO₂, EtOAc in Hexane, 0 to 80%) to afford the corresponding hydroxymethyl-1,2,3-triazole.

Step c: TMS-morpholine (7.10 mL, 41.2 mmol, 7.10 equiv.) was added to Deoxo-Fluor® (50% wt solution in PhMe, 17.9 g, 40.6 mmol, 7.0 eq) at 0° C. The ice bath was subsequently removed, and the mixture was warmed to room temperature and stirred for 1 h, during which time it became increasingly heterogeneous, and a white precipitate formed. The solution of the product from step b (2.0 g, 5.80 mmol, 1.0 equiv.) in dichloromethane (29 mL) was added dropwise over 1 min at 0° C. The reaction was warmed to room temperature, and stirred for 45 min. Upon completion (TLC monitoring) the reaction was quenched with sat. aq. NaHCO₃ (10 mL) and diluted with dichloromethane (20 mL). The organic layer was separated, and the aqueous layer was extracted with dichloromethane (2×20 mL). The combined organic layers were dried over Na₂SO₄ and concentrated to dryness. The crude residue was purified via silica gel flash column chromatography (0 to 80% EtOAc/hexanes) to afford the fluorinated product.

Step d: A solution of 6-[(2-methoxyethylamino)methyl]-5-(2-trimethylsilylethoxy-methyl)-3H-pyrrolo[3,2-d]pyrimidin-4-one (20 mg, 0.057 mmol, 1.0 equiv., prepared according to example 22 using 2-methoxyethylamine in step d), product from step c (26 mg, 0.074 mmol, 1.3 equiv.) and K₂CO₃ (24 mg, 0.17 mmol, 3.0 equiv.) in dioxane (1.10 ml, 0.05 M) was degassed with a stream of bubbling nitrogen for ten minutes. CuI (11 mg, 0.057 mmol, 1.0 equiv.) and DMEDA (11 μL, 0.11 mmol, 2.0 equiv.) were added, and the reaction was heated for 16 h at 100° C. The reaction was cooled to room temperature, diluted with aq. NH₄Cl (3 mL), and extracted with EtOAc (2×5 mL). The combined organic phase was washed with water (2×5 mL) and brine (5 mL), dried over Na₂SO₄, and concentrated to dryness. The crude residue was purified by reversed-phase column chromatography (SiO₂ C18, 10-100% CH₃CN in water with 0.1% formic acid) to afford desired coupling product.

Step e: The product from the step d (20 mg, 0.030 mmol, 1.0 equiv.) was treated with trifluoroacetic acid/dichloromethane (v/v 1:1, 1 mL) at room temperature for 3 h. The resulting solution was concentrated to dryness under reduced pressure. The crude residue was dissolved in 7M NH₃ solution in methanol (1 mL), and the obtained solution was stirred for 30 min followed by solvent evaporation. The crude product was purified by reversed-phase column chromatography (SiO₂ C18, 10-100% CH₃CN in water with 0.1% formic acid) to afford the title compound. ¹H NMR (400 MHz, CDCl₃) δ 8.26 (s, 1H), 7.81 (d, J=3.2 Hz, 1H), 7.66 (dd, J=8.7, 5.7 Hz, 1H), 7.44 (td, J=8.1, 2.6 Hz, 1H), 7.37 (d, J=1.4 Hz, 1H), 7.31 (dd, J=8.1, 2.6 Hz, 1H), 6.74 (s, 1H), 6.36 (s, 1H), 5.19 (s, 1H), 5.07 (s, 1H), 4.04 (s, 2H), 3.54 (t, J=4.9 Hz, 2H), 3.38 (s, 3H), 2.88 (s, 2H), 1.92 (tt, J=7.9, 4.9 Hz, 1H), 1.25 (s, 2H), 1.09-0.75 (m, 4H). ESI MS [M+H]⁺ for C₂₇H₂₆F₂N₈O₂ calcd 533.2, found 533.2.

### Example 139: 4-[2-(2-{[(S)-2-methoxypropylamino]methyl}-7-oxo-1,6-dihydro-1,4,6-triaza-6-indenyl)-6-cyclopropyl-4-pyridyl]-3-[5-(fluoromethyl)-1H-1,2,3-triazol-1-yl]benzonitrile

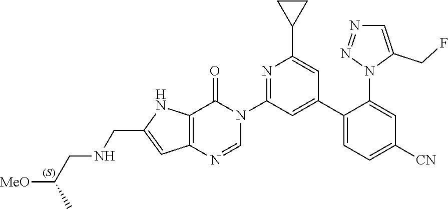

The title compound was prepared in a similar fashion to that described for example 138 using 6-[[[(2S)-2-methoxypropyl]amino]methyl]-3,5-dihydropyrrolo[3,2-d]pyrimidin-4-one (prepared according to example 22 using (2S)-2-methoxypropan-1-amine for reductive amination step). ¹H NMR (400 MHz, CDCl₃) δ 8.30 (s, 1H), 7.99 (dd, J=8.1, 1.7 Hz, 1H), 7.88-7.78 (m, 3H), 7.45 (d, J=1.4 Hz, 1H), 6.78 (d, J=1.4 Hz, 1H), 6.37 (s, 1H), 5.27-5.06 (m, 2H), 4.10-3.94 (m, 2H), 3.57-3.47 (m, 1H), 3.36 (s, 3H), 2.79-2.56 (m, 3H), 1.97-1.87 (m, 1H), 1.14 (d, J=6.1 Hz, 3H), 1.05-0.92 (m, 4H); C₂₉H₂₈FN₉O₂, calcd 554.2, found 554.2.

### Example 140: 4-cyclopropyl-6-[6-cyclopropyl-4-[4-fluoro-2-(5-methylpyrazol-1-yl)phenyl]pyridin-2-yl]-2-[[(1-methylcyclobutyl)amino]methyl]-1H-pyrrolo[2,3-c]pyridin-7-one

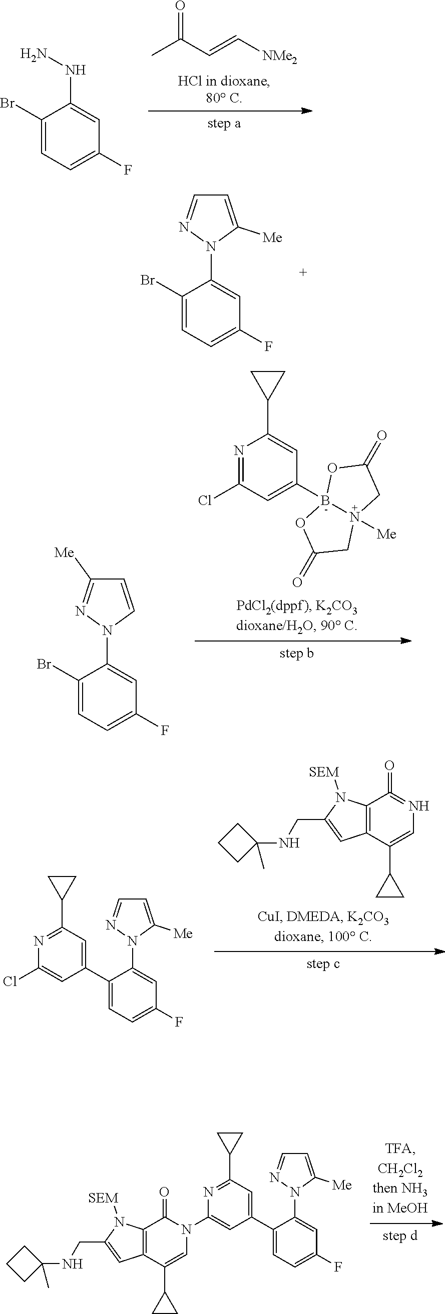

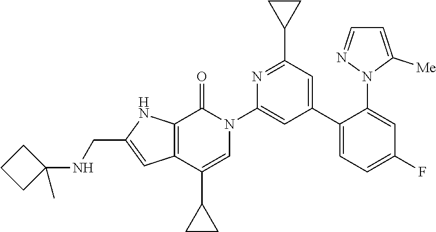 [SMILES: Cc1ccnn1-c1cc(F)ccc1-c1cc(C2CC2)nc(-n2cc(C3CC3)c3cc(CNC4(C)CCC4)[nH]c3c2=O)c1]

Step a: To the solution of (2-bromo-5-fluorophenyl)hydrazinehydrochloride (200 mg, 0.97 mmol, 1.0 equiv.), 4-(dimethylamino)-3-buten-2-one (120 mg, 1.02 mmol, 1.05 equiv.) was added, followed by 4N HCl in dioxane (5 mL, 0.20 M). The reaction was then heated to 80° C. for 10 mins, then cooled to room temperature. The reaction mixture was neutralized with sat. aq. sodium bicarbonate to pH ˜7 and extracted with EtOAc (2×5 mL). The combined organics were dried over Na₂SO₄, filtered, and concentrated under vacuum. The crude residue was purified via silica gel flash column chromatography (0 to 20% EtOAc/hexanes) to afford two corresponding pyrazole regioisomers, namely 2-chloro-6-cyclopropyl-4-[4-fluoro-2-(3-methylpyrazol-1-yl)phenyl]pyridine (first eluting regioisomer) and 2-chloro-6-cyclopropyl-4-[4-fluoro-2-(5-methylpyrazol-1-yl)phenyl]pyridine (second eluting regioisomer). Regioisomers were assigned by TR-FRET.

Step b: A solution of the 1-(2-chloro-6-cyclopropylpyridin-4-yl)-5-methyl-2,8-dioxa-5-azonia-1-boranuidabicyclo[3.3.0]octane-3,7-dione (2.40 g, 7.64 mmol, 1.0 equiv., prepared according to example 18), the product from step a (1.50 g, 5.89 mmol, 1.10 equiv., second eluting regioisomer), K₂CO₃ (2.40 g, 17.7 mmol, 3.0 equiv.) in dioxane water mixture (v/v 4:1, 45 mL) was purged with N₂ for 10 minutes. Pd(dppf)Cl₂ (450 mg, 0.59 mmol, 0.1 equiv.) was added, and the reaction was heated to 90° C. and stirred for 16 hours. The resulting biphasic solution was cooled to room temperature, quenched with a ˜1:1 mixture of saturated aqueous NaCl and water (10 mL) and extracted with EtOAc (2×25 mL). The combined organics were dried over Na₂SO₄, filtered, and concentrated under vacuum. The crude product was then purified by reversed phase column chromatography (C18 SiO₂, 10-100% CH₃CN in water with 0.1% formic acid) to afford the corresponding triaryl coupling product.

Step c: A solution of 4-cyclopropyl-2-[[(1-methylcyclobutyl)amino]methyl]-1-(2-trimethylsilylethoxymethyl)-6H-pyrrolo[2,3-c]pyridin-7-one (46 mg, 0.11 mmol, 1.0 equiv., prepared according to example 1 using 1-methylcyclobutan-1-amine in step e), product from step b (50 mg, 0.15 mmol, 1.3 equiv.) and K₂CO₃ (46 mg, 0.33 mmol, 3.0 equiv.) in dioxane (2.20 ml, 0.05 M) was degassed with a stream of bubbling nitrogen for ten minutes. CuI (21 mg, 0.11 mmol, 1.0 equiv.) and DMEDA (22 μL, 0.22 mmol, 2.0 equiv.) were added, and the reaction was heated for 16 h at 100° C. Then the mixture was cooled to room temperature, diluted EtOAc (5 mL) and aqueous saturated NH₄Cl (3 mL). The organic phase was separated, and the aqueous phase was extracted with EtOAc (2×4 mL). The combined organic extract was washed with water (2×10 mL) and brine (5 mL), dried over Na₂SO₄, and concentrated to dryness under reduced pressure. The crude residue was purified by reversed phase column chromatography (C18 SiO₂, 10-100% CH₃CN in water with 0.1% formic acid) to yield the coupling product.

Step d: The product from the step c (20 mg, 0.029 mmol, 1.0 equiv.) was treated with trifluoroacetic acid/dichloromethane (1 mL, 1:1 v/v) at room temperature for 3 h. The mixture was concentrated under vacuum, then the residue was treated with 7M NH₃ in methanol (1 mL) for 30 min followed by solvent evaporation. The crude residue was then purified by reversed phase column chromatography (C18 SiO₂, 10-100% CH₃CN in water with 0.1% formic acid) to afford the title compound. ¹H NMR (400 MHz, CDCl₃) δ 7.68-7.59 (m, 2H), 7.58 (d, J=1.7 Hz, 1H), 7.31-7.18 (m, 4H), 6.38-6.27 (m, 2H), 6.06 (d, J=1.6 Hz, 1H), 3.85 (s, 2H), 1.99-1.86 (m, 3H), 1.84 (s, 3H), 1.82-1.65 (m, 3H), 1.33-1.19 (m, 5H), 0.98-0.78 (m, 7H), 0.71-0.62 (m, 2H). ESI MS [M+H]⁺ for C₃₄H₃₅FN₆O calcd 563.3, found 563.3.

### Example 141: 6-[6-cyclopropyl-4-[4-fluoro-2-(4-methyl-1,2,4-triazol-3-yl)phenyl]pyridin-2-yl]-4-(trifluoromethyl)-1H-pyrrolo[2,3-c]pyridin-7-one

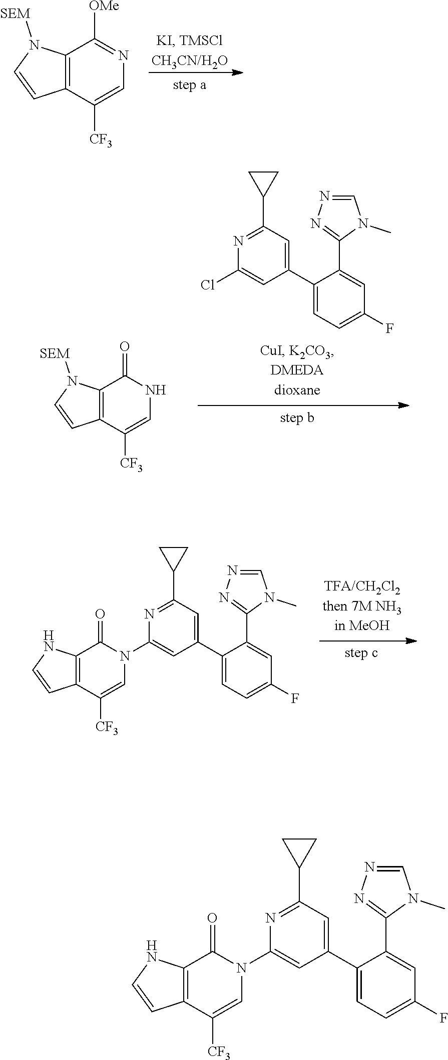 [SMILES: COc1ncc(C(F)(F)F)c2ccn(COCC[Si](C)(C)C)c12 ; "KI, TMSClCH3CN/H2O" ; Cn1cnnc1-c1cc(F)ccc1-c1cc(Cl)nc(C2CC2)c1 ; C[Si](C)(C)CCOCn1ccc2c(C(F)(F)F)c[nH]c(=O)c21 ; Cn1cnnc1-c1cc(F)ccc1-c1cc(C2CC2)nc(-n2cc(C(F)(F)F)c3cc[nH]c3c2=O)c1 ; Cn1cnnc1-c1cc(F)ccc1-c1cc(C2CC2)nc(-n2cc(C(F)(F)F)c3cc[nH]c3c2=O)c1]

Step a: 2-[[7-Methoxy-4-(trifluoromethyl)pyrrolo[2,3-c]pyridin-1-yl]methoxy]-ethyltrimethylsilane (120 mg, 0.35 mmol, 1.0 equiv., prepared according to example 130 step c) was dissolved in MeCN (1.80 mL, 0.2 M) and H₂O (19 μL, 4.26 mmol, 3.0 equiv). KI (93 mg, 0.56 mmol, 1.6 equiv.) and TMSCl (71 μL, 0.56 mmol, 1.6 equiv) were sequentially added, and the reaction mixture was stirred for 1 h at 45° C. Once complete consumption of the starting material was observed by TLC analysis the mixture cooled to room temperature, diluted with EtOAc (7 mL) and quenched with water (2 mL). The organic phase was separated, and the aqueous phase was extracted with EtOAc (2×5 mL). The combined organic extract was washed with brine (5 mL), dried over Na₂SO₄ and concentrated to dryness under reduced pressure. The crude residue was purified by column chromatography (SiO₂, EtOAc in Hexanes, 0 to 60%), to afford desired product.

Step b: A solution of product from step a (40 mg, 0.12 mmol, 1.0 equiv.), 2-chloro-6-cyclopropyl-4-[4-fluoro-2-(4-methyl-1,2,4-triazol-3-yl)phenyl]pyridine (51 mg, 0.16 mmol, 1.3 equiv., prepared according to example 28) and K₂CO₃ (50 mg, 0.36 mmol, 3.0 equiv.) in dioxane (2.40 ml, 0.05 M) was degassed with a stream of bubbling nitrogen for ten minutes. CuI (23 mg, 0.12 mmol, 1.0 equiv.) and DMEDA (24 μL, 0.24 mmol, 2.0 equiv.) were added, and the reaction was heated for 16 h at 100° C. The resulting mixture was cooled to room temperature, diluted with EtOAc (5 ml) and aq. NH₄Cl (3 mL). The organic phase was separated, and the aqueous phase was additionally extracted with EtOAc (2×3 mL). The combined organic extract was washed with water (2×5 mL) and brine (5 mL), dried over Na₂SO₄, and concentrated to dryness under reduced pressure. The crude residue was purified by reversed phase column chromatography (C18 SiO₂, 10-100% CH₃CN in water with 0.1% formic acid) to afford desired coupling product.

Step c: The product from the step b (60 mg, 0.099 mmol, 1.0 equiv.) was treated with trifluoroacetic acid/dichloromethane (1 mL, 1:1 v/v) at room temperature for 3 h. The mixture was concentrated under vacuum, and the residue was dissolved in 7M NH₃ in methanol (1 mL). After stirring for 30 min at room temperature the mixture was concentrated, and the crude product was directly purified by reversed-phase column chromatography (C18 SiO₂, 10-100% CH₃CN in water with 0.1% formic acid) to afford the title compound. ¹H NMR (400 MHz, CDCl₃) δ 10.20 (s, 1H), 8.21 (s, 1H), 7.91 (q, J=1.5 Hz, 1H), 7.60 (dd, J=8.6, 5.4 Hz, 1H), 7.46 (d, J=1.4 Hz, 1H), 7.44-7.33 (m, 2H), 7.32 (s, 1H), 6.92 (d, J=1.4 Hz, 1H), 6.55 (p, J=1.6 Hz, 1H), 3.14 (s, 3H), 2.07-1.92 (m, 1H), 1.08-0.90 (m, 4H). ESI MS [M+H]⁺ for C₂₅H₁₈F₄N₆O calcd 495.2, found 495.2.

### Example 142: 6-[6-cyclopropyl-4-[4-fluoro-2-[5-(trifluoromethyl)pyrazol-1-yl]phenyl]pyridin-2-yl]-2-[(2-methoxyethylamino)methyl]-4-(trifluoromethyl)-1H-pyrrolo[2,3-c]pyridin-7-one

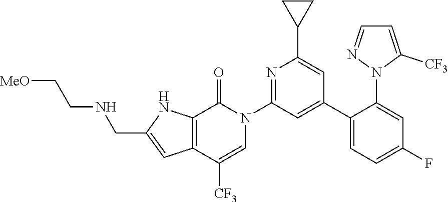 [SMILES: COCCNCc1cc2c(C(F)(F)F)cn(-c3cc(-c4ccc(F)cc4-n4nccc4C(F)(F)F)cc(C4CC4)n3)c(=O)c2[nH]1]

The title compound was prepared in a similar fashion to that described for example 130 from 2-[(2-methoxyethylamino)methyl]-4-(trifluoromethyl)-1-(2-trimethylsilylethoxy-methyl)-6H-pyrrolo[2,3-c]pyridin-7-one (84 mg, 0.20 mmol, 1.0 equiv.) and 2-chloro-6-cyclopropyl-4-[4-fluoro-2-[5-(trifluoromethyl)pyrazol-1-yl]phenyl]pyridine (100 mg, 0.26 mmol, 1.30 equiv., prepared according to example 103). ¹H NMR (400 MHz, CDCl₃) δ 7.86 (q, J=1.4 Hz, 1H), 7.67-7.60 (m, 2H), 7.51 (d, J=1.3 Hz, 1H), 7.36 (ddd, J=8.7, 7.7, 2.7 Hz, 1H), 7.23 (dd, J=8.4, 2.7 Hz, 2H), 6.74 (dd, J=1.9, 0.9 Hz, 1H), 6.70 (d, J=1.3 Hz, 1H), 6.34 (d, J=1.6 Hz, 1H), 4.01 (s, 2H), 3.51 (t, J=5.0 Hz, 2H), 3.36 (s, 3H), 2.83 (t, J=5.0 Hz, 2H), 1.94 (tt, J=8.1, 4.8 Hz, 1H), 1.04-0.83 (m, 4H). ESI MS [M+H]⁺ for C₃₀H₂₅F₇N₆O₂ calcd 635.2, found 635.2.

### Example 143: 2-[6-Cyclopropyl-4-[4-fluoro-2-(5-methylpyrimidin-4-yl)phenyl]pyridin-2-yl]-6-[[(3S)-3-methylpiperidin-1-yl]methyl]-3H-isoindol-1-one

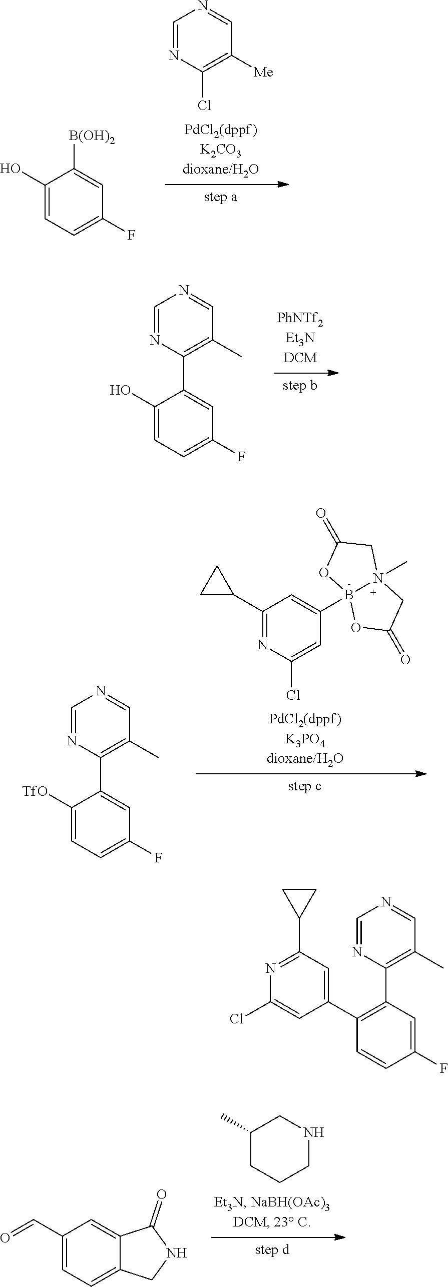

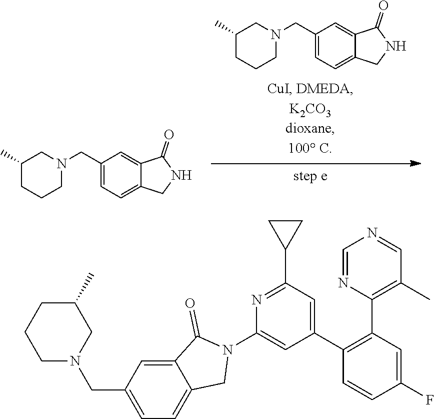 [SMILES: C[C@H]1CCCN(Cc2ccc3c(c2)C(=O)NC3)C1 ; C[C@H]1CCCN(Cc2ccc3c(c2)C(=O)NC3)C1 ; Cc1cncnc1-c1cc(F)ccc1-c1cc(C2CC2)nc(N2Cc3ccc(CN4CCC[C@H](C)C4)cc3C2=O)c1]

Step a: A mixture of (5-fluoro-2-hydroxyphenyl)boronic acid (450 mg, 2.9 mmol, 1 equiv.), 4-chloro-5-methylpyrimidine (371 mg, 2.9 mmol, 1 equiv.) and K₂CO₃ (1.2 g, 8.7 mmol, 3 equiv.) in dioxane/water mixture (9.9 mL, 10:1 v/v) was degassed by three cycles of vacuum/backfilling with nitrogen followed by the addition of PdCl₂(dppf) (211 mg, 0.3 mmol, 0.1 equiv.). The resulting mixture was heated at 90° C. overnight, then cooled to room temperature and concentrated to dryness under reduced pressure. The crude residue was fractionated by reversed phase column chromatography (C18 SiO₂, 0 to 100% CH₃CN in water with 0.1% formic acid) to afford 4-fluoro-2-(5-methylpyrimidin-4-yl)phenol.

Step b: Triethylamine (0.9 mL, 6.4 mmol, 3 equiv.) and PhNTf₂ (1.14 g, 3.2 mmol, 1.5 equiv.) were added sequentially to a suspension of the product from step a (436 mg, 2.1 mmol, 1 equiv.) in dichloromethane (10.7 ml, 0.2 M) at room temperature. The resulting mixture was stirred for 3 h at room temperature. Once TLC analysis indicated complete conversion of the starting material the mixture was concentrated to dryness, and the dry residue was directly purified by column chromatography (SiO₂, 0-100% EtOAc gradient in hexanes) to afford [4-fluoro-2-(5-methylpyrimidin-4-yl)phenyl] trifluoromethanesulfonate.

Step c: A mixture of the triflate product from step b (509 mg, 1.5 mmol, 1 equiv.) and 1-(2-chloro-6-cyclopropylpyridin-4-yl)-5-methyl-2,8-dioxa-5-azonia-1-boranuida-bicyclo[3.3.0]octane-3,7-dione (514 mg, 1.7 mmol, 1.1 equiv., prepared according to example 18) and K₃PO₄ (964 mg, 4.5 mmol, 3 equiv.) in dioxane/water mixture (15 mL, 4:1 v/v) was degassed by three cycles of vacuum/backfilling with nitrogen followed by the addition of PdCl₂(dppf) (44 mg, 0.06 mmol, 0.04 equiv.). The resulting mixture was heated at 90° C. for 1 h before cooling to room temperature and dilution with water (10 mL) and EtOAc (20 mL). The organic phase was separated, and the aqueous phase was additionally extracted with EtOAc (20 mL). The combined organic phase was dried over MgSO₄ and concentrated to dryness under reduced pressure. The crude residue was fractionated by reversed phase column chromatography (C18 SiO₂, 0 to 100% CH₃CN in water with 0.1% formic acid) to afford 4-[2-(2-chloro-6-cyclopropylpyridin-4-yl)-5-fluorophenyl]-5-methylpyrimidine.

Step d: To a solution of 3-oxo-1,2-dihydroisoindole-5-carbaldehyde (500 mg, 3.1 mmol, 1.0 equiv.) in dichloromethane (15.0 mL) was added (S)-3-methylpiperidine hydrochloride (463 mg, 3.4 mmol, 1.1 equiv.) and triethylamine (474 μL, 3.4 mmol, 1.1 equiv.). The reaction mixture was stirred for 5 minutes at room temperature and NaBH(OAc)₃ (986 mg, 4.6 mmol, 1.5 equiv.) was added. The reaction was stirred for an additional 16 hours at room temperature at which point it was quenched with saturated aqueous NaHCO₃ and extracted with dichloromethane. The combined organics were dried over MgSO₄, filtered, and concentrated under vacuum. The crude residue was purified by column chromatography (SiO₂, MeOH in dichloromethane, 0 to 20%) to afford the desired product.

Step e: This step was performed in a similar fashion to step d in example 73 to afford the title compound. ¹H NMR (400 MHz, CDCl₃) δ 9.08 (s, 1H), 8.44 (s, 1H), 8.23 (d, J=1.3 Hz, 1H), 7.77 (d, J=1.4 Hz, 1H), 7.28-7.20 (m, 1H), 7.56 (ddd, J=11.2, 6.8, 3.5 Hz, 2H), 7.44 (d, J=7.7 Hz, 1H), 7.11 (dd, J=8.8, 2.7 Hz, 1H), 6.53 (d, J=1.4 Hz, 1H), 4.94 (s, 2H), 3.55 (s, 2H), 2.83-2.71 (m, 2H), 2.02 (q, J=7.2, 5.3 Hz, 2H), 1.90-1.74 (m, 2H), 1.72-1.53 (m, 5H), 0.93-0.77 (m, 9H). ESI MS [M+H]⁺ for C₃₄H₃₅FN₅O, calcd 548.3, found 548.2.

### Example 144: 2-{6-Cyclopropyl-4-[4-fluoro-2-(4-methyl-4H-1,2,4-triazol-3-yl)phenyl]-2-pyridyl}-1-oxo-2,5,6,7,8,9-hexahydroβ-carboline-4-carbonitrile

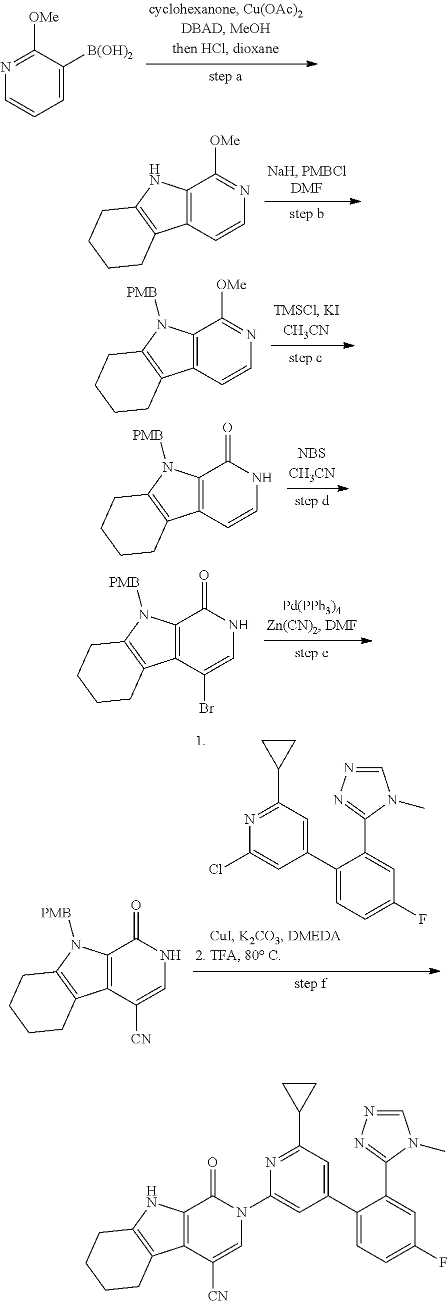

Step a: To a mixture of a (2-methoxypyridin-3-yl)boronic acid (2.2 g, 15.0 mmol) and di-tert-butylazodicarboxylate (2.3 g, 1.0 mmol) in MeOH (60 mL) was added Cu(OAc)₂ monohydrate (100 mg, 0.5 mmol), and the resulting mixture was stirred for 1 h at 65° C. and cooled to room temperature. Then hydrochloric acid solution in dioxane (4M, 20 mL) was added, and the reaction was stirred at 23° C. for 15 min. The reaction mixture was concentrated to dryness under reduced pressure. The crude residue was mixed with cyclohexanone (1.5 g, 15.0 mmol) and aq. H₂SO₄ solution (10 mL, 4 wt %). The resulting mixture was refluxed for 2 h, cooled to room temperature and concentrated to dryness under reduced pressure. The crude residue was partitioned between dichloromethane (50 mL) and aq. sat. NaHCO₃ (10 mL). The organic phase was separated, and the aqueous phase was additionally extracted with dichloromethane (2×40 mL). The combined organic extract was washed with water (50 mL), dried over Na₂SO₄, and the solvent was removed under reduced pressure. The crude residue was purified by column chromatography (SiO₂, 0-80% EtOAc gradient in hexanes) to afford the desired tetrahydroazacarbazole derivative.

Step b: To a solution of the product of step a (0.5 g, 2.47 mmol, 1.0 equiv.) in THE (12 mL) was added NaH (197.6 mg, 4.94 mmol, 2.0 equiv., 60 wt % in mineral oil) at 0° C. The reaction mixture was stirred at 0° C. for 10 min before 4-methoxybenzyl chloride (0.77 g, 4.94 mmol, 2.0 equiv.) was added. The mixture was stirred at 23° C. for 12 h, then quenched with sat. aq. NH₄Cl (5 mL) and diluted with EtOAc (20 mL). The organic phase was separated, and the aqueous layer was extracted with EtOAc (15 mL). The combined organic phase was dried over Na₂SO₄ and the solvent was removed under reduced pressure. The crude material was purified by column chromatography (SiO₂, 0-80% EtOAc gradient in hexanes) to afford the desired alkylation product.

Step c: The reaction was performed in a similar fashion to Example 22, step e.

Step d: To a solution of the product from step c (0.47 g, 1.54 mmol, 1.0 equiv.) in THE (8 mL) was added N-bromosuccinimide (0.27 g, 1.54 mmol, 1.0 equiv.). The resulting mixture was stirred for 3 h. Once complete transformation was observed by LCMS analysis the solution was concentrated to dryness under reduced pressure. The residue was partitioned between EtOAc (50 mL) and water (50 mL). The organic phase was separated, and the aqueous layer was extracted with EtOAc (30 mL). The combined organic phase was dried over Na₂SO₄, concentrated and the crude residue was purified by column chromatography (SiO₂, 0-80% EtOAc gradient in hexanes) to afford the bromination product.

Step e: To a solution of the product from step d (161 mg, 0.41 mmol, 1.0 equiv.) in DMF (2 mL) under N₂ was added Zn(CN)₂ (120 mg, 1.02 mmol, 2.5 equiv.) and Pd(PPh₃)₄ (96 mg, 0.08 mmol, 0.2 equiv). The resulting mixture was heated at 110° C. overnight. After cooling down to room temperature, the reaction mixture was diluted with EtOAc (20 mL), filtered through a Celite® pad, and then washed with water (30 mL) and brine (30 mL). The organic layer was separated, dried over Na₂SO₄ and concentrated to dryness under vacuum. The crude residue was purified by column chromatography (SiO₂, 0 to 10% MeOH gradient in dichloromethane) to afford the cyanation product.

Step f: To a mixture of product from step e (45.0 mg, 0.13 mmol, 1.0 equiv.) and 2-chloro-6-cyclopropyl-4-[4-fluoro-2-(4-methyl-1,2,4-triazol-3-yl)phenyl]pyridine (47.1 mg, 0.13 mmol, 1.0 equiv., prepared according to example 28) in dioxane (2.8 mL) were added CuI (25 mg, 0.13 mmol, 1.0 equiv.), N,N′-dimethylethylenediamine (23 mg, 0.26 mmol, 2.0 equiv.) and K₂CO₃ (53 mg, 0.38 mmol, 3.0 equiv.). The reaction mixture was degassed by purging N₂ for 5 minutes and heated at 110° C. for 12 hours under vigorous stirring. The reaction mixture was cooled to room temperature, diluted with EtOAc (10 mL) and aq. sat. NH₄Cl (5 mL). The organic phase was separated, washed with brine (5 mL), dried over Na₂SO₄, and concentrated to dryness under reduced pressure. The residual material was dissolved in TFA (4 mL) and stirred at 80° C. for 8 h. The mixture was cooled to 23° C. and concentrated under reduced pressure. The crude residue was then purified by prep-HPLC (C18 SiO₂, 10-90% CH₃CN in water with 0.1% formic acid) to afford the title compound. ¹H NMR (400 MHz, CDCl₃) δ 9.55 (s, 1H), 8.20 (s, 1H), 7.99 (s, 1H), 7.60 (dd, J=8.6, 5.4 Hz, 1H), 7.44-7.31 (m, 3H), 6.94 (d, J=1.2 Hz, 1H), 3.15 (s, 3H), 2.88 (s, 2H), 2.69 (s, 2H), 2.03-1.94 (m, 1H), 1.90-1.82 (m, 4H), 1.05-0.99 (m, 2H), 0.98-0.92 (m, 2H). ESI MS [M+H]⁺ C₂₉H₂₄FN₇O, calcd 506.2, found 506.2.

### BIOLOGICAL ASSAY EXAMPLES

The affinity with which compounds of the present disclosure bind to Cbl-b was assessed using probe displacement homologous time resolved fluorescence (HTRF) assays. The assays used a BODIPY™ conjugated probe (Example 54 from WO 2020264398) and biotinylated Cbl-b. The assays were performed in assay buffer consisting of 20 mM Hepes, 150 mM NaCl, 0.01% Triton X-100, 0.5 mM TCEP, 0.01% BSA. On the day of the assay, a 20 point, 1:2 master serial dilution of each compound was prepared in DMSO to span a final concentration range of 10 μM to 0 nM. Two hundred nanoliters of diluted compound was added to each well of a 384-well plate. Fifteen microliters of biotinylated—Cbl-b resuspended in assay buffer were added to each well and the plate incubated for 60 minutes at room temperature prior to addition of 5 μL of BODIPY™ probe in assay buffer. Final assay conditions included 0.4 nM of Cbl-b and 150 nM of BODIPY™ probe. After a further 15 minutes of incubation at room temperature, 5 μl of Streptavidin-Terbium cryptate reagent at 5-fold final concentration was added to each well of the 384-well plate. Binding of a probe to Cbl-b results in an increase of HTRF signal. HTRF signal was measured by Envision plate reader, while competition of a compound of the present disclosure with probe results in a decrease of signal. Percentage maximum activity in each test well was calculated based on DMSO (maximum activity, 0% displacement) and no protein control wells (baseline activity, 100% displacement). Binding affinity was determined from a dose response curve fitted using a standard four parameter fit equation. See Table 3 for data for compounds (Cbl-b Binding (IC₅₀)).

Certain compounds were also evaluated in an IL-2 secretion assay. On the day of the assay, a 16 point, 1:2 master serial dilution of each compound was prepared in Opti-MEM to span a final concentration range of 10 μM to 300 pM. Assays were setup in CORNING® tissue culture-treated 384-well microplates containing 60 nL of each dilution. Jurkat cells grown in RPMI-1640 supplemented with 10% FBS, 1% Glutamax, and 1% Pen/Strep were collected, resuspended in Opti-MEM. 50,000 cells/well and added to the compound plates. After a short spin (1200 rpm for 1 min), the plates were incubated at 37° C. for 1 hour. The cells were activated by adding 15 μL of IMUNOCULT™ Human CD3/CD28 T Cell Activator (STEMCELL Technologies) diluted in Opti-MEM. After 24 h of incubation at 37° C., aliquots of culture supernatants were transferred to OptiPlate-384 (PerkinElmer) microplates. The level of IL-2 secretion in the supernatants was then determined using the IL-2 (human) AlphaLISA Detection Kit (PerkinElmer) according to the manufacturer's recommendations. The AlphaLISA signal was measured using an EnVision plate reader (PerkinElmer). EC₅₀ values were determined by fitting the data to a standard 4-parameter logistic equation. See Table 3 for data for select compounds (IL-2 Secretion (EC₅₀)).

TABLE 3
Potency of select compounds.

| | Cbl-b Binding | IL-2 Secretion |
|---|---|---|
| Example # | (IC₅₀) | (EC₅₀) |
| 1 | +++ | +++ |
| 2 | +++ | +++ |
| 3 | ++ | + |
| 4 | + | − |
| 5 | +++ | +++ |
| 6 | +++ | +++ |
| 7 | ++ | + |
| 8 | +++ | +++ |
| 9 | +++ | +++ |
| 10 | +++ | n.d. |
| 11 | +++ | +++ |
| 12 | +++ | ++ |
| 13 | +++ | n.d. |
| 14 | +++ | +++ |
| 15 | +++ | +++ |
| 16 | +++ | +++ |
| 17 | +++ | ++ |
| 18 | +++ | + |
| 19 | +++ | +++ |
| 20 | +++ | ++ |
| 21 | +++ | ++ |
| 22 | +++ | +++ |
| 23 | + | − |
| 24 | ++ | + |
| 25 | +++ | +++ |
| 26 | +++ | + |
| 27 | ++ | − |
| 28 | +++ | +++ |
| 29 | +++ | +++ |
| 30 | +++ | ++ |
| 31 | +++ | +++ |
| 32 | +++ | ++ |
| 33 | +++ | ++ |
| 34 | +++ | +++ |
| 35 | +++ | ++ |
| 36 | +++ | ++ |
| 37 | +++ | +++ |
| 38 | +++ | ++ |
| 39 | +++ | +++ |
| 40 | +++ | +++ |
| 41 | +++ | +++ |
| 42 | +++ | +++ |
| 43 | +++ | +++ |
| 44 | +++ | ++ |
| 45 | +++ | ++ |
| 46 | +++ | +++ |
| 47 | +++ | +++ |
| 48 | +++ | +++ |
| 49 | +++ | n.d. |
| 50 | +++ | +++ |
| 51 | +++ | +++ |
| 52 | +++ | +++ |
| 53 | +++ | +++ |
| 54 | +++ | +++ |
| 55 | +++ | +++ |
| 56 | +++ | ++ |
| 57 | +++ | +++ |
| 58 | +++ | +++ |
| 59 | +++ | +++ |
| 60 | +++ | ++ |
| 61 | +++ | ++ |
| 62 | +++ | +++ |
| 63 | +++ | ++ |
| 64 | +++ | ++ |
| 65 | +++ | +++ |
| 66 | +++ | ++ |
| 67 | +++ | ++ |
| 68 | +++ | +++ |
| 69 | +++ | +++ |
| 70 | +++ | +++ |
| 71 | +++ | +++ |
| 72 | +++ | n.d. |
| 73 | +++ | ++ |
| 74 | +++ | ++ |
| 75 | +++ | +++ |
| 76 | +++ | +++ |
| 77 | +++ | +++ |
| 78 | +++ | n.d. |
| 79 | +++ | n.d. |
| 80 | +++ | n.d. |
| 81 | +++ | +++ |
| 82 | +++ | +++ |
| 83 | +++ | +++ |
| 84 | +++ | +++ |
| 85 | +++ | +++ |
| 86 | +++ | +++ |
| 87 | +++ | +++ |
| 88 | +++ | +++ |
| 89 | +++ | +++ |
| 90 | +++ | ++ |
| 91 | +++ | +++ |
| 92 | +++ | +++ |
| 93 | +++ | +++ |
| 94 | +++ | ++ |
| 95 | +++ | ++ |
| 96 | +++ | +++ |
| 97 | +++ | ++ |
| 98 | +++ | +++ |
| 99 | +++ | +++ |
| 100 | +++ | +++ |
| 101 | +++ | +++ |
| 102 | +++ | +++ |
| 103 | +++ | ++ |
| 104 | +++ | ++ |
| 105 | +++ | ++ |
| 106 | +++ | +++ |
| 107 | +++ | +++ |
| 108 | +++ | +++ |
| 109 | +++ | ++ |
| 110 | +++ | +++ |
| 111 | +++ | +++ |
| 112 | +++ | +++ |
| 113 | +++ | n.d. |
| 114 (major + minor) | +++ | +++ |
| 115 | +++ | +++ |
| 116 | +++ | +++ |
| 117 | +++ | ++ |
| 118 | +++ | ++ |
| 119 | +++ | ++ |
| 120 | +++ | +++ |
| 121 | +++ | ++ |
| 122 | +++ | ++ |
| 123 | +++ | ++ |
| 124 | +++ | ++ |
| 125 | +++ | ++ |
| 126 | +++ | +++ |
| 127 | +++ | +++ |
| 128 | +++ | ++ |
| 129 (Regio-1 + Regio-2) | +++ | +++ |
| 130 | +++ | +++ |
| 131 | +++ | +++ |
| 132 | +++ | +++ |
| 133 | +++ | ++ |
| 134 | +++ | +++ |
| 135 | +++ | +++ |
| 136 | +++ | +++ |
| 137 | +++ | +++ |
| 138 | +++ | +++ |
| 139 | +++ | +++ |
| 140 | +++ | ++ |
| 141 | +++ | +++ |
| 142 | +++ | ++ |
| 143 | +++ | + |
| 144 | +++ | +++ |

Less than 100 nM (+++),
100 nM to 1 μM (++),
greater than 1 μM to 5 μM (+),
>5 μM (−),
n.d. = not determined

Particular embodiments of this disclosure are described herein, including the best mode known to the inventors for carrying out the disclosure. Upon reading the foregoing, description, variations of the disclosed embodiments may become apparent to individuals working in the art, and it is expected that those skilled artisans may employ such variations as appropriate. Accordingly, it is intended that the disclosure be practiced otherwise than as specifically described herein, and that the disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All publications, patent applications, accession numbers, and other references cited in this specification are herein incorporated by reference for the purpose described herein.

1. A compound having a structure according to Formula I: 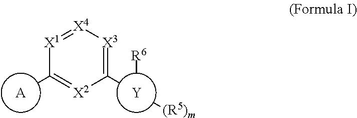
or a pharmaceutically acceptable salt thereof, wherein:
A has a formula selected from the group consisting of: 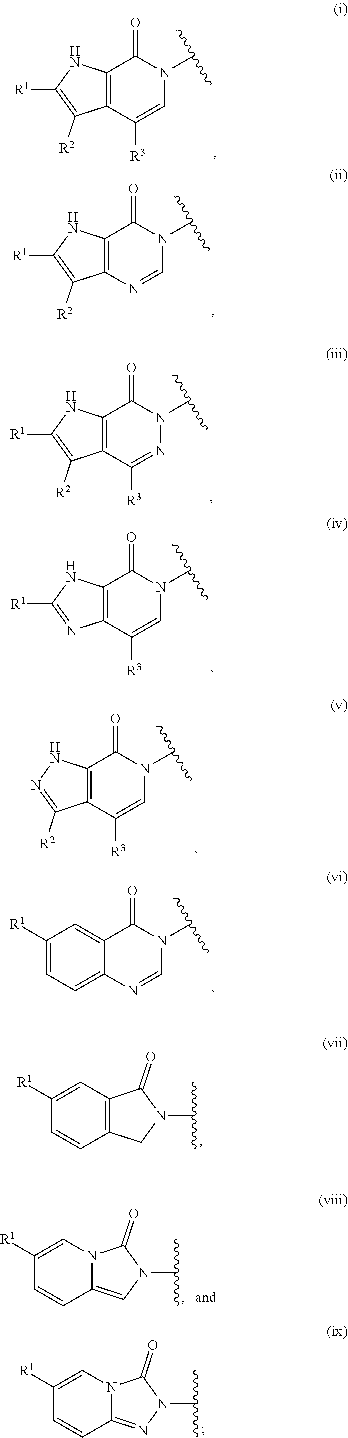
wherein:
R¹ is selected from the group consisting of —H, —C₁-C₆ alkyl, —C₁-C₆ haloalkyl, —C₁-C₆ hydroxyalkyl, -(Q¹)-O—(C₁-C₃ alkyl), —C(O)NH₂, —C(O)—(C₁-C₆-alkyl), -(Q¹)-NR^{1a}R^{1b}, -(Q¹)-(C₃-C₇ cycloalkyl), -(Q¹)-(5- to 6-membered heteroaryl), and -(Q¹)-(4- to 8-membered heterocycloalkyl); wherein said 5- to 6-membered heteroaryl has 1-3 ring heteroatoms independently selected from N, O, and S; said 4- to 8-membered heterocycloalkyl has 1-3 ring heteroatom or heteroatom groups independently selected from N, O, S, and S(O)₂; said C₁-C₃ alkyl is unsubstituted or substituted with —C₁-C₃ alkoxy; and said C₃-C₇ cycloalkyl, 4- to 8-membered heterocycloalkyl, and 5- to 6-membered heteroaryl are unsubstituted or substituted with 1-2 substituents independently selected from halo, —OH, —C₁-C₃ alkyl, —C₁-C₃ alkoxy, and —C(O)(C₁-C₃ alkyl);
Q¹ is absent, unsubstituted —(C₁-C₃ alkylene)-, or —(C₁-C₃ alkylene)- substituted with 1-3 R^{q};
each R^{q} is independently halo, —OH, or —NH₂;
R^{1a} and R^{1b} are independently selected from the group consisting of —H, —C₁-C₆ alkyl, —C₁-C₆ haloalkyl, phenyl, —(C₁-C₃ alkylene)-O—(C₁-C₃ alkyl), —C₃-C₆ cycloalkyl, —(C₁-C₃ alkylene)-(C₃-C₆ cycloalkyl), —S(O)₂(C₁-C₆ alkyl), 5- to 6-membered heteroaryl having 1-3 ring heteroatoms independently selected from N, O, and S, and 4- to 8-membered heterocycloalkyl having 1-3 ring heteroatom or heteroatom groups independently selected from N, O, S, and S(O)₂; wherein said phenyl, —(C₁-C₃ alkylene)-O—(C₁-C₃ alkyl), —C₃-C₆ cycloalkyl, —(C₁-C₃ alkylene)-(C₃-C₆ cycloalkyl), 5- to 6-membered heteroaryl, and 4- to 8-membered heterocycloalkyl are unsubstituted or substituted with 1-3 R^{1c};
each R^{1c}, when present, is independently halo, —OH, —C₁-C₃ alkyl, —C₁-C₃ hydroxyalkyl, or —C₁-C₃ haloalkyl;
R², when present, is —H, halo, —CN, —C₁-C₃ alkyl, —C₁-C₃ haloalkyl, —C₃-C₄ cycloalkyl, —S(O)₂(C₁-C₃ alkyl), —C(O)—NR^{2a}R^{2b}, or 5- to 6-membered heteroaryl having 1-3 ring heteroatoms independently selected from N, O, and S; or
R¹ and R² taken together with the atoms to which they are attached form —C₃-C₆ cycloalkyl;
R^{2a} and R^{2b} are independently —H, or —C₁-C₃ alkyl;
R³, when present, is —H, —CN, halo, —C₁-C₆ alkyl, —C₁-C₆ haloalkyl, —C₁-C₆ hydroxyalkyl, —C₂-C₃ alkenyl, —C₃-C₄ cycloalkyl, —S(O)₂(C₁-C₆ alkyl), —C(O)OH, or 5- to 6-membered heteroaryl having 1 to 4 ring heteroatoms independently selected from N, S, and O; and the 5- to 6-membered heteroaryl is unsubstituted or substituted with 1-3 substituents independently selected from —C₁-C₃ alkyl;
X¹, X² and X³ are each independently N or CH;
X⁴ is N or CR⁴;
R⁴, when present, is —H, halo, —CN, —OH, —C₁-C₆ alkyl, —C₁-C₆ haloalkyl, —C₁-C₆ alkoxy, —NR^{4a}R^{4b} or —C₃-C₈ cycloalkyl;
R^{4a} and R^{4b} are independently —H, —C₁-C₃ alkyl, or —(C₁-C₃ alkylene)-NR^{4c}R^{4d};
R^{4c} and R^{4d} are independently —H, or —C₁-C₃ alkyl;
Y is phenyl, or 5- to 6-membered heteroaryl having 1-3 ring heteroatoms independently selected from N, O, and S;
m is 0, 1, 2, or 3;
each R⁵ when present, is independently halo, —CN, —C₁-C₆ alkyl, —C₁-C₆ haloalkyl, or —C₁-C₆ alkoxy;
when ring A has a structure of formula (i), (ii), (iii), (iv), (v), or (vi), R⁶ is a 5- to 6-membered heteroaryl having 1 to 4 ring heteroatoms independently selected from N, O, and S, wherein R⁶ is unsubstituted or substituted with 1-3 R^{6a}; or
when ring A has a structure of formula (vii), (viii), or (ix), R⁶ is a 6-membered heteroaryl having 1 to 4 ring heteroatoms independently selected from N, O, and S, wherein R⁶ is unsubstituted or substituted with 1-3 R^{6a}; and
each R^{6a} is independently selected from the group consisting of —CN, —C₁-C₃ alkyl, —C₁-C₃ haloalkyl, and —C₁-C₃ hydroxyalkyl.

2. (canceled)

3. The compound of claim 1, or a pharmaceutically acceptable salt thereof, wherein:
R¹ is selected from the group consisting of —H, —C₁-C₆ alkyl, —C₁-C₆ haloalkyl, —C₁-C₆ hydroxyalkyl, -(Q¹)-O—(C₁-C₃ alkyl), -(Q¹)-NR^{1a}R^{1b}, -(Q¹)-(C₃-C₇ cycloalkyl), -(Q¹)-(5- to 6-membered heteroaryl), and -(Q¹)-(4- to 8-membered heterocycloalkyl); wherein said 5- to 6-membered heteroaryl has 1-3 ring heteroatoms independently selected from N, O, and S; said 4- to 8-membered heterocycloalkyl has 1-3 ring heteroatom or heteroatom groups independently selected from N, O, S, and S(O)₂; said C₁-C₃ alkyl is unsubstituted or substituted with —C₁-C₃ alkoxy; and said C₃-C₇ cycloalkyl, 4- to 8-membered heterocycloalkyl, and 5- to 6-membered heteroaryl are unsubstituted or substituted with 1-2 substituents independently selected from halo, —OH, —C₁-C₃ alkyl, —C₁-C₃ alkoxy, and —C(O)(C₁-C₃ alkyl);
Q¹ is absent, unsubstituted —(C₁-C₃ alkylene)- or —(C₁-C₃ alkylene)- substituted with 1-3 R^{q};
each R^{q} is independently halo, —OH, or —NH₂;
R^{1a} and R^{1b} are independently selected from the group consisting of —H, —C₁-C₆ alkyl, —C₁-C₆ haloalkyl, —(C₁-C₃ alkylene)-O—(C₁-C₃ alkyl), —C₃-C₆ cycloalkyl, —(C₁-C₃ alkylene)-(C₃-C₆ cycloalkyl), and 4- to 8-membered heterocycloalkyl having 1-3 ring heteroatoms independently selected from N, O, and S; wherein said —(C₁-C₃ alkylene)-O—(C₁-C₃ alkyl), —C₃-C₆ cycloalkyl, —(C₁-C₃ alkylene)-(C₃-C₆ cycloalkyl), and 4- to 8-membered heterocycloalkyl are unsubstituted or substituted with 1-3 R^{1c}; and
each R^{1c}, when present, is independently halo, —OH, —C₁-C₃ alkyl, or —C₁-C₃ hydroxyalkyl.

4. (canceled)

5. The compound of claim 1, or a pharmaceutically acceptable salt thereof, wherein:
R¹ is —C₁-C₆ hydroxyalkyl, -(Q¹)-NR^{1a}R^{1b} or -(Q¹)-(4- to 8-membered heterocycloalkyl) having 1-3 ring heteroatom or heteroatom groups independently selected from N, O, S, and S(O)₂; and said 4- to 8-membered heterocycloalkyl is unsubstituted or substituted with 1-2 substituents independently selected from halo, —OH, —C₁-C₃ alkyl, and —C₁-C₃ alkoxy.

6. (canceled)

7. (canceled)

8. The compound of claim 1, or a pharmaceutically acceptable salt thereof, wherein R², when present is —H or —CN.

9. The compound of claim 1, or a pharmaceutically acceptable salt thereof, wherein R¹ and R² taken together with the atoms to which they are attached form —C₅-C₆ cycloalkyl.

10. The compound of claim 1, or a pharmaceutically acceptable salt thereof, wherein R³, when present, is —CN, —C₁-C₆ alkyl, —C₁-C₆ haloalkyl, —C₃-C₄ cycloalkyl, or —S(O)₂(C₁-C₆ alkyl).

11. (canceled)

12. (canceled)

13. The compound of claim 1, or a pharmaceutically acceptable salt thereof, having a structure of Formula Ia:

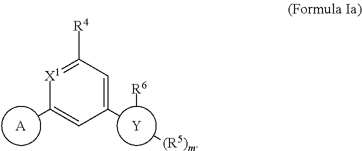

14.-16. (canceled)

17. The compound of claim 1, or a pharmaceutically acceptable salt thereof, wherein R⁴ is —H, —CN, —F, —Cl, —CH₃, —CF₃, —OH, —OCH₃, —OCH₂CH₃,

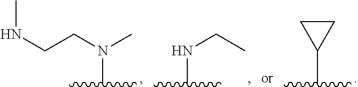

18. The compound of claim 1, or a pharmaceutically acceptable salt thereof, wherein Y is phenyl, or a 5-membered heteroaryl having 1-3 ring heteroatoms independently selected from N, O, and S.

19. The compound of claim 18, or a pharmaceutically acceptable salt thereof, wherein Y is phenyl or pyrazole.

20. (canceled)

21. The compound of claim 19, or a pharmaceutically acceptable salt thereof, wherein the compound has a structure of Formula Ib-1: 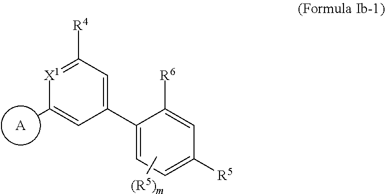
wherein m is 0 or 1.

22. (canceled)

23. (canceled)

24. The compound of claim 1, or a pharmaceutically acceptable salt thereof, wherein each R⁵, when present, is independently, —F, —CN, —CH₃, —CF₃, or —OCH₃.

25. (canceled)

26. (canceled)

27. The compound of claim 1, or a pharmaceutically acceptable salt thereof, wherein R⁶ is triazole, imidazole, pyrazole, oxazole, pyridine, pyridazine, or pyrimidine, each of which is unsubstituted, or substituted with 1-2 R^{6a}.

28.-32. (canceled)

33. The compound of claim 1, or a pharmaceutically acceptable salt thereof, wherein R^{6a} is —C₁-C₃ alkyl, or —C₁-C₃ haloalkyl.

34. The compound of claim 1, or a pharmaceutically acceptable salt thereof, wherein A is:

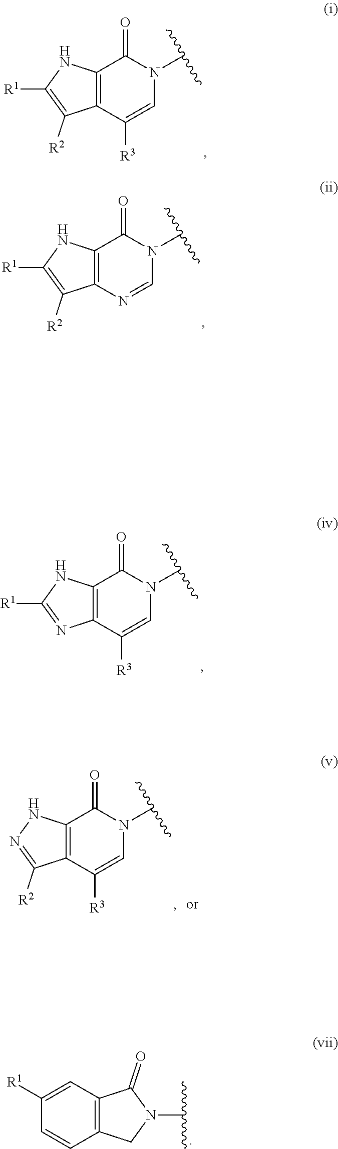

35. (canceled)

36. (canceled)

37. The compound of claim 1, or a pharmaceutically acceptable salt thereof, wherein A is

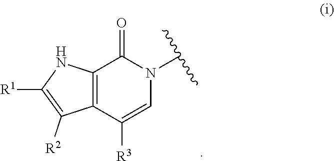

38. A compound selected from the group consisting of:

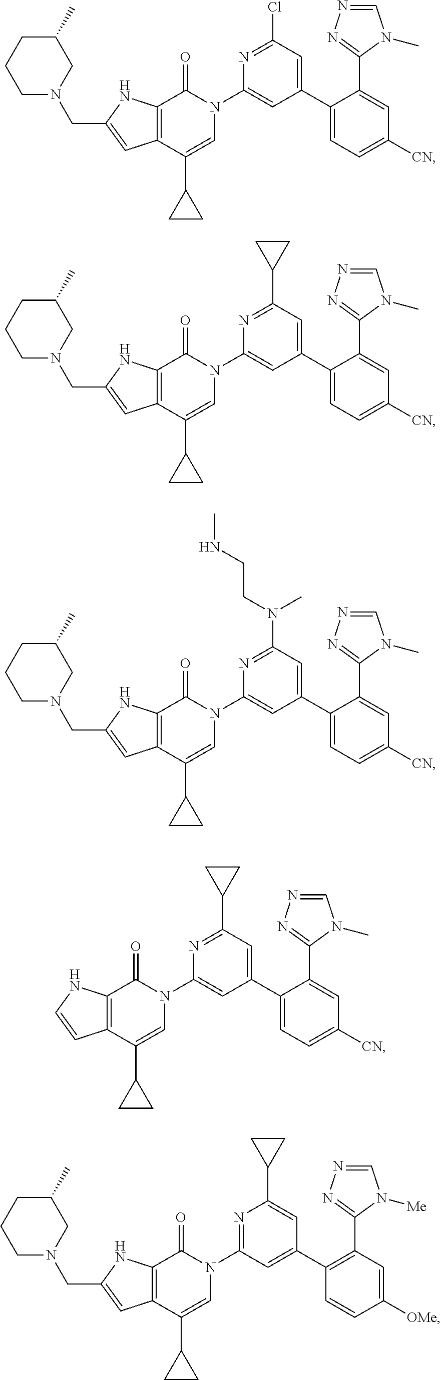

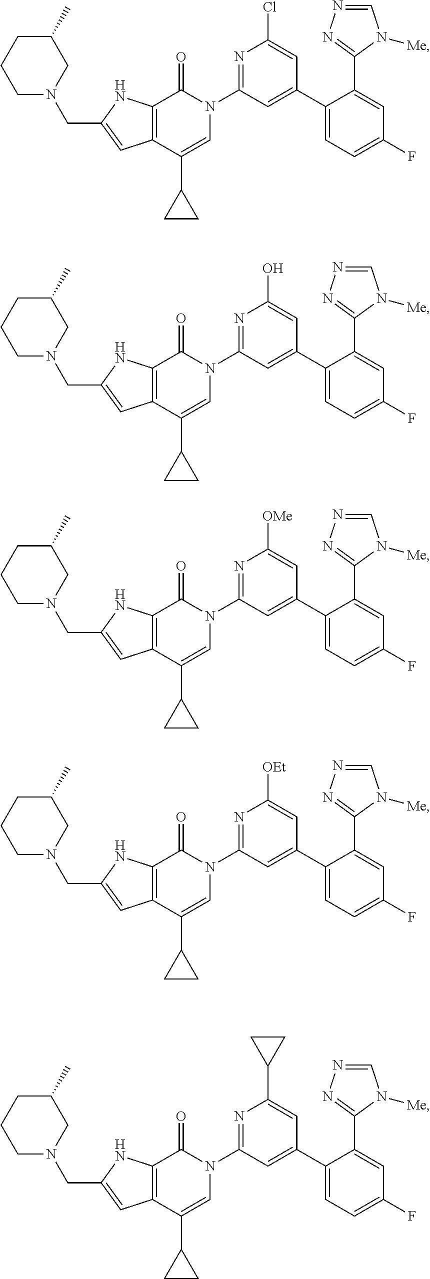 [SMILES: C[C@H]1CCCN(Cc2cc3c(C4CC4)cn(-c4cc(-c5ccc(F)cc5-c5nncn5C)cc(Cl)n4)c(=O)c3[nH]2)C1 ; C[C@H]1CCCN(Cc2cc3c(C4CC4)cn(-c4cc(-c5ccc(F)cc5-c5nncn5C)cc(O)n4)c(=O)c3[nH]2)C1 ; COc1cc(-c2ccc(F)cc2-c2nncn2C)cc(-n2cc(C3CC3)c3cc(CN4CCC[C@H](C)C4)[nH]c3c2=O)n1 ; CCOc1cc(-c2ccc(F)cc2-c2nncn2C)cc(-n2cc(C3CC3)c3cc(CN4CCC[C@H](C)C4)[nH]c3c2=O)n1 ; C[C@H]1CCCN(Cc2cc3c(C4CC4)cn(-c4cc(-c5ccc(F)cc5-c5nncn5C)cc(C5CC5)n4)c(=O)c3[nH]2)C1]

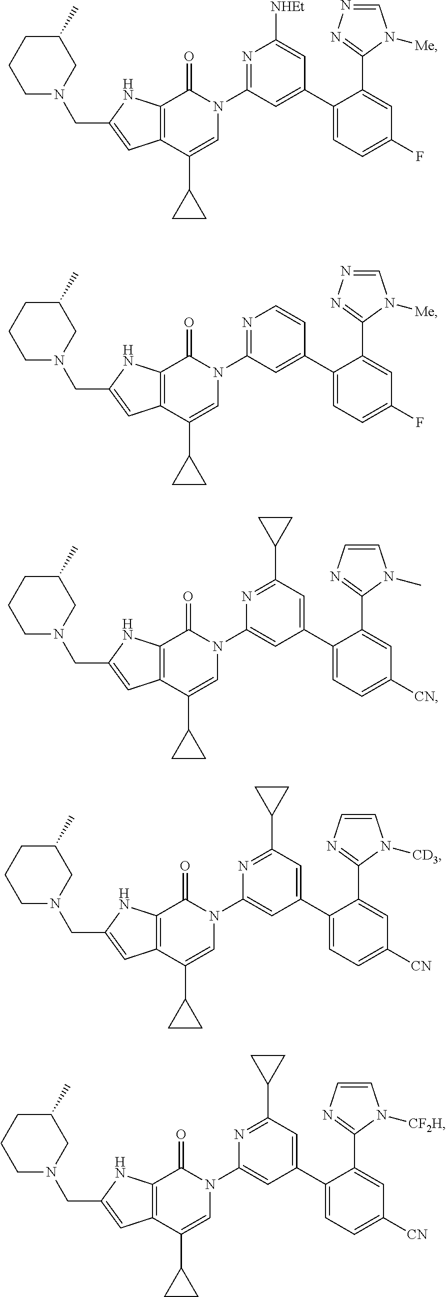

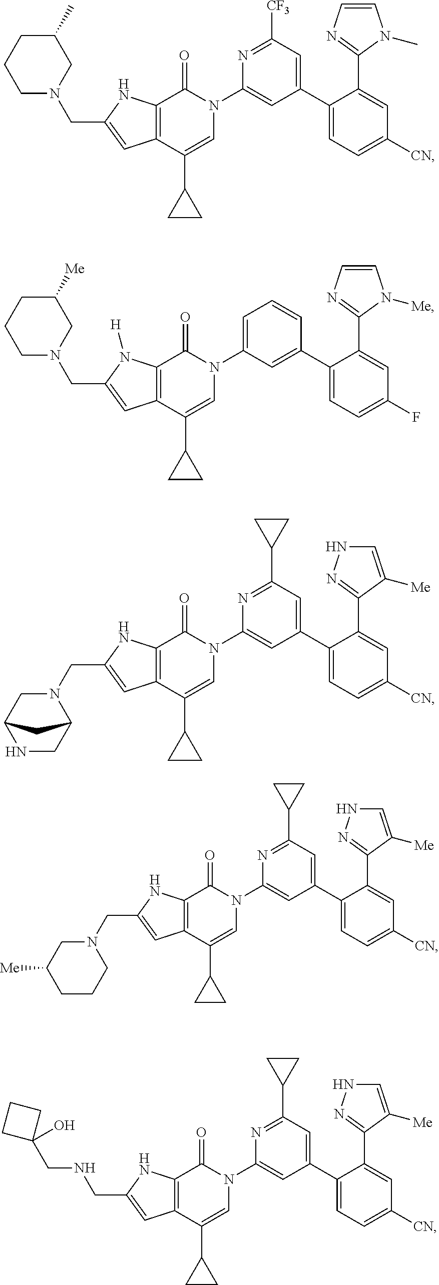 [SMILES: C[C@H]1CCCN(Cc2cc3c(C4CC4)cn(-c4cc(-c5ccc(C#N)cc5-c5nccn5C)cc(C(F)(F)F)n4)c(=O)c3[nH]2)C1 ; C[C@H]1CCCN(Cc2cc3c(C4CC4)cn(-c4cccc(-c5ccc(F)cc5-c5nccn5C)c4)c(=O)c3[nH]2)C1 ; Cc1c[nH]nc1-c1cc(C#N)ccc1-c1cc(C2CC2)nc(-n2cc(C3CC3)c3cc(CN4C[C@@H]5C[C@H]4CN5)[nH]c3c2=O)c1 ; Cc1c[nH]nc1-c1cc(C#N)ccc1-c1cc(C2CC2)nc(-n2cc(C3CC3)c3cc(CN4CCC[C@H](C)C4)[nH]c3c2=O)c1 ; Cc1c[nH]nc1-c1cc(C#N)ccc1-c1cc(C2CC2)nc(-n2cc(C3CC3)c3cc(CNCC4(O)CCC4)[nH]c3c2=O)c1]

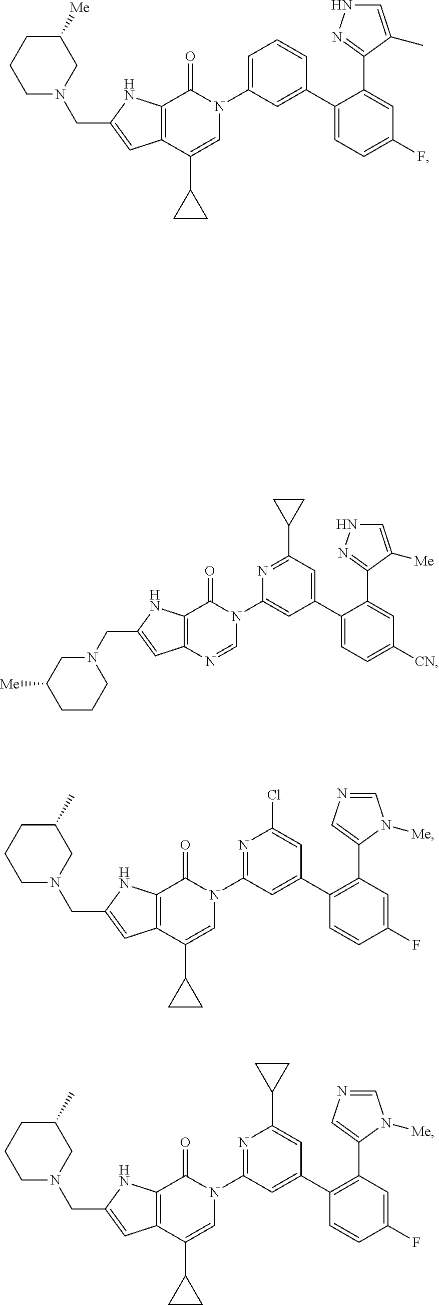 [SMILES: Cc1c[nH]nc1-c1cc(F)ccc1-c1cccc(-n2cc(C3CC3)c3cc(CN4CCC[C@H](C)C4)[nH]c3c2=O)c1 ; Cc1c[nH]nc1-c1cc(C#N)ccc1-c1cc(C2CC2)nc(-n2cnc3cc(CN4CCC[C@H](C)C4)[nH]c3c2=O)c1 ; C[C@H]1CCCN(Cc2cc3c(C4CC4)cn(-c4cc(-c5ccc(F)cc5-c5cncn5C)cc(Cl)n4)c(=O)c3[nH]2)C1 ; C[C@H]1CCCN(Cc2cc3c(C4CC4)cn(-c4cc(-c5ccc(F)cc5-c5cncn5C)cc(C5CC5)n4)c(=O)c3[nH]2)C1]

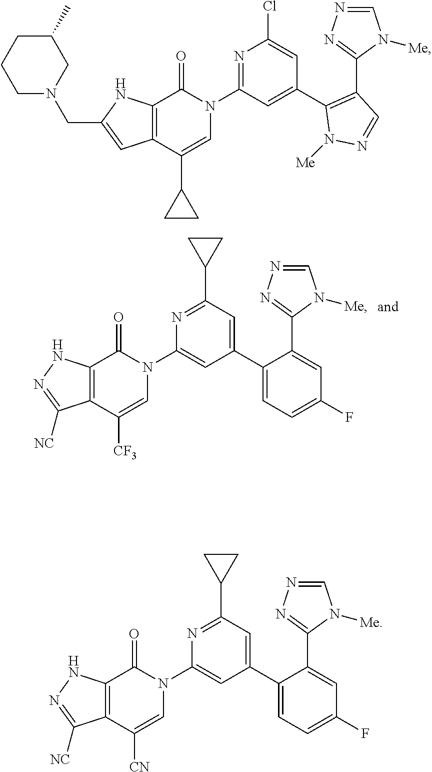

39. A compound selected from the group consisting of:

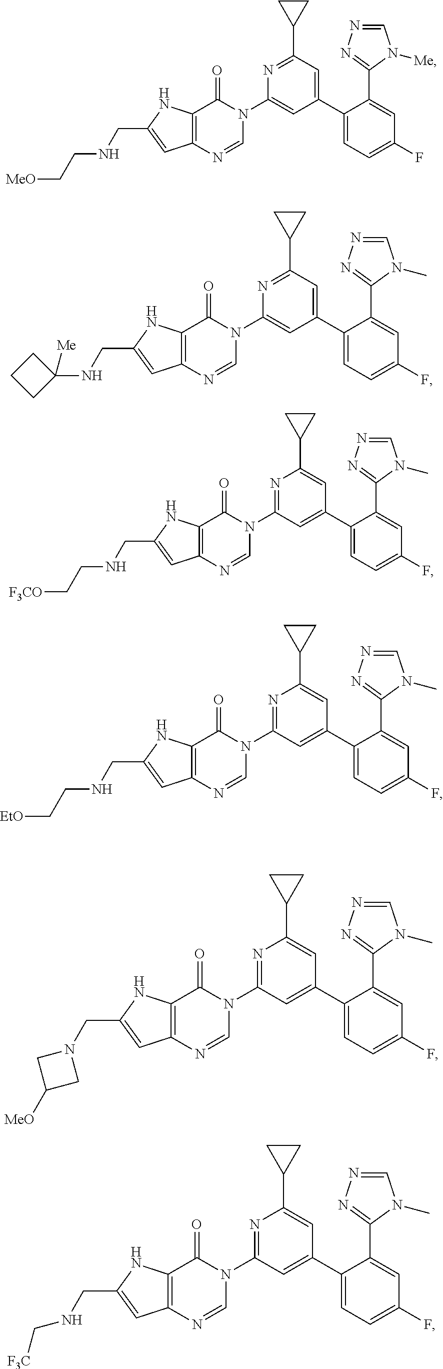 [SMILES: COCCNCc1cc2ncn(-c3cc(-c4ccc(F)cc4-c4nncn4C)cc(C4CC4)n3)c(=O)c2[nH]1 ; Cn1cnnc1-c1cc(F)ccc1-c1cc(C2CC2)nc(-n2cnc3cc(CNC4(C)CCC4)[nH]c3c2=O)c1 ; Cn1cnnc1-c1cc(F)ccc1-c1cc(C2CC2)nc(-n2cnc3cc(CNCCOC(F)(F)F)[nH]c3c2=O)c1 ; CCOCCNCc1cc2ncn(-c3cc(-c4ccc(F)cc4-c4nncn4C)cc(C4CC4)n3)c(=O)c2[nH]1 ; COC1CN(Cc2cc3ncn(-c4cc(-c5ccc(F)cc5-c5nncn5C)cc(C5CC5)n4)c(=O)c3[nH]2)C1 ; Cn1cnnc1-c1cc(F)ccc1-c1cc(C2CC2)nc(-n2cnc3cc(CNCC(F)(F)F)[nH]c3c2=O)c1]

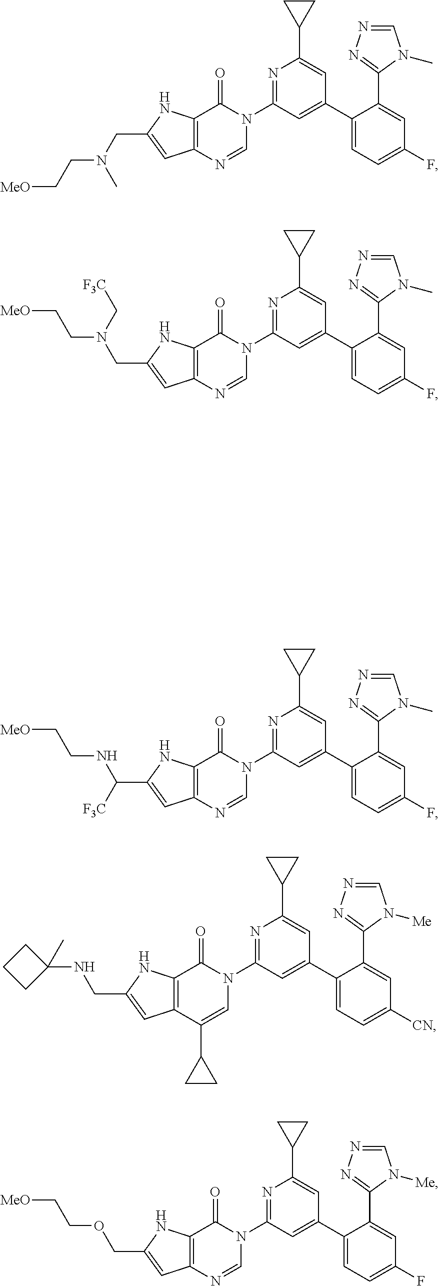 [SMILES: COCCN(C)Cc1cc2ncn(-c3cc(-c4ccc(F)cc4-c4nncn4C)cc(C4CC4)n3)c(=O)c2[nH]1 ; COCCN(Cc1cc2ncn(-c3cc(-c4ccc(F)cc4-c4nncn4C)cc(C4CC4)n3)c(=O)c2[nH]1)CC(F)(F)F ; COCCNC(c1cc2ncn(-c3cc(-c4ccc(F)cc4-c4nncn4C)cc(C4CC4)n3)c(=O)c2[nH]1)C(F)(F)F ; Cn1cnnc1-c1cc(C#N)ccc1-c1cc(C2CC2)nc(-n2cc(C3CC3)c3cc(CNC4(C)CCC4)[nH]c3c2=O)c1 ; COCCOCc1cc2ncn(-c3cc(-c4ccc(F)cc4-c4nncn4C)cc(C4CC4)n3)c(=O)c2[nH]1]

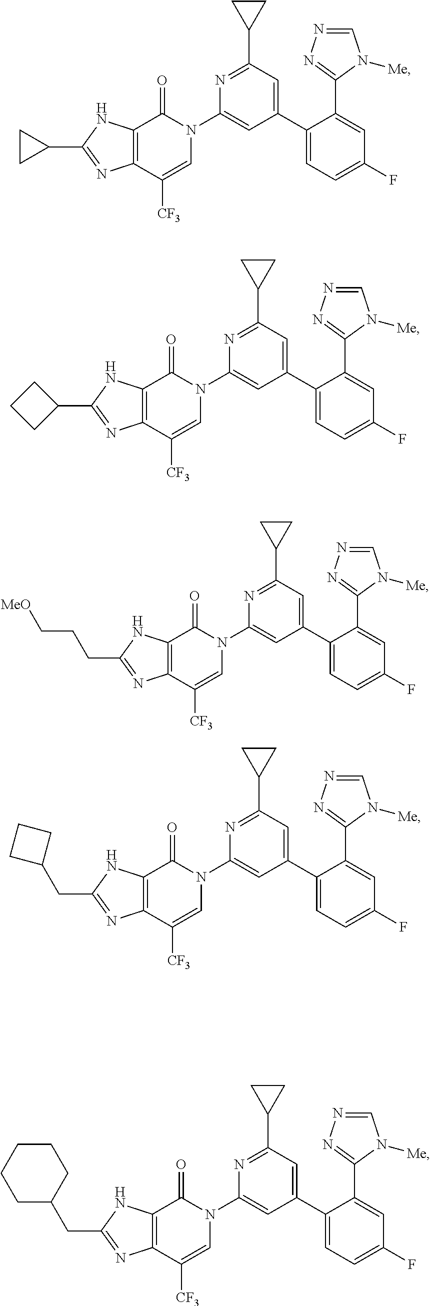 [SMILES: Cn1cnnc1-c1cc(F)ccc1-c1cc(C2CC2)nc(-n2cc(C(F)(F)F)c3nc(C4CC4)[nH]c3c2=O)c1 ; Cn1cnnc1-c1cc(F)ccc1-c1cc(C2CC2)nc(-n2cc(C(F)(F)F)c3nc(C4CCC4)[nH]c3c2=O)c1 ; COCCCc1nc2c(C(F)(F)F)cn(-c3cc(-c4ccc(F)cc4-c4nncn4C)cc(C4CC4)n3)c(=O)c2[nH]1 ; Cn1cnnc1-c1cc(F)ccc1-c1cc(C2CC2)nc(-n2cc(C(F)(F)F)c3nc(CC4CCC4)[nH]c3c2=O)c1 ; Cn1cnnc1-c1cc(F)ccc1-c1cc(C2CC2)nc(-n2cc(C(F)(F)F)c3nc(CC4CCCCC4)[nH]c3c2=O)c1]

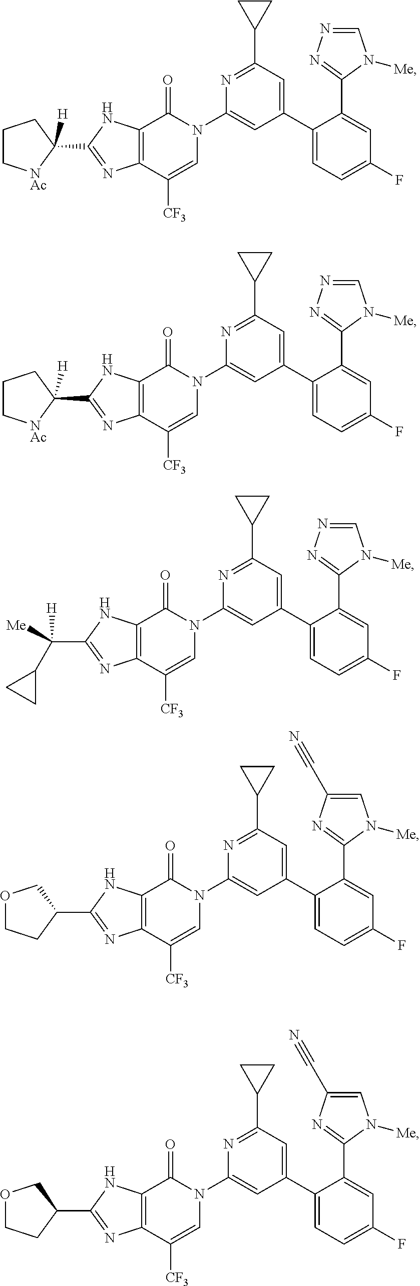

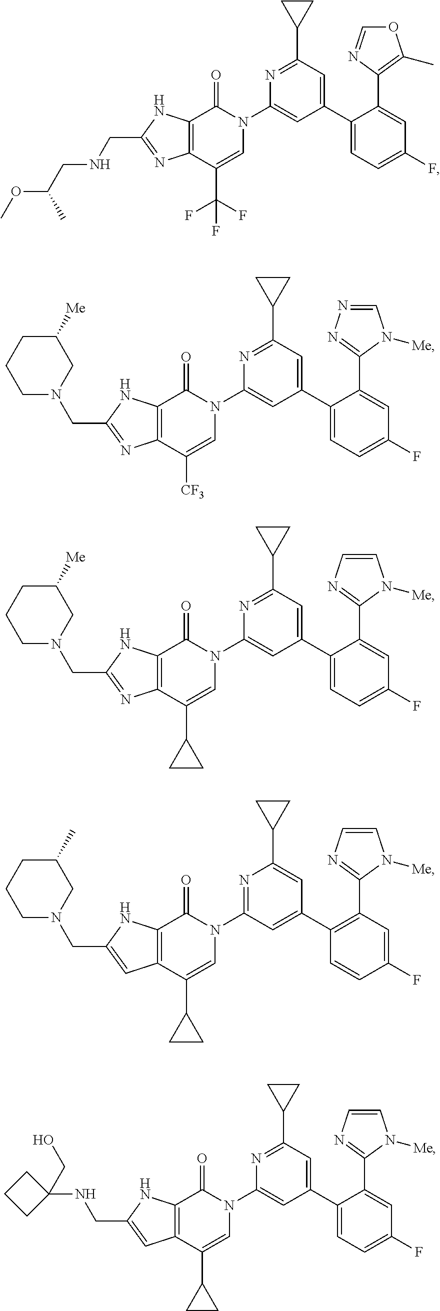

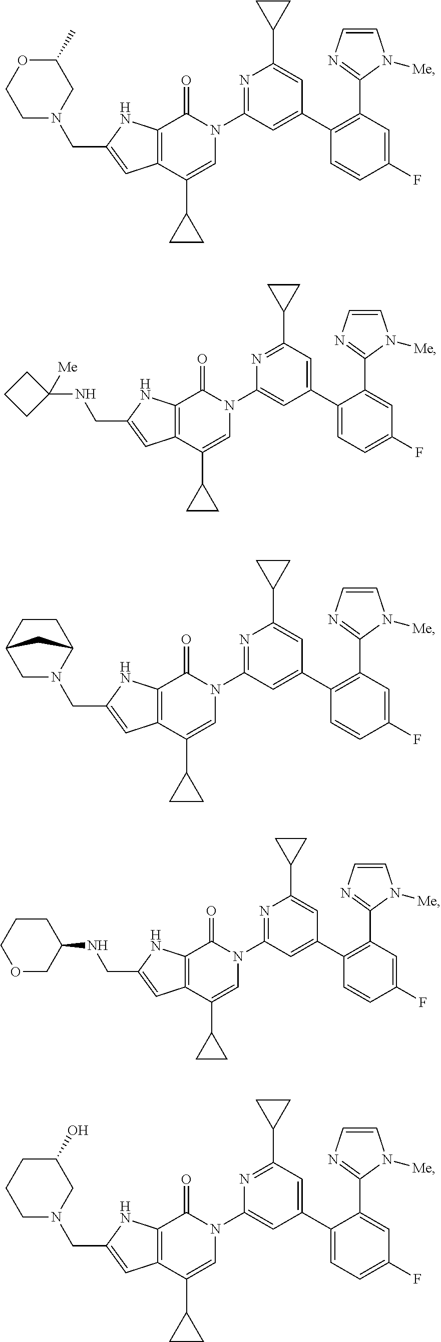 [SMILES: C[C@@H]1CN(Cc2cc3c(C4CC4)cn(-c4cc(-c5ccc(F)cc5-c5nccn5C)cc(C5CC5)n4)c(=O)c3[nH]2)CCO1 ; Cn1ccnc1-c1cc(F)ccc1-c1cc(C2CC2)nc(-n2cc(C3CC3)c3cc(CNC4(C)CCC4)[nH]c3c2=O)c1 ; Cn1ccnc1-c1cc(F)ccc1-c1cc(C2CC2)nc(-n2cc(C3CC3)c3cc(CN4C[C@@H]5CC[C@H]4C5)[nH]c3c2=O)c1 ; Cn1ccnc1-c1cc(F)ccc1-c1cc(C2CC2)nc(-n2cc(C3CC3)c3cc(CN[C@@H]4CCCOC4)[nH]c3c2=O)c1 ; Cn1ccnc1-c1cc(F)ccc1-c1cc(C2CC2)nc(-n2cc(C3CC3)c3cc(CN4CCC[C@H](O)C4)[nH]c3c2=O)c1]

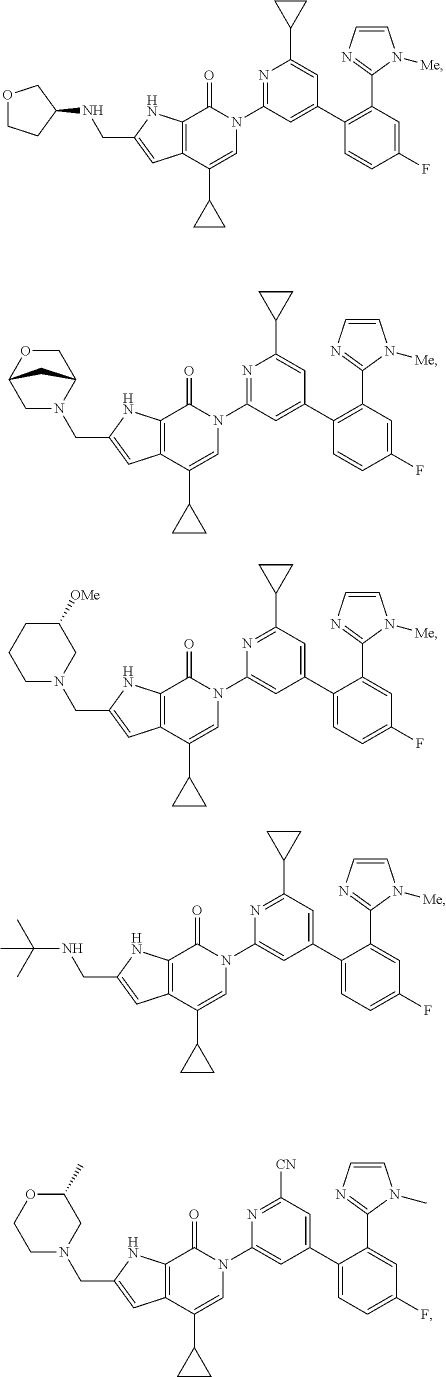 [SMILES: Cn1ccnc1-c1cc(F)ccc1-c1cc(C2CC2)nc(-n2cc(C3CC3)c3cc(CN[C@H]4CCOC4)[nH]c3c2=O)c1 ; Cn1ccnc1-c1cc(F)ccc1-c1cc(C2CC2)nc(-n2cc(C3CC3)c3cc(CN4C[C@H]5C[C@@H]4CO5)[nH]c3c2=O)c1 ; CO[C@H]1CCCN(Cc2cc3c(C4CC4)cn(-c4cc(-c5ccc(F)cc5-c5nccn5C)cc(C5CC5)n4)c(=O)c3[nH]2)C1 ; Cn1ccnc1-c1cc(F)ccc1-c1cc(C2CC2)nc(-n2cc(C3CC3)c3cc(CNC(C)(C)C)[nH]c3c2=O)c1 ; C[C@@H]1CN(Cc2cc3c(C4CC4)cn(-c4cc(-c5ccc(F)cc5-c5nccn5C)cc(C#N)n4)c(=O)c3[nH]2)CCO1]

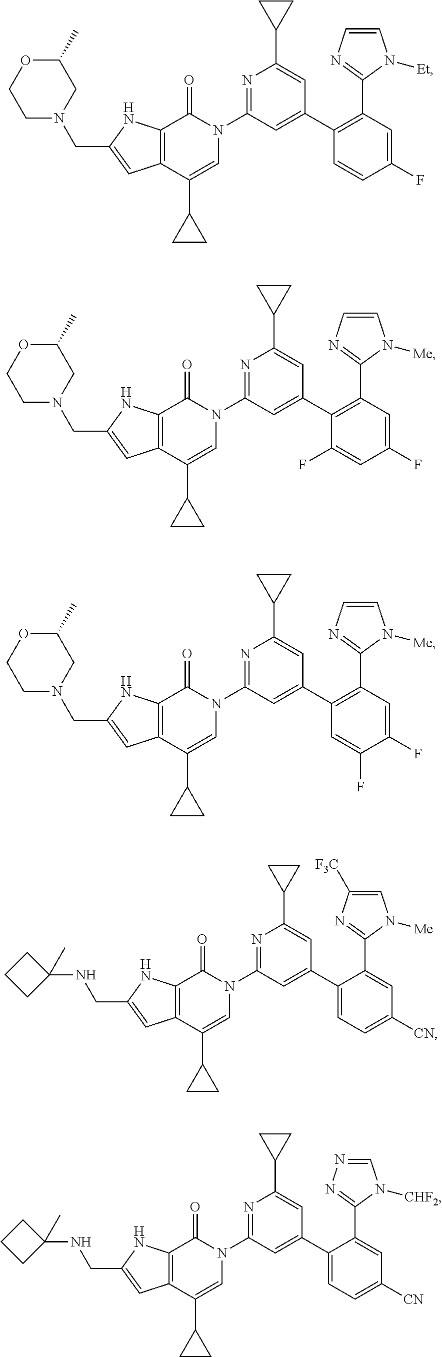 [SMILES: CCn1ccnc1-c1cc(F)ccc1-c1cc(C2CC2)nc(-n2cc(C3CC3)c3cc(CN4CCO[C@H](C)C4)[nH]c3c2=O)c1 ; C[C@@H]1CN(Cc2cc3c(C4CC4)cn(-c4cc(-c5c(F)cc(F)cc5-c5nccn5C)cc(C5CC5)n4)c(=O)c3[nH]2)CCO1 ; C[C@@H]1CN(Cc2cc3c(C4CC4)cn(-c4cc(-c5cc(F)c(F)cc5-c5nccn5C)cc(C5CC5)n4)c(=O)c3[nH]2)CCO1 ; Cn1cc(C(F)(F)F)nc1-c1cc(C#N)ccc1-c1cc(C2CC2)nc(-n2cc(C3CC3)c3cc(CNC4(C)CCC4)[nH]c3c2=O)c1 ; CC1(NCc2cc3c(C4CC4)cn(-c4cc(-c5ccc(C#N)cc5-c5nncn5C(F)F)cc(C5CC5)n4)c(=O)c3[nH]2)CCC1]

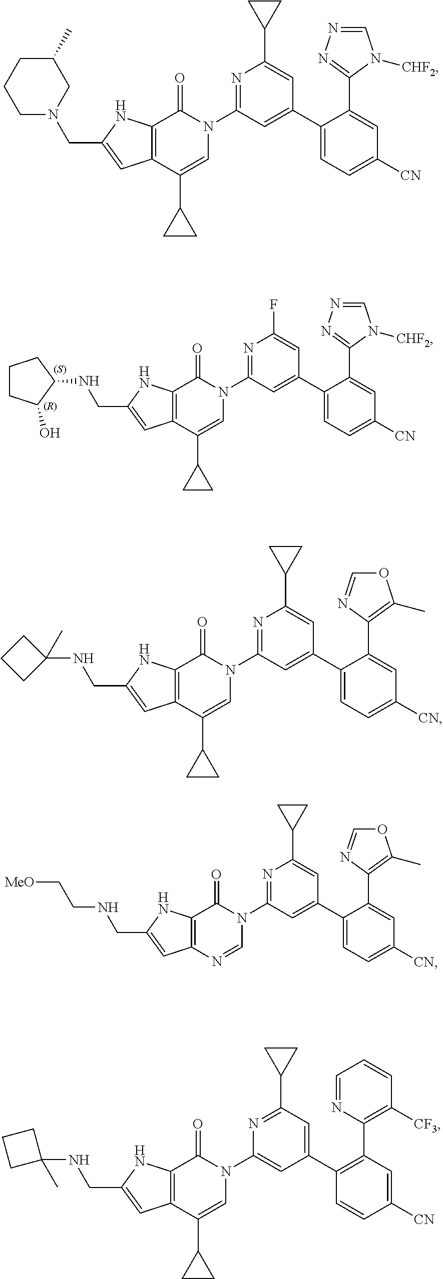

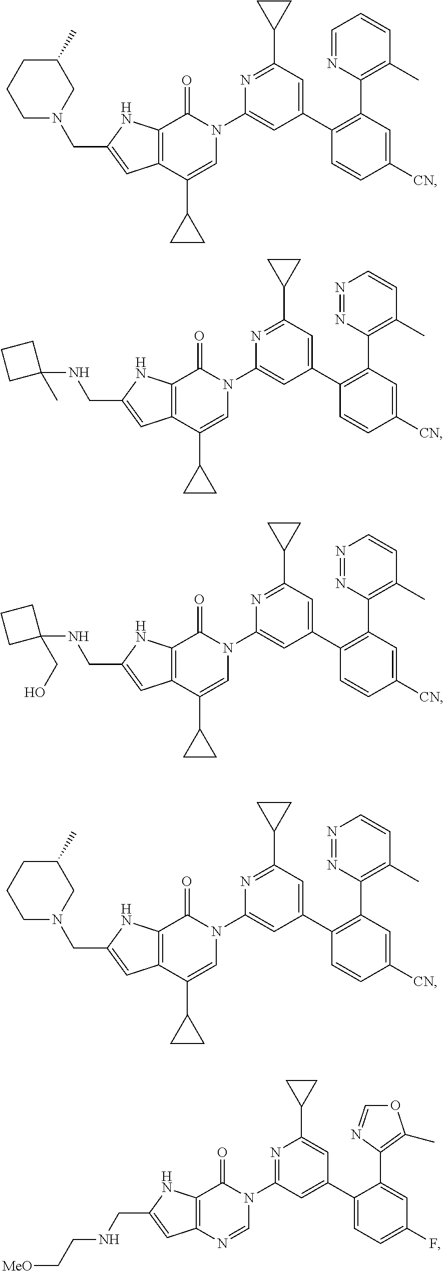 [SMILES: Cc1cccnc1-c1cc(C#N)ccc1-c1cc(C2CC2)nc(-n2cc(C3CC3)c3cc(CN4CCC[C@H](C)C4)[nH]c3c2=O)c1 ; Cc1ccnnc1-c1cc(C#N)ccc1-c1cc(C2CC2)nc(-n2cc(C3CC3)c3cc(CNC4(C)CCC4)[nH]c3c2=O)c1 ; Cc1ccnnc1-c1cc(C#N)ccc1-c1cc(C2CC2)nc(-n2cc(C3CC3)c3cc(CNC4(CO)CCC4)[nH]c3c2=O)c1 ; Cc1ccnnc1-c1cc(C#N)ccc1-c1cc(C2CC2)nc(-n2cc(C3CC3)c3cc(CN4CCC[C@H](C)C4)[nH]c3c2=O)c1 ; COCCNCc1cc2ncn(-c3cc(-c4ccc(F)cc4-c4ncoc4C)cc(C4CC4)n3)c(=O)c2[nH]1]

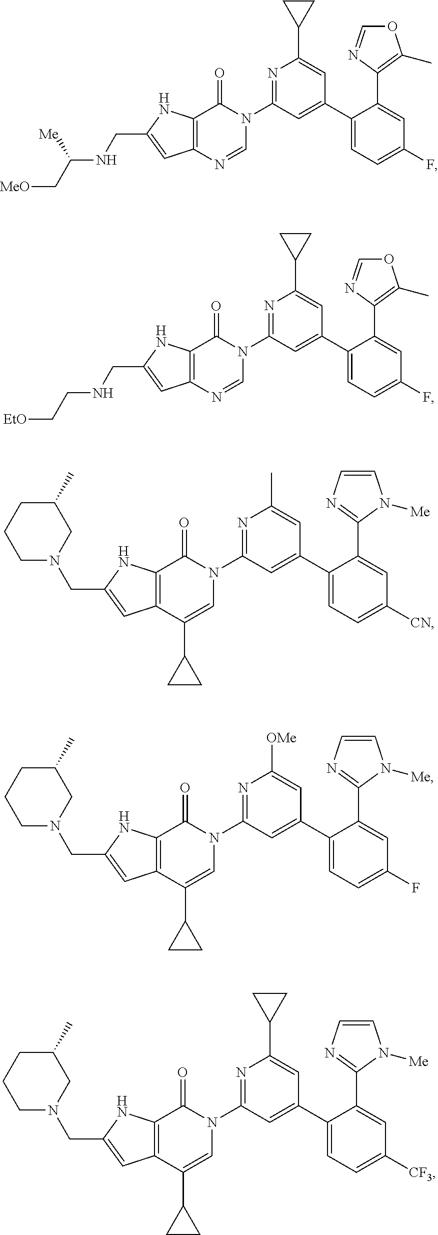 [SMILES: COC[C@H](C)NCc1cc2ncn(-c3cc(-c4ccc(F)cc4-c4ncoc4C)cc(C4CC4)n3)c(=O)c2[nH]1 ; CCOCCNCc1cc2ncn(-c3cc(-c4ccc(F)cc4-c4ncoc4C)cc(C4CC4)n3)c(=O)c2[nH]1 ; Cc1cc(-c2ccc(C#N)cc2-c2nccn2C)cc(-n2cc(C3CC3)c3cc(CN4CCC[C@H](C)C4)[nH]c3c2=O)n1 ; COc1cc(-c2ccc(F)cc2-c2nccn2C)cc(-n2cc(C3CC3)c3cc(CN4CCC[C@H](C)C4)[nH]c3c2=O)n1 ; C[C@H]1CCCN(Cc2cc3c(C4CC4)cn(-c4cc(-c5ccc(C(F)(F)F)cc5-c5nccn5C)cc(C5CC5)n4)c(=O)c3[nH]2)C1]

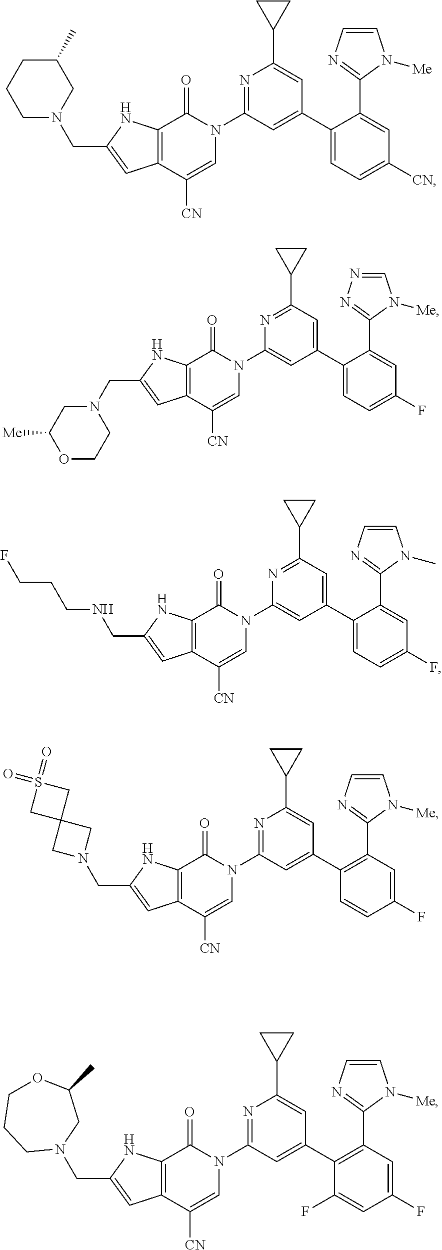 [SMILES: C[C@H]1CCCN(Cc2cc3c(C#N)cn(-c4cc(-c5ccc(C#N)cc5-c5nccn5C)cc(C5CC5)n4)c(=O)c3[nH]2)C1 ; C[C@@H]1CN(Cc2cc3c(C#N)cn(-c4cc(-c5ccc(F)cc5-c5nncn5C)cc(C5CC5)n4)c(=O)c3[nH]2)CCO1 ; Cn1ccnc1-c1cc(F)ccc1-c1cc(C2CC2)nc(-n2cc(C#N)c3cc(CNCCCF)[nH]c3c2=O)c1 ; Cn1ccnc1-c1cc(F)ccc1-c1cc(C2CC2)nc(-n2cc(C#N)c3cc(CN4CC5(C4)CS(=O)(=O)C5)[nH]c3c2=O)c1 ; C[C@H]1CN(Cc2cc3c(C#N)cn(-c4cc(-c5c(F)cc(F)cc5-c5nccn5C)cc(C5CC5)n4)c(=O)c3[nH]2)CCCO1]

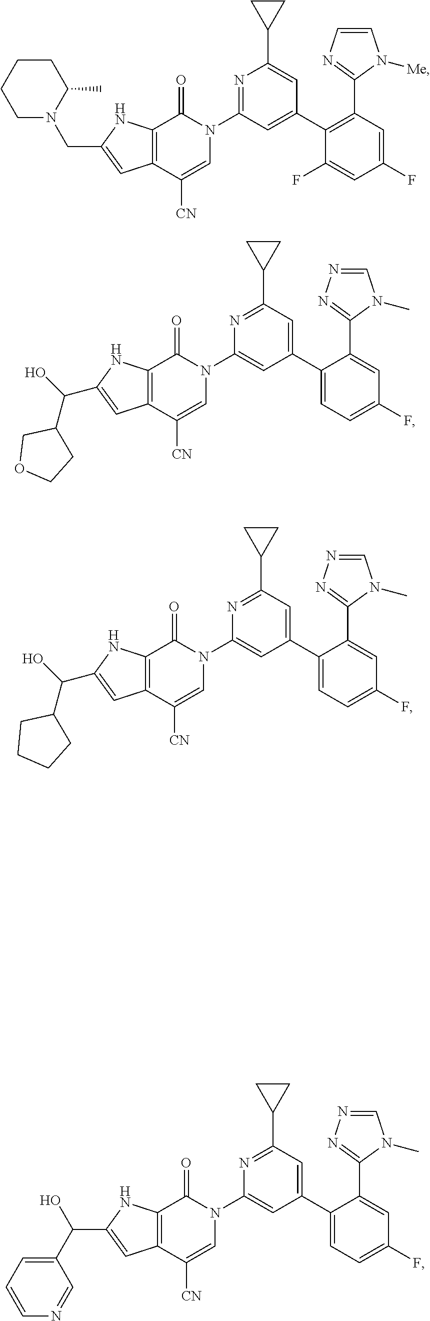 [SMILES: C[C@H]1CCCCN1Cc1cc2c(C#N)cn(-c3cc(-c4c(F)cc(F)cc4-c4nccn4C)cc(C4CC4)n3)c(=O)c2[nH]1 ; Cn1cnnc1-c1cc(F)ccc1-c1cc(C2CC2)nc(-n2cc(C#N)c3cc(C(O)C4CCOC4)[nH]c3c2=O)c1 ; Cn1cnnc1-c1cc(F)ccc1-c1cc(C2CC2)nc(-n2cc(C#N)c3cc(C(O)C4CCCC4)[nH]c3c2=O)c1 ; Cn1cnnc1-c1cc(F)ccc1-c1cc(C2CC2)nc(-n2cc(C#N)c3cc(C(O)c4cccnc4)[nH]c3c2=O)c1]

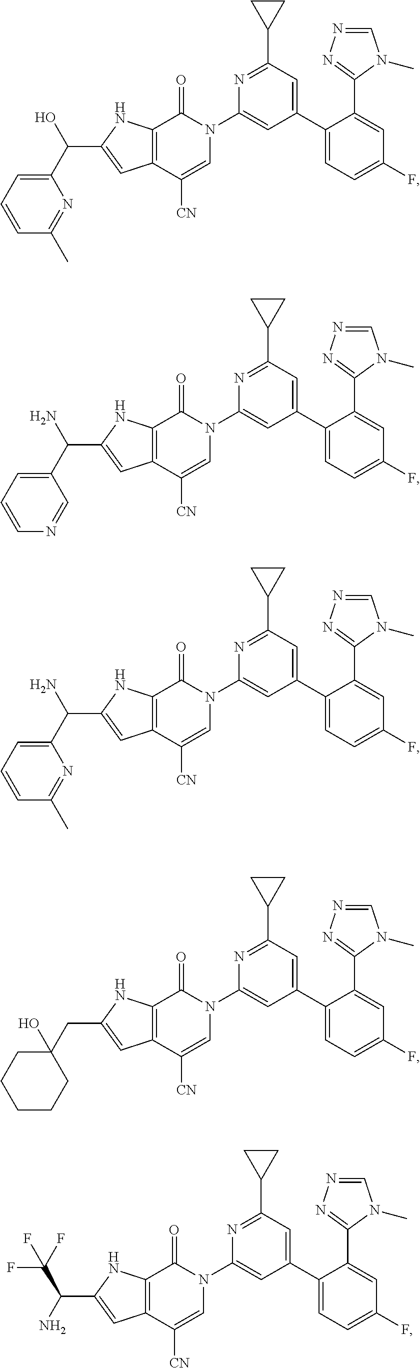 [SMILES: Cc1cccc(C(O)c2cc3c(C#N)cn(-c4cc(-c5ccc(F)cc5-c5nncn5C)cc(C5CC5)n4)c(=O)c3[nH]2)n1 ; Cn1cnnc1-c1cc(F)ccc1-c1cc(C2CC2)nc(-n2cc(C#N)c3cc(C(N)c4cccnc4)[nH]c3c2=O)c1 ; Cc1cccc(C(N)c2cc3c(C#N)cn(-c4cc(-c5ccc(F)cc5-c5nncn5C)cc(C5CC5)n4)c(=O)c3[nH]2)n1 ; Cn1cnnc1-c1cc(F)ccc1-c1cc(C2CC2)nc(-n2cc(C#N)c3cc(CC4(O)CCCCC4)[nH]c3c2=O)c1 ; Cn1cnnc1-c1cc(F)ccc1-c1cc(C2CC2)nc(-n2cc(C#N)c3cc([C@@H](N)C(F)(F)F)[nH]c3c2=O)c1]

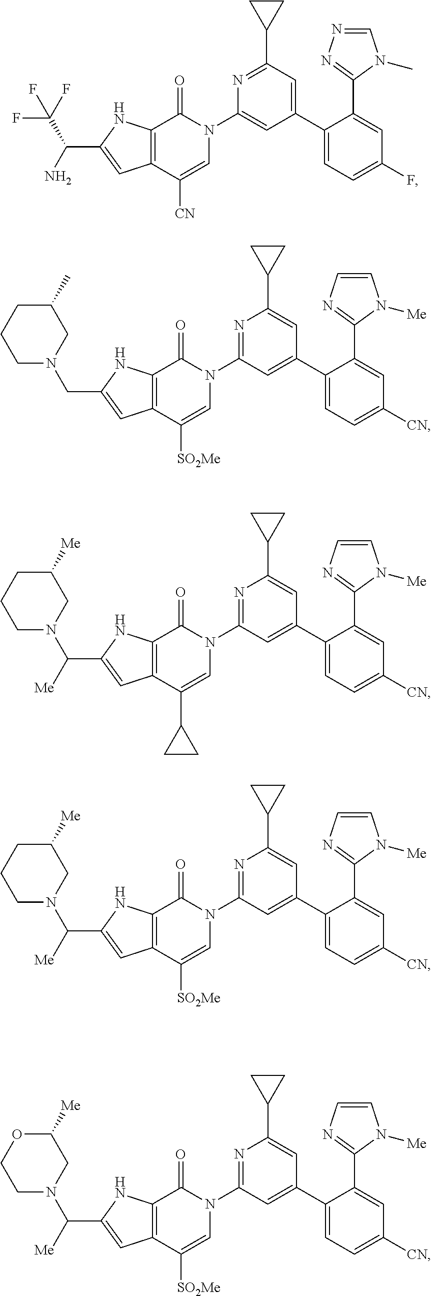 [SMILES: Cn1cnnc1-c1cc(F)ccc1-c1cc(C2CC2)nc(-n2cc(C#N)c3cc([C@H](N)C(F)(F)F)[nH]c3c2=O)c1 ; C[C@H]1CCCN(Cc2cc3c(S(C)(=O)=O)cn(-c4cc(-c5ccc(C#N)cc5-c5nccn5C)cc(C5CC5)n4)c(=O)c3[nH]2)C1 ; CC(c1cc2c(C3CC3)cn(-c3cc(-c4ccc(C#N)cc4-c4nccn4C)cc(C4CC4)n3)c(=O)c2[nH]1)N1CCC[C@H](C)C1 ; CC(c1cc2c(S(C)(=O)=O)cn(-c3cc(-c4ccc(C#N)cc4-c4nccn4C)cc(C4CC4)n3)c(=O)c2[nH]1)N1CCC[C@H](C)C1 ; CC(c1cc2c(S(C)(=O)=O)cn(-c3cc(-c4ccc(C#N)cc4-c4nccn4C)cc(C4CC4)n3)c(=O)c2[nH]1)N1CCO[C@H](C)C1]

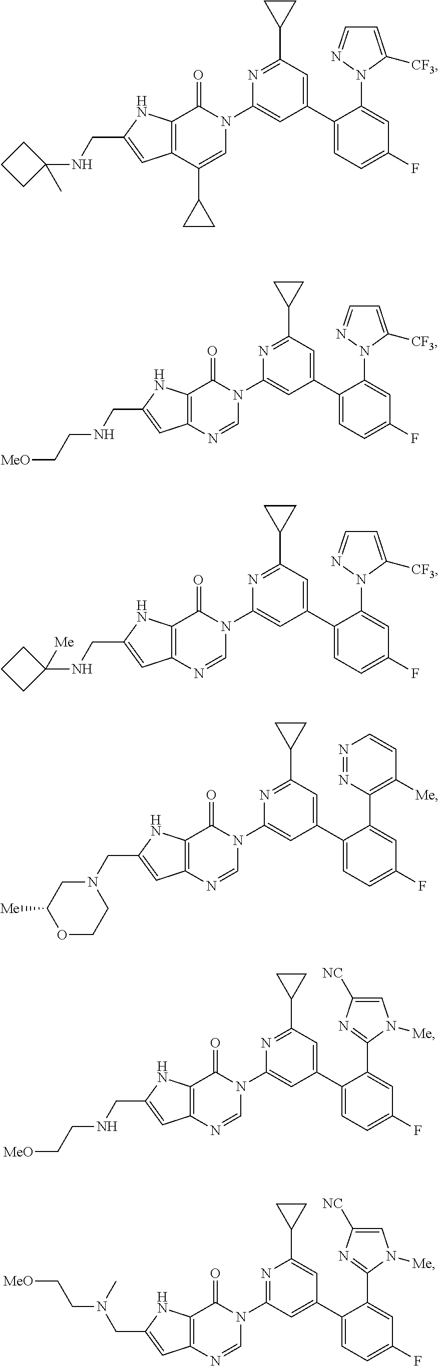 [SMILES: CC1(NCc2cc3c(C4CC4)cn(-c4cc(-c5ccc(F)cc5-n5nccc5C(F)(F)F)cc(C5CC5)n4)c(=O)c3[nH]2)CCC1 ; COCCNCc1cc2ncn(-c3cc(-c4ccc(F)cc4-n4nccc4C(F)(F)F)cc(C4CC4)n3)c(=O)c2[nH]1 ; CC1(NCc2cc3ncn(-c4cc(-c5ccc(F)cc5-n5nccc5C(F)(F)F)cc(C5CC5)n4)c(=O)c3[nH]2)CCC1 ; Cc1ccnnc1-c1cc(F)ccc1-c1cc(C2CC2)nc(-n2cnc3cc(CN4CCO[C@H](C)C4)[nH]c3c2=O)c1 ; COCCNCc1cc2ncn(-c3cc(-c4ccc(F)cc4-c4nc(C#N)cn4C)cc(C4CC4)n3)c(=O)c2[nH]1 ; COCCN(C)Cc1cc2ncn(-c3cc(-c4ccc(F)cc4-c4nc(C#N)cn4C)cc(C4CC4)n3)c(=O)c2[nH]1]

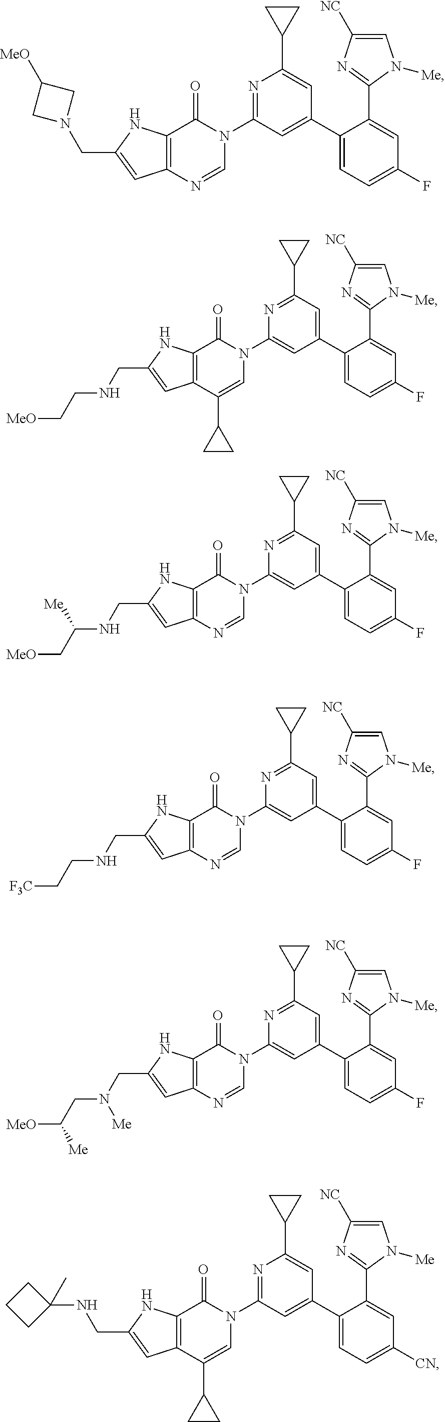 [SMILES: COC1CN(Cc2cc3ncn(-c4cc(-c5ccc(F)cc5-c5nc(C#N)cn5C)cc(C5CC5)n4)c(=O)c3[nH]2)C1 ; COCCNCc1cc2c(C3CC3)cn(-c3cc(-c4ccc(F)cc4-c4nc(C#N)cn4C)cc(C4CC4)n3)c(=O)c2[nH]1 ; COC[C@H](C)NCc1cc2ncn(-c3cc(-c4ccc(F)cc4-c4nc(C#N)cn4C)cc(C4CC4)n3)c(=O)c2[nH]1 ; Cn1cc(C#N)nc1-c1cc(F)ccc1-c1cc(C2CC2)nc(-n2cnc3cc(CNCCC(F)(F)F)[nH]c3c2=O)c1 ; CO[C@@H](C)CN(C)Cc1cc2ncn(-c3cc(-c4ccc(F)cc4-c4nc(C#N)cn4C)cc(C4CC4)n3)c(=O)c2[nH]1 ; Cn1cc(C#N)nc1-c1cc(C#N)ccc1-c1cc(C2CC2)nc(-n2cc(C3CC3)c3cc(CNC4(C)CCC4)[nH]c3c2=O)c1]

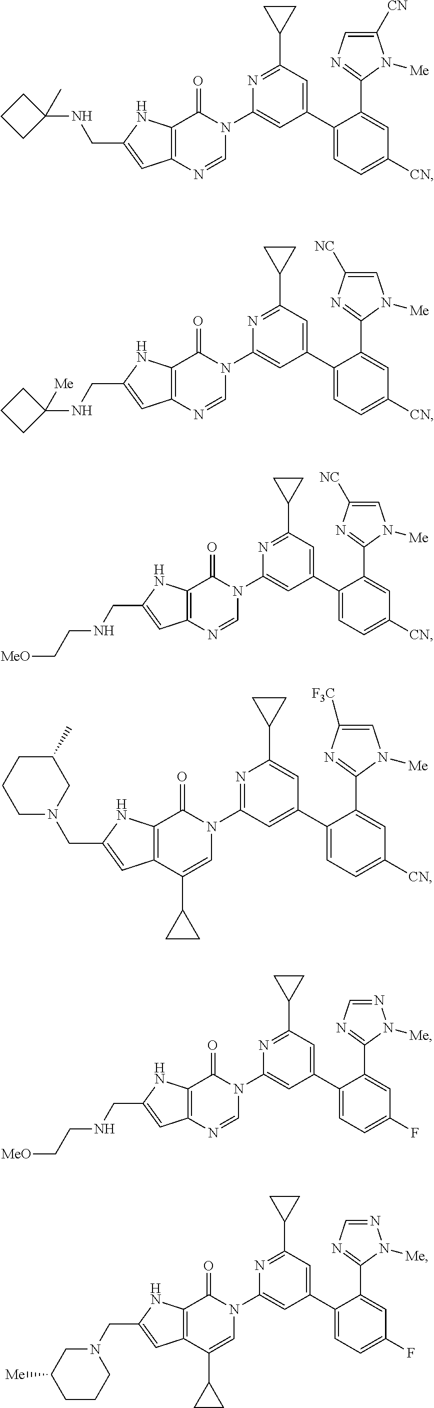 [SMILES: Cn1c(C#N)cnc1-c1cc(C#N)ccc1-c1cc(C2CC2)nc(-n2cnc3cc(CNC4(C)CCC4)[nH]c3c2=O)c1 ; Cn1cc(C#N)nc1-c1cc(C#N)ccc1-c1cc(C2CC2)nc(-n2cnc3cc(CNC4(C)CCC4)[nH]c3c2=O)c1 ; COCCNCc1cc2ncn(-c3cc(-c4ccc(C#N)cc4-c4nc(C#N)cn4C)cc(C4CC4)n3)c(=O)c2[nH]1 ; C[C@H]1CCCN(Cc2cc3c(C4CC4)cn(-c4cc(-c5ccc(C#N)cc5-c5nc(C(F)(F)F)cn5C)cc(C5CC5)n4)c(=O)c3[nH]2)C1 ; COCCNCc1cc2ncn(-c3cc(-c4ccc(F)cc4-c4ncnn4C)cc(C4CC4)n3)c(=O)c2[nH]1 ; C[C@H]1CCCN(Cc2cc3c(C4CC4)cn(-c4cc(-c5ccc(F)cc5-c5ncnn5C)cc(C5CC5)n4)c(=O)c3[nH]2)C1]

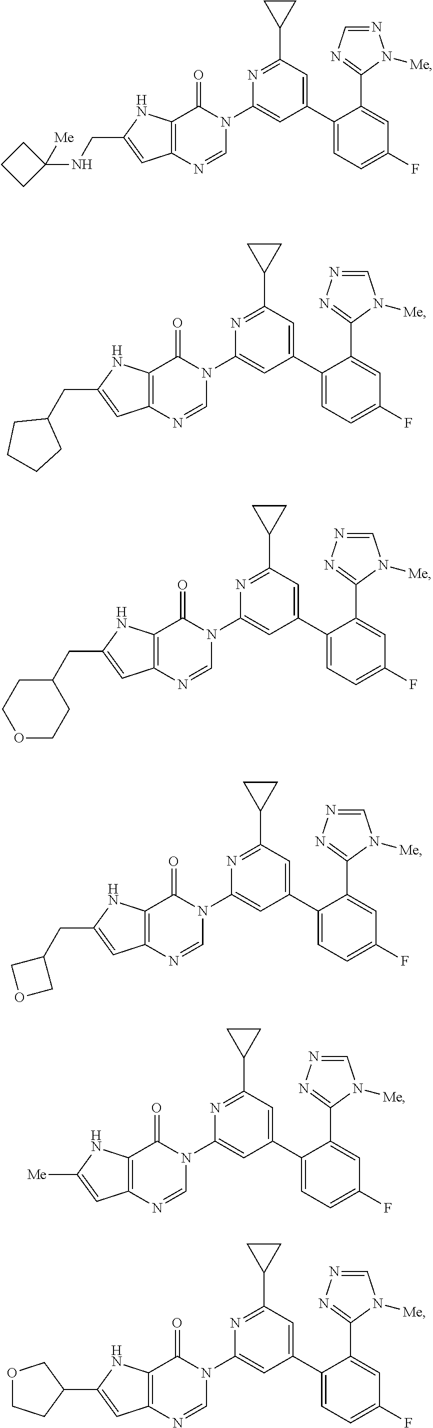 [SMILES: Cn1ncnc1-c1cc(F)ccc1-c1cc(C2CC2)nc(-n2cnc3cc(CNC4(C)CCC4)[nH]c3c2=O)c1 ; Cn1cnnc1-c1cc(F)ccc1-c1cc(C2CC2)nc(-n2cnc3cc(CC4CCCC4)[nH]c3c2=O)c1 ; Cn1cnnc1-c1cc(F)ccc1-c1cc(C2CC2)nc(-n2cnc3cc(CC4CCOCC4)[nH]c3c2=O)c1 ; Cn1cnnc1-c1cc(F)ccc1-c1cc(C2CC2)nc(-n2cnc3cc(CC4COC4)[nH]c3c2=O)c1 ; Cc1cc2ncn(-c3cc(-c4ccc(F)cc4-c4nncn4C)cc(C4CC4)n3)c(=O)c2[nH]1 ; Cn1cnnc1-c1cc(F)ccc1-c1cc(C2CC2)nc(-n2cnc3cc(C4CCOC4)[nH]c3c2=O)c1]

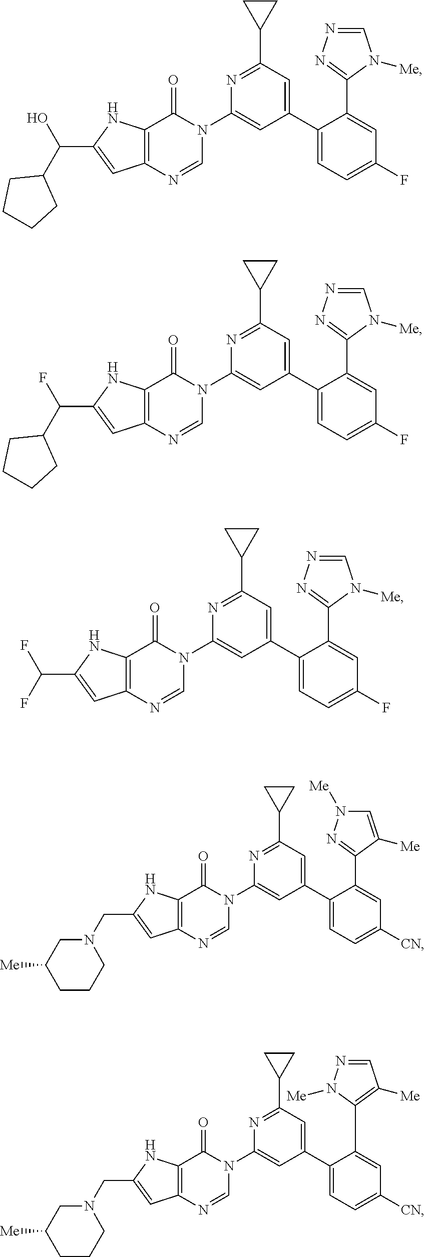 [SMILES: Cn1cnnc1-c1cc(F)ccc1-c1cc(C2CC2)nc(-n2cnc3cc(C(O)C4CCCC4)[nH]c3c2=O)c1 ; Cn1cnnc1-c1cc(F)ccc1-c1cc(C2CC2)nc(-n2cnc3cc(C(F)C4CCCC4)[nH]c3c2=O)c1 ; Cn1cnnc1-c1cc(F)ccc1-c1cc(C2CC2)nc(-n2cnc3cc(C(F)F)[nH]c3c2=O)c1 ; Cc1cn(C)nc1-c1cc(C#N)ccc1-c1cc(C2CC2)nc(-n2cnc3cc(CN4CCC[C@H](C)C4)[nH]c3c2=O)c1 ; Cc1cnn(C)c1-c1cc(C#N)ccc1-c1cc(C2CC2)nc(-n2cnc3cc(CN4CCC[C@H](C)C4)[nH]c3c2=O)c1]

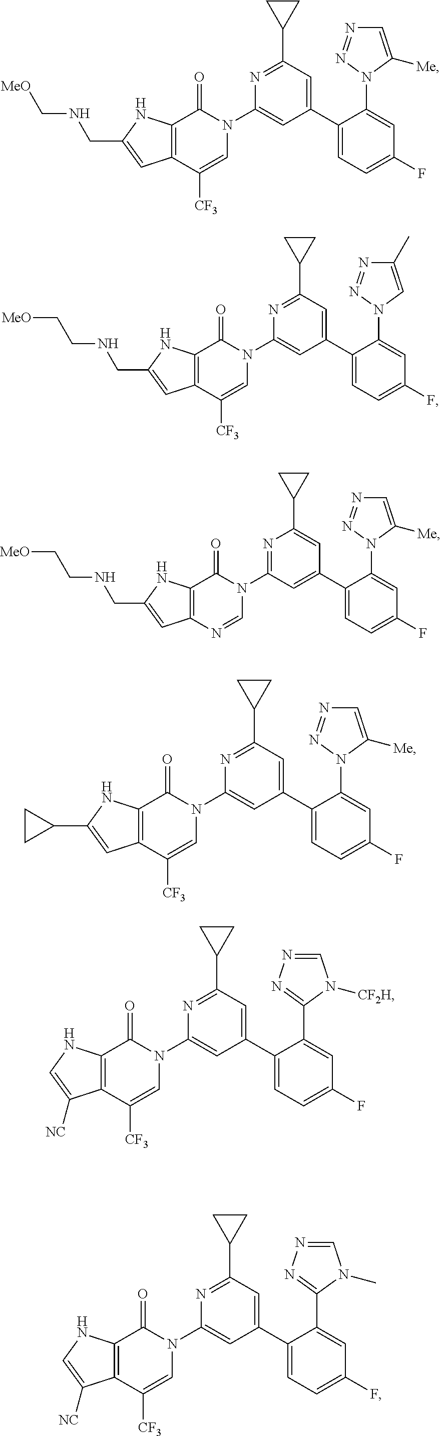 [SMILES: COCNCc1cc2c(C(F)(F)F)cn(-c3cc(-c4ccc(F)cc4-n4nncc4C)cc(C4CC4)n3)c(=O)c2[nH]1 ; COCCNCc1cc2c(C(F)(F)F)cn(-c3cc(-c4ccc(F)cc4-n4cc(C)nn4)cc(C4CC4)n3)c(=O)c2[nH]1 ; COCCNCc1cc2ncn(-c3cc(-c4ccc(F)cc4-n4nncc4C)cc(C4CC4)n3)c(=O)c2[nH]1 ; Cc1cnnn1-c1cc(F)ccc1-c1cc(C2CC2)nc(-n2cc(C(F)(F)F)c3cc(C4CC4)[nH]c3c2=O)c1 ; N#Cc1c[nH]c2c(=O)n(-c3cc(-c4ccc(F)cc4-c4nncn4C(F)F)cc(C4CC4)n3)cc(C(F)(F)F)c12 ; Cn1cnnc1-c1cc(F)ccc1-c1cc(C2CC2)nc(-n2cc(C(F)(F)F)c3c(C#N)c[nH]c3c2=O)c1]

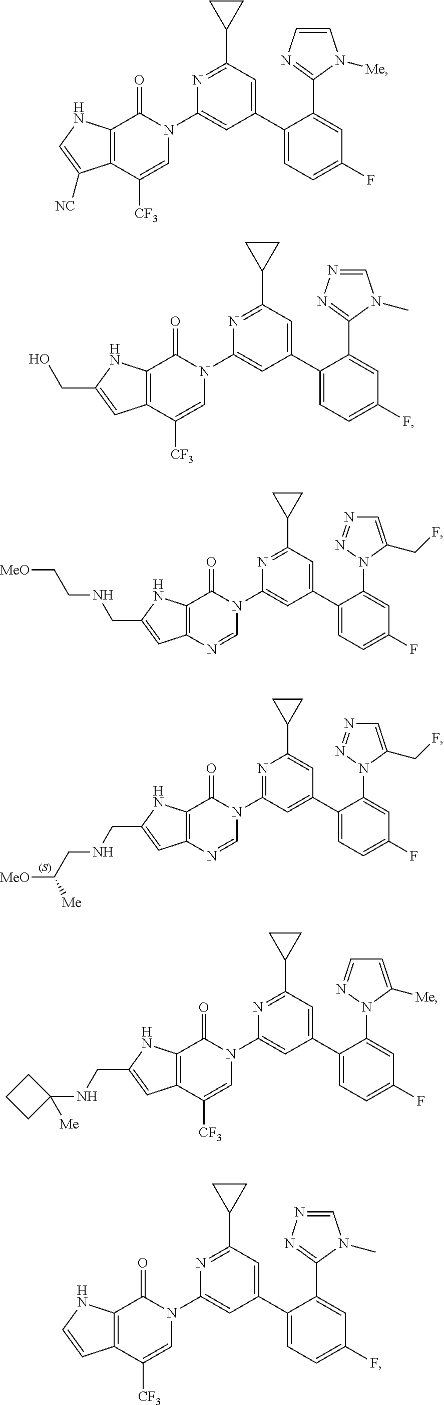

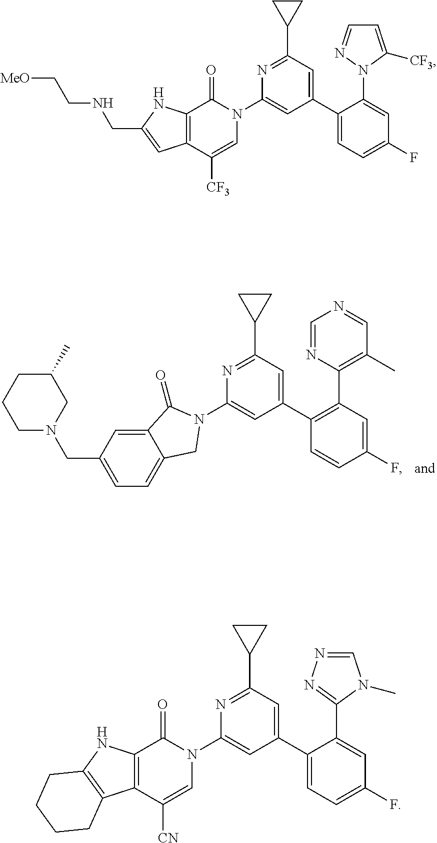 [SMILES: COCCNCc1cc2c(C(F)(F)F)cn(-c3cc(-c4ccc(F)cc4-n4nccc4C(F)(F)F)cc(C4CC4)n3)c(=O)c2[nH]1 ; Cc1cncnc1-c1cc(F)ccc1-c1cc(C2CC2)nc(N2Cc3ccc(CN4CCC[C@H](C)C4)cc3C2=O)c1 ; "and" ; Cn1cnnc1-c1cc(F)ccc1-c1cc(C2CC2)nc(-n2cc(C#N)c3c4c([nH]c3c2=O)CCCC4)c1]

40. A pharmaceutical composition comprising a compound of claim 1, or a pharmaceutically acceptable salt thereof, and a pharmaceutically acceptable excipient.

41. (canceled)

42. (canceled)

43. A method of treating a disease, disorder, or condition mediated at least in part by Cbl-b in a subject in need thereof, the method comprising administering to the subject a therapeutically effective amount of a compound of claim 1, or the pharmaceutically acceptable salt thereof.

44. (canceled)

45. The method of claim 43, wherein the disease, disorder, or condition is cancer.

46. The method of claim 45, wherein the cancer is:
a) a cancer of the genitourinary tract (e.g., gynecologic, bladder, kidney, renal cell, penile, prostate, or testicular), breast, gastrointestinal tract (e.g., esophagus, oropharynx, stomach, small or large intestines, colon, or rectum), bone, bone marrow, skin (e.g., melanoma), head and neck, liver, gall bladder, bile ducts, heart, lung, pancreas, salivary gland, adrenal gland, thyroid, brain (e.g., gliomas), ganglia, central nervous system (CNS), peripheral nervous system (PNS), the hematopoietic system (i.e., hematological malignancies), or the immune system (e.g., spleen or thymus), or any combination thereof;
b) breast cancer, genitourinary cancer, gastrointestinal cancer, lung cancer, skin cancer, or a combination thereof; or
c) a hematological malignancy, optionally selected from leukemias, lymphomas and myelomas.

47. (canceled)

48. (canceled)

49. The method of claim 45, further comprising administering at least one additional therapeutic agent to the subject.

50. The method of claim 49, wherein said at least one additional therapeutic agent comprises one or more agents independently selected from the groups consisting of immune checkpoint inhibitors, agents that target the extracellular production of adenosine, inhibitors of HIF (e.g., a HIF-2α inhibitor), tyrosine kinase inhibitors, radiation therapy, and chemotherapeutic agents.

51-84. (canceled)
